SEQUENCE LISTING (4 sequences)

SEQ ID NO: 1 - DNA, 23 bp, synthetic construct
  source 1..23: mol_type = other DNA
tgccttccccactaccctaacaa

SEQ ID NO: 2 - DNA, 24 bp, synthetic construct
  source 1..24: mol_type = other DNA
tacactcccctcccactccactat

SEQ ID NO: 3 - DNA, 22 bp, synthetic construct
  source 1..22: mol_type = other DNA
gggctccttgggtaatgtatcc

SEQ ID NO: 4 - DNA, 24 bp, synthetic construct
  source 1..24: mol_type = other DNA
attctcagggcggtaggttagtcc

(19) United States
(12) Patent Application Publication
(10) Pub. No.: US 2026/0001886 A1
(43) Pub. Date: Jan. 1, 2026

(54) ENPP1 INHIBITORS I

(71) Applicant: RE Ventures I, LLC, Wilmington, DE (US)

(72) Inventors: Andrew Simon Bell, Deal (GB); Simone Culurgioni, Oxford (GB); Olivier Rémi Barbeau, Didcot (GB); Giulia Chemi, Reading (GB); Victor Sebastián-Pérez, Oxford (GB)

(21) Appl. No.: 19/201,240

(22) Filed: May 7, 2025

(30) Foreign Application Priority Data
May 7, 2024 (GB) 2406316.6
Sep. 16, 2024 (GB) 2413623.6

(51) Int. Cl. C07D 487/04; A61K 31/519; A61P 3/00; A61P 19/08; C07D 491/147; C07D 519/00

(52) U.S. Cl. CPC C07D 487/04; A61K 31/519; A61P 3/00; A61P 19/08; C07D 491/147; C07D 519/00

(57) ABSTRACT

Disclosed are compounds, compositions and methods for treating disease, syndromes, conditions and disorders that are affected by the inhibition of ENPP1. Such compounds are represented by Formula (I), wherein the variables are defined herein.

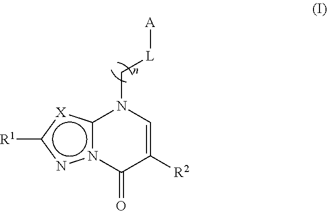

### TECHNICAL FIELD

The present disclosure relates to novel compounds capable of modulating ENPP1 hydrolytic activity. Such hydrolytic activity may be inhibited by the compounds described herein. The present invention further describes the synthesis of the compounds and their uses as medicaments in diseases or disorders where ENPP1 inhibition may be beneficial.

### INCORPORATION BY REFERENCE OF THE SEQUENCE LISTING

This application contains, as a separate part of disclosure, a Sequence Listing in computer-readable form (Filename: P75566US-ST.26 SEQL.xml; Size: 4,336 bytes; Created: Apr. 8, 2025) which is incorporated by reference herein in its entirety.

### BACKGROUND

Ectonucleotide Pyrophosphatase/Phosphodiesterase 1 (ENPP1), also known as ARHR2, COLED, M6S1, NPP1, NPPS, PC-1, PCA1 and PDNP1, is an enzyme that hydrolyses phosphorylated nucleotides, including adenosine triphosphate (ATP) and 2′,3′-cyclic adenosine monophosphate-guanidine monophosphate (cGAMP), while generating inorganic pyrophosphates (PPi) (Roberts F, Zhu D, Farquharson C, Macrae V E; Trends Biochem Sci. 2019 July; 44 (7): 616-628). Inorganic pyrophosphates, amongst other things, inhibit bone and cartilage mineralization. Therefore, the generation of PPi by ENPP1 inadvertently makes ENPP1 a central regulator of bone and cartilage development in mammals and many inherited mineralisation, calcium handling or calcification-related disorders have been linked to gain or loss-of-function mutations in ENPP1. For example, hypophosphatasia (HPP), is a rare genetic disorder that is characterized by impaired mineralization (“calcification”) of bones and teeth. Significant and numerous health problems can occur because mineralization is the process by which bones and teeth take up calcium and phosphorus required for proper hardness and strength. Skeletal and dental hypomineralization characterizes HPP, with disease severity varying from the life-threatening perinatal and infantile forms to milder (non-lethal) later-onset forms that manifest in adulthood or only affect the dentition. The incidence of severe forms of HPP ranges from 1 in 100,000 (US and Canada) to 1 in 300,000 (EU), whilst the incidence of less severe forms ranges from 1 in 2,430 to 1 in 508.

HPP is caused by loss-of-function mutations in the gene (ALPL in humans; Alpl in mice) that encodes tissue-nonspecific alkaline phosphatase (TNAP or TNSALP), whose deficiency results in the accumulation of extracellular PPi.

In particular, small molecule inhibitors of ENPP1 may be able to reduce levels of inorganic pyrophosphate (PPi) in the human or animal body through the controlled inhibition of ENPP1, and may in turn be able to restore the balance of PPi and phosphate so as to promote proper bone mineralisation, for example.

To date there is no effective and approved medical treatment available which is based on the inhibition of ENPP1.

Furthermore, ENPP1 hydrolyses cGAMP, suppressing STING-mediated innate immunity. Therefore, ENPP1 inhibitors may provide the potential to restore anti-tumour immunity in cold tumours and expand responses to immune checkpoint blockade via endogenous STING activation.

ENPP1 expression is high in many tumours and surrounding immune cells and associated with poor prognosis and metastases (e.g. breast, lung and ovarian tumours). For example, ENPP1 expression correlates with low immune cell infiltration and resistance to anti-PD-1/PD-L1 in breast cancers. Therefore, there is an unmet need for a highly selective and potent ENPP1 inhibitor in tumours with otherwise poor response to immune checkpoint therapies (Li et al., 2021: Cancer Discov, 2021 May; 11 (5): 1212-1227-Metastasis and immune evasion from extracellular cGAMP hydrolysis; Wang et al., 2021; Hu et al., 2019: Am J Cancer Res, 2019 Jan. 1; 9 (1): 134-144-Dysregulated ENPP1 increases the malignancy of human lung cancer by inducing epithelial-mesenchymal transition phenotypes and stem cell features; Abbasi et al., 2011: Hybridoma (Larchmt). 2011 February; 30 (1): 11-7-Characterisation of monoclonal antibodies to the plasma cell alloantigen ENPP1).

The present invention has been devised with the above observations in mind.

### SUMMARY OF THE INVENTION

In one aspect of the invention there is provided a compound of formula (I):

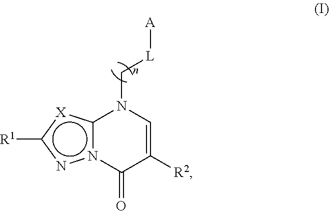

or a pharmaceutically acceptable salt or solvate thereof, or combinations thereof, wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having one or more (e.g. 1, 2, 3, or 4) ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   L is selected from phenyl, 6-membered heteroaryl having one or more (e.g. 1, 2, 3, or 4) ring heteroatoms independently selected from N, O, and S, C3-C7 cycloalkyl, 3-7-membered heterocycloalkyl having one or more (e.g. 1, 2, 3, or 4) ring heteroatoms independently selected from N, O, and S, C9-C10 bicyclic aryl, and 9-10-membered bicyclic heteroaryl having one or more (e.g. 1, 2, 3, or 4) ring heteroatoms independently selected from N, O, and S, wherein each phenyl, heteroaryl, cycloalkyl, heterocycloalkyl, bicyclic aryl or bicyclic heteroaryl is optionally substituted with one or more (e.g., 1, 2, 3, 4, or 5) substituents independently selected from R6 and R7;
   each R6 and R7 are independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me;
   each R11 is independently selected from H and C1-C3 alkyl;
   A is selected from —SO₂NH₂ and —S(O)(NH)R13;
   R13 is C1-C3 alkyl, C1-C3 haloalkyl, or C3-C6 cycloalkyl, wherein cycloalkyl is optionally substituted with one or more (e.g., 1, 2, 3, or 4) halo; or
   when L is phenyl or heteroaryl, R13 and R6 or R7 together form a 5-6 membered heterocycloalkyl optionally having one or more (e.g., 1, 2, or 3) additional ring heteroatoms independently selected from N, S and O.

In another aspect there is provided a compound selected from the group of compounds shown in Table 1. In embodiments, the compound may be selected from the group of compounds of Table 1 having a pEC₅₀ for ENPP1 human enzyme of >5, >6, >7 or >8. In other aspects and embodiments, the compound may be selected from any one of the compounds of Examples 1 to 73.

In another aspect the invention provides a pharmaceutical composition comprising a compound according to this disclosure.

In yet another aspect there is provided a method of treating or preventing mineralisation, calcium handling or calcification-related diseases or disorders, such as hypophosphatasia (HPP), in a subject. The method may comprise administering to a subject in need thereof an effective amount of the compound or pharmaceutical composition according to this disclosure.

In another aspect there is provided a compound according to this disclosure, or a pharmaceutical composition according to this disclosure for use in the treatment of mineralisation, calcium handling or calcification-related diseases, conditions or disorders. For example, the compounds and pharmaceutical compositions of the disclosure may be for use in treating a disorder or disease selected from the group consisting of: hypophosphatasia (HPP) and cancers.

Within the scope of this disclosure, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. More particularly, it is specifically intended that any embodiment of any aspect may form an embodiment of any other aspect, and all such combinations are encompassed within the scope of the invention. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE FIGURES

FIG. 1 includes two graphs showing the diet schedule and body weight of the control and Compound 11 treated mice as they age.

FIG. 2 is a series of graphs showing the plasma chemistry of wild-type (WT), control, and Compound 11 treated male mice.

FIG. 3 is a series of graphs showing plasma levels of AP, OPN and PPi (including plasma PPi vs. Compound 11) after 100 days of dosing.

FIG. 4A is a series of X-ray images of the knee joints of both male and female mice.

FIG. 4B shows the results of a bone morphometric analysis (2D analysis of trabecular bones) which was performed on Von Kossa-stained sections using Kawamoto's film method.

FIG. 5 shows a schematic diagram of Alpl allele exon and PCR primer alignment for detection of Alpl gene knockout and genotyping of Alp/knockout mice. In the presence of exons 3 and 4 a 1,837 bp PCR product is synthesised from primers 1 and 2 and in the absence of exons 3 and 4 a 716 bp product is formed. Meanwhile, in the presence of exons 3 and 4 a 912 bp PCR product is synthesised from primers 3 and 4 and in the absence of exons 3 and 4 no product is formed.

### DETAILED DESCRIPTION

Described herein are compounds and compositions (e.g. organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from ENPP1 inhibition include, for example, hypophosphatasia (HPP) and cancers.

The disclosure also encompasses salts, solvates, tautomers and functional derivatives of the compounds described herein. These compounds may be useful in the treatment of diseases or disorders which may benefit from ENPP1 inhibition, such as hypophosphatasia (HPP) and cancers.

This invention addresses some of the challenges faced by existing HPP treatments.

In particular, this invention provides an affordable and accessible treatment as the compounds disclosed herein may be orally available, allowing for oral administration which is a more tolerable route of administration. In addition, the non-immunogenic small molecule approach which is the subject of this invention offers a safe solution in HPP patients with residual tissue-nonspecific alkaline phosphatase (TNAP) activity.

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present invention employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety.

Prior to setting forth the detailed description of the invention, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising one or more compound of the disclosure.

The term ‘compound’ as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified. The term ‘tautomer’ as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

It will be appreciated that certain compounds provided herein may contain one or more centres of asymmetry and may therefore be prepared and isolated in a mixture of isomers such as a racemic mixture, or in an enantiomerically pure form.

In some embodiments the stereochemistry of compounds containing one or more centres of asymmetry is not specified. In some such embodiments having more than one centre of asymmetry, the stereochemistry of one or more centres of asymmetry may not be specified, whilst the stereochemistry of one or more other centres of asymmetry may be specified. In other such embodiments having more than one centre of asymmetry, the stereochemistry may not be specified for any centres of asymmetry. In embodiments where the stereochemistry of one or more centres of asymmetry is not specified, it is intended to encompass all possible combinations of stereoisomers (around such centres of asymmetry where the stereochemistry is not specified), including non-racemic mixtures of stereoisomers, racemic mixtures of stereoisomers, single diastereomers etc. In embodiments where the stereochemistry around one or more centres of asymmetry is not specified, all bonds around such centre(s) will be represented by solid straight lines, e.g. [symbol] and thus, all possible enantiomers and diastereoisomers are encompassed.

In some embodiments the stereochemistry of compounds containing one or more centres of asymmetry is specified as a single diastereomer. In some such embodiments having more than one centre of asymmetry, the stereochemistry of one or more centres of asymmetry may be specified as a single stereoisomer, whilst the stereochemistry of one or more other centres of asymmetry may not be specified. In other such embodiments having more than one centre of asymmetry, the stereochemistry may be specified for all centres of asymmetry. In embodiments where the stereochemistry of one or more centres of asymmetry is specified, it is intended to encompass a single stereoisomer (around such centres of asymmetry where the stereochemistry is specified) only. In embodiments where the stereochemistry around one or more centres of asymmetry is specified as a single stereoisomer, one or more bonds around such centre(s) will be represented by a hashed wedge, e.g. [symbol] or a bold wedge, e.g. [symbol] . However, unless otherwise stated, the specific form of the stereoisomer may not have been resolved and so the exact stereochemistry may have been arbitrarily defined.

In some embodiments it is intended to specify a racemic mixture of stereoisomers or diastereomers around one or more centre of asymmetry. In some such embodiments having more than one centre of asymmetry, it may be specified that a racemic mixture around one or more centre of symmetry is present, whilst other centres of asymmetry are not necessarily present in a racemic mixture (e.g. the stereochemistry for such other centres of asymmetry is not specified or is specified as a single stereoisomer). In other such embodiments having more than one centre of asymmetry, it may be specified that a racemic mixture is present around all centres of symmetry. In embodiments where the stereochemistry around one or more centres of asymmetry is specified as a racemic mixture, one or more bonds around such centre(s) will be represented by a wavy line, e.g. [symbol] .

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen (H) is mentioned (e.g. in C1-C3 alkyl), it is understood to refer to ¹H, ²H, ³H or mixtures thereof; when carbon (C) is mentioned, it is understood to refer to ¹¹C, ¹²C, ¹³C, ¹⁴C or mixtures thereof; when nitrogen (N) is mentioned, it is understood to refer to ¹³N, ¹⁴N, ¹⁵N or mixtures thereof; when oxygen (O) is mentioned, it is understood to refer to ¹⁴O, ¹⁵O, ¹⁶O, ¹⁷O, ¹⁸O or mixtures thereof; and when fluoro (F) is mentioned, it is understood to refer to ¹⁸F, ¹⁹F or mixtures thereof; unless expressly noted otherwise. For example, in deuteroalkyl and deuteroalkoxy groups, where one or more ¹H atoms are specifically replaced with deuterium (²H or D). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabelled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

Solvates of the compounds of the disclosure are also encompassed within the scope of the disclosure.

In the context of the present disclosure, the terms ‘individual’, ‘subject’, or ‘patient’ are used interchangeably to indicate an animal that may be suffering from a medical (pathological) condition and may be responsive to a molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

As used herein, terms ‘treat’, ‘treating’ or ‘treatment’ refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. ‘Treatment’ can also mean prolonging survival as compared to expected survival if not receiving treatment.

The terms ‘prevent’, ‘preventing’ or ‘prevention’ as used herein means the prevention of the onset, recurrence or spread, in whole or in part, of the disease or condition as described herein, or a symptom thereof.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-C6 alkyl (or C1-6 alkyl) refers to any alkyl group containing 1 to 6 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. In some cases, common abbreviations for alkyl groups/radicals may be used, including but not limited to ‘Me’ for methyl, ‘Et’ for ethyl, ‘Pr’ for propyl, ‘iPr’ for isopropyl, ‘Bu’ for butyl, ‘tBu’ for tert-butyl. Optional substituents for alkyl and other groups are described below.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this invention and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. By ‘optionally substituted’ it is meant that the group concerned is either unsubstituted, or at least one hydrogen atom is replaced with one of the specified substituent groups, radicals or moieties.

Any radical/group/moiety described herein that may be substituted (or optionally substituted) may be substituted with one or more (e.g. one, two, three, four or five) substituents, which are independently selected from the designated group of substituents. Thus, substituents may be selected from the group: halogen (or ‘halo’, e.g. F, Cl and Br), hydroxyl (—OH), amino or aminyl (—NH₂), thiol (—SH), cyano (—CN), (lower) alkyl, (lower) alkoxy, (lower) alkenyl, (lower) alkynyl, aryl, heteroaryl, (lower) alkylthio, oxo, haloalkyl, hydroxyalkyl, nitro (—NO₂), phosphate, azido (—N₃), alkoxycarbonyl, carboxy, alkylcarboxy, alkylamino, dialkylamino, aminoalkyl, alkylaminoalkyl, dialkylaminoalkyl, thioalkyl, alkylsulfonyl, arylsulfinyl, alkylaminosulfonyl, arylaminosulfonyl, alkylsulfonylamino, arylsulfonylamino, carbamoyl, alkylcarbamoyl, dialkylcarbamoyl, arylcarbamoyl, alkylcarbonylamino, arylcarbonylamino, cycloalkyl, heterocycloalkyl, unless otherwise indicated. Alternatively, where the substituents are on an aryl or other cyclic ring system, two adjacent atoms may be substituted with a methylenedioxy or ethylenedioxy group. More suitably, the substituents are selected from: halogen, hydroxy, amino, thiol, cyano, (C₁-C₆)alkyl, (C₁-C₆)alkoxy, (C₁-C₆)alkenyl, (C₁-C₆)alkynyl, aryl, aryl(C₁-C₆)alkyl, aryl(C₁-C₆)alkoxy, heteroaryl, (C₁-C₆)alkylthio, oxo, halo(C₁-C₆)alkyl, hydroxy(C₁-C₆)alkyl, nitro, phosphate, azido, (C₁-C₆)alkoxycarbonyl, carboxy, (C₁-C₆)alkylcarboxy, (C₁-C₆)alkylamino, di(C₁-C₆)alkylamino, amino(C₁-C₆)alkyl, (C₁-C₆)alkylamino(C₁-C₆)alkyl, di(C₁-C₆)alkylamino(C₁-C₆)alkyl, thio(C₁-C₆)alkyl, (C₁-C₆)alkylsulfonyl, arylsulfinyl, (C₁-C₆)alkylaminosulfonyl, arylaminosulfonyl, (C₁-C₆)alkylsulfonylamino, arylsulfonylamino, carbamoyl, (C₁-C₆)alkylcarbamoyl, di(C₁-C₆)alkylcarbamoyl, arylcarbamoyl, (C₁-C₆)alkylcarbonylamino, arylcarbonylamino, (C₁-C₆) cycloalkyl, and heterocycloalkyl. Still more suitably, the substituents are selected from one or more of: fluoro, chloro, bromo, hydroxy, (C₁-C₆)alkyl, (C₁-C₆)haloalkyl, (C₁-C₆)alkoxy, (C₅-C₆)aryl, a 5- or 6-membered heteroaryl, (C₄-C₆)cycloalkyl, a 4- to 6-membered heterocycloalkyl, cyano, (C₁-C₆)alkylthio, amino, —NH(alkyl), —NH((C₁-C₆) cycloalkyl), —N((C₁-C₆)alkyl)₂, —OC(O)—(C₁-C₆)alkyl, —OC(O)—(C₅-C₆) aryl, —OC(O)—(C₁-C₆) cycloalkyl, carboxy and —C(O)O—(C₁-C₆)alkyl. Most suitably, the substituents are selected from one or more of: fluoro, chloro, bromo, hydroxy, amino, (C₁-C₆)alkyl and (C₁-C₆)alkoxy, wherein alkyl and alkoxy are optionally substituted by one or more chloro. Particularly preferred substituents are: chloro, methyl, ethyl, methoxy and ethoxy.

The term ‘halo’ refers to one of the halogen radical/substituent from group 17 of the periodic table. In particular, the term refers to fluorine, chlorine, bromine and iodine. Particularly suitable halo groups are fluorine and chlorine. A ‘halogenated’ compound is one substituted with one or more halo substituent.

As used herein, the term ‘cyano’ refers to a —CN radical. As used herein, the term ‘hydroxyl’ refers to an —OH radical. As used herein, the term ‘amino’ refers to an —NH₂ group. As used herein, the term ‘oxo’ refers to an ‘═O’ group attached to a carbon atom.

The term ‘C₁-C₆ haloalkyl’ or ‘C1-C6 haloalkyl’ refers to a hydrocarbon chain substituted with at least one halogen atom independently chosen at each occurrence, for example fluorine, chlorine, bromine and iodine, and up to a perhalogenated alkyl (i.e., each hydrogen atom of the alkyl is replaced with a halo atom). The halogen atom may be present at any position on the hydrocarbon chain. Similarly, a C1-C3 haloalkyl group is linear or branched hydrocarbon chain containing 1, 2, or 3 carbon atoms substituted with at least one halogen atom. For example, C1-C3 haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

As used herein, the term ‘geminal’ refers to substituent atoms or groups attached to the same atom in a molecule. As used herein, the term ‘vicinal’ refers to substituent atoms or groups attached to adjacent atoms in a molecule. The stereochemical relationship between the substituent atoms or groups can be cis, trans, undefined, or unresolved.

When used herein, the term ‘independently’, in reference to the substitution of a parent moiety with one or more substituents, means that the parent moiety may be substituted with any of the listed substituents, either individually or in combination, and any number of chemically possible substituents may be used. In any of the embodiments, where a group is substituted, it may contain up to 5, up to 4, up to 3, 2 or 1 substituent.

‘Alkylene’ or ‘alkylenyl’ means a difunctional group obtained by removal of a hydrogen atom from an alkyl group as defined above. Non-limiting examples of alkylene include methylene, ethylene and propylene. ‘Lower alkylene’ means an alkylene having from 1 to 6 carbon atoms in the chain, and may be straight or branched. Alkylene groups are optionally substituted.

The term ‘alkenyl’ refers to a monovalent, optionally substituted, unsaturated aliphatic hydrocarbon radical. Therefore, an alkenyl has at least one carbon-carbon double bond (C═C). The number of carbon atoms in the alkenyl group may be indicated, such as from 2 to about 20. For example, a C2-12 alkenyl (or C2-12 alkenyl) refers to an alkenyl group containing 2 to 12 carbon atoms in the structure. Alkenyl groups may be straight (i.e. linear), branched chain, or cyclic. ‘Lower alkenyl’ refers to an alkenyl of 1 to 6 carbon atoms, and may have from 1 to 4 carbon atoms, or 1 to 2 carbon atoms. Representative examples of lower alkenyl radicals include ethenyl, 1-propenyl, 1-butenyl, 1-pentenyl, 1-hexenyl, isopropenyl, isobutenyl, and the like. Higher alkenyl refers to alkenyls of seven carbons and above, such as 1-heptenyl, 1-octenyl, 1-nonenyl, 1-decenyl, 1-dodecenyl, 1-tetradecenyl, 1-hexadecenyl, 1-octadecenyl, 1-eicosenyl, and the like, along with branched variations thereof. Optional substituents include are described elsewhere.

‘Alkenylene’ means a difunctional group obtained by removal of a hydrogen from an alkenyl group that is defined above. Non-limiting examples of alkenylene include —CH═CH—, —C(CH₃)═CH—, and —CH═CHCH₂—.

‘Alkynyl’ and ‘lower alkynyl’ is defined similarly to the term ‘alkenyl’, except that it includes at least one carbon-carbon triple bond.

The term ‘alkoxy’ or ‘alkoxyl’ refers to a monovalent radical of the formula RO—, where R is any alkyl, alkenyl or alkynyl as defined herein. Alkoxy groups may be optionally substituted by any of the optional substituents described herein, e.g., haloalkoxyl refers to an alkoxyl group substituted with one or more halo (up to and including a perhalogenated alkoxyl). ‘Lower alkoxy’ has the formula RO—, where the R group is a lower alkyl, alkenyl or alkynyl. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Preferred alkoxy groups are methoxy and ethoxy.

The term ‘aryl’ as used herein refers to a substituted or unsubstituted aromatic carbocyclic radical containing from 6 to about 15 carbon atoms. An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxy’ group, therefore, is a radical of the formula RO—, wherein R is a phenyl radical. ‘Benzyl’ is a radical of the formula R—CH₂—, wherein R is phenyl, and ‘benzyloxy’ is a radical of the formula RO—, wherein R is benzyl. Non-limiting examples of aryl radicals include, phenyl, naphthyl, benzyl, biphenyl, indanyl, anthracenyl, tetrahydronaphthyl, a benzoic acid radical, and the like.

A ‘heteroaryl’ group is herein defined as a substituted or unsubstituted ‘aryl’ group in which one or more carbon atoms in the ring structure has been replaced with a heteroatom, such as nitrogen, oxygen or sulphur. Generally, the heteroaryl group contains one or two heteroatoms. Particularly suitable heteroatoms are N and O; and a preferred heteroatom is N. Exemplary heteroaryl groups include: furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinazoline, pyridazine and cinnoline.

The terms ‘heterocycle’, ‘heterocyclic’ or ‘heterocycloalkyl’ group as used herein refer to a monovalent radical of from about 4- to about 15-ring atoms, and preferably 4-, 5-, 6- or 7-ring members. Generally, the heterocyclic group contains one, two or three heteroatoms, selected independently from nitrogen, oxygen and sulphur. Particularly suitable heteroatoms are N and O; and a preferred heteroatom is N. A heterocyclic group may have only one individual ring, or may comprise one or more fused rings in which at least one ring contains a heteroatom. It may be fully saturated or partially saturated, and may be substituted or unsubstituted as in the case or aryl and heteroaryl groups. Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocycles with only one heteroatom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity or through a linker such as an alkylene such as methylene or ethylene.

Typically, herein, the number of atom members in a heterocyclic ring system is stated as, e.g. “an X to Y-membered heterocyclic ring” or “an X to Y-membered heterocycloalkyl”, where X and Y indicate the lower and upper range for the number of atoms forming the aromatic or cyclic system.

The term ‘pharmaceutically acceptable’ indicates that the compound, or salt or composition thereof is compatible chemically and/or toxicologically with the other ingredients comprising a formulation and/or the subject being treated therewith.

Unless defined otherwise, ‘room temperature’ is intended to mean a temperature of from about 18 to 28° C., typically between about 18 and 25° C., and more typically between about 18 and 22° C. As used herein, the phrase ‘room temperature’ may be shortened to ‘rt’ or ‘RT’.

### Molecules and Compounds

In aspects and embodiments, the compounds of the disclosure are amide compounds.

Disclosed herein is a compound having the structural formula (I) below, or a pharmaceutically acceptable salt or solvate thereof. It should be appreciated that each possible tautomer of the structures of the formulae (I) are encompassed within the present disclosure, as further defined herein.

Accordingly in one aspect, there is provided a compound having the structural formula (I) or a pharmaceutically acceptable salt or solvate thereof:

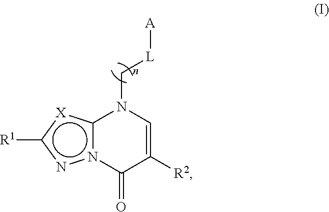

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having one or more (e.g. 1, 2, 3, or 4) ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   L is selected from phenyl, 6-membered heteroaryl having one or more (e.g. 1, 2, 3, or 4) ring heteroatoms independently selected from N, O, and S, C3-C7 cycloalkyl, 3-7-membered heterocycloalkyl having one or more (e.g. 1, 2, 3, or 4) ring heteroatoms independently selected from N, O, and S, C9-C10 bicyclic aryl, and 9-10-membered bicyclic heteroaryl having one or more (e.g. 1, 2, 3, or 4) ring heteroatoms independently selected from N, O, and S, wherein each phenyl, heteroaryl, cycloalkyl, heterocycloalkyl, bicyclic aryl or bicyclic heteroaryl is optionally substituted with one or more (e.g., 1, 2, 3, 4, or 5) substituents independently selected from R6 and R7;
   each R6 and R7 are independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me;
   each R11 is independently selected from H and C1-C3 alkyl;
   A is selected from —SO₂NH₂ and —S(O)(NH)R13;
   R13 is C1-C3 alkyl, C1-C3 haloalkyl, or C3-C6 cycloalkyl, wherein cycloalkyl is optionally substituted with one or more (e.g., 1, 2, 3, or 4) halo; or
   when L is phenyl or heteroaryl, R13 and R6 or R7 together form a 5-6 membered heterocycloalkyl optionally having one or more (e.g., 1, 2, or 3) additional ring heteroatoms independently selected from N, S and O.

As indicated above, in any aspects and embodiments, where R¹, R², L and A are described as above each possible tautomer of the structures of the formula (I) are encompassed. For example, the following tautomers of structure (I) are encompassed within the scope of this disclosure, and X is selected accordingly:

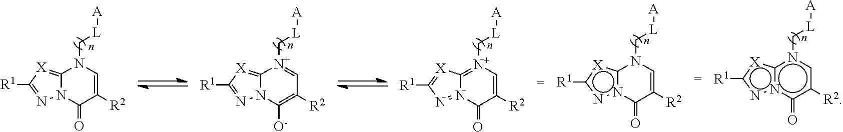

In some embodiments X is N. In some embodiments X is C(CH₂)ₘR5, wherein m and R5 are as defined above.

In particular embodiments, the following tautomers are encompassed:

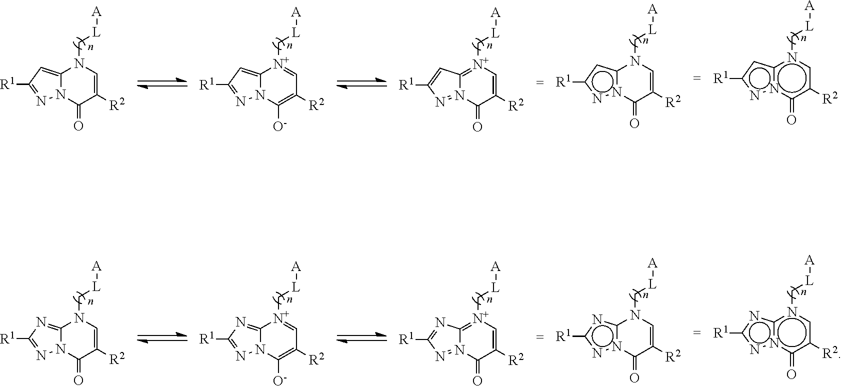

In some embodiments, X is N. In other embodiments, X is CR5, wherein R5 is H (e.g. ¹H or ²H) or halo.

In some embodiments, R1 is H. In some embodiments, R1 is H or is selected from:

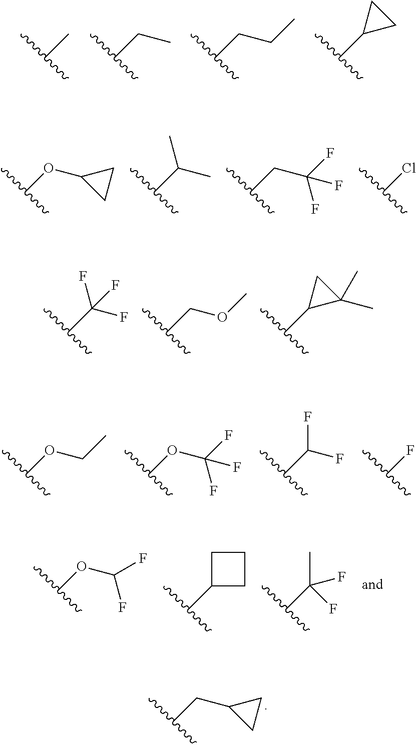

In some embodiments, R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

In some embodiments, R2 is selected from:

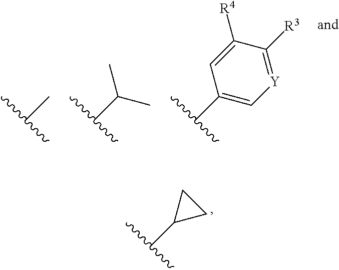

wherein Y is CH or N; R3 is H or halo; and R4 is H or halo, and halo is optionally F.

In some embodiments, R6 and/or R7 is NHMe, NH₂ or NMe₂.

In some embodiments, L-A is selected from;

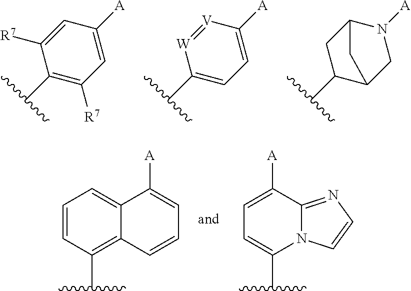

wherein
   V is CR6 or N;
   W is CR7 or N; and
   each R6 and R7 is independently selected from H, halo, hydroxy, C1-C4 alkoxy, C1-C4 haloalkoxyl, C1-C6 alkyl, C1-C6 haloalkyl, C1-C3 alkoxyl-C1-C3 alkyl, phenyl, 6-membered heteroaryl having one or more (e.g. 1, 2, 3, or 4) ring heteroatoms independently selected from N, O, and S, —NH₂, —NHMe, and —NMe₂, wherein each phenyl or heteroaryl is optionally substituted with one or more (e.g., 1, 2, 3, or 4) halo.

In certain embodiments, wherein L-A is as defined above, R6 is selected from H, halo and hydroxy.

In certain embodiments, wherein L-A is as defined above, R6 is H.

In certain embodiments, wherein L-A is as defined above, R6 is H or is selected from:

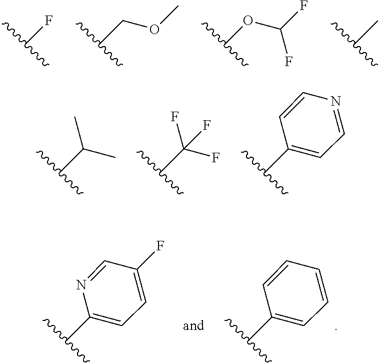

In some embodiments, n is 1.

In some embodiments, L-A is:

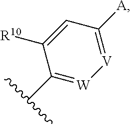

wherein
V is N or CR6;
W is N or CR7;
R6 is H, halo, hydroxy, C1-C4 alkyl, C1-C4 haloalkyl, C1-C4 alkoxyl, C1-C4 haloalkoxyl, C1-C3 alkoxyl-C1-C3 alkyl, phenyl, or 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R7 is H, C1-C4 alkyl, hydroxy, —N(H)(R11), or halo;
R10 is H or halo; and
R11 is H or C1-C3 alkyl.

In some embodiments L-A is selected from:

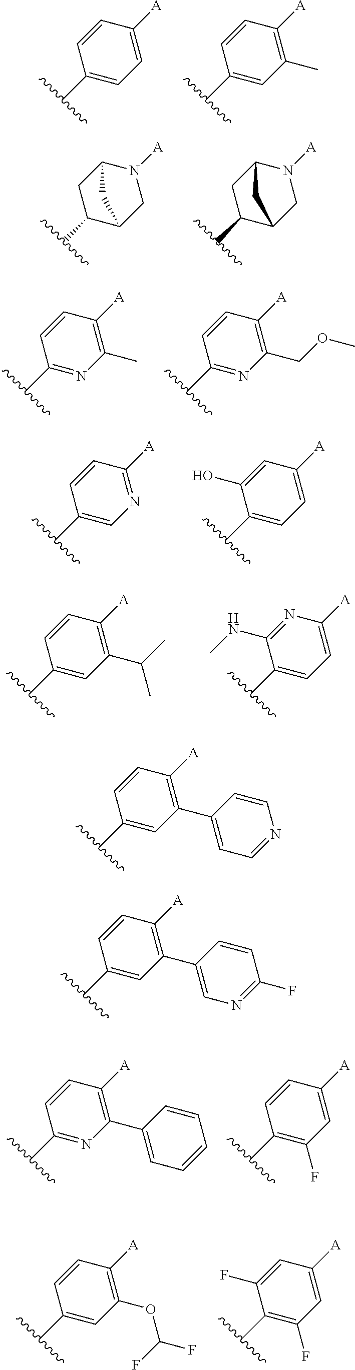

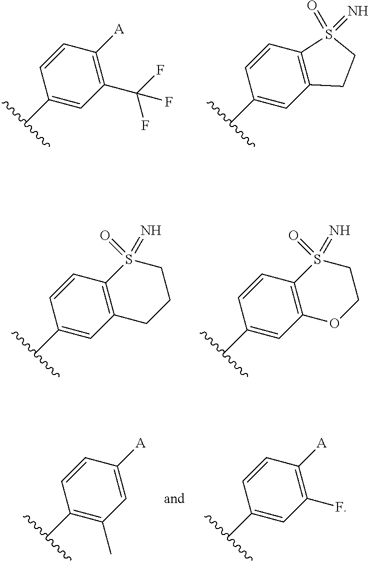

In some embodiments, L-A is selected from:

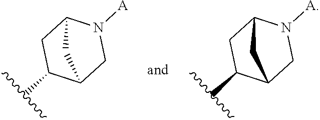

In some embodiments, L-A is selected from:

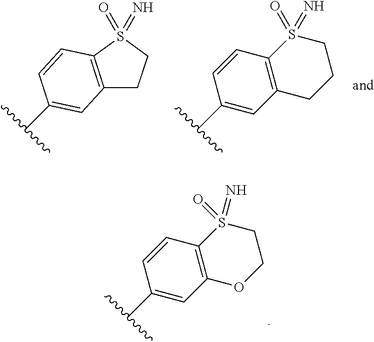 [SMILES: *c1ccc2c(c1)CCS2(=N)=O ; *c1ccc2c(c1)CCCS2(=N)=O ; *c1ccc2c(c1)OCCS2(=N)=O]

In some embodiments, L-A is selected from:

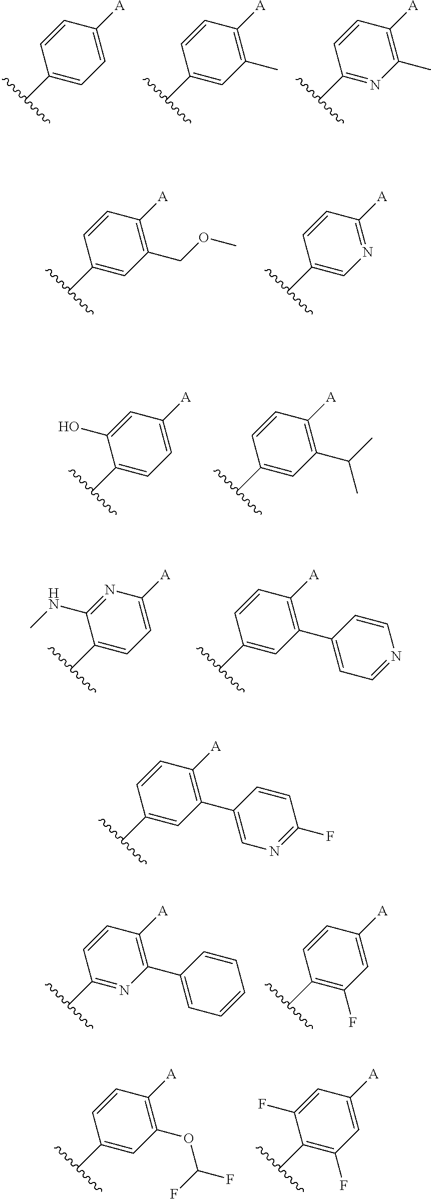

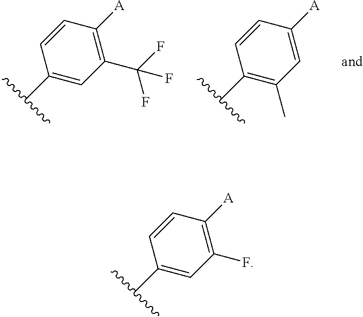

In some embodiments, A is —SO₂NH₂.

In some embodiments, A is —S(O)(NH)R13, wherein R13 is C1-C3 alkyl (e.g. C¹H₃ or C²H₃), C1-C3 haloalkyl, or C3-C6 cycloalkyl, and cycloalkyl is optionally substituted with one or more (e.g., 1, 2, 3, or 4) halo.

In some embodiments, A is selected from:

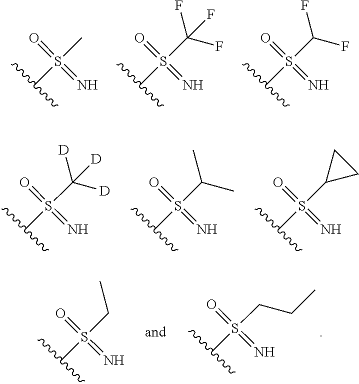

In some embodiments, A is —S(O)(NH) Me.

In some embodiments, A is selected from:

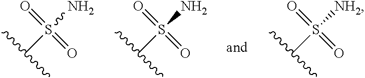

wherein [symbol] indicates a racemic mixture

In some embodiments, A is selected from:

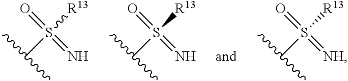

wherein [symbol] indicates a racemic mixture and R13 is as defined above.

In some embodiments, the invention provides a compound of formula (II) or a pharmaceutically acceptable salt or solvate thereof:

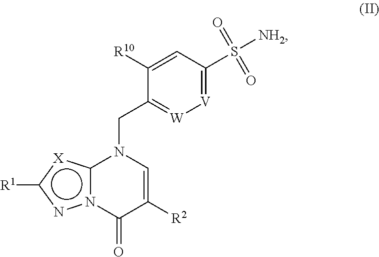

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   V is N or CR6;
   W is N or CR7;
   each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
   R10 is H or halo; and
   R11 is H or C1-C3 alkyl.

In some embodiments, the invention provides a compound of formula (III) or a pharmaceutically acceptable salt or solvate thereof:

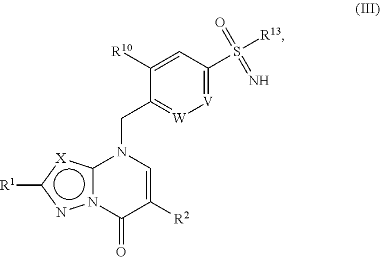

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   V is N or CR6;
   W is N or CR7;
   each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
   R10 is H or halo;
   R11 is H or C1-C3 alkyl; and
   R13 is C1-C3 alkyl, C1-C3 haloalkyl, or C3-C6 cycloalkyl, wherein cycloalkyl is optionally substituted with one or more (e.g., 1, 2, 3, or 4) halo; or
   R13 and R6 together form a 5-6 membered heterocycloalkyl optionally having one or more (e.g., 1, 2 or 3) additional ring heteroatoms independently selected from N, S and O.

In some embodiments, the invention provides a compound of formula (IV) or (V) or a pharmaceutically acceptable salt or solvate thereof:

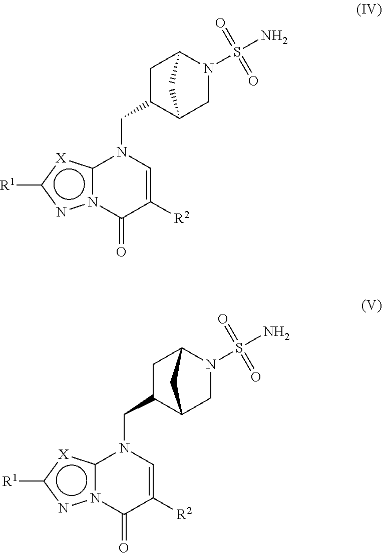

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo; and
   m is 0 or 1.

In some embodiments, the invention provides a compound of formula (VI) or (VII) or a pharmaceutically acceptable salt or solvate thereof:

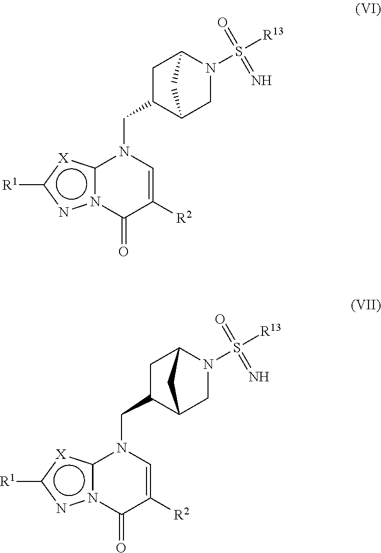

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   m is 0 or 1; and
   R13 is C1-C3 alkyl, C1-C3 haloalkyl, or C3-C6 cycloalkyl, wherein cycloalkyl is optionally substituted with one or more (e.g., 1, 2, 3, or 4) halo.

In some embodiments, the invention provides a compound of formula (I) or a pharmaceutically acceptable salt or solvate thereof:

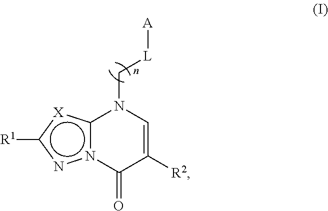

wherein:
   X is N or CH;
   R1 is selected from C1-C6 alkyl, C1-C6 haloalkyl and C3-C6 cycloalkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo;
   R2 is selected from C1-C6 alkyl and C1-C6 haloalkyl;
   n is 1, 2, or 3;
   L is phenyl, wherein the phenyl is optionally substituted with one or more (e.g., 1, 2, 3, or 4) substituents independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S;
   each R11 is independently selected from H and C1-C3 alkyl;
   A is selected from —SO₂NH₂ and —S(O)(NH)R13; and
   R13 is C1-C3 alkyl, C1-C3 haloalkyl, or C3-C6 cycloalkyl, wherein cycloalkyl is optionally substituted with one or more (e.g., 1, 2, 3, or 4) halo.

In some embodiments, the invention provides a compound of formula (VIII) or a pharmaceutically acceptable salt or solvate thereof:

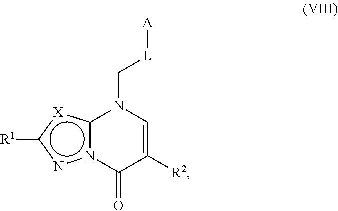

wherein:
   X is N or CH;
   R1 is selected from C1-C6 alkyl and C3-C6 cycloalkyl;
   R2 is C1-C6 alkyl;
   L is phenyl, wherein the phenyl is optionally substituted with one or more (e.g., 1, 2, 3, or 4) substituents independently selected from halo and C1-C6 alkyl;
   A is selected from —SO₂NH₂ and —S(O)(NH)R13; and
   R13 is C1-C3 alkyl or C3-C6 cycloalkyl.

In some embodiments, the invention provides a compound selected from a compound of:
(i) the group of compounds shown in Table 1;
(ii) the group of compounds of Table 1 having a pEC₅₀ for ENPP1 human enzyme of >5;
(iii) the group of compounds of Table 1 having a pEC₅₀ for ENPP1 human enzyme of >6;
(iv) the group of compounds of Table 1 having a pEC₅₀ for ENPP1 human enzyme of >7; or
(v) the group of compounds of Table 1 having a pEC₅₀ for ENPP1 human enzyme of >8;
or a pharmaceutically acceptable salt or solvate thereof.

In some embodiments, the invention provides a compound, or a pharmaceutically acceptable salt or solvate thereof, selected from the group of compounds consisting of:

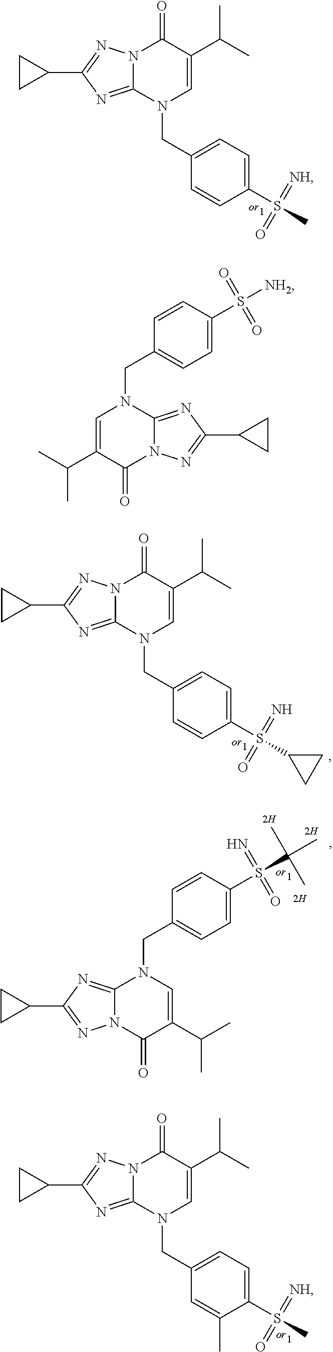

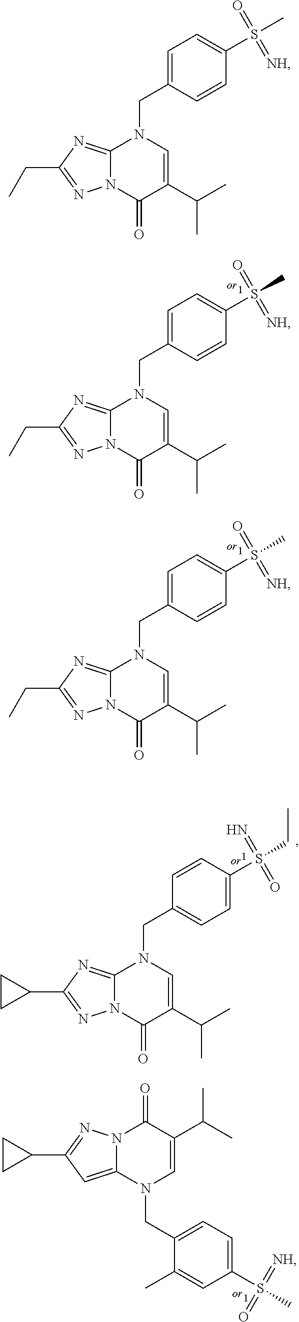

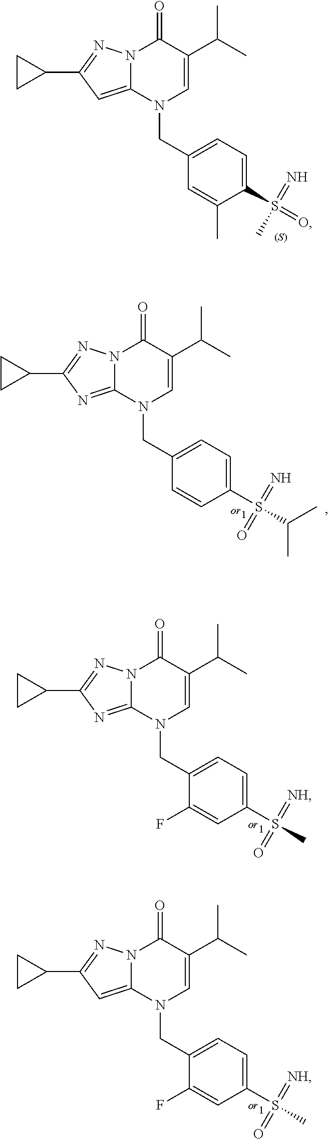

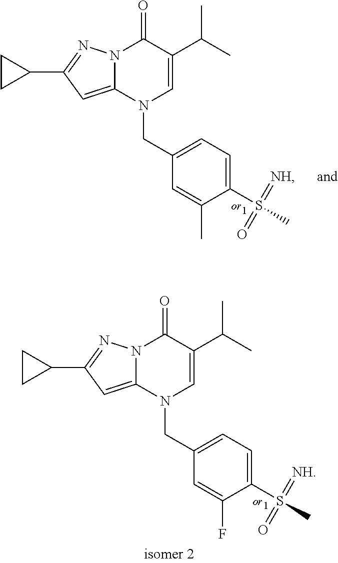 [SMILES: Cc1cc(Cn2cc(C(C)C)c(=O)n3nc(C4CC4)cc23)ccc1[S@@](C)(=N)=O ; CC(C)c1cn(Cc2ccc([S@](C)(=N)=O)c(F)c2)c2cc(C3CC3)nn2c1=O]

In some embodiments, the invention provides a pharmaceutical composition comprising a compound as disclosed herein (e.g., a compound of formula (I), (II), (III), (IV) (V), (VI), (VII), or (VIII) or a compound of Table 1), or a pharmaceutically acceptable salt or solvate thereof, and one or more pharmaceutically acceptable carrier.

In some embodiments, the invention provides a compound or a pharmaceutical composition as defined above, for use in the treatment of a disease or disorder.

In some embodiments, the invention provides a compound or a pharmaceutical composition for use, as defined above, wherein the disease or disorder is a calcium handling or calcification-related disease or disorder.

In some embodiments, the invention provides a compound or a pharmaceutical composition for use, as defined above, wherein the disease or disorder is selected from the group consisting of: hypophosphatasia (HPP) and cancers.

In some embodiments, the invention provides a compound or pharmaceutical composition as defined above, a compound or pharmaceutical composition for use as defined above, wherein the compound is an inhibitor of ENPP1.

In some embodiments, the invention provides a compound or pharmaceutical composition for use as defined above, wherein the use is in a method comprising administering the compound according to any of formulas (I) to (VIII) in combination with one or more additional therapeutic agent.

In any of the above definitions of embodiments and compounds of the invention or disclosure, it is expressly intended that any broad, optional, preferred, suitable, beneficial or particular definition of any group, moiety or portion of a compound may be combined with any definition of any other group, moiety or portion of a compound, whether that other definition is broad, optional, preferred, suitable, beneficial or particular for that other group, moiety or portion of a compound.

Specific compounds according to various aspects and embodiments of this disclosure may have the structures as defined in Table 1 below.

TABLE 1
Compounds of the present disclosure. Unless otherwise stated, stereochemistry is arbitrarily
assigned.

| Compound | Structure | Characterisation Data |
|---|---|---|
| 1 | 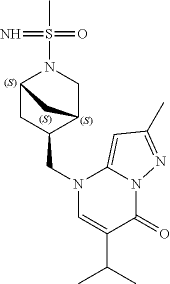 | ESI-MS m/z = 378.15 [M + H]+; Calculated: 377.2. 1H NMR (400 MHz, DMSO-d6) δ 7.76 (s, 1H), 6.21 (s, 1H), 4.09- 3.75 (m, 3H), 3.18-2.94 (m, 2H), 2.81-2.68 (m, 4H), 2.36-2.23 (m, 5H), 1.87-1.72 (m, 2H), 1.57- 1.44 (m, 1H), 1.30-1.22 (m, 1H), 1.18 (d, J = 6.9 Hz, 6H). |
| 2 | 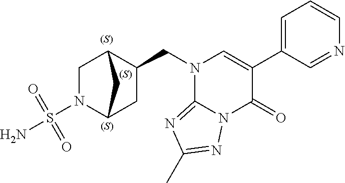 | ESI-MS m/z = 416.15 [M + H]⁺; Calculated: 415.1. ¹H NMR (400 MHz, DMSO-d₆) δ 8.84 (d, J = 2.3 Hz, 1H), 8.60- 8.52 (m, 2H), 8.13-8.06 (m, 1H), 7.52-7.45 (m, 1H), 6.68 (br, 2H), 4.20-4.11 (m, 1H), 4.06-3.98 (m, 1H), 3.95 (s, 1H), 3.12-3.05 (m, 1H), 2.74 (d, J = 9.1 Hz, 1H), 2.48-2.38 (m, 5H), 1.96-1.85 (m, 1H), 1.72-1.57 (m, 2H), 1.37- 1.27 (m, 1H). |
| 3 Rac | 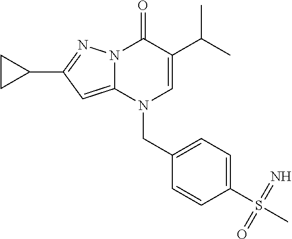 [SMILES: CC(C)c1cn(Cc2ccc(S(C)(=N)=O)cc2)c2cc(C3CC3)nn2c1=O] | ESI-MS m/z = 385.05 [M + H]⁺; Calculated:384.2. ¹H NMR (400 MHz, DMSO-d₆) δ 7.98-7.88 (m, 3H), 7.56-7.50 (m, 2H), 6.03 (s, 1H), 5.33 (s, 2H), 4.21 (s, 1H), 3.04 (s, 3H), 3.05- 2.94 (m, 1H), 1.99-1.88 (m, 1H), 1.23-1.17 (m, 6H), 0.98-0.87 (m, 2H), 0.78-0.70 (m, 2H). |
| 3A | 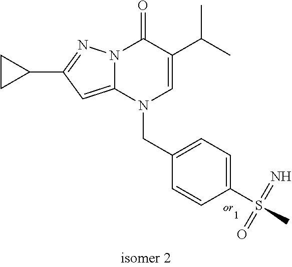 [SMILES: CC(C)c1cn(Cc2ccc([S@](C)(=N)=O)cc2)c2cc(C3CC3)nn2c1=O] | ESI-MS m/z = 385.05 [M + H]⁺; Calculated:384.2. ¹H NMR (400 MHz, DMSO-d₆) δ 7.97-7.87 (m, 3H), 7.56-7.49 (m, 2H), 6.02 (s, 1H), 5.32 (s, 2H), 4.21 (s, 1H), 3.06-2.95 (m, 4H), 1.99-1.87 (m, 1H), 1.20 (d, J = 6.9 Hz, 3H), 1.20 (d, J = 6.9 Hz, 3H), 0.98-0.87 (m, 2H), 0.78- 0.70 (m, 2H). |
| 3B | 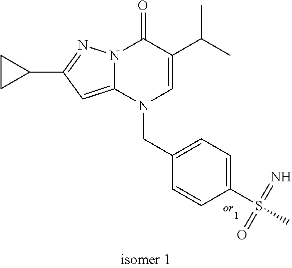 [SMILES: CC(C)c1cn(Cc2ccc([S@@](C)(=N)=O)cc2)c2cc(C3CC3)nn2c1=O] | ESI-MS m/z = 385.30 [M + H]⁺; Calculated:384.2. ¹H NMR (400 MHz, DMSO-d₆) δ 7.98-7.88 (m, 3H), 7.56-7.49 (m, 2H), 6.03 (s, 1H), 5.32 (s, 2H), 4.21 (s, 1H), 3.07-2.95 (m, 4H), 1.99-1.88 (m, 1H), 1.20 (d, J = 6.9 Hz, 3H), 1.20 (d, J = 6.9 Hz, 3H), 0.98-0.89 (m, 2H), 0.78- 0.70 (m, 2H). |
| 4 | 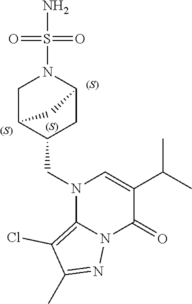 | ESI-MS m/z = 414.05 [M + H]⁺; Calculated: 413.1. ¹H NMR (400 MHz, DMSO-d6) δ 7.78 (s, 1H), 6.67 (s, 2H), 4.24- 4.08 (m, 2H), 3.95 (s, 1H), 3.09 (dd, J = 9.1, 3.5 Hz, 1H), 3.04- 2.94 (m, 1H), 2.72 (d, J = 9.1 Hz, 1H), 2.36-2.25 (m, 4H), 2.24- 2.15 (m, 1H), 1.96-1.84 (m, 1H), 1.73-1.57 (m, 2H), 1.31-1.24 (m, 1H), 1.18 (dd, J = 6.9, 2.1 Hz, 6H). |
| 5 | 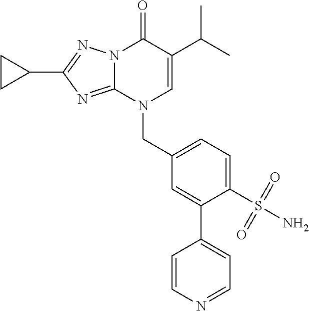 [SMILES: CC(C)c1cn(Cc2ccc(S(N)(=O)=O)c(-c3ccncc3)c2)c2nc(C3CC3)nn2c1=O] | ESI-MS m/z = 465.15 [M + H]⁺; Calculated: 464.2. ¹H NMR (400 MHz, DMSO-d₆) δ 8.65-8.56 (m, 2H), 8.04 (s, 1H), 8.00 (d, J = 8.2 Hz, 1H), 7.59 (dd, J = 8.3, 1.9 Hz, 1H), 7.48-7.42 (m, 3H), 7.39-7.34 (m, 2H), 5.39 (s, 2H), 2.98-2.90 (m, 1H), 2.09- 1.95 (m, 1H), 1.18 (d, J = 6.9 Hz, 6H), 0.92 (m, 4H). |
| 6 | 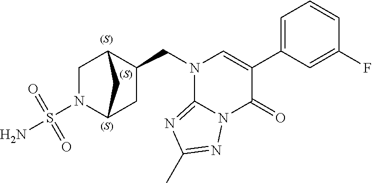 | ESI-MS m/z = 433.10 [M + H]⁺; Calculated: 432.1. ¹H NMR (400 MHz, DMSO-d₆) δ 8.53 (s, 1H), 7.60-7.45 (m, 3H), 7.25-7.16 (m, 1H), 6.68 (s, 2H), 4.16 (dd, J = 13.8, 9.1 Hz, 1H), 4.02 (dd, J = 13.8, 6.6 Hz, 1H), 3.98-3.92 (m, 1H), 3.09 (dd, J = 9.1, 3.6 Hz, 1H), 2.74 (d, J = 9.1 Hz, 1H), 2.47-2.37 (m, 5H), 1.96- 1.86 (m, 1H), 1.69 (d, J = 10.2 Hz, 1H), 1.62 (d, J = 10.3 Hz, 1H), |
| | | 1.36-1.27 (m, 1H). |
| | | ¹⁹F NMR (377 MHz, DMSO-d₆) δ |
| | | −113.454. |
| 7A | 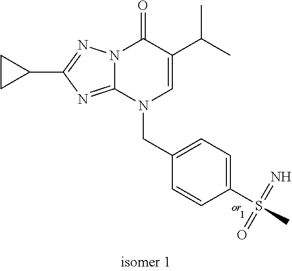 [SMILES: CC(C)c1cn(Cc2ccc([S@](C)(=N)=O)cc2)c2nc(C3CC3)nn2c1=O] | ESI-MS m/z = 386.15 [M + H]⁺; Calculated: 385.2. ¹H NMR (400 MHz, DMSO-d₆) δ 8.05-7.87 (m, 3H), 7.59-7.52 (m, 2H), 5.41 (s, 2H), 4.21 (s, 1H), 3.11-2.94 (m, 4H), 2.04 (tt, J = 8.3, 4.9 Hz, 1H), 1.19 (d, J = 6.9 Hz, 6H), 1.05-0.96 (m, 2H), 0.90 (dt, J = 4.7, 3.0 Hz, 2H). |
| 7B | 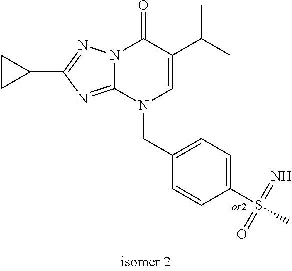 [SMILES: CC(C)c1cn(Cc2ccc([S@@](C)(=N)=O)cc2)c2nc(C3CC3)nn2c1=O] | ESI-MS m/z = 386.20. [M + H]⁺; Calculated: 385.2. ¹H NMR (400 MHz, DMSO-d₆) δ 8.11-7.88 (m, 3H), 7.60-7.53 (m, 2H), 5.41 (s, 2H), 4.54-3.82 (m, 1H), 3.12-2.90 (m, 4H), 2.10- 1.95 (m, 1H), 1.19 (d, J = 6.9 Hz, 6H), 1.04-0.93 (m, 2H), 0.90 (dt, J = 5.0, 3.0 Hz, 2H). |
| 8 | 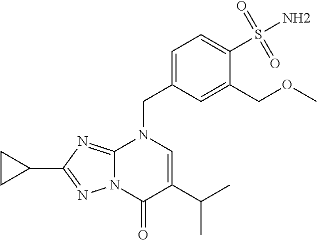 [SMILES: COCc1cc(Cn2cc(C(C)C)c(=O)n3nc(C4CC4)nc23)ccc1S(N)(=O)=O] | ESI-MS m/z = 432.05[M + H]⁺; Calculated: 431.2. ¹H NMR (400 MHz, DMSO-d₆) δ 8.07 (s, 1H), 7.82 (d, J = 8.1 Hz, 1H), 7.71 (s, 1H), 7.44 (s, 2H), 7.42 (d, J = 1.9 Hz, 1H), 5.39 (s, 2H), 4.79 (s, 2H), 3.42-3.35 (m, 3H), 3.05-2.93 (m, 1H), 2.04 (m, 1H), 1.18 (dd, J = 6.9 Hz, 6H), 1.04-0.98 (m, 2H), 0.94-0.89 (m, 2H). |
| 9 Rac | 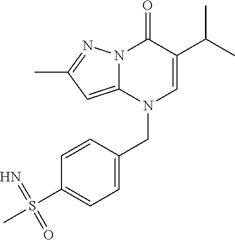 [SMILES: Cc1cc2n(Cc3ccc(S(C)(=N)=O)cc3)cc(C(C)C)c(=O)n2n1] | ESI-MS m/z = 359.10 [M + H]⁺; Calculated: 358.1. ¹H NMR (400 MHz, DMSO-d₆) δ 8.00 (s, 1H), 7.93 (d, J = 8.4 Hz, 2H), 7.54 (d, J = 8.4 Hz, 2H), 6.04 (s, 1H), 5.37 (s, 2H), 4.23 (s, 1H), 3.06 (s, 4H), 2.24 (s, 3H), 1.21 (d, J = 6.9 Hz, 6H). |
| 9A | 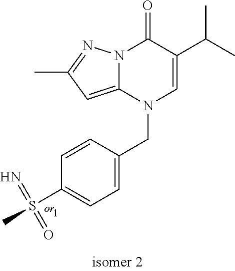 [SMILES: Cc1cc2n(Cc3ccc([S@@](C)(=N)=O)cc3)cc(C(C)C)c(=O)n2n1] | ESI-MS m/z = 359.00 [M + H]⁺; Calculated: 358.1. ¹H NMR (400 MHz, DMSO-d₆) δ 7.98 (s, 1H), 7.94-7.85 (m, 2H), 7.52 (d, J = 8.3 Hz, 2H), 6.03 (s, 1H), 5.35 (s, 2H), 4.28 (s, 1H), 3.09-2.96 (m, 4H), 2.28-2.18 (m, 3H), 1.21 (d, J = 6.8 Hz, 6H). |
| 9B | 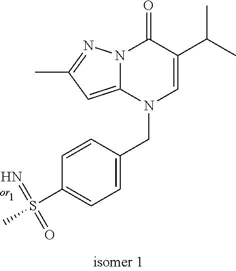 [SMILES: Cc1cc2n(Cc3ccc([S@](C)(=N)=O)cc3)cc(C(C)C)c(=O)n2n1] | ESI-MS m/z = 359.00 [M + H]⁺; Calculated: 358.1. ¹H NMR (400 MHz, DMSO-d₆) δ 8.01-7.94 (m, 1H), 7.94-7.88 (m, 2H), 7.55-7.49 (m, 2H), 6.06- 5.99 (m, 1H), 5.34 (s, 2H), 4.23 (s, 1H), 3.09-2.93 (m, 4H), 2.27- 2.18 (m, 3H), 1.27-1.13 (m, 6H). |
| 10 | 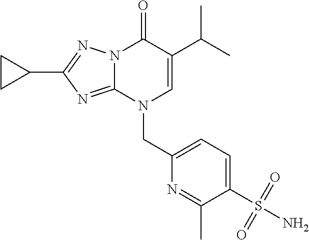 [SMILES: Cc1nc(Cn2cc(C(C)C)c(=O)n3nc(C4CC4)nc23)ccc1S(N)(=O)=O] | ESI-MS m/z = 403.15. [M + H]⁺; Calculated MW: 402.1. ¹H NMR (300 MHz, DMSO-d₆) δ 8.13 (d, J = 8.1 Hz, 1H), 7.99 (s, 1H), 7.61 (br, 2H), 7.31 (d, J = 8.2 Hz, 1H), 5.47 (s, 2H), 3.09-2.96 (m, 1H), 2.70 (s, 3H), 2.10-1.93 (m, 1H), 1.20 (d, J = 6.9 Hz, 6H), 1.01-0.90 (m, 2H), 0.90-0.77 (m, 2H). |
| 11 | 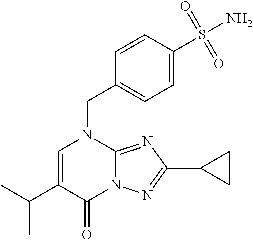 [SMILES: CC(C)c1cn(Cc2ccc(S(N)(=O)=O)cc2)c2nc(C3CC3)nn2c1=O] | ESI-MS m/z = 388.15 [M + H]⁺; Calculated: 387.1. ¹H NMR (400 MHz, DMSO-d₆) δ 8.02 (s, 1H), 7.84-7.76 (m, 2H), 7.56-7.50 (m, 2H), 7.34 (s, 2H), 5.39 (s, 2H), 3.02-2.95 (m, 1H), 2.07-2.01 (m, 1H), 1.19 (d, J = 6.9 Hz, 6H), 1.02-0.98 (m, 2H), 0.89- 0.86 (m, 2H). |
| 12 | 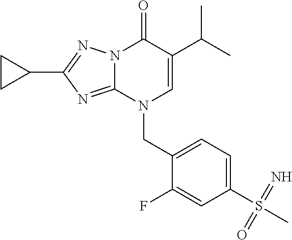 [SMILES: CC(C)c1cn(Cc2ccc(S(C)(=N)=O)cc2F)c2nc(C3CC3)nn2c1=O] | ESI-MS m/z = 404.20 [M + H]⁺; Calculated MW: 403.1. ¹H NMR (400 MHz, DMSO-d6) δ 7.99 (s, 1H), 7.84-7.67 (m, 2H), 7.51 (t, J = 7.6 Hz, 1H), 5.46 (s, 2H), 4.40 (s, 1H), 3.10 (s, 3H), 3.06-2.89 (m, 1H), 2.09-1.93 (m, 1H), 1.19 (d, J = 6.9 Hz, 6H), 1.03-0.92 (m, 2H), 0.91-0.80 (m, 2H). ¹⁹F NMR (377 MHz, DMSO) δ −114.96. |
| 13 | 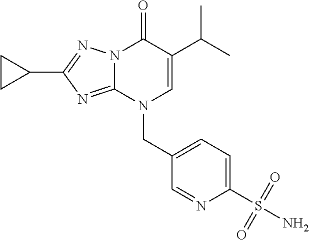 [SMILES: CC(C)c1cn(Cc2ccc(S(N)(=O)=O)nc2)c2nc(C3CC3)nn2c1=O] | ESI-MS m/z = 389.00 [M + H]⁺; Calculated: 388.1. ¹H NMR (400 MHz, DMSO-d₆) δ 8.79 (d, J = 2.2 Hz, 1H), 8.09-8.00 (m, 2H), 7.90 (d, J = 8.1 Hz, 1H), 7.44 (s, 2H), 5.43 (s, 2H), 2.98 (p, J = 6.9 Hz, 1H), 2.08-2.00 (m, 1H), 1.19 (d, J = 6.9 Hz, 6H), 1.00 (dt, J = 8.2, 3.2 Hz, 2H), 0.99-0.85 (m, 2H). |
| 14 | 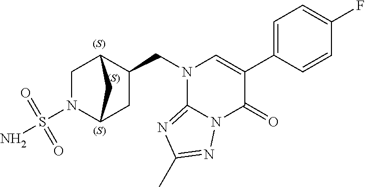 | ESI-MS m/z = 433.05 [M + H]⁺; Calculated: 432.1. ¹H NMR (400 MHz, DMSO-d₆) δ 8.43 (s, 1H), 7.76-7.67 (m, 2H), 7.35-7.25 (m, 2H), 6.67 (s, 2H), 4.22-4.11 (m, 1H), 4.05-3.94 (m, 2H), 3.13-3.04 (m, 1H), 2.74 (d, J = 9.0 Hz, 1H), 2.48-2.38 (m, 5H), 1.91 (t, J = 10.6 Hz, 1H), 1.74-1.54 (m, 2H), 1.35-1.25 (m, 1H). ¹⁹F NMR (376 MHz, DMSO-d₆) δ −114.64. |
| 15 | 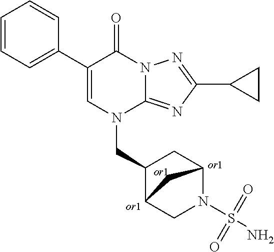 | ESI-MS m/z = 441.15 [M + H]+; Calculated: 440.2. ¹H NMR (400 MHz, DMSO-d₆) δ 8.40 (s, 1H), 7.73-7.60 (m, 2H), 7.45 (t, J = 7.6 Hz, 2H), 7.40- 7.29 (m, 1H), 6.67 (s, 2H), 4.13 (dd, J = 13.8, 9.3 Hz, 1H), 4.04- 3.90 (m, 2H), 3.08 (dd, J = 9.1, 3.6 Hz, 1H), 2.73 (d, J = 9.1 Hz, 1H), 2.46-2.33 (m, 2H), 2.16- 2.08 (m, 1H), 1.90 (dd, J = 12.5, 9.0 Hz, 1H), 1.68 (d, J = 10.3 Hz, 1H), 1.61 (d, J = 10.4 Hz, 1H), 1.35-1.25 (m, 1H), 1.09-1.02 (m, 2H), 0.98-0.92 (m, 2H). |
| 16 | 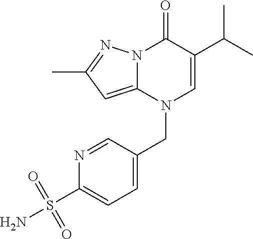 [SMILES: Cc1cc2n(Cc3ccc(S(N)(=O)=O)nc3)cc(C(C)C)c(=O)n2n1] | ESI-MS m/z = 362.10. [M + H]⁺; Calculated: 361.1. ¹H NMR (400 MHz, DMSO-d₆) δ 8.76 (d, J = 2.1 Hz, 1H), 8.01 (s, 1H), 7.98-7.87 (m, 2H), 7.47 (s, 2H), 6.10 (s, 1H), 5.39 (s, 2H), 3.01 (h, J = 6.8 Hz, 1H), 2.24 (s, 3H), 1.21 (d, J = 6.9 Hz, 6H). |
| 17 | 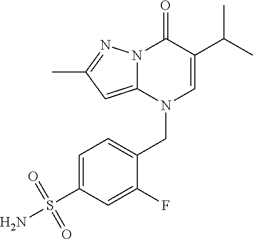 [SMILES: Cc1cc2n(Cc3ccc(S(N)(=O)=O)cc3F)cc(C(C)C)c(=O)n2n1] | ESI-MS m/z = 379.15. [M + H]⁺; Calculated: 378.1. ¹H NMR (400 MHz, DMSO-d₆) δ 7.93 (s, 1H), 7.71-7.61 (m, 2H), 7.60-7.43 (m, 3H), 6.02 (s, 1H), 5.43 (s, 2H), 3.08-2.98 (m, 1H), 2.25 (s, 3H), 1.20 (d, J = 6.9 Hz, 6H). ¹⁹F NMR (376 MHz, DMSO-d₆) δ −114.502. |
| 18 | 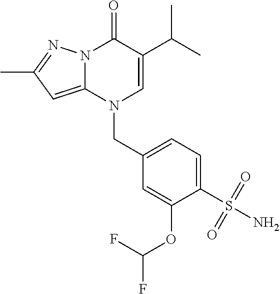 [SMILES: Cc1cc2n(Cc3ccc(S(N)(=O)=O)c(OC(F)F)c3)cc(C(C)C)c(=O)n2n1] | ESI-MS m/z = 427.10. [M + H]⁺; Calculated MW: 426.1. ¹H NMR (400 MHz, DMSO-d₆) δ 7.98 (d, J = 1.4 Hz, 1H), 7.86- 7.80 (m, 1H), 7.45 (br, 2H), 7.40- 7.00 (m, 3H), 6.02 (s, 1H), 5.34 (s, 2H), 3.09-2.95 (m, 1H), 2.24 (s, 3H), 1.20 (d, J = 6.9 Hz, 6H). ¹⁹F NMR (282 MHz, DMSO) δ −82.274. |
| 19 | 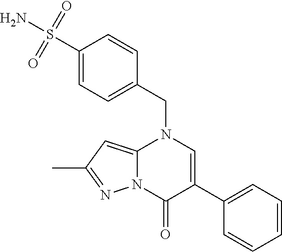 [SMILES: Cc1cc2n(Cc3ccc(S(N)(=O)=O)cc3)cc(-c3ccccc3)c(=O)n2n1] | ESI-MS m/z = 395.05. [M + H]⁺; Calculated: 394.1. ¹H NMR (400 MHz, DMSO-d₆) δ 8.36 (s, 1H), 7.83-7.70 (m, 2H), 7.61 (d, J = 7.6 Hz, 2H), 7.52 (d, J = 8.1 Hz, 2H), 7.37-7.27 (m, 4H), 7.26-7.20 (m, 1H), 6.02 (s, 1H), 5.34 (s, 2H), 2.18 (s, 3H). |
| 20 | 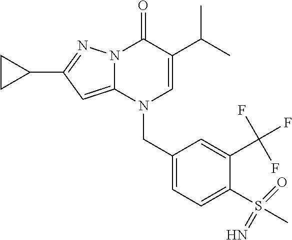 [SMILES: CC(C)c1cn(Cc2ccc(S(C)(=N)=O)c(C(F)(F)F)c2)c2cc(C3CC3)nn2c1=O] | ESI-MS m/z = 453.10 [M + H]⁺; Calculated:452.1. ¹H NMR (400 MHz, DMSO-d₆) δ 8.24 (d, J = 8.2 Hz, 1H), 8.10- 7.90 (m, 2H), 7.89-7.64 (m, 1H), 6.09 (s, 1H), 5.39 (s, 2H), 4.57 (s, 1H), 3.10 (s, 3H), 3.06-2.94 (m, 1H), 2.03-1.86 (m, 1H), 1.19 (d, J = 6.9 Hz, 3H), 1.19 (d, J = 6.9 Hz, 3H), 1.02-0.85 (m, 2H), 0.80- 0.66 (m, 2H). |
| 21 | 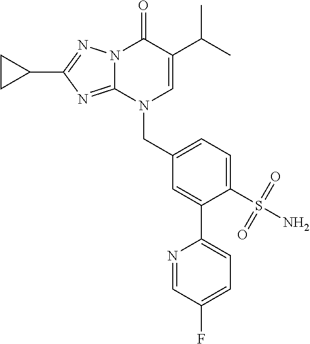 [SMILES: CC(C)c1cn(Cc2ccc(S(N)(=O)=O)c(-c3ccc(F)cn3)c2)c2nc(C3CC3)nn2c1=O] | ESI-MS m/z = 483.10 [M + H]⁺; Calculated: 482.2. ¹H NMR (400 MHz, DMSO-d₆) δ 8.61 (s, 1H), 8.46 (d, J = 4.8 Hz, 1H), 8.06-7.96 (m, 2H), 7.62 (dd, J = 8.2, 1.8 Hz, 1H), 7.48 (d, J = 16.0 Hz, 3H), 7.38 (dd, J = 6.4, 4.8 Hz, 1H), 5.40 (s, 2H), 3.02-2.96 (m, 1H), 2.06-2.00 (m, 1H), 1.17 (d, J = 6.9 Hz, 6H), 1.02-0.95 (m, 2H), 0.90-0.80 (m, 2H). |
| 22 | 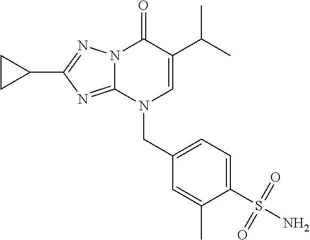 [SMILES: Cc1cc(Cn2cc(C(C)C)c(=O)n3nc(C4CC4)nc23)ccc1S(N)(=O)=O] | ESI-MS m/z = 402.15 [M + H]⁺, Calculated: 401.2 ¹H NMR (300 MHz, DMSO-d₆) δ 8.04-7.98 (m, 1H), 7.82 (d, J = 8.1 Hz, 1H), 7.38 (d, J = 2.6 Hz, 3H), 7.30 (dd, J = 8.1, 1.9 Hz, 1H), 5.35 (br, 2H), 2.99 (m,1H), 2.57 (s, 3H), 2.05 (m, 1H), 1.19 (d, J = 6.9 Hz, 6H), 1.07-0.82 (m, 4H). |
| 23 | 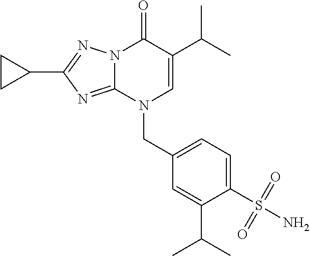 [SMILES: CC(C)c1cc(Cn2cc(C(C)C)c(=O)n3nc(C4CC4)nc23)ccc1S(N)(=O)=O] | ESI-MS m/z = 430.15 [M + H]⁺; Calculated:429.2. ¹H NMR (400 MHz, DMSO-d6) δ 8.07 (s, 1H), 7.78 (d, J = 8.2 Hz, 1H), 7.65 (d, J = 1.8 Hz, 1H), 7.47 (s, 2H), 7.27 (dd, J = 8.2, 1.8 Hz, 1H), 5.34 (s, 2H), 3.83 (p, J = 6.7 Hz, 1H), 3.02-2.98 (m, 1H), 2.05 (tt, J = 8.3, 4.8 Hz, 1H), 1.19 (dd, J = 6.9, 5.3 Hz, 12H), 1.04-0.87 (m, 4H). |
| 24 | 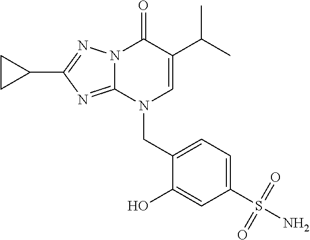 [SMILES: CC(C)c1cn(Cc2ccc(S(N)(=O)=O)cc2O)c2nc(C3CC3)nn2c1=O] | ESI-MS m/z = 404.0[M + H]⁺; Calculated: 403.1. ¹H NMR (300 MHz, DMSO-d₆) δ 10.67 (s, 1H), 7.91 (s, 1H), 7.36- 7.10 (m, 5H), 5.26 (s, 2H), 2.98 (p, J = 6.9 Hz, 1H), 2.08-2.00 (m, 1H), 1.17 (d, J = 6.9 Hz, 6H), 1.05- 0.94 (m, 2H), 0.88 (q, J = 3.6 Hz, 2H). |
| 25 | 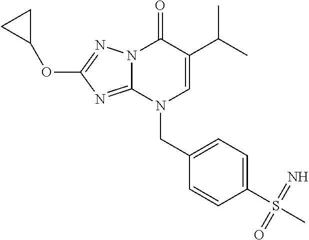 [SMILES: CC(C)c1cn(Cc2ccc(S(C)(=N)=O)cc2)c2nc(OC3CC3)nn2c1=O] | ESI-MS m/z = 402.15 [M + H]⁺; Calculated: 401.2. ¹H NMR (400 MHz, DMSO-d₆) δ 8.03 (s, 1H), 7.93-7.88 (m, 2H), 7.61-7.54 (m, 2H), 5.38 (s, 2H), 4.29-4.23 (m, 1H), 4.23-4.19 (m, 1H), 3.06-3.02 (m, 3H), 3.02- 2.93 (m, 1H), 1.24-1.15 (m, 6H), 0.81-0.71 (m, 4H). |
| 26 | 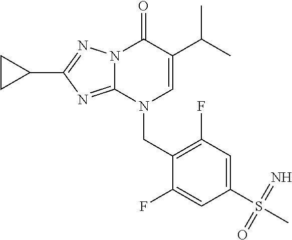 [SMILES: CC(C)c1cn(Cc2c(F)cc(S(C)(=N)=O)cc2F)c2nc(C3CC3)nn2c1=O] | ESI-MS m/z = 422.05 [M + H]⁺; Calculated: 421.0. ¹H NMR (400 MHz, DMSO-d₆) δ 8.04 (s, 1H), 7.65 (d, J = 7.1 Hz, 2H), 5.48 (s, 2H), 4.55 (s, 1H), 3.14 (s, 3H), 3.07-2.91 (m, 1H), 2.05-1.94 (m, 1H), 1.20 (d, J = 6.9 Hz, 6H), 1.02-0.93 (m, 2H), 0.89-0.77 (m, 2H). ¹⁹F NMR (376 MHz, DMSO-d₆) δ −109.59. |
| 27 | 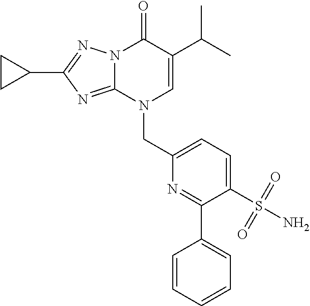 [SMILES: CC(C)c1cn(Cc2ccc(S(N)(=O)=O)c(-c3ccccc3)n2)c2nc(C3CC3)nn2c1=O] | ESI-MS m/z = 465.20 [M + H]⁺; Calculated:464.2. ¹H NMR (400 MHz, DMSO-d6) δ 8.40 (d, J = 8.3 Hz, 1H), 8.03 (s, 1H), 7.68-7.48 (m, 5H), 7.47- 7.33 (m, 3H), 5.57 (s, 2H), 3.08- 2.93 (m, 1H), 2.05-1.96 (m, 1H), 1.17 (d, J = 6.9 Hz, 6H), 1.03-0.92 (m, 2H), 0.91-0.81 (m, 2H). |
| 28 | 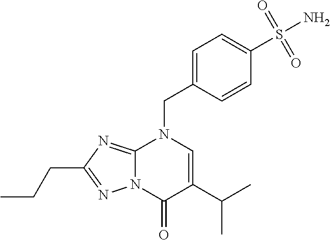 [SMILES: CCCc1nc2n(Cc3ccc(S(N)(=O)=O)cc3)cc(C(C)C)c(=O)n2n1] | ESI-MS m/z = 390.15 [M + H]⁺; Calculated: 389.2 ¹H NMR (300 MHz, DMSO-d₆) δ 8.05 (s, 1H), 7.79 (d, J = 8.2 Hz, 2H), 7.53 (d, J = 8.2 Hz, 2H), 7.35 (s, 2H), 5.44 (s, 2H), 3.06-2.94 (m, 1H), 2.65 (t, J = 7.4 Hz, 2H), 1.78-1.62 (m, 2H), 1.20 (d, J = 6.9 Hz, 6H), 0.91 (t, J = 7.4 Hz, 3H). |
| 29 | 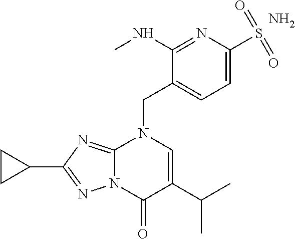 | ESI-MS m/z = 418.15 [M + H]⁺, as sulfonic acid; Calculated: 417.1. ¹H NMR (400 MHz, DMSO-d₆) δ 7.93 (s, 1H), 7.27 (d, J = 7.5 Hz, 1H), 7.17 (s, 2H), 6.98 (d, J = 7.5 Hz, 1H), 6.88 (q, J = 4.5 Hz, 1H), 5.16 (s, 2H), 3.06-2.90 (m, 4H), 2.15-1.92 (m, 1H), 1.17 (d, J = 6.9 Hz, 6H), 1.06-0.96 (m, 2H), 0.92-0.80 (m, 2H). |
| 30 | 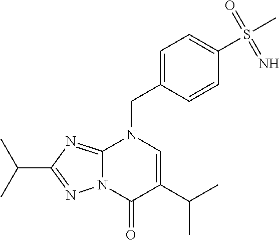 [SMILES: CC(C)c1nc2n(Cc3ccc(S(C)(=N)=O)cc3)cc(C(C)C)c(=O)n2n1] | ESI-MS m/z = 388.25 [M + H]⁺; Calculated: 387.2. ¹H NMR (400 MHz, DMSO-d6) δ 8.07 (s, 1H), 7.91 (d, J = 8.2 Hz, 2H), 7.60 (d, J = 8.1 Hz, 2H), 5.46 (s, 2H), 4.21 (s, 1H), 3.04 (s, 3H), 3.03-2.95 (m, 2H), 1.27 (d, J = 6.9 Hz, 6H), 1.19 (d, J = 6.9 Hz, 6H). |
| 31 Rac | 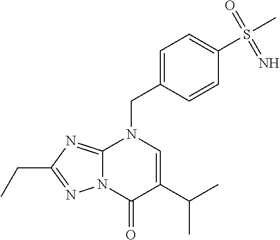 [SMILES: CCc1nc2n(Cc3ccc(S(C)(=N)=O)cc3)cc(C(C)C)c(=O)n2n1] | ESI-MS m/z = 374.05 [M + H]⁺; Calculated:373.2. ¹H NMR (400 MHz, DMSO-d₆) δ 8.09 (s, 1H), 7.96-7.89 (m, 2H), 7.62-7.55 (m, 2H), 5.48 (s, 2H), 4.23 (s, 1H), 3.06 (s, 3H), 3.05- 2.96 (m, 1H), 2.76-2.66 (m, 2H), 1.29-1.24 (m, 3H), 1.22-1.19 (m, 6H). |
| 31A | 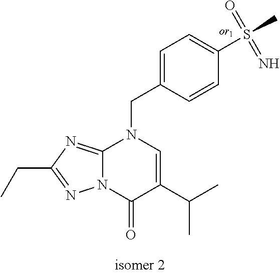 [SMILES: CCc1nc2n(Cc3ccc([S@@](C)(=N)=O)cc3)cc(C(C)C)c(=O)n2n1] | ESI-MS m/z = 374.05 [M + H]⁺; Calculated:373.2. ¹H NMR (400 MHz, DMSO-d₆) δ 8.07 (s, 1H), 7.94-7.88 (m, 2H), 7.60-7.53 (m, 2H), 5.47 (s, 2H), 4.21 (s, 1H), 3.07-3.03 (m, 3H). 3.03-2.93 (m, 1H), 2.75-2.65 (m, 2H), 1.28-1.20 (m, 3H), 1.23- 1.17 (m, 6H). |
| 31B | 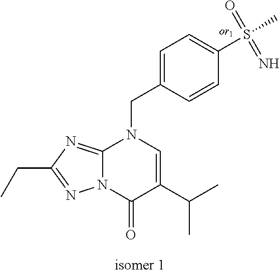 [SMILES: CCc1nc2n(Cc3ccc([S@](C)(=N)=O)cc3)cc(C(C)C)c(=O)n2n1] | ESI-MS m/z = 374.10 [M + H]⁺; Calculated:373.2. ¹H NMR (400 MHz, DMSO-d₆) δ 8.07 (s, 1H), 7.95-7.88 (m, 2H), 7.61-7.54 (m, 2H), 5.47 (s, 2H), 4.22 (s, 1H), 3.05 (s, 3H), 3.04- 2.95 (m, 1H), 2.75-2.65 (m, 2H), 1.27-1.16 (m, 9H). |
| 32 Rac | 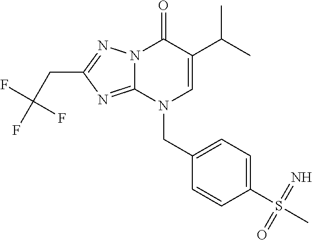 [SMILES: CC(C)c1cn(Cc2ccc(S(C)(=N)=O)cc2)c2nc(CC(F)(F)F)nn2c1=O] | ESI-MS m/z = 428.15 [M + H]⁺; Calculated: 427.1. ¹H NMR (400 MHz, DMSO-d₆) δ 8.15 (s, 1H), 7.92 (d, J = 8.3 Hz, 2H), 7.60 (d, J = 8.4 Hz, 2H), 5.51 (s, 2H), 4.24 (s, 1H), 4.00-3.87 (m, 2H), 3.06 (s, 3H), 3.05-2.98 (m, 1H), 1.22 (d, J = 6.9 Hz, 6H). ¹⁹F NMR (377 MHz, DMSO-d₆) δ −62.73. |
| 32A | 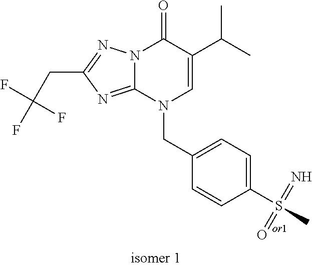 [SMILES: CC(C)c1cn(Cc2ccc([S@](C)(=N)=O)cc2)c2nc(CC(F)(F)F)nn2c1=O] | ESI-MS m/z = 428.00 [M + H]⁺; Calculated: 427.1. ¹H NMR (400 MHz, DMSO-d₆) δ 8.13 (d, J = 0.8 Hz, 1H), 7.95- 7.85 (m, 2H), 7.62-7.51 (m, 2H), 5.48 (s, 2H), 4.22 (s, 1H), 4.02- 3.82 (m, 2H), 3.07-2.97 (m, 4H), 1.21 (d, J = 7.0 Hz, 6H). ¹⁹F NMR (377 MHz, DMSO-d₆) δ −62.70. |
| 32B | 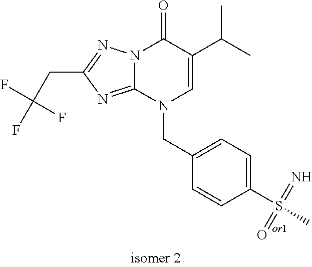 [SMILES: CC(C)c1cn(Cc2ccc([S@@](C)(=N)=O)cc2)c2nc(CC(F)(F)F)nn2c1=O] | ESI-MS m/z = 428.05 [M + H]⁺; Calculated: 427.1. ¹H NMR (400 MHz, DMSO-d₆) δ 8.15-8.10 (m, 1H), 7.93-7.87 (m, 2H), 7.63-7.50 (m, 2H), 5.48 (s, 2H), 4.22 (s, 1H), 4.03-3.78 (m, 2H), 3.12-2.94 (m, 4H), 1.21 (d, J = 6.9 Hz, 6H). ¹⁹F NMR (377 MHz, DMSO-d6) δ −62.70. |
| 33 | 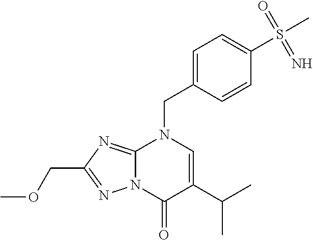 | ESI-MS m/z = 390.10 [M + H]⁺; Calculated: 389.2. ¹H NMR (400 MHz, DMSO-d₆) δ 8.15-8.05 (br, 1H), 7.94-7.86 (m, 2H), 7.59-7.52 (m, 2H), 5.49 (s, 2H), 4.46 (s, 2H), 4.25 (s, 1H), 3.31 (s, 3H), 3.07-2.96 (m, 4H), 1.24-1.16 (m, 6H). |
| 34 | 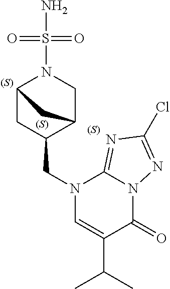 | ESI-MS m/z = 401.05 [M + H]⁺; Calculated: 400.1. ¹H NMR (400 MHz, DMSO-d₆) δ 7.99 (s, 1H), 6.66 ( s, 2H), 4.13- 4.02 (m, 1H), 3.99-3.88 (m, 2H), 3.11-3.05 (m, 1H), 3.04-2.94 (m, 1H), 2.73 (d, J = 9.0 Hz, 1H), 2.37-2.26 (m, 2H), 1.94-1.85 (m, 1H), 1.71-1.56 (m, 2H), 1.30- 1.23 (m, 1H), 1.20 (d, J = 6.6 Hz, 6H). |
| 35 | 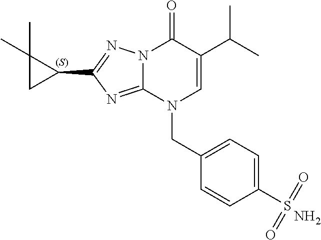 | ESI-MS m/z = 416.15. [M + H]⁺; Calculated MW: 415.2. ¹H NMR (400 MHz, DMSO-d₆) δ 8.04 (s, 1H), 7.80-7.73 (m, 2H), 7.56-7.48 (m, 2H), 7.35 (br, 2H), 5.53-5.32 (m, 2H), 3.07-2.89 (m, 1H), 1.94-1.85 (m, 1H), 1.24- 1.13 (m, 9H), 1.13-1.04 (m, 1H), 1.00 (s, 3H), 0.97-0.89 (m, 1H). |
| 36 | 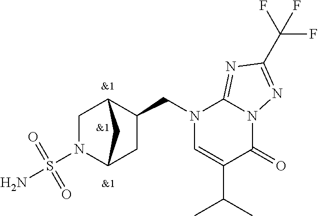 [SMILES: CC(C)c1cn(C[C@H]2C[C@H]3C[C@@H]2CN3S(N)(=O)=O)c2nc(C(F)(F)F)nn2c1=O] | ESI-MS m/z = 435.13 [M + H]⁺; Calculated: 434.1. ¹H NMR (400 MHz, DMSO-d₆) δ 8.10 (s, 1H), 6.67 (s, 2H), 4.07 (m, 2H), 3.94 (s, 1H), 3.12-2.97 (m, 2H), 2.71 (d, J = 9.0 Hz, 1H), 2.34 (dd, J = 11.6, 4.5 Hz, 2H), 1.89 (m, 1H), 1.72-1.57 (m, 2H), 1.32- 1.17 (m, 7H). |
| 37 | 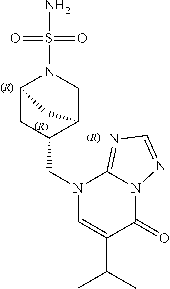 | ESI-MS m/z = 367.10 [M + H]⁺; Calculated: 366.1. ¹H NMR (400 MHz, DMSO-d₆) δ 8.24 (s, 1H), 8.01 (s, 1H), 6.67 (s, 2H), 4.14 (dd, J = 13.8, 9.0 Hz, 1H), 4.02-3.91 (m, 2H), 3.07 (dd, J = 9.1, 3.5 Hz, 1H), 3.05-2.95 (m, 1H), 2.71 (d, J = 9.1 Hz, 1H), 2.41-2.29 (m, 2H), 1.93-1.84 (m, 1H), 1.68 (d, J = 10.2 Hz, 1H), 1.61 (d, J = 10.4 Hz, 1H), 1.32- 1.25 (m, 1H), 1.22 (s, 3H), 1.21 (s, 3H). |
| 38 | 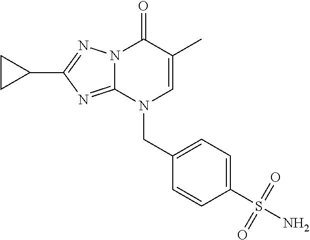 [SMILES: Cc1cn(Cc2ccc(S(N)(=O)=O)cc2)c2nc(C3CC3)nn2c1=O] | ESI-MS m/z = 360.15 [M + H]⁺; Calculated: 359.1. ¹H NMR (300 MHz, DMSO-d₆) δ 8.09 (d, J = 1.2 Hz, 1H), 7.84- 7.76 (m, 2H), 7.58-7.50 (m, 2H), 7.37 (s, 2H), 5.35 (s, 2H), 2.12- 1.96 (m, 4H), 1.04-1.00 (m, 2H), 0.97-0.85 (m, 2H). |
| 39 | 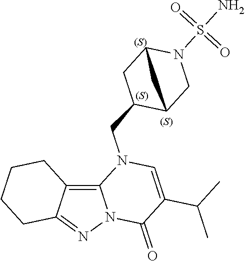 | ESI-MS m/z = 420.15 [M + H]+; Calculated:419.2 1H NMR (400 MHz, DMSO-d6) δ 7.69 (s, 1H), 6.67 (s, 2H), 4.08- 3.92 (m, 3H), 3.09 (dd, J = 9.2, 3.5 Hz, 1H), 2.99 (p, J = 7.0 Hz, 1H), 2.83-2.73 (m, 3H), 2.67 (s, 2H), 2.30 (s, 1H), 2.09 (q, J = 7.9, 7.3 Hz, 1H), 1.91 (t, J = 10.5 Hz, 1H), 1.78-1.74 (m, 4H), 1.69 (d, J = 10.3 Hz, 1H), 1.62 (d, J = 10.3 Hz, 1H), 1.27 (dt, J = 17.2, 5.4 Hz, 1H), 1.17 (dd, J = 6.9, 2.9 Hz, 6H). |
| 40 | 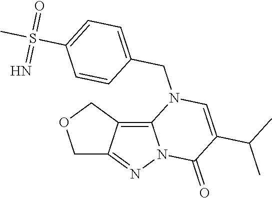 [SMILES: CC(C)c1cn(Cc2ccc(S(C)(=N)=O)cc2)c2c3c(nn2c1=O)COC3] | ESI-MS m/z = 387.10 [M + H]+; Calculated: 386.1. 1H NMR (400 MHz, DMSO-d6) δ 7.99 (s, 1H), 7.92 (d, J = 8.1 Hz, 2H), 7.41 (d, J = 8.1 Hz, 2H), 5.38 (s, 2H), 4.75 (s, 2H), 4.56-4.45 (m, 2H), 4.25 (s, 1H), 3.10-2.98 (m, 4H), 1.23 (d, J = 6.9 Hz, 6H). |
| 41 | 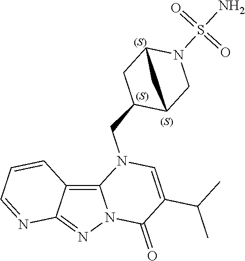 | ESI-MS m/z = 417.15 [M + H]+; Calculated: 416.2. 1H NMR (400 MHz, DMSO-d6) δ 8.79 (d, J = 4.1 Hz, 1H), 8.42 (d, J = 8.5 Hz, 1H), 8.15 (s, 1H), 7.17 (dd, J = 8.6, 4.2 Hz, 1H), 6.63 (s, 2H), 4.56-4.34 (m, 2H), 3.98 (s, 1H), 3.21-3.00 (m, 2H), 2.67 (d, J = 9.2 Hz, 1H), 2.41 (s, 1H), 2.29 (d, J = 9.0 Hz, 1H), 1.98 (t, J = 10.3 Hz, 1H), 1.81 (d, J = 10.2 Hz, 1H), 1.64 (d, J = 10.3 Hz, 1H), 1.41 (d, J = 11.3 Hz, 1H), 1.35-1.21 (m, 6H). |
| 42 | 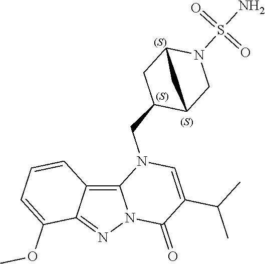 | ESI-MS m/z = 446.30 [M + H]+; Calculated: 445.2. 1H NMR (400 MHz, DMSO-d6) δ 8.08 (s, 1H), 7.47 (d, J = 8.6 Hz, 1H), 7.04 (dd, J = 8.7, 7.4 Hz, 1H), 6.84 (d, J = 7.4 Hz, 1H), 6.63 (s, 2H), 4.47-4.33 (m, 2H), 3.97 (d, J = 7.3 Hz, 4H), 3.20-3.11 (m, 1H), 3.07 (dd, J = 9.2, 3.6 Hz, 1H), 2.63 (d, J = 9.2 Hz, 1H), 2.40 (s, 1H), 2.29 (s, 1H), 1.95 (t, J = 10.8 Hz, 1H), 1.79 (t, J = 9.5 Hz, 1H), 1.68-1.61 (m, 1H), 1.41 (d, J = 11.5 Hz, 1H), 1.27 (dd, J = 6.9, 2.4 Hz, 6H). |
| 43 | 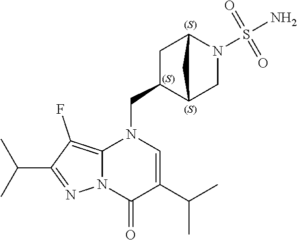 | ESI-MS m/z = 426.20 [M + H]+; Calculated:425.2. 1H NMR (400 MHz, DMSO-d6) δ 7.79 (s, 1H), 6.69 (s, 2H), 4.13- 3.79 (m, 3H), 3.20-3.06 (m, 2H), 3.06-2.91 (m, 1H), 2.76 (d, J = 9.1 Hz, 1H), 2.34-2.29 (m, 1H), 2.24-2.16 (m, 1H), 2.09 (s, 1H), 1.95-1.85 (m, 1H), 1.71-1.60 (m, 2H), 1.30 (d, J = 7.0 Hz, 6H), 1.18 (d, J = 6.9 Hz, 6H). 19F NMR (377 MHz, DMSO-d6) δ −185.83 |
| 44 | 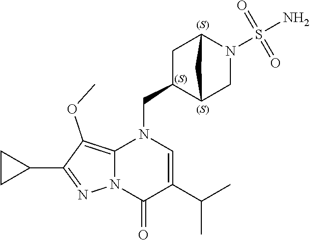 | ESI-MS m/z = 436.15 [M + H]+; Calculated:435.2. 1H NMR (400 MHz, DMSO-d6) δ 7.70 (s, 1H), 6.68 (s, 2H), 4.07- 4.01 (m, 1H), 3.98-3.89 (m, 2H), 3.86 (s, 3H), 3.10 (dd, J = 9.2, 3.5 Hz, 1H), 3.02-2.91 (m, 1H), 2.73 (d, J = 9.1 Hz, 1H), 2.33-2.27 (m, 1H), 2.26-2.16 (m, 1H), 2.07- 1.97 (m, 1H), 1.90 (t, J = 10.6 Hz, 1H), 1.71-1.59 (m, 2H), 1.31-1.23 (m, 1H), 1.16 (d, J = 6.9 Hz, 6H), 1.05-0.87 (m, 4H). |
| 45 | | ESI-MS m/z = 431.05 [M + H]+; |
| | | Calculated: 430.2. |
| | | 1H NMR (400 MHz, DMSO-d6) δ |
| | | 7.87 (s, 1H), 6.67 (s, 2H), 4.20- |
| | | 4.03 (m, 2H), 3.96 (s, 1H), 3.15- |
| | | 3.04 (m, 1H), 3.04-2.91 (m, 1H), |
| | | 2.77 (d, J = 9.0 Hz, 1H), 2.37- |
| | | 2.25 (m, 2H), 2.11-2.00 (m, 1H), |
| | | 2.00-1.90 (m, 1H), 1.77-1.68 |
| | | (m, 1H), 1.64 (d, J = 10.4 Hz, 1H), |
| | | 1.33-1.24 (m, 1H), 1.23-1.13 |
| | | (m, 6H), 1.14-1.05 (m, 2H), 1.05- |
| | | 0.96 (m, 2H). |
| 46 | | ESI-MS m/z = 448.10 [M + H]+; |
| | | Calculated: 447.2. |
| | | 1H NMR (400 MHz, DMSO-d6) δ |
| | | 7.89 (s, 1H), 6.54 (s, 1H), 4.07- |
| | | 3.95 (m, 2H), 3.95-3.84 (m, 1H), |
| | | 3.51-3.41 (m, 1H), 3.20-3.06 |
| | | (m, 1H), 3.05-2.93 (m, 1H), 2.84- |
| | | 2.67 (m, 4H), 2.30 (q, J = 4.2 |
| | | Hz, 2H), 1.90-1.72 (m, 2H), 1.52 |
| | | (d, J = 10.3 Hz, 1H), 1.32-1.23 |
| | | (m, 1H), 1.20 (d, J = 6.9 Hz, 6H). |
| | | 19F NMR (377 MHz, DMSO) δ |
| | | −57.62. |
| 47 | 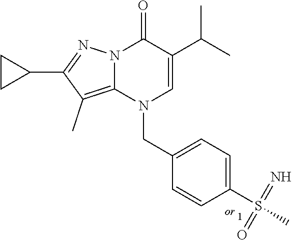 [SMILES: Cc1c(C2CC2)nn2c(=O)c(C(C)C)cn(Cc3ccc([S@@](C)(=N)=O)cc3)c12] | ESI-MS m/z = 399.10 [M + H]+; Calculated MW: 398.18. 1H NMR (400 MHz, DMSO-d6) δ 7.96-7.88 (m, 2H), 7.79 (br, 1H), 7.30 (m, 2H), 5.58 (s, 2H), 4.22 (s, 1H), 3.04 (d, J = 1.1 Hz, 3H), 3.01-2.97 (m, 1H), 2.00 (s, 3H), 1.83-1.86 (m, 1H), 1.18 (d, J = 6.9 Hz, 6H), 0.90-0.88 (m, 2H), 0.86-0.82 (m, 2H). |
| 48 | 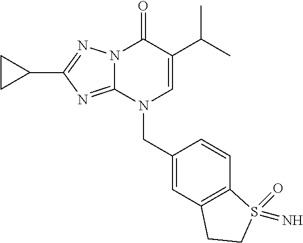 [SMILES: CC(C)c1cn(Cc2ccc3c(c2)CCS3(=N)=O)c2nc(C3CC3)nn2c1=O] | ESI-MS m/z = 398.10 [M + H]+; Calculated:397.2. 1H NMR (400 MHz, DMSO-d6) δ 8.02-8.00 (m, 1H), 7.62 (d, J = 8.0 Hz, 1H), 7.47-7.40 (m, 2H), 5.37 (s, 2H), 4.51 (s, 1H), 3.44 (t, J = 6.8 Hz, 2H), 3.24 (td, J = 6.8, 2.5 Hz, 2H), 3.03-2.95 (m, 1H), 2.04 (tt, J = 8.3, 4.9 Hz, 1H), 1.19 (d, J = 6.9 Hz, 6H), 1.04-0.97 (m, 2H), 0.90 (dt, J = 4.7, 3.0 Hz, 2H). |
| 49 | 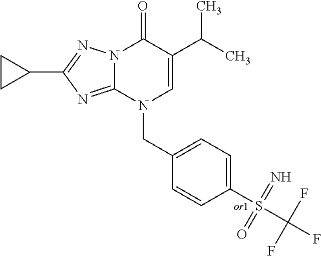 [SMILES: CC(C)c1cn(Cc2ccc(S(=N)(=O)C(F)(F)F)cc2)c2nc(C3CC3)nn2c1=O] | ESI-MS m/z = 440.15 [M + H]+; Calculated: 439.1. 1H NMR (400 MHz, DMSO-d6) δ 8.11-7.97 (m, 3H), 7.68 (d, J = 8.2 Hz, 2H), 6.87 (s, 1H), 5.49 (s, 2H), 3.07-2.92 (m, 1H), 2.06- 1.95 (m, 1H), 1.19 (d, J = 6.9 Hz, 6H), 1.03-0.94 (m, 2H), 0.92- 0.80 (m, 2H). |
| 50 | 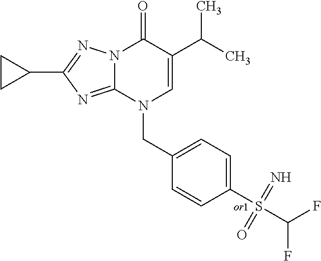 [SMILES: CC(C)c1cn(Cc2ccc(S(=N)(=O)C(F)F)cc2)c2nc(C3CC3)nn2c1=O] | ESI-MS m/z = 422.15 [M + H]+; Calculated: 421.5. 1H NMR (400 MHz, DMSO-d6) δ 8.10-7.85 (m, 3H), 7.63 (d, J = 8.1 Hz, 2H), 6.88 (t, J = 53.6 Hz, 1H), 5.62 (s, 1H), 5.46 (s, 2H), 3.08-2.91 (m, 1H), 2.10-1.97 (m, 1H), 1.19 (d, J = 6.9 Hz, 6H), 1.04-0.96 (m, 2H), 0.91-0.80 (m, 2H). |
| 51 | 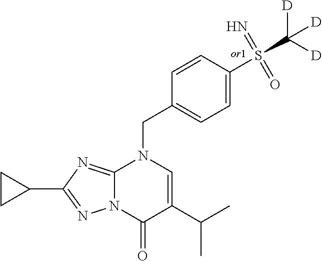 [SMILES: [2H]C([2H])([2H])[S@@](=N)(=O)c1ccc(Cn2cc(C(C)C)c(=O)n3nc(C4CC4)nc23)cc1] | ESI-MS m/z = 389.15 [M + H]+; Calculated 388.2. 1H NMR (400 MHz, DMSO-d6) δ 8.04 (s, 1H), 7.95-7.85 (m, 2H), 7.61-7.50 (m, 2H), 5.41 (s, 2H), 4.19 (s, 1H), 3.06-2.91 (m, 1H), 2.09-1.98 (m, 1H), 1.19 (d, J = 6.9 Hz, 6H), 1.04-0.96 (m, 2H), 0.93-0.86 (m, 2H). |
| 52 | 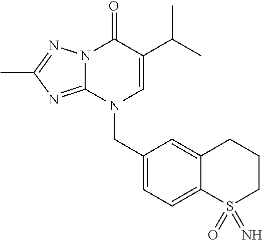 [SMILES: Cc1nc2n(Cc3ccc4c(c3)CCCS4(=N)=O)cc(C(C)C)c(=O)n2n1] | ESI-MS m/z = 386.10 [M + H]+; Calculated: 385.2. 1H NMR (400 MHz, DMSO-d6) δ 8.02 (s, 1H), 7.80 (d, J = 8.2 Hz, 1H), 7.33 (d, 1H), 7.25 (br, 1H), 5.36 (s, 2H), 4.43 (s, 1H), 3.39- 3.25 (m, 2H), 3.06-2.96 (m, 1H), 2.96-2.86 (m, 2H), 2.34 (s, 3H), 2.29-2.17 (m, 2H), 1.26-1.14 (m, 6H). |
| 53 | 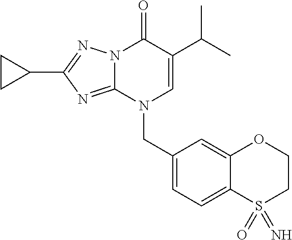 [SMILES: CC(C)c1cn(Cc2ccc3c(c2)OCCS3(=N)=O)c2nc(C3CC3)nn2c1=O] | ESI-MS m/z = 414.15 [M + H]+; Calculated:413.2. 1H NMR (400 MHz, DMSO-d6) δ 8.01 (s, 1H), 7.77 (d, J = 8.2 Hz, 1H), 7.12 (dd, J = 8.3, 1.7 Hz, 1H), 7.02(d, J = 1.6 Hz, 1H), 5.31 (s, 2H), 4.81 (s, 1H), 4.78-4.63 (m, 2H), 3.67-3.51 (m, 2H), 3.06- 2.91 (m, 1H), 2.11-2.00 (m, 1H), 1.19 (d, J = 7.0 Hz, 3H), 1.19 (d, J = 7.0 Hz, 3H), 1.06-0.97 (m, 2H), 1.00-0.86 (m, 2H). |
| 54 | 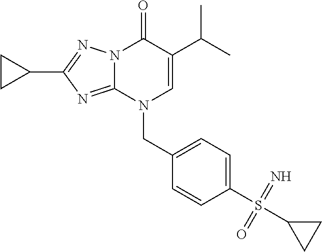 [SMILES: CC(C)c1cn(Cc2ccc(S(=N)(=O)C3CC3)cc2)c2nc(C3CC3)nn2c1=O] | ESI-MS m/z = 412.10 [M + H]⁺; Calculated: 411.2. ¹H NMR (400 MHz, DMSO-d₆) δ 8.05 (s, 1H), 7.92-7.83 (m, 2H), 7.60-7.52 (m, 2H), 5.49-5.36 (m, 2H), 4.19 (s, 1H), 3.06-2.93 (m, 1H), 2.70-2.58 (m, 1H), 2.10- 1.97 (m, 1H), 1.23-1.09 (m, 6H), 1.15-0.80 (m, 8H). |
| 55 | 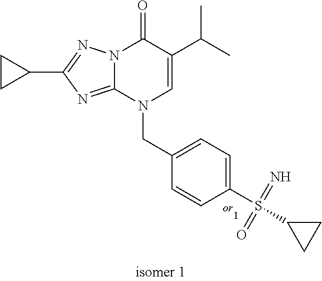 [SMILES: CC(C)c1cn(Cc2ccc([S@@](=N)(=O)C3CC3)cc2)c2nc(C3CC3)nn2c1=O] | ESI-MS m/z = 412.20 [M + H]⁺; Calculated: 411.2. ¹H NMR (400 MHz, DMSO-d₆) δ 8.03 (s, 1H), 7.87 (d, J = 8.2 Hz, 2H), 7.55 (d, J = 8.1 Hz, 2H), 5.41 (s, 2H), 4.20 (s, 1H), 3.04-2.92 (m, 1H), 2.68-2.57 (m, 1H), 2.11- 1.97 (m, 1H), 1.19 (d, J = 6.9 Hz, 6H), 1.13-1.04 (m, 1H), 1.03- 0.97 (m, 2H), 0.97-0.86 (m, 5H). |
| 56 Rac | 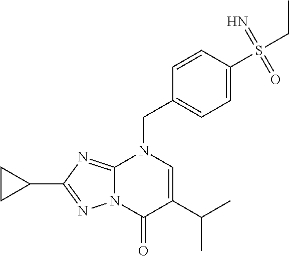 [SMILES: CCS(=N)(=O)c1ccc(Cn2cc(C(C)C)c(=O)n3nc(C4CC4)nc23)cc1] | ESI-MS m/z = 400.05 [M + H]+; Calculated:399.1. 1H NMR (400 MHz, DMSO-d6) δ 8.03 (s, 1H), 7.85 (d, J = 8.3 Hz, 2H), 7.56 (d, J = 8.3 Hz, 2H), 5.42 (s, 2H), 4.17 (s, 1H), 3.15-3.06 (m, 2H), 3.06-2.91 (m, 1H), 2.09- 1.98 (m, 1H), 1.18 (d, J = 6.9 Hz, 3H), 1.18 (d, J = 6.9 Hz, 3H), 1.07-0.93 (m, 5H), 0.92-0.84 (m, 2H). |
| 56A | 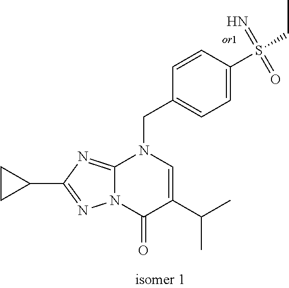 [SMILES: CC[S@](=N)(=O)c1ccc(Cn2cc(C(C)C)c(=O)n3nc(C4CC4)nc23)cc1] | ESI-MS m/z = 400.15 [M + H]+; Calculated:399.2. 1H NMR (400 MHz, DMSO-d6) δ 8.03 (s, 1H), 7.89-7.80 (m, 2H), 7.60-7.50 (m, 2H), 5.42 (s, 2H), 4.19 (s, 1H), 3.10 (q, J = 7.3 Hz, 2H), 3.03-2.90 (m, 1H), 2.11- 1.96 (m, 1H), 1.19 (d, J = 6.9 Hz, 6H), 1.07-0.96 (m, 5H), 0.91- 0.83 (m, 2H). |
| 57 | 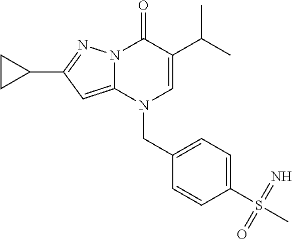 [SMILES: CC(C)c1cn(Cc2ccc(S(C)(=N)=O)cc2)c2cc(C3CC3)nn2c1=O] | ESI-MS m/z = 385.05 [M + H]+; Calculated:384.2. 1H NMR (400 MHz, DMSO-d6) δ 7.98-7.88 (m, 3H), 7.56-7.50 (m, 2H), 6.03 (s, 1H), 5.33 (s, 2H), 4.21 (s, 1H), 3.04 (s, 3H), 3.05- 2.94 (m, 1H), 1.99-1.88 (m, 1H), 1.23-1.17 (m, 6H), 0.98-0.87 (m, 2H), 0.78-0.70 (m, 2H). |
| 58 Rac | 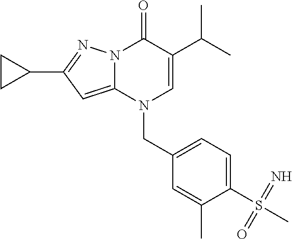 [SMILES: Cc1cc(Cn2cc(C(C)C)c(=O)n3nc(C4CC4)cc23)ccc1S(C)(=N)=O] | ESI-MS m/z = 399.10 [M + H]+; Calculated:398.2. 1H NMR (400 MHz, DMSO-d6) δ 7.97-7.89 (m, 2H), 7.37-7.33 (m, 1H), 7.33-7.27 (m, 1H), 6.02 (br, 1H), 5.25 (s, 2H), 4.27 (s, 1H), 3.08-3.03 (m, 3H), 3.03-2.96 (br, 1H), 2.65 (s, 3H), 1.97-1.89 (m, 1H), 1.24-1.16 (m, 6H), 0.97- 0.90 (m, 2H), 0.77-0.71 (m, 2H). |
| 58A | 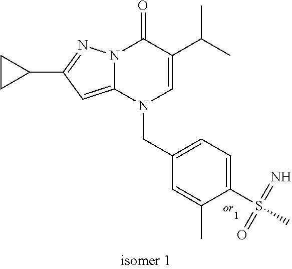 [SMILES: Cc1cc(Cn2cc(C(C)C)c(=O)n3nc(C4CC4)cc23)ccc1[S@@](C)(=N)=O] | ESI-MS m/z = 399.15 [M + H]+; Calculated: 398.2. 1H NMR (400 MHz, DMSO-d6) δ 7.97-7.90 (m, 2H), 7.38-7.26 (m, 2H), 6.03 (br, 1H), 5.25 (s, 2H), 4.27 (s, 1H), 3.07-2.96 (m, 4H), 2.65 (s, 3H), 1.97-1.89 (m, 1H), 1.25-1.14 (m, 6H), 0.97- 0.90 (m, 2H), 0.78-0.71 (m, 2H). |
| 59 Rac | 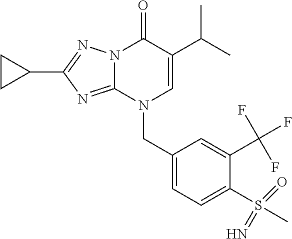 [SMILES: CC(C)c1cn(Cc2ccc(S(C)(=N)=O)c(C(F)(F)F)c2)c2nc(C3CC3)nn2c1=O] | ESI-MS m/z = 454.10 [M + H]+; Calculated:453.1. 1H NMR (400 MHz, DMSO-d6) δ 8.24 (d, J = 8.2 Hz, 1H), 8.11 (d, J = 6.5 Hz, 2H), 7.86 (d, J = 8.2 Hz, 1H), 5.47 (s, 2H), 4.64-4.52 (m, 1H), 3.11 (s, 3H), 3.00-2.96 (m, 1H), 2.04 (tt, J = 8.8, 4.9 Hz, 1H), 1.19 (d, J = 6.8 Hz, 6H), 1.04-0.98 (m, 2H), 0.92-0.86 (m, 2H). 19F NMR (377 MHz, DMSO) δ −55.05. |
| 59A | 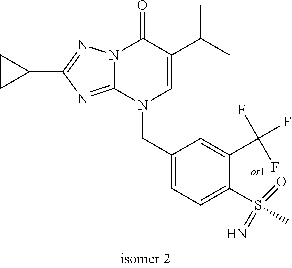 [SMILES: CC(C)c1cn(Cc2ccc([S@](C)(=N)=O)c(C(F)(F)F)c2)c2nc(C3CC3)nn2c1=O] | ESI-MS m/z = 454.05 [M + H]+; Calculated:453.1. 1H NMR (400 MHz, DMSO-d6) δ 8.24 (d, J = 8.2 Hz, 1H), 8.13- 8.08 (m, 2H), 7.85 (dd, J = 8.1, 1.8 Hz, 1H), 5.46 (s, 2H), 4.58 (d, J = 1.8 Hz, 1H), 3.10 (d, J = 1.5 Hz, 3H), 2.99 (p, J = 6.9 Hz, 1H), 2.04 (tt, J = 8.2, 4.8 Hz, 1H), 1.19 (d, J = 6.9 Hz, 6H), 1.01 (dq, J = 6.1, 3.6 Hz, 2H), 0.91-0.84 (m, 2H). 19F NMR (377 MHz, DMSO) δ −55.05. |
| 60 | 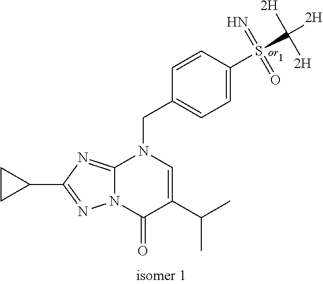 | ESI-MS m/z = 389.15 [M + H]+; Calculated: 388.2. 1H NMR (400 MHz, DMSO-d6) δ 8.04 (s, 1H), 7.95-7.85 (m, 2H), 7.61-7.50 (m, 2H), 5.41 (s, 2H), 4.19 (s, 1H), 3.06-2.91 (m, 1H), 2.09-1.98 (m, 1H), 1.19 (d, J = 6.9 Hz, 6H), 1.04-0.96 (m, 2H), 0.93-0.86 (m, 2H). |
| 61 | 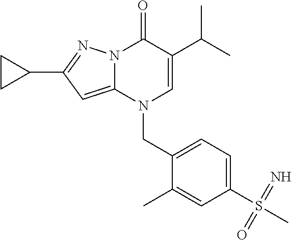 [SMILES: Cc1cc(S(C)(=N)=O)ccc1Cn1cc(C(C)C)c(=O)n2nc(C3CC3)cc12] | ESI-MS m/z = 399.10 [M + H]+; Calculated: 398.2. 1H NMR (400 MHz, DMSO-d6) δ 7.81 (d, J = 2.0 Hz, 1H), 7.76 (s, 1H), 7.67 (dd, J = 8.1, 2.0 Hz, 1H), 6.86 (d, J = 8.1 Hz, 1H), 5.92 (s, 1H), 5.32 (s, 2H), 4.15 (s, 1H), 3.05-3.01 (m, 4H), 2.43 (s, 3H), 1.93 (t, J = 4.6 Hz, 1H), 1.17 (d, J = 6.9 Hz, 6H), 0.92 (dt, J = 8.5, 3.2 Hz, 2H), 0.73 (dd, J = 4.9, 2.3 Hz, 2H). |
| 62 | 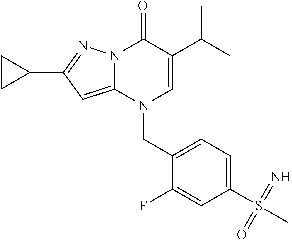 [SMILES: CC(C)c1cn(Cc2ccc(S(C)(=N)=O)cc2F)c2cc(C3CC3)nn2c1=O] | ESI-MS m/z = 403.15 [M + H]+; Calculated: 402.2. 1H NMR (400 MHz, DMSO-d6) δ 7.87 (s, 1H), 7.78-7.71 (m, 2H), 7.47 (t, J = 7.6 Hz, 1H), 6.01 (s, 1H), 5.38 (s, 2H), 4.38 (s, 1H), 3.09 (d, J = 1.2 Hz, 3H), 3.00 (p, J = 6.9 Hz, 1H), 1.94 (td, J = 8.5, 4.3 Hz, 1H), 1.18 (d, J = 6.9 Hz, 6H), 0.97-0.90 (m, 2H), 0.76- 0.71 (m, 2H). 19F NMR (377 MHz, DMSO) δ −114.47. |
| 63 | 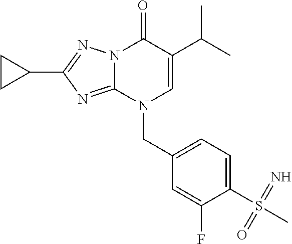 [SMILES: CC(C)c1cn(Cc2ccc(S(C)(=N)=O)c(F)c2)c2nc(C3CC3)nn2c1=O] | ESI-MS m/z = 404.20 [M + H]+; Calculated:403.1. 1H NMR (400 MHz, DMSO-d6) δ 8.02 (s, 1H), 7.89-7.81 (m, 1H), 7.52-7.44 (m, 1H), 7.41-7.34 (m, 1H), 5.41 (s, 2H), 4.71 (s, 1H), 3.19-3.14 (m, 3H), 3.06-2.92 (m, 1H), 2.11-1.99 (m, 1H), 1.19 (d, J = 6.9 Hz, 3H), 1.19 (d, J = 6.9 Hz, 3H), 1.06-0.95 (m, 2H), 0.97-0.85 (m, 2H). |
| 64 | 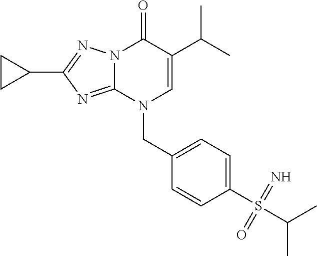 [SMILES: CC(C)c1cn(Cc2ccc(S(=N)(=O)C(C)C)cc2)c2nc(C3CC3)nn2c1=O] | ESI-MS m/z = 414.20 [M + H]+; Calculated: 413.2. 1H NMR (400 MHz, DMSO-d6) δ 8.02 (s, 1H), 7.90-7.77 (m, 2H), 7.64-7.48 (m, 2H), 5.55-5.32 (m, 2H), 4.19-4.05 (m, 1H), 3.26- 3.14 (m, 1H), 3.05-2.92 (m, 1H), 2.11-1.96 (m, 1H), 1.25- 1.15 (m, 6H), 1.14-1.05 (m, 6H), 1.03-0.94 (m, 2H), 0.92-0.82 (m, 2H). |
| 65 | 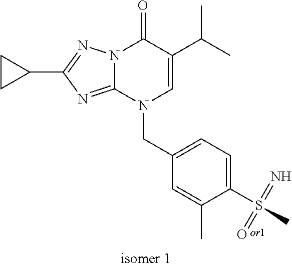 [SMILES: Cc1cc(Cn2cc(C(C)C)c(=O)n3nc(C4CC4)nc23)ccc1[S@](C)(=N)=O] | ESI-MS m/z = 400.15 [M + H]+; Calculated: 399.2. 1H NMR (400 MHz, DMSO-d6) δ 8.01 (s, 1H), 7.93 (d, J = 8.1 Hz, 1H), 7.39 (d, J = 1.9 Hz, 1H), 7.35 (d, J = 8.1 Hz, 1H), 5.35 (s, 2H), 4.26 (s, 1H), 3.05 (s, 3H), 2.99 (p, J = 6.9 Hz, 1H), 2.65 (s, 3H), 2.04 (tt, J = 8.3, 5.0 Hz, 1H), 1.19 (d, J = 6.9 Hz, 6H), 1.05-0.97 (m, 2H), 0.90 (dt, J = 4.7, 3.0 Hz, 2H). |
| 66 | 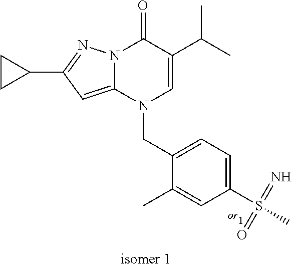 [SMILES: Cc1cc([S@@](C)(=N)=O)ccc1Cn1cc(C(C)C)c(=O)n2nc(C3CC3)cc12] | ESI-MS m/z = 399.15 [M + H]+; Calculated: 398.2. 1H NMR (400 MHz, DMSO-d6) δ 7.81 (s, 1H), 7.76 (s, 1H), 7.67 (d, J = 8.0 Hz, 1H), 6.86 (d, J = 8.1 Hz, 1H), 5.92 (s, 1H), 5.32 (s, 2H), 4.14 (s, 1H), 3.06-3.00 (m, 4H), 2.42 (s, 3H), 1.93 (td, J = 8.4, 4.3 Hz, 1H), 1.17 (d, J = 6.9 Hz, 6H), 0.96-0.88 (m, 2H), 0.75-0.69 (m, 2H). |
| 67 | 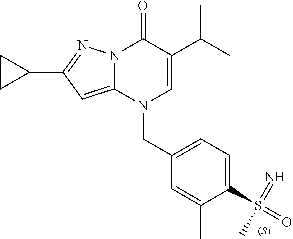 | ESI-MS m/z = 399.20 [M + H]+; Calculated: 398.2. 1H NMR (400 MHz, DMSO-d6) δ 7.94 (d, J = 8.1 Hz, 2H), 7.38- 7.27 (m, 2H), 6.03 (s, 1H), 5.26 (s, 2H), 4.27 (s, 1H), 3.05 (d, J = 1.2 Hz, 3H), 3.01 (q, J = 6.9 Hz, 1H), 2.66 (s, 3H), 1.99-1.88 (m, 1H), 1.20 (d, J = 6.9 Hz, 6H), 0.95 (s, 2H), 0.79-0.72 (m, 2H). |
| 68 | 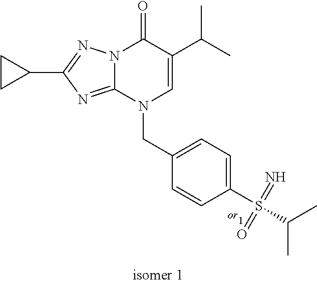 [SMILES: CC(C)c1cn(Cc2ccc([S@@](=N)(=O)C(C)C)cc2)c2nc(C3CC3)nn2c1=O] | ESI-MS m/z = 414.05 [M + H]+; Calculated:413.2. 1H NMR (400 MHz, DMSO-d6) δ 8.03 (s, 1H), 7.89-7.79 (m, 2H), 7.56 (d, J = 8.2 Hz, 2H), 5.43 (s, 2H), 4.14 (s, 1H), 3.27-2.92 (m, 2H), 2.09-1.98 (m, 1H), 1.19 (d, J = 6.9 Hz, 6H), 1.14-1.06 (m, 6H), 1.03-0.96 (m, 2H), 0.92- 0.83 (m, 2H). |
| 69 | 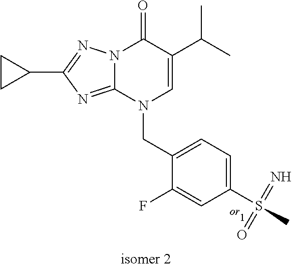 [SMILES: CC(C)c1cn(Cc2ccc([S@](C)(=N)=O)cc2F)c2nc(C3CC3)nn2c1=O] | ESI-MS m/z = 403.95 [M + H]+; Calculated: 403.1. 1H NMR (400 MHz, DMSO-d6) δ 7.99 (s, 1H), 7.74 (dd, J = 19.2, 8.9 Hz, 2H), 7.50 (t, J = 7.6 Hz, 1H), 5.46 (s, 2H), 4.39 (s, 1H), 3.09 (s, 3H), 3.04-2.94 (m, 1H), 2.08-1.96 (m, 1H), 1.19 (d, J = 6.9 Hz, 6H), 1.03-0.94 (m, 2H), 0.90-0.79 (m, 2H). 19F NMR (377 MHz, DMSO) δ −114.96. |
| 70 | 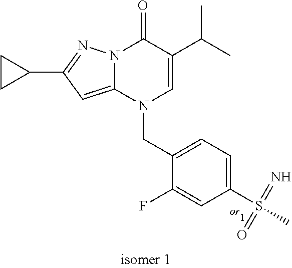 [SMILES: CC(C)c1cn(Cc2ccc([S@@](C)(=N)=O)cc2F)c2cc(C3CC3)nn2c1=O] | ESI-MS m/z = 403.15 [M + H]+; Calculated: 402.2. 1H NMR (400 MHz, DMSO-d6) δ 7.86 (s, 1H), 7.79-7.70 (m, 2H), 7.47 (t, J = 7.6 Hz, 1H), 6.01 (s, 1H), 5.38 (s, 2H), 4.36 (s, 1H), 3.09 (d, J = 1.1 Hz, 3H), 3.01- 2.97 (m, 1H), 1.94 (tt, J = 8.3, 4.9 Hz, 1H), 1.18 (d, J = 6.9 Hz, 6H), 0.98-0.89 (m, 2H), 0.78-0.70 (m, 2H). 19F NMR (376 MHz, DMSO) δ −114.48. |
| 71 | 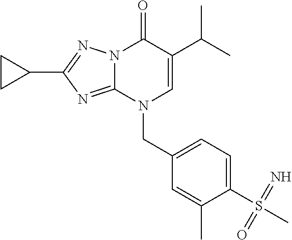 [SMILES: Cc1cc(Cn2cc(C(C)C)c(=O)n3nc(C4CC4)nc23)ccc1S(C)(=N)=O] | ESI-MS m/z = 440.10 [M + H]+; Calculated: 399.2. 1H NMR (400 MHz, DMSO-d6) δ 8.01 (s, 1H), 7.94 (d, J = 8.1 Hz, 1H), 7.40 (s, 1H), 7.35 (d, J = 8.2 Hz, 1H), 5.36 (s, 2H), 4.33 (s, 1H), 3.06 (s, 3H), 2.99 (p, J = 6.9 Hz, 1H), 2.65 (s, 3H), 2.04 (tt, J = 8.6, 4.9 Hz, 1H), 1.19 (d, J = 6.9 Hz, 6H), 1.04-0.98 (m, 2H), 0.94-0.88 (m, 2H). |
| 72 | 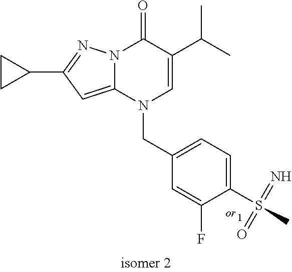 [SMILES: CC(C)c1cn(Cc2ccc([S@](C)(=N)=O)c(F)c2)c2cc(C3CC3)nn2c1=O] | ESI-MS m/z = 403.25 [M + H]+; Calculated:402.2. 1H NMR (400 MHz, DMSO-d6) δ 7.93 (s, 1H), 7.89-7.80 (m, 1H), 7.49-7.41 (m, 1H), 7.36-7.29 (m, 1H), 6.04 (s, 1H), 5.29 (s, 2H), 4.70 (d, J = 1.5 Hz, 1H), 3.16 (d, J = 1.4 Hz, 3H), 3.06-2.93 (m, 1H), 1.99-1.88 (m, 1H), 1.20 (d, J = 6.9 Hz, 3H), 1.20 (d, J = 6.9 Hz, 3H), 0.98-0.88 (m, 2H), 0.79- 0.70 (m, 2H). 19F NMR (376 MHz, DMSO-d6) δ −109.39. |
| 73 Rac | 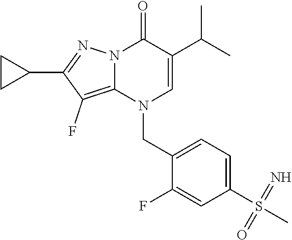 [SMILES: CC(C)c1cn(Cc2ccc(S(C)(=N)=O)cc2F)c2c(F)c(C3CC3)nn2c1=O] | ESI-MS m/z = 421.10 [M + H]+; Calculated: 420.1. 1H NMR (400 MHz, DMSO-d6) δ 7.89 (s, 1H), 7.80-7.72 (m, 2H), 7.40-7.36 (m, 1H), 5.49 (s, 2H), 4.39 (s, 1H), 3.10 (s, 3H), 3.03- 2.98 (m, 1H), 1.98-1.93 (m, 1H), 1.18 (d, J = 6.9 Hz, 6H), 1.00- 0.96 (m, 2H), 0.90-0.87 (m, 2H). 19F NMR (377 MHz, DMSO) δ −115.54, −115.55 |
| 73A | 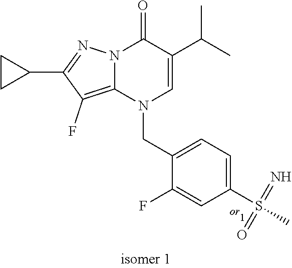 [SMILES: CC(C)c1cn(Cc2ccc([S@@](C)(=N)=O)cc2F)c2c(F)c(C3CC3)nn2c1=O] | ESI-MS m/z = 421.10 [M + H]+; Calculated: 420.1 1H NMR (400 MHz, DMSO-d6) δ 7.89 (s, 1H), 7.78 (dd, J = 9.8, 1.8 Hz, 1H), 7.73 (dd, J = 8.0, 1.8 Hz, 1H), 7.38 (t, J = 7.7 Hz, 1H), 5.49 (s, 2H), 4.40 (s, 1H), 3.10 (d, J = 1.1 Hz, 3H), 2.99 (p, J = 6.9 Hz, 1H), 1.95 (tt, J = 8.3, 5.0 Hz, 1H), 1.18 (d, J = 6.9 Hz, 6H), 1.01- 0.95 (m, 2H), 0.90-0.85 (m, 2H). 19F NMR (377 MHz, DMSO) δ −115.52, −188.47, −188.48. |

### Dosage Forms, Medicaments and Pharmaceuticals

The compounds, molecules or agents of the disclosure may be used to treat (e.g. cure, alleviate or prevent) one or more diseases, conditions or disorders. Thus, in accordance with the disclosure, the compounds and molecules may be manufactured into medicaments or may be incorporated or formulated into pharmaceutical compositions.

References to compositions throughout the disclosure, may be pharmaceutical compositions. For example, any compositions of the disclosure may be formulated as a pharmaceutical composition comprising a pharmaceutically acceptable carrier.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting ENPP1. Diseases or disorders which may benefit from ENPP1 inhibition include, for example, mineralisation, calcium handling or calcification-related disorders, such as hypophosphatasia (HPP).

Further diseases that may be treated with the compounds, molecules and compositions of this disclosure include cancers (also referred to as oncologic diseases).

When administered to a subject, a compound of the disclosure is suitably administered as a component of a composition that comprises a pharmaceutically acceptable carrier or vehicle. One or more additional pharmaceutically acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

The invention will now be described by way of the following non-limiting examples and the accompanying drawings.

### EXAMPLES

### Materials and Methods

Sample preparation: Powders were solubilized in DMSO-de, vortexed vigorously until the solution was clear and transferred to a NMR tube for data acquisition.

NMR spectroscopy:

Liquid-state NMR experiments were recorded on a 400 MHZ (9.4 Tesla) Bruker Avance NEO 400 MHz NMR spectrometer (400 MHz for 1H, 100 MHz for ¹³C) using PI HR-BBO400S1-BBF/H/D-5.0-Z SP probe (Bruker Biospin, Germany).

Liquid-state NMR experiments were recorded on a 300 MHZ (7.05 Tesla) Bruker AVANCE III HD 300 MHz NMR spectrometer (300 MHz for 1H, 75 MHz for ¹³C) using PA BBO 300S1 BBF-H-D-05 Z probe (Bruker Biospin, Germany).

All the experiments used for the resonance assignment procedure and the elucidation of the products structure (1D ¹H, 2D ¹H-¹H-COSY, 2D ¹H-¹H-ROESY, 2D ¹H-13C-HSQC, 2D ¹H-¹³C-HMBC) were recorded at 298 K. ¹H chemical shifts are reported in δ (ppm) as s (singlet), d (doublet), t (triplet), q (quartet), dd (double doublet), m (multiplet) or br s (broad singlet).

### LCMS Chromatography:

LCMS chromatography were recorded with the following methods:

### Method A: 5-100AB, Shimadzu

Instrument: SHIMADZU LCMS-2020; Column: Express C18 3.0*30 mm, 2.7 μm; eluent A: water+0.05 vol % trifluoroacetic acid; eluent B: acetonitrile+0.05 vol % trifluoroacetic acid; gradient: 0-1.2 min, 5-100% B, 1.2-1.8 min 100% B; flow: 1.5 ml/min; temperature: 40° C.; PDA: 220 nm & 254 nm.

### Method B: 5-100AB, Shimadzu

Instrument: SHIMADZU LCMS-2020; Column: Express C18 3.0*30 mm, 2.7 μm; eluent A: water+0.05 vol % trifluoroacetic acid; eluent B: acetonitrile+0.05 vol % trifluoroacetic acid; gradient: 0-2.1 min, 5-100% B, 2.1-2.8 min 100% B; flow: 1.5 ml/min; temperature: 40° C.; PDA: 220 nm & 254 nm.

### Method C: 5-100AB, Shimadzu

Instrument: SHIMADZU LCMS-2020; Column: Express C18 3.0*30 mm, 2.7 μm; eluent A: water+0.1 vol % formic acid; eluent B: acetonitrile+0.07 vol % formic acid; gradient: 0-0.7 min, 5-100% B, 0.7-1.1 min 100% B; flow: 1.5 ml/min; temperature: 40° C.; PDA: 220 nm & 254 nm.

### Method D: 5-100AB, Shimadzu

Instrument: SHIMADZU LCMS-2020; Column: Express C18 3.0*30 mm, 2.7 μm; eluent A: water+0.1 vol % formic acid; eluent B: acetonitrile+0.07 vol % formic acid; gradient: 0-1.2 min, 5-100% B, 1.2-1.8 min 100% B; flow: 1.5 ml/min; temperature: 40° C.; PDA: 220 nm & 254 nm.

### Method E: 10-95AB, Shimadzu

Instrument: SHIMADZU LCMS-2020; Column: EVO C18 3.0*30 mm, 2.6 μm; eluent A: water+0.04 vol % ammonia; eluent B: acetonitrile; gradient: 0-1.2 min, 10-95% B, 1.2-1.8 min 95% B; flow: 1.2 ml/min; temperature: 40° C.; PDA: 220 nm & 254 nm.

### Method F: 10-95AB, Shimadzu

Instrument: SHIMADZU LCMS-2020; Column: EVO C18 3.0*30 mm, 2.6 μm; eluent A: water+0.04 vol % ammonia; eluent B: acetonitrile; gradient: 0-0.7 min, 10-95% B, 0.7-1.1 min 95% B; flow: 1.2 ml/min; temperature: 40° C.; PDA: 220 nm & 254 nm.

### Method G: 10-95AB, Shimadzu

Instrument: SHIMADZU LCMS-2020; Column: EVO C18 3.0*50 mm, 2.6 μm; eluent A: water+5 Mm Ammonium bicarbonate; eluent B: acetonitrile; gradient: 0-1.2 min, 10-95% B, 1.2-1.8 min 95% B; flow: 1.2 ml/min; temperature: 40° C.; PDA: 220 nm & 254 nm.

Temperatures are given in degrees Celsius (° C.). The reactants used in the examples below may be obtained from commercial sources or they may be prepared from commercially available starting materials as described herein or by methods known in the art. All of the compounds of the invention are synthesised according to the Examples described herein. The progress of the reactions described herein were followed as appropriate by e.g. LC, GC or TLC, and as the skilled person will readily realise, reaction times and temperatures may be adjusted accordingly.

### Abbreviations

In addition to the definitions above, the following abbreviations are used in the synthetic schemes above and the examples below. If an abbreviation used herein is not defined, it has its generally accepted meaning:
Bn benzyl
RPA-088 Benzyl bromide
BnBr
CH₃CN Acetonitrile
CuCl₂ Copper chloride
DCM Dichloromethane
DIPEA Diisopropylethylamine
DMSO Dimethylsulfoxide
EA Ethyl Acetate
Et Ethyl
EtOAc Ethyl acetate
Et₃N Triethylamine
EtOH Ethanol
Et₂O Diethyl ether
h hour
H₂O water
HBr Hydrobromic acid
HCl Hydrochloric acid
I₂ Iodine
K₂CO₃ Potassium carbonate
KOAc Potassium acetate
KOtBu Potassium tert-butoxide
LiOH Lithium hydroxide
min minutes
Me Methyl
MeCN Acetonitrile
MeI Methyl iodide
MeMgBr Methyl magnesium bromide
MeO or OMe Methoxy
MeOH Methanol
MgSO₄ Magnesium sulfate
MS Mass spectrometry
N₂ Nitrogen
NaCl Sodium chloride
NaH Sodium hydride
NaOAc Sodium acetate
NaOH Sodium hydroxide
Na₂CO₃ Sodium carbonate
Na₂SO₄ Sodium sulfate
NaH Sodium hydride
NaHCO₃Sodium bicarbonate
NH₄Cl Ammonium chloride
Pd₂(dba)₃ Tris(dibenzylideneacetone)dipalladium (0)
PE Petroleum Ether
POCl₃ Phosphoryl chloride
Prep-HPLC
Preparative high performance liquid chromatography
Prep-TLC Preparative thin layer chromatography
Ph Phenyl
rt Room temperature (18 to 22° C.)
SiO₂ Silica gel
t-BuOH Tert-butanol
t-BuOK Sodium tert-butoxide
THF Tetrahydrofuran

### Example 1—Compounds

### Compound 1: 6-isopropyl-2-methyl-4-(1S,4S,5S)-2-(S-methylsulfonimidoyl)-2-azabicyclo[2.2.1]heptan-5-yl)methyl)pyrazolo[1,5-a]pyrimidin-7(4H)-one

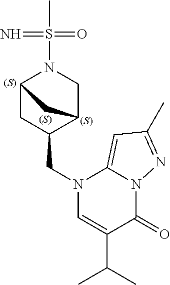

Step 1: Tert-butyl(1S,4S,5S)-5-({6-isopropyl-2-methyl-7-oxopyrazolo[1,5-a]pyrimidin-4-yl}methyl)-2-azabicyclo[2.2.1]heptane-2-carboxylate

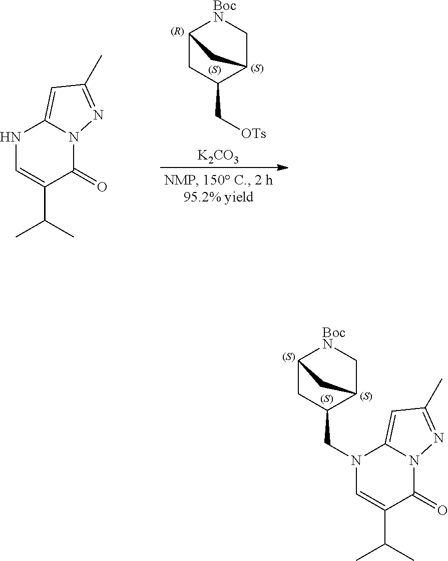

To a stirred solution of 6-isopropyl-2-methyl-4H-pyrazolo[1,5-a]pyrimidin-7-one (276 mg, 1.44 mmol, 1.1 equiv) and K₂CO₃ (543 mg, 3.93 mmol, 3.0 equiv) in NMP (10 mL) was added tert-butyl(1R,4S,5S)-5-[[(4-methylbenzenesulfonyl)oxy]methyl)-2-azabicyclo[2.2.1]heptane-2-carboxylate (500 mg, 1.31 mmol, 1.0 equiv) at 25° C. under nitrogen atmosphere. The resulting mixture was stirred for 2 h at 150° C. under nitrogen atmosphere. The reaction was monitored by LCMS. The mixture was allowed to cool down to 25° C. The resulting mixture was diluted with EtOAc (100 mL). The resulting mixture was washed with 3×100 ml of water. The resulting mixture was concentrated under vacuum. The residue was purified by reversed-phase flash chromatography with the following conditions: column: C18; mobile phase: MeCN in Water (0.1% FA), 10% to 50% gradient in 30 min; detector: UV 254 nm. This resulted in tert-butyl(1S,4S,5S)-5-({6-isopropyl-2-methyl-7-oxopyrazolo[1,5-a]pyrimidin-4-yl}methyl)-2-azabicyclo[2.2.1]heptane-2-carboxylate.

ESI-MS m/z=401.25 [M+H]+; Calculated: 400.2.

1H NMR (400 MHZ, DMSO-d6) δ 7.75 (s, 1H), 6.21 (s, 1H), 4.10-4.03 (m, 1H), 4.01-3.92 (m, 1H), 3.91-3.80 (m, 1H), 3.15-3.10 (m, 1H), 3.05-2.95 (m, 1H), 2.88-2.82 (m, 1H), 2.30 (s, 3H), 2.21 (m, 1H), 1.81-1.77 (m, 1H), 1.68-1.64 (m, 1H), 1.57-1.39 (m, 2H), 1.37-1.35 (m, 9H), 1.31-1.24 (m, 1H), 1.18 (d, J=6.9 Hz, 6H).

### Step 2: 4-[(1R,4S,5S)-2-azabicyclo[2.2.1]heptan-5-ylmethyl]-6-isopropyl-2-methylpyrazolo[1,5-a]pyrimidin-7-one

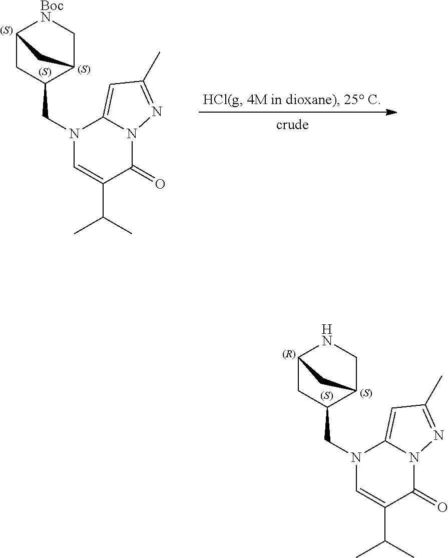

A solution of tert-butyl(1S,4S,5S)-5-({6-isopropyl-2-methyl-7-oxopyrazolo[1,5-a]pyrimidin-4-yl}methyl)-2-azabicyclo[2.2.1]heptane-2-carboxylate (250 mg, 0.62 mmol, 1.0 equiv) in HCl (gas) in 1,4-dioxane (5 mL) was stirred for 1 h at 25° C. under nitrogen atmosphere. The reaction was monitored by LCMS. The resulting mixture was concentrated under reduced pressure. The resulting mixture was diluted with water (50 mL). The mixture was neutralised to pH 8 with saturated NaHCO₃(aq.), extracted with CHCl3/IPA (3/1) (3×50 mL). The combined organic layers were washed with brine (1×150 mL), and dried over anhydrous Na₂SO₄. After filtration, the filtrate was concentrated under reduced pressure. This resulted in 4-[(1R,4S,5S)-2-azabicyclo[2.2.1]heptan-5-ylmethyl]-6-isopropyl-2-methylpyrazolo[1,5-a]pyrimidin-7-one (crude).

ESI-MS m/z=301.25 [M+H]+; Calculated: 300.2.

### Step 3: 6-Isopropyl-2-methyl-4-(((1S,4S,5S)-2-(S-methylsulfonimidoyl)-2-azabicyclo[2.2.1]heptan-5-yl)methyl)pyrazolo[1,5-a]pyrimidin-7(4H)-one

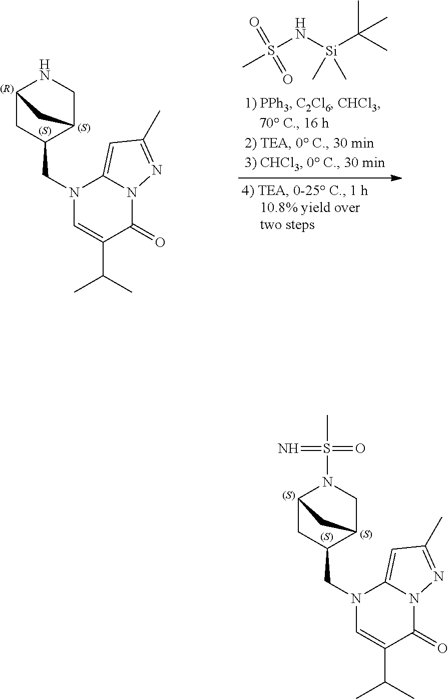

A solution of PPh3 (1.05 g, 4.00 mmol, 6.0 equiv) and 1,1,1,2,2,2-hexachloroethane (946 mg, 4.00 mmol, 6.0 equiv) in CHCl3 (2.00 mL) was stirred for 16 h at 70° C. under nitrogen atmosphere. The mixture was allowed to cool down to 0° C. To the above mixture was added TEA (606 mg, 5.99 mmol, 9.0 equiv) dropwise at 0° C. The resulting mixture was stirred for an additional 15 min at 0° C. To the above mixture was added N-(tert-butyldimethylsilyl)methanesulfonamide (836 mg, 4.00 mmol, 6.0 equiv) in CHCl3 (2 mL) dropwise at 0° C. The resulting mixture was stirred for an additional 30 min at 0° C. To the above mixture was added 4-[(1R,4S,5S)-2-azabicyclo[2.2.1]heptan-5-ylmethyl]-6-isopropyl-2-methylpyrazolo[1,5-a]pyrimidin-7-one (200 mg, 0.67 mmol, 1.0 equiv) in CHCl3 (2 mL) dropwise at 0° C. The resulting mixture was stirred for an additional 1 h at 25° C. The reaction was monitored by LCMS. The resulting mixture was diluted with water (50 mL). The resulting mixture was extracted with CHCl3/IPA (3×50 mL). The combined organic layers were washed with brine (1×150 mL), and dried over anhydrous Na₂SO₄. After filtration, the filtrate was concentrated under reduced pressure. The residue was purified by reverse phase flash with the following conditions (column: C18; mobile phase: MeCN in Water (0.1% FA), 0% to 50% gradient in 30 min; detector: UV 254 nm.) to afford crude product. The crude product (150 mg) was purified by Prep-HPLC with the following conditions (Column: XSelect CSH Fluoro Phenyl 30*150 mm, 5 m; Mobile Phase A: Water (0.1% FA), Mobile Phase B: ACN; Flow rate: 60 mL/min mL/min; Gradient: 6% B to 21% B in 8 min; Wave Length: 254 nm/220 nm; RT1 (min): 10.27), the pure factions were concentrated under vacuum then lyophilized to afford 6-isopropyl-2-methyl-4-(((1S,4S,5S)-2-(S-methylsulfonimidoyl)-2-azabicyclo[2.2.1]heptan-5-yl)methyl)pyrazolo[1,5-a]pyrimidin-7(4H)-one.

ESI-MS m/z=378.15 [M+H]+; Calculated: 377.2.

1H NMR (400 MHZ, DMSO-d6) δ 7.76 (s, 1H), 6.21 (s, 1H), 4.09-3.75 (m, 3H), 3.18-2.94 (m, 2H), 2.81-2.68 (m, 4H), 2.36-2.23 (m, 5H), 1.87-1.72 (m, 2H), 1.57-1.44 (m, 1H), 1.30-1.22 (m, 1H), 1.18 (d, J=6.9 Hz, 6H).

Total proton count from structure: 27.

Total proton count from spectrum: 26 (exchangeable hydrogen did not appear).

### Compound 2: (1S,4S,5S)-5-((2-methyl-7-oxo-6-(pyridin-3-yl)-[1,2,4]triazolo[1,5-a]pyrimidin-4(7H)-yl)methyl)-2-azabicyclo[2.2.1]heptane-2-sulfonamide

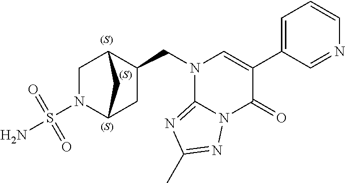

### Step 1: Methyl 3-oxo-2-(pyridin-3-yl) propanoate

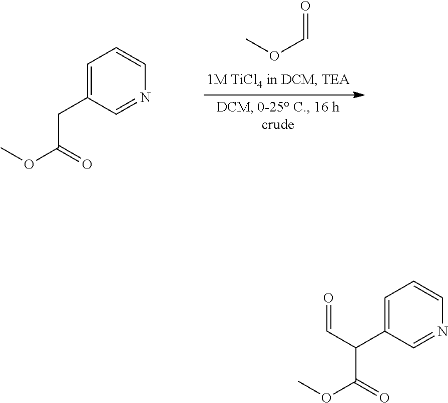

To a stirred solution of methyl 2-(pyridin-3-yl)acetate (10.00 g, 66.2 mmol, 1.0 equiv) and methyl formate (11.92 g, 198.5 mmol, 3.0 equiv) in DCM (200 mL) was added 1M TiCl₄ in DCM (132 mL, 132.3 mmol, 2.0 equiv) and TEA (16.07 g, 158.8 mmol, 2.4 equiv) dropwise at 0° C. under nitrogen atmosphere. The resulting mixture was stirred for an additional 16 h at 25° C. The desired product could be detected by LCMS. The resulting mixture was poured into water (500 mL) at 0° C. The resulting mixture was extracted with EtOAc (3×500 mL). The combined organic layers were washed with brine (1 L) and dried over anhydrous Na₂SO₄. After filtration, the filtrate was concentrated under reduced pressure to afford methyl 3-oxo-2-(pyridin-3-yl) propanoate (crude).

ESI-MS m/z=180.10 [M+H]⁺; Calculated: 179.1.

### Step 2: 2-Methyl-6-(pyridin-3-yl)-[1,2,4]triazolo[1,5-a]pyrimidin-7(4H)-one

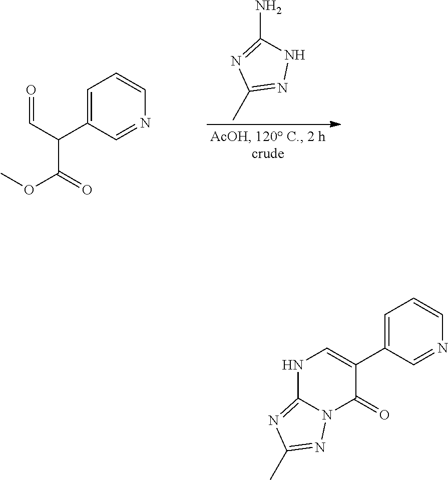

A solution of methyl 3-oxo-2-(pyridin-3-yl) propanoate (2.50 g, 14.0 mmol, 1.0 equiv) and 5-methyl-2H-1,2,4-triazol-3-amine (1.51 g, 15.4 mmol, 1.1 equiv) in AcOH (20 mL) was stirred for 2 h at 120° C. under nitrogen atmosphere. The desired product could be detected by LCMS. The mixture was allowed to cool down to 25° C. The resulting mixture was concentrated under reduced pressure. The residue was purified by trituration with EtOAc (10 mL) to afford 2-methyl-6-(pyridin-3-yl)-[1,2,4]triazolo[1,5-a]pyrimidin-7(4H)-one (crude).

ESI-MS m/z=227.95 [M+H]⁺; Calculated: 227.1.

### Step 3: tert-butyl(1S,4S,5S)-5-((2-methyl-7-oxo-6-(pyridin-3-yl)-[1,2,4]triazolo[1,5-a]pyrimidin-4(7H)-yl)methyl)-2-azabicyclo[2.2.1]heptane-2-carboxylate

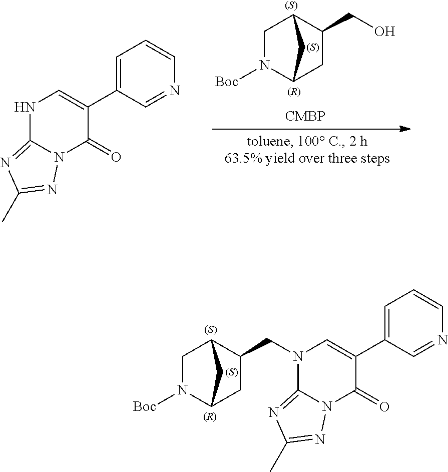

To a stirred solution of 2-methyl-6-(pyridin-3-yl)-[1,2,4]triazolo[1,5-a]pyrimidin-7(4H)-one (330 mg, 1.45 mmol, 1.1 equiv) and tert-butyl(1R,4S,5S)-5-(hydroxymethyl)-2-azabicyclo[2.2.1]heptane-2-carboxylate (300 mg, 1.32 mmol, 1.0 equiv) in Toluene (6 mL) was added 2-(tributyl-lambda5-phosphanylidene) acetonitrile (1.59 g, 6.60 mmol, 5.0 equiv) at 25° C. The resulting mixture was stirred for 2 h at 100° C. under nitrogen atmosphere. The desired product could be detected by LCMS. The mixture was allowed to cool down to 25° C. The resulting mixture was concentrated under reduced pressure. The residue was purified by reversed-phase flash chromatography with the following conditions: column: C18; mobile phase: MeCN in Water (10 mmol/L NH₄HCO₃), 30% to 70% gradient in 30 min; detector: UV 254 nm to afford tert-butyl(1S,4S,5S)-5-((2-methyl-7-oxo-6-(pyridin-3-yl)-[1,2,4]triazolo[1,5-a]pyrimidin-4(7H)-yl)methyl)-2-azabicyclo[2.2.1]heptane-2-carboxylate.

ESI-MS m/z=437.15 [M+H]⁺; Calculated: 436.2.

¹H NMR (400 MHZ, DMSO-d₆) δ 8.85 (d, J=2.3 Hz, 1H), 8.60-8.50 (m, 2H), 8.12-8.07 (m, 1H), 7.52-7.44 (m, 1H), 4.25-4.13 (m, 1H), 4.11-3.97 (m, 2H), 3.17-3.05 (m, 1H), 2.85 (d, J=9.6 Hz, 1H), 2.46-2.33 (m, 5H), 1.78-1.65 (m, 2H), 1.59-1.48 (m, 1H), 1.42-1.29 (m, 10H).

### Step 4: 4-(((1R,4S,5S)-2-Azabicyclo[2.2.1]heptan-5-yl)methyl)-2-methyl-6-(pyridin-3-yl)-[1,2,4]triazolo[1,5-a]pyrimidin-7(4H)-one HCl salt

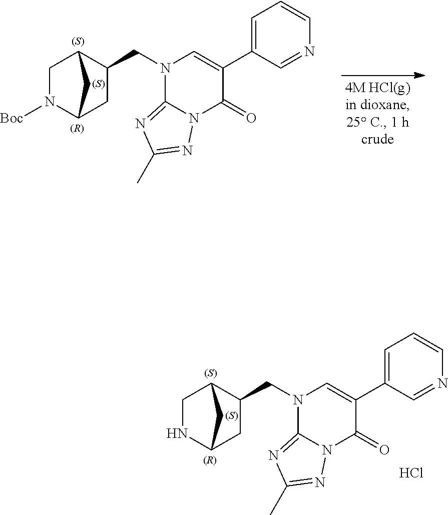

A solution of tert-butyl(1S,4S,5S)-5-((2-methyl-7-oxo-6-(pyridin-3-yl)-[1,2,4]triazolo[1,5-a]pyrimidin-4(7H)-yl)methyl)-2-azabicyclo[2.2.1]heptane-2-carboxylate (250 mg, 0.57 mmol, 1.0 equiv) in 4M HCl (gas) in 1,4-dioxane (5 mL) was stirred for 1 h at 25° C. The desired product could be detected by LCMS. The resulting mixture was concentrated under reduced pressure to afford 4-(((1R,4S,5S)-2-azabicyclo[2.2.1]heptan-5-yl)methyl)-2-methyl-6-(pyridin-3-yl)-[1,2,4]triazolo[1,5-a]pyrimidin-7(4H)-one HCl salt (crude).

ESI-MS m/z=337.15 [M+H]⁺; Calculated: 372.1.

### Step 5: (1S,4S,5S)-5-((2-Methyl-7-oxo-6-(pyridin-3-yl)-[1,2,4]triazolo[1,5-a]pyrimidin-4(7H)-yl)methyl)-2-azabicyclo[2.2.1]heptane-2-sulfonamide

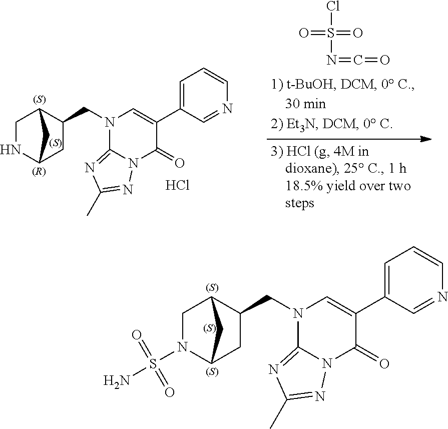

To a stirred solution of chlorosulfonyl isocyanate (456 mg, 3.22 mmol, 6.0 equiv) in DCM (2.8 mL) was added t-BuOH (358 mg, 4.82 mmol, 9.0 equiv) dropwise at 0° C. under nitrogen atmosphere. The resulting mixture was stirred for 30 min at 0° C. under nitrogen atmosphere. A stirred solution of 4-(((1R,4S,5S)-2-azabicyclo[2.2.1]heptan-5-yl)methyl)-2-methyl-6-(pyridin-3-yl)-[1,2,4]triazolo[1,5-a]pyrimidin-7(4H)-one HCl salt (200 mg, 0.55 mmol, 1.0 equiv) in DCM (3 mL) was treated with Et₃N (543 mg, 5.36 mmol, 10.0 equiv) for 5 min at 25° C. under nitrogen atmosphere followed by the addition of above mixture dropwise at 0° C. The resulting mixture was stirred for 1 h at 0° C. under nitrogen atmosphere. The desired product could be detected by LCMS. The resulting mixture was concentrated under reduced pressure. To the above mixture was added 4M HCl (gas) in 1,4-dioxane (5 mL) over 1 min at 25° C. The resulting mixture was stirred for an additional 1 h at 25° C. The desired product could be detected by LCMS. The resulting mixture was concentrated under reduced pressure. The resulting mixture was diluted with water (100 mL). The mixture was neutralized to pH 7 with NaOH(aq.). The resulting mixture was extracted with CH₂Cl₂/IPA (3×100 mL). The combined organic layers were washed with brine (200 mL), and dried over anhydrous Na₂SO₄. After filtration, the filtrate was concentrated under reduced pressure. The crude product (150 mg) was purified by Prep-HPLC with the following conditions (Column: XSelect CSH Prep C18 OBD Column, 30*150 mm, 5 m; Mobile Phase A: Water (0.1% FA), Mobile Phase B: ACN; Flow rate: 60 mL/min; Gradient: 2% B to 13% B in 9 min; Wave Length: 254 nm/220 nm nm; RT1 (min): 8.18), the pure fraction was concentrated then lyophilized to afford (1S,4S,5S)-5-((2-methyl-7-oxo-6-(pyridin-3-yl)-[1,2,4]triazolo[1,5-a]pyrimidin-4(7H)-yl)methyl)-2-azabicyclo[2.2.1]heptane-2-sulfonamide.

ESI-MS m/z=416.15 [M+H]⁺; Calculated: 415.1.

¹H NMR (400 MHZ, DMSO-d₆) δ 8.84 (d, J=2.3 Hz, 1H), 8.60-8.52 (m, 2H), 8.13-8.06 (m, 1H), 7.52-7.45 (m, 1H), 6.68 (br, 2H), 4.20-4.11 (m, 1H), 4.06-3.98 (m, 1H), 3.95 (s, 1H), 3.12-3.05 (m, 1H), 2.74 (d, J=9.1 Hz, 1H), 2.48-2.38 (m, 5H), 1.96-1.85 (m, 1H), 1.72-1.57 (m, 2H), 1.37-1.27 (m, 1H).

Total proton count from structure: 21.

Total proton count from spectrum: 21.

### Compound 3 Rac: 2-cyclopropyl-6-isopropyl-4-(4-(S-methylsulfonimidoyl)benzyl)pyrazolo[1,5-a]pyrimidin-7(4H)-one

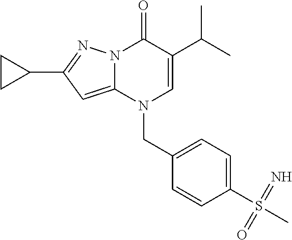 [SMILES: CC(C)c1cn(Cc2ccc(S(C)(=N)=O)cc2)c2cc(C3CC3)nn2c1=O]

### Step 1: 2-Cyclopropyl-6-isopropylpyrazolo[1,5-a]pyrimidin-7(4H)-one

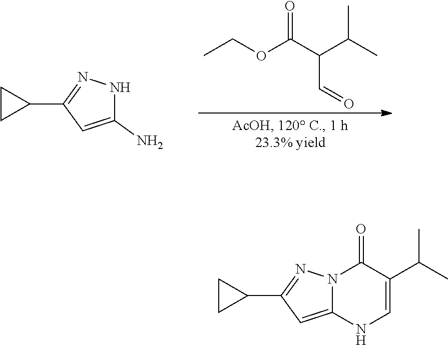 [SMILES: Nc1cc(C2CC2)n[nH]1 ; CCOC(=O)C(C=O)C(C)C ; CC(C)c1c[nH]c2cc(C3CC3)nn2c1=O]

A mixture of ethyl 2-formyl-3-methylbutanoate (500 mg, 3.16 mmol, 1.0 equiv) and 5-cyclopropyl-2H-pyrazol-3-amine (389 mg, 3.16 mmol, 1.0 equiv) in AcOH (4 mL) was stirred for 1 h at 120° C. under nitrogen atmosphere. The desired product could be detected by LCMS. The resulting mixture was poured into water (30 mL). The resulting mixture was extracted with EtOAc (3×30 mL). The combined organic layers were washed with brine (15 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (10 mmol/L NH₄HCO₃), 15% to 45% gradient in 20 min; detector: UV 220 nm to afford 2-cyclopropyl-6-isopropylpyrazolo[1,5-a]pyrimidin-7(4H)-one.

ESI-MS m/z=218.15 [M+H]⁺; Calculated: 217.1.

¹H NMR (400 MHZ, DMSO-d₆) δ 7.57 (s, 1H), 5.81 (s, 1H), 3.05-2.90 (m, 1H), 2.03-1.89 (m, 1H), 1.28-1.13 (m, 7H), 0.99-0.88 (m, 2H), 0.81-0.73 (m, 2H).

### Step 2: 2-Cyclopropyl-6-isopropyl-4-(4-(S-methylsulfonimidoyl)benzyl)pyrazolo[1,5-a]pyrimidin-7(4H)-one

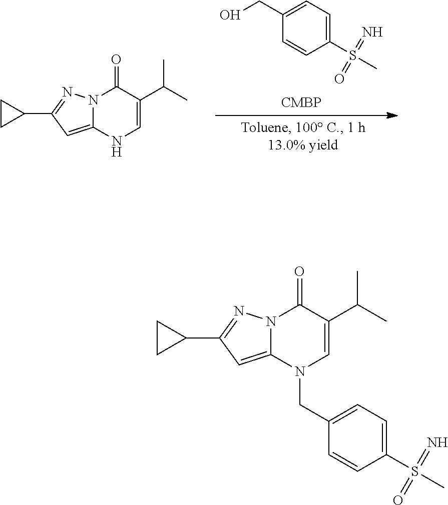 [SMILES: CC(C)c1c[nH]c2cc(C3CC3)nn2c1=O ; CS(=N)(=O)c1ccc(CO)cc1 ; CC(C)c1cn(Cc2ccc(S(C)(=N)=O)cc2)c2cc(C3CC3)nn2c1=O]

To a stirred mixture of 2-cyclopropyl-6-isopropylpyrazolo[1,5-a]pyrimidin-7(4H)-one (160 mg, 0.73 mmol, 1.0 equiv) and [4-(hydroxymethyl)phenyl](imino)methyl-lambda6-sulfanone (150 mg, 0.81 mmol, 1.1 equiv) in Toluene (3 mL) was added CMBP (533 mg, 2.20 mmol, 3.0 equiv) at 25° C. under nitrogen atmosphere. The resulting mixture was stirred for 1 h at 100° C. under nitrogen atmosphere. The desired product could be detected by LCMS. The resulting mixture was concentrated under reduced pressure. The residue was purified by reversed-phase flash chromatography with the following conditions: column: C18; mobile phase: MeCN in Water (10 mmol/L NH₄HCO₃), 16% to 44% gradient in 10 min; detector: UV 220 nm to afford the crude product. The crude product was purified by Prep-HPLC with the following conditions (Column: Sunfire prep C18 column 30*150 mm, 5 m; Mobile Phase A: Water (0.1% FA), Mobile Phase B: MEOH; Flow rate: 60 mL/min mL/min; Gradient: 29% B to 49% B in 10 min; Wave Length: 254 nm/220 nm nm; RT1 (min): 9), the pure fraction was concentrated under reduced pressure then lyophilized to afford 2-cyclopropyl-6-isopropyl-4-(4-(S-methylsulfonimidoyl)benzyl)pyrazolo[1,5-a]pyrimidin-7(4H)-one.

ESI-MS m/z=385.05 [M+H]⁺; Calculated: 384.2.

¹H NMR (400 MHZ, DMSO-d₆) δ 7.98-7.88 (m, 3H), 7.56-7.50 (m, 2H), 6.03 (s, 1H), 5.33 (s, 2H), 4.21 (s, 1H), 3.04 (s, 3H), 3.05-2.94 (m, 1H), 1.99-1.88 (m, 1H), 1.23-1.17 (m, 6H), 0.98-0.87 (m, 2H), 0.78-0.70 (m, 2H).

Total proton count from structure: 24.

Total proton count from spectrum: 24.

### Compound 3A: rel-(R)-2-cyclopropyl-6-isopropyl-4-(4-(S-methylsulfonimidoyl)benzyl)pyrazolo[1,5-a]pyrimidin-7(4H)-one

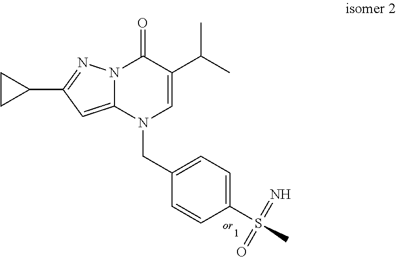 [SMILES: CC(C)c1cn(Cc2ccc([S@](C)(=N)=O)cc2)c2cc(C3CC3)nn2c1=O]

### Step 1: N{[4-(hydroxymethyl)phenyl](methyl)-lambda4-sulfanylidene}-4-methylbenzenesulfonamide

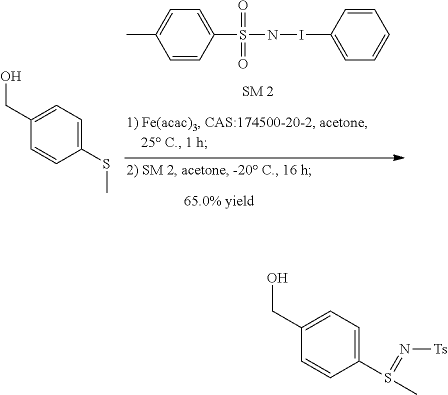

A mixture of 2,6-bis[(4S)-4-phenyl-4,5-dihydro-1,3-oxazol-2-yl]pyridine (CAS: 174500-20-0) (479 mg, 1.29 mmol, 0.20 equiv) and Fe(acac)₃ (457 mg, 1.29 mmol, 0.20 equiv) in acetone (30 mL) was stirred for 1 h at 25° C. under argon atmosphere. To the above mixture was added [4-(methylsulfanyl)phenyl]methanol (1.00 g, 6.48 mmol, 1.0 equiv) in portions over 5 min at 25° C. The resulting mixture was stirred for an additional 5 min at −20° C. To the above mixture was added 4-methyl-N-(phenyl-lambda3-iodanylidene)benzenesulfonamide (2.90 g, 7.78 mmol, 1.2 equiv) in acetone (30 mL) in portions over 5 min at −20° C. The resulting mixture was stirred for an additional 16 h at −20° C. The desired product could be detected by LCMS. The reaction was quenched with water/ice at 0° C. (100 mL). The resulting mixture was extracted with EA (2×60 mL) and DCM/i-PrOH (5:1, 100 mL). The combined organic layers were dried over anhydrous Na₂SO₄. The resulting mixture was concentrated under reduced pressure. The residue was purified by reversed-phase flash chromatography with the following conditions: column: C18; mobile phase: MeCN in Water (0.1% NH₃·H₂O+10 mmol/L NH₄HCO₃), 0% to 30% gradient in 25 min; detector: UV 220 nm. to afford N{[4-(hydroxymethyl)phenyl](methyl)-lambda4-sulfanylidene}-4-methylbenzene-sulfonamide.

ESI-MS m/z=323.90 [M+H]⁺; Calculated: 323.1.

¹H NMR (400 MHZ, DMSO-d₆) δ 7.77-7.70 (m, 2H), 7.62-7.55 (m, 2H), 7.50 (d, J=8.3 Hz, 1H), 7.50 (d, J=8.3 Hz, 1H), 7.26 (d, J=8.0 Hz, 1H), 7.26 (d, J=8.0 Hz, 1H), 5.40 (t, J=5.7 Hz, 1H), 4.56 (d, J=5.7 Hz, 2H), 2.93 (s, 3H), 2.33 (s, 3H).

### Step 2: N-[(4-{[(tert-butyldimethylsilyl)oxy]methyl}phenyl)(methyl)-lambda4-sulfanylidene]-4-methylbenzenesulfonamide

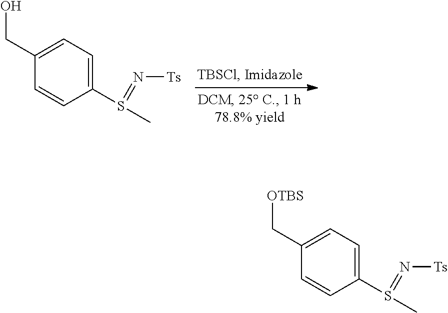 [SMILES: Cc1ccc(S(=O)(=O)/N=S(\C)c2ccc(CO)cc2)cc1 ; Cc1ccc(S(=O)(=O)/N=S(\C)c2ccc(CO[Si](C)(C)C(C)(C)C)cc2)cc1]

To a stirred mixture of TBSCl (1.05 g, 6.95 mmol, 1.5 equiv) and Imidazole (631.47 mg, 9.27 mmol, 2.0 equiv) in DCM (10 mL) was added rel-N-[(R)-[4-(hydroxymethyl)phenyl](methyl)-lambda4-sulfanylidene]-4-methylbenzenesulfonamide (1.50 g, 4.63 mmol, 1.0 equiv) at 25° C. under nitrogen atmosphere. The resulting mixture was stirred for 1 h at 25° C. under nitrogen atmosphere. The desired product could be detected by LCMS. The resulting mixture was diluted with DCM (50 mL). The organic layer was washed with water (3×10 mL), and dried over anhydrous Na₂SO₄. After filtration, the filtrate was concentrated under reduced pressure. The residue was purified by silica gel column chromatography, eluted with PE-EA (0-70% in 15 min) to afford N-[(4-{[(tert-butyldimethylsilyl)oxy]methyl}phenyl)(methyl)-lambda4-sulfanylidene]-4-methylbenzenesulfonamide.

ESI-MS m/z=438.10 [M+H]⁺; Calculated: 437.2.

¹H NMR (400 MHZ, DMSO-d₆) δ 7.79-7.72 (m, 2H), 7.62-7.55 (m, 2H), 7.53-7.46 (m, 2H), 7.28-7.21 (m, 2H), 4.78 (s, 2H), 2.95 (s, 3H), 2.32 (s, 3H), 0.92 (s, 9H), 0.10 (s, 6H).

### Step 3: (4-{[(Tert-butyldimethylsilyl)oxy]methyl}phenyl)(methyl)[(4-methylbenzenesulfonyl)imino]-lambda6-sulfanone

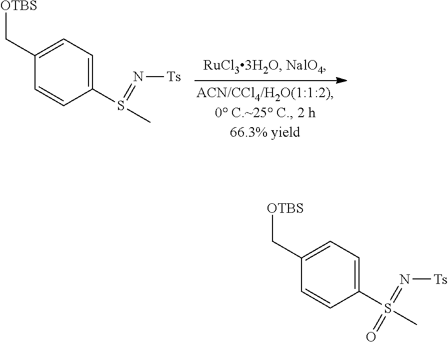 [SMILES: Cc1ccc(S(=O)(=O)/N=S(\C)c2ccc(CO[Si](C)(C)C(C)(C)C)cc2)cc1 ; Cc1ccc(S(=O)(=O)N=S(C)(=O)c2ccc(CO[Si](C)(C)C(C)(C)C)cc2)cc1]

A solution of N-[(R)-(4-{[(tert-butyldimethylsilyl)oxy]methyl}phenyl)(methyl)-lambda4-sulfanylidene]-4-methylbenzenesulfonamide (1.60 g, 3.65 mmol, 1.0 equiv) in CCl₄ (20 mL) and ACN (20 mL) was stirred for 5 min at 0° C. under nitrogen atmosphere. To the above mixture was added RuCl₃·H₂O (82 mg, 0.36 mmol, 0.10 equiv) in H₂O (40 mL) dropwise at 0° C. To the above mixture was added NaIO₄ (1.17 g, 5.48 mmol, 1.5 equiv) in portions at 0° C. The resulting mixture was stirred for an additional 2 h at 25° C. The desired product could be detected by LCMS. The precipitated solids were collected by filtration and washed with EtOAc (3×50 mL). The filtrate was washed with water (3×30 mL), and dried over anhydrous Na₂SO₄. After filtration, the filtrate was concentrated under reduced pressure. The residue was purified by silica gel column chromatography, eluted with CH₂Cl₂/MeOH (0-15% in 20 min) to afford (4-{[(tert-butyl-dimethylsilyl)oxy]methyl}phenyl)(methyl)[(4-methylbenzenesulfonyl)imino]-lambda6-sulfanone.

ESI-MS m/z=454.10 [M+H]⁺; Calculated: 453.1.

¹H NMR (400 MHZ, DMSO-d₆) δ 7.94-7.84 (m, 2H), 7.63-7.60 (m, 2H), 7.56 (d, J=8.3 Hz, 2H), 7.30 (d, J=8.1 Hz, 2H), 4.84 (s, 2H), 3.58 (s, 3H), 2.36 (s, 3H), 0.93 (s, 9H), 0.11 (s, 6H).

### Step 4: [4-(Hydroxymethyl)phenyl](methyl) [(4-methylbenzenesulfonyl)imino]-lambda6-sulfanone

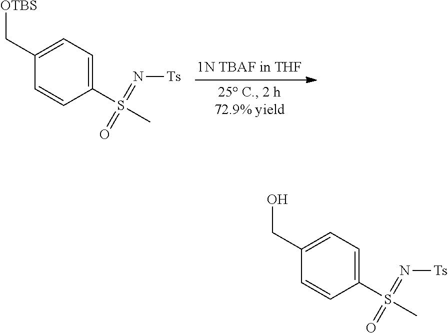 [SMILES: Cc1ccc(S(=O)(=O)N=S(C)(=O)c2ccc(CO[Si](C)(C)C(C)(C)C)cc2)cc1 ; Cc1ccc(S(=O)(=O)N=S(C)(=O)c2ccc(CO)cc2)cc1]

A solution of (4-{[(tert-butyldimethylsilyl)oxy]methyl}phenyl)(methyl)[(4-methylbenzene-sulfonyl)imino]-lambda6-sulfanone (1.10 g, 2.42 mmol, 1.0 equiv) in TBAF (10 mL, 1N in THF) was stirred for 2 h at 25° C. under nitrogen atmosphere. The desired product could be detected by LCMS. The resulting mixture was poured into water (50 mL). The aqueous layer was extracted with EtOAc (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (0-20% in 25 min) to afford [4-(hydroxymethyl)phenyl](methyl) [(4-methylbenzenesulfonyl)imino]-lambda6-sulfanone.

ESI-MS m/z=340.05 [M+H]⁺; Calculated: 339.1.

¹H NMR (400 MHZ, DMSO-d₆) δ 7.92-7.86 (m, 2H), 7.60 (s, 4H), 7.34-7.28 (m, 2H), 4.62 (s, 2H), 3.57 (s, 3H), 2.36 (s, 3H).

### Step 5: N-((4-((2-cyclopropyl-6-isopropyl-7-oxopyrazolo[1,5-a]pyrimidin-4(7H)-yl)methyl)phenyl)(methyl)(oxo)-l6-sulfaneylidene)-4-methylbenzenesulfonamide

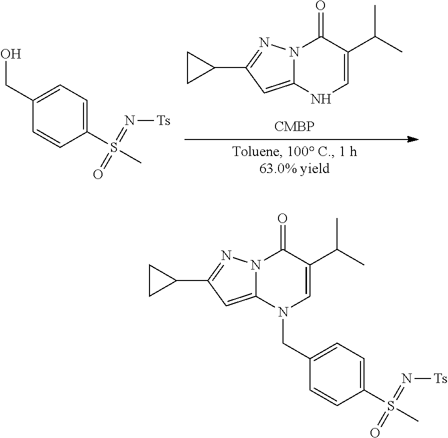 [SMILES: Cc1ccc(S(=O)(=O)N=S(C)(=O)c2ccc(CO)cc2)cc1 ; CC(C)c1c[nH]c2cc(C3CC3)nn2c1=O ; Cc1ccc(S(=O)(=O)N=S(C)(=O)c2ccc(Cn3cc(C(C)C)c(=O)n4nc(C5CC5)cc34)cc2)cc1]

To a stirred mixture of [4-(hydroxymethyl)phenyl](methyl) [(4-methylbenzenesulfonyl)imino]-lambda6-sulfanone (600 mg, 1.76 mmol, 1.0 equiv, enantiomeric ratio: 2:8) and 2-cyclopropyl-6-isopropyl-4H-pyrazolo[1,5-a]pyrimidin-7-one (460 mg, 2.12 mmol, 1.2 equiv) in toluene (6 mL) was added CMBP (1.28 g, 5.30 mmol, 3.0 equiv) at 25° C. under nitrogen atmosphere. The resulting mixture was stirred for 1 h at 100° C. under nitrogen atmosphere. The mixture was allowed to cool down to 25° C. The desired product could be detected by LCMS. The resulting mixture was concentrated under reduced pressure. The residue was purified by reversed-phase flash chromatography with the following conditions: column: C18; mobile phase: MeCN in Water (0.1% FA), 15% to 45% gradient in 20 min; detector: UV 220 nm, the pure fraction was concentrated under reduced pressure to afford N-((4-((2-cyclopropyl-6-isopropyl-7-oxopyrazolo[1,5-a]pyrimidin-4(7H)-yl)methyl)phenyl)(methyl)(oxo)-l6-sulfaneylidene)-4-methylbenzenesulfonamide.

ESI-MS m/z=539.00 [M+H]⁺; Calculated: 538.2.

### Step 6: 2-Cyclopropyl-6-isopropyl-4-(4-(S-methylsulfonimidoyl)benzyl)pyrazolo[1,5-a]pyrimidin-7(4H)-one

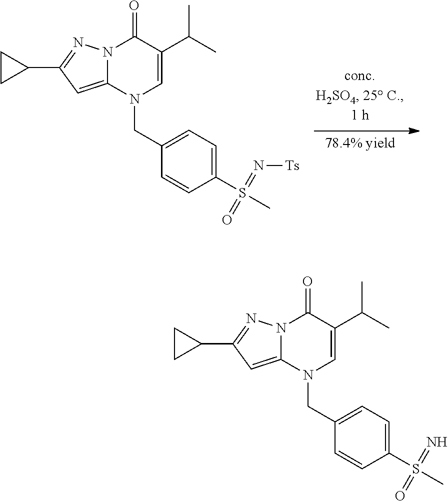 [SMILES: Cc1ccc(S(=O)(=O)N=S(C)(=O)c2ccc(Cn3cc(C(C)C)c(=O)n4nc(C5CC5)cc34)cc2)cc1 ; CC(C)c1cn(Cc2ccc(S(C)(=N)=O)cc2)c2cc(C3CC3)nn2c1=O]

A solution of N-((4-((2-cyclopropyl-6-isopropyl-7-oxopyrazolo[1,5-a]pyrimidin-4(7H)-yl)methyl)phenyl)(methyl)(oxo)-l6-sulfaneylidene)-4-methylbenzenesulfonamide (500 mg, 0.92 mmol, 1.0 equiv) in H₂SO₄ (5 mL) was stirred for 1 h at 25° C. under nitrogen atmosphere. The desired product could be detected by LCMS. Dropped the resulting mixture into the water (10 mL) at 0° C. The mixture was basified to pH 9 with NaOH(aq.). The aqueous layer was extracted with CHCl3/i-PrOH (10:1, 3×20 mL). The combined organic layers were dried over anhydrous Na₂SO₄. After filtration, the filtrate was concentrated under reduced pressure. The residue was purified by reversed-phase flash chromatography with the following conditions: column: C18; mobile phase: MeCN in Water (10 mmol/L NH₄HCO₃), 15% to 45% gradient in 15 min; detector: UV 220 nm, the pure fraction was concentrated under reduced pressure to afford 2-cyclopropyl-6-isopropyl-4-(4-(S-methylsulfonimidoyl)benzyl)pyrazolo[1,5-a]pyrimidin-7(4H)-one.

ESI-MS m/z=385.20 [M+H]⁺; Calculated: 384.2.

¹H NMR (400 MHZ, DMSO-d₆) δ 7.96 (s, 1H), 7.94-7.88 (m, 2H), 7.56-7.50 (m, 2H), 6.03 (s, 1H), 5.32 (s, 2H), 4.21 (s, 1H), 3.07-2.93 (m, 4H), 1.99-1.88 (m, 1H), 1.20 (d, J=6.9 Hz, 3H), 1.20 (d, J=6.9 Hz, 3H), 0.98-0.87 (m, 2H), 0.78-0.70 (m, 2H).

### Step 7: Rel-(R)-2-cyclopropyl-6-isopropyl-4-(4-(S-methylsulfonimidoyl)benzyl)pyrazolo[1,5-a]pyrimidin-7(4H)-one

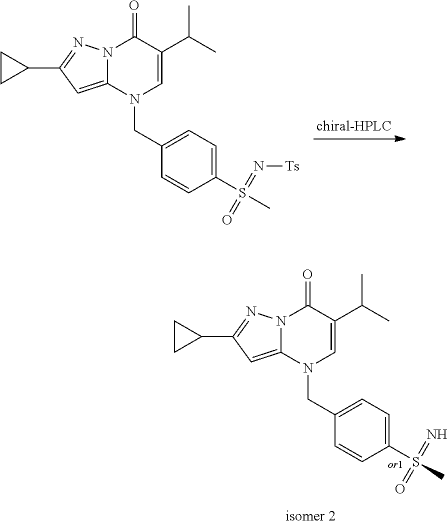 [SMILES: Cc1ccc(S(=O)(=O)N=S(C)(=O)c2ccc(Cn3cc(C(C)C)c(=O)n4nc(C5CC5)cc34)cc2)cc1 ; CC(C)c1cn(Cc2ccc([S@](C)(=N)=O)cc2)c2cc(C3CC3)nn2c1=O]

The racemate (280 mg) was separated by Chiral-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 212/240 nm; RT1 (min): 17.1; Sample Solvent: HFIP: DCM=1:1; Number Of Runs: 5), the pure fraction was concentrated and then lyophilized respectively to afford rel-(R)-2-cyclopropyl-6-isopropyl-4-(4-(S-methylsulfonimidoyl)-benzyl) pyrazolo[1,5-a]pyrimidin-7(4H)-one.

ESI-MS m/z=385.05 [M+H]⁺; Calculated: 384.2.

¹H NMR (400 MHZ, DMSO-d₆) δ 7.97-7.87 (m, 3H), 7.56-7.49 (m, 2H), 6.02 (s, 1H), 5.32 (s, 2H), 4.21 (s, 1H), 3.06-2.95 (m, 4H), 1.99-1.87 (m, 1H), 1.20 (d, J=6.9 Hz, 3H), 1.20 (d, J=6.9 Hz, 3H), 0.98-0.87 (m, 2H), 0.78-0.70 (m, 2H).

Total proton count from structure: 24.

Total proton count from spectrum: 24.

### Compound 3B: rel-(S)-2-cyclopropyl-6-isopropyl-4-(4-(S-methylsulfonimidoyl)benzyl)pyrazolo[1,5-a]pyrimidin-7(4H)-one

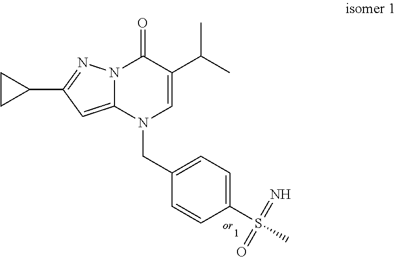 [SMILES: CC(C)c1cn(Cc2ccc([S@@](C)(=N)=O)cc2)c2cc(C3CC3)nn2c1=O]

### Step 1: Rel-(S)-2-cyclopropyl-6-isopropyl-4-(4-(S-methylsulfonimidoyl)benzyl)pyrazolo[1,5-a]pyrimidin-7(4H)-one

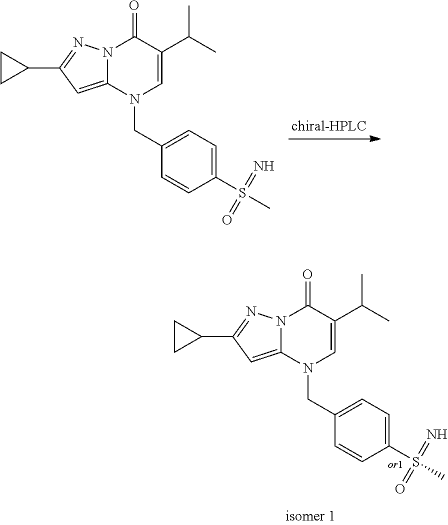 [SMILES: CC(C)c1cn(Cc2ccc(S(C)(=N)=O)cc2)c2cc(C3CC3)nn2c1=O ; CC(C)c1cn(Cc2ccc([S@@](C)(=N)=O)cc2)c2cc(C3CC3)nn2c1=O]

The racemate (280 mg) was separated by Chiral-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 212/240 nm; RT2 (min): 23.5; Sample Solvent: HFIP: DCM=1:1; Number Of Runs: 5), the pure fraction was concentrated and then lyophilized respectively to afford Rel-(S)-2-cyclopropyl-6-isopropyl-4-(4-(S-methylsulfonimidoyl)benzyl)-pyrazolo[1,5-a]pyrimidin-7(4H)-one.

ESI-MS m/z=385.30 [M+H]⁺; Calculated: 384.2.

¹H NMR (400 MHZ, DMSO-d₆) δ 7.98-7.88 (m, 3H), 7.56-7.49 (m, 2H), 6.03 (s, 1H), 5.32 (s, 2H), 4.21 (s, 1H), 3.07-2.95 (m, 4H), 1.99-1.88 (m, 1H), 1.20 (d, J=6.9 Hz, 3H), 1.20 (d, J=6.9 Hz, 3H), 0.98-0.89 (m, 2H), 0.78-0.70 (m, 2H).

Total proton count from structure: 24.

Total proton count from spectrum: 24.

### Compound 4: (1S,4S,5S)-5-((3-chloro-6-isopropyl-2-methyl-7-oxopyrazolo[1,5-a]pyrimidin-4(7H)-yl)methyl)-2-azabicyclo[2.2.1]heptane-2-sulfonamide

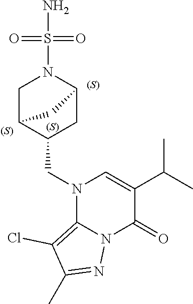

### Step 1: 3-Chloro-6-isopropyl-2-methylpyrazolo[1,5-a]pyrimidin-7(4H)-one

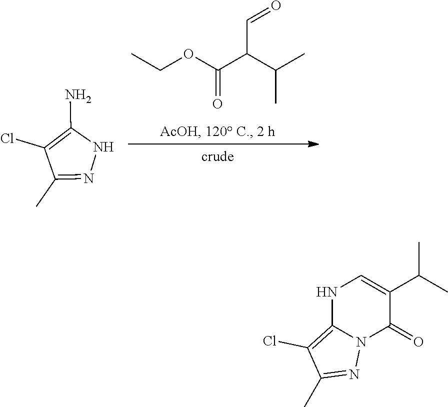

A solution of 4-chloro-5-methyl-2H-pyrazol-3-amine (500 mg, 3.80 mmol, 1.0 equiv), ethyl 2-formyl-3-methylbutanoate (661 mg, 4.18 mmol, 1.1 equiv) in AcOH (4 mL) was stirred for 2 h at 120° C. under nitrogen atmosphere. The reaction was monitored by LCMS. The mixture was allowed to cool down to 25° C. The product was precipitated by the addition of water. The precipitated solids were collected by filtration and washed with water (2×20 mL). This resulted in 3-chloro-6-isopropyl-2-methylpyrazolo[1,5-a]pyrimidin-7(4H)-one (crude).

ESI-MS m/z=226.1 [M+H]⁺; Calculated: 225.1.

¹H NMR (400 MHZ, DMSO-d₆) δ 12.41 (s, 1H), 7.56 (s, 1H), 3.08-2.91 (m, 1H), 2.27 (d, J=1.3 Hz, 3H), 1.18 (d, J=6.9 Hz, 6H).

### Step 2: Tert-butyl(1S,4S,5S)-5-((3-chloro-6-isopropyl-2-methyl-7-oxopyrazolo[1,5-a]pyrimidin-4(7H)-yl)methyl)-2-azabicyclo[2.2.1]heptane-2-carboxylate

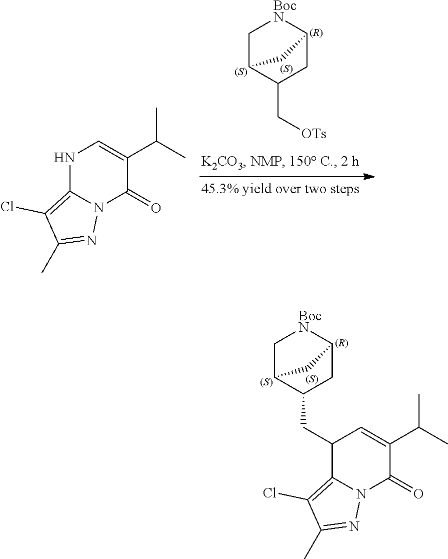

A solution of tert-butyl(1R,4S,5S)-5-{[(4-methylbenzenesulfonyl)oxy]methyl}-2-azabicyclo[2.2.1]heptane-2-carboxylate (370 mg, 0.97 mmol, 1.1 equiv), {6-isopropyl-2-methyl-7-oxo-4H-pyrazolo[1,5a]pyrimidin-3-yl}chloranium (200 mg, 0.88 mmol, 1.0 equiv) and K₂CO₃ (365 mg, 2.64 mmol, 3.0 equiv) in 1-methylpyrrolidin-2-one (5 mL) was stirred for 2 h at 150° C. under nitrogen atmosphere. The desired product could be detected by LCMS. The mixture was allowed to cool down to 25° C. The resulting mixture was diluted with EtOAc (10 mL). The organic layers were washed with water (5×10 mL), and dried over anhydrous Na₂SO₄. After filtration, the filtrate was concentrated under reduced pressure. The residue was purified by silica gel column chromatography, eluted with PE/EA (10:1-5:1) to afford tert-butyl(1S,4S,5S)-5-((3-chloro-6-isopropyl-2-methyl-7-oxopyrazolo[1,5-a]pyrimidin-4(7H)-yl)methyl)-2-azabicyclo[2.2.1]heptane-2-carboxylate.

ESI-MS m/z=435.0 [M+H]⁺; Calculated: 434.2.

### Step 3: 4-(((1R,4S,5S)-2-Azabicyclo[2.2.1]heptan-5-yl)methyl)-3-chloro-6-isopropyl-2-methylpyrazolo[1,5-a]pyrimidin-7(4H)-one hydrochloride

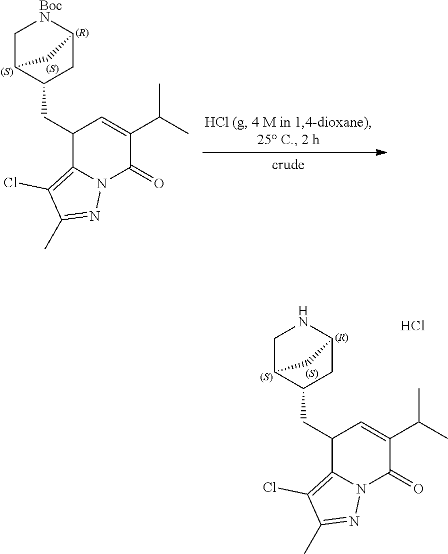

A solution of tert-butyl(1S,4S,5S)-5-({3-chloro-6-isopropyl-2-methyl-7-oxopyrazolo[1,5-a]pyrimidin-4-yl}methyl)-2-azabicyclo[2.2.1]heptane-2-carboxylate (200 mg, 0.46 mmol, 1.0 equiv) in HCl (g, 4 M in 1,4-dioxane, 4 mL) was stirred for 2 h at 25° C. under nitrogen atmosphere. The mixture was allowed to cool down to 25° C. The desired product could be detected by LCMS. The resulting mixture was concentrated under reduced pressure. The residue was purified by trituration with Et₂O (3×10 mL). This resulted in 4-(((1R,4S,5S)-2-azabicyclo[2.2.1]heptan-5-yl)methyl)-3-chloro-6-isopropyl-2-methylpyrazolo[1,5-a]pyrimidin-7(4H)-one hydrochloride (crude).

ESI-MS m/z=335.2 [M-HCl+H]⁺; Calculated: 370.1.

### Step 4: (1S,4S,5S)-5-((3-Chloro-6-isopropyl-2-methyl-7-oxopyrazolo[1,5-a]pyrimidin-4(7H)-yl)methyl)-2-azabicyclo[2.2.1]heptane-2-sulfonamide

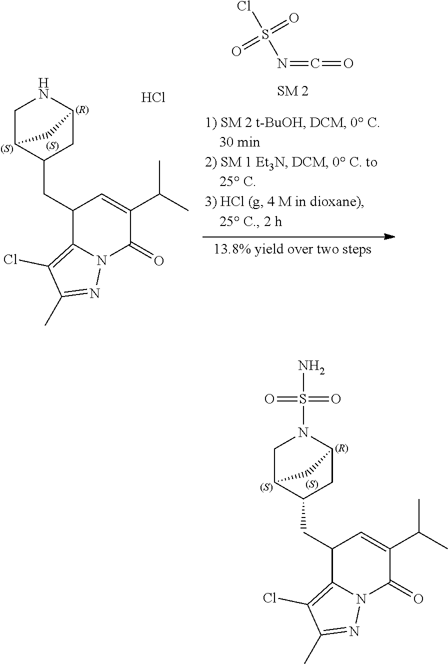

A mixture of chlorosulfonyl isocyanate (457 mg, 3.23 mmol, 6.0 equiv) in DCM (2 mL) was added t-BuOH (359 mg, 4.85 mmol, 9.0 equiv) at 0° C. under nitrogen atmosphere. A solution of 4-[(1R,4S,5S)-2-azabicyclo[2.2.1]heptan-5-ylmethyl]-3-chloro-6-isopropyl-2-methylpyrazolo[1,5-a]pyrimidin-7-one hydrochloride (200 mg, 0.53 mmol, 1.0 equiv) in DCM (2 mL) was treated with triethylamine (545 mg, 5.39 mmol, 10.0 equiv) for 5 min at 25° C. under nitrogen atmosphere followed by the addition of above mixture dropwise at 0° C. The resulting mixture was stirred for 2 h at 25° C. under nitrogen atmosphere. The reaction was monitored by LCMS. To the above mixture was added HCl (g, 4 M in 1,4-dioxane, 2 mL) at 25° C. The resulting mixture was stirred 2 h at 25° C. The reaction was monitored by LCMS. The resulting mixture was concentrated under reduced pressure. The residue was dissolved in water (20 mL). The resulting mixture was extracted with EtOAc (2×20 mL). The combined organic layers were washed with brine (1×20 mL), and dried over anhydrous Na₂SO₄. After filtration, the filtrate was concentrated under reduced pressure. The residue was purified by Prep-HPLC with the following conditions: (Column: YMC-Actus Triart C18 ExRS 30*150 mm, 5 m; Mobile Phase A: Water (10 mmol/L NH₄HCO₃), Mobile Phase B: ACN; Flow rate: 60 mL/min mL/min; Gradient: 10% B to 10% B in 1.5 min, 10% B to 10% B in 2 min, 20% B to 40% B in 10 min; Wave Length: 254 nm/220 nm; RT1 (min): 10.12), the pure fraction was concentrated then lyophilized to afford (1S,4S,5S)-5-((3-chloro-6-isopropyl-2-methyl-7-oxopyrazolo[1,5-a]pyrimidin-4(7H)-yl)methyl)-2-azabicyclo[2.2.1]heptane-2-sulfonamide.

ESI-MS m/z=414.05 [M+H]⁺; Calculated: 413.1.

¹H NMR (400 MHZ, DMSO-d₆) δ 7.78 (s, 1H), 6.67 (s, 2H), 4.24-4.08 (m, 2H), 3.95 (s, 1H), 3.09 (dd, J=9.1, 3.5 Hz, 1H), 3.04-2.94 (m, 1H), 2.72 (d, J=9.1 Hz, 1H), 2.36-2.25 (m, 4H), 2.24-2.15 (m, 1H), 1.96-1.84 (m, 1H), 1.73-1.57 (m, 2H), 1.31-1.24 (m, 1H), 1.18 (dd, J=6.9, 2.1 Hz, 6H).

Total proton count from structure: 24.

Total proton count from spectrum: 24.

### Compound 5: 4-((2-cyclopropyl-6-isopropyl-7-oxo-[1,2,4]triazolo[1,5-a]pyrimidin-4(7H)-yl)methyl)-2-(pyridin-4-yl)benzenesulfonamide

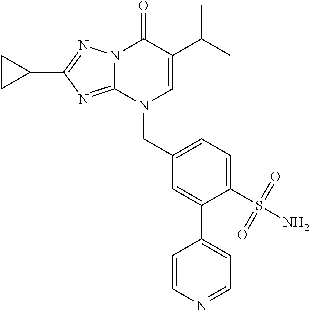 [SMILES: CC(C)c1cn(Cc2ccc(S(N)(=O)=O)c(-c3ccncc3)c2)c2nc(C3CC3)nn2c1=O]

### Step 1: 4-Cyano-2-(pyridin-4-yl)benzenesulfonamide

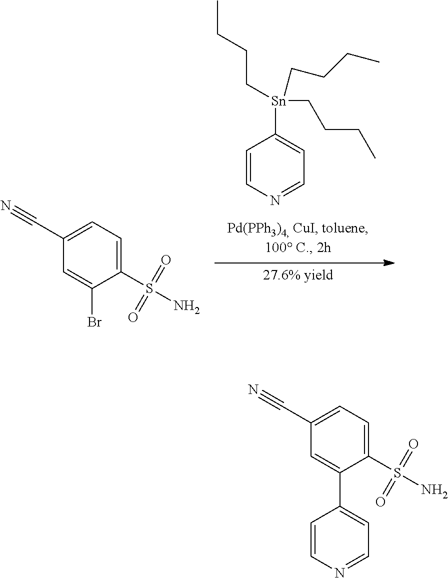 [SMILES: CCC[CH2][Sn]([CH2]CCC)([CH2]CCC)[c]1ccncc1 ; N#Cc1ccc(S(N)(=O)=O)c(Br)c1 ; N#Cc1ccc(S(N)(=O)=O)c(-c2ccncc2)c1]

To a stirred solution of 2-bromo-4-cyanobenzenesulfonamide (4.00 g, 15.3 mmol, 1.0 equiv) and 4-(tributylstannyl)pyridine (5.64 g, 15.3 mmol, 1.0 equiv) in Toluene (40 mL) were added Pd(PPh₃)₄ (0.37 g, 1.53 mmol, 0.1 equiv) and CuI (2.92 g, 15.3 mmol, 1.0 equiv) at 25° C. under nitrogen atmosphere. The resulting mixture was stirred for an additional 2 h at 100° C. The desired product could be detected by LCMS. The mixture was allowed to cool down to 25° C. The resulting mixture was diluted with water (100 mL). The resulting mixture was extracted with EtOAc (3×150 mL). The combined organic layers were washed with brine (100 mL), and dried over anhydrous Na₂SO₄. After filtration, the filtrate was concentrated under reduced pressure. The residue was purified by silica gel column chromatography, eluted with PE/EA (1:1) to afford in 4-cyano-2-(pyridin-4-yl)benzenesulfonamide.

ESI-MS m/z=260.0 [M+H]⁺; Calculated: 259.0.

### Step 2: 4-Formyl-2-(pyridin-4-yl)benzenesulfonamide

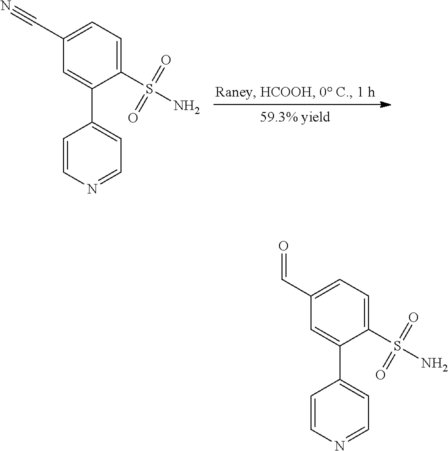 [SMILES: N#Cc1ccc(S(N)(=O)=O)c(-c2ccncc2)c1 ; NS(=O)(=O)c1ccc(C=O)cc1-c1ccncc1]

To a stirred solution of 4-cyano-2-(pyridin-4-yl)benzenesulfonamide (200 mg, 0.77 mmol, 1.0 equiv) in HCOOH (2 mL) were added Raney-Ni (0.66 mg, 0.01 mmol, 0.01 equiv, 10% wt) at 25° C. under nitrogen atmosphere. The resulting mixture was stirred for an additional 1 h at 0° C. The desired product could be detected by LCMS. The resulting mixture was filtered, and the filter cake was washed with CHCl3: IPA (5:1) (3×100 mL). The filtrate was concentrated under reduced pressure. The residue was purified by reversed-phase flash chromatography with the following conditions: column: C18; mobile phase: MeCN in Water (10 mmol/L NH₄HCO₃), 20% to 50% gradient in 20 min; detector: UV 254 nm. This resulted in 4-formyl-2-(pyridin-4-yl)benzenesulfonamide.

ESI-MS m/z=263.0 [M+H]⁺; Calculated: 262.0.

¹H NMR (300 MHZ, DMSO-d₆) δ 8.63-8.54 (m, 2H), 8.02 (d, J=8.2 Hz, 1H), 7.72-7.66 (m, 2H), 7.63 (dd, J=8.3, 1.7 Hz, 1H), 7.32 (d, J=1.7 Hz, 1H), 6.05 (s, 1H), 4.61 (s, 2H).

### Step 3: 4-(Hydroxymethyl)-2-(pyridin-4-yl)benzenesulfonamide

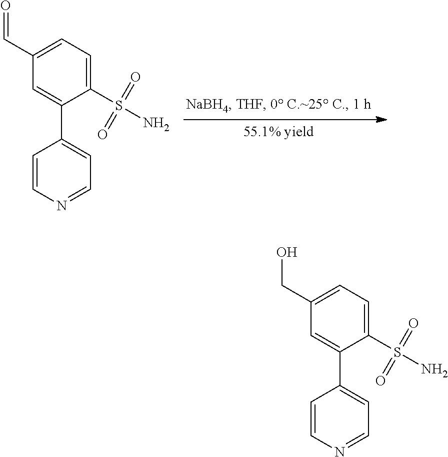 [SMILES: NS(=O)(=O)c1ccc(C=O)cc1-c1ccncc1 ; NS(=O)(=O)c1ccc(CO)cc1-c1ccncc1]

To a stirred solution of 4-formyl-2-(pyridin-4-yl)benzenesulfonamide (180 mg, 0.686 mmol, 1.0 equiv) in THF (2 mL) were added NaBH₄ (52 mg, 1.37 mmol, 2.0 equiv) at 0° C. The resulting mixture was stirred for an additional 1 h at 25° C. The desired product could be detected by LCMS. The reaction was quenched with Water/Ice at 0° C. The resulting mixture was concentrated under reduced pressure. The residue was purified by reversed-phase flash chromatography with the following conditions: column: C18; mobile phase: MeCN in Water (0.1% FA), 30% to 50% gradient in 20 min; detector: UV 254 nm. This resulted in 4-(hydroxymethyl)-2-(pyridin-4-yl)benzenesulfonamide.

ESI-MS m/z=265.1 [M+H]⁺; Calculated: 264.1.

### Step 4: 4-((2-Cyclopropyl-6-isopropyl-7-oxo-[1,2,4]triazolo[1,5-a]pyrimidin-4(7H)-yl)methyl)-2-(pyridin-4-yl)benzenesulfonamide

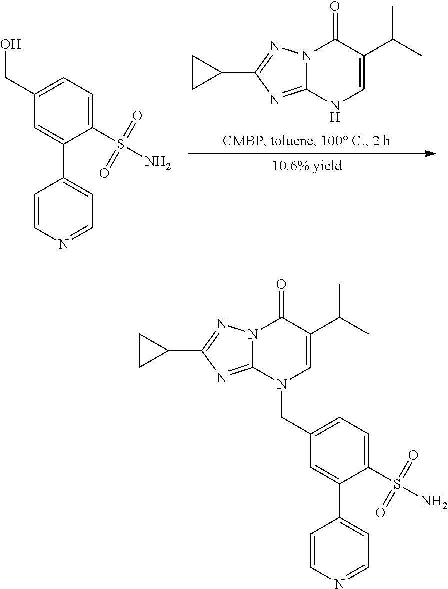 [SMILES: NS(=O)(=O)c1ccc(CO)cc1-c1ccncc1 ; CC(C)c1c[nH]c2nc(C3CC3)nn2c1=O ; CC(C)c1cn(Cc2ccc(S(N)(=O)=O)c(-c3ccncc3)c2)c2nc(C3CC3)nn2c1=O]

To a stirred solution of 4-(hydroxymethyl)-2-(pyridin-4-yl)benzenesulfonamide (50 mg, 0.19 mmol, 1.0 equiv) and 2-cyclopropyl-6-isopropyl-4H-[1,2,4]triazolo[1,5-a]pyrimidin-7-one (40 mg, 0.19 mmol, 1.0 equiv) in Toluene (2 mL) were added CMBP (457 mg, 1.89 mmol, 10.0 equiv) at 25° C. under nitrogen atmosphere. The resulting mixture was stirred for an additional 2 h at 100° C. under nitrogen atmosphere. The desired product could be detected by LCMS. The mixture was allowed to cool down to 25° C. The residue was purified by silica gel column chromatography, eluted with PE/EA (1:1) to afford crude product. The crude product (70 mg) was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min mL/min; Gradient: 20% B to 30% B in 10 min; Wave Length: 254 nm/220 nm nm; RT1 (min): 9.1) the pure fraction was concentrated then lyophilized to afford 4-(2-cyclopropyl-6-isopropyl-7-oxo-[1,2,4]triazolo[1,5-a]pyrimidin-4(7H)-yl)methyl)-2-(pyridin-4-yl)benzenesulfonamide.

ESI-MS m/z=465.15 [M+H]⁺; Calculated: 464.2.

¹H NMR (400 MHZ, DMSO-d₆) δ 8.65-8.56 (m, 2H), 8.04 (s, 1H), 8.00 (d, J=8.2 Hz, 1H), 7.59 (dd, J=8.3, 1.9 Hz, 1H), 7.48-7.42 (m, 3H), 7.39-7.34 (m, 2H), 5.39 (s, 2H), 2.98-2.90 (m, 1H), 2.09-1.95 (m, 1H), 1.18 (d, J=6.9 Hz, 6H), 0.92 (m, 4H).

Total proton count from structure: 24

Total proton count from spectrum: 24

### Compound 6: (1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-sulfonamide

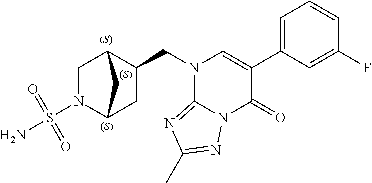

### Step 1: Methyl 2-(3-fluorophenyl)-3-oxopropanoate

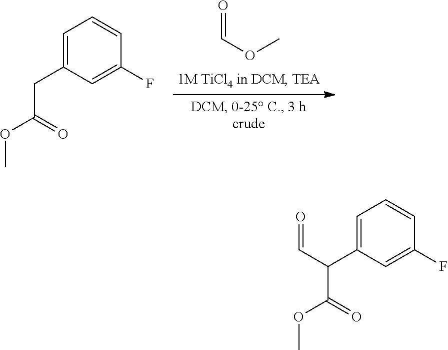

To a stirred solution of methyl 2-(3-fluorophenyl)acetate (20.00 g, 118.9 mmol, 1.0 equiv) and methyl formate (21.43 g, 356.8 mmol, 3.0 equiv) in THF (400 mL) were added 1M TiCl4 in DCM (238 mL, 237.9 mmol, 2.0 equiv) and TEA (28.88 g, 285.4 mmol, 2.4 equiv) dropwise at 0° C. under nitrogen atmosphere. The resulting mixture was stirred for 3 h at 25° C. under nitrogen atmosphere. The reaction was monitored by LCMS. The reaction was then quenched by the addition of 500 ml of water. The resulting mixture was extracted with EtOAc (3×500 mL). The combined organic layers were washed with brine (1×1500 mL), and dried over anhydrous Na₂SO₄. After filtration, the filtrate was concentrated under reduced pressure. This resulted in methyl 2-(3-fluorophenyl)-3-oxopropanoate (crude).

ESI-MS m/z=194.95 [M−H]⁻; Calculated: 196.1.

### Step 2: 6-(3-Fluorophenyl)-2-methyl-[1,2,4]triazolo[1,5-a]pyrimidin-7(4H)-one

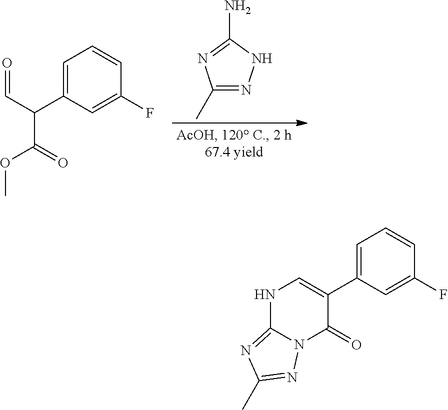

To a stirred mixture of 5-methyl-2H-1,2,4-triazol-3-amine (2.10 g, 21.4 mmol, 1.0 equiv) in AcOH (42 mL) was added methyl 2-(3-fluorophenyl)-3-oxopropanoate (5.04 g, 25.7 mmol, 1.2 equiv) at 25° C. The mixture was stirred for 2 h at 120° C. under nitrogen atmosphere. The desired product could be detected by LCMS. The mixture was allowed to cool down to 25° C. The resulting mixture was poured into water (2 L) then stirred for 30 min. The precipitated solids were collected by filtration and washed with water (3×100 mL) then concentrated under reduced pressure. The residue was purified by trituration with PE/EA (10:1, 800 mL). The precipitated solids were collected by filtration and washed with PE (3×100 mL). The solids were concentrated under vacuum to afford 6-(3-fluorophenyl)-2-methyl-[1,2,4]triazolo[1,5-a]pyrimidin-7(4H)-one.

ESI-MS m/z=245.10 [M+H]⁺; Calculated: 244.1.

¹H NMR (400 MHZ, DMSO-d₆) δ 13.54 (s, 1H), 8.28 (s, 1H), 7.62-7.52 (m, 2H), 7.51-7.40 (m, 1H), 7.23-7.11 (m, 1H), 2.40 (s, 3H).

¹⁹F NMR (377 MHz, DMSO-d₆) δ −113.54.

### Step 3: Tert-butyl(1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-carboxylate

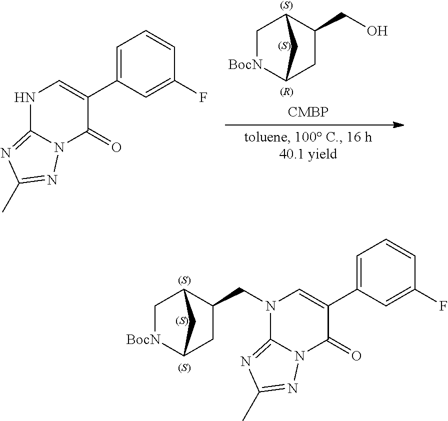

To a stirred mixture of 6-(3-fluorophenyl)-2-methyl-[1,2,4]triazolo[1,5-a]pyrimidin-7(4H)-one (451 mg, 1.85 mmol, 1.2 equiv) and tert-butyl(1R,4S,5S)-5-(hydroxymethyl)-2-azabicyclo[2.2.1]heptane-2-carboxylate (350 mg, 1.54 mmol, 1.0 equiv) in toluene (7 mL) was added 2-(tributyl-lambda5-phosphanylidene) acetonitrile (1.86 g, 7.70 mmol, 5.0 equiv) at 25° C. The resulting mixture was stirred for 16 h at 100° C. under nitrogen atmosphere. The desired product could be detected by LCMS. The resulting mixture was diluted with water (200 mL). The resulting mixture was extracted with EA (3×200 mL). The combined organic layers were washed with brine (1×200 mL), and dried over anhydrous Na₂SO₄. After filtration, the filtrate was concentrated under reduced pressure. The residue was purified by silica gel column chromatography, eluted with MeOH in DCM (0% to 10% gradient in 20 min, hold at 3%) to afford the crude product. The crude product was purified by reverse phase flash with the following conditions (column: C18; mobile phase: MeCN in Water (0.1% FA), 30% to 80% gradient in 20 min, hold at 38%; detector: UV 254 nm), the pure factions were extracted with EA (3×200 mL). The combined organic layers were washed with brine (2×200 mL), and dried over anhydrous Na₂SO₄. After filtration, the filtrate was concentrated under reduced pressure to afford tert-butyl(1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-carboxylate.

ESI-MS m/z=454.20 [M+H]⁺; Calculated: 453.2.

¹H NMR (400 MHZ, DMSO-d₆) δ 8.59 (s, 1H), 7.62-7.53 (m, 2H), 7.52-7.44 (m, 1H), 7.24-7.14 (m, 1H), 4.27-4.16 (m, 1H), 4.10-3.99 (m, 2H), 3.15-3.05 (m, 1H), 2.84 (d, J=9.7 Hz, 1H), 2.47-2.30 (m, 5H), 1.80-1.64 (m, 2H), 1.58-1.47 (m, 1H), 1.44-1.29 (m, 10H).

¹⁹F NMR (377 MHZ, DMSO-d₆) δ −113.49.

### Step 4: 4-(((1R,4S,5S)-2-azabicyclo[2.2.1]heptan-5-yl)methyl)-6-(3-fluorophenyl)-2-methyl-[1,2,4]triazolo[1,5-a]pyrimidin-7(4H)-one HCl salt

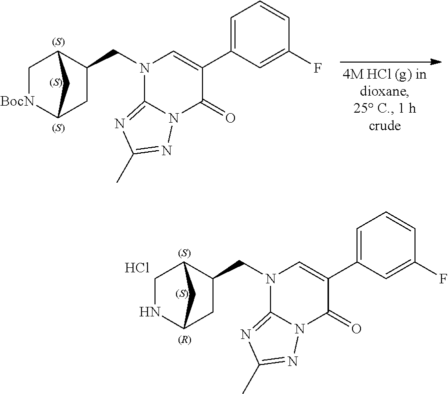

A mixture of tert-butyl(1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-carboxylate (100 mg, 0.22 mmol, 1.0 equiv) in 4M HCl (g) in 1,4-dioxane (2 mL) was stirred for 1 h at 25° C. under nitrogen atmosphere. The desired product could be detected by LCMS. The resulting mixture was concentrated under reduced pressure to afford 4-(((1R,4S,5S)-2-azabicyclo[2.2.1]heptan-5-yl)methyl)-6-(3-fluorophenyl)-2-methyl-[1,2,4]triazolo[1,5-a]pyrimidin-7(4H)-one HCl salt (crude).

ESI-MS m/z=354.15 [M−HCl+H]⁺; Calculated: 389.1.

### Step 5: (1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-sulfonamide

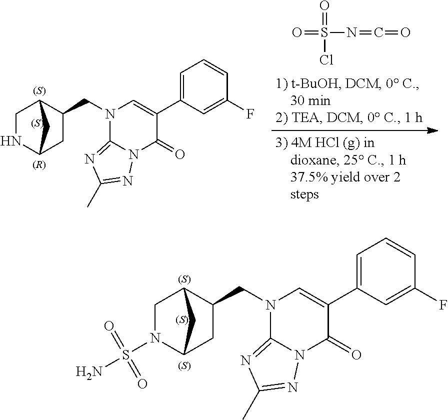

To a stirred mixture of chlorosulfonyl isocyanate (187 mg, 1.32 mmol, 6.0 equiv) in DCM (1.2 mL) was added t-BuOH (147 mg, 1.98 mmol, 9.0 equiv) dropwise at 0° C. under nitrogen atmosphere. The resulting mixture was stirred for 30 min at 0° C. under nitrogen atmosphere. A stirred mixture of 4-(((1R,4S,5S)-2-azabicyclo[2.2.1]heptan-5-yl)methyl)-6-(3-fluorophenyl)-2-methyl-[1,2,4]triazolo[1,5-a]pyrimidin-7(4H)-one HCl salt (120 mg, 0.22 mmol, 1.0 equiv) in DCM (5 mL) was treated with TEA (223 mg, 2.20 mmol, 10.0 equiv) for 5 min at 25° C. under nitrogen atmosphere followed by the addition of above mixture dropwise at 0° C. The resulting mixture was stirred for 1 h at 0° C. under nitrogen atmosphere. The reaction was monitored by LCMS. The resulting mixture was concentrated under reduced pressure. To the above mixture was added 4M HCl (g) in 1,4-dioxane (5 mL) at 25° C. The resulting mixture was stirred for 1 h at 25° C. under nitrogen atmosphere. The resulting mixture was concentrated under reduced pressure. The resulting mixture was diluted with water (50 mL). The mixture was basified to pH 8 with saturated Na₂CO₃ (aq.). The resulting mixture was extracted with EA (3×50 mL). The combined organic layers were washed with brine (2×50 mL), and dried over anhydrous Na₂SO₄. After filtration, the filtrate was concentrated under reduced pressure. The crude product (120 mg) was purified by Prep-HPLC with the following conditions (Column: XBridge Prep Phenyl OBD Column 19*250 mm, 5 μm; Mobile Phase A: 10 mmol/L NH₄HCO₃+0.05% NH₃H₂O), Mobile Phase B: ACN; Flow rate: 60 mL/min; Gradient: 5% B to 5% B in 1 min, 5% B to 15% B in 2 min, 15% to 35% B in 10 min; Wave Length: 254 nm/220 nm; RT1 (min): 9.42), the pure factions were concentrated under vacuum then lyophilized to afford (1S,4S,5S)-5-((6-(3-fluorophenyl)-2-methyl-7-oxo-[1,2,4]triazolo[1,5-a]pyrimidin-4(7H)-yl)methyl)-2-azabicyclo[2.2.1]heptane-2-sulfonamide.

ESI-MS m/z=433.10 [M+H]⁺; Calculated: 432.1.

¹H NMR (400 MHZ, DMSO-d₆) δ 8.53 (s, 1H), 7.60-7.45 (m, 3H), 7.25-7.16 (m, 1H), 6.68 (s, 2H), 4.16 (dd, J=13.8, 9.1 Hz, 1H), 4.02 (dd, J=13.8, 6.6 Hz, 1H), 3.98-3.92 (m, 1H), 3.09 (dd, J=9.1, 3.6 Hz, 1H), 2.74 (d, J=9.1 Hz, 1H), 2.47-2.37 (m, 5H), 1.96-1.86 (m, 1H), 1.69 (d, J=10.2 Hz, 1H), 1.62 (d, J=10.3 Hz, 1H), 1.36-1.27 (m, 1H).

¹⁹F NMR (377 MHZ, DMSO-d₆) δ 113.454.

Total proton count from structure: 21.

Total proton count from spectrum: 21.

### Compound 7A: Rel-(R)-2-cyclopropyl-6-isopropyl-4-(4-(S-methylsulfonimidoyl)benzyl)-[1,2,4]triazolo[1,5-a]pyrimidin-7(4H)-one

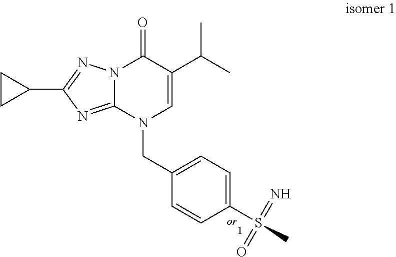 [SMILES: CC(C)c1cn(Cc2ccc([S@](C)(=N)=O)cc2)c2nc(C3CC3)nn2c1=O]

### Step 1: 2-Cyclopropyl-6-isopropyl-4-(4-(S-methylsulfonimidoyl)benzyl)-[1,2,4]triazolo[1,5-a]pyrimidin-7(4H)-one

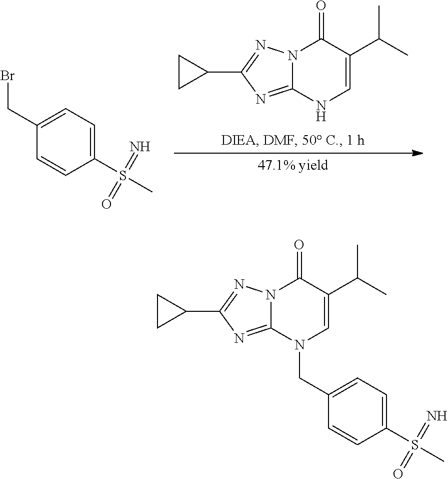 [SMILES: CS(=N)(=O)c1ccc(CBr)cc1 ; CC(C)c1c[nH]c2nc(C3CC3)nn2c1=O ; CC(C)c1cn(Cc2ccc(S(C)(=N)=O)cc2)c2nc(C3CC3)nn2c1=O]

A solution of [4-(bromomethyl)phenyl](imino)methyl-lambda6-sulfanone (8.19 g, 32.9 mmol, 1.2 equiv) and 2-cyclopropyl-6-isopropyl-4H-[1,2,4]triazolo[1,5-a]pyrimidin-7-one (6.00 g, 27.4 mmol, 1.0 equiv), DIEA (10.66 g, 82.4 mmol, 3.0 equiv) in DMF (60 mL) was stirred for 2 h at 50° C. under nitrogen atmosphere. The reaction was monitored by LCMS. The mixture was allowed to cool down to room temperature. The resulting mixture was diluted with EtOAc (500 mL). The resulting mixture was washed with 3×500 ml of water. The aqueous layer was extracted with EtOAc (3×500 mL). The resulting mixture was concentrated under vacuum. The crude product was purified by HP-flash with the following conditions (Column: YMC-Triart Prep C18-S column 100*250 mm, S-100 μm; Mobile Phase A: Water (10 mmol/L NH₄HCO₃), Mobile Phase B: MEOH; Flow rate: 140 mL/min mL/min; Gradient: 20% B to 50% B in 45 min; Wave Length: 254 nm/220 nm nm; RT1 (min): 20), to afford 2-cyclopropyl-4-({4-[imino(methyl)oxo-lambda6-sulfanyl]phenyl}methyl)-6-isopropyl-[1,2,4]triazolo[1,5-a]pyrimidin-7-one.

ESI-MS m/z=386.10 [M+H]⁺; Calculated: 385.2.

### Step 2: Rel-(R)-2-cyclopropyl-6-isopropyl-4-(4-(S-methylsulfonimidoyl)benzyl)-[1,2,4]triazolo[1,5-a]pyrimidin-7(4H)-one

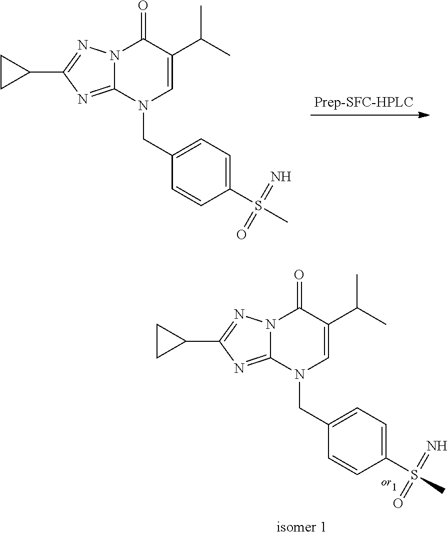 [SMILES: CC(C)c1cn(Cc2ccc(S(C)(=N)=O)cc2)c2nc(C3CC3)nn2c1=O ; CC(C)c1cn(Cc2ccc([S@](C)(=N)=O)cc2)c2nc(C3CC3)nn2c1=O]

The racemate (2.50 g) was purified by Prep-SFC-HPLC with the following conditions (Column: CHIRALPAK IH 3*25 cm, 5 μm; Mobile Phase A: CO₂, Mobile Phase B: MeOH (20 mM NH₃·M); Flow rate: 100 mL/min; Gradient: isocratic 30% B; Column Temperature (° C.): 35; Back Pressure (bar): 100; Wave Length: 284/220 nm; RT1 (min): 5.57; Sample Solvent: MeOH: DCM=1:2; Injection Volume: 1.2 mL), the pure fraction was concentrated then lyophilized to afford Rel-(R)-2-cyclopropyl-6-isopropyl-4-(4-(S-methylsulfonimidoyl)benzyl)-[1,2,4]triazolo[1,5-a]pyrimidin-7(4H)-one.

ESI-MS m/z=386.15 [M+H]⁺; Calculated: 385.2.

¹H NMR (400 MHZ, DMSO-d₆) δ 8.05-7.87 (m, 3H), 7.59-7.52 (m, 2H), 5.41 (s, 2H), 4.21 (s, 1H), 3.11-2.94 (m, 4H), 2.04 (tt, J=8.3, 4.9 Hz, 1H), 1.19 (d, J=6.9 Hz, 6H), 1.05-0.96 (m, 2H), 0.90 (dt, J=4.7, 3.0 Hz, 2H).

Total proton count from structure: 23.

Total proton count from spectrum: 23.

### Compound 7B: Rel-(S)-2-cyclopropyl-6-isopropyl-4-(4-(S-methylsulfonimidoyl)benzyl)-[1,2,4]triazolo[1,5-a]pyrimidin-7(4H)-one

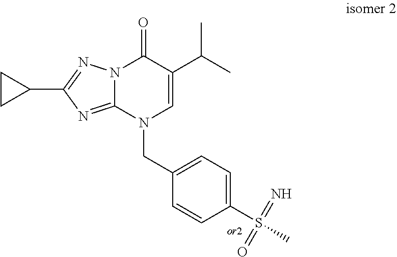 [SMILES: CC(C)c1cn(Cc2ccc([S@@](C)(=N)=O)cc2)c2nc(C3CC3)nn2c1=O]

### Step 1: (S)-2-cyclopropyl-6-isopropyl-4-(4-(S-methylsulfonimidoyl)benzyl)-[1,2,4]triazolo[1,5-a]pyrimidin-7(4H)-one

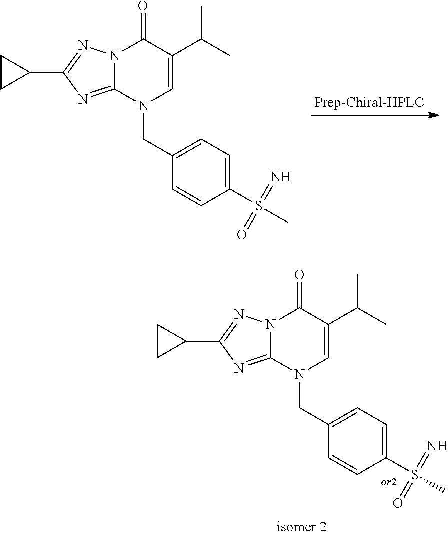 [SMILES: CC(C)c1cn(Cc2ccc(S(C)(=N)=O)cc2)c2nc(C3CC3)nn2c1=O ; CC(C)c1cn(Cc2ccc([S@@](C)(=N)=O)cc2)c2nc(C3CC3)nn2c1=O]

The racemate (150 mg) was purified by Prep-Chiral-HPLC with the following conditions (Column: CHIRALPAK-IK, 3*25 mm, 5 μm; Mobile Phase A: Hex (10 mM NH₃-MeOH), Mobile Phase B: EtOH-HPLC; Flow rate: 40 mL/min; Gradient: isocratic 50; Wave Length: 220/286 nm; RT2 (min): 21.7; Sample Solvent: MeOH:DCM=1:1-HPLC; Injection Volume: 0.5 mL; Number Of Runs: 4), the pure fraction was concentrated then lyophilized to afford(S)-2-cyclopropyl-6-isopropyl-4-(4-(S-methylsulfonimidoyl)benzyl)-[1,2,4]triazolo[1,5-a]pyrimidin-7(4H)-one.

ESI-MS m/z=386.20. [M+H]⁺; Calculated: 385.2.

¹H NMR (400 MHZ, DMSO-d₆) δ 8.11-7.88 (m, 3H), 7.60-7.53 (m, 2H), 5.41 (s, 2H), 4.54-3.82 (m, 1H), 3.12-2.90 (m, 4H), 2.10-1.95 (m, 1H), 1.19 (d, J=6.9 Hz, 6H), 1.04-0.93 (m, 2H), 0.90 (dt, J=5.0, 3.0 Hz, 2H).

Total proton count from structure: 23.

Total proton count from spectrum: 23.

### Compound 8: 4-((2-cyclopropyl-6-isopropyl-7-oxo-[1,2,4]triazolo[1,5-a]pyrimidin-4(7H)-yl)methyl)-2-(methoxymethyl)benzenesulfonamide

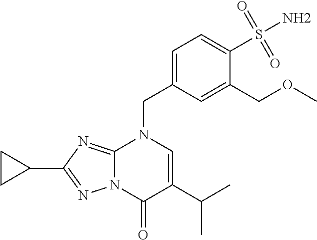 [SMILES: COCc1cc(Cn2cc(C(C)C)c(=O)n3nc(C4CC4)nc23)ccc1S(N)(=O)=O]

### Step 1: 4-(Benzylsulfanyl)-3-(hydroxymethyl)benzonitrile

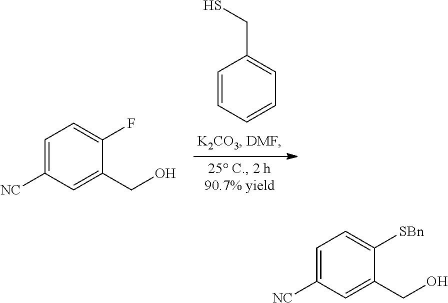

A mixed solution of K₂CO₃ (10.80 g, 78.1 mmol, 3.9 equiv), 4-fluoro-3-(hydroxymethyl)benzonitrile (3.00 g, 19.8 mmol, 1.0 equiv) and benzyl mercaptan (7.38 g, 59.4 mmol, 2.9 equiv) in 50 mL DMF was stirred for 2 h at 25° C. under N₂ atmosphere. The desired product could be detected by LCMS. The resulting mixture was diluted with EtOAc (250 mL). The resulting mixture was washed with 5×250 ml of water. The resulting mixture was concentrated under reduced pressure. The residue was purified by silica gel column chromatography, eluted with EA:PE (1:2) to afford 4-(benzylsulfanyl)-3-(hydroxymethyl)benzonitrile.

ESI-MS m/z=253.95 [M−H]⁻; Calculated: 255.1.

### Step 2: 4-(Benzylthio)-3-(methoxymethyl)benzonitrile

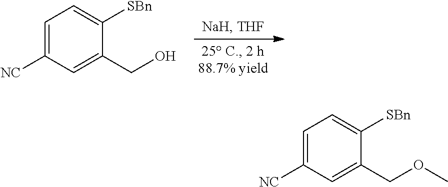

A solution of 4-(benzylsulfanyl)-3-(hydroxymethyl)benzonitrile (4.50 g, 17.6 mmol, 1.0 equiv) in THF (90 mL) was treated with NaH (2.97 g, 123.7 mmol, 7.0 equiv, 60%) for 10 min at 25° C. under nitrogen atmosphere followed by the addition of CH₃I (9.0 mL, 1.45 mol, 8.20 equiv) dropwise at 25° C. The resulting mixture was stirred for 2 h at 25° C. under N₂ atmosphere. The desired product could be detected by LCMS. The resulting mixture was filtered, and then the filter cake was washed with DCM (2×50 ml). The filtrate was concentrated under reduced pressure. The residue was purified by silica gel column with EA/PE (1:1) to afford 4-(benzylsulfanyl)-3-(methoxymethyl)benzonitrile.

ESI-MS m/z=268.00 [M−H]⁻; Calculated: 269.1

¹H NMR (400 MHZ, DMSO-d₆) δ 7.75-7.69 (m, 2H), 7.61 (d, J=8.2 Hz, 1H), 7.44-7.39 (m, 2H), 7.36-7.30 (m, 2H), 7.30-7.23 (m, 1H), 4.40-4.35 (m, 4H), 3.31 (s, 3H).

### Step 3: 4-(Benzylthio)-3-(methoxymethyl)benzaldehyde

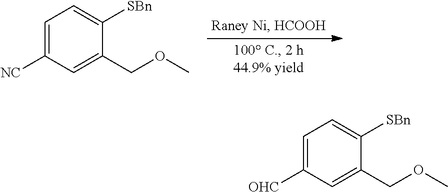

A mixture of 4-(benzylthio)-3-(methoxymethyl)benzonitrile (500 mg, 1.86 mmol, 1.0 equiv) and Raney nickel (95.4 mg, 1.11 mmol, 0.6 equiv) in HCOOH (10 mL) was stirred for 2 h at 100° C. under N₂ atmosphere. The desired product could be detected by LCMS. The resulting mixture was filtered, then the filter cake was washed with EA (50 ml×3). The filtrate was concentrated under reduced pressure. The residue was purified by reversed-phase flash chromatography with the following conditions: column: C18; mobile phase: 0.1% HCOOH water solution in MeCN, 10% to 50% gradient in 10 min; detector: UV 254 nm; to afford 4-(benzylthio)-3-(methoxymethyl)benzaldehyde.

ESI-MS m/z=273.10 [M+H]⁺; Calculated: 272.1.

### Step 4: 4-(Benzylthio)-3-(methoxymethyl)phenyl)methanol

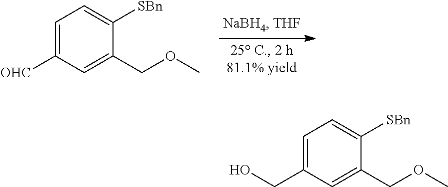

A solution of 4-(benzylthio)-3-(methoxymethyl)benzaldehyde (230 mg, 0.84 mmol, 1.0 equiv) in THF (3 mL) was stirred at 0° C. under nitrogen atmosphere followed by the addition of NaBH₄ (64 mg, 1.69 mmol, 2.0 equiv) in portions at 0° C. The resulting mixture was stirred for 2 h at 25° C. under N₂ atmosphere. The desired product could be detected by LCMS. The reaction was quenched by the addition of Water (5 ml) at 0° C. The resulting mixture was extracted with EtOAc (2×10 mL). The combined organic layers were washed with brine (1×10 mL), and dried over anhydrous Na₂SO₄. After filtration, the filtrate was concentrated under reduced pressure. The residue was purified by silica gel column chromatography, eluted with EA/PE (1:3) to afford 4-(Benzylthio)-3-(methoxymethyl)phenyl)methanol.

ESI-MS m/z=257.10 [M-OH]⁺; Calculated: 274.1

### Step 5: Benzyl(4-(bromomethyl)-2-(methoxymethyl)phenyl) sulfane

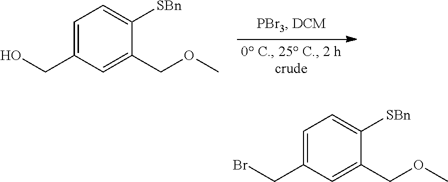

A solution of 4-(benzylthio)-3-(methoxymethyl)phenyl)methanol (188 mg, 0.69 mmol, 1.0 equiv) in DCM (3 mL) was stirred at 0° C. under nitrogen atmosphere followed by the addition of PBr₃ (556 mg, 2.06 mmol, 3.0 equiv) at 0° C. The resulting mixture was stirred for 2 h at 25° C. under N₂ atmosphere. The desired product could be detected by LCMS. The resulting mixture was extracted with DCM. The combined organic layers were washed with brine water, and dried over anhydrous Na₂SO₄. After filtration, the filtrate was concentrated under reduced pressure. The crude product resulting mixture was used in the next step directly without further purification.

ESI-MS m/z=257.10 [M-Br]⁺; Calculated: 336.0. & 338.0.

### Step 6: 4-(4-(Benzylthio)-3-(methoxymethyl)benzyl)-2-cyclopropyl-6-isopropyl-[1,2,4]triazolo[1,5-a]pyrimidin-7(4H)-one

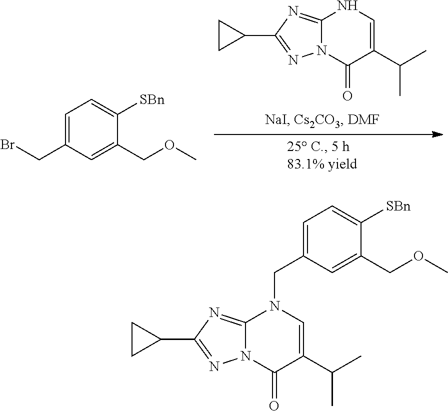

A mixed solution of Cs₂CO₃ (413 mg, 1.27 mmol, 2.0 equiv), NaI (38 mg, 0.25 mmol, 0.40 equiv), benzyl(4-(bromomethyl)-2-(methoxymethyl)phenyl) sulfane (214 mg, 0.64 mmol, 1.0 equiv) and 2-cyclopropyl-6-isopropyl-4H-[1,2,4]triazolo[1,5-a]pyrimidin-7-one (180 mg, 0.83 mmol, 1.3 equiv) in DMF (5 mL) was stirred for 5 h at 25° C. under N₂ atmosphere. The desired product could be detected by LCMS. The resulting mixture was diluted with EtOAc (25 mL). The resulting mixture was washed with 5×25 ml of water. The combined organic layers were dried over anhydrous Na₂SO₄. After filtration, the filtrate was concentrated under reduced pressure. The residue was purified by silica gel column chromatography, eluted with EA/PE (1:2) to afford 4-(4-(benzylthio)-3-(methoxymethyl)benzyl)-2-cyclopropyl-6-isopropyl-[1,2,4]triazolo[1,5-a]pyrimidin-7(4H)-one.

ESI-MS m/z=475.30 [M+H]⁺; Calculated: 474.2.

### Step 7: 4-((2-Cyclopropyl-6-isopropyl-7-oxo-[1,2,4]triazolo[1,5-a]pyrimidin-4(7H)-yl)methyl)-2-(methoxymethyl)benzenesulfonyl chloride

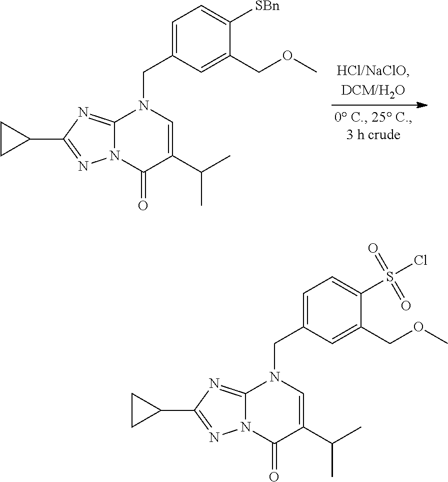

A solution of 4-(4-(benzylthio)-3-(methoxymethyl)benzyl)-2-cyclopropyl-6-isopropyl-[1,2,4]triazolo[1,5-a]pyrimidin-7(4H)-one (250 mg, 0.53 mmol, 1.0 equiv) in mixed solvent of DCM (3 mL) and H₂O (1 mL) was treated with HCl (0.5 mL, 5.27 mmol, 10.0 equiv, 36 wt %) for 3 min at 0° C. under N₂ atmosphere followed by the addition of NaClO (1.5 mL, 1.58 mmol, 3.0 equiv, 8 wt %) dropwise at 0° C. The resulting mixture was stirred for 3 h at 25° C. under inert atmosphere. The desired product could be detected by LCMS. The resulting mixture was extracted with DCM. The combined organic layers were washed with saturated brine water, and dried over anhydrous Na₂SO₄. After filtration, the filtrate was concentrated under reduced pressure. The obtained crude product was used in the next step directly without further purification.

ESI-MS m/z=451.10 [M+H]⁺; Calculated: 450.1.

### Step 8: 4-((2-Cyclopropyl-6-isopropyl-7-oxo-[1,2,4]triazolo[1,5-a]pyrimidin-4(7H)-yl)methyl)-2-(methoxymethyl)benzenesulfonamide

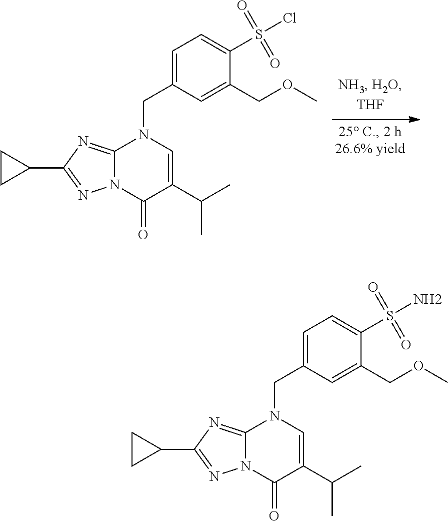 [SMILES: COCc1cc(Cn2cc(C(C)C)c(=O)n3nc(C4CC4)nc23)ccc1S(=O)(=O)Cl ; COCc1cc(Cn2cc(C(C)C)c(=O)n3nc(C4CC4)nc23)ccc1S(N)(=O)=O]

To the above mixture was added ammonia (2 mL, 30% wt) and the reaction was stirred for 2 h at 25° C. under inert atmosphere. The desired product could be detected by LCMS. The reaction was diluted by water (5 mL). The resulting mixture was extracted with DCM (2×40 mL). The combined organic layers were washed with saturated brine water, and dried over anhydrous Na₂SO₄. After filtration, the filtrate was concentrated under reduced pressure. The crude product was purified by Prep-HPLC with the following conditions: (Column: XBridge Shield RP18 OBD Column 30*150 mm, 5 m; Mobile Phase A: Water (0.1% FA), Mobile Phase B: ACN; Flow rate: 60 mL/min mL/min; Gradient: 25% B to 41% B in 8 min; Wave Length: 254 nm/220 nm nm; RT1 (min): 9.45) the pure fraction was concentrated then lyophilized to afford 4-(2-cyclopropyl-6-isopropyl-7-oxo-[1,2,4]triazolo[1,5-a]pyrimidin-4(7H)-yl)methyl)-2-(methoxymethyl)benzene sulfonamide.

ESI-MS m/z=432.05 [M+H]⁺; Calculated: 431.2.

¹H NMR (400 MHZ, DMSO-d₆) δ 8.07 (s, 1H), 7.82 (d, J=8.1 Hz, 1H), 7.71 (s, 1H), 7.44 (s, 2H), 7.42 (d, J=1.9 Hz, 1H), 5.39 (s, 2H), 4.79 (s, 2H), 3.42-3.35 (m, 3H), 3.05-2.93 (m, 1H), 2.04 (m, 1H), 1.18 (dd, J=6.9 Hz, 6H), 1.04-0.98 (m, 2H), 0.94-0.89 (m, 2H).

Total proton counts from structure: 25.

Total proton counts from spectrum: 25.

### Compound 9 Rac: 6-isopropyl-2-methyl-4-(4-(S-methylsulfonimidoyl)benzyl)pyrazolo[1,5-a]pyrimidin-7(4H)-one

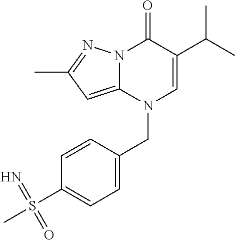 [SMILES: Cc1cc2n(Cc3ccc(S(C)(=N)=O)cc3)cc(C(C)C)c(=O)n2n1]

### Step 1: 6-Isopropyl-2-methyl-4-(4-(S-methylsulfonimidoyl)benzyl)pyrazolo[1,5-a]pyrimidin-7(4H)-one

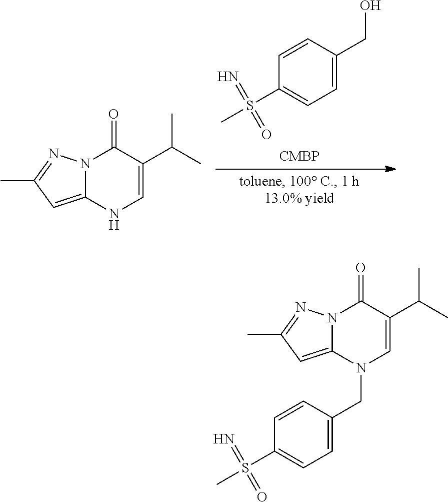

To a stirred mixture of 6-isopropyl-2-methyl-4H-pyrazolo[1,5-a]pyrimidin-7-one (300 mg, 1.57 mmol, 1.0 equiv) and [4-(hydroxymethyl)phenyl](imino)methyl-lambda6-sulfanone (291 mg, 1.57 mmol, 1.0 equiv) in Toluene (6 mL) was added CMBP (757 mg, 3.14 mmol, 2.0 equiv) dropwise at 25° C. The resulting mixture was stirred for 1 h at 100° C. under nitrogen atmosphere. The desired product could be detected by LCMS. The mixture was allowed to cool down to 25° C. The resulting mixture was concentrated under reduced pressure. The residue was purified by reversed-phase flash chromatography with the following conditions: column: C18; mobile phase: MeCN in Water (0.1% NH₃·H₂O), 15% to 55% gradient in 20 min; detector: UV 254 nm. This resulted in crude product (200 mg). The crude product was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min; Gradient: 5% B to 32% B in 7 min; Wave Length: 254 nm/220 nm; RT1 (min): 6.88), the pure factions were concentrated under vacuum then lyophilized to afford 6-isopropyl-2-methyl-4-(4-(S-methylsulfonimidoyl)benzyl)pyrazolo[1,5-a]pyrimidin-7(4H)-one.

ESI-MS m/z=359.10 [M+H]⁺; Calculated: 358.1.

¹H NMR (400 MHZ, DMSO-d₆) δ 8.00 (s, 1H), 7.93 (d, J=8.4 Hz, 2H), 7.54 (d, J=8.4 Hz, 2H), 6.04 (s, 1H), 5.37 (s, 2H), 4.23 (s, 1H), 3.06 (s, 4H), 2.24 (s, 3H), 1.21 (d, J=6.9 Hz, 6H).

Total proton count from structure: 22.

Total proton count from spectrum: 22.

### Compound 9A: Rel-(S)-6-isopropyl-2-methyl-4-(4-(S-methylsulfonimidoyl)benzyl)pyrazolo[1,5-a]pyrimidin-7(4H)-one

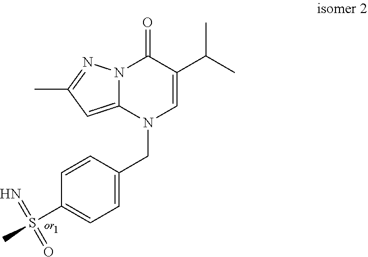 [SMILES: Cc1cc2n(Cc3ccc([S@@](C)(=N)=O)cc3)cc(C(C)C)c(=O)n2n1]

### Step 1: Rel-(S)-6-isopropyl-2-methyl-4-(4-(S-methylsulfonimidoyl)benzyl)pyrazolo[1,5-a]pyrimidin-7(4H)-one

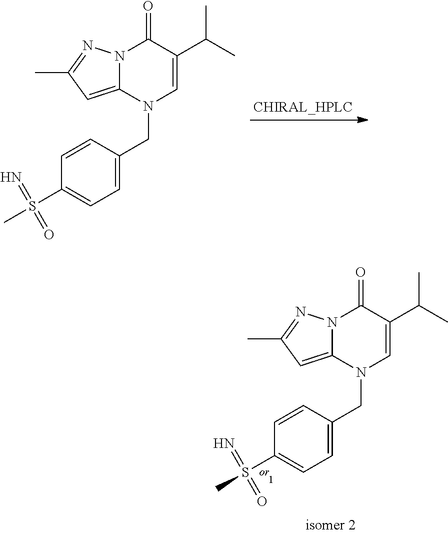 [SMILES: Cc1cc2n(Cc3ccc(S(C)(=N)=O)cc3)cc(C(C)C)c(=O)n2n1 ; Cc1cc2n(Cc3ccc([S@@](C)(=N)=O)cc3)cc(C(C)C)c(=O)n2n1]

The racemate (67 mg) was separated by PREP-CHIRAL-HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50; Wave Length: 202/220 nm; RT1 (min): 11.7; Sample Solvent: EtOH:DCM=2:1; Injection Volume: 0.4 mL; Number Of Runs: 9), the pure factions were concentrated under vacuum then lyophilized to afford rel-(S)-6-isopropyl-2-methyl-4-(4-(S-methylsulfonimidoyl)benzyl)pyrazolo[1,5-a]pyrimidin-7(4H)-one.

ESI-MS m/z=359.00 [M+H]⁺; Calculated: 358.1.

¹H NMR (400 MHZ, DMSO-d₆) δ 7.98 (s, 1H), 7.94-7.85 (m, 2H), 7.52 (d, J=8.3 Hz, 2H), 6.03 (s, 1H), 5.35 (s, 2H), 4.28 (s, 1H), 3.09-2.96 (m, 4H), 2.28-2.18 (m, 3H), 1.21 (d, J=6.8 Hz, 6H).

Total proton count from structure: 22.

Total proton count from spectrum: 22.

### Compound 9B: Rel-(R)-6-isopropyl-2-methyl-4-(4-(S-methylsulfonimidoyl)benzyl)pyrazolo[1,5-a]pyrimidin-7(4H)-one

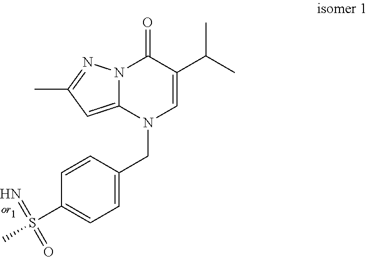 [SMILES: Cc1cc2n(Cc3ccc([S@](C)(=N)=O)cc3)cc(C(C)C)c(=O)n2n1]

### Step 1: Rel-(R)-6-isopropyl-2-methyl-4-(4-(S-methylsulfonimidoyl)benzyl)pyrazolo[1,5-a]pyrimidin-7(4H)-one

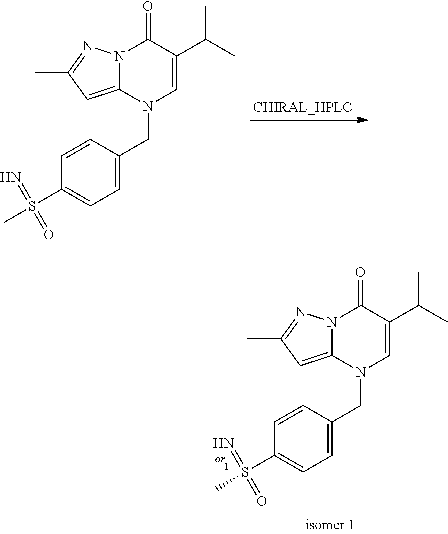 [SMILES: Cc1cc2n(Cc3ccc(S(C)(=N)=O)cc3)cc(C(C)C)c(=O)n2n1 ; Cc1cc2n(Cc3ccc([S@](C)(=N)=O)cc3)cc(C(C)C)c(=O)n2n1]

The racemate (67 mg) was separated by PREP-CHIRAL-HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50; Wave Length: 202/220 nm; RT2 (min): 13.7; Sample Solvent: EtOH:DCM=2:1; Injection Volume: 0.4 mL; Number Of Runs: 9), the pure factions were concentrated under vacuum then lyophilized to afford rel-(R)-6-isopropyl-2-methyl-4-(4-(S-methylsulfonimidoyl)benzyl)pyrazolo[1,5-a]pyrimidin-7(4H)-one.

ESI-MS m/z=359.00 [M+H]⁺; Calculated: 358.1.

¹H NMR (400 MHZ, DMSO-d₆) δ 8.01-7.94 (m, 1H), 7.94-7.88 (m, 2H), 7.55-7.49 (m, 2H), 6.06-5.99 (m, 1H), 5.34 (s, 2H), 4.23 (s, 1H), 3.09-2.93 (m, 4H), 2.27-2.18 (m, 3H), 1.27-1.13 (m, 6H).

Total proton count from structure: 22.

Total proton count from spectrum: 22.

### Compound 10: 6-((2-cyclopropyl-6-isopropyl-7-oxo-[1,2,4]triazolo[1,5-a]pyrimidin-4(7H)-yl)methyl)-2-methylpyridine-3-sulfonamide

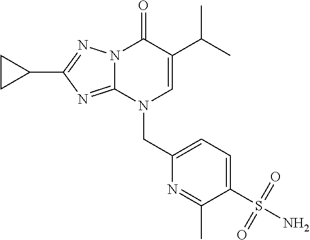 [SMILES: Cc1nc(Cn2cc(C(C)C)c(=O)n3nc(C4CC4)nc23)ccc1S(N)(=O)=O]

### Step 1: 5-(Benzylthio)-6-methylpicolinonitrile

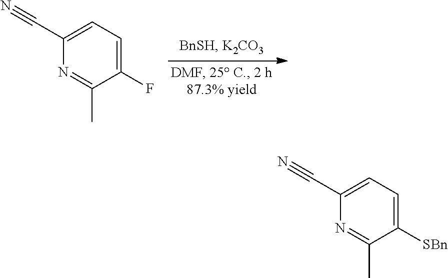

To a stirred solution of 5-fluoro-6-methylpyridine-2-carbonitrile (3.50 g, 25.7 mmol, 1.0 equiv) and phenylmethanethiol (3.83 g, 30.9 mmol, 1.2 equiv) in DMF (50 mL) was added K₂CO₃ (7.11 g, 51.4 mmol, 2.0 equiv) in portions at 25° C. under nitrogen atmosphere. The resulting mixture was stirred for 1 h at 25° C. under nitrogen atmosphere. The desired product could be detected by LCMS. The residue was purified by trituration with water (150 mL). The precipitated solids were collected by filtration and washed with water (3×10 mL). The resulting mixture was concentrated under reduced pressure. The resulting mixture was concentrated under vacuum to afford 5-(Benzylthio)-6-methylpicolinonitrile.

ESI-MS m/z=241.05. [M+H]⁺; Calculated MW: 240.1.

¹H NMR (300 MHZ, DMSO-d₆) δ 7.93-7.79 (m, 2H), 7.51-7.40 (m, 2H), 7.40-7.24 (m, 3H), 4.42 (s, 2H), 2.44 (s, 3H).

### Step 2: 6-Cyano-2-methylpyridine-3-sulfonyl chloride

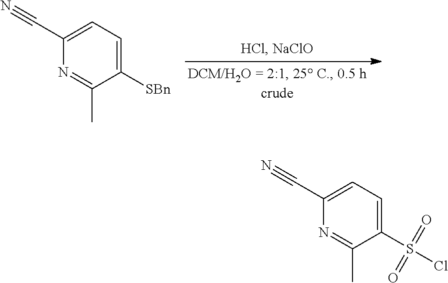

To a stirred solution of 5-(benzylsulfanyl)-6-methylpyridine-2-carbonitrile (5.00 g, 20.8 mmol, 1.0 equiv) in DCM (150 mL) and H₂O (75 mL) was added conc.HCl (30 mL) dropwise at 0° C. under nitrogen atmosphere. To the above mixture was added NaClO (75 mL, 8% wt in H₂O) dropwise at 0° C. The resulting mixture was stirred for an additional 0.5 h at 0° C. The desired product could be detected by LCMS. The reaction was quenched with water at 0° C. The resulting mixture was diluted with water (100 mL). The resulting mixture was extracted with CH₂Cl₂ (3×150 mL). The combined organic layers were washed with brine (200 mL), and dried over anhydrous Na₂SO₄. After filtration, the filtrate was concentrated under reduced pressure to afford 6-cyano-2-methylpyridine-3-sulfonyl chloride (crude). The crude product mixture was used in the next step directly without further purification.

### Step 3: 6-Cyano-2-methylpyridine-3-sulfonamide

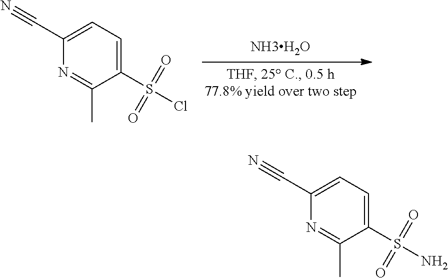

To a stirred solution of 6-cyano-2-methylpyridine-3-sulfonyl chloride (7.00 g, 23.1 mmol, 1.0 equiv) in THF (80 mL) was added NH₃·H₂O (5 mL, 30% wt. in H₂O) dropwise at 25° C. under nitrogen atmosphere. The resulting mixture was stirred for 0.5 h at 25° C. under nitrogen atmosphere. The desired product could be detected by LCMS. The resulting mixture was concentrated under reduced pressure. The resulting mixture was concentrated under vacuum. The residue was purified by silica gel column chromatography, eluted with CH₂Cl₂/MeOH (0% to 5% gradient in 20 min) to afford 6-cyano-2-methylpyridine-3-sulfonamide.

ESI-MS m/z=198.05. [M+H]⁺; Calculated MW: 197.0

¹H NMR (300 MHZ, DMSO-d₆) δ 8.37 (d, J=8.1 Hz, 1H), 8.14-8.07 (m, 1H), 7.94 (s, 2H), 2.82 (s, 3H).

### Step 4: 6-Methyl-5-sulfamoylpicolinic acid

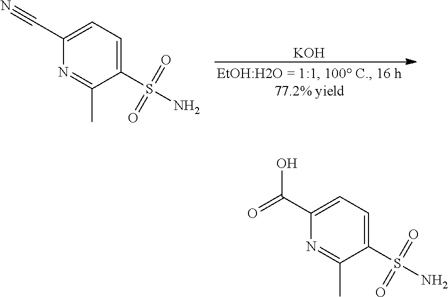

To a stirred solution of 6-cyano-2-methylpyridine-3-sulfonamide (1.50 g, 7.61 mmol, 1.0 equiv) and KOH (1.71 g, 30.4 mmol, 4.0 equiv) in EtOH (15 mL) was added H₂O (15 mL) at 25° C. under nitrogen atmosphere. The resulting mixture was stirred for 16 h at 100° C. under nitrogen atmosphere. The desired product could be detected by LCMS. The mixture was allowed to cool down to 25° C. The resulting mixture was diluted with water (30 mL). The mixture/residue was acidified to pH 4 with HCl (aq.). The resulting mixture was extracted with EtOAc (3×50 mL). The combined organic layers were washed with brine (100 mL), and dried over anhydrous Na₂SO₄. After filtration, the filtrate was concentrated under reduced pressure to afford 6-methyl-5-sulfamoylpyridine-2-carboxylic acid.

ESI-MS m/z=216.95. [M+H]⁺; Calculated MW: 216.0.

¹H NMR (400 MHZ, DMSO-d₆) δ 13.50 (br, 1H), 8.32 (d, J=8.1 Hz, 1H), 8.04 (d, J=8.1 Hz, 1H), 7.80 (s, 2H), 2.82 (s, 3H).

### Step 5: 6-(Hydroxymethyl)-2-methylpyridine-3-sulfonamide

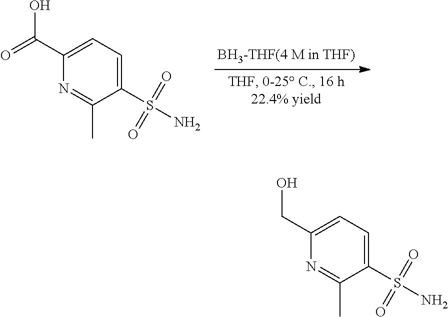

To a stirred solution of 6-methyl-5-sulfamoylpyridine-2-carboxylic acid (300 mg, 1.39 mmol, 1.0 equiv) in THF (8 mL) was added BH₃-THF (8.3 mL, 8.33 mmol, 6.0 equiv, 1 M in THF) dropwise at 0° C. under nitrogen atmosphere. The resulting mixture was stirred for 16 h at 25° C. under nitrogen atmosphere. The desired product could be detected by LCMS. The reaction was quenched with MeOH at 0° C. The resulting mixture was concentrated under vacuum. The residue was purified by silica gel column chromatography, eluted with CH₂Cl₂/MeOH (0%-10%, 20 min) to afford 6-(hydroxymethyl)-2-methylpyridine-3-sulfonamide.

ESI-MS m/z=203.00. [M+H]⁺; Calculated MW: 202.0.

### Step 6: 6-((2-Cyclopropyl-6-isopropyl-7-oxo-[1,2,4]triazolo[1,5-a]pyrimidin-4(7H)-yl)methyl)-2-methylpyridine-3-sulfonamide

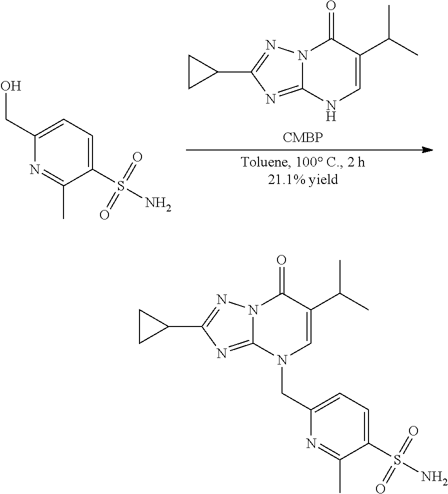 [SMILES: Cc1nc(CO)ccc1S(N)(=O)=O ; CC(C)c1c[nH]c2nc(C3CC3)nn2c1=O ; Cc1nc(Cn2cc(C(C)C)c(=O)n3nc(C4CC4)nc23)ccc1S(N)(=O)=O]

To a stirred solution of 6-(hydroxymethyl)-2-methylpyridine-3-sulfonamide (60 mg, 0.30 mmol, 1.0 equiv) and 2-cyclopropyl-6-isopropyl-4H-[1,2,4]triazolo[1,5-a]pyrimidin-7-one (65 mg, 0.30 mmol, 1.0 equiv) in Toluene (5 mL) was added CMBP (716 mg, 2.97 mmol, 10.0 equiv) at 25° C. under nitrogen atmosphere. The resulting mixture was stirred for 2 h at 100° C. under nitrogen atmosphere. The desired product could be detected by LCMS. The mixture was allowed to cool down to 25° C. The resulting mixture was concentrated under reduced pressure. The residue was purified by Prep-TLC (CH₂Cl₂/MeOH=10:1) to afford the crude product (80 mg). The crude product was purified by Prep-HPLC with the following conditions (Column: XBridge Prep OBD C18 Column, 30*150 mm, 10 μm; Mobile Phase A: water (10 mmol/L NH₄HCO₃+0.05% NH₃H₂O), Mobile Phase B: ACN; Flow rate: 60 mL/min; Gradient: 18% B to 33% B in 8 min; Wave Length: 254 nm/220 nm nm; RT1 (min): 9.60), the pure fraction was concentrated then lyophilized to afford 6-((2-cyclopropyl-6-isopropyl-7-oxo-[1,2,4]triazolo[1,5-a]pyrimidin-4(7H)-yl)methyl)-2-methylpyridine-3-sulfonamide.

ESI-MS m/z=403.15. [M+H]⁺; Calculated MW: 402.1.

¹H NMR (300 MHz, DMSO-d₆) δ 8.13 (d, J=8.1 Hz, 1H), 7.99 (s, 1H), 7.61 (br, 2H), 7.31 (d, J=8.2 Hz, 1H), 5.47 (s, 2H), 3.09-2.96 (m, 1H), 2.70 (s, 3H), 2.10-1.93 (m, 1H), 1.20 (d, J=6.9 Hz, 6H), 1.01-0.90 (m, 2H), 0.90-0.77 (m, 2H).

Total proton count from structure: 22.

Total proton count from spectrum: 22.

### Compound 11: 4-((2-cyclopropyl-6-isopropyl-7-oxo-[1,2,4]triazolo[1,5-a]pyrimidin-4(7H)-yl)methyl)benzenesulfonamide

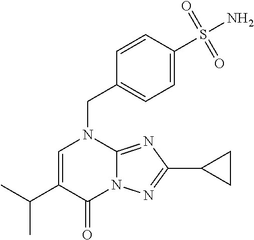 [SMILES: CC(C)c1cn(Cc2ccc(S(N)(=O)=O)cc2)c2nc(C3CC3)nn2c1=O]

### Step 1: 4-((2-Cyclopropyl-6-isopropyl-7-oxo-[1,2,4]triazolo[1,5-a]pyrimidin-4(7H)-yl)methyl)benzenesulfonamide

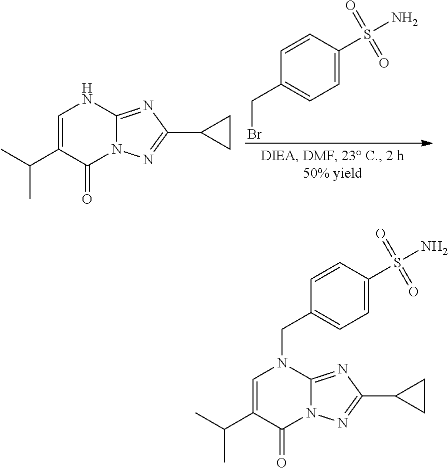

To a stirred solution of 2-cyclopropyl-6-isopropyl-4H-[1,2,4]triazolo[1,5-a]pyrimidin-7-one (3.00 g, 13.7 mmol, 1.0 equiv) and 4-(bromomethyl)benzenesulfonamide (3.44 g, 13.7 mmol, 1.0 equiv) in DMF (30 mL) was added DIEA (5.33 g, 41.2 mmol, 3.0 equiv) at 23° C. under nitrogen atmosphere. The resulting mixture was stirred for 2 h at 23° C. under nitrogen atmosphere. The reaction was monitored by LCMS.

The resulting mixture was concentrated under reduced pressure. The residue was dissolved in DMF (15 mL). The resulting mixture was precipitated by the addition of water (150 ml). The precipitated solids were collected by filtration and washed with water (3×15 mL). The crude product was re-crystallized from DMF/water (1:30 300 mL) to afford 4-((2-Cyclopropyl-6-isopropyl-7-oxo-[1,2,4]triazolo[1,5-a]pyrimidin-4(7H)-yl)methyl)benzenesulfonamide.

ESI-MS m/z=388.15 [M+H]⁺; Calculated: 387.1.

¹H NMR (400 MHZ, DMSO-d₆) δ 8.02 (s, 1H), 7.84-7.76 (m, 2H), 7.56-7.50 (m, 2H), 7.34 (s, 2H), 5.39 (s, 2H), 3.02-2.95 (m, 1H), 2.07-2.01 (m, 1H), 1.19 (d, J=6.9 Hz, 6H), 1.02-0.98 (m, 2H), 0.89-0.86 (m, 2H).

Total proton count from structure: 21

Total proton count from spectrum: 21

### Compound 12: 2-cyclopropyl-4-(2-fluoro-4-(S-methylsulfonimidoyl)benzyl)-6-isopropyl-[1,2,4]triazolo[1,5-a]pyrimidin-7(4H)-one

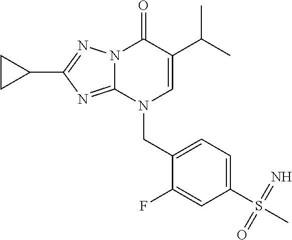 [SMILES: CC(C)c1cn(Cc2ccc(S(C)(=N)=O)cc2F)c2nc(C3CC3)nn2c1=O]

### Step 1: 2-Fluoro-4-(methylthio)benzonitrile

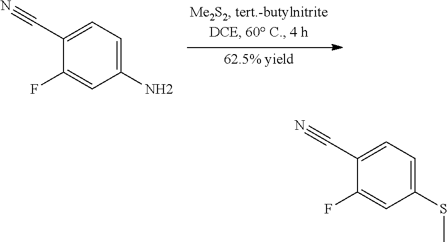

A solution of 4-amino-2-fluorobenzonitrile (5.00 g, 36.7 mmol, 1.0 equiv), tert.-butylnitrite (5.11 g, 49.6 mmol, 1.4 equiv) and dimethyl disulfide (4.67 g, 49.6 mmol, 1.4 equiv) in 1,2-dichloroethane (208 mL) was stirred for 4 h at 60° C. under nitrogen atmosphere. The reaction was monitored by TLC (CH₂Cl₂/PE=1:5, Rf=0.2). The resulting mixture was concentrated under reduced pressure. The residue was purified by silica gel column chromatography, eluted with CH₂Cl₂/PE (1:5) to afford 2-fluoro-4-(methylsulfanyl)benzonitrile.

¹H NMR (400 MHZ, DMSO-d₆) δ 7.78 (dd, J=8.3, 7.1 Hz, 1H), 7.40 (dd, J=10.7, 1.8 Hz, 1H), 7.26 (dd, J=8.3, 1.8 Hz, 1H), 2.57 (s, 3H)

### Step 2: 2-Fluoro-4-(methylthio)benzoic acid

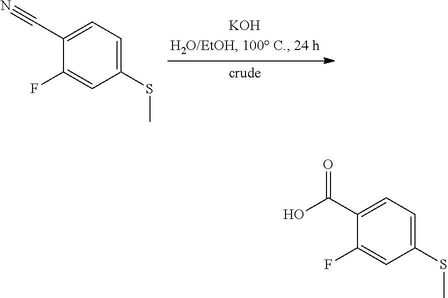

A solution of 2-fluoro-4-(methylsulfanyl)benzonitrile (4.36 g, 26.1 mmol, 1.0 equiv) and KOH (14.6 g, 261 mmol, 10 equiv) in H₂O (100 mL) and EtOH (100 mL) was stirred for 24 h at 90° C. under nitrogen atmosphere. The desired product could be detected by LCMS. The mixture was allowed to cool down to room temperature. The mixture was acidified to pH 3 with HCl (aq.) and a large amount of white solid precipitate. The precipitated solid was collected by filtration and washed with deionized water to afford 2-fluoro-4-(methylsulfanyl)benzoic acid (crude).

ESI-MS m/z=184.95 [M−H]⁻; Calculated MW: 186.0.

### Step 3: (2-Fluoro-4-(methylthio)phenyl)methanol

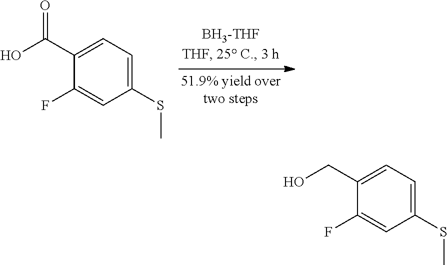

To a stirred solution of 2-fluoro-4-(methylsulfanyl)benzoic acid (2.70 g, 14.5 mmol, 1.0 equiv) in THF (20 mL) was added BH₃-THF (43.5 mL, 43.5 mmol, 3.0 equiv, 1 M in THF) dropwise at 0° C. under nitrogen atmosphere. The resulting mixture was stirred for an additional 3 h at 25° C. The reaction was monitored by LCMS. The resulting mixture was concentrated under reduced pressure. The residue was purified by silica gel column chromatography, eluted with PE/EA (3:1-1:1) to afford [2-fluoro-4-(methylsulfanyl)phenyl]methanol.

ESI-MS m/z=155.0 [M-17]⁺; Calculated MW: 172.0.

¹H NMR (400 MHZ, DMSO-d₆) δ 7.41-7.34 (m, 1H), 7.10-7.07 (m, 1H), 7.06 (dd, J=6.5, 1.7 Hz, 1H), 5.22 (t, J=5.7 Hz, 1H), 4.49 (dt, J=5.7, 1.0 Hz, 2H), 2.48 (s, 3H).

### Step 4: (3-Fluoro-4-(hydroxymethyl)phenyl)(imino)(methyl)-λ⁶-sulfanone

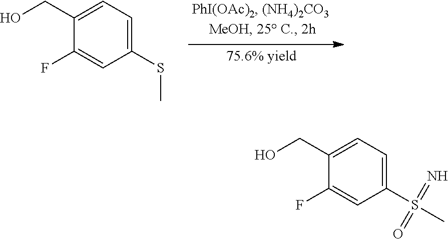

To a stirred solution of [2-fluoro-4-(methylsulfanyl)phenyl]methanol (1.20 g, 6.97 mmol, 1.0 equiv) and (diacetoxyiodo)benzene (5.84 g, 18.1 mmol, 2.6 equiv) in MeOH (45 mL) was added ammonium carbonate (880 mg, 9.15 mmol, 1.5 equiv) in portions at 25° C. under air atmosphere. The resulting mixture was stirred for an additional 2 h at 25° C. The desired product could be detected by LCMS. The resulting mixture was concentrated under reduced pressure. The residue was purified by silica gel column chromatography, eluted with CH₂Cl₂/MeOH (0-10:1) to afford [3-fluoro-4-(hydroxymethyl)phenyl](imino)methyl-λ⁶-sulfanone.

ESI-MS m/z=203.9 [M+H]⁺; Calculated MW: 203.0.

### Step 5: (4-(Bromomethyl)-3-fluorophenyl)(imino)(methyl)-λ⁶-sulfanone

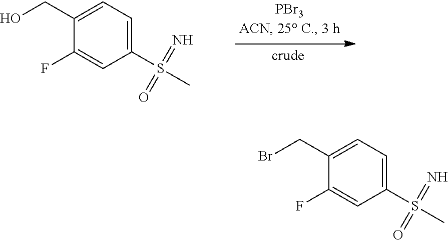

To a stirred solution of [3-fluoro-4-(hydroxymethyl)phenyl](imino)methyl-λ⁶-sulfanone (1.07 g, 5.26 mmol, 1.0 equiv) in DCM (23 mL) was added PBr₃ (2.14 g, 7.89 mmol, 1.5 equiv) dropwise at 0° C. under air atmosphere. The resulting mixture was stirred for an additional 3 h at 25° C. and white solid precipitate. The precipitated solids were collected by filtration and washed with CH₂Cl₂ to afford [4-(bromomethyl)-3-fluorophenyl](imino)methyl-λ⁶-sulfanone (crude). The crude product was used in the next step directly without further purification.

ESI-MS m/z=265.8&267.8 [M+H]⁺; Calculated MW: 265.0&267.0.

### Step 6: 2-Cyclopropyl-4-(2-fluoro-4-(S-methylsulfonimidoyl)benzyl)-6-isopropyl-[1,2,4]triazolo[1,5-a]pyrimidin-7(4H)-one

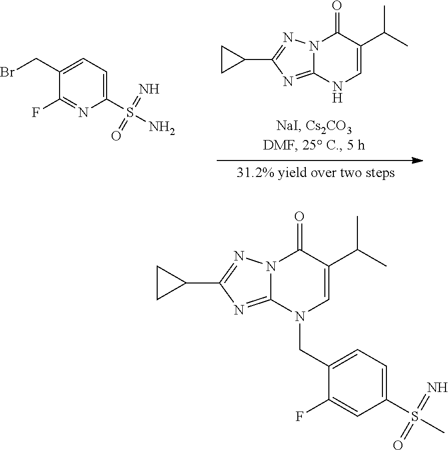 [SMILES: N=S(N)(=O)c1ccc(CBr)c(F)n1 ; CC(C)c1c[nH]c2nc(C3CC3)nn2c1=O ; CC(C)c1cn(Cc2ccc(S(C)(=N)=O)cc2F)c2nc(C3CC3)nn2c1=O]

A solution of [4-(bromomethyl)-3-fluorophenyl](imino)methyl-λ⁶-sulfanone (200 mg, 0.75 mmol, 1.0 equiv). 2-cyclopropyl-6-isopropyl-4H-[1,2,4]triazolo[1,5-a]pyrimidin-7-one (179 mg, 0.83 mmol, 1.1 equiv), NaI (113 mg, 0.75 mmol, 1.0 equiv) and Cs₂CO₃ (734 mg, 2.26 mmol, 3.0 equiv) in DMF (12.00 mL) was stirred for 5 h at room temperature under air atmosphere. The desired product could be detected by LCMS. The resulting mixture was diluted with brine (120 mL). The resulting mixture was extracted with EtOAc (2×30 mL). The combined organic layers were washed with brine (2×20 mL), and dried over anhydrous Na₂SO₄. After filtration, the filtrate was concentrated under reduced pressure. The residue was purified by reversed-phase flash chromatography with the following conditions: (Column: XBridge Prep Phenyl OBD Column 19*250 mm, 5 m; Mobile Phase A: 10 mmol/L NH₄HCO₃+0.05% NH₃·H₂O), Mobile Phase B: Acetonitrile; Flow rate: 60 mL/min mL/min; Gradient: 5% B to 5% B in 1 min, 5% B to 20% B in 2 min, 20% to 38% B in 10 min; Wave Length: 254 nm/220 nm nm; RT1 (min): 8.33), the pure fraction was concentrated then lyophilized to afford 2-cyclopropyl-4-(2-fluoro-4-(S-methylsulfonimidoyl)benzyl)-6-isopropyl-[1,2,4]triazolo[1,5-a]pyrimidin-7(4H)-one.

ESI-MS m/z=404.20 [M+H]⁺; Calculated MW: 403.1.

¹H NMR (400 MHZ, DMSO-d₆) δ 7.99 (s, 1H), 7.84-7.67 (m, 2H), 7.51 (t, J=7.6 Hz, 1H), 5.46 (s, 2H), 4.40 (s, 1H), 3.10 (s, 3H), 3.06-2.89 (m, 1H), 2.09-1.93 (m, 1H), 1.19 (d, J=6.9 Hz, 6H), 1.03-0.92 (m, 2H), 0.91-0.80 (m, 2H).

¹⁹F NMR (377 MHz, DMSO) δ −114.96.

Total proton counts from structure: 22

Total proton counts from spectrum: 22

### Compound 13: 5-((2-cyclopropyl-6-isopropyl-7-oxo-[1,2,4]triazolo[1,5-a]pyrimidin-4(7H)-yl)methyl)pyridine-2-sulfonamide

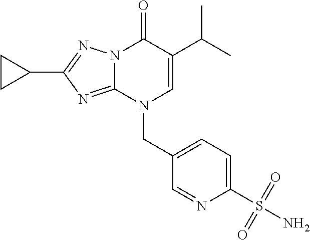 [SMILES: CC(C)c1cn(Cc2ccc(S(N)(=O)=O)nc2)c2nc(C3CC3)nn2c1=O]

### Step 1: 5-(Bromomethyl)pyridine-2-sulfonamide

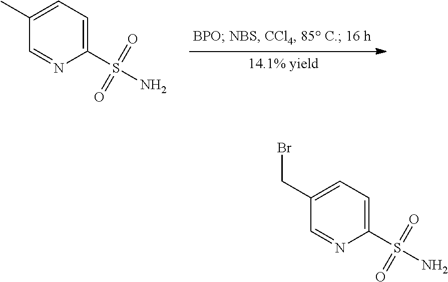

A solution of 5-methylpyridine-2-sulfonamide (5.00 g, 29.0 mmol, 1.0 equiv), NBS (7.75 g, 43.5 mmol, 1.5 equiv) and BPO (1.49 g, 5.80 mmol, 0.20 equiv) in CCl₄ (50 mL) was stirred for 16 h at 85° C. under nitrogen atmosphere. The reaction was monitored by LCMS. The mixture was allowed to cool down to 25° C. The resulting mixture was diluted with EtOAc (100 mL). The residue was washed with water (5×100 mL). The residue was purified by silica gel column chromatography, eluted with PE/EA (1:1) to afford 5-(bromomethyl)pyridine-2-sulfonamide.

ESI-MS m/z=251.1 [M+H]⁺; Calculated: 249.9.

### Step 2: 5-((2-Cyclopropyl-6-isopropyl-7-oxo-[1,2,4]triazolo[1,5-a]pyrimidin-4(7H)-yl)methyl)pyridine-2-sulfonamide

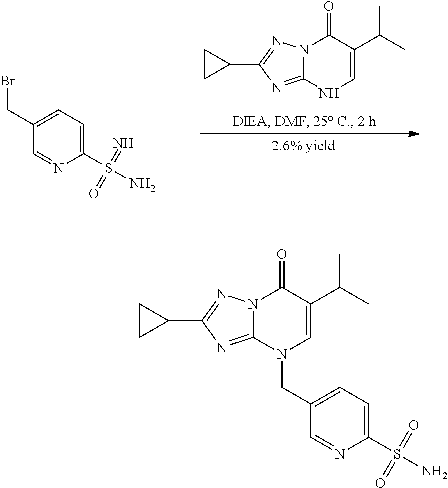 [SMILES: N=S(N)(=O)c1ccc(CBr)cn1 ; CC(C)c1c[nH]c2nc(C3CC3)nn2c1=O ; CC(C)c1cn(Cc2ccc(S(N)(=O)=O)nc2)c2nc(C3CC3)nn2c1=O]

A solution of 5-(bromomethyl)pyridine-2-sulfonamide (600 mg, 2.38 mmol, 1.0 equiv), 2-cyclopropyl-6-isopropyl-4H-[1,2,4]triazolo[1,5-a]pyrimidin-7-one (469 mg, 2.15 mmol, 0.90 equiv) and DIEA (926 mg, 7.16 mmol, 3.0 equiv) in DMF (3 mL) was stirred for 2 h at 25° C. under nitrogen atmosphere. The reaction was monitored by LCMS. The resulting mixture was diluted with EtOAc (15 mL). The resulting mixture was washed with 5×15 ml of water. The organic layer was washed with brine (1×15 mL), and dried over anhydrous Na₂SO₄. After filtration, the filtrate was concentrated under reduced pressure. The crude product (150 mg) was purified by Prep-HPLC with the following conditions (Column: Xbridge Phenyl OBD Column, 19*150 mm, 5 m; Mobile Phase A: Water (10 mmol/L NH₄HCO₃, Mobile Phase B: ACN; Flow rate: 60 mL/min mL/min; Gradient: 14% B to 29% B in 10 min; Wave Length: 254 nm/220 nm nm; RT1 (min): 9.35) the pure fraction was concentrated then lyophilized to afford 5-((2-cyclopropyl-6-isopropyl-7-oxo-[1,2,4]triazolo[1,5-a]pyrimidin-4(7H)-yl)methyl)pyridine-2-sulfonamide.

ESI-MS m/z=389.00 [M+H]⁺; Calculated: 388.1.

¹H NMR (400 MHZ, DMSO-d₆) δ 8.79 (d, J=2.2 Hz, 1H), 8.09-8.00 (m, 2H), 7.90 (d, J=8.1 Hz, 1H), 7.44 (s, 2H), 5.43 (s, 2H), 2.98 (p, J=6.9 Hz, 1H), 2.08-2.00 (m, 1H), 1.19 (d, J=6.9 Hz, 6H), 1.00 (dt, J=8.2, 3.2 Hz, 2H), 0.99-0.85 (m, 2H).

Total proton count from structure: 20.

Total proton count from spectrum: 20.

### Compound 14: (1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-sulfonamide

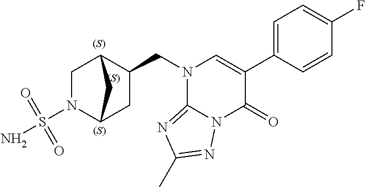

### Step 1: Methyl 2-(4-fluorophenyl)-3-oxopropanoate

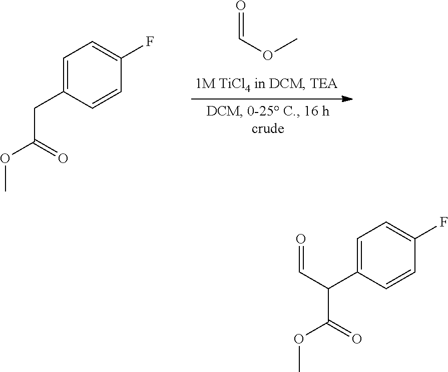

To a stirred solution of methyl 2-(4-fluorophenyl)acetate (10.00 g, 59.5 mmol, 1.0 equiv) and methyl formate (10.71 g, 178.4 mmol, 3.0 equiv) in DCM (200 mL) were added 1M TiCl₄ in DCM (119 mL, 118.9 mmol, 2.0 equiv) and TEA (14.44 g, 142.7 mmol, 2.4 equiv) dropwise at 0° C. under nitrogen atmosphere. The resulting mixture was stirred for an additional 16 h at 25° C. The desired product could be detected by LCMS. The resulting mixture was poured into water (500 mL). The resulting mixture was extracted with EtOAc (3×500 mL). The combined organic layers were washed with brine (1 L), and dried over anhydrous Na₂SO₄. After filtration, the filtrate was concentrated under reduced pressure to afford methyl 2-(4-fluorophenyl)-3-oxopropanoate (crude).

ESI-MS m/z=197.05 [M+H]⁺; Calculated: 196.1.

### Step 2: 6-(4-Fluorophenyl)-2-methyl-[1,2,4]triazolo[1,5-a]pyrimidin-7(4H)-one

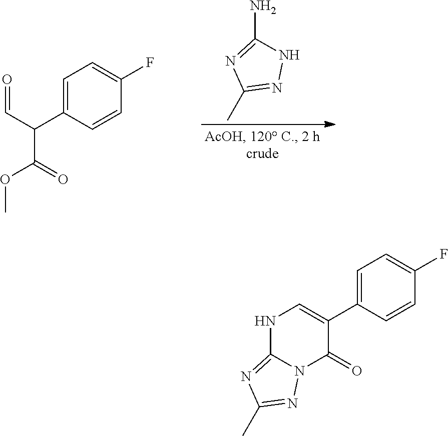

A solution of methyl methyl 2-(4-fluorophenyl)-3-oxopropanoate (2.00 g, 10.2 mmol, 1.0 equiv) and 5-methyl-2H-1,2,4-triazol-3-amine (1.10 g, 11.2 mmol, 1.1 equiv) in AcOH (20 mL) was stirred for 2 h at 120° C. under nitrogen atmosphere. The desired product could be detected by LCMS. The mixture was allowed to cool down to 25° C. The residue was purified by trituration with water (10 mL) to afford 6-(4-fluorophenyl)-2-methyl-[1,2,4]triazolo[1,5-a]pyrimidin-7(4H)-one (crude).

ESI-MS m/z=244.90 [M+H]⁺; Calculated: 244.1.

### Step 3: Tert-butyl(1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-carboxylate

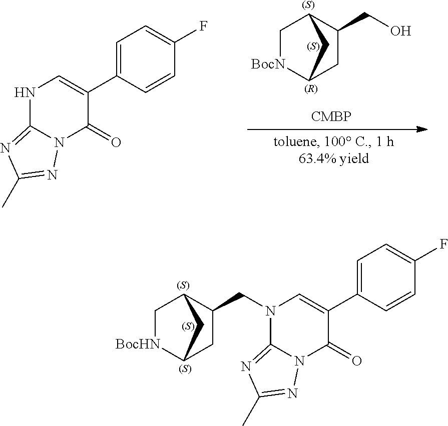

To a stirred solution of 6-(4-fluorophenyl)-2-methyl-[1,2,4]triazolo[1,5-a]pyrimidin-7(4H)-one (355 mg, 1.45 mmol, 1.1 equiv) and tert-butyl(1R,4S,5S)-5-(hydroxymethyl)-2-azabicyclo[2.2.1]heptane-2-carboxylate (300 mg, 1.32 mmol, 1.0 equiv) in Toluene (6 mL) was added 2-(tributyl-lambda5-phosphanylidene) acetonitrile (1.59 g, 6.60 mmol, 5.0 equiv) at 25° C. The resulting mixture was stirred for 1 h at 100° C. under nitrogen atmosphere. The reaction was monitored by LCMS. The desired product could be detected by LCMS. The resulting mixture was concentrated under reduced pressure. The residue was purified by silica gel column chromatography, eluted with PE/EA (40% to 100% gradient in 20 min) to afford tert-butyl(1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-carboxylate.

ESI-MS m/z=454.15 [M+H]⁺; Calculated: 453.2.

¹H NMR (400 MHZ, DMSO-d₆) δ 8.41 (s, 1H), 7.75-7.63 (m, 2H), 7.35-7.19 (m, 2H), 4.26-4.11 (m, 1H), 4.11-3.95 (m, 2H), 3.17-3.06 (m, 1H), 2.84 (d, J=9.6 Hz, 1H), 2.47-2.37 (m, 4H), 2.35 (s, 1H), 1.79-1.62 (m, 2H), 1.60-1.45 (m, 1H), 1.41-1.31 (m, 10H).

¹⁹F NMR (376 MHz, DMSO-d₆) δ −114.67.

### Step 4: 4-(((1R,4S,5S)-2-azabicyclo[2.2.1]heptan-5-yl)methyl)-6-(4-fluorophenyl)-2-methyl-[1,2,4]triazolo[1,5-a]pyrimidin-7(4H)-one HCl salt

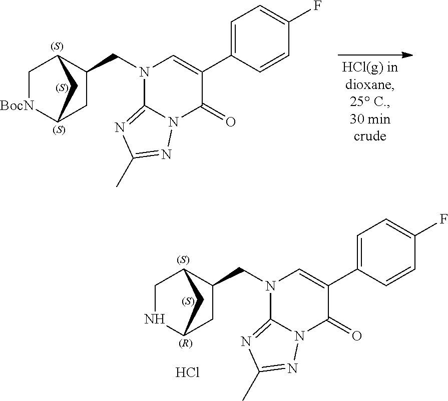

A solution of tert-butyl(1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-carboxylate (200 mg, 0.44 mmol, 1.0 equiv) in HCl (gas) in 1,4-dioxane (2 mL) was stirred for 30 min at 25° C. under nitrogen atmosphere. The desired product could be detected by LCMS. The resulting mixture was concentrated under reduced pressure. This resulted in 4-(((1R,4S,5S)-2-azabicyclo[2.2.1]heptan-5-yl)methyl)-6-(4-fluorophenyl)-2-methyl-[1,2,4]triazolo[1,5-a]pyrimidin-7(4H)-one HCl salt (crude). The crude product mixture was used in the next step directly without further purification.

ESI-MS m/z=354.20 [M+H]⁺; Calculated: 389.1.

### Step 5: (1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-sulfonamide

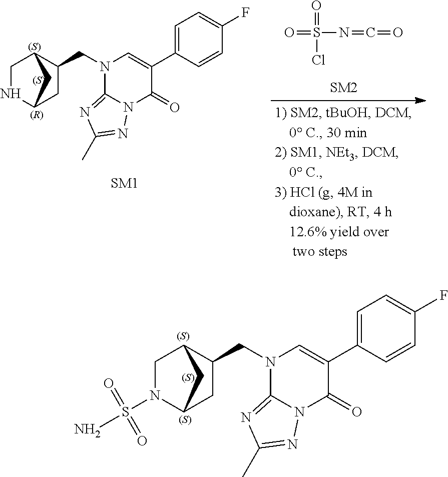

To a stirred solution of chlorosulfonyl isocyanate (320 mg, 2.26 mmol, 4.0 equiv) in DCM (5 mL) was added t-BuOH (378 mg, 5.09 mmol, 9.0 equiv) dropwise at 0° C. under nitrogen atmosphere. The resulting mixture was stirred for 30 min at 0° C. under nitrogen atmosphere. A stirred solution of 4-(((1R,4S,5S)-2-azabicyclo[2.2.1]heptan-5-yl)methyl)-6-(4-fluorophenyl)-2-methyl-[1,2,4]triazolo[1,5-a]pyrimidin-7(4H)-one HCl salt (200 mg, 0.57 mmol, 1.0 equiv) in DCM (5 mL) was treated with Et₃N (573 mg, 5.66 mmol, 10.0 equiv) for 5 min at 25° C. under nitrogen atmosphere followed by the addition of above mixture dropwise at 0° C. The resulting mixture was stirred for 1 h at 0° C. under nitrogen atmosphere. The reaction was monitored by LCMS. The desired product could be detected by LCMS. The resulting mixture was concentrated under reduced pressure. To the above mixture was added HCl (gas) in 1,4-dioxane (10 mL) over 1 min at 25° C. The resulting mixture was stirred for an additional 1 h at 25° C. The reaction was monitored by LCMS. The resulting mixture was concentrated under reduced pressure. The resulting mixture was diluted with water (100 mL). The mixture was neutralized to pH 7 with 1M NaOH(aq.). The resulting mixture was extracted with CH₂Cl₂/IPA (3:1) (3×100 mL). The combined organic layers were washed with brine (1×300 mL), and dried over anhydrous Na₂SO₄. After filtration, the filtrate was concentrated under reduced pressure. The crude product (150 mg) was purified by Prep-HPLC with the following conditions (Column: Kinetex EVO C18 Column, 30*150, 5 μm; Mobile Phase A: 10 mmol/L NH₄HCO₃+0.05% NH₃H₂O), Mobile Phase B: ACN; Flow rate: 60 mL/min; Gradient: 5% B to 5% B in 1 min, 5% B to 12% B in 2 min, 12% to 30% B in 10 min; Wave Length: 254 nm/220 nm; RT1 (min): 8.5), the pure factions were concentrated under vacuum then lyophilized to afford (1S,4S,5S)-5-((6-(4-fluorophenyl)-2-methyl-7-oxo-[1,2,4]triazolo[1,5-a]pyrimidin-4(7H)-yl)methyl)-2-azabicyclo[2.2.1]heptane-2-sulfonamide.

ESI-MS m/z=433.05 [M+H]⁺; Calculated: 432.1.

¹H NMR (400 MHZ, DMSO-d₆) δ 8.43 (s, 1H), 7.76-7.67 (m, 2H), 7.35-7.25 (m, 2H), 6.67 (s, 2H), 4.22-4.11 (m, 1H), 4.05-3.94 (m, 2H), 3.13-3.04 (m, 1H), 2.74 (d, J=9.0 Hz, 1H), 2.48-2.38 (m, 5H), 1.91 (t, J=10.6 Hz, 1H), 1.74-1.54 (m, 2H), 1.35-1.25 (m, 1H).

¹⁹F NMR (376 MHZ, DMSO-d₆) δ −114.64.

Total proton count from structure: 21.

Total proton count from spectrum: 21.

### Compound 15: rel-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide

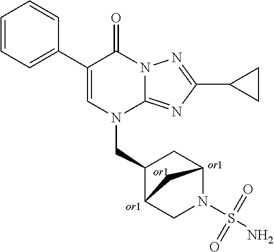

### Step 1: 2-Cyclopropyl-6-phenyl-4H-[1,2,4]triazolo[1,5-a]pyrimidin-7-one

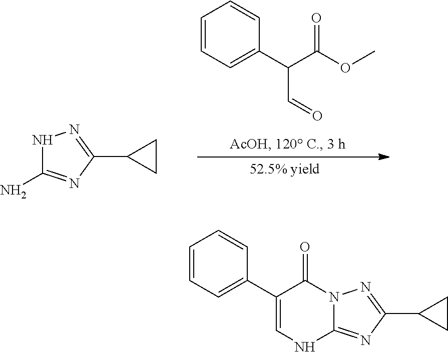 [SMILES: COC(=O)C(C=O)c1ccccc1 ; Nc1nc(C2CC2)n[nH]1 ; O=c1c(-c2ccccc2)c[nH]c2nc(C3CC3)nn12]

To a stirred solution of 5-cyclopropyl-2H-1,2,4-triazol-3-amine (975 mg, 7.85 mmol, 1.4 equiv) in AcOH (10 mL) were added methyl 3-oxo-2-phenylpropanoate (1.00 g, 5.61 mmol, 1.0 equiv) at 25° C. under nitrogen atmosphere. The resulting mixture was stirred for 3 h at 120° C. The mixture was allowed to cool down to 25° C. The reaction was monitored by LCMS. The product was precipitated by the addition of water (50 mL). The precipitated solids were collected by filtration and washed with water (3×6 mL). The solids was dried under reduced pressure to afford 2-cyclopropyl-6-phenyl-4H-[1,2,4]triazolo[1,5-a]pyrimidin-7-one.

ESI-MS m/z=253.1 [M+H]⁺; Calculated: 252.1.

¹H NMR (400 MHZ, DMSO-d₆) δ 13.38 (s, 1H), 8.14 (s, 1H), 7.67-7.62 (m, 2H), 7.46-7.38 (m, 2H), 7.37-7.30 (m, 1H), 2.19-2.03 (m, 1H), 1.09-1.01 (m, 2H), 0.97-0.90 (m, 2H).

### Step 2: Rac-tert-butyl(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-carboxylate

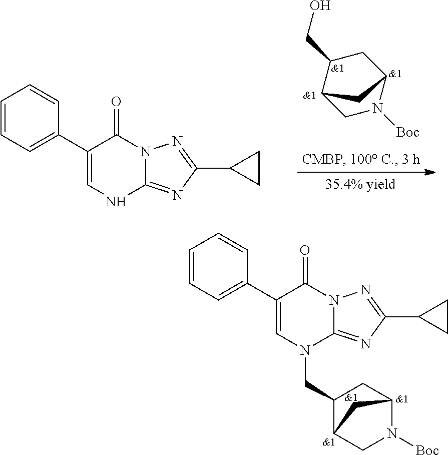 [SMILES: O=c1c(-c2ccccc2)c[nH]c2nc(C3CC3)nn12 ; CC(C)(C)OC(=O)N1C[C@H]2C[C@@H]1C[C@@H]2CO ; CC(C)(C)OC(=O)N1C[C@H]2C[C@@H]1C[C@@H]2Cn1cc(-c2ccccc2)c(=O)n2nc(C3CC3)nc12]

To a stirred solution of 2-cyclopropyl-6-phenyl-4H-[1,2,4]triazolo[1,5-a]pyrimidin-7-one (250 mg, 1.40 mmol, 1.0 equiv) and rac-tert-butyl(1R,4S,5S)-5-(hydroxymethyl)-2-azabicyclo[2.2.1]heptane-2-carboxylate (430 mg, 1.82 mmol, 1.3 equiv) in toluene (3 mL) were added CMBP (3.51 g, 14.0 mmol, 10.0 equiv) at 25° C. under nitrogen atmosphere. The resulting mixture was stirred for 3 h at 100° C. The mixture was allowed to cool down to 25° C. The reaction was monitored by LCMS. The residue was purified by silica gel column chromatography, eluted with PE/EA (1:2) to afford rac-tert-butyl(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-carboxylate.

ESI-MS m/z=462.0 [M+H]⁺; Calculated: 461.2.

### Step 3: Rac-4-(((1R,4S,5S)-2-azabicyclo[2.2.1]heptan-5-yl)methyl)-2-cyclopropyl-6-phenyl-[1,2,4]triazolo[1,5-a]pyrimidin-7(4H)-one hydrochloride

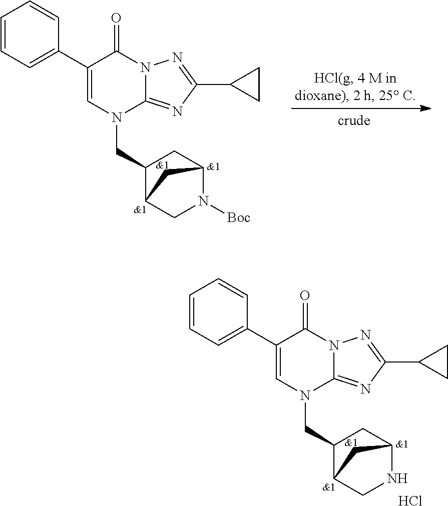 [SMILES: CC(C)(C)OC(=O)N1C[C@H]2C[C@@H]1C[C@@H]2Cn1cc(-c2ccccc2)c(=O)n2nc(C3CC3)nc12 ; O=c1c(-c2ccccc2)cn(C[C@H]2C[C@H]3C[C@@H]2CN3)c2nc(C3CC3)nn12 ; Cl]

A solution of rac-tert-butyl(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-carboxylate (200 mg, 0.43 mmol, 1.0 equiv) in HCl (g, 4 M in 1,4-dioxane, 5 mL) was stirred for 2 h at 25° under nitrogen atmosphere. The reaction was monitored by LCMS. The resulting mixture was concentrated under reduced pressure. The residue was purified by trituration with Et₂O (10 mL). The precipitated solids were collected by filtration and washed with Et₂O (3×5 mL). The resulting mixture was concentrated under reduced pressure. This resulted in rac-4-(((1R,4S,5S)-2-azabicyclo[2.2.1]heptan-5-yl)methyl)-2-cyclopropyl-6-phenyl-[1,2,4]triazolo[1,5-a]pyrimidin-7(4H)-one hydrochloride (crude).

ESI-MS m/z=362.0 [M−HCl+H]⁺; Calculated: 397.2.

### Step 4: Rac-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide

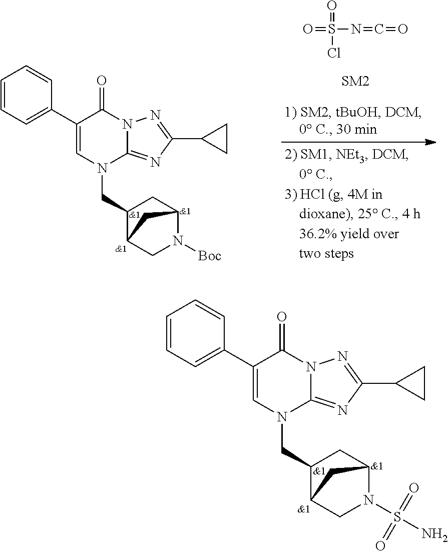 [SMILES: O=C=NS(=O)(=O)Cl ; CC(C)(C)OC(=O)N1C[C@H]2C[C@@H]1C[C@@H]2Cn1cc(-c2ccccc2)c(=O)n2nc(C3CC3)nc12 ; NS(=O)(=O)N1C[C@H]2C[C@@H]1C[C@@H]2Cn1cc(-c2ccccc2)c(=O)n2nc(C3CC3)nc12]

To a stirred solution of chlorosulfonyl isocyanate (148 mg, 1.05 mmol, 2.0 equiv) in DCM (3 mL) was added t-BuOH (77 mg, 1.05 mmol, 2.0 equiv) at 0° C. under nitrogen atmosphere. A solution of rac-4-(((1R,4S,5S)-2-azabicyclo[2.2.1]heptan-5-yl)methyl)-2-cyclopropyl-6-phenyl-[1,2,4]triazolo[1,5-a]pyrimidin-7(4H)-one hydrochloride (190 mg, 0.52 mmol, 1.0 equiv) in DCM (3 mL) was treated with Et₃N (159 mg, 1.57 mmol, 3.0 equiv) for 5 min at room temperature under nitrogen atmosphere followed by the addition of above mixture dropwise at 0° C. The resulting mixture was stirred for 2 h at 25° C. under nitrogen atmosphere. The reaction was monitored by LCMS. To the above mixture was added HCl (g, 4M in 1,4-dioxane, 6 mL) at 25° C. The resulting mixture was stirred 4 h at 25° C. The reaction was monitored by LCMS. The resulting mixture was concentrated under reduced pressure. The residue was dissolved in water (20 mL). The resulting mixture was extracted with EtOAc (2×20 mL). The combined organic layers were washed with brine (1×20 mL), and dried over anhydrous Na₂SO₄. After filtration, the filtrate was concentrated under reduced pressure. The residue was purified by reversed-phase flash chromatography with the following conditions: Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min mL/min; Gradient: 13% B to 23% B in 10 min; Wave Length: 254 nm/220 nm nm; RT1 (10.75 min) to afford rac-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide.

ESI-MS m/z=441.1 [M+H]⁺; Calculated: 440.2.

### Step 5: Rel-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide

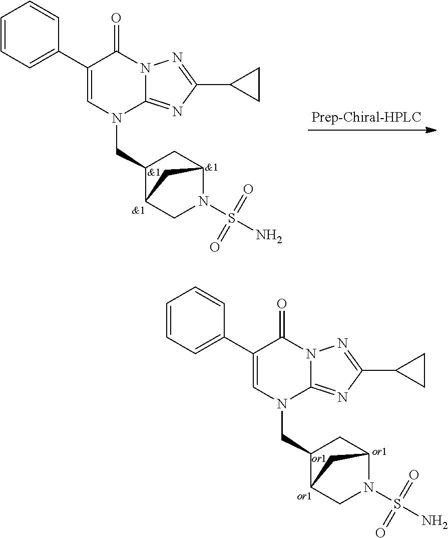

The rac-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide (140 mg) was isolated by Prep-Chiral-HPLC with the following conditions: Column: CHIRAL ART Cellulose-SB, 2*25 cm, 5 μm; Mobile Phase A: MtBE (10 mM NH₃-MeOH), Mobile Phase B: EtOH; Flow rate: 20 mL/min; Gradient: isocratic 30; Wave Length: 224/300 nm; RT1 (min): 8.2; RT2 (min): 10.6; Sample Solvent: TFE:DCM=2:1; Injection Volume: 1.0 mL; Number Of Runs: 16, the pure fraction was concentrated under vacuum and was lyophilized to afford rel-(1R,4R,5R)-5-((2-cyclopropyl-7-oxo-6-phenyl-[1,2,4]triazolo[1,5-a]pyrimidin-4(7H)-yl)methyl)-2-azabicyclo[2.2.1]heptane-2-sulfonamide.

ESI-MS m/z=441.15 [M+H]⁺; Calculated: 440.2.

¹H NMR (400 MHZ, DMSO-d₆) δ 8.40 (s, 1H), 7.73-7.60 (m, 2H), 7.45 (t, J=7.6 Hz, 2H), 7.40-7.29 (m, 1H), 6.67 (s, 2H), 4.13 (dd, J=13.8, 9.3 Hz, 1H), 4.04-3.90 (m, 2H), 3.08 (dd, J=9.1, 3.6 Hz, 1H), 2.73 (d, J=9.1 Hz, 1H), 2.46-2.33 (m, 2H), 2.16-2.08 (m, 1H), 1.90 (dd, J=12.5, 9.0 Hz, 1H), 1.68 (d, J=10.3 Hz, 1H), 1.61 (d, J=10.4 Hz, 1H), 1.35-1.25 (m, 1H), 1.09-1.02 (m, 2H), 0.98-0.92 (m, 2H).

Total proton counts from structure: 24.

Total proton counts from spectrum: 24.

### Compound 16: 5-((6-isopropyl-2-methyl-7-oxopyrazolo[1,5-a]pyrimidin-4(7H)-yl)methyl)pyridine-2-sulfonamide

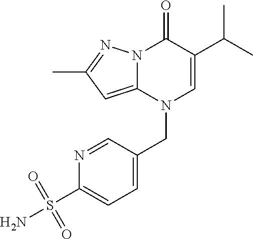 [SMILES: Cc1cc2n(Cc3ccc(S(N)(=O)=O)nc3)cc(C(C)C)c(=O)n2n1]

### Step 1: 5-((6-Isopropyl-2-methyl-7-oxopyrazolo[1,5-a]pyrimidin-4(7H)-yl)methyl)pyridine-2-sulfonamide

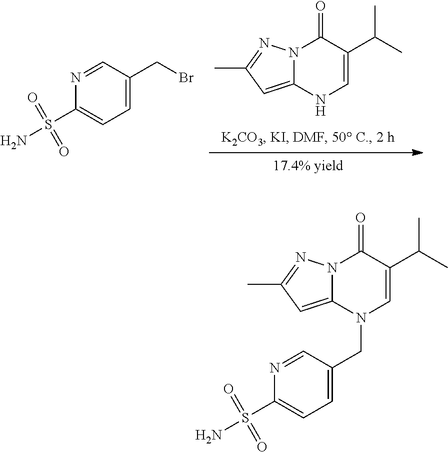

A solution of 5-(bromomethyl)pyridine-2-sulfonamide (400 mg, 1.59 mmol, 1.0 equiv) and 6-isopropyl-2-methyl-4H-pyrazolo[1,5-a]pyrimidin-7-one (243 mg, 1.27 mmol, 0.8 equiv), K₂CO₃ (660 mg, 4.77 mmol, 3.0 equiv), KI (264 mg, 1.59 mmol, 1.0 equiv) in DMF (4 mL) was stirred for 2 h at 50° C. under nitrogen atmosphere. The reaction was monitored by LCMS. The mixture was allowed to cool down to room temperature. The resulting mixture was diluted with water (50 mL). The mixture neutralized to pH 4 with conc. HCl. The resulting mixture was extracted with EtOAc (3×50 mL), and dried over anhydrous Na₂SO₄. After filtration, the filtrate was concentrated under reduced pressure. The crude product (150 mg) was purified by Prep-HPLC with the following conditions (Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 8% B to 24% B in 8 min; Wave Length: 254 nm/220 nm nm; RT1 (min): 9.53), the pure fraction was concentrated then lyophilized to afford 5-((6-isopropyl-2-methyl-7-oxopyrazolo[1,5-a]pyrimidin-4(7H)-yl)methyl)pyridine-2-sulfonamide.

ESI-MS m/z=362.10. [M+H]⁺; Calculated: 361.1.

¹H NMR (400 MHZ, DMSO-d₆) δ 8.76 (d, J=2.1 Hz, 1H), 8.01 (s, 1H), 7.98-7.87 (m, 2H), 7.47 (s, 2H), 6.10 (s, 1H), 5.39 (s, 2H), 3.01 (h, J=6.8 Hz, 1H), 2.24 (s, 3H), 1.21 (d, J=6.9 Hz, 6H).

Total proton count from structure: 19.

Total proton count from spectrum: 19

### Compound 17: 3-fluoro-4-((6-isopropyl-2-methyl-7-oxopyrazolo[1,5-a]pyrimidin-4(7H)-yl)methyl)benzenesulfonamide

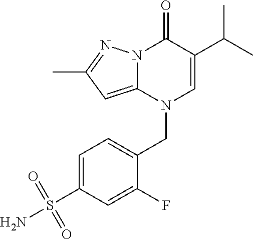 [SMILES: Cc1cc2n(Cc3ccc(S(N)(=O)=O)cc3F)cc(C(C)C)c(=O)n2n1]

### Step 1: 3-Fluoro-4-methylbenzenediazonium; tetrafluoroborate

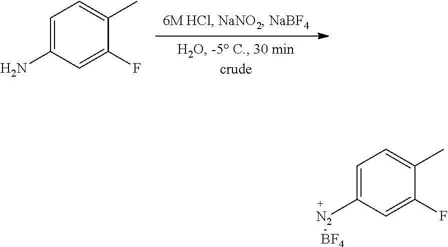

To a stirred solution of 3-fluoro-4-methylaniline (6.00 g, 47.9 mmol, 1.0 equiv) in HCl (6M) (33 mL) was added NaNO₂ (3.47 g, 50.3 mmol, 1.1 equiv) dropwise at −5° C. under nitrogen atmosphere. To the above mixture was added sodium fluoroborate (2.11 g, 19.2 mmol, 1.2 equiv) in H₂O (1.5 mL) dropwise at −5° C. The resulting mixture was stirred for an additional 0.5 h at −5° C. The desired product could be detected by LCMS. The precipitated solids were collected by filtration to afford 3-fluoro-4-methylbenzenediazonium tetrafluoroborate (crude).

ESI-MS m/z=137.15. [M-BF₄]⁺; Calculated: 224.1.

### Step 2: 3-Fluoro-4-methylbenzenesulfonyl chloride

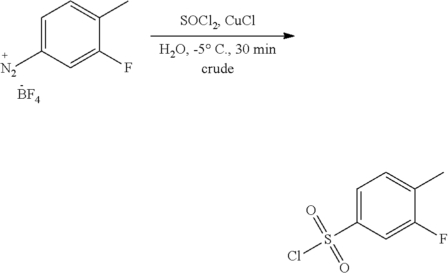

To a stirred solution of H₂O (60 mL) was added SOCl₂ (22.82 g, 191.8 mmol, 4.0 equiv) dropwise at 0° C. under nitrogen atmosphere. To the above mixture was added CuCl (950 mg, 9.59 mmol, 0.20 equiv) in portions at 0° C. To the above mixture was added 3-fluoro-4-methylbenzenediazonium tetrafluoroborate (10.74 g, 48.0 mmol, 1.0 equiv) in portions at −5° C. The resulting mixture was stirred for an additional 0.5 h at −5° C. The desired product could be detected by LCMS. The resulting mixture was diluted with water (50 mL). The resulting mixture was extracted with EtOAc (3×150 mL). The combined organic layers were washed with brine (200 mL), and dried over anhydrous Na₂SO₄. After to filtration, the filtrate was concentrated under reduced pressure afford 3-fluoro-4-methylbenzenesulfonyl chloride (crude).

### Step 3: 3-Fluoro-4-methylbenzenesulfonamide

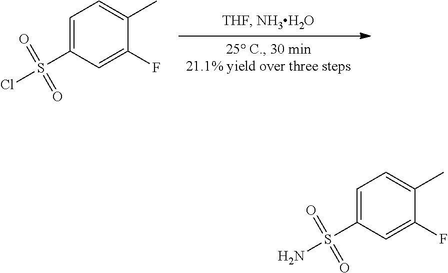

To a stirred solution of 3-fluoro-4-methylbenzenesulfonyl chloride (8.00 g, 38.3 mmol, 1.0 equiv) in THF (100 mL) was added NH₃·H₂O (20 mL) dropwise at 0° C. under nitrogen atmosphere. The resulting mixture was stirred for 0.5 h at 0° C. under nitrogen atmosphere. The desired product could be detected by LCMS. The resulting mixture was concentrated under reduced pressure. The resulting mixture was concentrated under vacuum. The residue was purified by silica gel column chromatography, eluted with PE/THF (15% to 50% gradient in 20 min) to afford 3-fluoro-4-methylbenzenesulfonamide.

ESI-MS m/z=187.90. [M−H]⁺; Calculated: 189.1.

¹H NMR (400 MHZ, DMSO-d₆) δ 7.59-7.47 (m, 3H), 7.42 (s, 2H), 2.30 (d, J=2.1 Hz, 3H).

¹⁹F NMR (376 MHZ, DMSO-d₆) δ −115.38.

### Step 4: 4-(Bromomethyl)-3-fluorobenzenesulfonamide

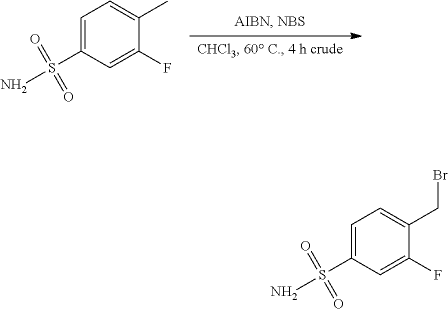

To a stirred solution of 3-fluoro-4-methylbenzenesulfonamide (500 mg, 2.64 mmol, 1.0 equiv) in CHCl3 (15 mL) were added AIBN (43 mg, 0.26 mmol, 0.10 equiv) and NBS (706 mg, 3.96 mmol, 1.5 equiv) in portions at 25° C. under nitrogen atmosphere. The resulting mixture was stirred for 16 h at 60° C. under nitrogen atmosphere. The mixture was allowed to cool down to 25° C. The resulting mixture was concentrated under reduced pressure. The residue was purified by silica gel column chromatography, eluted with PE/EA (10% to 40% gradient in 20 min) to afford 4-(bromomethyl)-3-fluorobenzenesulfonamide (crude).

ESI-MS m/z=265.70&267.80 [M−H]⁻; Calculated: 266.9&268.9.

### Step 5: 3-Fluoro-4-((6-isopropyl-2-methyl-7-oxopyrazolo[1,5-a]pyrimidin-4(7H)-yl)methyl)benzenesulfonamide

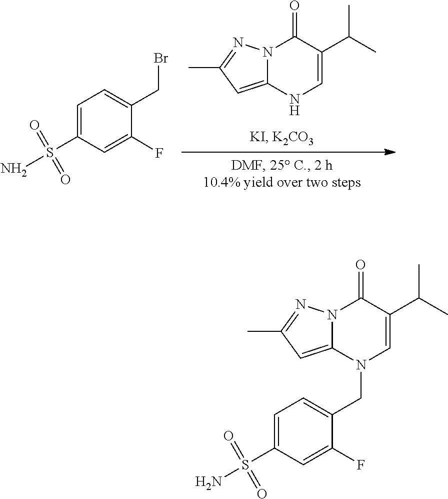

To a stirred solution of 4-(bromomethyl)-3-fluorobenzenesulfonamide (336 mg, 1.26 mmol, 2.0 equiv) and 6-isopropyl-2-methyl-4H-pyrazolo[1,5-a]pyrimidin-7-one (120 mg, 0.63 mmol, 1.0 equiv) in DMF (3 mL) were added KI (104 mg, 0.63 mmol, 1.0 equiv) and K₂CO₃ (173 mg, 1.26 mmol, 2.0 equiv) in portions at 25° C. under nitrogen atmosphere. The resulting mixture was stirred for 1 h at 25° C. under nitrogen atmosphere. The desired product could be detected by LCMS. The resulting mixture was diluted with water (50 mL). The resulting mixture was extracted with EtOAc (3×50 mL). The combined organic layers were washed with brine (3×100 mL), and dried over anhydrous Na₂SO₄. After filtration, the filtrate was concentrated under reduced pressure. The crude product (200 mg) was purified by Prep-HPLC with the following conditions (Column: XBridge Prep Phenyl OBD Column 19*250 mm, 5 m; Mobile Phase A: 10 mmol/L NH₄HCO₃+0.05% NH₃H₂O, Mobile Phase B: ACN; Flow rate: 60 mL/min; Gradient: 5% B to 5% B in 1 min, 5% B to 36% B in 2 min, 36% to 50% B in 10 min; Wave Length: 254 nm/220 nm; RT1 (min): 9.25), the pure fractions were concentrated and then lyophilized to afford 3-fluoro-4-({6-isopropyl-2-methyl-7-oxopyrazolo[1,5-a]pyrimidin-4-yl}methyl)benzenesulfonamide.

ESI-MS m/z=379.15. [M+H]⁺; Calculated: 378.1.

¹H NMR (400 MHZ, DMSO-d₆) δ 7.93 (s, 1H), 7.71-7.61 (m, 2H), 7.60-7.43 (m, 3H), 6.02 (s, 1H), 5.43 (s, 2H), 3.08-2.98 (m, 1H), 2.25 (s, 3H), 1.20 (d, J=6.9 Hz, 6H).

¹⁹F NMR (376 MHZ, DMSO-d₆) δ −114.502.

Total proton count from structure: 19.

Total proton count from spectrum: 19.

### Compound 18: 2-(difluoromethoxy)-4-((6-isopropyl-2-methyl-7-oxopyrazolo[1,5-a]pyrimidin-4(7H)-yl)methyl)benzenesulfonamide

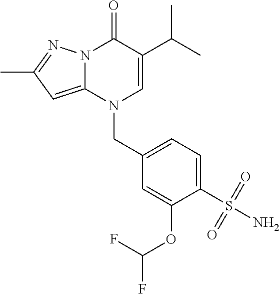 [SMILES: Cc1cc2n(Cc3ccc(S(N)(=O)=O)c(OC(F)F)c3)cc(C(C)C)c(=O)n2n1]

### Step 1: 2-(Difluoromethoxy)-4-((6-isopropyl-2-methyl-7-oxopyrazolo[1,5-a]pyrimidin-4(7H)-yl)methyl)benzenesulfonamide

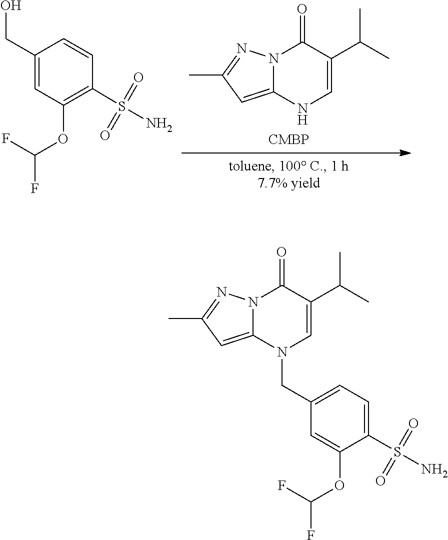 [SMILES: NS(=O)(=O)c1ccc(CO)cc1OC(F)F ; Cc1cc2[nH]cc(C(C)C)c(=O)n2n1 ; Cc1cc2n(Cc3ccc(S(N)(=O)=O)c(OC(F)F)c3)cc(C(C)C)c(=O)n2n1]

To a stirred solution of 2-(difluoromethoxy)-4-(hydroxymethyl)benzenesulfonamide (200 mg, 0.79 mmol, 1.0 equiv) and 6-isopropyl-2-methyl-4H-pyrazolo[1,5-a]pyrimidin-7-one (166 mg, 0.87 mmol, 1.1 equiv) in Toluene (5 mL) was added 2-(tributyl-lambda5-phosphanylidene) acetonitrile (381 mg, 1.58 mmol, 2.0 equiv) at 25° C. The resulting mixture was stirred for 1 h at 100° C. under nitrogen atmosphere. The desired product could be detected by LCMS. The mixture was allowed to cool down to 25° C. The resulting mixture was concentrated under reduced pressure. The residue was purified by reversed-phase flash chromatography with the following conditions: column: C18; mobile phase: MeCN in Water (0.1% NH₃·H₂O), 25% to 50% gradient in 20 min; detector: UV 254 nm to afford the crude product. The crude product (180 mg) was purified by Prep-HPLC with the following conditions (Column: YMC-Actus Triart C18 EXRS 30*150 mm, 5 μm; Mobile Phase A: Water (10 mmol/L NH₄HCO₃), Mobile Phase B: MEOH; Flow rate: 60 mL/min mL/min; Gradient: 33% B to 49% B in 8 min; Wave Length: 254 nm/220 nm nm; RT1 (min): 9.25), the pure fraction was concentrated then lyophilized to afford 2-(difluoromethoxy)-4-((6-isopropyl-2-methyl-7-oxopyrazolo[1,5-a]pyrimidin-4(7H)-yl)methyl)benzenesulfonamide.

ESI-MS m/z=427.10. [M+H]⁺; Calculated MW: 426.1.

¹H NMR (400 MHZ, DMSO-d₆) δ 7.98 (d, J=1.4 Hz, 1H), 7.86-7.80 (m, 1H), 7.45 (br, 2H), 7.40-7.00 (m, 3H), 6.02 (s, 1H), 5.34 (s, 2H), 3.09-2.95 (m, 1H), 2.24 (s, 3H), 1.20 (d, J=6.9 Hz, 6H).

¹⁹F NMR (282 MHZ, DMSO) δ −82.274.

Total proton count from structure: 20.

Total proton count from spectrum: 20.

### Compound 19: 4-((2-methyl-7-oxo-6-phenylpyrazolo[1,5-a]pyrimidin-4(7H)-yl)methyl)benzenesulfonamide

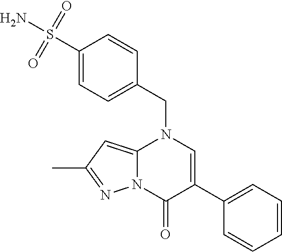 [SMILES: Cc1cc2n(Cc3ccc(S(N)(=O)=O)cc3)cc(-c3ccccc3)c(=O)n2n1]

### Step 1: 2-Methyl-6-phenylpyrazolo[1,5-a]pyrimidin-7(4H)-one

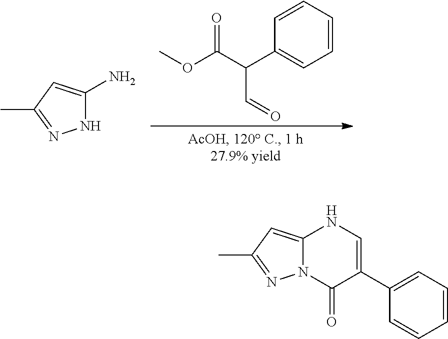

To a stirred solution of 5-methyl-2H-pyrazol-3-amine (500 mg, 5.15 mmol, 1.0 equiv) in AcOH (8 mL) was added methyl 3-oxo-2-phenylpropanoate (917 mg, 5.15 mmol, 1.0 equiv) in portions at 25° C. under nitrogen atmosphere. The resulting mixture was stirred for 1 h at 120° C. under nitrogen atmosphere. The desired product could be detected by LCMS. The mixture was allowed to cool down to 25° C. The residue was purified by trituration with water (50 mL). The precipitated solids were collected by filtration and washed with water (3×5 mL). The resulting mixture was concentrated under vacuum. This resulted in 2-methyl-6-phenyl-4H-pyrazolo[1,5-a]pyrimidin-7-one.

ESI-MS m/z=226.10. [M+H]⁺; Calculated: 225.1.

¹H NMR (400 MHz, DMSO-d₆) δ12.50 (s, 1H), 8.06 (s, 1H), 7.65 (d, J=7.6 Hz, 2H), 7.45-7.36 (m, 2H), 7.36-7.25 (m, 1H), 6.02 (s, 1H), 2.32 (s, 3H).

### Step 2: 4-((2-Methyl-7-oxo-6-phenylpyrazolo[1,5-a]pyrimidin-4(7H)-yl)methyl)benzenesulfonamide

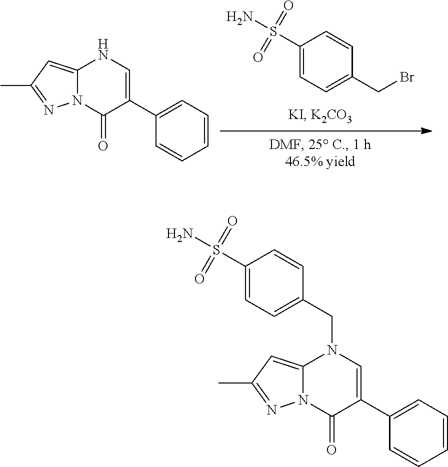 [SMILES: Cc1cc2[nH]cc(-c3ccccc3)c(=O)n2n1 ; NS(=O)(=O)c1ccc(CBr)cc1 ; Cc1cc2n(Cc3ccc(S(N)(=O)=O)cc3)cc(-c3ccccc3)c(=O)n2n1]

To a stirred solution of 2-methyl-6-phenyl-4H-pyrazolo[1,5-a]pyrimidin-7-one (120 mg, 0.53 mmol, 1.0 equiv) and 4-(bromomethyl)benzenesulfonamide (200 mg, 0.80 mmol, 1.5 equiv) in DMF (2 mL) were added KI (88 mg, 0.53 mmol, 1.0 equiv) and K₂CO₃ (147 mg, 1.07 mmol, 2.0 equiv) in portions at 25° C. under nitrogen atmosphere. The resulting mixture was stirred for 1 h at 25° C. under nitrogen atmosphere. The desired product could be detected by LCMS. The resulting mixture was diluted with water (30 mL). The resulting mixture was extracted with EtOAc (3×30 mL). The combined organic layers were washed with brine (50 mL), and dried over anhydrous Na₂SO₄. After filtration, the filtrate was concentrated under reduced pressure. The crude product (200 mg) was purified by Prep-HPLC with the following conditions (Column: Xbridge Phenyl OBD Column, 19*150 mm, 5 m; Mobile Phase A: water (10 mmol/L NH₄HCO₃, Mobile Phase B: ACN; Flow rate: 60 mL/min; Gradient: 20% B to 35% B in 10 min; Wave Length: 254 nm/220 nm; RT1 (min): 9.18), the pure fractions were concentrated and then lyophilized to afford 4-((2-methyl-7-oxo-6-phenylpyrazolo[1,5-a]pyrimidin-4(7H)-yl)methyl)benzenesulfonamide.

ESI-MS m/z=395.05. [M+H]⁺; Calculated: 394.1.

¹H NMR (400 MHZ, DMSO-d₆) δ 8.36 (s, 1H), 7.83-7.70 (m, 2H), 7.61 (d, J=7.6 Hz, 2H), 7.52 (d, J=8.1 Hz, 2H), 7.37-7.27 (m, 4H), 7.26-7.20 (m, 1H), 6.02 (s, 1H), 5.34 (s, 2H), 2.18 (s, 3H).

Total proton count from structure: 18.

Total proton count from spectrum: 18.

### Compound 20: 2-cyclopropyl-6-isopropyl-4-(4-(S-methylsulfonimidoyl)-3-(trifluoromethyl)benzyl) pyrazolo[1,5-a]pyrimidin-7(4H)-one

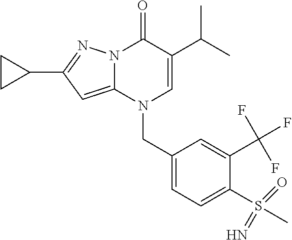 [SMILES: CC(C)c1cn(Cc2ccc(S(C)(=N)=O)c(C(F)(F)F)c2)c2cc(C3CC3)nn2c1=O]

### Step 1: 2-Cyclopropyl-6-isopropyl-4-(4-(S-methylsulfonimidoyl)-3-(trifluoromethyl)benzyl) pyrazolo[1,5-a]pyrimidin-7(4H)-one

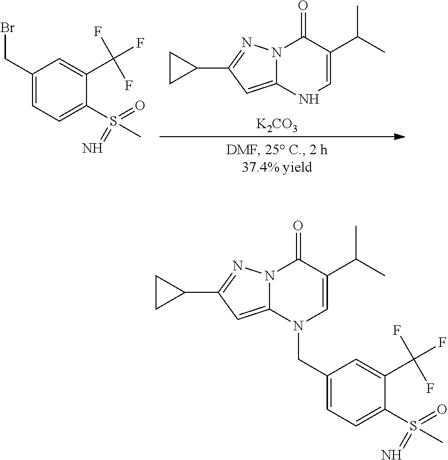

To a stirred mixture of [4-(bromomethyl)-2-(trifluoromethyl)phenyl](imino)methyl-lambda6-sulfanone (500 mg, 1.58 mmol, 1.0 equiv) and 2-cyclopropyl-6-isopropyl-4H-pyrazolo[1,5-a]pyrimidin-7-one (378 mg, 1.74 mmol, 1.1 equiv) in DMF (5 mL) were added K₂CO₃ (437 mg, 3.16 mmol, 2.0 equiv) at 25° C. under nitrogen atmosphere. The resulting mixture was stirred for 2 h at 25° C. under nitrogen atmosphere. The desired product could be detected by LCMS. The resulting mixture was diluted with EtOAc (80 mL). The residue was washed with water (3×30 mL). The resulting mixture was concentrated under reduced pressure. The crude product (300 mg) was purified by Prep-HPLC with the following conditions (Column: Sunfire prep C18 column 30*150 mm, 5 m; Mobile Phase A: Water (0.1% FA), Mobile Phase B: ACN; Flow rate: 60 mL/min mL/min; Gradient: 18% B to 38% B in 9 min; Wave Length: 254 nm/220 nm nm; RT1 (min): 11.43), the pure fraction was concentrated under reduced pressure and then lyophilized to afford 2-cyclopropyl-6-isopropyl-4-(4-(S-methyl-sulfonimidoyl)-3-(trifluoromethyl)benzyl) pyrazolo[1,5-a]pyrimidin-7(4H)-one.

ESI-MS m/z=453.10 [M+H]⁺; Calculated: 452.1.

¹H NMR (400 MHZ, DMSO-d₆) δ 8.24 (d, J=8.2 Hz, 1H), 8.10-7.90 (m, 2H), 7.89-7.64 (m, 1H), 6.09 (s, 1H), 5.39 (s, 2H), 4.57 (s, 1H), 3.10 (s, 3H), 3.06-2.94 (m, 1H), 2.03-1.86 (m, 1H), 1.19 (d, J=6.9 Hz, 3H), 1.19 (d, J=6.9 Hz, 3H), 1.02-0.85 (m, 2H), 0.80-0.66 (m, 2H).

¹⁹F NMR (376 MHZ, DMSO-d₆) δ −55.03.

Total proton count from structure: 23.

Total proton count from spectrum: 23.

### Compound 21: 4-((2-cyclopropyl-6-isopropyl-7-oxo-[1,2,4]triazolo[1,5-a]pyrimidin-4(7H)-yl)methyl)-2-(5-fluoropyridin-2-yl)benzenesulfonamide

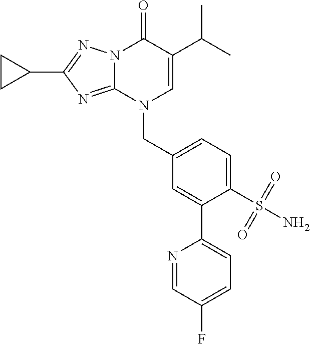 [SMILES: CC(C)c1cn(Cc2ccc(S(N)(=O)=O)c(-c3ccc(F)cn3)c2)c2nc(C3CC3)nn2c1=O]

### Step 1: 3-Bromo-4-sulfamoylbenzoic acid

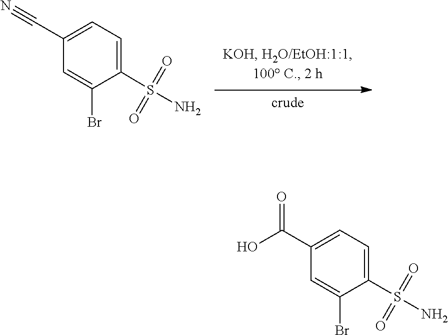 [SMILES: N#Cc1ccc(S(N)(=O)=O)c(Br)c1 ; NS(=O)(=O)c1ccc(C(=O)O)cc1Br]

To a stirred solution of bis(2-bromo-4-cyanobenzenesulfonamide) (4.00 g, 7.66 mmol, 1.0 equiv) and KOH (1.72 g, 30.6 mmol, 4.0 equiv) in H₂O (20 mL) was added EtOH (20 mL) at 25° C. The resulting mixture was stirred for 2 h at 100° C. under nitrogen atmosphere. The desired product could be detected by LCMS. The mixture was allowed to cool down to 25° C. The mixture was acidified to pH 5 with conc. HCl. The resulting mixture was diluted with water (150 mL). The resulting mixture was extracted with EtOAc (3×200 mL). The combined organic layers were washed with brine (100 mL), and dried over anhydrous Na₂SO₄. After filtration, the filtrate was concentrated under reduced pressure. This resulted in bis(3-bromo-4-sulfamoylbenzoic acid) (crude).

ESI-MS m/z=278.0&280.0. [M−H]⁺; Calculated: 278.9&280.9.

¹H NMR (400 MHZ, DMSO-d₆) δ 8.24 (m, 1H), 8.19-8.05 (m, 2H), 7.79 (t, J=4.9 Hz, 2H).

### Step 2: 2-Bromo-4-(hydroxymethyl)benzenesulfonamide

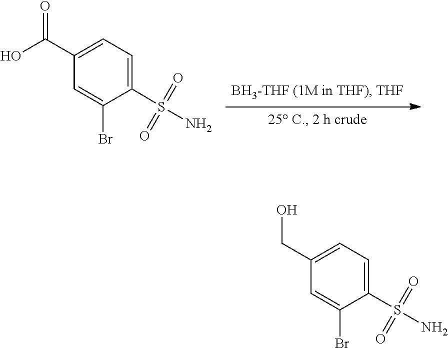 [SMILES: NS(=O)(=O)c1ccc(C(=O)O)cc1Br ; NS(=O)(=O)c1ccc(CO)cc1Br]

To a stirred solution of 3-bromo-4-sulfamoylbenzoic acid (700 mg, 2.50 mmol, 1.0 equiv) in THF (2 mL) were added BH₃-THF (12.5 mL, 12.5 mmol, 5.0 equiv, 1 M in THF) dropwise at 0° C. under nitrogen atmosphere. The resulting mixture was stirred for 2 h at 25° C. under nitrogen atmosphere. The desired product could be detected by LCMS. The reaction was quenched by the addition of Water/Ice (50 mL) at 0° C. The resulting mixture was extracted with EtOAc (3×100 mL). The combined organic layers were washed with brine (100 mL), and dried over anhydrous Na₂SO₄. After filtration, the filtrate was concentrated under reduced pressure. This resulted in 2-bromo-4-(hydroxymethyl)benzenesulfonamide (crude).

ESI-MS m/z=264.0&266.0. [M−H]⁻; Calculated: 264.9&266.9.

### Step 3: 2-Bromo-4-((2-cyclopropyl-6-isopropyl-7-oxo-[1,2,4]triazolo[1,5-a]pyrimidin-4(7H)-yl)methyl)benzenesulfonamide

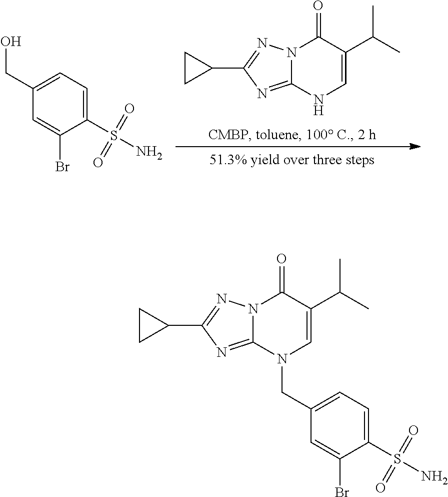 [SMILES: NS(=O)(=O)c1ccc(CO)cc1Br ; CC(C)c1c[nH]c2nc(C3CC3)nn2c1=O ; "CMBP, toluene, 100° C., 2 h" ; CC(C)c1cn(Cc2ccc(S(N)(=O)=O)c(Br)c2)c2nc(C3CC3)nn2c1=O]

To a stirred solution of 2-bromo-4-(hydroxymethyl)benzenesulfonamide (200 mg, 0.75 mmol, 1.0 equiv) and 2-cyclopropyl-6-isopropyl-4H-[1,2,4]triazolo[1,5-a]pyrimidin-7-one (164 mg, 0.75 mmol, 1.0 equiv) in toluene (2 mL) were added CMBP (1.80 g, 7.52 mmol, 10.0 equiv) at 25° C. under nitrogen atmosphere. The resulting mixture was stirred for 2 h at 100° C. under nitrogen atmosphere. The desired product could be detected by LCMS. The mixture was allowed to cool down to 25° C. The resulting mixture was diluted with water (100 mL). The resulting mixture was extracted with EtOAc (3×100 mL). The combined organic layers were washed with water (100 mL), and dried over anhydrous Na₂SO₄. After filtration, the filtrate was concentrated under reduced pressure. The residue was purified by silica gel column chromatography, eluted with PE/EA (1:1) to afford crude product. The crude product was purified by reversed-phase flash chromatography with the following conditions: column: C18; mobile phase: MeCN in Water (0.1% FA), 30% to 50% gradient in 20 min; detector: UV 254 nm. This resulted in 2-bromo-4-((2-cyclopropyl-6-isopropyl-7-oxo-[1,2,4]triazolo[1,5-a]pyrimidin-4(7H)-yl)methyl)benzenesulfonamide.

ESI-MS m/z=466.1&468.1. [M+H]⁺; Calculated: 465.0&467.0.

### Step 4: 4-((2-Cyclopropyl-6-isopropyl-7-oxo-[1,2,4]triazolo[1,5-a]pyrimidin-4(7H)-yl)methyl)-2-(5-fluoropyridin-2-yl)benzenesulfonamide

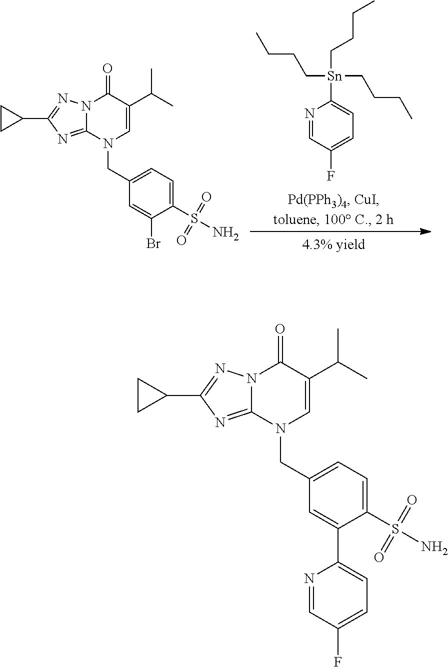 [SMILES: CC(C)c1cn(Cc2ccc(S(N)(=O)=O)c(Br)c2)c2nc(C3CC3)nn2c1=O ; CCC[CH2][Sn]([CH2]CCC)([CH2]CCC)[c]1ccc(F)cn1 ; "Pd(PPh3)4, CuI,toluene, 100° C., 2 h" ; CC(C)c1cn(Cc2ccc(S(N)(=O)=O)c(-c3ccc(F)cn3)c2)c2nc(C3CC3)nn2c1=O]

To a stirred solution of 2-bromo-4-({2-cyclopropyl-6-isopropyl-7-oxo-[1,2,4]triazolo[1,5-a]pyrimidin-4-yl}methyl)benzenesulfonamide (180 mg, 0.40 mmol, 1.0 equiv) and 5-fluoro-2-(tributylstannyl)pyridine (150 mg, 0.40 mmol, 1.0 equiv) in toluene (2 mL) were added Pd(PPh₃)₄ (9 mg, 0.04 mmol, 0.10 equiv) and CuI (74 mg, 0.40 mmol, 1.0 equiv) at 25° C. under argon atmosphere. The resulting mixture was stirred for 2 h at 100° C. The desired product could be detected by LCMS. The mixture was allowed to cool down to 25° C. The resulting mixture was concentrated under vacuum. The residue was purified by reversed-phase flash chromatography with the following conditions: column: C18; mobile phase: MeCN in Water (0.1% NH₃·H₂O), 30% to 50% gradient in 20 min; detector: UV 254 nm to afford crude product. The crude product (100 mg) was purified by Prep-HPLC with the following conditions (Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min mL/min; Gradient: 20% B to 35% B in 10 min; Wave Length: 254 nm/220 nm nm; RT1 (min): 9.12) the pure fraction was concentrated then lyophilized to afford 4-((2-cyclopropyl-6-isopropyl-7-oxo-[1,2,4]triazolo[1,5-a]pyrimidin-4(7H)-yl)methyl)-2-(5-fluoropyridin-2-yl)benzenesulfonamide.

ESI-MS m/z=483.10 [M+H]⁺; Calculated: 482.2.

¹H NMR (400 MHZ, DMSO-d₆) δ 8.61 (s, 1H), 8.46 (d, J=4.8 Hz, 1H), 8.06-7.96 (m, 2H), 7.62 (dd, J=8.2, 1.8 Hz, 1H), 7.48 (d, J=16.0 Hz, 3H), 7.38 (dd, J=6.4, 4.8 Hz, 1H), 5.40 (s, 2H), 3.02-2.96 (m, 1H), 2.06-2.00 (m, 1H), 1.17 (d, J=6.9 Hz, 6H), 1.02-0.95 (m, 2H), 0.90-0.80 (m, 2H).

Total proton count from structure: 23

Total proton count from spectrum: 23

### Compound 22: 4-((2-cyclopropyl-6-isopropyl-7-oxo-[1,2,4]triazolo[1,5-a]pyrimidin-4(7H)-yl)methyl)-2-methylbenzenesulfonamide

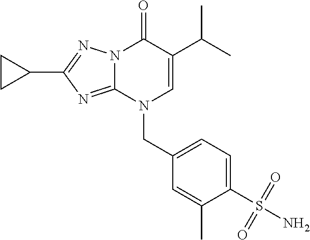 [SMILES: Cc1cc(Cn2cc(C(C)C)c(=O)n3nc(C4CC4)nc23)ccc1S(N)(=O)=O]

### Step 1: 4-(Benzylthio)-3-methylbenzonitrile

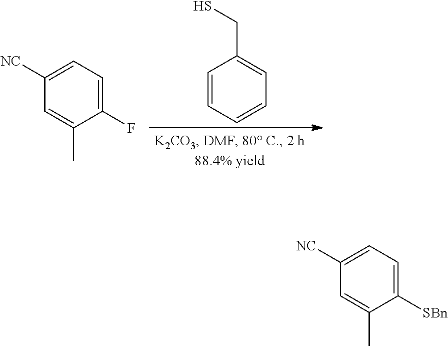

To a stirred solution of 4-fluoro-3-methylbenzonitrile (30.00 g, 222.0 mmol, 1.0 equiv) and benzyl mercaptan (33.10 g, 266.4 mmol, 1.2 equiv) in DMF (30 mL) were added K₂CO₃ (61.36 g, 444.0 mmol, 2.0 equiv) at 25° C. under nitrogen atmosphere. The resulting mixture was stirred for an additional 2 h at 80° C. under nitrogen atmosphere. The desired product could be detected by LCMS. The mixture was allowed to cool down to 25° C. The resulting mixture was diluted with water (100 mL). The resulting mixture was extracted with EtOAc (3×500 mL). The combined organic layers were washed with brine (100 mL), and dried over anhydrous Na₂SO₄. After filtration, the filtrate was concentrated under reduced pressure. The residue was purified by silica gel column chromatography, eluted with PE/EA (6:1) to afford 4-(benzylsulfanyl)-3-methylbenzonitrile.

ESI-MS m/z=240.1 [M+H]⁺; Calculated: 239.1.

¹H NMR (400 MHZ, DMSO-d₆) δ 7.61 (dt, J=5.9, 2.6 Hz, 2H), 7.50-7.42 (m, 3H), 7.38-7.24 (m, 3H), 4.37 (t, J=2.6 Hz, 2H), 2.24 (s, 3H).

### Step 2: 4-Cyano-2-methylbenzenesulfonyl chloride

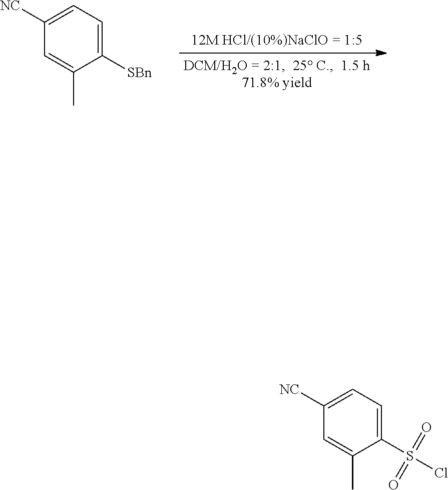

To a stirred solution of 4-(benzylsulfanyl)-8-bromoquinoline (15.00 g, 45.4 mmol, 1.0 equiv) in H₂O (225.00 mL) and DCM (450 mL) were added (12 M) HCl (90 mL) and (10%) NaClO (450 mL) at 25° C. under air atmosphere. The resulting mixture was stirred for an additional 1.5 h at 25° C. under nitrogen atmosphere. The desired product could be detected by LCMS. The mixture was allowed to cool down to 25° C. The resulting mixture was diluted with water (100 mL). The resulting mixture was extracted with CH₂Cl₂ (3×100 mL). The combined organic layers were washed with brine (100 mL), and dried over anhydrous Na₂SO₄. After filtration, the filtrate was concentrated under reduced pressure. This resulted in 4-cyano-2-methylbenzenesulfonyl chloride.

ESI-MS m/z=196.0 [M−H]⁺ as sulfonic acid; Calculated: 214.9.

### Step 3: 4-Cyano-2-methylbenzenesulfonamide

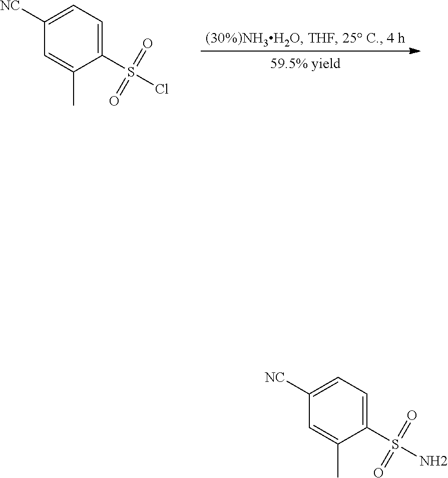

To a stirred solution of 4-cyano-2-methylbenzenesulfonyl chloride (43.00 g, 199.4 mmol, 1.0 equiv) in THF (1720 mL) were added (30%) NH₃·H₂O (330 mL) dropwise at 25° C. The resulting mixture was stirred for an additional 4 h at 25° C. under nitrogen atmosphere. The desired product could be detected by LCMS. The resulting mixture was concentrated under vacuum. The residue was purified by silica gel column chromatography, eluted with PE/EA (3:1) to afford 4-cyano-2-methylbenzenesulfonamide.

ESI-MS m/z=195.0 [M−H]⁻; Calculated: 196.0.

¹H NMR (400 MHZ, DMSO-d₆) δ 8.01-7.85 (m, 3H), 7.71 (br, 2H), 2.62 (s, 3H).

### Step 4: 4-Formyl-2-methylbenzenesulfonamide

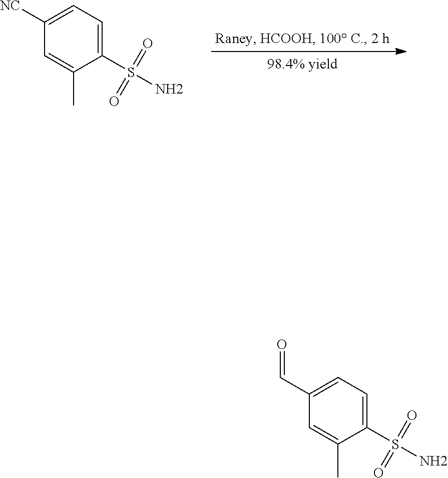

To a stirred solution of 4-cyano-2-methylbenzenesulfonamide (5.00 g, 25.5 mmol, 1.0 equiv) in formic acid (60 mL) were added Raney-Ni (502 mg, 5.86 mmol, 0.23 equiv) dropwise at 25° C. under nitrogen atmosphere. The resulting mixture was stirred for an additional 2 h at 100° C. under nitrogen atmosphere. The desired product could be detected by LCMS. The mixture was allowed to cool down to 25° C. The resulting mixture was filtered, the filter cake was washed with MeOH (3×10 mL). The filtrate was concentrated under reduced pressure. The residue was purified by reversed-phase flash chromatography with the following conditions: column: C18; mobile phase: MeCN in Water (0.1% FA), 20% to 40% gradient in 20 min; detector: UV 254 nm. This resulted in 4-formyl-2-methylbenzenesulfonamide.

ESI-MS m/z=197.0 [M+H]⁺; Calculated: 196.0.

¹H NMR (400 MHZ, DMSO-d₆) δ 10.06 (s, 1H), 8.06 (d, J=8.5 Hz, 1H), 7.91 (dd, J=5.5, 2.1 Hz, 2H), 7.66 (br, 2H), 2.69 (s, 3H).

### Step 5: 4-(Hydroxymethyl)-2-methylbenzenesulfonamide

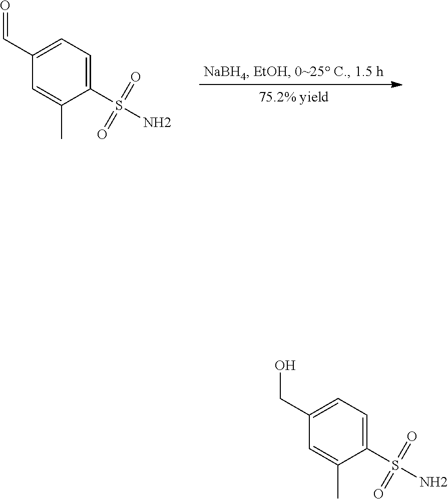

To a stirred solution of 4-formyl-2-methylbenzenesulfonamide (5.00 g, 25.1 mmol, 1.0 equiv) in THF (500 mL) were added NaBH₄ (1.89 g, 50.2 mmol, 2.0 equiv) at 25° C. The resulting mixture was stirred for an additional 1.5 h at 25° C. under nitrogen atmosphere. The reaction was quenched with Water at 0° C. The mixture was acidified to pH 3 with conc. HCl. The resulting mixture was extracted with CHCl3/IPA (5:1) (3×100 mL). The combined organic layers were washed with brine (100 mL), and dried over anhydrous Na₂SO₄. After filtration, the filtrate was concentrated under reduced pressure. This resulted in 4-(hydroxymethyl)-2-methylbenzenesulfonamide.

ESI-MS m/z=200.0 [M−H]⁻; Calculated: 201.0

### Step 6: 4-((2-Cyclopropyl-6-isopropyl-7-oxo-[1,2,4]triazolo[1,5-a]pyrimidin-4(7H)-yl)methyl)-2-methylbenzenesulfonamide

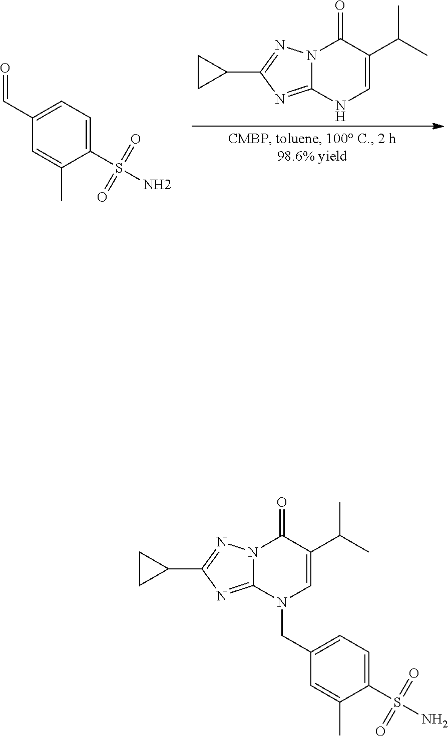 [SMILES: Cc1cc(C=O)ccc1S(N)(=O)=O ; CC(C)c1c[nH]c2nc(C3CC3)nn2c1=O ; Cc1cc(Cn2cc(C(C)C)c(=O)n3nc(C4CC4)nc23)ccc1S(N)(=O)=O]

To a stirred solution of 4-(hydroxymethyl)-2-methylbenzenesulfonamide (300 mg, 1.50 mmol, 1 equiv) and 2-cyclopropyl-6-isopropyl-4H-[1,2,4]triazolo[1,5-a]pyrimidin-7-one (325.37 mg, 1.50 mmol, 1 equiv) in Toluene (10 mL) were added CMBP (3.6 g, 14.9 mmol, 10 equiv) at 25° C. under nitrogen r atmosphere. The resulting mixture was stirred for an additional 2 h at 100° C. The desired product could be detected by LCMS. The mixture was allowed to cool down to 25° C. The resulting mixture was diluted with water (20 mL). The resulting mixture was extracted with EtOAc (3×20 mL). The combined organic layers were washed with brine (50 mL), and dried over anhydrous Na₂SO₄. After filtration, the filtrate was concentrated under reduced pressure. The residue was purified by silica gel column chromatography, eluted with PE/EA (1:1) to afford 4-({2-cyclopropyl-6-isopropyl-7-oxo-[1,2,4]triazolo[1,5-a]pyrimidin-4-yl}methyl)-2-methylbenzenesulfonamide. The crude product (300 mg) was purified by Prep-HPLC with the following conditions (Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 15% B to 30% B in 8 min; Wave Length: 254 nm/220 nm nm; RT1 (min): 5.58/9.43), the pure fraction was concentrated then lyophilized to afford 4-({2-cyclopropyl-6-isopropyl-7-oxo-[1,2,4]triazolo[1,5-a]pyrimidin-4-yl}methyl)-2-methylbenzenesulfonamide.

ESI-MS m/z=402.15 [M+H]⁺, Calculated: 401.2

¹H NMR (300 MHZ, DMSO-d₆) δ 8.04-7.98 (m, 1H), 7.82 (d, J=8.1 Hz, 1H), 7.38 (d, J=2.6 Hz, 3H), 7.30 (dd, J=8.1, 1.9 Hz, 1H), 5.35 (br, 2H), 2.99 (m, 1H), 2.57 (s, 3H), 2.05 (m, 1H), 1.19 (d, J=6.9 Hz, 6H), 1.07-0.82 (m, 4H).

Total proton count from structure: 23

Total proton count from spectrum: 23

### Compound 23: 4-((2-cyclopropyl-6-isopropyl-7-oxo-[1,2,4]triazolo[1,5-a]pyrimidin-4(7H)-yl)methyl)-2-isopropylbenzenesulfonamide

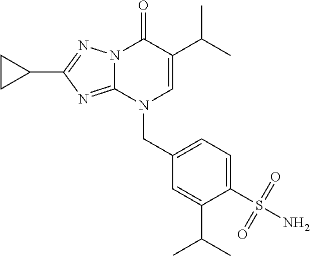 [SMILES: CC(C)c1cc(Cn2cc(C(C)C)c(=O)n3nc(C4CC4)nc23)ccc1S(N)(=O)=O]

### Step 1: 4-Fluoro-3-(prop-1-en-2-yl)benzonitrile

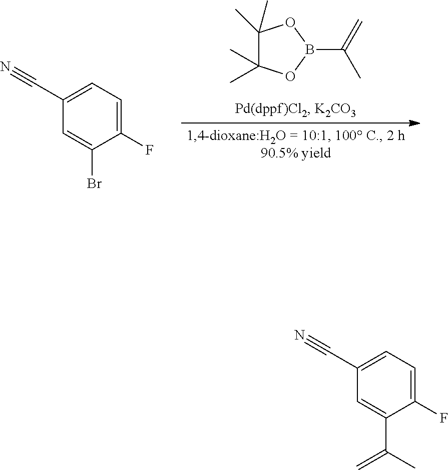

To a stirred solution of 3-bromo-4-fluorobenzonitrile (10.00 g, 49.9 mmol, 1.0 equiv) and 4,4,5,5-tetramethyl-2-(prop-1-en-2-yl)-1,3,2-dioxaborolane (12.60 g, 74.9 mmol, 1.5 equiv) in 1,4-dioxane (100 mL) and H₂O (10 mL) were added Pd (dppf) Cl₂ (3.66 g, 5.00 mmol, 0.1 equiv) and K₂CO₃ (13.82 g, 99.9 mmol, 2.0 equiv) at 20° C. under nitrogen atmosphere. The resulting mixture was stirred for 2 h at 100° C. under nitrogen atmosphere. The mixture was allowed to cool down to 20° C. The resulting mixture was extracted with EtOAc (3×200 mL). The combined organic layers were washed with brine (3×50 mL), and dried over anhydrous Na₂SO₄. After filtration, the filtrate was concentrated under reduced pressure. The residue was purified by silica gel column chromatography, eluted with PE/EA (2:1) to afford 4-fluoro-3-(prop-1-en-2-yl)benzonitrile.

¹H NMR (400 MHZ, DMSO-d₆) δ 7.94 (dd, J=7.3, 2.2 Hz, 1H), 7.91-7.83 (m, 1H), 7.46 (dd, J=11.0, 8.5 Hz, 1H), 5.43-5.36 (m, 1H), 5.34 (q, J=1.2 Hz, 1H), 2.11 (q, J=1.4 Hz, 3H).

### Step 2: 4-(Benzylsulfanyl)-3-(prop-1-en-2-yl)benzonitrile

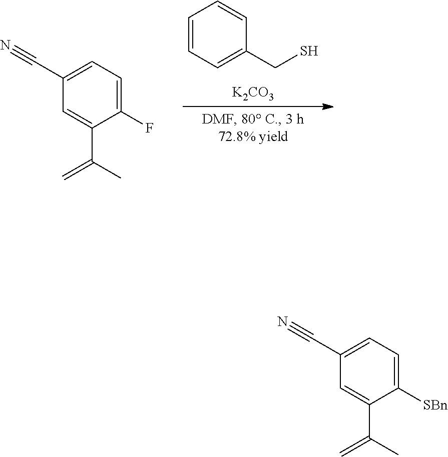

To a stirred solution of 4-fluoro-3-(prop-1-en-2-yl)benzonitrile (5.00 g, 31.0 mmol, 1.0 equiv) and K₂CO₃ (8.57 g, 62.0 mmol, 2.0 equiv) in DMF (20 mL) was added benzyl mercaptan (4.62 g, 37.2 mmol, 1.2 equiv) dropwise at 20° C. under nitrogen atmosphere. The resulting mixture was stirred for 3 h at 80° C. under nitrogen atmosphere. The desired product could be detected by LCMS. The mixture was allowed to cool down to 20° C. The resulting mixture was diluted with EtOAc (200 mL). The resulting mixture was washed with 5×200 ml of water. The organic layers were dried over anhydrous Na₂SO₄. After filtration, the filtrate was concentrated under reduced pressure. The residue was purified by reversed-phase flash chromatography with the following conditions: column: C18; mobile phase: MeCN in Water (0.1% NH₃·H₂O), 50% to 70% gradient in 20 min; detector: UV 254 nm. This resulted in 4-(benzylsulfanyl)-3-(prop-1-en-2-yl)benzonitrile.

ESI-MS m/z=264.1 [M−H]⁻; Calculated: 265.1.

### Step 3: 4-(Benzylsulfanyl)-3-isopropylbenzonitrile

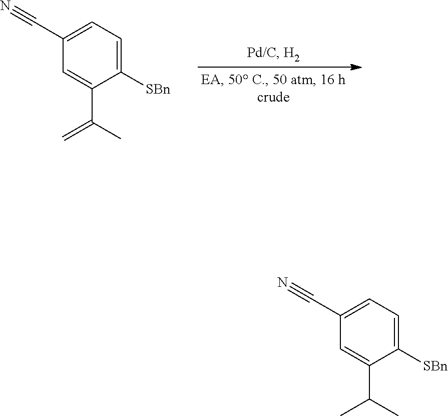

To a solution of 4-(benzylsulfanyl)-3-(prop-1-en-2-yl)benzonitrile (7.00 g, 26.3 mmol, 1.0 equiv) in EtOAc (70 mL) was added Pd/C (10%, 5 g) in a pressure tank. The mixture was hydrogenated at 50° C. under 50 atm of hydrogen pressure for 16 h. The desired product could be detected by LCMS. The solution filtered through a Celite pad and concentrated under reduced pressure. This resulted in 4-(benzylsulfanyl)-3-isopropylbenzonitrile (crude).

ESI-MS m/z=268.1 [M+H]⁺; Calculated: 267.1

### Step 4: 4-Cyano-2-isopropylbenzenesulfonyl chloride

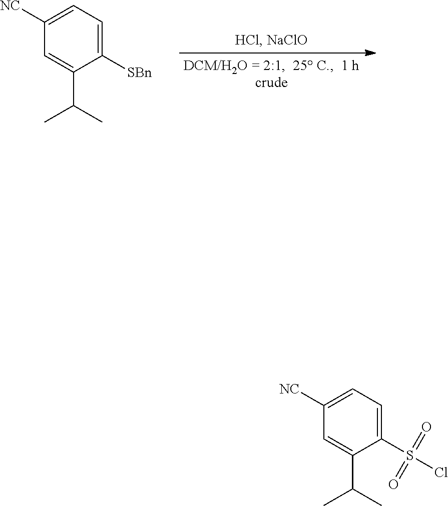

To a stirred solution of 4-(benzylsulfanyl)-3-isopropylbenzonitrile (6.00 g, 22.4 mmol, 1.0 equiv) in DCM/H₂O (100 mL/50 mL) were added conc. HCl (30 mL) and NaClO (116 mL, 172.7 mmol, 7.7 equiv, 10% in H₂O) slowly at 0° C. under nitrogen atmosphere. The resulting mixture was stirred for 1 h at 25° C. under nitrogen atmosphere. The desired product could be detected by LCMS. The reaction mixture was poured into water (80 mL). The resulting mixture was extracted with EtOAc (500 mL). The combined organic layers were washed with brine (2×100 mL), and dried over anhydrous Na₂SO₄. After filtration, the filtrate was concentrated under reduced pressure. This resulted in 4-cyano-2-isopropylbenzenesulfonyl chloride (crude).

ESI-MS m/z=224.0 [M−H]⁻ as sulfonic acid; Calculated: 243.0

### Step 5:4-Cyano-2-isopropylbenzenesulfonamide

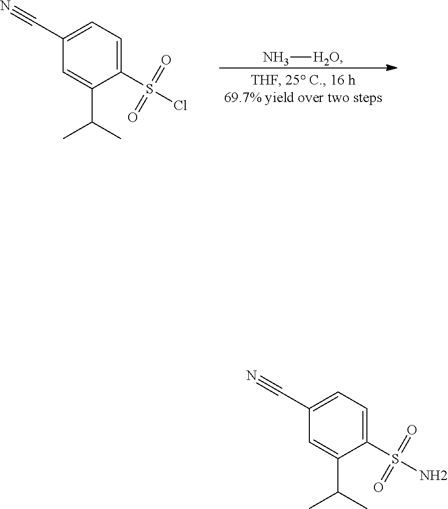

To a stirred solution of 4-cyano-2-isopropylbenzenesulfonyl chloride (5.30 g, 21.7 mmol, 1.0 equiv) in THF (250 mL) was added NH₃·H₂O (50 mL, 326.2 mmol, 15.0 equiv, 25% wt) dropwise at 25° C. The resulting mixture was stirred for 16 h at 25° C. The desired product could be detected by LCMS. The resulting mixture was concentrated under reduced pressure. The residue was purified by reversed-phase flash chromatography with the following conditions: column: C18; mobile phase: MeCN in Water (0.1% FA), 20% to 50% gradient in 20 min; detector: UV 254 nm. This resulted in 4-cyano-2-isopropylbenzenesulfonamide.

ESI-MS m/z=223.0 [M−H]⁻; Calculated: 224.1.

¹H NMR (400 MHz, DMSO-d₆) δ 8.10 (d, J=1.7 Hz, 1H), 7.97 (d, J=8.3 Hz, 1H), 7.84 (dd, J=8.2, 1.7 Hz, 1H), 7.76 (s, 2H), 3.88-3.84 (m, 1H), 1.24 (d, J=6.8 Hz, 6H).

### Step 6:4-Formyl-2-isopropylbenzenesulfonamide

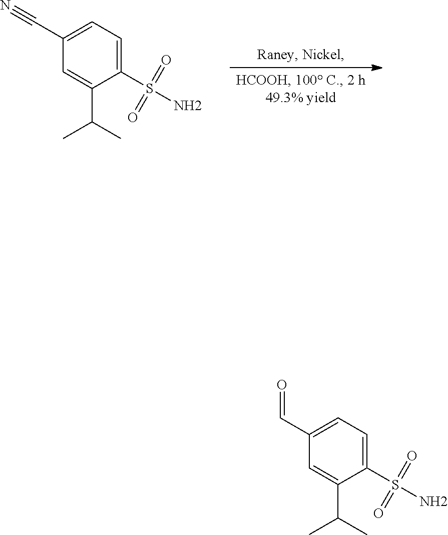

A solution of 4-cyano-2-isopropylbenzenesulfonamide (500 mg, 2.22 mmol, 1.0 equiv) and raney nickel in HCOOH (5 mL) was stirred for 2 h at 100° C. under nitrogen atmosphere. The desired product could be detected by LCMS. The mixture was allowed to cool down to 25° C. The resulting mixture was concentrated under reduced pressure. The residue was purified by reversed-phase flash chromatography with the following conditions: column: C18; mobile phase: MeCN in Water (0.1% FA), 10% to 30% gradient in 20 min; detector: UV 254 nm. This resulted in 4-formyl-2-isopropylbenzenesulfonamide.

ESI-MS m/z=226.0 [M−H]⁻; Calculated: 227.1.

### Step 7: 4-(Hydroxymethyl)-2-isopropylbenzenesulfonamide

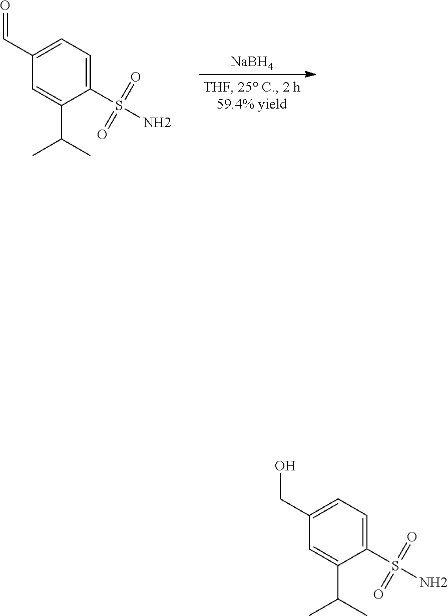

To a stirred solution of 4-formyl-2-isopropylbenzenesulfonamide (200 mg, 0.88 mmol, 1.0 equiv) in THF (2 mL) was added NaBH₄ (49 mg, 1.32 mmol, 1.5 equiv) in portions at 0° C. The resulting mixture was stirred for 2 h at 25° C. The desired product could be detected by LCMS. The reaction was quenched by the addition of Water (5 mL) at 0° C. The resulting mixture was extracted with EtOAc (3×20 mL). The combined organic layers were washed with brine (2×10 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (0.1% FA), 10% to 30% gradient in 10 min; detector: UV 254 nm. This resulted in 4-(hydroxymethyl)-2-isopropylbenzenesulfonamide.

ESI-MS m/z=228.0 [M−H]⁻; Calculated: 229.1.

¹H NMR (400 MHZ, DMSO-d₆) δ 7.78 (d, J=8.1 Hz, 1H), 7.48 (d, J=1.7 Hz, 1H), 7.41 (s, 2H), 7.25 (dd, J=8.3, 1.7 Hz, 1H), 5.33 (t, J=5.8 Hz, 1H), 4.54 (d, J=5.3 Hz, 2H), 3.87-3.83 (m, 1H), 1.21 (d, J=6.8 Hz, 6H).

### Step 8: 4-(Bromomethyl)-2-isopropylbenzenesulfonamide

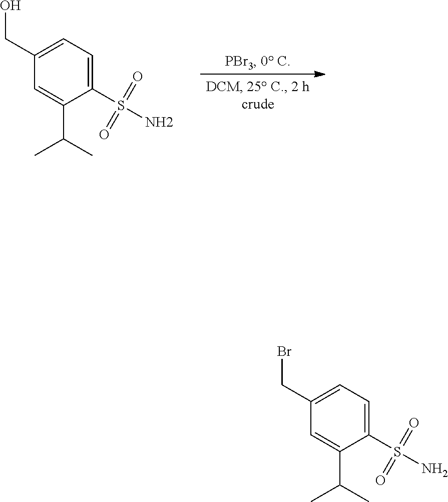

To a stirred solution of 4-(hydroxymethyl)-2-isopropylbenzenesulfonamide (100 mg, 0.43 mmol, 1.0 equiv) in DCM (1 mL) was added PBr3 (354 mg, 1.30 mmol, 3.0 equiv) dropwise at 0° C. under nitrogen atmosphere. The resulting mixture was stirred for 2 h at 25° C. under nitrogen atmosphere. The desired product could be detected by LCMS. The resulting mixture was diluted with EtOAc (10 mL). The resulting mixture was washed with 2×5 ml of water. The combined organic layers were dried over anhydrous Na₂SO₄. After filtration, the filtrate was concentrated under reduced pressure. The crude product was used in the next step directly without further purification.

ESI-MS m/z=289.9 & 291.9 [M−H]⁻; Calculated: 291.0 & 293.0.

### Step 9: 4-((2-Cyclopropyl-6-isopropyl-7-oxo-[1,2,4]triazolo[1,5-a]pyrimidin-4(7H)-yl)methyl)-2-isopropylbenzenesulfonamide

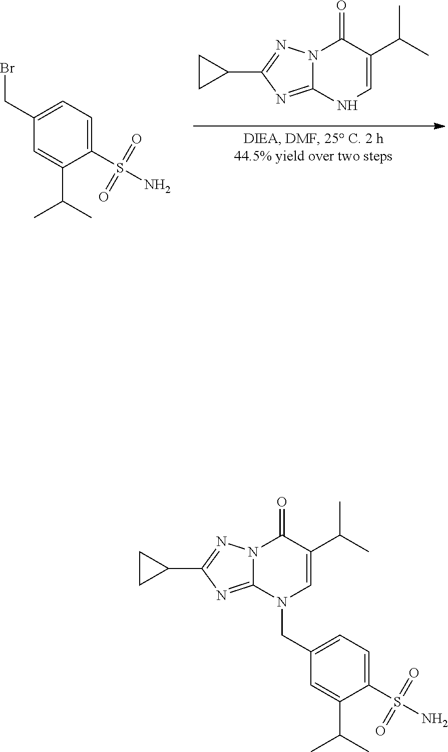 [SMILES: CC(C)c1cc(CBr)ccc1S(N)(=O)=O ; CC(C)c1c[nH]c2nc(C3CC3)nn2c1=O ; CC(C)c1cc(Cn2cc(C(C)C)c(=O)n3nc(C4CC4)nc23)ccc1S(N)(=O)=O]

To a stirred solution of 4-(bromomethyl)-2-isopropylbenzenesulfonamide (90 mg, 0.30 mmol, 1.0 equiv) and 2-cyclopropyl-6-isopropyl-4H-[1,2,4]triazolo[1,5-a]pyrimidin-7-one (60 mg, 0.27 mmol, 0.9 equiv) in DMF (1.5 mL) was added DIEA (119 mg, 0.92 mmol, 3.0 equiv) dropwise at 25° C. under nitrogen atmosphere. The resulting mixture was stirred for 2 h at 25° C. under nitrogen atmosphere. The desired product could be detected by LCMS. The resulting mixture was diluted with EtOAc (20 mL). The resulting mixture was washed with 5×15 ml of water. The organic layer was dried over anhydrous Na₂SO₄. After filtration, the filtrate was concentrated under reduced pressure. The crude product was purified by Prep-HPLC with the following conditions (Column: Xselect CSH C18 OBD Column 30*150 mm, 5 μm; Mobile Phase A: Water (0.1% FA), Mobile Phase B: ACN; Flow rate: 60 mL/min mL/min; Gradient: 30% B to 46% B in 8 min; Wave Length: 254 nm/220 nm nm; RT1 (min): 9.67), the pure fraction was concentrated then lyophilized to afford to afford 4-({2-cyclopropyl-6-isopropyl-7-oxo-[1,2,4]triazolo[1,5-a]pyrimidin-4-yl}methyl)-2-isopropylbenzenesulfonamide.

ESI-MS m/z=430.15 [M+H]⁺; Calculated: 429.2.

¹H NMR (400 MHZ, DMSO-d₆) δ 8.07 (s, 1H), 7.78 (d, J=8.2 Hz, 1H), 7.65 (d, J=1.8 Hz, 1H), 7.47 (s, 2H), 7.27 (dd, J=8.2, 1.8 Hz, 1H), 5.34 (s, 2H), 3.83 (p, J=6.7 Hz, 1H), 3.02-2.98 (m, 1H), 2.05 (tt, J=8.3, 4.8 Hz, 1H), 1.19 (dd, J=6.9, 5.3 Hz, 12H), 1.04-0.87 (m, 4H).

Total proton count from structure: 27.

Total proton count from spectrum: 27.

### Compound 24: 4-((2-cyclopropyl-6-isopropyl-7-oxo-[1,2,4]triazolo[1,5-a]pyrimidin-4(7H)-yl)methyl)-3-hydroxybenzenesulfonamide

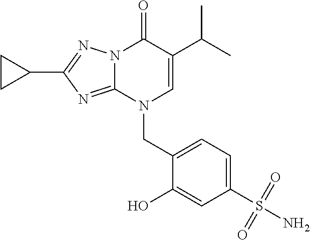 [SMILES: CC(C)c1cn(Cc2ccc(S(N)(=O)=O)cc2O)c2nc(C3CC3)nn2c1=O]

### Step 1: 4-(Benzylsulfanyl)-2-methoxybenzaldehyde

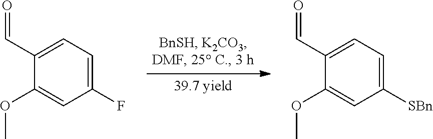

To a solution of 4-fluoro-2-methoxybenzaldehyde (3.00 g, 19.5 mmol, 1.0 equiv) and benzyl mercaptan (2.66 g, 21.4 mmol, 1.1 equiv) in DMF (50 mL) was added K₂CO₃ (5.38 g, 38.9 mmol, 2.0 equiv) at 25° C. under nitrogen atmosphere. The resulting mixture was stirred for 3 h at 25° C. under nitrogen atmosphere. The reaction was monitored by LCMS. The resulting mixture was diluted with EtOAc (250 mL). The combined organic layers were washed with water (5×200 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (0.1% FA), 20% to 65% gradient in 40 min; detector: UV 254 nm. to afford 4-(benzylsulfanyl)-2-methoxybenzaldehyde.

ESI-MS m/z=259.0 [M+H]⁺; Calculated: 258.1.

### Step 2: 4-(Benzylsulfanyl)-2-bromobenzonitrile

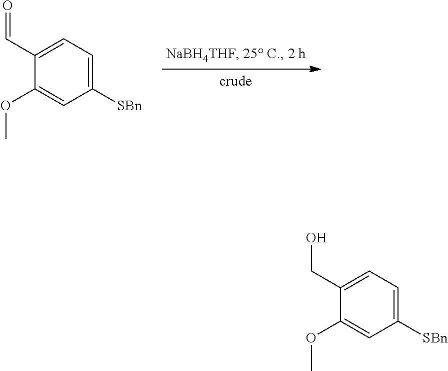

To a solution of 4-(benzylsulfanyl)-2-methoxybenzaldehyde (1.80 g, 6.97 mmol, 1.0 equiv) in THF (20 mL) was added NaBH₄ (1.32 g, 34.8 mmol, 5.0 equiv) at 0° C. under nitrogen atmosphere. The resulting mixture was stirred for 2 h at 25° C. under nitrogen atmosphere. The reaction was monitored by LCMS. The reaction was quenched with Water at 0° C. The resulting mixture was extracted with EtOAc (3×50 mL), and dried over anhydrous Na₂SO₄. After filtration, the filtrate was concentrated under reduced pressure to afford [4-(benzylsulfanyl)-2-methoxyphenyl]methanol (crude).

ESI-MS m/z=259.0 [M−H]⁻; Calculated: 260.1.

### Step 3: 4{[4-(Benzylsulfanyl)-2-methoxyphenyl]methyl}-2-cyclopropyl-6-isopropyl-[1,2,4]triazolo[1,5-a]pyrimidin-7-one

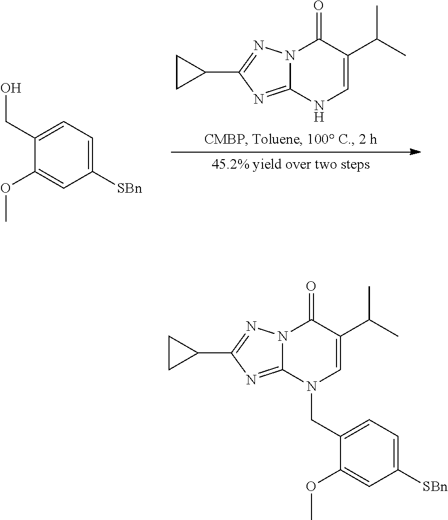

To a solution of [4-(benzylsulfanyl)-2-methoxyphenyl]methanol (1.00 g, 3.84 mmol, 1.0 equiv) and 2-cyclopropyl-6-isopropyl-4H-[1,2,4]triazolo[1,5-a]pyrimidin-7-one (1.01 g, 4.61 mmol, 1.2 equiv) in toluene (15 mL) was added CMBP (13.9 g, 57.7 mmol, 15.0 equiv) at 25° C. under nitrogen atmosphere. The resulting mixture was stirred for 2 h at 100° C. under nitrogen atmosphere. The mixture was allowed to cool down to 25° C. The resulting mixture was diluted with water (50 mL). The resulting mixture was extracted with EtOAc (3×50 mL), The combined organic layers were washed with brine (1×50 mL), and dried over anhydrous Na₂SO₄. After filtration, the filtrate was concentrated under reduced pressure. The residue was purified by reversed-phase flash chromatography with the following conditions: column; C18; mobile phase: MeCN in Water (0.1% FA), 110% to 60% gradient in 40 min; detector: UV 254 nm. to afford 4{[4-(benzylsulfanyl)-2-methoxyphenyl]methyl}-2-cyclopropyl-6-isopropyl-[1,2,4]triazolo[1,5-a]pyrimidin-7-one.

ESI-MS m/z=461.1 [M+H]⁺; Calculated: 460.2.

### Step 4: 4-({2-Cyclopropyl-6-isopropyl-7-oxo-[1,2,4]triazolo[1,5-a]pyrimidin-4-yl}methyl)-3-Step methoxybenzenesulfonyl chloride

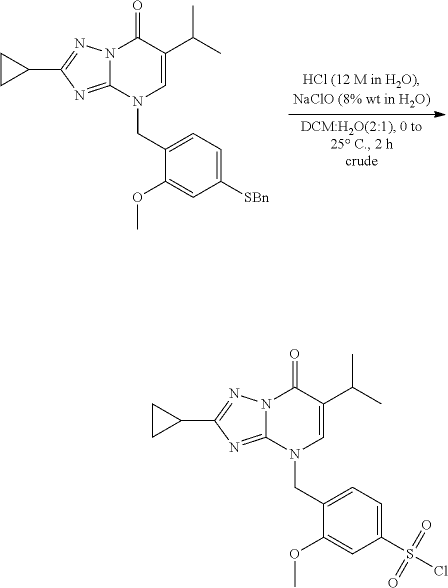

To a solution of 4{[4-(benzylsulfanyl)-2-methoxyphenyl]methyl}-2-cyclopropyl-6-isopropyl-[1,2,4]triazolo[1,5-a]pyrimidin-7-one (600 mg, 1.30 mmol, 1.0 equiv) in DCM (24 mL)/H₂O (12 mL) was added HCl (1.09 mL, 13.0 mmol, 10.0 equiv, 12 M in H₂O) and NaClO (3.31 mL, 3.91 mmol, 3.0 equiv, 8% wt in H₂O) dropwise at 0° C. under nitrogen atmosphere. The resulting mixture was stirred for 2 h at 25° C. under nitrogen atmosphere. The reaction was monitored by LCMS. The resulting mixture was extracted with CH₂Cl₂ (2×20 mL), and dried over anhydrous Na₂SO₄. After filtration, the filtrate was concentrated under reduced pressure. The crude product mixture was used in the next step directly without further purification.

ESI-MS m/z=437.1 [M+H]⁺; Calculated: 436.1.

### Step 5: 4-({2-Cyclopropyl-6-isopropyl-7-oxo-[1,2,4]triazolo[1,5-a]pyrimidin-4-yl}methyl)-3-methoxybenzenesulfonamide

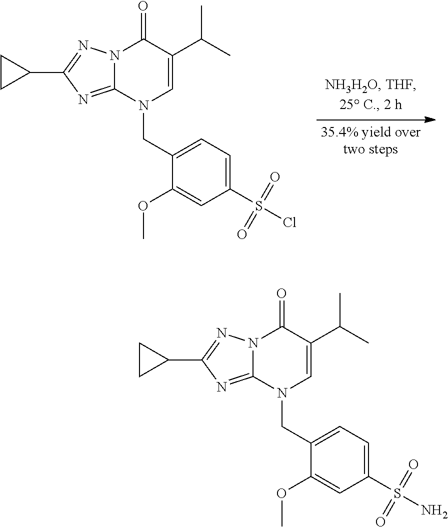 [SMILES: COc1cc(S(=O)(=O)Cl)ccc1Cn1cc(C(C)C)c(=O)n2nc(C3CC3)nc12 ; COc1cc(S(N)(=O)=O)ccc1Cn1cc(C(C)C)c(=O)n2nc(C3CC3)nc12]

To a solution of 4-({2-cyclopropyl-6-isopropyl-7-oxo-[1,2,4]triazolo[1,5-a]pyrimidin-4-yl}methyl)-3-methoxybenzenesulfonyl chloride (650 mg, 1.49 mmol, 1.0 equiv) in THF (10 mL) was added NH₃·H₂O (2.17 mL, 55.7 mmol, 37.4 equiv, 30% wt in H₂O) at 25° C. under nitrogen atmosphere. The resulting mixture was stirred for 2 h at 25° C. under nitrogen atmosphere. The reaction was monitored by LCMS. The resulting mixture was concentrated under reduced pressure. The residue was purified by reversed-phase flash chromatography with the following conditions: column: C18; mobile phase: MeCN in Water (0.1% FA), 10% to 50% gradient in 30 min; detector: UV 254 nm. to afford 4-({2-cyclopropyl-6-isopropyl-7-oxo-[1,2,4]triazolo[1,5-a]pyrimidin-4-yl}methyl)-3-methoxybenzenesulfonamide.

ESI-MS m/z=416.1 [M−H]; Calculated: 417.1.

¹H NMR (300 MHZ, DMSO-d₆) δ 7.92 (s, 1H), 7.48-7.42 (m, 1H), 7.37 (d, J=8.4 Hz, 3H), 7.29 (d, J=7.8 Hz, 1H), 5.30 (s, 2H), 3.91 (d, J=2.1 Hz, 3H), 3.00 (p, J=6.9 Hz, 1H), 2.01 (dq, J=8.2, 4.8, 4.1 Hz, 1H), 1.19 (d, J=6.9 Hz, 6H), 1.02-0.94 (m, 2H), 0.86 (dt, J=6.8, 3.5 Hz, 2H).

### Step 6: 4-((2-Cyclopropyl-6-isopropyl-7-oxo-[1,2,4]triazolo[1,5-a]pyrimidin-4(7H)-yl)methyl)-3-hydroxybenzenesulfonamide

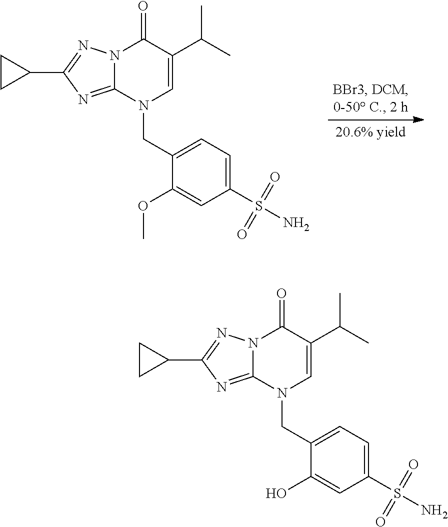 [SMILES: COc1cc(S(N)(=O)=O)ccc1Cn1cc(C(C)C)c(=O)n2nc(C3CC3)nc12 ; CC(C)c1cn(Cc2ccc(S(N)(=O)=O)cc2O)c2nc(C3CC3)nn2c1=O]

To a solution of 4-({2-cyclopropyl-6-isopropyl-7-oxo-[1,2,4]triazolo[1,5-a]pyrimidin-4-yl}methyl)-3-methoxybenzenesulfonamide (200 mg, 0.48 mmol, 1.0 equiv) in DCM (3 mL) was added BBr₃ (1.20 g, 4.79 mmol, 10.0 equiv) at 0° C. under nitrogen atmosphere. The resulting mixture was stirred for 2 h at 50° C. under nitrogen atmosphere. The reaction was monitored by LCMS. The mixture was allowed to cool down to 25° C. The reaction was quenched with Water/Ice at 0° C. The resulting mixture was extracted with CH₂Cl₂ (3×20 mL), and dried over anhydrous Na₂SO₄. After filtration, the filtrate was concentrated under reduced pressure. The crude product was purified by Prep-HPLC with the following conditions (Column: XSelect CSH Prep C18 OBD Column, 30*150 mm, 5 m; Mobile Phase A: Water (0.1% FA), Mobile Phase B: ACN; Flow rate: 60 mL/min mL/min; Gradient: 22% B to 42% B in 10 min; Wave Length: 254 nm/220 nm nm; RT1 (min): 8.43) the pure fraction was concentrated then lyophilized to afford 4-((2-cyclopropyl-6-isopropyl-7-oxo-[1,2,4]triazolo[1,5-a]pyrimidin-4(7H)-yl)methyl)-3-hydroxybenzenesulfonamide.

ESI-MS m/z=404.0 [M+H]⁺; Calculated: 403.1.

¹H NMR (300 MHZ, DMSO-d₆) δ 10.67 (s, 1H), 7.91 (s, 1H), 7.36-7.10 (m, 5H), 5.26 (s, 2H), 2.98 (p, J=6.9 Hz, 1H), 2.08-2.00 (m, 1H), 1.17 (d, J=6.9 Hz, 6H), 1.05-0.94 (m, 2H), 0.88 (q, J=3.6 Hz, 2H).

Total proton count from structure: 21.

Total proton count from spectrum: 21.

### Compound 25: 2-cyclopropoxy-6-isopropyl-4-(4-(S-methylsulfonimidoyl)benzyl)-[1,2,4]triazolo[1,5-a]pyrimidin-7(4H)-one

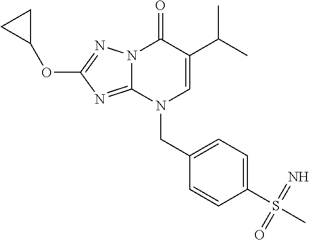 [SMILES: CC(C)c1cn(Cc2ccc(S(C)(=N)=O)cc2)c2nc(OC3CC3)nn2c1=O]

### Step 1: 6-Isopropyl-2-(methylthio)-[1,2,4]triazolo[1,5-a]pyrimidin-7(4H)-one

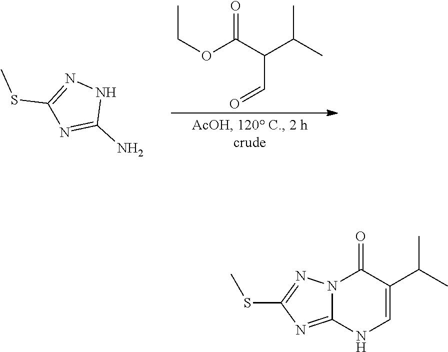

A solution of 5-(methylsulfanyl)-2H-1,2,4-triazol-3-amine (5.00 g, 38.4 mmol, 1.0 equiv) and ethyl 2-formyl-3-methylbutanoate (6.68 g, 42.3 mmol, 1.1 equiv) in AcOH (50 mL) was stirred for 2 h at 120° C. under nitrogen atmosphere. The desired product could be detected by LCMS. The mixture was allowed to cool down to 25° C. The precipitated solids were collected by filtration and washed with water (3×10 mL) to afford 6-isopropyl-2-(methylthio)-[1,2,4]triazolo[1,5-a]pyrimidin-7(4H)-one (crude).

ESI-MS m/z=225.00 [M+H]⁺; Calculated: 224.1.

### Step 2: 6-Isopropyl-2-(methylsulfonyl)-[1,2,4]triazolo[1,5-a]pyrimidin-7(4H)-one

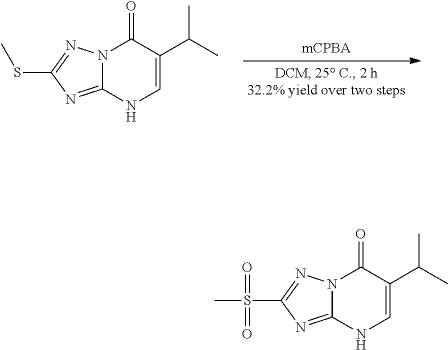 [SMILES: CSc1nc2[nH]cc(C(C)C)c(=O)n2n1 ; CC(C)c1c[nH]c2nc(S(C)(=O)=O)nn2c1=O]

To a stirred solution of 6-isopropyl-2-(methylthio)-[1,2,4]triazolo[1,5-a]pyrimidin-7(4H)-one (2.00 g, 8.92 mmol, 1.0 equiv) in DCM (30 mL) was added mCPBA (7.69 g, 44.6 mmol, 5.0 equiv) in portions at 0° C. The resulting mixture was stirred for 2 h at 25° C. under nitrogen atmosphere. The desired product could be detected by LCMS. The precipitated solids were collected by filtration and washed with DCM (3×5 mL). The residue was dissolved in MeCN (75 mL) and H₂O (75 mL). The reaction was quenched by the addition of 10% Na₂S₂O₃ (aq.) (50 mL) at 25° C. The resulting mixture was concentrated under reduced pressure. The residue was purified by reversed-phase flash chromatography with the following conditions (column: C18; mobile phase: MeCN in Water (0.1% FA), 0% to 50% gradient in 10 min; detector: UV 254 nm), to afford 6-isopropyl-2-(methylsulfonyl)-[1,2,4]triazolo[1,5-a]pyrimidin-7(4H)-one.

ESI-MS m/z=257.10 [M+H]⁺; Calculated: 256.1.

### Step 3: 6-Isopropyl-4-(4-(S-methylsulfonimidoyl)benzyl)-2-(methylsulfonyl)-[1,2,4]triazolo[1,5-a]pyrimidin-7(4H)-one

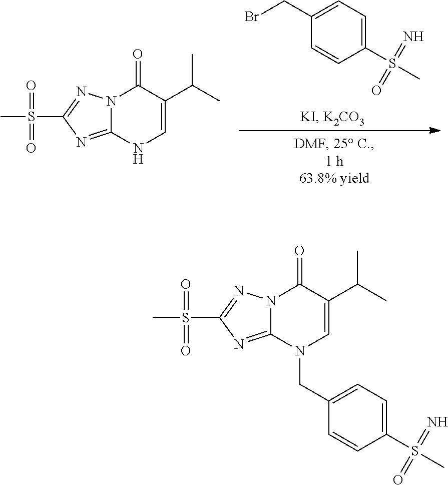

To a stirred solution of 6-isopropyl-2-(methylsulfonyl)-[1,2,4]triazolo[1,5-a]pyrimidin-7(4H)-one (600 mg, 2.34 mmol, 1.0 equiv) and [4-(bromomethyl)phenyl](imino)methyl-lambda6-sulfanone (871 mg, 3.51 mmol, 1.5 equiv) in DMF (10 mL) were added KI (389 mg, 2.34 mmol, 1.0 equiv) and K₂CO₃ (647 mg, 4.68 mmol, 2.0 equiv) at 25° C. The resulting mixture was stirred for 1 h at 25° C. under nitrogen atmosphere. The desired product could be detected by LCMS. The resulting mixture was filtered, the filter cake was washed with DMF (2×3 mL). The residue was purified by reversed-phase flash chromatography with the following conditions: column: C18; mobile phase: MeCN in Water (0.1% FA), 0% to 40% gradient in 30 min; detector: UV 254 nm to afford 6-isopropyl-4-(4-(S-methylsulfonimidoyl)benzyl)-2-(methylsulfonyl)-[1,2,4]triazolo[1,5-a]pyrimidin-7(4H)-one.

ESI-MS m/z=424.00 [M+H]⁺; Calculated: 423.1.

¹H NMR (400 MHZ, DMSO-d₆) δ 8.22 (s, 1H), 7.94-7.87 (m, 2H), 7.64-7.57 (m, 2H), 5.53 (s, 2H), 4.22 (s, 1H), 3.43 (s, 3H), 3.08-3.00 (m, 4H), 1.25-1.18 (m, 6H).

### Step 4: 2-Cyclopropoxy-6-isopropyl-4-(4-(S-methylsulfonimidoyl)benzyl)-[1,2,4]triazolo[1,5-a]pyrimidin-7(4H)-one

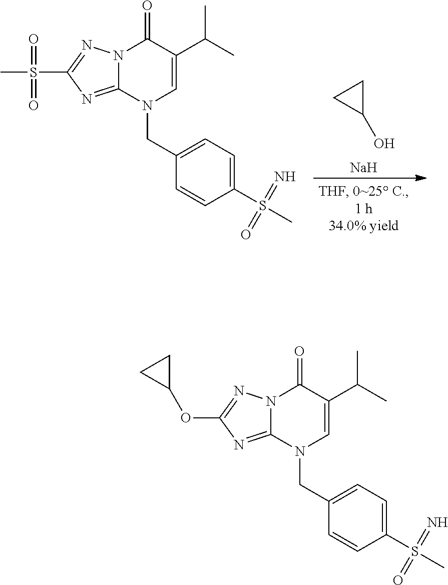

To a stirred solution of cyclopropanol (165 mg, 2.83 mmol, 3.0 equiv) in THF (8 mL) was added NaH (102 mg, 4.25 mmol, 4.5 equiv) in portions at 0° C. The resulting mixture was stirred for 30 min at 0° C. under nitrogen atmosphere. To the above mixture was added 6-isopropyl-4-(4-(S-methylsulfonimidoyl)benzyl)-2-(methylsulfonyl)-[1,2,4]triazolo[1,5-a]pyrimidin-7(4H)-one (400 mg, 0.94 mmol, 1.0 equiv) over 1 min at 0° C. The resulting mixture was stirred for an additional 1 h at 25° C. The desired product could be detected by LCMS. The reaction was quenched by the addition of Water/Ice (200 mL) at 0° C. The resulting mixture was extracted with EtOAc (3×200 mL). The combined organic layers were washed with brine (500 mL), and dried over anhydrous Na₂SO₄. After filtration, the filtrate was concentrated under reduced pressure. The crude product (300 mg) was purified by Prep-HPLC with the following conditions (Column: XBridge Prep OBD C18 Column 30*150 mm, 5 m; Mobile Phase

A: Water (10 mmol/L NH₄HCO₃), Mobile Phase B: ACN; Flow rate: 60 mL/min; Gradient: 15% B to 38% B in 7 min; Wave Length: 254 nm/220 nm nm; RT1 (min): 6), the pure fraction was concentrated then lyophilized to afford 2-cyclopropoxy-6-isopropyl-4-(4-(S-methylsulfonimidoyl)benzyl)-[1,2,4]triazolo[1,5-a]pyrimidin-7(4H)-one.

ESI-MS m/z=402.15 [M+H]⁺; Calculated: 401.2.

¹H NMR (400 MHZ, DMSO-d₆) δ 8.03 (s, 1H), 7.93-7.88 (m, 2H), 7.61-7.54 (m, 2H), 5.38 (s, 2H), 4.29-4.23 (m, 1H), 4.23-4.19 (m, 1H), 3.06-3.02 (m, 3H), 3.02-2.93 (m, 1H), 1.24-1.15 (m, 6H), 0.81-0.71 (m, 4H).

Total proton count from structure: 23.

Total proton count from spectrum: 23.

### Compound 26: 2-cyclopropyl-4-(2,6-difluoro-4-(S-methylsulfonimidoyl)benzyl)-6-isopropyl-[1,2,4]triazolo[1,5-a]pyrimidin-7(4H)-one

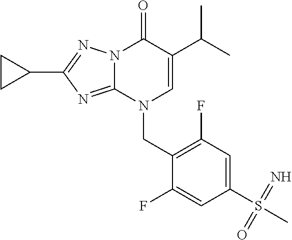 [SMILES: CC(C)c1cn(Cc2c(F)cc(S(C)(=N)=O)cc2F)c2nc(C3CC3)nn2c1=O]

### Step 1: (2,6-Difluoro-4-(methylthio)phenyl)methanol

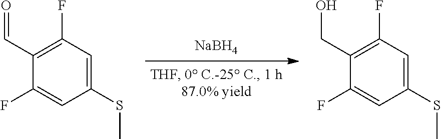

To a stirred solution of 2,6-difluoro-4-(methylsulfanyl)benzaldehyde (2.50 g, 13.3 mmol, 1.0 equiv) in THF (50 mL) was added NaBH₄ (754 mg, 19.9 mmol, 1.5 equiv) in portions at 0° C. The resulting mixture was stirred for 1 h at 25° C. under nitrogen atmosphere. The desired product could be detected by LCMS. The reaction was quenched with sat. NH₄Cl (aq.) (200 mL) at 0° C. The resulting mixture was extracted with EtOAc (3×200 mL), dried over anhydrous Na₂SO₄. After filtration, the filtrate was concentrated under reduced pressure. The residue was purified by silica gel column chromatography, eluted with PE/EA (0% to 20% gradient in 20 min) to afford (2,6-difluoro-4-(methylthio)phenyl)methanol.

ESI-MS m/z=173.10 [M+H-18]⁺; Calculated: 190.0.

¹H NMR (400 MHZ, DMSO-d₆) δ 7.04-6.95 (m, 2H), 5.18 (t, J=5.6 Hz, 1H), 4.49-4.36 (m, 2H), 2.50 (s, 3H).

¹⁹F NMR (377 MHz, DMSO-d₆) δ −115.83.

### Step 2: (3,5-Difluoro-4-(hydroxymethyl)phenyl)(imino)(methyl)-λ⁶-sulfanone

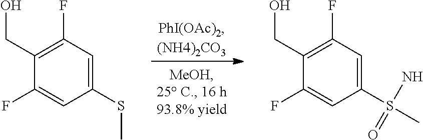

To a stirred solution of (2,6-difluoro-4-(methylthio)phenyl)methanol (1.10 g, 5.78 mmol, 1.0 equiv) and (NH₄)₂CO₃ (1.94 g, 20.2 mmol, 3.5 equiv) in MeOH (22 mL) was added phenyliodine (III) diacetate (8.01 g, 24.9 mmol, 4.3 equiv) in portions at 25° C. The resulting mixture was stirred for 1 h at 25° C. under nitrogen atmosphere. The desired product could be detected by LCMS. The resulting mixture was concentrated under reduced pressure. The residue was purified by silica gel column chromatography, eluted with PE/EA (50% to 90% gradient in 20 min) to afford (3,5-difluoro-4-(hydroxymethyl)phenyl)(imino)(methyl)-l6-sulfanone.

ESI-MS m/z=221.85 [M+H]⁺; Calculated: 221.0.

¹H NMR (400 MHZ, DMSO-d₆) δ 7.61 (d, J=6.4 Hz, 2H), 5.43 (t, J=5.8 Hz, 1H), 4.57-4.53 (m, 2H), 3.14 (d, J=1.2 Hz, 3H). (There's an exchangeable hydrogen that did not appear) ¹⁹F NMR (376 MHZ, DMSO-d₆) δ −112.73.

### Step 3: (4-(Bromomethyl)-3,5-difluorophenyl)(imino)(methyl)-l6-sulfanone

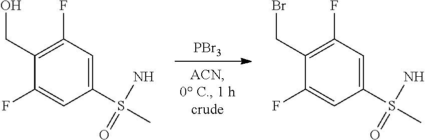

To a stirred solution of (3,5-difluoro-4-(hydroxymethyl)phenyl)(imino)(methyl)-l6-sulfanone (300 mg, 1.36 mmol, 1.0 equiv) in ACN (5 mL) was added PBr₃ (257 mg, 0.95 mmol, 0.7 equiv) dropwise at 0° C. under nitrogen atmosphere. The resulting mixture was stirred for 1 h at 0° C. under nitrogen atmosphere. The desired product could be detected by LCMS. The resulting mixture was concentrated under reduced pressure. This resulted in (4-(bromomethyl)-3,5-difluorophenyl)(imino)(methyl)-l6-sulfanone (crude).

ESI-MS m/z=284.00/286.00 [M+H]⁺; Calculated: 282.9, 284.95.

### Step 4: 2-Cyclopropyl-4-(2,6-difluoro-4-(S-methylsulfonimidoyl)benzyl)-6-isopropyl-[1,2,4]triazolo[1,5-a]pyrimidin-7(4H)-one

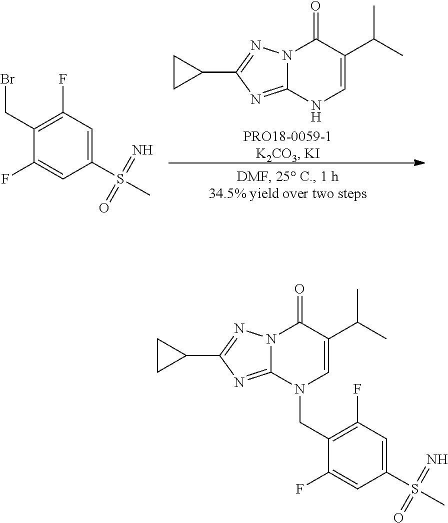 [SMILES: CS(=N)(=O)c1cc(F)c(CBr)c(F)c1 ; CC(C)c1c[nH]c2nc(C3CC3)nn2c1=O ; CC(C)c1cn(Cc2c(F)cc(S(C)(=N)=O)cc2F)c2nc(C3CC3)nn2c1=O]

To a stirred mixture of 2-cyclopropyl-6-isopropyl-4H-[1,2,4]triazolo[1,5-a]pyrimidin-7-one (230 mg, 1.06 mmol, 1.0 equiv) and K₂CO₃ (730 mg, 5.28 mmol, 5.0 equiv) in DMF (6 mL) were added KI (175 mg, 1.06 mmol, 1.0 equiv) and (4-(bromomethyl)-3,5-difluorophenyl)(imino)(methyl)-l6-sulfanone (300 mg, 1.06 mmol, 1.0 equiv) in portions at 25° C. The resulting mixture was stirred for 30 min at 25° C. under nitrogen atmosphere. The desired product could be detected by LCMS. The resulting mixture was diluted with water (50 mL). The resulting mixture was extracted with EtOAc (3×50 mL). The combined organic layers were washed with brine (1×150 mL), and dried over anhydrous Na₂SO₄. After filtration, the filtrate was concentrated under reduced pressure. The crude product (200 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min; Gradient: 15% B to 40% B in 7 min; Wave Length: 254 nm/220 nm; RT1 (min): 6.72), the pure fraction was concentrated then lyophilized to afford 2-cyclopropyl-4-(2,6-difluoro-4-(S-methylsulfonimidoyl)benzyl)-6-isopropyl-[1,2,4]triazolo[1,5-a]pyrimidin-7(4H)-one.

ESI-MS m/z=422.05 [M+H]⁺; Calculated: 421.0.

¹H NMR (400 MHZ, DMSO-d₆) δ 8.04 (s, 1H), 7.65 (d, J=7.1 Hz, 2H), 5.48 (s, 2H), 4.55 (s, 1H), 3.14 (s, 3H), 3.07-2.91 (m, 1H), 2.05-1.94 (m, 1H), 1.20 (d, J=6.9 Hz, 6H), 1.02-0.93 (m, 2H), 0.89-0.77 (m, 2H).

¹⁹F NMR (376 MHZ, DMSO-d₆) δ 109.59.

Total proton count from structure: 21.

Total proton count from spectrum: 21.

### Compound 27: 6-((2-cyclopropyl-6-isopropyl-7-oxo-[1,2,4]triazolo[1,5-a]pyrimidin-4(7H)-yl)methyl)-2-phenylpyridine-3-sulfonamide

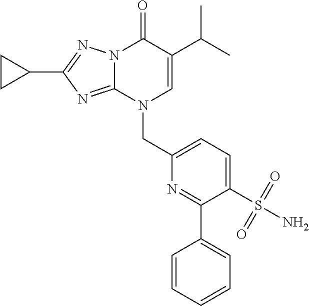 [SMILES: CC(C)c1cn(Cc2ccc(S(N)(=O)=O)c(-c3ccccc3)n2)c2nc(C3CC3)nn2c1=O]

### Step 1: 5-(Benzylthio)-6-bromopicolinic acid

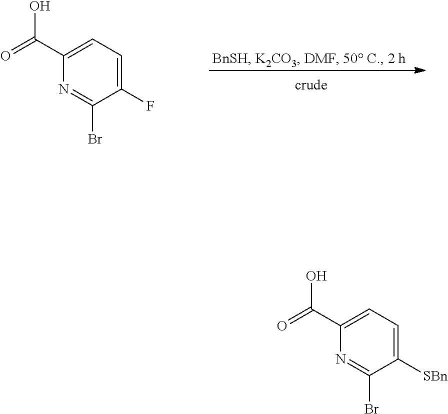

A solution of 6-bromo-5-fluoropyridine-2-carboxylic acid (18.00 g, 81.7 mmol, 1.0 equiv), phenylmethanethiol (11 . . . 52 g, 81.7 mmol, 1.0 equiv) and K₂CO₃ (33.84 g, 245.2 mmol, 3.0 equiv) in DMF (1800 mL) was stirred for 2 h at 50° C. under nitrogen atmosphere. The reaction was monitored by LCMS. The mixture was allowed to cool down to 25° C., the product was precipitated. The precipitated solids were collected by filtration and washed with water (2×500 mL), to afford 5-(benzylthio)-6-bromopicolinic acid (crude). The crude product was used in the next step directly without further purification.

ESI-MS m/z=324.0 [M+H]⁺. Calculated: 323.0.

### Step 2: Ethyl 5-(benzylthio)-6-bromopicolinate

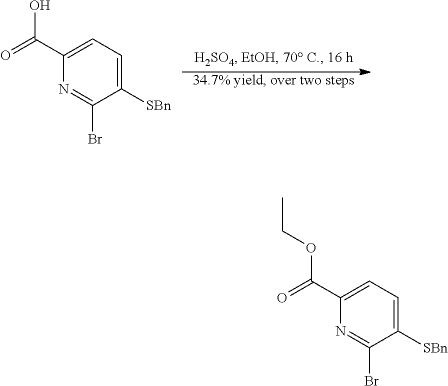

To a stirred solution of 5-(benzylsulfanyl)-6-bromopyridine-2-carboxylic acid (20.00 g, 61.6 mmol, 1.0 equiv) in anhydrous EtOH (200 mL) was added H₂SO₄ (6.00 g, 61.6 mmol, 1.00 equiv) dropwise at room temperature. The resulting mixture was stirred for 16 h at 70° C. under nitrogen atmosphere. The reaction was monitored by LCMS. After completion of reaction, the mixture was allowed to cool down to 25° C. The reaction mixture was quenched by addition of water (200 mL). The aqueous layer was extracted with ethyl acetate (3×200 mL). The combined organic phase was washed with brine (100 mL), and dried over anhydrous sodium sulfate and concentrated under reduced pressure. The residue was purified by silica gel column chromatography, eluted with PE/EA (5:1-3:1) to afford ethyl 5-(benzylthio)-6-bromopicolinate.

ESI-MS m/z=352.0 [M+H]⁺. Calculated: 351.0.

### Step 3: Ethyl 5-(benzylthio)-6-phenylpicolinate

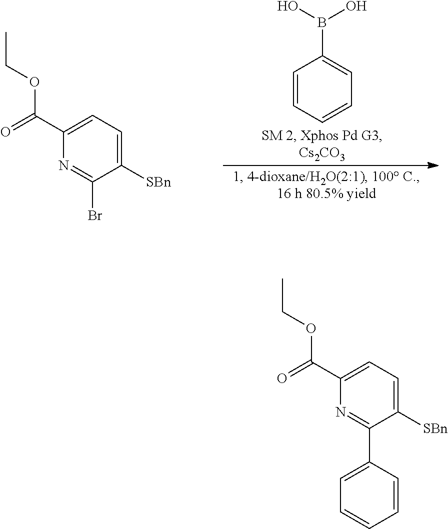

A solution of ethyl 5-(benzylsulfanyl)-6-bromopyridine-2-carboxylate (10.00 g, 28.4 mmol, 1.0 equiv), phenyl boronic acid (6.12 g, 56.7 mmol, 2.0 equiv), Cs₂CO₃ (26.52 g, 85.1 mmol, 3.0 equiv) and 3rd Generation XPhos precatalyst (2.04 g, 2.84 mmol, 0.1 equiv) in 1,4-dioxane (100 mL) and H₂O (10 mL) was stirred for 16 h at 100° C. under nitrogen atmosphere. The reaction was monitored by LCMS. After completion of reaction, the mixture was allowed to cool down to 25° C. The reaction mixture was quenched by addition of water (100 mL). The aqueous layer was extracted with ethyl acetate (3×200 mL). The combined organic phase was washed with brine (200 mL), and dried over anhydrous sodium sulfate and concentrated under reduced pressure to give crude product. The residue was purified by silica gel column chromatography, eluted with PE/EA (5:1-3:1) to afford ethyl 5-(benzylsulfanyl)-6-phenylpyridine-2-carboxylate.

ESI-MS m/z=350.0 [M+H]⁺. Calculated: 349.1.

### Step 4: Ethyl 5-(chlorosulfonyl)-6-phenylpicolinate

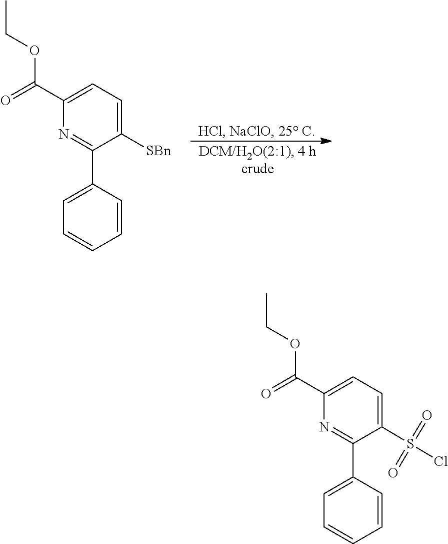

To a stirred solution of 5-(benzylsulfanyl)-6-phenylpyridine-2-carboxylic acid (8.00 g, 24.9 mmol, 1.0 equiv) in DCM:H₂O/2:1 (80 mL) were added conc. HCl (32 mL, 372.8 mmol, 15.0 equiv) and NaClO (160 mL, 191.2 mmol, 7.0 equiv, 8% w.t.) dropwise at 0° C. under nitrogen atmosphere. The resulting mixture was stirred for 4 h at 25° C. under nitrogen atmosphere. The reaction was monitored by LCMS. The resulting mixture was diluted with water (150 mL). The resulting mixture was extracted with CH₂Cl₂ (3×100 mL). The combined organic phase was washed with brine (100 mL), and dried over anhydrous sodium sulfate and concentrated under reduced pressure to afford ethyl 5-(chlorosulfonyl)-6-phenylpicolinate (crude). The crude product was used in the next step directly without further purification.

ESI-MS m/z=306.1 [M−H]⁻ as sulfonic acid; Calculated: 325.0.

### Step 5: Ethyl 6-phenyl-5-sulfamoylpicolinate

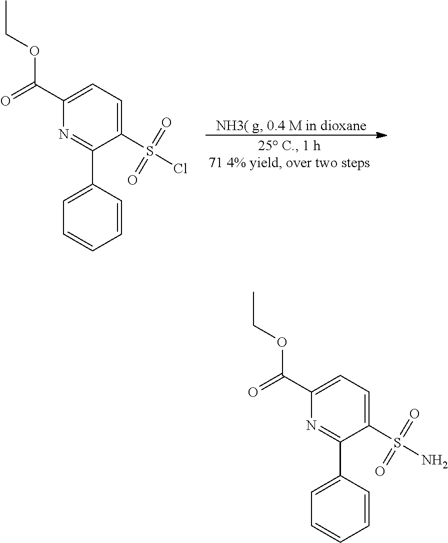 [SMILES: CCOC(=O)c1ccc(S(=O)(=O)Cl)c(-c2ccccc2)n1 ; CCOC(=O)c1ccc(S(N)(=O)=O)c(-c2ccccc2)n1]

A solution of ethyl 5-(chlorosulfonyl)-6-phenylpyridine-2-carboxylate (8.00 g, 24.5 mmol, 1.0 equiv) in NH₃ (g, 0.4M in dioxane, 150 mL) was stirred for 1 h at 25° C. under nitrogen atmosphere. The reaction was monitored by LCMS. The resulting mixture was diluted with water (100 mL). The aqueous layer was extracted with ethyl acetate (2×100 mL). The resulting mixture was concentrated under reduced pressure. The residue was purified by silica gel column chromatography, eluted with PE/EA (3:1-1:1) to afford ethyl 6-phenyl-5-sulfamoylpyridine-2-carboxylate.

ESI-MS m/z=307.1 [M+H]⁺; Calculated: 306.1.

### Step 6: 6-(Hydroxymethyl)-2-phenylpyridine-3-sulfonamide

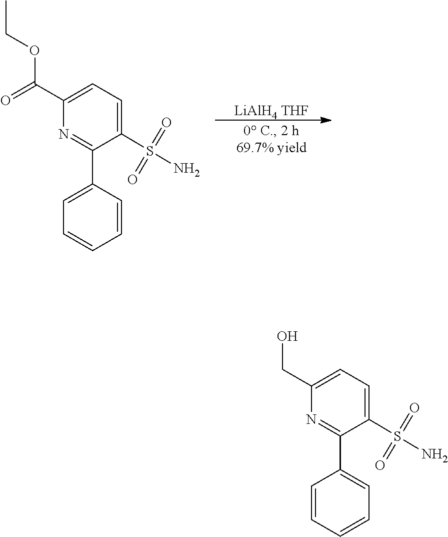 [SMILES: CCOC(=O)c1ccc(S(N)(=O)=O)c(-c2ccccc2)n1 ; NS(=O)(=O)c1ccc(CO)nc1-c1ccccc1 ; "LiAlH4 THF"]

A solution of ethyl 6-phenyl-5-sulfamoylpyridine-2-carboxylate (5.00 g, 16.3 mmol, 1.0 equiv) in THF (50 mL) was treated for 1 minute at 0° C. under nitrogen atmosphere followed by the addition of LiAlH₄ (25 mL, 48.9 mmol, 3.0 equiv, 2 M in THF) dropwise at 0° C. The resulting mixture was stirred for 2 h at 0° C. under nitrogen atmosphere. The reaction was monitored by LCMS. The reaction was quenched by the addition of NH₄Cl (50 mL) at 0° C. The resulting mixture was diluted with water (100 ml). The aqueous layer was extracted with ethyl acetate (3×100 mL). The combined organic phase was washed with brine (100 mL), and dried over anhydrous sodium sulfate and concentrated under reduced pressure to give crude product. The residue was purified by reversed-phase flash chromatography with the following conditions: column: C18 silica gel; mobile phase: ACN in water, 10% to 20% gradient in 15 min; detector: UV 254 nm, to afford 6-(hydroxymethyl)-2-phenylpyridine-3-sulfonamide.

ESI-MS m/z=264.1 [M+H]⁺. Calculated: 265.1.

### Step 7: 6-((2-cyclopropyl-6-isopropyl-7-oxo-[1,2,4]triazolo[1,5-a]pyrimidin-4(7H)-yl)methyl)-2-phenylpyridine-3-sulfonamide

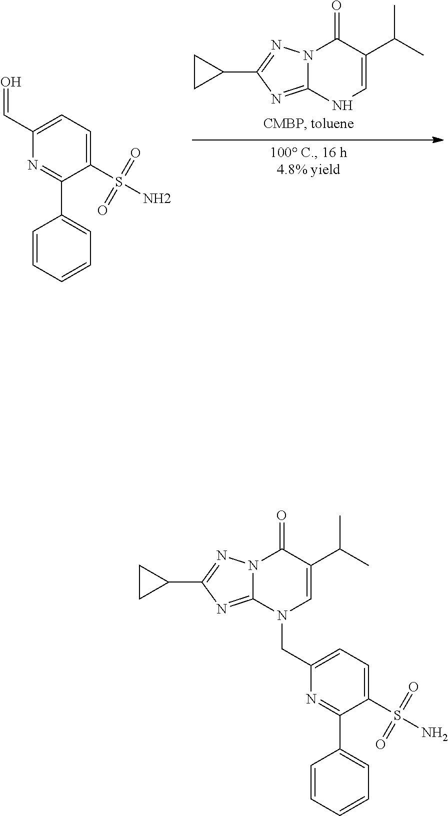 [SMILES: NS(=O)(=O)c1ccc(C=O)nc1-c1ccccc1 ; CC(C)c1c[nH]c2nc(C3CC3)nn2c1=O ; CC(C)c1cn(Cc2ccc(S(N)(=O)=O)c(-c3ccccc3)n2)c2nc(C3CC3)nn2c1=O]

A solution of [5-(benzylsulfanyl)-6-phenylpyridin-2-yl]methanol (3.00 g, 11.4 mmol, 1.4 equiv), 2-cyclopropyl-6-isopropyl-4H-[1,2,4]triazolo[1,5-a]pyrimidin-7-one (1.80 g, 8.25 mmol, 1.0 equiv) and CMBP (9.81 g, 41.3 mmol, 5.0 equiv) in toluene (30 mL) was stirred for 16 h at 100° C. under nitrogen atmosphere. The reaction was monitored by LCMS. The mixture was allowed to cool down to 25° C. The reaction mixture was concentrated under reduced pressure. The residue was purified by silica gel column chromatography, eluted with PE/EA (3:1-1:1) to afford crude product. The crude product was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min mL/min; Gradient: 25% B to 53% B in 7 min; Wave Length: 254 nm/220 nm nm; RT1 (min): 7.02), the pure fraction was concentrated then lyophilized to afford 6-((2-cyclopropyl-6-isopropyl-7-oxo-[1,2,4]triazolo[1,5-a]pyrimidin-4(7H)-yl)methyl)-2-phenylpyridine-3-sulfonamide.

ESI-MS m/z=465.20 [M+H]⁺; Calculated: 464.2.

¹H NMR (400 MHZ, DMSO-d₆) δ 8.40 (d, J=8.3 Hz, 1H), 8.03 (s, 1H), 7.68-7.48 (m, 5H), 7.47-7.33 (m, 3H), 5.57 (s, 2H), 3.08-2.93 (m, 1H), 2.05-1.96 (m, 1H), 1.17 (d, J=6.9 Hz, 6H), 1.03-0.92 (m, 2H), 0.91-0.81 (m, 2H).

Total proton count from structure: 24.

Total proton count from spectrum: 24.

### Compound 28: 4-((6-isopropyl-7-oxo-2-propyl-[1,2,4]triazolo[1,5-a]pyrimidin-4(7H)-yl)methyl)benzenesulfonamide

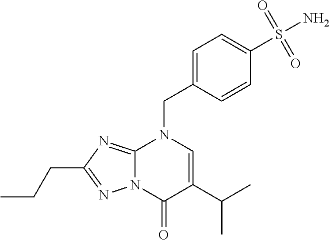 [SMILES: CCCc1nc2n(Cc3ccc(S(N)(=O)=O)cc3)cc(C(C)C)c(=O)n2n1]

### Step 1: 6-Isopropyl-2-propyl-4H-[1,2,4]triazolo[1,5-a]pyrimidin-7-one

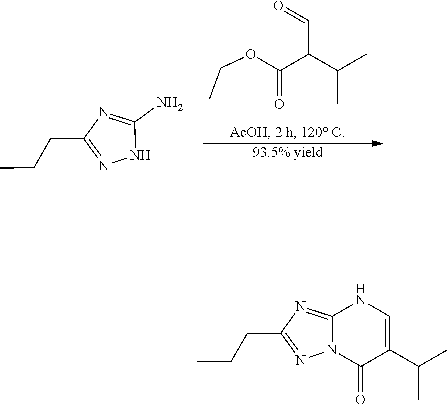

To a stirred solution of 5-propyl-2H-1,2,4-triazol-3-amine (150 mg, 1.19 mmol, 1.0 equiv) was added ethyl 2-formyl-3-methylbutanoate (226 mg, 1.43 mmol, 1.2 equiv) in AcOH (3 mL) at room temperature under nitrogen atmosphere. The resulting mixture was stirred for 2 h at 120° C. under nitrogen atmosphere. The reaction was monitored by LCMS. The mixture was allowed to cool down to 25° C. The product was precipitated by the addition of water. The precipitated solids were collected by filtration and washed with water (2×5 mL), to afford 6-isopropyl-2-propyl-4H-[1,2,4]triazolo[1,5-a]pyrimidin-7-one.

ESI-MS m/z=221.1 [M+H]⁺; Calculated: 220.1.

¹H NMR (400 MHZ, DMSO-d₆) δ 12.93 (s, 1H), 7.71 (d, J=0.7 Hz, 1H), 3.03-2.91 (m, 1H), 2.66 (t, J=7.4 Hz, 2H), 1.76-1.70 (m, 2H), 1.18 (d, J=6.9 Hz, 6H), 0.93 (t, J=7.4 Hz, 3H).

### Step 2: 4-((6-Isopropyl-7-oxo-2-propyl-[1,2,4]triazolo[1,5-a]pyrimidin-4(7H)-yl)methyl)benzenesulfonamide

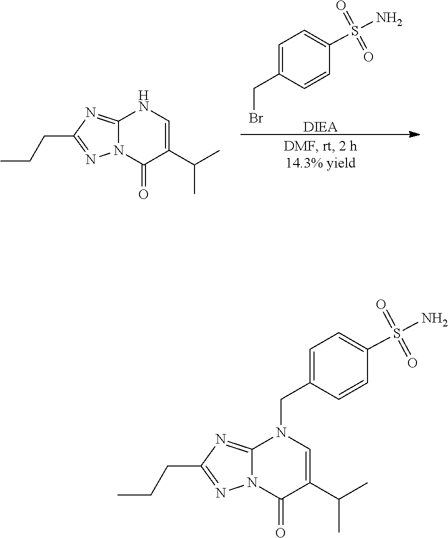 [SMILES: CCCc1nc2[nH]cc(C(C)C)c(=O)n2n1 ; NS(=O)(=O)c1ccc(CBr)cc1 ; CCCc1nc2n(Cc3ccc(S(N)(=O)=O)cc3)cc(C(C)C)c(=O)n2n1]

To a stirred solution of 6-isopropyl-2-propyl-4H-[1,2,4]triazolo[1,5-a]pyrimidin-7-one (200 mg, 0.91 mmol, 1.0 equiv) and 4-(bromomethyl)benzenesulfonamide (273 mg, 1.09 mmol, 1.2 equiv) in DMF (2 mL) was added DIEA (352 mg, 2.72 mmol, 3.0 equiv) at 25° C. under nitrogen atmosphere. The resulting mixture was stirred for 2 h at 120° C. under nitrogen atmosphere. The reaction was monitored by LCMS. The mixture was allowed to cool down to 25° C. The mixture was purified by Prep-HPLC with the following conditions (Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min mL/min; Gradient: 20% B to 35% B in 10 min; Wave Length: 254 nm/220 nm nm; RT1 (min): 9.05) the pure fraction was concentrated then lyophilized to afford 4-((6-isopropyl-7-oxo-2-propyl-[1,2,4]triazolo[1,5-a]pyrimidin-4(7H)-yl)methyl)benzenesulfonamide.

ESI-MS m/z=390.15 [M+H]⁺; Calculated: 389.2

¹H NMR (300 MHZ, DMSO-d₆) δ 8.05 (s, 1H), 7.79 (d, J=8.2 Hz, 2H), 7.53 (d, J=8.2 Hz, 2H), 7.35 (s, 2H), 5.44 (s, 2H), 3.06-2.94 (m, 1H), 2.65 (t, J=7.4 Hz, 2H), 1.78-1.62 (m, 2H), 1.20 (d, J=6.9 Hz, 6H), 0.91 (t, J=7.4 Hz, 3H).

Total proton count from structure: 23.

Total proton count from spectrum: 23

### Compound 29: 5-((2-cyclopropyl-6-isopropyl-7-oxo-[1,2,4]triazolo[1,5-a]pyrimidin-4(7H)-yl)methyl)-6-(methylamino)pyridine-2-sulfonamide

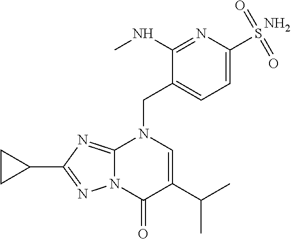

### Step 1: Methyl 6-(benzylsulfanyl)-2-chloropyridine-3-carboxylate

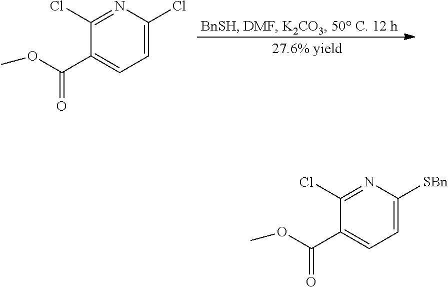

To a stirred solution of methyl 2,6-dichloropyridine-3-carboxylate (30.00 g, 145.6 mmol, 1.0 equiv) and benzyl mercaptan (15.37 g, 123.7 mmol, 0.9 equiv) in DMF (200 mL) were added K₂CO₃ (40.25 g, 291.2 mmol, 2.0 equiv) at 25° C. under nitrogen atmosphere. The resulting mixture was stirred for an additional 12 h at 50° C. The mixture was allowed to cool down to 25° C. The reaction was monitored by LCMS. The resulting mixture was filtered; the filter cake was washed with EtOAc (3×700 mL). The combined organic layers were washed with brine (300 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (0.1% FA), 60% to 80% gradient in 20 min; detector: UV 254 nm. This resulted in methyl 6-(benzylsulfanyl)-2-chloropyridine-3-carboxylate.

ESI-MS m/z=294.02 [M+H]⁺; Calculated: 293.0.

¹H NMR (400 MHZ, DMSO-d₆) δ 8.08 (d, J=8.2 Hz, 1H), 7.49-7.42 (m, 3H), 7.33 (t, J=7.4 Hz, 2H), 7.26 (t, J=7.3 Hz, 1H), 4.45 (s, 2H), 3.85 (s, 3H).

### Step 2: Methyl 2-chloro-6-(chlorosulfonyl)pyridine-3-carboxylate

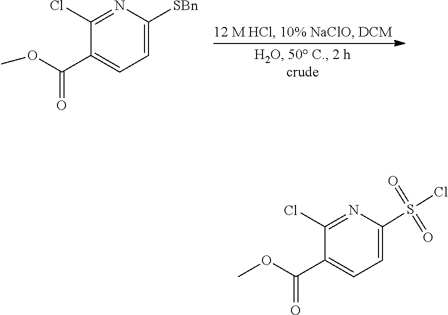

To a stirred solution of methyl 6-(benzylsulfanyl)-2-chloropyridine-3-carboxylate (2×6.00 g, 20.4 mmol, 1.0 equiv) in DCM (180 mL) and H₂O (90 mL) were added HCl (36 mL) and NaClO (180 mL, 10% wt) dropwise at 0° C. under nitrogen atmosphere. The resulting mixture was stirred for an additional 2 h at 50° C. The reaction was monitored by LCMS. To the above mixture was added H₂O (100 mL). The resulting mixture was extracted with DCM (2×300 mL). The combined organic layers were washed with brine (100 mL), and dried over anhydrous Na₂SO₄. After filtration, the filtrate was concentrated under reduced pressure. The crude product was used in the next step directly without further purification.

ESI-MS m/z=268.01 [M−H]⁻; Calculated: 268.9.

### Step 3: Methyl 2-chloro-6-sulfamoylpyridine-3-carboxylate

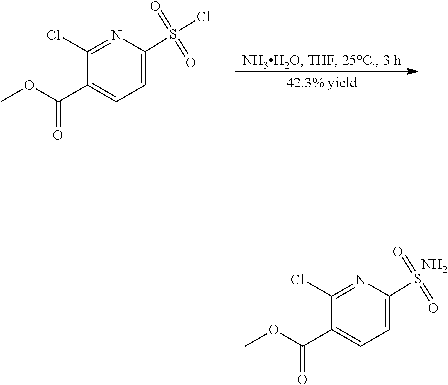

To a stirred solution of methyl 2-chloro-6-(chlorosulfonyl)pyridine-3-carboxylate (8.00 g, 29.6 mmol, 1.0 equiv) was added NH₃·H₂O (14 mL, 88.8 mmol, 3.0 equiv, 25%) in THF (300 mL) at 25° C. under nitrogen atmosphere. The resulting mixture was stirred for an additional 3 h at 25° C. The reaction was monitored by LCMS. The resulting mixture was extracted with DCM/IPA (5:1) (3×150 mL). The combined organic layers were washed with brine (100 mL), and dried over anhydrous Na₂SO₄. After filtration, the filtrate was concentrated under reduced pressure. The product was precipitated by the addition of DCM (50 mL). This resulted in methyl 2-chloro-6-sulfamoylpyridine-3-carboxylate.

ESI-MS m/z=249.01 [M−H]⁻; Calculated: 249.9.

### Step 4: 6-Chloro-5-(hydroxymethyl)pyridine-2-sulfonamide

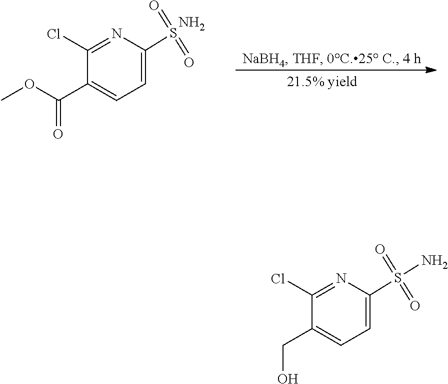

To a stirred solution of methyl 2-chloro-6-sulfamoylpyridine-3-carboxylate (2.40 g, 9.57 mmol, 1.0 equiv) in THF (20 mL) were added NaBH₄ (1.10 g, 28.7 mmol, 3.0 equiv) in portions at 0° C. under nitrogen atmosphere. The resulting mixture was stirred for an additional 4 h at 25° C. The reaction was monitored by LCMS. The reaction was quenched by the addition of Water (10 mL) at 0° C. The resulting mixture was concentrated under reduced pressure. The residue was purified by reversed-phase flash chromatography with the following conditions: column: C18 silica gel; mobile phase: MeCN in Water (0.1% FA), 0% to 10% gradient in 10 min; detector: UV 254 nm. This resulted in 6-chloro-5-(hydroxymethyl)pyridine-2-sulfonamide.

ESI-MS m/z=223.03 [M+H]⁺; Calculated: 221.9.

¹H NMR (400 MHZ, DMSO-d₆) δ 8.17 (d, 1H), 7.97 (d, J=7.8 Hz, 1H), 7.63 (s, 2H), 5.82 (s, 1H), 4.61 (s, 2H).

### Step 5: 5-(Bromomethyl)-6-chloropyridine-2-sulfonamide

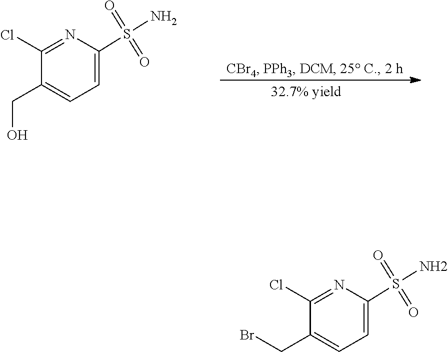 [SMILES: NS(=O)(=O)c1ccc(CO)c(Cl)n1 ; NS(=O)(=O)c1ccc(CBr)c(Cl)n1]

To a stirred solution of 6-chloro-5-(hydroxymethyl)pyridine-2-sulfonamide (450 mg, 2.02 mmol, 1.0 equiv) and PPh₃ (1.06 g, 4.04 mmol, 2.0 equiv) in DCM (6 mL) was added carbon tetrabromide at 25° C. under nitrogen atmosphere. The resulting mixture was stirred for an additional 30 min at 25° C. Into mixture were added CBr₄ (2.01 g, 6.06 mmol, 3.0 equiv). The resulting mixture was stirred for an additional 2 h at 25° C. The reaction was monitored by LCMS. To the above mixture was added H₂O (30 mL). The resulting mixture was extracted with DCM (2×80 mL). The combined organic layers were washed with brine (30 mL), and dried over anhydrous Na₂SO₄. After filtration, the filtrate was concentrated under reduced pressure. The residue was purified by silica gel column chromatography, eluted with PE/EA (3:1) to afford 5-(bromomethyl)-6-chloropyridine-2-sulfonamide.

ESI-MS m/z=283.02&285.03. [M−H]⁻; Calculated: 283.9&285.9.

### Step 6: 6-Chloro-5-({2-cyclopropyl-6-isopropyl-7-oxo-[1,2,4]triazolo[1,5-a]pyrimidin-4-yl}methyl)pyridine-2-sulfonamide

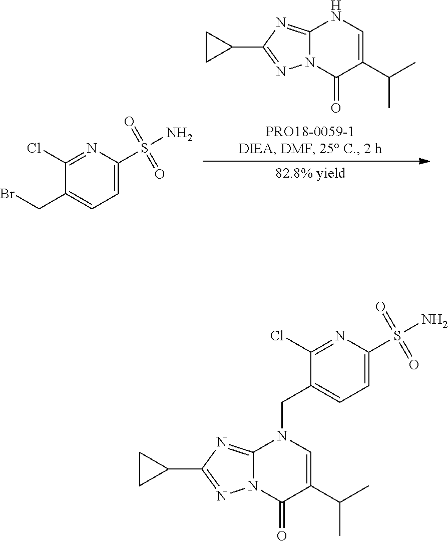 [SMILES: CC(C)c1c[nH]c2nc(C3CC3)nn2c1=O ; NS(=O)(=O)c1ccc(CBr)c(Cl)n1 ; CC(C)c1cn(Cc2ccc(S(N)(=O)=O)nc2Cl)c2nc(C3CC3)nn2c1=O]

To a stirred solution of 5-(bromomethyl)-6-chloropyridine-2-sulfonamide (220 mg, 0.77 mmol, 1.0 equiv) and 2-cyclopropyl-6-isopropyl-4H-[1,2,4]triazolo[1,5-a]pyrimidin-7-one (168 mg, 0.77 mmol, 1.0 equiv) in DMF (2 mL) were added DIEA (0.3 mL, 1.54 mmol, 2.0 equiv) at 25° C. under nitrogen atmosphere. The resulting mixture was stirred for an additional 2 h at 25° C. The reaction was monitored by LCMS. The crude product was purified by Prep-HPLC with the following conditions (Column: XBridge Prep OBD C18 Column, 30*150 mm, 10 μm; Mobile Phase A: Water (10 mmol/L NH₄HCO₃), Mobile Phase B: ACN; Flow rate: 60 mL/min mL/min; Gradient: 32% B to 46% B in 8 min; Wave Length: 254 nm/220 nm nm; RT1 (min): 4) to afford 6-chloro-5-({2-cyclopropyl-6-isopropyl-7-oxo-[1,2,4]triazolo[1,5-a]pyrimidin-4-yl}methyl)pyridine-2-sulfonamide.

ESI-MS m/z=423.02 [M+H]⁺; Calculated: 422.8.

### Step 7: 5-((2-Cyclopropyl-6-isopropyl-7-oxo-[1,2,4]triazolo[1,5-a]pyrimidin-4(7H)-yl)methyl)-6-(methylamino)pyridine-2-sulfonamide

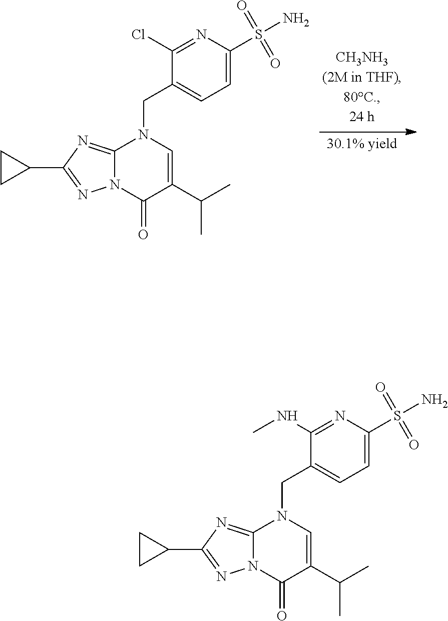 [SMILES: CC(C)c1cn(Cc2ccc(S(N)(=O)=O)nc2Cl)c2nc(C3CC3)nn2c1=O ; CNc1nc(S(N)(=O)=O)ccc1Cn1cc(C(C)C)c(=O)n2nc(C3CC3)nc12]

To a stirred solution of 6-chloro-5-({2-cyclopropyl-6-isopropyl-7-oxo-[1,2,4]triazolo[1,5-a]pyrimidin-4-yl}methyl)pyridine-2-sulfonamide (200 mg, 0.47 mmol, 1.0 equiv) was added CH₃NH₂ (8 mL, 231.0 mmol) (2M in THF) at 25° C. under nitrogen atmosphere. The resulting mixture was stirred for an additional 24 h at 80° C. The mixture was allowed to cool down to 25° C. The reaction was monitored by LCMS. The resulting mixture was diluted with EtOAc (100 mL), The resulting mixture was washed with water (30 mL). The combined organic layers were washed with brine (1×30 mL), and dried over anhydrous Na₂SO₄. After filtration, the filtrate was concentrated under reduced pressure. The crude product was purified by Prep-HPLC with the following conditions (Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 32% B to 46% B in 8 min; Wave Length: 254 nm/220 nm nm; RT1 (min): 4) the pure fraction was concentrated then lyophilized to afford 5-((2-cyclopropyl-6-isopropyl-7-oxo-[1,2,4]triazolo[1,5-a]pyrimidin-4(7H)-yl)methyl)-6-(methylamino)pyridine-2-sulfonamide.

ESI-MS m/z=418.15 [M+H]⁺, as sulfonic acid; Calculated: 417.1.

¹H NMR (400 MHZ, DMSO-d₆) δ 7.93 (s, 1H), 7.27 (d, J=7.5 Hz, 1H), 7.17 (s, 2H), 6.98 (d, J=7.5 Hz, 1H), 6.88 (q, J=4.5 Hz, 1H), 5.16 (s, 2H), 3.06-2.90 (m, 4H), 2.15-1.92 (m, 1H), 1.17 (d, J=6.9 Hz, 6H), 1.06-0.96 (m, 2H), 0.92-0.80 (m, 2H).

Total proton counts from structure: 23.

Total proton counts from spectrum: 23.

### Compound 30:2,6-diisopropyl-4-(4-(S-methylsulfonimidoyl)benzyl)-[1,2,4]triazolo[1,5-a]pyrimidin-7(4H)-one

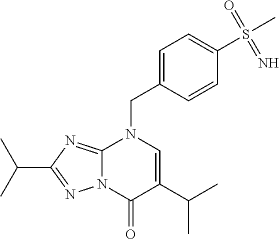 [SMILES: CC(C)c1nc2n(Cc3ccc(S(C)(=N)=O)cc3)cc(C(C)C)c(=O)n2n1]

### Step 1: 2,6-Diisopropyl-4H-[1,2,4]triazolo[1,5-a]pyrimidin-7-one

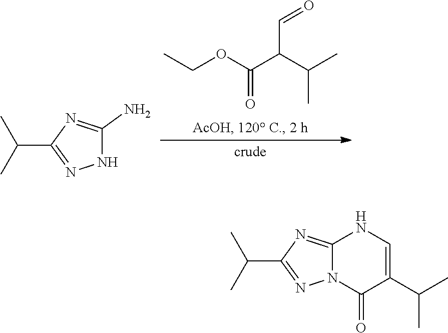

A solution of 5-isopropyl-2H-1,2,4-triazol-3-amine (1.00 g, 7.92 mmol, 1.0 equiv) and ethyl 2-formyl-3-methylbutanoate (1.38 g, 8.72 mmol, 1.1 equiv) in AcOH (10 mL) was stirred for 2 h at 120° C. under nitrogen atmosphere. The reaction was monitored by LCMS. The mixture was allowed to cool down to room temperature. The product was precipitated by the addition of water. The precipitated solids were collected by filtration and washed with water (2×20 mL). This resulted in 2,6-diisopropyl-4H-[1,2,4]triazolo[1,5-a]pyrimidin-7-one (crude).

ESI-MS m/z=221.1 [M+H]⁺; Calculated: 220.1.

¹H NMR (400 MHZ, DMSO-d6) δ 12.93 (s, 1H), 7.72 (s, 1H), 3.09-2.85 (m, 2H), 1.44-1.08 (m, 12H).

### Step 2: 2,6-Diisopropyl-4-(4-(S-methylsulfonimidoyl)benzyl)-[1,2,4]triazolo[1,5-a]pyrimidin-7(4H)-one

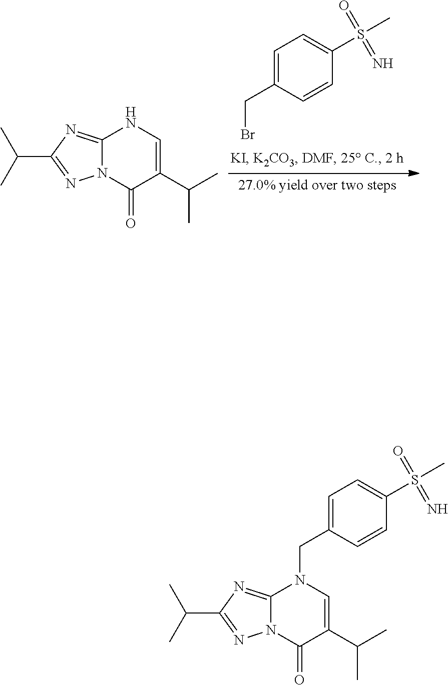 [SMILES: CC(C)c1nc2[nH]cc(C(C)C)c(=O)n2n1 ; CS(=N)(=O)c1ccc(CBr)cc1 ; CC(C)c1nc2n(Cc3ccc(S(C)(=N)=O)cc3)cc(C(C)C)c(=O)n2n1]

A mixture of 2,6-diisopropyl-4H-[1,2,4]triazolo[1,5-a]pyrimidin-7-one (200 mg, 0.91 mmol, 1.0 equiv), [4-(bromomethyl)phenyl](imino)methyl-lambda6-sulfanone (247 mg, 0.99 mmol, 1.1 equiv), KI (150 mg, 0.91 mmol, 1.0 equiv) and K₂CO₃ (376 mg, 2.72 mmol, 3.0 equiv) in DMF (2 mL) was stirred for 2 h at 25° C. under nitrogen atmosphere. The reaction was monitored by LCMS. The resulting mixture was diluted with EtOAc (10 mL). The resulting mixture was washed with 5×10 ml of water. The resulting mixture was concentrated under reduced pressure. The residue was purified by Prep-HPLC with the following conditions: (Column: Sunfire prep C18 column 30*150 mm, 5 m; Mobile Phase A: Water (0.1% FA), Mobile Phase B: ACN; Flow rate: 60 mL/min; Gradient: 15% B to 34% B in 10 min; Wave Length: 254 nm/220 nm nm; RT1 (min): 9.87,), the pure fraction was concentrated then lyophilized to afford 2,6-diisopropyl-4-(4-(S-methylsulfonimidoyl)benzyl)-[1,2,4]triazolo[1,5-a]pyrimidin-7(4H)-one.

ESI-MS m/z=388.25 [M+H]⁺; Calculated: 387.2.

¹H NMR (400 MHz, DMSO-d6) δ 8.07 (s, 1H), 7.91 (d, J=8.2 Hz, 2H), 7.60 (d, J=8.1 Hz, 2H), 5.46 (s, 2H), 4.21 (s, 1H), 3.04 (s, 3H), 3.03-2.95 (m, 2H), 1.27 (d, J=6.9 Hz, 6H), 1.19 (d, J=6.9 Hz, 6H).

Total proton count from structure: 25.

Total proton count from spectrum: 25.

### Compound 31 Rac: 2-ethyl-6-isopropyl-4-(4-(S-methylsulfonimidoyl)benzyl)-[1,2,4]triazolo[1,5-a]pyrimidin-7(4H)-one

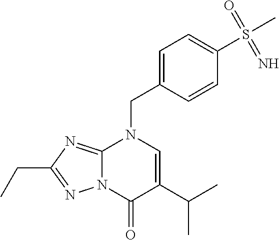 [SMILES: CCc1nc2n(Cc3ccc(S(C)(=N)=O)cc3)cc(C(C)C)c(=O)n2n1]

### Step 1: 2-Ethyl-6-isopropyl-4-(4-(S-methylsulfonimidoyl)benzyl)-[1,2,4]triazolo[1,5-a]pyrimidin-7(4H)-one

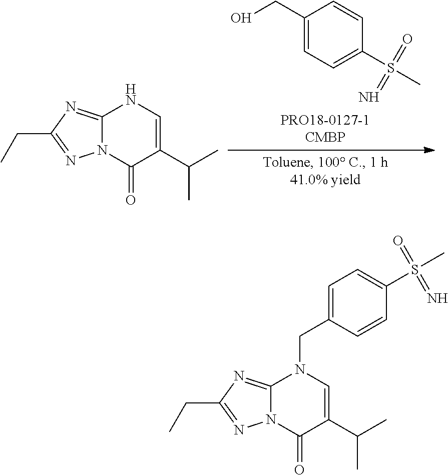 [SMILES: CS(=N)(=O)c1ccc(CO)cc1 ; CCc1nc2[nH]cc(C(C)C)c(=O)n2n1 ; CCc1nc2n(Cc3ccc(S(C)(=N)=O)cc3)cc(C(C)C)c(=O)n2n1]

To a stirred mixture of 2-ethyl-6-isopropyl-4H-[1,2,4]triazolo[1,5-a]pyrimidin-7-one (150 mg, 0.72 mmol, 1.0 equiv) and [4-(hydroxymethyl)phenyl](imino)methyl-lambda6-sulfanone (148 mg, 0.80 mmol, 1.1 equiv) in toluene (3 mL) was added CMBP (526 mg, 2.18 mmol, 3.0 equiv) at 25° C. under nitrogen atmosphere. The resulting mixture was stirred for 1 h at 100° C. under nitrogen atmosphere. The desired product could be detected by LCMS. The resulting mixture was concentrated under reduced pressure. The residue was purified by reversed-phase flash chromatography with the following conditions: column: C18; mobile phase: MeCN in Water (10 mmol/L NH₄HCO₃), 17% to 45% gradient in 25 min; detector: UV 220 nm to afford the crude product. The crude product was purified by Prep-HPLC with the following conditions (Column: Kinetex EVO C18 Column, 30+150, 5 μm; Mobile Phase A: Water (0.1% FA), Mobile Phase B: MEOH; Flow rate: 60 mL/min mL/min; Gradient: isocratic Wave Length: 220/250 nm; RT1 (min): 8.07), the pure fraction was concentrated under reduced pressure then lyophilized to afford 2-ethyl-6-isopropyl-4-(4-(S-methylsulfonimidoyl)benzyl)-[1,2,4]triazolo[1,5-a]pyrimidin-7(4H)-one.

ESI-MS m/z=374.05 [M+H]⁺; Calculated: 373.2.

¹H NMR (400 MHZ, DMSO-d₆) δ 8.09 (s, 1H), 7.96-7.89 (m, 2H), 7.62-7.55 (m, 2H), 5.48 (s, 2H), 4.23 (s, 1H), 3.06 (s, 3H), 3.05-2.96 (m, 1H), 2.76-2.66 (m, 2H), 1.29-1.24 (m, 3H), 1.22-1.19 (m, 6H).

Total proton count from structure: 23.

Total proton count from spectrum: 23.

### Compound 31A: rel-(S)-2-ethyl-6-isopropyl-4-(4-(S-methylsulfonimidoyl)benzyl)-[1,2,4]triazolo[1,5-a]pyrimidin-7(4H)-one

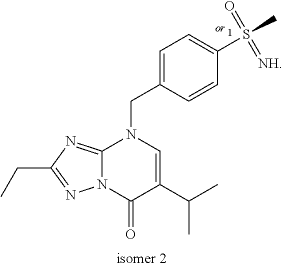 [SMILES: CCc1nc2n(Cc3ccc([S@@](C)(=N)=O)cc3)cc(C(C)C)c(=O)n2n1]

### Step 1: Rel-(S)-2-ethyl-6-isopropyl-4-(4-(S-methylsulfonimidoyl)benzyl)-[1,2,4]triazolo[1,5-a]pyrimidin-7(4H)-one

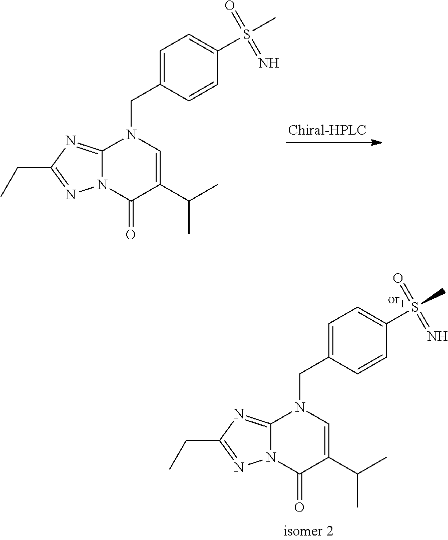

The racemate (106 mg) was separated by Chiral-HPLC with the following conditions (Column: CHIRAL ART Cellulose-SC, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 200/220 nm; RT1 (min): 13.973; RT2 (min): 17.498; Sample Solvent: ETOH: DCM=1:1; Injection Volume: 0.5 mL; Number Of Runs: 3), the pure fractions was concentrated and then lyophilized respectively to afford Rel-(S)-2-ethyl-6-isopropyl-4-(4-(S-methylsulfonimidoyl)benzyl)-[1,2,4]triazolo[1,5-a]pyrimidin-7(4H)-one.

ESI-MS m/z=374.05 [M+H]⁺; Calculated: 373.2.

¹H NMR (400 MHZ, DMSO-d₆) δ 8.07 (s, 1H), 7.94-7.88 (m, 2H), 7.60-7.53 (m, 2H), 5.47 (s, 2H), 4.21 (s, 1H), 3.07-3.03 (m, 3H), 3.03-2.93 (m, 1H), 2.75-2.65 (m, 2H), 1.28-1.20 (m, 3H), 1.23-1.17 (m, 6H).

Total proton count from structure: 23.

Total proton count from spectrum: 23.

### Compound 31B: rel-(R)-2-ethyl-6-isopropyl-4-(4-(S-methylsulfonimidoyl)benzyl)-[1,2,4]triazolo[1,5-a]pyrimidin-7(4H)-one

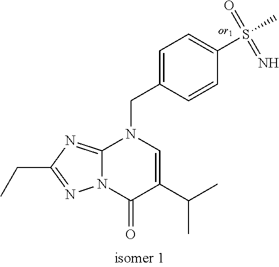 [SMILES: CCc1nc2n(Cc3ccc([S@](C)(=N)=O)cc3)cc(C(C)C)c(=O)n2n1]

### Step 1: Rel-(R)-2-ethyl-6-isopropyl-4-(4-(S-methylsulfonimidoyl)benzyl)-[1,2,4]triazolo[1,5-a]pyrimidin-7(4H)-one

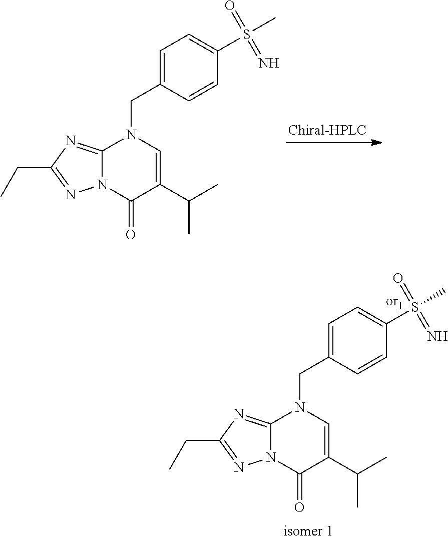

The racemate (106 mg) was separated by Chiral-HPLC with the following conditions (Column: CHIRAL ART Cellulose-SC, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 200/220 nm; RT1 (min): 13.973; RT2 (min): 17.498; Sample Solvent: ETOH: DCM=1:1; Injection Volume: 0.5 mL; Number Of Runs: 3), the pure fractions was concentrated and then lyophilized respectively to afford rel-(R)-2-ethyl-6-isopropyl-4-(4-(S-methylsulfonimidoyl)benzyl)-[1,2,4]triazolo[1,5-a]pyrimidin-7(4H)-one.

ESI-MS m/z=374.10 [M+H]⁺; Calculated: 373.2.

¹H NMR (400 MHZ, DMSO-d₆) δ 8.07 (s, 1H), 7.95-7.88 (m, 2H), 7.61-7.54 (m, 2H), 5.47 (s, 2H), 4.22 (s, 1H), 3.05 (s, 3H), 3.04-2.95 (m, 1H), 2.75-2.65 (m, 2H), 1.27-1.16 (m, 9H).

Total proton count from structure: 23.

Total proton count from spectrum: 23.

### Compound 32 Rac: 6-isopropyl-4-(4-(S-methylsulfonimidoyl)benzyl)-2-(2,2,2-trifluoroethyl)-[1,2,4]triazolo[1,5-a]pyrimidin-7(4H)-one

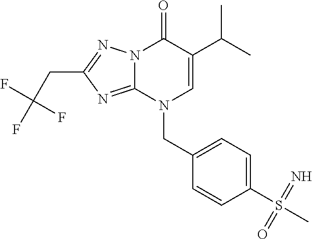 [SMILES: CC(C)c1cn(Cc2ccc(S(C)(=N)=O)cc2)c2nc(CC(F)(F)F)nn2c1=O]

### Step 1: 3,3,3-Trifluoropropanoyl chloride

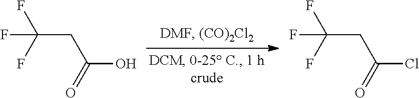 [SMILES: O=C(O)CC(F)(F)F ; O=C(Cl)CC(F)(F)F]

To a stirred solution of 3,3,3-trifluoropropanoic acid (60.00 g, 468.6 mmol, 1.0 equiv) and DMF (3.42 g, 46.9 mmol, 0.10 equiv) in DCM (600 mL) was added (CO)₂Cl₂ (200 mL, 2.34 mol, 5.0 equiv) dropwise at 0° C. under nitrogen atmosphere. The resulting mixture was stirred for 1 h at 25° C. under nitrogen atmosphere. The resulting mixture was concentrated under reduced pressure to afford 3,3,3-trifluoropropanoyl chloride (crude).

### Step 2: 2-(3,3,3-Trifluoropropanoyl) hydrazine-1-carboximidamide

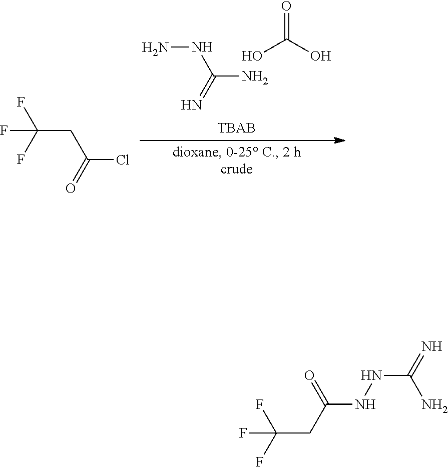

To a stirred solution of aminoguanidine; carbonic acid (111.50 g, 819.2 mmol, 2.0 equiv) and TBAB (13.20 g, 41.0 mmol, 0.10 equiv) in dioxane (600 mL) was added 3,3,3-trifluoropropanoyl chloride (60.00 g, 409.6 mmol, 1.0 equiv) dropwise at 0° C. under nitrogen atmosphere. The resulting mixture was stirred for 2 h at 25° C. under nitrogen atmosphere. The reaction was monitored by LCMS. The resulting mixture was concentrated under reduced pressure. The resulting mixture was diluted with water (300 mL). The mixture basified to pH 12 with 1 M NaOH(aq.). The resulting mixture was filtered, the filter cake was washed with water (2×30 mL). The filtrate (600 mL) was used in the next step directly without further purification.

ESI-MS m/z=184.95 [M+H]⁺; Calculated: 184.1.

### Step 3: 3-(2,2,2-Trifluoroethyl)-1H-1,2,4-triazol-5-amine

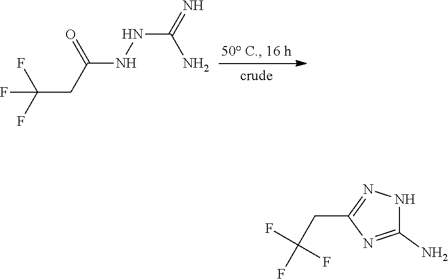 [SMILES: N=C(N)NNC(=O)CC(F)(F)F ; Nc1nc(CC(F)(F)F)n[nH]1]

The residue (600 mL) was basified to pH 12 with 1M NaOH(aq.). The mixture was stirred for 16 h at 50° C. under nitrogen atmosphere. The reaction was monitored by LCMS. The mixture was allowed to cool down to 0° C. The mixture was neutralized to pH 7 with 1M HCl (aq.). The resulting mixture was extracted with EtOAc (3×500 mL). The combined organic layers were washed with brine (1×1500 mL), and dried over anhydrous Na₂SO₄. After filtration, the filtrate was concentrated under reduced pressure. This resulted in 3-(2,2,2-trifluoroethyl)-1H-1,2,4-triazol-5-amine (crude).

ESI-MS m/z=166.95 [M+H]⁺; Calculated: 166.0.

### Step 4: 6-Isopropyl-2-(2,2,2-trifluoroethyl)-[1,2,4]triazolo[1,5-a]pyrimidin-7(4H)-one

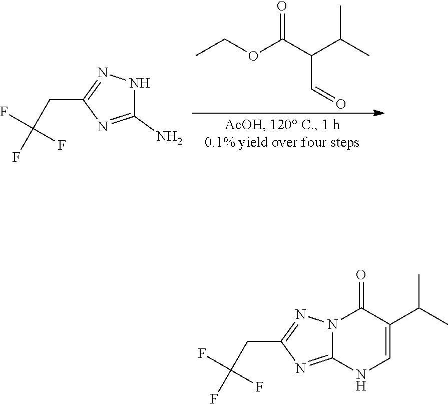 [SMILES: Nc1nc(CC(F)(F)F)n[nH]1 ; CCOC(=O)C(C=O)C(C)C ; CC(C)c1c[nH]c2nc(CC(F)(F)F)nn2c1=O]

A mixture of 3-(2,2,2-trifluoroethyl)-1H-1,2,4-triazol-5-amine (500 mg, 3.01 mmol, 1.0 equiv) and ethyl 2-formyl-3-methylbutanoate (476 mg, 3.01 mmol, 1.0 equiv) in AcOH (5 mL) was stirred for 1 h at 120° C. under nitrogen atmosphere. The desired product could be detected by LCMS. The mixture was allowed to cool down to 25° C. The resulting mixture was concentrated under reduced pressure. The resulting mixture was diluted with water (50 mL). The resulting mixture was extracted with EtOAc (3×50 mL). The combined organic layers were washed with brine (2×200 mL), and dried over anhydrous Na₂SO₄. After filtration, the filtrate was concentrated under reduced pressure. The residue was purified by silica gel column chromatography, eluted with EA/PE (70% to 100% gradient in 20 min) to afford 6-isopropyl-2-(2,2,2-trifluoroethyl)-[1,2,4]triazolo[1,5-a]pyrimidin-7(4H)-one.

ESI-MS m/z=261.00 [M+H]⁺; Calculated: 260.1.

¹H NMR (400 MHz, DMSO-d₆) δ 13.18 (s, 1H), 7.80 (s, 1H), 3.97-3.84 (m, 2H), 3.04-2.94 (m, 1H), 1.19 (d, J=6.9 Hz, 6H).

¹⁹F NMR (377 MHZ, DMSO-d₆) δ −62.74.

### Step 5: 6-Isopropyl-4-(4-(S-methylsulfonimidoyl)benzyl)-2-(2,2,2-trifluoroethyl)-[1,2,4]triazolo[1,5-a]pyrimidin-7(4H)-one

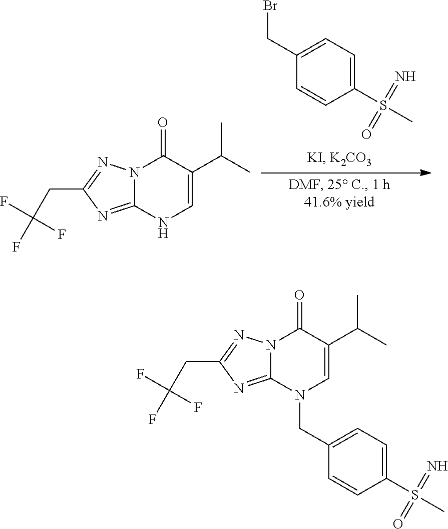 [SMILES: CC(C)c1c[nH]c2nc(CC(F)(F)F)nn2c1=O ; CS(=N)(=O)c1ccc(CBr)cc1 ; CC(C)c1cn(Cc2ccc(S(C)(=N)=O)cc2)c2nc(CC(F)(F)F)nn2c1=O]

To a stirred mixture of 6-isopropyl-2-(2,2,2-trifluoroethyl)-[1,2,4]triazolo[1,5-a]pyrimidin-7(4H)-one (150 mg, 0.58 mmol, 1.0 equiv) and K₂CO₃ (239 mg, 1.73 mmol, 3.0 equiv) in DMF (6 mL) were added (4-(bromomethyl)phenyl)(imino)(methyl)-l6-sulfanone (172 mg, 0.69 mmol, 1.2 equiv) and KI (191 mg, 1.15 mmol, 2.0 equiv) at 25° C. The resulting mixture was stirred for 1 h at 25° C. under nitrogen atmosphere. The desired product could be detected by LCMS. The resulting mixture was diluted with water (100 mL). The resulting mixture was extracted with EtOAc (3×100 mL). The combined organic layers were washed with brine (1×300 mL), and dried over anhydrous Na₂SO₄. After filtration, the filtrate was concentrated under reduced pressure. The crude product (200 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 11% B to 31% B in 10 min; Wave Length: 254 nm/220 nm; RT1 (min): 9.87), the pure fraction was concentrated then lyophilized to afford 6-isopropyl-4-(4-(S-methylsulfonimidoyl)benzyl)-2-(2,2,2-trifluoroethyl)-[1,2,4]triazolo[1,5-a]pyrimidin-7(4H)-one.

ESI-MS m/z=428.15 [M+H]⁺; Calculated: 427.1.

¹H NMR (400 MHZ, DMSO-d₆) δ 8.15 (s, 1H), 7.92 (d, J=8.3 Hz, 2H), 7.60 (d, J=8.4 Hz, 2H), 5.51 (s, 2H), 4.24 (s, 1H), 4.00-3.87 (m, 2H), 3.06 (s, 3H), 3.05-2.98 (m, 1H), 1.22 (d, J=6.9 Hz, 6H).

¹⁹F NMR (377 MHZ, DMSO-d₆) δ −62.73.

Total proton count from structure: 20.

Total proton count from spectrum: 20.

### Compound 32A: rel-(R)-6-isopropyl-4-(4-(S-methylsulfonimidoyl)benzyl)-2-(2,2,2-trifluoroethyl)-[1,2,4]triazolo[1,5-a]pyrimidin-7(4H)-one

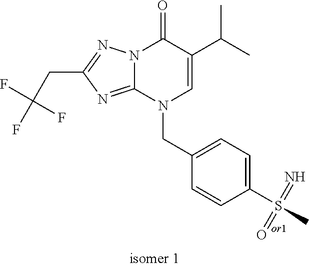 [SMILES: CC(C)c1cn(Cc2ccc([S@](C)(=N)=O)cc2)c2nc(CC(F)(F)F)nn2c1=O]

### Step 1: Rel-(R)-6-isopropyl-4-(4-(S-methylsulfonimidoyl)benzyl)-2-(2,2,2-trifluoroethyl)-[1,2,4]triazolo[1,5-a]pyrimidin-7(4H)-one

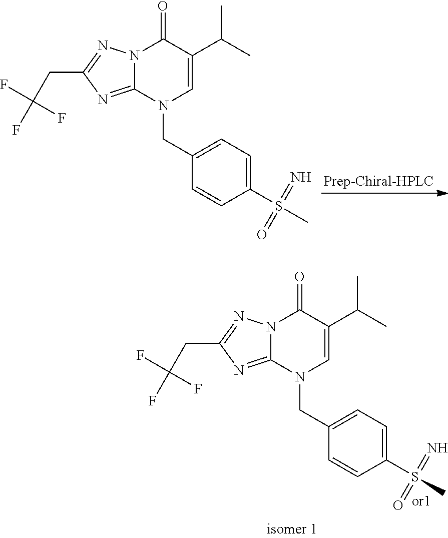 [SMILES: CC(C)c1cn(Cc2ccc(S(C)(=N)=O)cc2)c2nc(CC(F)(F)F)nn2c1=O ; CC(C)c1cn(Cc2ccc([S@](C)(=N)=O)cc2)c2nc(CC(F)(F)F)nn2c1=O]

The racemate (94 mg) was separated by Prep-Chiral-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 202/220 nm; RT1 (min): 26; Sample Solvent: MeOH: DCM=3:1-HPLC; Number Of Runs: 2), the pure factions were concentrated under vacuum then lyophilized to afford rel-(R)-6-isopropyl-4-(4-(S-methylsulfonimidoyl)benzyl)-2-(2,2,2-trifluoroethyl)-[1,2,4]triazolo[1,5-a]pyrimidin-7(4H)-one.

ESI-MS m/z=428.00 [M+H]⁺; Calculated: 427.1.

¹H NMR (400 MHZ, DMSO-d₆) δ 8.13 (d, J=0.8 Hz, 1H), 7.95-7.85 (m, 2H), 7.62-7.51 (m, 2H), 5.48 (s, 2H), 4.22 (s, 1H), 4.02-3.82 (m, 2H), 3.07-2.97 (m, 4H), 1.21 (d, J=7.0 Hz, 6H).

¹⁹F NMR (377 MHZ, DMSO-d₆) δ −62.70.

Total proton count from structure: 20.

Total proton count from spectrum: 20.

### Compound 32B: rel-(S)-6-isopropyl-4-(4-(S-methylsulfonimidoyl)benzyl)-2-(2,2,2-trifluoroethyl)-[1,2,4]triazolo[1,5-a]pyrimidin-7(4H)-one

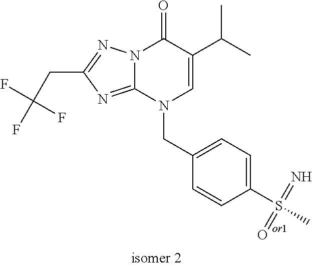 [SMILES: CC(C)c1cn(Cc2ccc([S@@](C)(=N)=O)cc2)c2nc(CC(F)(F)F)nn2c1=O]

### Step 1: Rel-(S)-6-isopropyl-4-(4-(S-methylsulfonimidoyl)benzyl)-2-(2,2,2-trifluoroethyl)-[1,2,4]triazolo[1,5-a]pyrimidin-7(4H)-one

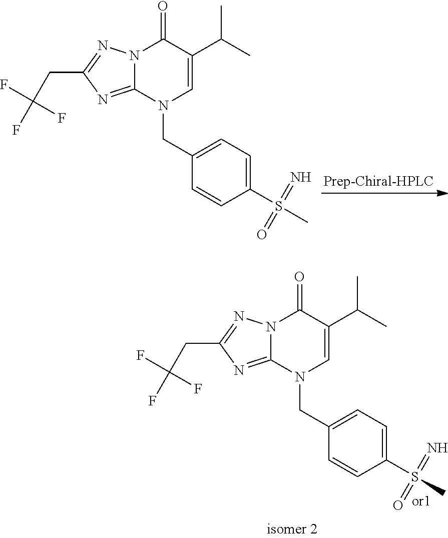 [SMILES: CC(C)c1cn(Cc2ccc(S(C)(=N)=O)cc2)c2nc(CC(F)(F)F)nn2c1=O ; CC(C)c1cn(Cc2ccc([S@](C)(=N)=O)cc2)c2nc(CC(F)(F)F)nn2c1=O]

The racemate (94 mg) was separated by Prep-Chiral-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 202/220 nm; RT2 (min): 34.9; Sample Solvent: MeOH: DCM=3:1-HPLC; Number Of Runs: 2), the pure factions were concentrated under vacuum then lyophilized to afford rel-(S)-6-isopropyl-4-(4-(S-methylsulfonimidoyl)benzyl)-2-(2,2,2-trifluoroethyl)-[1,2,4]triazolo[1,5-a]pyrimidin-7(4H)-one.

ESI-MS m/z=428.05 [M+H]⁺; Calculated: 427.1.

¹H NMR (400 MHZ, DMSO-d₆) δ 8.15-8.10 (m, 1H), 7.93-7.87 (m, 2H), 7.63-7.50 (m, 2H), 5.48 (s, 2H), 4.22 (s, 1H), 4.03-3.78 (m, 2H), 3.12-2.94 (m, 4H), 1.21 (d, J=6.9 Hz, 6H).

¹⁹F NMR (377 MHZ, DMSO-d₆) δ −62.70.

Total proton count from structure: 20.

Total proton count from spectrum: 20.

### Compound 33: 6-isopropyl-2-(methoxymethyl)-4-(4-(S-methylsulfonimidoyl)benzyl)-[1,2,4]triazolo[1,5-a]pyrimidin-7(4H)-one

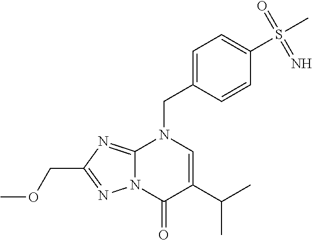

### Step 1: 6-Isopropyl-2-(methoxymethyl)-[1,2,4]triazolo[1,5-a]pyrimidin-7(4H)-one

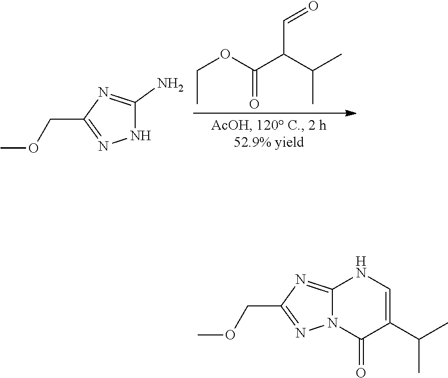

A solution of 5-(methoxymethyl)-2H-1,2,4-triazol-3-amine (300 mg, 2.34 mmol, 1.0 equiv) and ethyl 2-formyl-3-methylbutanoate (407 mg, 2.58 mmol, 1.1 equiv) in AcOH (3 mL) was stirred for 2 h at 120° C. under nitrogen atmosphere. The desired product could be detected by LCMS. The mixture was allowed to cool down to 25° C. and purified by trituration with EtOAc (2 mL) to afford 6-isopropyl-2-(methoxymethyl)-[1,2,4]triazolo[1,5-a]pyrimidin-7(4H)-one.

ESI-MS m/z=223.00 [M+H]⁺; Calculated: 222.1.

### Step 2: 6-Isopropyl-2-(methoxymethyl)-4-(4-(S-methylsulfonimidoyl)benzyl)-[1,2,4]triazolo[1,5-a]pyrimidin-7(4H)-one

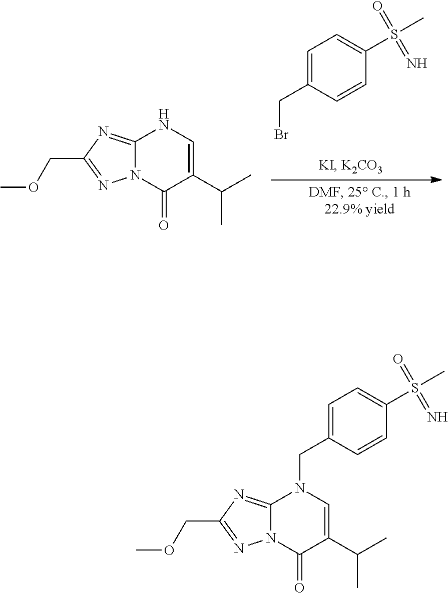

To a stirred solution of 6-isopropyl-2-(methoxymethyl)-[1,2,4]triazolo[1,5-a]pyrimidin-7(4H)-one (230 mg, 1.04 mmol, 1.0 equiv) and (4-(bromomethyl)phenyl)(imino)(methyl)-l6-sulfanone (385 mg, 1.55 mmol, 1.5 equiv) in DMF (2 mL) were added KI (172 mg, 1.04 mmol, 1.0 equiv) and K₂CO₃ (286 mg, 2.07 mmol, 2.0 equiv) at 25° C. The resulting mixture was stirred for 1 h at 25° C. under nitrogen atmosphere. The desired product could be detected by LCMS. The resulting mixture was diluted with water (100 mL). The mixture was basified to pH 12 with NaOH(aq.). The resulting mixture was extracted with CHCl3/IPA (3×100 mL). The combined organic layers were washed with brine (200 mL), and dried over anhydrous Na₂SO₄. After filtration, the filtrate was concentrated under reduced pressure. The crude product (150 mg) was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min mL/min; Gradient: 5% B to 32% B in 7 min; Wave Length: 254 nm/220 nm nm; RT1 (min): 6.77), the pure fraction was concentrated then lyophilized to afford 6-isopropyl-2-(methoxymethyl)-4-(4-(S-methylsulfonimidoyl)benzyl)-[1,2,4]triazolo[1,5-a]pyrimidin-7(4H)-one.

ESI-MS m/z=390.10 [M+H]⁺; Calculated: 389.2.

¹H NMR (400 MHZ, DMSO-d₆) δ 8.15-8.05 (br, 1H), 7.94-7.86 (m, 2H), 7.59-7.52 (m, 2H), 5.49 (s, 2H), 4.46 (s, 2H), 4.25 (s, 1H), 3.31 (s, 3H), 3.07-2.96 (m, 4H), 1.24-1.16 (m, 6H).

Total proton count from structure: 23.

Total proton count from spectrum: 23.

### Compound 34: (1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-sulfonamide

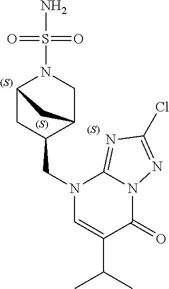

### Step 1: 2-Amino-6-isopropyl-[1,2,4]triazolo[1,5-a]pyrimidin-7(4H)-one

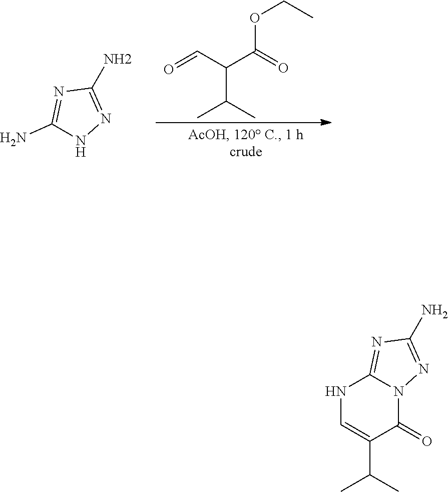

A solution of 1H-1,2,4-triazole-3,5-diamine (1.00 g, 10.1 mmol, 1.0 equiv) and methyl 2-formyl-3-methylbutanoate (1.60 g, 11.1 mmol, 1.1 equiv) in AcOH (10 mL) was stirred for 2 h at 120° C. under nitrogen atmosphere. The desired product could be detected by LCMS. The mixture was allowed to cool down to 25° C. The residue was purified by trituration with water (5 mL) to afford 2-amino-6-isopropyl-[1,2,4]triazolo[1,5-a]pyrimidin-7(4H)-one (crude).

ESI-MS m/z=192.10 [M−H]⁻; Calculated: 193.1.

### Step 2: 2-Chloro-6-isopropyl-[1,2,4]triazolo[1,5-a]pyrimidin-7(4H)-one

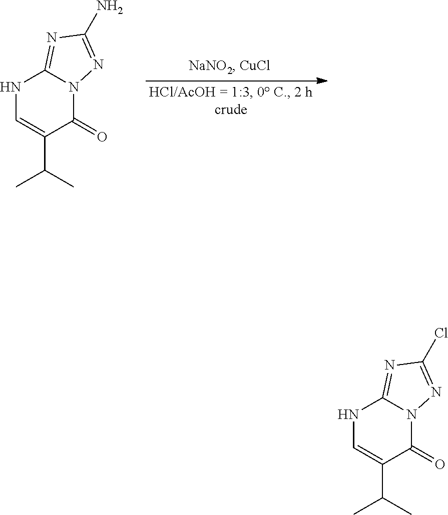

To a stirred solution of 2-amino-6-isopropyl-[1,2,4]triazolo[1,5-a]pyrimidin-7(4H)-one (2.00 g, 10.4 mmol, 1.0 equiv) in HCl (10 mL) and AcOH (30 mL) was added NaNO₂ (3.57 g, 51.8 mmol, 5.0 equiv) and CuCl (2.05 g, 20.7 mmol, 2.0 equiv) in portions at 0° C. The resulting mixture was stirred for 2 h at 0° C. under nitrogen atmosphere. The desired product could be detected by LCMS. The resulting mixture was diluted with water (200 mL). The resulting mixture was extracted with CH₂Cl₂/IPA=3:1 (3×200 mL). The combined organic layers were washed with brine (500 mL), and dried over anhydrous Na₂SO₄. After filtration, the filtrate was concentrated under reduced pressure. The residue was purified by trituration with EtOAc (20 mL) to afford 2-chloro-6-isopropyl-[1,2,4]triazolo[1,5-a]pyrimidin-7(4H)-one (crude).

ESI-MS m/z=213.10 [M+H]⁺; Calculated: 212.0.

### Step 3: Tert-butyl(1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-carboxylate

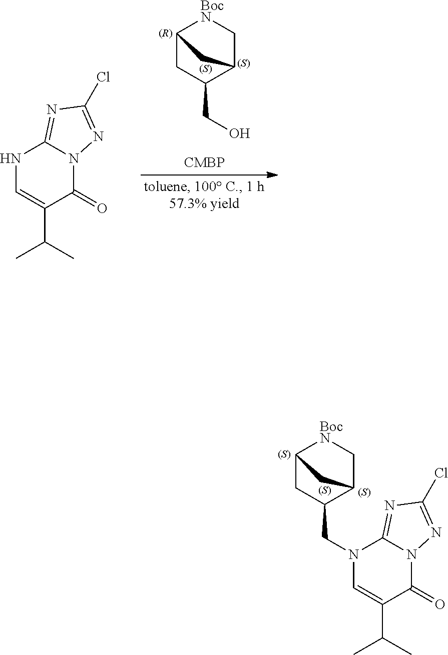

To a stirred solution of 2-chloro-6-isopropyl-[1,2,4]triazolo[1,5-a]pyrimidin-7(4H)-one (309 mg, 1.45 mmol, 1.1 equiv) and tert-butyl(1R,4S,5S)-5-(hydroxymethyl)-2-azabicyclo[2.2.1]heptane-2-carboxylate (300 mg, 1.32 mmol, 1.0 equiv) in Toluene (6 mL) was added 2-(tributyl-lambda5-phosphanylidene) acetonitrile (1.59 g, 6.61 mmol, 5.0 equiv) at 25° C. The resulting mixture was stirred for 1 h at 100° C. under nitrogen atmosphere. The desired product could be detected by LCMS. The mixture was allowed to cool down to 25° C. The resulting mixture was concentrated under reduced pressure. The residue was purified by reversed-phase flash chromatography with the following conditions: column: C18 silica gel; mobile phase: MeCN in Water (10 mmol/L NH₄HCO₃), 0% to 40% gradient in 10 min; detector: UV 254 nm to afford the crude product. The crude product (400 mg) was purified by silica gel column chromatography, eluted with PE/EA (3:1) to afford tert-butyl(1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-carboxylate.

ESI-MS m/z=422.10 [M+H]⁺; Calculated: 421.2.

¹H NMR (400 MHZ, DMSO-d₆) δ 7.99 (s, 1H), 4.12-3.99 (m, 2H), 3.98-3.90 (m, 1H), 3.17-3.06 (m, 1H), 3.04-2.95 (m, 1H), 2.83 (d, J=9.6 Hz, 1H), 2.36-2.25 (m, 2H), 1.76-1.64 (m, 2H), 1.59-1.47 (m, 1H), 1.40-1.32 (m, 9H), 1.25-1.18 (m, 7H).

### Step 4: 4-(((1R,4S,5S)-2-azabicyclo[2.2.1]heptan-5-yl)methyl)-2-chloro-6-isopropyl-[1,2,4]triazolo[1,5-a]pyrimidin-7(4H)-one HCl salt

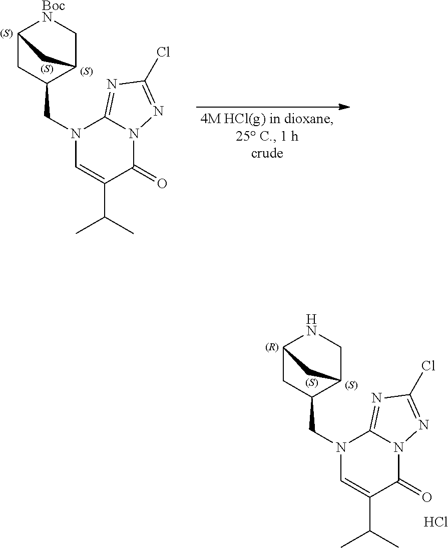

A solution of tert-butyl(1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-carboxylate (200 mg, 0.47 mmol, 1.0 equiv) in 4M HCl (gas) in 1,4-dioxane (5 mL) was stirred for 1 h at 25° C. The desired product could be detected by LCMS. The resulting mixture was concentrated under reduced pressure to afford 4-(((1R,4S,5S)-2-azabicyclo[2.2.1]heptan-5-yl)methyl)-2-chloro-6-isopropyl-[1,2,4]triazolo[1,5-a]pyrimidin-7(4H)-one HCl salt (crude).

ESI-MS m/z=322.00 [M+H]⁺; Calculated: 321.1.

### Step 5: (1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-sulfonamide

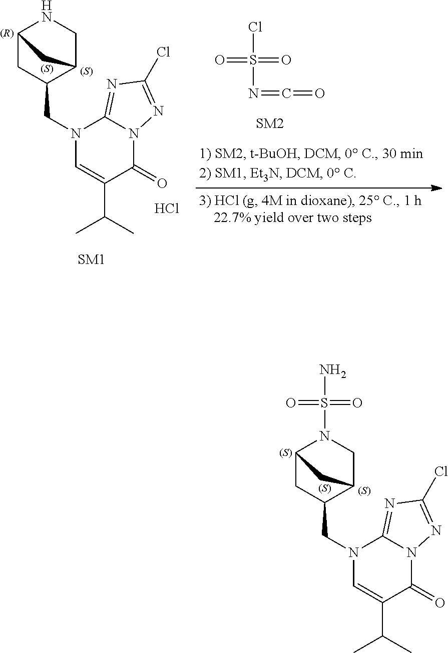

To a stirred solution of chlorosulfonyl isocyanate (284 mg, 2.01 mmol, 6.0 equiv) in DCM (1.7 mL) was added t-BuOH (223 mg, 3.02 mmol, 9.0 equiv) dropwise at 0° C. The resulting mixture was stirred for 30 min at 0° C. under nitrogen atmosphere. To a stirred solution of 4-(((1R,4S,5S)-2-azabicyclo[2.2.1]heptan-5-yl)methyl)-2-chloro-6-isopropyl-[1,2,4]triazolo[1,5-a]pyrimidin-7(4H)-one HCl salt (120 mg, 0.34 mmol, 1.0 equiv) in DCM (3 mL) was added Et₃N (339 mg, 3.35 mmol, 10.0 equiv) for 5 min at 25° C. under nitrogen atmosphere followed by the addition of above mixture dropwise at 0° C. The resulting mixture was stirred for 1 h at 0° C. under nitrogen atmosphere. The desired product could be detected by LCMS. The resulting mixture was concentrated under reduced pressure. To the above mixture was added 4M HCl (gas) in 1,4-dioxane (5 mL) over 1 min at 25° C. The resulting mixture was stirred for an additional 1 h at 25° C. The desired product could be detected by LCMS. The resulting mixture was concentrated under reduced pressure. The resulting mixture was diluted with water (100 mL). The mixture was basified to pH 8 with saturated Na₂CO₃ (aq.). The resulting mixture was extracted with CH₂Cl₂/IPA (3×100 mL). The combined organic layers were washed with brine (200 mL), and dried over anhydrous Na₂SO₄. After filtration, the filtrate was concentrated under reduced pressure. The crude product (120 mg) was purified by Prep-HPLC with the following conditions (Column: XSelect CSH Prep C18 OBD Column, 30*150 mm, 5 m; Mobile Phase A: Water (0.1% FA), Mobile Phase B: ACN; Flow rate: 60 mL/min; Gradient: 14% B to 34% B in 9 min; Wave Length: 254 nm/220 nm nm; RT1 (min): 9.53), the pure fraction was concentrated then lyophilized to afford (1S,4S,5S)-5-((2-chloro-6-isopropyl-7-oxo-[1,2,4]triazolo[1,5-a]pyrimidin-4(7H)-yl)methyl)-2-azabicyclo[2.2.1]heptane-2-sulfonamide.

ESI-MS m/z=401.05 [M+H]⁺; Calculated: 400.1.

¹H NMR (400 MHZ, DMSO-d₆) δ 7.99 (s, 1H), 6.66 (s, 2H), 4.13-4.02 (m, 1H), 3.99-3.88 (m, 2H), 3.11-3.05 (m, 1H), 3.04-2.94 (m, 1H), 2.73 (d, J=9.0 Hz, 1H), 2.37-2.26 (m, 2H), 1.94-1.85 (m, 1H), 1.71-1.56 (m, 2H), 1.30-1.23 (m, 1H), 1.20 (d, J=6.6 Hz, 6H).

Total proton count from structure: 21.

Total proton count from spectrum: 21.

### Compound 35: (S)-4-((2-(2,2-dimethylcyclopropyl)-6-isopropyl-7-oxo-[1,2,4]triazolo[1,5-a]pyrimidin-4(7H)-yl)methyl)benzenesulfonamide

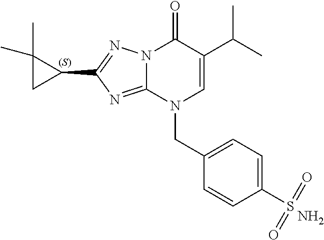

### Step 1: (S)-2,2-Dethylcyclopropane-1-carbonyl chloride

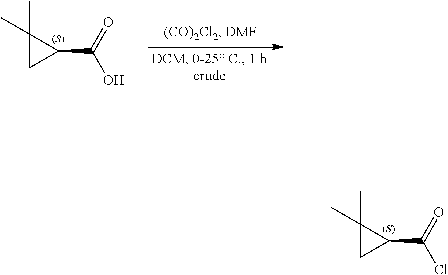

To a stirred solution of(S)-2,2-dimethylcyclopropane-1-carboxylic acid (2.00 g, 17.5 mmol, 1.0 equiv) and DMF (128 mg, 1.75 mmol, 0.10 equiv) in DCM (80 mL) was added (CO)₂Cl₂ (11.12 g, 87.6 mmol, 5.0 equiv) dropwise at 0° C. under nitrogen atmosphere. The resulting mixture was stirred for 1 h at 25° C. under nitrogen atmosphere. The resulting mixture was concentrated under reduced pressure to afford(S)-2,2-dethylcyclopropane-1-carbonyl chloride (crude). The crude products were used in the next step directly without further purification.

### Step 2: (S)-2-(2,2-Dimethylcyclopropane-1-carbonyl) hydrazine-1-carboximidamide

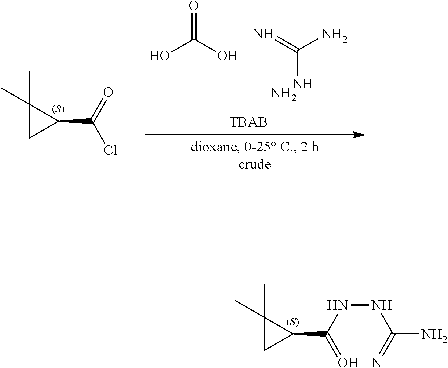

To a stirred solution of aminoguanidine; carbonic acid (4.11 g, 30.2 mmol, 2.0 equiv) and TBAB (486 mg, 1.51 mmol, 0.1 equiv) in dioxane (30 mL) was added(S)-2,2-dimethylcyclopropane-1-carbonyl chloride (2.00 g, 15.1 mmol, 1.0 equiv) dropwise at 0° C. under nitrogen atmosphere. The resulting mixture was stirred for 2 h at 25° C. under nitrogen atmosphere. The reaction was monitored by LCMS. The resulting mixture was concentrated under reduced pressure. The resulting mixture was diluted with water (20 mL). The mixture basified to pH 12 with 1 M NaOH(aq). The resulting mixture was filtered, the filter cake was washed with water (2×3 mL). The product is in the filtrate (40 ml). The filtrate was used in the next step directly without further purification.

ESI-MS m/z=171.10. [M+H]⁺; Calculated MW: 170.1.

### Step 3: (S)-3-(2,2-Dimethylcyclopropyl)-1H-1,2,4-triazol-5-amine

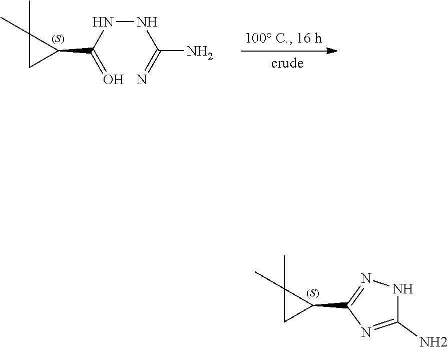

The above mixture (40 mL) was basified to pH 12 with 1M NaOH(aq.). The mixture was stirred for 4 h at 100° C. under nitrogen atmosphere. The reaction was monitored by LCMS. The mixture was allowed to cool down to 0° C. The mixture was neutralised to pH 7 with 1M HCl (aq.). The resulting mixture was extracted with EtOAc (3×40 mL). The combined organic layers were washed with brine (1×50 mL), and dried over anhydrous Na₂SO₄. After filtration, the filtrate was concentrated under reduced pressure. This resulted in(S)-3-(2,2-dimethylcyclopropyl)-1H-1,2,4-triazol-5-amine (crude).

ESI-MS m/z=153.10. [M+H]⁺; Calculated MW: 152.1.

### Step 4: (S)-2-(2,2-Dimethylcyclopropyl)-6-isopropyl-[1,2,4]triazolo[1,5-a]pyrimidin-7(4H)-one

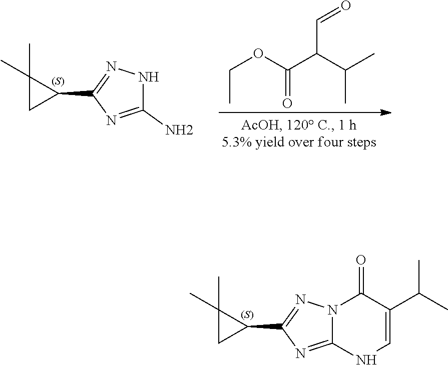

A solution of 5-[(S)-2,2-dimethylcyclopropyl]-2H-1,2,4-triazol-3-amine (1.50 g, 9.86 mmol, 1.0 equiv) and ethyl 2-formyl-3-methylbutanoate (1.56 g, 9.86 mmol, 1.0 equiv) in AcOH (30 mL) was stirred for 1 h at 120° C. under nitrogen atmosphere. The reaction was monitored by LCMS. The mixture was allowed to cool down to 25° C. The resulting mixture was concentrated under reduced pressure. The residue was purified by reverse phase flash with the following conditions (Column: C18; Mobile Phase A: Water (0.1% FA), Mobile Phase B: ACN; Gradient: 0% B to 50% B in 20 min) to afford(S)-2-(2,2-dimethylcyclopropyl)-6-isopropyl-[1,2,4]triazolo[1,5-a]pyrimidin-7(4H)-one.

ESI-MS m/z=247.20. [M+H]⁺; Calculated MW: 246.1.

¹H NMR (400 MHZ, DMSO-d₆) δ 12.92 (br, 1H), 7.69 (s, 1H), 3.03-2.90 (m, 1H), 1.96-1.83 (m, 1H), 1.23-1.14 (m, 9H), 1.11-1.09 (m, 1H), 1.04 (s, 3H), 0.98-0.92 (m, 1H).

### Step 5: (S)-4-((2-(2,2-Dimethylcyclopropyl)-6-isopropyl-7-oxo-[1,2,4]triazolo[1,5-a]pyrimidin-4(7H)-yl)methyl)benzenesulfonamide

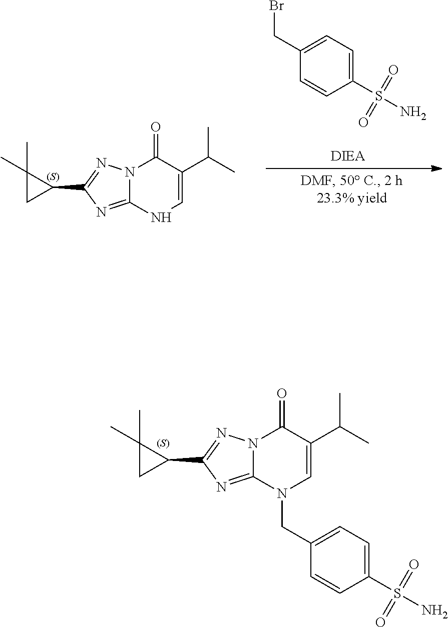

To a stirred solution of(S)-2-(2,2-dimethylcyclopropyl)-6-isopropyl-[1,2,4]triazolo[1,5-a]pyrimidin-7(4H)-one (130 mg, 0.53 mmol, 1.0 equiv) and DIEA (205 mg, 1.58 mmol, 3.0 equiv) in DMF (4 mL) was added 4-(bromomethyl)benzenesulfonamide (264 mg, 1.06 mmol, 2.0 equiv) at 25° C. under nitrogen atmosphere. The resulting mixture was stirred for 2 h at 50° C. under nitrogen atmosphere. The reaction was monitored by LCMS. The resulting mixture was concentrated under reduced pressure. The crude product was purified by Prep-HPLC with the following conditions (Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min; Gradient: 25% B to 40% B in 10 min; Wave Length: 254 nm/220 nm; RT1 (min): 9.5), the pure fractions were concentrated and then lyophilized to afford(S)-4-((2-(2,2-dimethylcyclopropyl)-6-isopropyl-7-oxo-[1,2,4]triazolo[1,5-a]pyrimidin-4(7H)-yl)methyl)benzenesulfonamide.

ESI-MS m/z=416.15. [M+H]⁺; Calculated MW: 415.2.

¹H NMR (400 MHZ, DMSO-d₆) δ 8.04 (s, 1H), 7.80-7.73 (m, 2H), 7.56-7.48 (m, 2H), 7.35 (br, 2H), 5.53-5.32 (m, 2H), 3.07-2.89 (m, 1H), 1.94-1.85 (m, 1H), 1.24-1.13 (m, 9H), 1.13-1.04 (m, 1H), 1.00 (s, 3H), 0.97-0.89 (m, 1H).

Total proton count from structure: 25.

Total proton count from spectrum: 25.

### Compound 36: rac-(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-sulfonamide

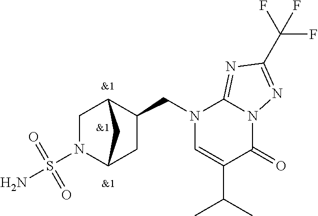 [SMILES: CC(C)c1cn(C[C@H]2C[C@H]3C[C@@H]2CN3S(N)(=O)=O)c2nc(C(F)(F)F)nn2c1=O]

### Step 1: 6-Isopropyl-2-(trifluoromethyl)-[1,2,4]triazolo[1,5-a]pyrimidin-7(4H)-one

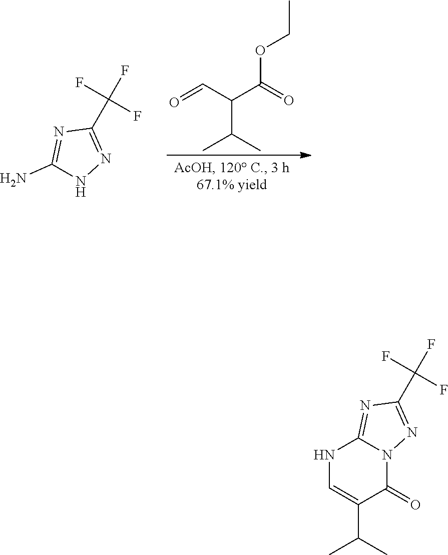

To a stirred solution of 5-(trifluoromethyl)-2H-1,2,4-triazol-3-amine (4.00 g, 26.3 mmol, 1.5 equiv) in AcOH (40 mL) were added ethyl 2-formyl-3-methylbutanoate (3.00 g, 18.1 mmol, 1.0 equiv) at 25° C. The resulting mixture was stirred for an additional 3 h at 120° C. under nitrogen atmosphere. The desired product could be detected by LCMS. The resulting mixture was diluted with water (50 mL). The resulting mixture was extracted with CH₂Cl₂ (3×50 mL). The combined organic layers were washed with brine (1×50 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (0.1% FA), 50% to 70% gradient in 20 min; detector: UV 254 nm. This resulted in 6-isopropyl-2-(trifluoromethyl)-4H-[1,2,4]triazolo[1,5-a]pyrimidin-7-one.

ESI-MS m/z=247.1 [M+H]⁺; Calculated: 246.1.

### Step 2: Rac-tert-butyl(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-carboxylate

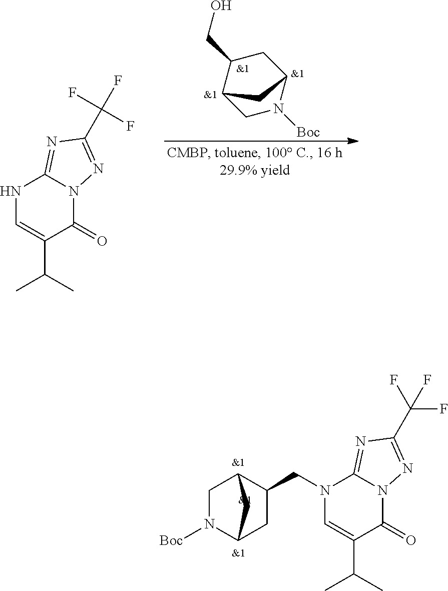 [SMILES: CC(C)c1c[nH]c2nc(C(F)(F)F)nn2c1=O ; CC(C)(C)OC(=O)N1C[C@H]2C[C@@H]1C[C@@H]2CO ; CC(C)c1cn(C[C@H]2C[C@H]3C[C@@H]2CN3C(=O)OC(C)(C)C)c2nc(C(F)(F)F)nn2c1=O]

To a stirred solution of 6-isopropyl-2-(trifluoromethyl)-4H-[1,2,4]triazolo[1,5-a]pyrimidin-7-one (542 mg, 2.20 mmol, 1.0 equiv) and tert-butyl(1R,4S,5S)-5-(hydroxymethyl)-2-azabicyclo[2.2.1]heptane-2-carboxylate (500 mg, 2.20 mmol, 1.0 equiv) in Toluene (20 mL) were added CMBP (5.31 g, 22.0 mmol, 10.0 equiv) at 25° C. under nitrogen atmosphere. The resulting mixture was stirred for 16 h at 100° C. under nitrogen atmosphere. The desired product could be detected by LCMS. The mixture was allowed to cool down to 25° C. The resulting mixture was diluted with water (100 mL). The resulting mixture was extracted with EtOAc (3×100 mL). The combined organic layers were washed with brine (100 mL), and dried over anhydrous Na₂SO₄. After filtration, the filtrate was concentrated under reduced pressure. The residue was purified by silica gel column chromatography, eluted with PE/EA (1:1) to afford rac-tert-butyl(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-carboxylate. The residue was purified by reversed-phase flash chromatography with the following conditions: column: C18; mobile phase: MeCN in Water (0.1% FA), 30% to 50% gradient in 20 min; detector: UV 254 nm. This resulted in rac-tert-butyl(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-carboxylate.

ESI-MS m/z=456.2 [M+H]⁺; Calculated: 455.2.

¹H NMR (300 MHZ, DMSO-d₆) δ 8.10 (s, 1H), 4.10-3.96 (m, 2H), 3.18 (s, 1H), 3.17-2.96 (m, 2H), 2.82 (d, J=9.6 Hz, 1H), 2.39-2.26 (m, 2H), 1.73 (d, J=10.4 Hz, 2H), 1.53 (t, J=11.7 Hz, 1H), 1.36 (d, J=5.6 Hz, 10H), 1.23 (d, J=6.9 Hz, 6H).

### Step 3: Rac-4-(((1R,4S,5S)-2-azabicyclo[2.2.1]heptan-5-yl)methyl)-6-isopropyl-2-(trifluoromethyl)-[1,2,4]triazolo[1,5-a]pyrimidin-7(4H)-one hydrochloride

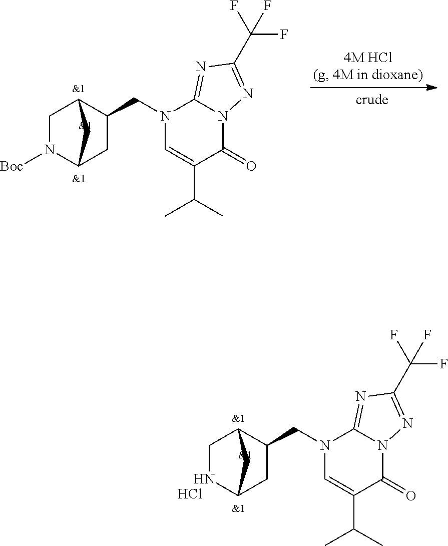 [SMILES: CC(C)c1cn(C[C@H]2C[C@H]3C[C@@H]2CN3C(=O)OC(C)(C)C)c2nc(C(F)(F)F)nn2c1=O ; Cl ; CC(C)c1cn(C[C@H]2C[C@H]3C[C@@H]2CN3)c2nc(C(F)(F)F)nn2c1=O]

A solution of rac-tert-butyl(1R,4R,5R)-5· ((6-isopropyl-7-oxo-2· (trifluoromethyl)-[1,2,4]triazolo[1,5-a]pyrimidin-4(7H)-yl)methyl)-2-azabicyclo (200 mg, 0.45 mmol, 1.0 equiv) in HCl (g, 4 M in 1,4-dioxane, 3 mL) was stirred for 1 h at 25° C. under argon atmosphere. The desired product could be detected by LCMS. The resulting mixture was concentrated under reduced pressure. The residue was purified by trituration with diethyl ether (5 mL). This resulted in rac-4-(((1R,4S,5S)-2-azabicyclo[2.2.1]heptan-5-yl)methyl)-6-isopropyl-2-(trifluoromethyl)-[1,2,4]triazolo[1,5-a]pyrimidin-7(4H)-one hydrochloride (crude).

ESI-MS m/z=356.1 [M−HCl+H]⁺; Calculated: 391.1.

### Step 4: Rac-(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-sulfonamide

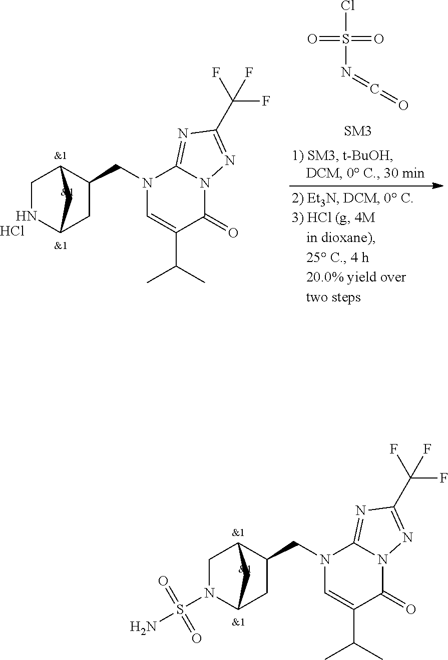 [SMILES: Cl ; CC(C)c1cn(C[C@H]2C[C@H]3C[C@@H]2CN3)c2nc(C(F)(F)F)nn2c1=O ; O=C=NS(=O)(=O)Cl ; CC(C)c1cn(C[C@H]2C[C@H]3C[C@@H]2CN3S(N)(=O)=O)c2nc(C(F)(F)F)nn2c1=O]

To a stirred solution of chlorosulfonyl isocyanate (891 mg, 6.30 mmol, 10.0 equiv) in DCM (5 mL) were added t-BuOH (467 mg, 6.30 mmol, 10.0 equiv) dropwise at 0° C. under nitrogen atmosphere. The resulting mixture was stirred for 0.5 h at 0° C. under nitrogen atmosphere. The above solution (2 ml) and Et₃N (637 mg, 6.30 mmol, 10.0 equiv) was added dropwise a solution of rac-4-(((1R,4S,5S)-2-azabicyclo[2.2.1]heptan-5-yl)methyl)-6-isopropyl-2-(trifluoromethyl)-[1,2,4]triazolo[1,5-a]pyrimidin-7(4H)-one hydrochloride (200 mg, 0.563 mmol, 1.0 equiv) in DCM (2 mL) at 0° C. under nitrogen atmosphere. The resulting mixture was stirred for 2 h at room temperature under nitrogen atmosphere. The reaction was monitored by LCMS. To the above mixture was added HCl (g, 4 M in 1,4-dioxane, 3 mL) at 25° C. The resulting mixture was stirred 4 h at 25° C. The reaction was monitored by LCMS. The resulting mixture was concentrated under reduced pressure. The residue was dissolved in water (20 mL). The resulting mixture was extracted with EtOAc (3×50 mL). The combined organic layers were washed with brine (1×10 mL), and dried over anhydrous Na₂SO₄. After filtration, the filtrate was concentrated under reduced pressure. The crude product (150 mg) was purified by Prep-HPLC with the following conditions (Column: XBridge Prep phenly Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 22% B to 42% B in 10 min; Wave Length: 254 nm/220 nm nm; RT1 (min): 9.86) the pure fraction was concentrated then lyophilized to afford rac-(1R,4R,5R)-5-((6-isopropyl-7-oxo-2-(trifluoromethyl)-[1,2,4]triazolo[1,5-a]pyrimidin-4(7H)-yl)methyl)-2-azabicyclo[2.2.1]heptane-2-sulfonamide.

ESI-MS m/z=435.13 [M+H]⁺; Calculated: 434.1.

¹H NMR (400 MHZ, DMSO-d₆) δ 8.10 (s, 1H), 6.67 (s, 2H), 4.07 (m, 2H), 3.94 (s, 1H), 3.12-2.97 (m, 2H), 2.71 (d, J=9.0 Hz, 1H), 2.34 (dd, J=11.6, 4.5 Hz, 2H), 1.89 (m, 1H), 1.72-1.57 (m, 2H), 1.32-1.17 (m, 7H).

Total proton count from structure: 21

Total proton count from spectrum: 21

### Compound 37: (1R,4R,5R)-5-((6-isopropyl-7-oxo-[1,2,4]triazolo[1,5-a]pyrimidin-4(7H)-yl)methyl)-2-azabicyclo[2.2.1]heptane-2-sulfonamide

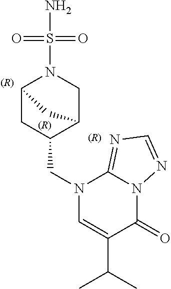

### Step 1: (1S,4R,5R)-2-(tert-butoxycarbonyl)-2-azabicyclo[2.2.1]heptane-5-carboxylic acid

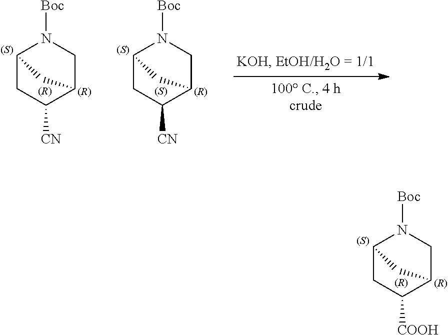

A solution of tert-butyl(1S,4R,5R)-5-cyano-2-azabicyclo[2.2.1]heptane-2-carboxylatetert-butyl (350 mg, 1.57 mmol, 0.5 equiv), (1S,4R,5S)-5-cyano-2-azabicyclo[2.2.1]heptane-2-carboxylate (350 mg, 1.57 mmol, 0.5 equiv) and KOH (883 mg, 15.7 mmol, 5.0 equiv) in EtOH (4 mL) and H₂O (4 mL) was stirred for 4 h at 100° C. The mixture was allowed to cool down to 25° C. The reaction was monitored by LCMS. The desired product could be detected by LCMS. The resulting mixture was washed with water (100 mL) The mixture was acidified to pH 6 with conc. HCl. The resulting mixture was extracted with EtOAc (3×100 mL). The combined organic layers were washed with water (1×50 mL), and dried over anhydrous Na₂SO₄. After filtration, the filtrate was concentrated under reduced pressure. The crude product was used in the next step directly without further purification.

ESI-MS m/z=186.0 [M-56+H]⁺; Calculated: 241.2.

### Step 2: Tert-butyl(1S,4R,5R)-5-(hydroxymethyl)-2-azabicyclo[2.2.1]heptane-2-carboxylate

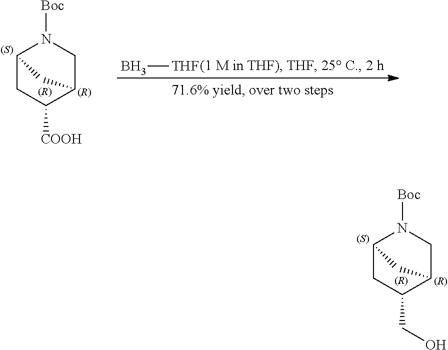

To a stirred solution of (1S,4R,5R)-2-(tert-butoxycarbonyl)-2-azabicyclo[2.2.1]heptane-5-carboxylic acid (800 mg, 3.31 mmol, 1.0 equiv) in THF (15 mL) was added BH₃-THF (8.3 mL, 8.29 mmol, 2.5 equiv, 1 M in THF) dropwise at 0° C. under nitrogen atmosphere. The resulting mixture was stirred for 2 h at 25° C. The reaction was monitored by LCMS. The reaction was quenched by the addition of water (20 mL) at 0° C. The resulting mixture was diluted with EtOAc (300 mL), The resulting mixture was washed with water (3×80 mL). The combined organic layers were washed with brine (1×80 mL), and dried over anhydrous Na₂SO₄. After filtration, the filtrate was concentrated under reduced pressure. The residue was purified by reversed-phase flash chromatography with the following conditions: column: C18; mobile phase: MeCN in Water (0.1% FA), 10% to 30% gradient in 15 min; detector: UV 254 nm. This resulted in tert-butyl(1S,4R,5R)-5-(hydroxymethyl)-2-azabicyclo[2.2.1]heptane-2-carboxylate.

ESI-MS m/z=172.1 [M-56+H]⁺; Calculated: 227.2.

¹H NMR (400 MHZ, DMSO-d₆) δ 4.65-3.57 (m, 1H), 3.99 (d, J=15.3 Hz, 1H), 3.25-3.09 (m, 3H), 2.84 (d, J=16 Hz, 1H), 2.54-2.50 (m, 1H), 2.44-2.40 (m, 1H), 1.70 (d, J=6.8 Hz, 1H), 1.66-1.54 (m, 1H), 1.47-1.42 (m, 1H), 1.40-1.34 (m, 9H), 1.13-1.01 (m, 1H).

### Step 3: 6-Isopropyl-4H-[1,2,4]triazolo[1,5-a]pyrimidin-7-one

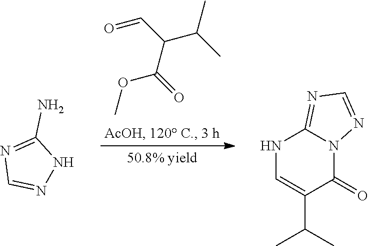

To a stirred solution of 5-amino-1H-1,2,4-triazole (642 mg, 7.63 mmol, 1.1 equiv) in AcOH (10 mL) were added methyl 2-formyl-3-methylbutanoate (1.00 g, 6.93 mmol, 1.0 equiv) at 25° C. under nitrogen atmosphere. The resulting mixture was stirred for 3 h at 120° C. The mixture was allowed to cool down to 25° C. The reaction was monitored by LCMS. The product was precipitated by the addition of water (50 mL). The precipitated solids were collected by filtration and washed with water (3×6 mL). The solids was dried under reduced pressure to afford 6-isopropyl-4H-[1,2,4]triazolo[1,5-a]pyrimidin-7-one.

ESI-MS m/z=179.1 [M+H]⁺; Calculated: 178.2.

¹H NMR (400 MHZ, DMSO-d₆) δ 13.15 (s, 1H), 8.21 (s, 1H), 7.80 (d, J=0.8 Hz, 1H), 3.08-2.93 (m, 1H), 1.21 (s, 3H), 1.19 (s, 3H).

### Step 4: Tert-butyl(1R,4R,5R)-5-((6-isopropyl-7-oxo-[1,2,4]triazolo[1,5-a]pyrimidin-4(7H)-yl)methyl)-2-azabicyclo[2.2.1]heptane-2-carboxylate

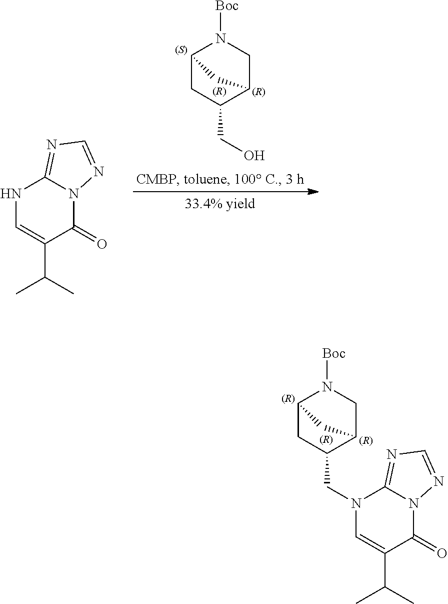

To a stirred solution of 6-isopropyl-4H-[1,2,4]triazolo[1,5-a]pyrimidin-7-one (250 mg, 1.40 mmol, 1.0 equiv) and tert-butyl(1S,4R,5R)-5-(hydroxymethyl)-2-azabicyclo[2.2.1]heptane-2-carboxylate (415 mg, 1.82 mmol, 1.3 equiv) in toluene (3 mL) were added CMBP (3.33 g, 14.0 mmol, 10.0 equiv) at 25° C. under nitrogen atmosphere. The resulting mixture was stirred for 3 h at 100° C. The mixture was allowed to cool down to 25° C. The reaction was monitored by LCMS. The residue was purified by silica gel column chromatography, eluted with PE/EA (1:2) to afford tert-butyl(1R,4R,5R)-5-((6-isopropyl-7-oxo-[1,2,4]triazolo[1,5-a]pyrimidin-4(7H)-yl)methyl)-2-azabicyclo[2.2.1]heptane-2-carboxylate.

ESI-MS m/z=388.2. [M+H]⁺; Calculated: 387.2

### Step 5: 4-(((1S,4R,5R)-2-azabicyclo[2.2.1]heptan-5-yl)methyl)-6-isopropyl-[1,2,4]triazolo[1,5-a]pyrimidin-7(4H)-one hydrochloride

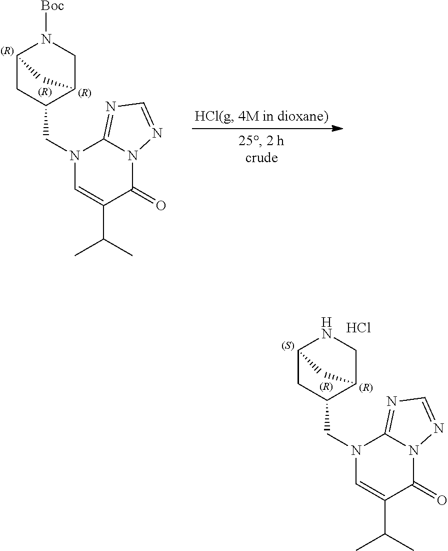

To a stirred solution of tert-butyl(1R,4R,5R)-5-((6-isopropyl-7-oxo-[1,2,4]triazolo[1,5-a]pyrimidin-4(7H)-yl)methyl)-2-azabicyclo[2.2.1]heptane-2-carboxylate (300 mg, 0.77 mmol, 1.0 equiv) was added HCl (g, 4 M in 1,4-dioxane, 6 mL) at 25° C. under nitrogen atmosphere. The resulting mixture was stirred for 2 h at 25° C. The reaction was monitored by LCMS. The resulting mixture was concentrated under reduced pressure. The crude product was used in the next step directly without further purification.

ESI-MS m/z=288.1 [M−HCl+H]⁺; Calculated: 323.3.

### Step 6: (1R,4R,5R)-5-((6-isopropyl-7-oxo-[1,2,4]triazolo[1,5-a]pyrimidin-4(7H)-yl)methyl)-2-azabicyclo[2.2.1]heptane-2-sulfonamide

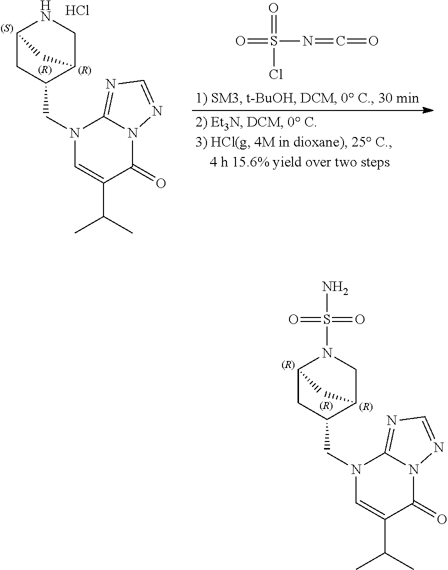

To a stirred solution of chlorosulfonyl isocyanate (344 mg, 2.43 mmol, 2.0 equiv) in DCM (5 mL) was added t-BuOH (135 mg, 1.82 mmol, 1.5 equiv) at 0° C. under nitrogen atmosphere. A solution of 4-(((1S,4R,5R)-2-azabicyclo[2.2.1]heptan-5-yl)methyl)-6-isopropyl-[1,2,4]triazolo[1,5-a]pyrimidin-7(4H)-one hydrochloride (350 mg, 1.21 mmol, 1.0 equiv) in DCM (5 mL) was treated with Et₃N (369 mg, 3.65 mmol, 3.0 equiv) for 5 min at room temperature under nitrogen atmosphere followed by the addition of above mixture dropwise at 0° C. The resulting mixture was stirred for 2 h at 25° C. under nitrogen atmosphere. The reaction was monitored by LCMS. To the above mixture was added HCl (g, 4 M in 1,4-dioxane, 5 mL) at room temperature. The resulting mixture was stirred 5 h at room temperature. The reaction was monitored by LCMS. The resulting mixture was concentrated under reduced pressure. The residue was dissolved in water (20 mL). The resulting mixture was extracted with EtOAc (2×50 mL). The combined organic layers were washed with brine (1×20 mL), and dried over anhydrous Na₂SO₄. After filtration, the filtrate was concentrated under reduced pressure. The residue was purified by reversed-phase flash chromatography with the following conditions: Column: Xbridge Phenyl OBD Column, 19*150 mm, 5 m; Mobile Phase A: Water (10 mmol/L NH₄HCO₃+0.05% NH₃H₂O, Mobile Phase B: ACN; Flow rate: 60 mL/min mL/min; Gradient: 13% B to 23% B in 10 min; Wave Length: 254 nm/220 nm nm; RT1 (min)) the pure fraction was concentrated then lyophilized to afford (1R,4R,5R)-5-((6-isopropyl-7-oxo-[1,2,4]triazolo[1,5-a]pyrimidin-4(7H)-yl)methyl)-2-azabicyclo[2.2.1]heptane-2-sulfonamide.

ESI-MS m/z=367.10 [M+H]⁺; Calculated: 366.1.

¹H NMR (400 MHZ, DMSO-d₆) δ 8.24 (s, 1H), 8.01 (s, 1H), 6.67 (s, 2H), 4.14 (dd, J=13.8, 9.0 Hz, 1H), 4.02-3.91 (m, 2H), 3.07 (dd, J=9.1, 3.5 Hz, 1H), 3.05-2.95 (m, 1H), 2.71 (d, J=9.1 Hz, 1H), 2.41-2.29 (m, 2H), 1.93-1.84 (m, 1H), 1.68 (d, J=10.2 Hz, 1H), 1.61 (d, J=10.4 Hz, 1H), 1.32-1.25 (m, 1H), 1.22 (s, 3H), 1.21 (s, 3H).

Total proton counts from structure: 22.

Total proton counts from spectrum: 22.

### Compound 38: 4-((2-cyclopropyl-6-methyl-7-oxo-[1,2,4]triazolo[1,5-a]pyrimidin-4(7H)-yl)methyl)benzenesulfonamide

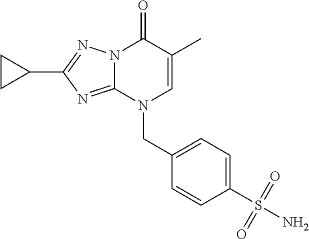 [SMILES: Cc1cn(Cc2ccc(S(N)(=O)=O)cc2)c2nc(C3CC3)nn2c1=O]

### Step 1: 2-Cyclopropyl-6-methyl-4H-[1,2,4]triazolo[1,5-a]pyrimidin-7-one

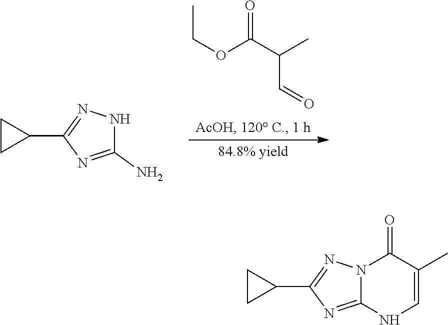

A mixture of ethyl 2-methyl-3-oxopropanoate (500 mg, 3.84 mmole, 1.0 equiv) and 5-cyclopropyl-2H-1,2,4-triazol-3-amine (620 mg, 4.99 mmole, 1.3 equiv) in AcOH (5 ml) was stirred for 1 h at 120° C. The mixture was allowed to cool down to 25° C. The reaction progress was monitored by LCMS. The product was precipitated by the addition of water. The precipitated solids were collected by filtration and washed with water (2×10 mL). This resulted in 2-cyclopropyl-6-methyl-4H-[1,2,4]triazolo[1,5-a]pyrimidin-7-one.

ESI-MS m/z=191.1. [M+H]⁺; Calculated: 190.1.

### Step 2: 4-((2-Cyclopropyl-6-methyl-7-oxo-[1,2,4]triazolo[1,5-a]pyrimidin-4(7H)-yl)methyl)benzenesulfonamide

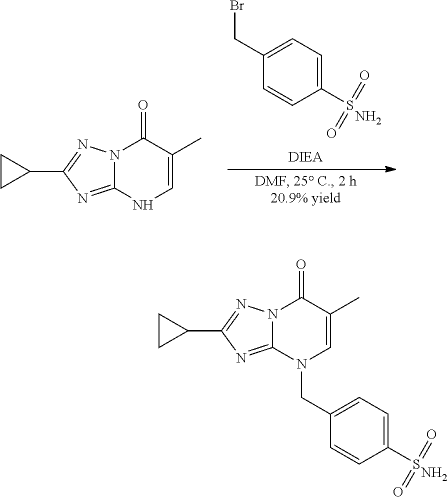

A solution of 2-cyclopropyl-6-methyl-4H-[1,2,4]triazolo[1,5-a]pyrimidin-7-one (300 mg, 1.57 mmol, 1.0 equiv) and 4-(bromomethyl)benzenesulfonamide (473 mg, 1.89 mmol, 1.2 equiv) and DIEA (612 mg, 4.73 mmol, 3.0 equiv) in DMF (3 mL) was stirred for 2 h at 25° C. under nitrogen atmosphere. The reaction progress was monitored by LCMS. The resulting mixture was diluted with EtOAc (15 mL). The resulting mixture was washed with 5×15 ml of water. The organic layer was concentrated under reduced pressure to afford crude product. The crude product was purified by Prep-HPLC with the following conditions: (Water (0.1% FA), Mobile Phase B: ACN; Flow rate: 60 mL/min; Gradient: 1% B to 14% B in 10 min; Wave Length: 254 nm/220 nm nm; RT1 (min): 9.27) the pure fraction was concentrated then lyophilized to afford 4-((2-cyclopropyl-6-methyl-7-oxo-[1,2,4]triazolo[1,5-a]pyrimidin-4(7H)-yl)methyl)benzenesulfonamide.

ESI-MS m/z=360.15 [M+H]⁺; Calculated: 359.1.

¹H NMR (300 MHz, DMSO-d₆) δ 8.09 (d, J=1.2 Hz, 1H), 7.84-7.76 (m, 2H), 7.58-7.50 (m, 2H), 7.37 (s, 2H), 5.35 (s, 2H), 2.12-1.96 (m, 4H), 1.04-1.00 (m, 2H), 0.97-0.85 (m, 2H).

Total proton count from structure: 17.

Total proton count from spectrum: 17.

### Compound 39: (1S,4S,5S)-5-((3-Isopropyl-4-oxo-7,8,9,10-tetrahydropyrimido[1,2-b]indazol-1 (4H)-yl)methyl)-2-azabicyclo[2.2.1]heptane-2-sulfonamide

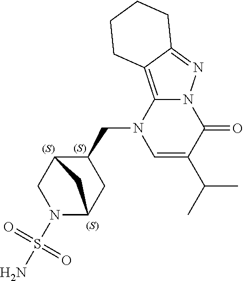

### Step 1: 4,5,6,7-Tetrahydro-2H-indazol-3-amine

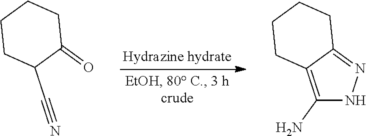 [SMILES: N#CC1CCCCC1=O ; Nc1[nH]nc2c1CCCC2]

To a stirred solution of 2-oxocyclohexane-1-carbonitrile (2.00 g, 16.2 mmol, 1.0 equiv) in EtOH (20.00 mL) was added hydrazine hydrate (98% w.t. in H₂O) (1.63 g, 32.5 mmol, 2.0 equiv) at 25° C. under nitrogen atmosphere. The resulting mixture was stirred for 3 h at 80° C. under nitrogen atmosphere. The resulting mixture was concentrated under reduced pressure. This resulted in 4,5,6,7-tetrahydro-2H-indazol-3-amine (crude).

ESI-MS m/z=138.0 [M+H]⁺; Calculated: 137.1

### Step 2: 3-Isopropyl-7,8,9,10-tetrahydropyrimido[1,2-b]indazol-4 (1H)-one

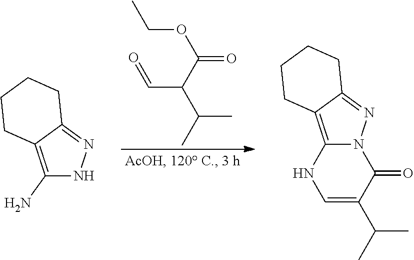 [SMILES: Nc1[nH]nc2c1CCCC2 ; CCOC(=O)C(C=O)C(C)C ; CC(C)c1c[nH]c2c3c(nn2c1=O)CCCC3]

To a stirred solution of 4,5,6,7-tetrahydro-2H-indazol-3-amine (2.00 g, 14.6 mmol, 1.0 equiv) in AcOH (15.00 mL) was added ethyl 2-formyl-3-methylbutanoate (2.77 g, 17.5 mmol, 1.2 equiv) at 25° C. under nitrogen atmosphere. The resulting mixture was stirred for 3 h at 120° C. under nitrogen atmosphere. The reaction was monitored by LCMS. The mixture was allowed to cool down to 25° C. The product was precipitated by the addition of water. The precipitated solids were collected by filtration and washed with water (3×10 mL), to afford 3-isopropyl-7,8,9,10-tetrahydropyrimido[1,2-b]indazol-4 (1H)-one.

ESI-MS m/z=232.0 [M+H]⁺; Calculated: 231.1.

¹H NMR (400 MHZ, DMSO-d₆) δ 11.96 (s, 1H), 7.54 (s, 1H), 2.99 (p, J=6.9 Hz, 1H), 2.64 (t, J=6.0 Hz, 2H), 2.54 (s, 2H), 1.80-1.70 (m, 4H), 1.17 (d, J=6.9 Hz, 6H).

### Step 3: Tert-butyl(1S,4S,5S)-5-((3-isopropyl-4-oxo-7,8,9,10-tetrahydropyrimido[1,2-b]indazol-1 (4H)-yl)methyl)-2-azabicyclo[2.2.1]heptane-2-carboxylate

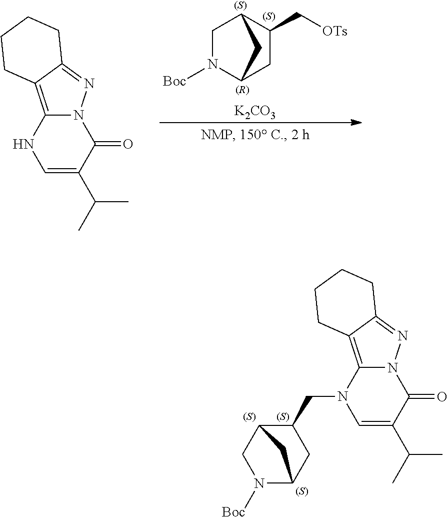

To a stirred solution of tert-butyl(1R,4S,5S)-5-{[(4-methylbenzenesulfonyl)oxy]methyl}-2-azabicyclo[2.2.1]heptane-2-carboxylate (200 mg, 0.52 mmol, 1.0 equiv) and 3-isopropyl-1H,7H,8H,9H,10H-pyrimido[1,2-b]indazol-4-one (146 mg, 0.63 mmol, 1.2 equiv) in NMP (3.00 mL) was added K₂CO₃ (217 mg, 1.57 mmol, 3.0 equiv) at 25° C. under nitrogen atmosphere. The resulting mixture was stirred for 2 h at 150° C. under nitrogen atmosphere. The mixture was allowed to cool down to 25° C. The residue was purified by reversed-phase flash chromatography with the following conditions: column, C18; mobile phase, MeCN in water (0.1% FA), 10% to 60% gradient in 40 min; detector, UV 254 nm, to afford tert-butyl(1S,4S,5S)-5-((3-isopropyl-4-oxo-7,8,9,10-tetrahydropyrimido[1,2-b]indazol-1 (4H)-yl)methyl)-2-azabicyclo[2.2.1]heptane-2-carboxylate.

ESI-MS m/z=441.3 [M+H]⁺; Calculated: 440.3.

¹H NMR (400 MHZ, DMSO-d₆) δ 7.68 (s, 1H), 4.14-4.03 (m, 2H), 3.95 (dd, J=14.5, 6.8 Hz, 1H), 3.13 (ddd, J=13.7, 9.4, 3.3 Hz, 1H), 2.99 (p, J=6.9 Hz, 1H), 2.85 (d, J=9.6 Hz, 1H), 2.79 (d, J=6.0 Hz, 2H), 2.67 (q, J=3.8, 2.6 Hz, 2H), 2.33-2.25 (m, 1H), 2.08 (d, J=8.1 Hz, 1H), 1.82-1.63 (m, 6H), 1.53 (dd, J=17.6, 10.1 Hz, 1H), 1.35 (d, J=8.5 Hz, 10H), 1.17 (d, J=6.9 Hz, 6H).

### Step 4: 1-(((1R,4S,5S)-2-Azabicyclo[2.2.1]heptan-5-yl)methyl)-3-isopropyl-7,8,9,10-tetrahydropyrimido[1,2-b]indazol-4 (1H)-one HCl salt

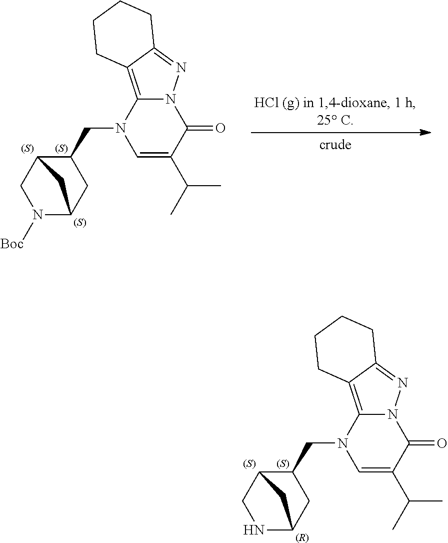

To a stirred solution of tert-butyl(1S,4S,5S)-5-({3-isopropyl-4-oxo-7H,8H,9H, 10H-pyrimido[1,2-b]indazol-1-yl}methyl)-2-azabicyclo[2.2.1]heptane-2-carboxylate (150 mg, 0.34 mmol, 1.0 equiv) in HCl (gas, 4 M in 1,4-dioxane, 5.00 mL) at 25° C. under nitrogen atmosphere. The resulting mixture was stirred for 1 h at 25° C. under nitrogen atmosphere. The reaction was monitored by LCMS. The residue was purified by trituration with ethyl ether (5.00 mL) to afford 1-(((1R,4S,5S)-2-Azabicyclo[2.2.1]heptan-5-yl)methyl)-3-isopropyl-7,8,9,10-tetrahydropyrimido[1,2-b]indazol-4 (1H)-one HCl salt.

ESI-MS m/z=341.2 [M+H]⁺; Calculated: 340.2.

### Step 5: (1S,4S,5S)-5-((3-Isopropyl-4-oxo-7,8,9,10-tetrahydropyrimido[1,2-b]indazol-1 (4H)-yl)methyl)-2-azabicyclo[2.2.1]heptane-2-sulfonamide

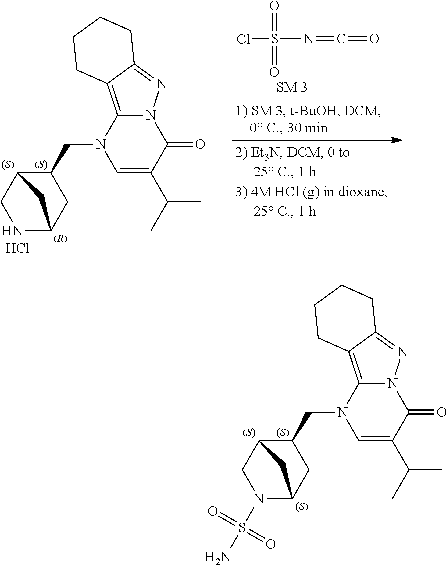

To a stirred solution of chlorosulfonyl isocyanate (424 mg, 2.10 mmol, 6.0 equiv) in DCM (3.00 mL) was added t-BuOH (333 mg, 4.49 mmol, 9.0 equiv) at 0° C. under nitrogen atmosphere. A solution of 1-[(1R,4S,5S)-2-azabicyclo[2.2.1]heptan-5-ylmethyl]-3-isopropyl-7H,8H,9H, 10H-pyrimido[1,2-b]indazol-4-one HCl salt (170 mg, 0.50 mmol, 1.0 equiv) in DCM (3.00 mL) was treated with Et₃N (505 mg, 4.99 mmol, 10.0 equiv) for 5 min at 25° C. under nitrogen atmosphere followed by the addition of the above mixture dropwise at 0° C. The resulting mixture was stirred for 1 h at 25° C. under nitrogen atmosphere. The reaction was monitored by LCMS. To the above mixture was added HCl (gas, 4 M in 1,4-dioxane, 5.00 mL) at room temperature. The resulting mixture was stirred 1 h at 25° C. The reaction was monitored by LCMS. The resulting mixture was concentrated under reduced pressure. The resulting mixture was diluted with water (30 mL). The mixture was basified to pH 8 with 1M NaOH. The resulting mixture was extracted with EtOAc (3×40 mL), and dried over anhydrous Na₂SO₄. After filtration, the filtrate was concentrated under reduced pressure. The crude product was purified by Prep-HPLC with the following conditions (Column: XBridge Prep OBD C18 Column 30*150 mm, 5 m; Mobile Phase A: Water (10 mmol/L NH₄HCO₃+0.1% NH₃·H₂O), Mobile Phase B: ACN; Flow rate: 60 mL/min mL/min; Gradient: 18% B to 38% B in 8 min; Wave Length: 254 nm/220 nm nm; RT1 (min): 8.79), the pure fraction was concentrated then lyophilized to afford (1S,4S,5S)-5-((3-Isopropyl-4-oxo-7,8,9,10-tetrahydropyrimido[1,2-b]indazol-1 (4H)-yl)methyl)-2-azabicyclo[2.2.1]heptane-2-sulfonamide.

ESI-MS m/z=420.15 [M+H]⁺; Calculated: 419.2.

¹H NMR (400 MHZ, DMSO-d₆) δ 7.69 (s, 1H), 6.67 (s, 2H), 4.08-3.92 (m, 3H), 3.09 (dd, J=9.2, 3.5 Hz, 1H), 2.99 (p, J=7.0 Hz, 1H), 2.83-2.73 (m, 3H), 2.67 (s, 2H), 2.30 (s, 1H), 2.09 (q, J=7.9, 7.3 Hz, 1H), 1.91 (t, J=10.5 Hz, 1H), 1.78-1.74 (m, 4H), 1.69 (d, J=10.3 Hz, 1H), 1.62 (d, J=10.3 Hz, 1H), 1.27 (dt, J=17.2, 5.4 Hz, 1H), 1.17 (dd, J=6.9, 2.9 Hz, 6H).

Total proton count from structure: 29.

Total proton count from spectrum: 29.

### Compound 40: 3-Isopropyl-1-(4-(S-methylsulfonimidoyl)benzyl)-1,9-dihydro-4H,7H-furo[3′,4′: 3,4]pyrazolo[1,5-a]pyrimidin-4-one

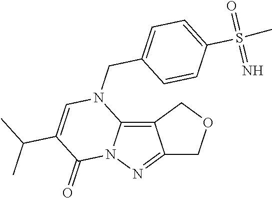 [SMILES: CC(C)c1cn(Cc2ccc(S(C)(=N)=O)cc2)c2c3c(nn2c1=O)COC3]

### Step 1: 2H,4H,6H-furo[3,4-c]pyrazol-3-amine

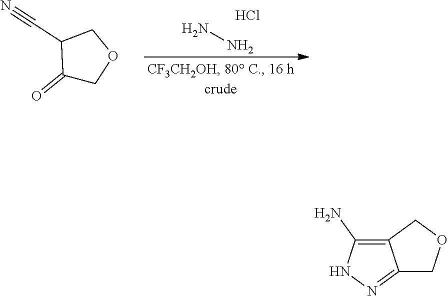

A mixture of 4-oxooxolane-3-carbonitrile (7.00 g, 63.0 mmol, 1.0 equiv) and hydrazine hydrochloride (8.63 g, 126.0 mmol, 2.0 equiv) in CF₃CH₂OH (140 mL) was stirred for 16 h at 80° C. under nitrogen atmosphere. The desired product could be detected by LCMS. The mixture was allowed to cool down to 25° C. The resulting mixture was concentrated under reduced pressure to afford 2H,4H,6H-furo[3,4-c]pyrazol-3-amine (crude).

ESI-MS m/z=125.95 [M+H]⁺; Calculated: 125.1.

### Step 2: 3-Amino-4H,6H-furo[3,4-c]pyrazole-2-carboxylate

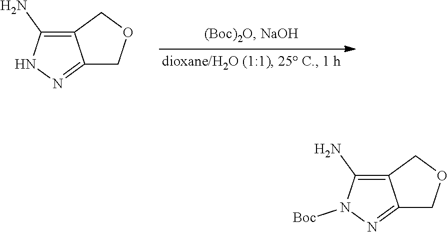 [SMILES: Nc1[nH]nc2c1COC2 ; CC(C)(C)OC(=O)n1nc2c(c1N)COC2]

To a stirred mixture of 2H,4H,6H-furo[3,4-c]pyrazol-3-amine (14.00 g, 63.6 mmol, 1.0 equiv) and (Boc) 2O (41.7 g, 191.0 mmol, 3.0 equiv) in dioxane (70 mL) was added NaOH (6.37 g, 159.2 mmol, 2.5 equiv) in H₂O (20 mL) at 25° C. under nitrogen atmosphere. The resulting mixture was stirred for 1 h at 25° C. under nitrogen atmosphere. The desired product could be detected by LCMS. The resulting mixture was diluted with water (200 mL). The resulting mixture was extracted with EA (5×200 mL). The combined organic layers were washed with brine (2×200 mL), and dried over anhydrous Na₂SO₄. After filtration, the filtrate was concentrated under reduced pressure. The residue was purified by reverse phase flash with the following conditions (column, C18; mobile phase, MeCN in Water (10 mmol/L NH₄HCO₃), 0% to 50% gradient in 25 min; detector, UV 254 nm) to afford tert-butyl 3-amino-4H,6H-furo[3,4-c]pyrazole-2-carboxylate.

ESI-MS m/z=170.10 [M-56+H]⁺; Calculated: 225.1.

¹H NMR (400 MHZ, DMSO-d₆) δ 5.61 (s, 2H), 4.83 (t, J=3.5 Hz, 2H), 4.64 (t, J=3.5 Hz, 2H), 1.48 (s, 9H).

### Step 3:10-Isopropyl-4-oxa-7,8,12-triazatricyclo[6.4.0.0\hat{ }{2,6}]dodeca-1,6,10-trien-9-one

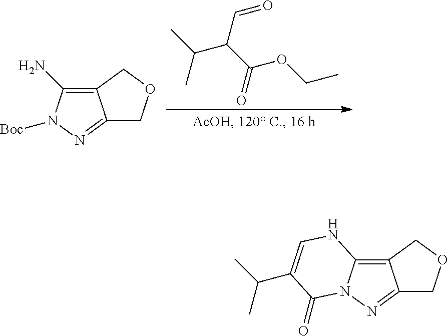

A mixture of tert-butyl 3-amino-4H,6H-furo[3,4-c]pyrazole-2-carboxylate (1.10 g, 4.88 mmol, 1.0 equiv) and ethyl 2-formyl-3-methylbutanoate (2.32 g, 14.7 mmol, 3.0 equiv) in AcOH (22 mL) was stirred for 16 h at 120° C. under nitrogen atmosphere. The desired product could be detected by LCMS. The mixture was allowed to cool down to 25° C. The precipitated solids were collected by filtration and washed with ethyl acetate (2×5 mL). The solids were diluted with ethyl acetate (50 mL) and stirred for 10 min. The precipitated solids were collected by filtration and washed with ethyl acetate (2×5 mL). The solids were concentrated under reduced pressure to afford 10-isopropyl-4-oxa-7,8,12-triazatricyclo[6.4.0.0\hat{ }{2,6}]dodeca-1,6,10-trien-9-one.

ESI-MS m/z=220.15 [M+H]⁺; Calculated: 219.1.

¹H NMR (400 MHZ, DMSO-d₆) δ 12.09 (s, 1H), 7.70 (s, 1H), 4.89-4.83 (m, 2H), 4.83-4.78 (m, 2H), 3.05-2.96 (m, 1H), 1.18 (d, J=7.0 Hz, 6H).

### Step 4: 3-Isopropyl-1-(4-(S-methylsulfonimidoyl)benzyl)-1,9-dihydro-4H,7H-furo[3′,4′: 3,4]pyrazolo[1,5-a]pyrimidin-4-one

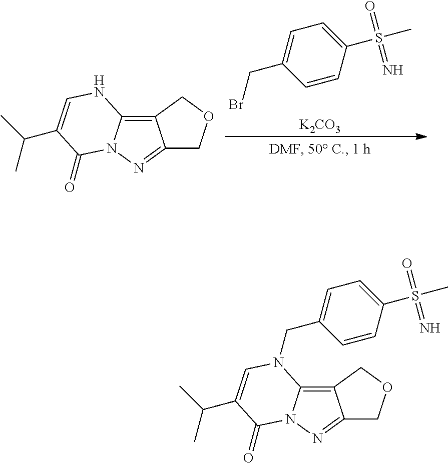

To a stirred mixture of 10-isopropyl-4-oxa-7,8,12-triazatricyclo[6.4.0.0\hat{ }{2,6}]dodeca-1,6,10-trien-9-one (150 mg, 0.68 mmol, 1.0 equiv) and K₂CO₃ (473 mg, 3.42 mmol, 5.0 equiv) in DMF (6 mL) was added [4-(bromomethyl)phenyl](imino)methyl-lambda6-sulfanone (509 mg, 2.05 mmol, 3.0 equiv) at 25° C. The resulting mixture was stirred for 1 h at 50° C. under nitrogen atmosphere. The desired product could be detected by LCMS. The resulting mixture was diluted with water (100 mL). The resulting mixture was extracted with DCM/IPA (5:1, 3×100 mL). The combined organic layers were washed with brine (1×100 mL), and dried over anhydrous Na₂SO₄. After filtration, the filtrate was concentrated under reduced pressure. The crude product (200 mg) was purified by Prep-HPLC with the following conditions (Column: XBridge Prep OBD C18 Column 30*150 mm, 5 μm; Mobile Phase A: Water (10 mmol/L NH₄HCO₃), Mobile Phase B: ACN; Flow rate: 60 mL/min; Gradient: 5% B to 30% B in 7 min; Wave Length: 254 nm/220 nm; RT1 (min): 6.5) to afford 3-isopropyl-1-(4-(S-methylsulfonimidoyl)benzyl)-1,9-dihydro-4H,7H-furo[3′,4′: 3,4]pyrazolo[1,5-a]pyrimidin-4-one.

ESI-MS m/z=387.10 [M+H]⁺; Calculated: 386.1.

¹H NMR (400 MHZ, DMSO-d₆) δ 7.99 (s, 1H), 7.92 (d, J=8.1 Hz, 2H), 7.41 (d, J=8.1 Hz, 2H), 5.38 (s, 2H), 4.75 (s, 2H), 4.56-4.45 (m, 2H), 4.25 (s, 1H), 3.10-2.98 (m, 4H), 1.23 (d, J=6.9 Hz, 6H).

Total proton count from structure: 22.

Total proton count from spectrum: 22.

### Compound 41: (1S,4S,5S)-5-((3-Isopropyl-4-oxopyrido[2′,3′: 3,4]pyrazolo[1,5-a]pyrimidin-1 (4H)-yl)methyl)-2-azabicyclo[2.2.1]heptane-2-sulfonamide

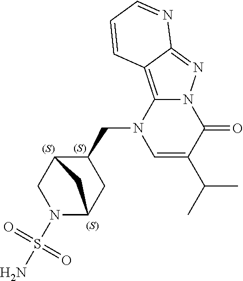

### Step 1: Tert-butyl(1S,4S,5S)-5-({2H-pyrazolo[3,4-b]pyridin-3-ylamino}methyl)-2-azabicyclo[2.2.1]heptane-2-carboxylate

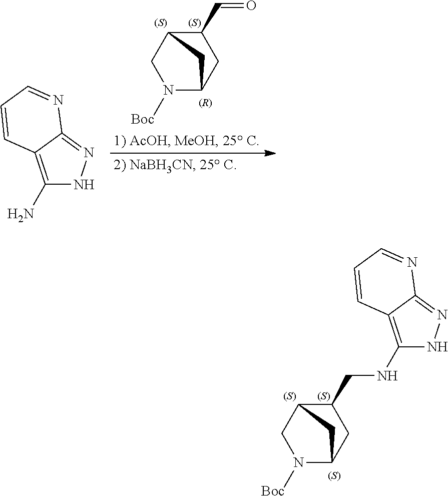

A solution of tert-butyl(1R,4S,5S)-5-formyl-2-azabicyclo[2.2.1]heptane-2-carboxylate (150 mg, 0.67 mmol, 1.0 equiv) in MeOH (2 mL) was treated with 2H-pyrazolo[3,4-b]pyridin-3-amine (178 mg, 1.33 mmol, 2.0 equiv) at 25° C. for 1 min under nitrogen atmosphere followed by the addition of AcOH (4 mg, 0.07 mmol, 0.1 equiv) at 25° C. The resulting mixture was stirred at 25° C. for 1 h under nitrogen atmosphere. To the above mixture was added NaBH₃CN (84 mg, 1.33 mmol, 2.0 equiv) in portions over 3 min at 25° C. The resulting mixture was stirred at 25° C. for an additional 1 h. The reaction was monitored by LCMS. The reaction was quenched with water at 0° C. The resulting mixture was extracted with EtOAc (3×10 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column, C18; mobile phase, MeCN in Water (0.1% FA), 20% to 30% gradient in 15 min; detector, UV 254 nm. This resulted in tert-butyl(1S,4S,5S)-5-({2H-pyrazolo[3,4-b]pyridin-3-ylamino}methyl)-2-azabicyclo[2.2.1]heptane-2-carboxylate.

ESI-MS m/z=344.1 [M+H]⁺; Calculated: 343.2.

### Step 2: 3-[(1R,4S,5S)-2-azabicyclo[2.2.1]heptan-5-ylmethyl]-5-isopropyl-3,7,8,10-tetraazatricyclo[7.4.0.0\hat{ }{2,7}]trideca-1,4,8,10,12-pentaen-6-one acetic acid salt

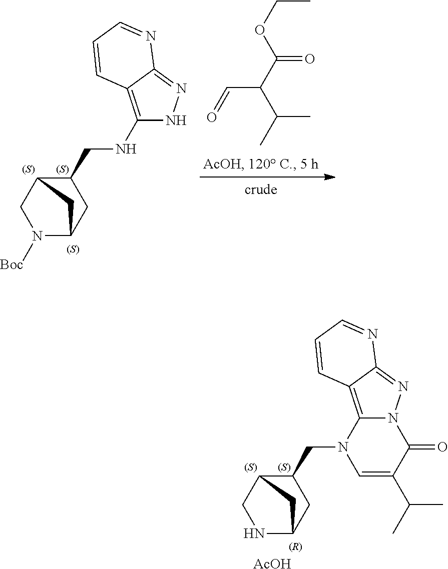

A solution of tert-butyl(1S,4S,5S)-5-({2H-pyrazolo[3,4-b]pyridin-3-ylamino}methyl)-2-azabicyclo[2.2.1]heptane-2-carboxylate (150 mg, 0.44 mmol, 1.0 equiv) and ethyl 2-formyl-3-methylbutanoate (104 mg, 0.66 mmol, 1.5 equiv) in AcOH (3 mL) was stirred at 120° C. for 5 h under nitrogen atmosphere. The reaction was monitored by LCMS. The mixture was allowed to cool down to 25° C. The resulting mixture was concentrated under reduced pressure. The residue was purified by trituration with Et₂O (10 mL). This resulted in 3-[(1R,4S,5S)-2-azabicyclo[2.2.1]heptan-5-ylmethyl]-5-isopropyl-3,7,8,10-tetraazatricyclo[7.4.0.0\hat{ }{2,7}]trideca-1,4,8,10,12-pentaen-6-one acetic acid salt (crude).

ESI-MS m/z=338.1 [M-60+H]⁺; Calculated: 397.2.

### Step 3: (1S,4S,5S)-5-((3-isopropyl-4-oxopyrido[2′,3′: 3,4]pyrazolo[1,5-a]pyrimidin-1 (4H)-yl)methyl)-2-azabicyclo[2.2.1]heptane-2-sulfonamide

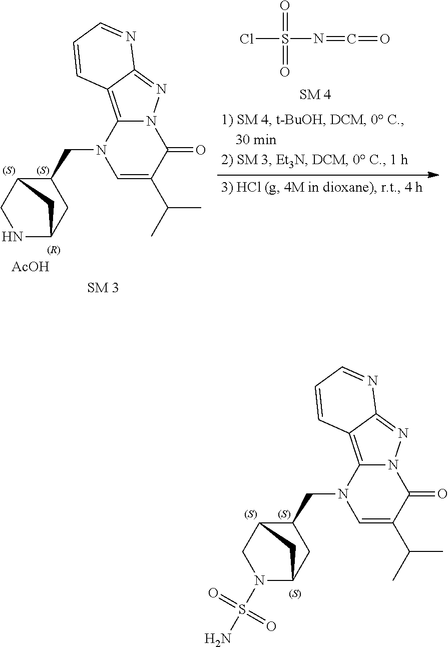

To a stirred solution of chlorosulfonyl isocyanate (341 mg, 2.41 mmol, 6.0 equiv) in DCM (2.00 mL) was added tert-butanol (267 mg, 3.61 mmol, 9.0 equiv) at 0° C. under nitrogen atmosphere. The resulting mixture was stirred for 30 min at 0° C. under nitrogen atmosphere. A solution of 3-[(1R,4S,5S)-2-azabicyclo[2.2.1]heptan-5-ylmethyl]-5-isopropyl-3,7,8,10-tetraazatricyclo[7.4.0.0\hat{ }{2,7}]trideca-1,4,8,10,12-pentaen-6-one hydrochloride (150 mg, 0.40 mmol, 1.0 equiv) in DCM (2.00 mL) was treated with Et₃N (406 mg. 4.01 mmol, 10.0 equiv) for 5 min at room temperature under nitrogen atmosphere followed by the addition of the above mixture dropwise at 0° C. The resulting mixture was stirred for 1 h at room temperature under nitrogen atmosphere. The reaction was monitored by LCMS. To the above mixture was added HCl (gas, 4 M in 1,4-dioxane, 4 mL) at room temperature. The resulting mixture was stirred for 4 h at room temperature. The reaction was monitored by LCMS. The resulting mixture was concentrated under reduced pressure. The resulting mixture was diluted with water (10.00 mL). The mixture was basified to pH 8 with NaOH. The resulting mixture was extracted with EtOAc (3×15 mL) and dried over anhydrous Na₂SO₄. After filtration, the filtrate was concentrated under reduced pressure. The residue was purified by Prep-HPLC with the following conditions: (Column: Xselect CSH C18 OBD Column 30*150 mm 5 μm, n; Mobile Phase A: Water (0.1% FA), Mobile Phase B: MEOH; Flow rate: 60 mL/min mL/min; Gradient: 22% B to 42% B in 9 min; Wave Length: 254 nm/220 nm nm; RT1 (min): 6.35), the pure fraction was concentrated then lyophilized to afford (1S,4S,5S)-5-((3-isopropyl-4-oxopyrido[2′,3′: 3,4]pyrazolo[1,5-a]pyrimidin-1 (4H)-yl)methyl)-2-azabicyclo[2.2.1]heptane-2-sulfonamide.

ESI-MS m/z=417.15 [M+H]⁺; Calculated: 416.2.

¹H NMR (400 MHZ, DMSO-d₆) δ 8.79 (d, J=4.1 Hz, 1H), 8.42 (d, J=8.5 Hz, 1H), 8.15 (s, 1H), 7.17 (dd, J=8.6, 4.2 Hz, 1H), 6.63 (s, 2H), 4.56-4.34 (m, 2H), 3.98 (s, 1H), 3.21-3.00 (m, 2H), 2.67 (d, J=9.2 Hz, 1H), 2.41 (s, 1H), 2.29 (d, J=9.0 Hz, 1H), 1.98 (t, J=10.3 Hz, 1H), 1.81 (d, J=10.2 Hz, 1H), 1.64 (d, J=10.3 Hz, 1H), 1.41 (d, J=11.3 Hz, 1H), 1.35-1.21 (m, 6H).

Total proton count from structure: 24.

Total proton count from spectrum: 24.

### Compound 42: (1S,4S,5S)-5-((3-Isopropyl-7-methoxy-4-oxopyrimido[1,2-b]indazol-1 (4H)-yl)methyl)-2-azabicyclo[2.2.1]heptane-2-sulfonamide

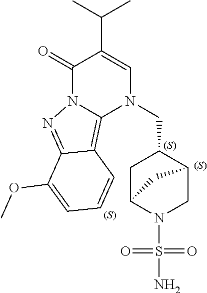

### Step 1: Tert-butyl(1R,4S,5S)-5-formyl-2-azabicyclo[2.2.1]heptane-2-carboxylate

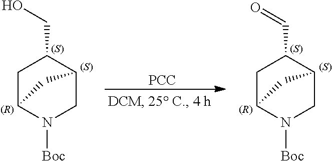

A solution of tert-butyl(1R,4S,5S)-5-(hydroxymethyl)-2-azabicyclo[2.2.1]heptane-2-carboxylate (100 mg, 0.44 mmol, 1.0 equiv) and PCC (1.90 g, 8.79 mmol, 2.0 equiv) in DCM (10 mL) was stirred at 25° C. for 4 h under nitrogen atmosphere. The desired product could be detected by LCMS. The resulting mixture was diluted with DCM (30 mL). The reaction was quenched with NaHSO₃ at 0° C. To the above mixture was added silica gel. The resulting mixture was stirred for 10 min. The resulting mixture was filtered, the filter cake was washed with DCM (3×30 mL). The filtrate was concentrated under reduced pressure. The residue was purified by silica gel column chromatography, eluted with PE/THF (5:1-3:1) to afford tert-butyl(1R,4S,5S)-5-formyl-2-azabicyclo[2.2.1]heptane-2-carboxylate.

ESI-MS m/z=170.0 [M-56]⁺; Calculated: 225.1.

¹H NMR (400 MHZ, DMSO-d₆) δ 9.63 (s, 1H), 4.10 (d, J=15.9 Hz, 1H), 3.19 (ddd, J=15.7, 9.6, 3.5 Hz, 1H), 3.01 (dd, J=9.6, 1.5 Hz, 1H), 2.87-2.81 (m, 1H), 2.70 (dd, J=9.2, 5.2 Hz, 1H), 1.91 (dddd, J=10.1, 7.6, 5.1, 2.5 Hz, 1H), 1.63 (dtt, J=12.3, 6.2, 2.4 Hz, 1H), 1.48 (dd, J=15.8, 10.2 Hz, 1H), 1.39 (d, J=4.1 Hz, 9H), 1.20-1.13 (m, 1H).

### Step 2:7-Methoxy-2H-indazol-3-amine

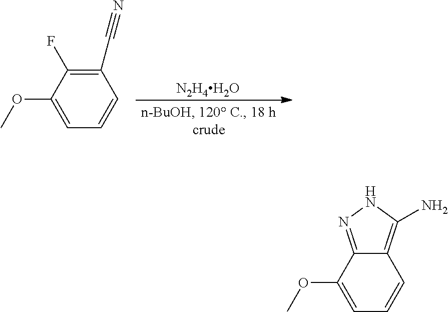

A solution of 2-fluoro-3-methoxybenzonitrile (5.00 g, 33.0 mmol, 1.0 equiv) and hydrazine hydrate (21.97 g, 430.0 mmol, 13.0 equiv, 98% w.t.) in butanol (100 mL) was stirred for 18 h at 120° C. under nitrogen atmosphere. The desired product could be detected by LCMS. The mixture was allowed to cool down to 25° C. The resulting mixture was diluted with water (30 mL). The resulting mixture was extracted with EtOAc (3×50 mL). The combined organic layers were washed with brine (2×10 mL), and dried over anhydrous Na₂SO₄. After filtration, the filtrate was concentrated under reduced pressure. This resulted in 7-methoxy-2H-indazol-3-amine (crude).

ESI-MS m/z=164.1 [M+H]⁺; Calculated: 163.1.

### Step 3: Tert-butyl(1S,4S,5S)-5-(((7-methoxy-2H-indazol-3-yl)amino)methyl)-2-azabicyclo[2.2.1]heptane-2-carboxylate

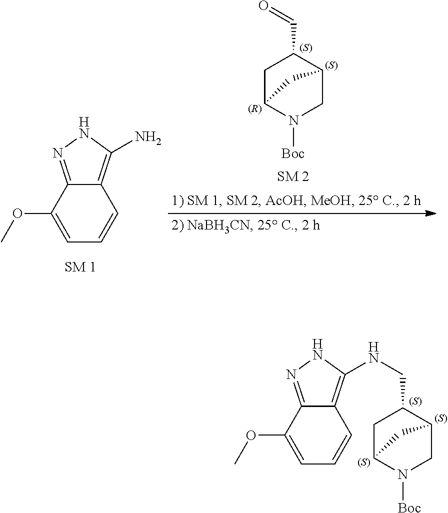

To a stirred solution of 7-methoxy-2H-indazol-3-amine (108 mg, 0.66 mmol, 1.0 equiv) and tert-butyl(1R,4S,5S)-5-formyl-2-azabicyclo[2.2.1]heptane-2-carboxylate (150 mg, 0.66 mmol, 1.0 equiv) in MeOH (2 mL) was added AcOH (4 mg, 0.06 mmol, 0.10 equiv) dropwise at 25° C. under nitrogen atmosphere. The resulting mixture was stirred at 25° C. for 2 h under nitrogen atmosphere. To the above mixture was added NaBH₃CN (83 mg, 1.33 mmol, 2.0 equiv) in portions at 25° C. The resulting mixture was stirred at 25° C. for an additional 2 h. The desired product could be detected by LCMS. The reaction was quenched with water at 0° C. The residue was dissolved in water (2 mL). The resulting mixture was extracted with EtOAc (3×10 mL). The combined organic layers were washed with brine (2×5 mL), and dried over anhydrous Na₂SO₄. After filtration, the filtrate was concentrated under reduced pressure. The residue was purified by reversed-phase flash chromatography with the following conditions: column, C18; mobile phase, MeCN in Water (0.1% TFA), 30% to 40% gradient in 10 min; detector, UV 254 nm.

This resulted in tert-butyl(1S,4S,5S)-5-(((7-methoxy-2H-indazol-3-yl)amino)methyl)-2-azabicyclo[2.2.1]heptane-2-carboxylate.

ESI-MS m/z=373.1 [M+H]⁺; Calculated: 372.2.

### Step 4: 1-[(1R,4S,5S)-2-azabicyclo[2.2.1]heptan-5-ylmethyl]-3-isopropyl-7-methoxypyrimido[1,2-b]indazol-4-one acetic acid salt

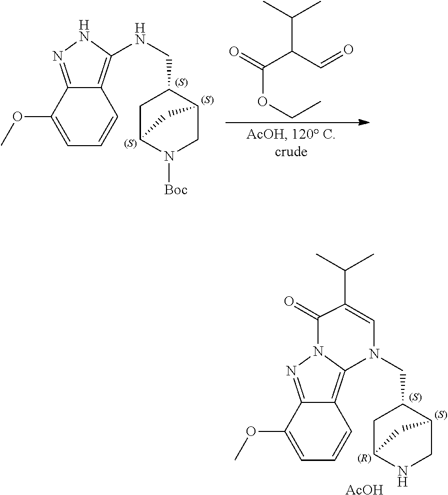

A solution of tert-butyl(1S,4S,5S)-5-(((7-methoxy-2H-indazol-3-yl)amino)methyl)-2-azabicyclo[2.2.1]heptane-2-carboxylate (100 mg, 0.26 mmol, 1.0 equiv) and ethyl 2-formyl-3-methylbutanoate (50 mg, 0.32 mmol, 1.2 equiv) in AcOH (2 mL) was stirred at 120° C. for 2 h under concentrated under reduced pressure. The residue was purified by trituration with Et₂O (3×3 mL). This resulted in 1-[(1R,4S,5S)-2-azabicyclo[2.2.1]heptan-5-ylmethyl]-3-isopropyl-7-methoxypyrimido[1,2-b]indazol-4-one acetic acid salt (crude).

ESI-MS m/z=367.2 [M+H]⁺; Calculated: 366.2.

### Step 5: (1S,4S,5S)-5-((3-isopropyl-7-methoxy-4-oxopyrimido[1,2-b]indazol-1 (4H)-yl)methyl)-2-azabicyclo[2.2.1]heptane-2-sulfonamide

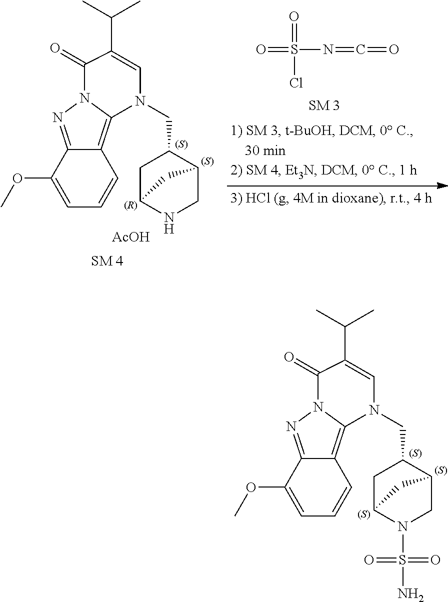

To a stirred solution of chlorosulfonyl isocyanate (341 mg, 2.41 mmol, 6.0 equiv) in DCM (2 mL) was added tert-butanol (267 mg, 3.61 mmol, 9.0 equiv) at 0° C. under nitrogen atmosphere. The resulting mixture was stirred for 30 min at 0° C. under nitrogen atmosphere. A solution of 1-[(1R,4S,5S)-2-azabicyclo[2.2.1]heptan-5-ylmethyl]-3-isopropyl-7-methoxypyrimido[1,2-b]indazol-4-one acetic acid salt (90 mg, 0.24 mmol, 1.0 equiv) in DCM (2 mL) was treated with Et₃N (74 mg, 0.73 mmol, 3.0 equiv) for 5 min at room temperature under nitrogen atmosphere followed by the addition of the above mixture dropwise at 0° C. The resulting mixture was stirred for 1 h at room temperature under nitrogen atmosphere. The reaction was monitored by LCMS. To the above mixture was added HCl (gas, 4 M in 1,4-dioxane, 3 mL) at room temperature. The resulting mixture was stirred for 4 h at room temperature. The reaction was monitored by LCMS. The resulting mixture was concentrated under reduced pressure. The resulting mixture was diluted with water (10 mL). The mixture was basified to pH 8 with NaOH. The resulting mixture was extracted with EtOAc (3×20 mL), and dried over anhydrous Na₂SO₄. After filtration, the filtrate was concentrated under reduced pressure. The residue was purified by Prep-HPLC with the following conditions: (Column: XSelect CSH Fluoro Phenyl 30*150 mm, 5 m; Mobile Phase A: Water (0.1% FA), Mobile Phase B: MeOH; Flow rate: 60 mL/min mL/min; Gradient: 44% B to 61% B in 10 min; Wave Length: 254 nm/220 nm nm; RT1 (min): 8.07/9.38), the pure fraction was concentrated then lyophilized to afford (1S,4S,5S)-5-((3-isopropyl-7-methoxy-4-oxopyrimido[1,2-b]indazol-1 (4H)-yl)methyl)-2-azabicyclo[2.2.1]heptane-2-sulfonamide.

ESI-MS m/z=446.30 [M+H]⁺; Calculated: 445.2.

¹H NMR (400 MHZ, DMSO-d₆) δ 8.08 (s, 1H), 7.47 (d, J=8.6 Hz, 1H), 7.04 (dd, J=8.7, 7.4 Hz, 1H), 6.84 (d, J=7.4 Hz, 1H), 6.63 (s, 2H), 4.47-4.33 (m, 2H), 3.97 (d, J=7.3 Hz, 4H), 3.20-3.11 (m, 1H), 3.07 (dd, J=9.2, 3.6 Hz, 1H), 2.63 (d, J=9.2 Hz, 1H), 2.40 (s, 1H), 2.29 (s, 1H), 1.95 (t, J=10.8 Hz, 1H), 1.79 (t, J=9.5 Hz, 1H), 1.68-1.61 (m, 1H), 1.41 (d, J=11.5 Hz, 1H), 1.27 (dd, J=6.9, 2.4 Hz, 6H).

Total proton count from structure: 27.

Total proton count from spectrum: 27.

### Compound 43: (1S,4S,5S)-5-((3-Fluoro-2,6-diisopropyl-7-oxopyrazolo[1,5-a]pyrimidin-4(7H)-yl)methyl)-2-azabicyclo[2.2.1]heptane-2-sulfonamide

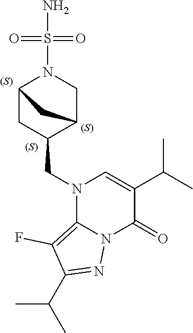

### Step 1: 2,6-Diisopropylpyrazolo[1,5-a]pyrimidin-7(4H)-one

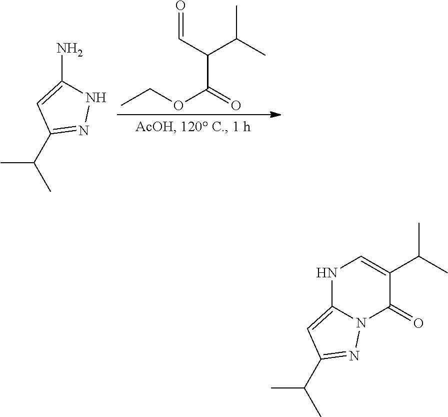

To a stirred mixture of 5-isopropyl-2H-pyrazol-3-amine (2.50 g, 20.0 mmol, 1.0 equiv) in AcOH (50 mL) was added ethyl 2-formyl-3-methylbutanoate (3.16 g, 20.0 mmol, 1.0 equiv) at 25° C. under air atmosphere. The resulting mixture was stirred at 120° C. for 1 h under nitrogen atmosphere. The desired product could be detected by LCMS. The mixture was allowed to cool down to 25° C. The product was precipitated by the addition of water. The precipitated solids were collected by filtration and washed with water (3×10 mL) to afford 2,6-diisopropylpyrazolo[1,5-a]pyrimidin-7(4H)-one.

ESI-MS m/z=220.05 [M+H]⁺; Calculated: 219.3.

¹H NMR (400 MHZ, DMSO-d₆) δ 12.00 (s, 1H), 7.61 (s, 1H), 5.93 (s, 1H), 3.03-2.94 (m, 2H), 1.24 (d, J=6.9 Hz, 6H), 1.17 (d, J=6.9 Hz, 6H).

### Step 2: 2,6-Diisopropyl-4-((2-(trimethylsilyl) ethoxy)methyl) pyrazolo[1,5-a]pyrimidin-7(4H)-one

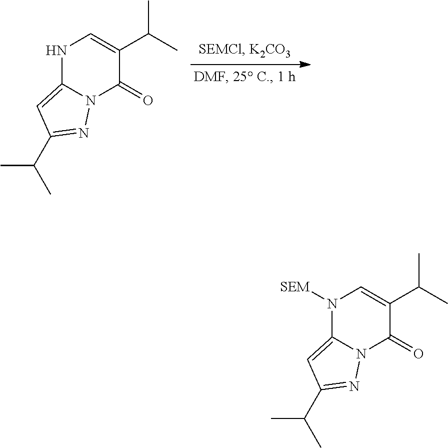 [SMILES: CC(C)c1cc2[nH]cc(C(C)C)c(=O)n2n1 ; CC(C)c1cc2n(COCC[Si](C)(C)C)cc(C(C)C)c(=O)n2n1]

To a stirred mixture of 2,6-diisopropylpyrazolo[1,5-a]pyrimidin-7(4H)-one (4.00 g, 18.2 mmol, 1.0 equiv) in DMF (150 mL) was added K₂CO₃ (7.56 g, 54.7 mmol, 3.0 equiv) and [2-(chloromethoxy)ethyl]-trimethylsilane (3.04 g, 18.2 mmol, 1.0 equiv) at 25° C. under air atmosphere. The resulting mixture was stirred at 25° C. for 1 h under nitrogen atmosphere. The desired product could be detected by LCMS. The resulting mixture was diluted with water (100 mL). The resulting mixture was extracted with EtOAc (3×20 mL). The combined organic layers were washed with brine (1×20 mL), and dried over anhydrous Na₂SO₄. After filtration, the filtrate was concentrated under reduced pressure. The residue was purified by silica gel column chromatography, eluted with PE/EA (5:1-3:1) to afford 2,6-diisopropyl-4-((2-(trimethylsilyl) ethoxy)-methyl) pyrazolo[1,5-a]pyrimidin-7(4H)-one.

ESI-MS m/z=350.10 [M+H]⁺; Calculated: 349.6.

¹H NMR (400 MHZ, DMSO-d₆) δ 7.82 (s, 1H), 6.21 (s, 1H), 5.38 (s, 2H), 3.56 (t, J=7.9 Hz, 2H), 3.05-2.96 (m, 2H), 1.25 (d, J=7.0 Hz, 6H), 1.18 (d, J=6.9 Hz, 6H), 0.86 (t, J=7.9 Hz, 2H), −0.07 (s, 9H).

### Step 3: 3-Fluoro-2,6-diisopropyl-4-((2-(trimethylsilyl) ethoxy)methyl) pyrazolo[1,5-a]pyrimidin-7(4H)-one

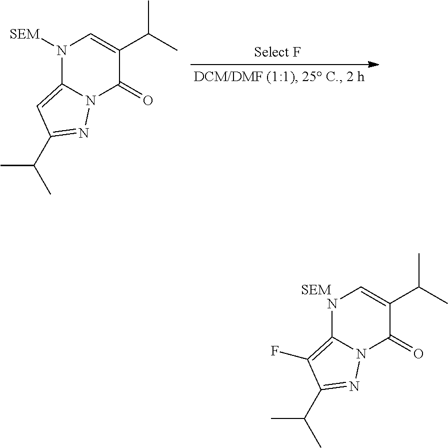 [SMILES: CC(C)c1cc2n(COCC[Si](C)(C)C)cc(C(C)C)c(=O)n2n1 ; CC(C)c1nn2c(=O)c(C(C)C)c[nH]c2c1F ; "SEM"]

To a stirred solution of 2,6-diisopropyl-4-((2-(trimethylsilyl) ethoxy)methyl) pyrazolo[1,5-a]pyrimidin-7(4H)-one (1.50 g, 4.29 mmol, 1.0 equiv) in DCM/DMF (1:1, 30 mL) was added Selectfluor (1.67 g, 4.72 mmol, 1.1 equiv) at 25° C. under air atmosphere. The resulting mixture was stirred at 25° C. for 2 h under nitrogen atmosphere. The desired product could be detected by LCMS. The resulting mixture was diluted with water (100 mL). The resulting mixture was extracted with EtOAc (3×50 mL). The combined organic layers were washed with brine (50 mL), and dried over anhydrous Na₂SO₄. After filtration, the filtrate was concentrated under reduced pressure. The residue was purified by silica gel column chromatography, eluted with PE/EA (5:1-3:1) to afford 3-fluoro-2,6-diisopropyl-4-((2-(trimethylsilyl) ethoxy)methyl) pyrazolo[1,5-a]pyrimidin-7(4H)-one.

ESI-MS m/z=368.15 [M+H]⁺; Calculated: 367.5.

¹H NMR (400 MHZ, DMSO-d₆) δ 7.87 (s, 1H), 5.42 (s, 2H), 3.59 (t, J=7.8 Hz, 2H), 3.15-3.08 (m, 1H), 3.03-2.96 (m, 1H), 1.29 (d, J=6.9 Hz, 6H), 1.18 (d, J=6.9 Hz, 6H), 0.86 (m, 2H), −0.09 (s, 9H).

### Step 4: 3-Fluoro-2,6-diisopropylpyrazolo[1,5-a]pyrimidin-7(4H)-one

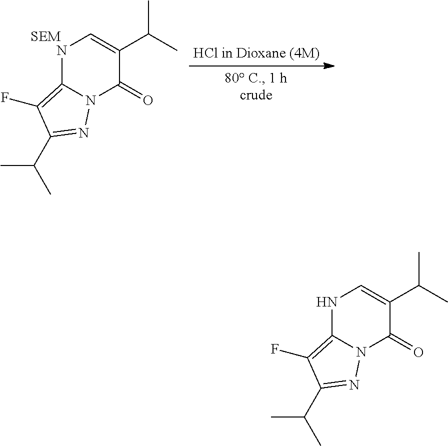 [SMILES: CC(C)c1nn2c(=O)c(C(C)C)c[nH]c2c1F ; "SEM" ; CC(C)c1nn2c(=O)c(C(C)C)c[nH]c2c1F]

To a stirred mixture of 3-fluoro-2,6-diisopropyl-4-((2-(trimethylsilyl) ethoxy)methyl) pyrazolo[1,5-a]pyrimidin-7(4H)-one (450 mg, 1.22 mmol, 1.0 equiv) in HCl (gas, 4 M in 1,4-dioxane, 5 mL) at 25° C. under air atmosphere. The resulting mixture was stirred at 80° C. for 1 h under nitrogen atmosphere. The desired product could be detected by LCMS. The resulting mixture was concentrated under reduced pressure. The precipitated solids were collected by filtration and washed with diethyl ether (2×10 mL) to afford crude product 3-fluoro-2,6-diisopropylpyrazolo[1,5-a]pyrimidin-7(4H)-one (crude). The crude product was used in the next step directly without further purification.

ESI-MS m/z=237.95 [M+H]⁺; Calculated: 237.3.

### Step 5: Tert-butyl(1S,4S,5S)-5-((3-fluoro-2,6-diisopropyl-7-oxopyrazolo[1,5-a]pyrimidin-4(7H)-yl)methyl)-2-azabicyclo[2.2.1]heptane-2-carboxylate

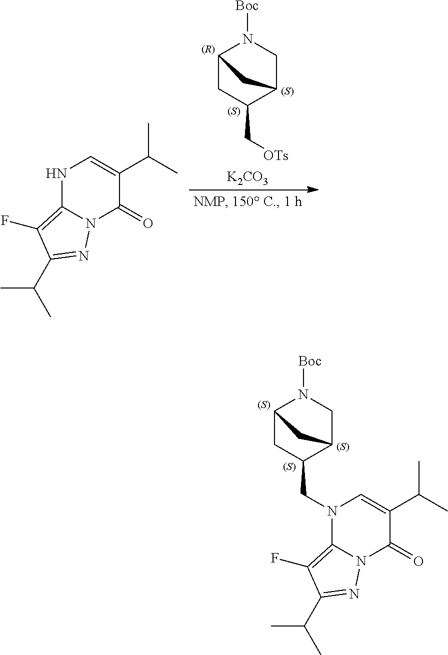

To a stirred mixture of 3-fluoro-2,6-diisopropylpyrazolo[1,5-a]pyrimidin-7(4H)-one (250 mg, 1.05 mmol, 1.0 equiv) in NMP (10 mL) was added K₂CO₃ (437 mg, 3.16 mmol, 3.0 equiv) and tert-butyl(1R,4S,5S)-5-{[(4-methylbenzenesulfonyl)oxy]methyl}-2-azabicyclo[2.2.1]heptane-2-carboxylate (804 mg, 2.11 mmol, 2.0 equiv) at 25° C. under air atmosphere. The resulting mixture was stirred at 150° C. for 1 h under nitrogen atmosphere. The resulting mixture was cooled down to 25° C. The desired products could be detected by LCMS. The resulting mixture was diluted with water (20 mL). The resulting mixture was extracted with EtOAc (3×20 mL). The combined organic layers were washed with brine (1×20 mL), and dried over anhydrous Na₂SO₄. After filtration, the filtrate was concentrated under reduced pressure. The residue was purified by silica gel column chromatography, eluted with PE/EA (4:1-3:1) to afford tert-butyl(1S,4S,5S)-5-((3-fluoro-2,6-diisopropyl-7-oxopyrazolo[1,5-a]pyrimidin-4(7H)-yl)methyl)-2-azabicyclo[2.2.1]heptane-2-carboxylate.

ESI-MS m/z=447.25 [M+H]+; Calculated: 446.3.

### Step 6: 4-(((1R,4S,5S)-2-Azabicyclo[2.2.1]heptan-5-yl)methyl)-3-fluoro-2,6-diisopropylpyrazolo[1,5-a]pyrimidin-7(4H)-one HCl salt

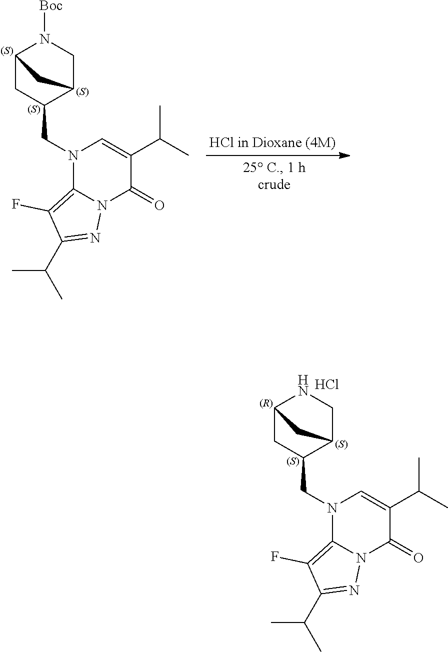

Tert-butyl(1S,4S,5S)-5-((3-fluoro-2,6-diisopropyl-7-oxopyrazolo[1,5-a]pyrimidin-4(7H)-yl)methyl)-2-azabicyclo[2.2.1]heptane-2-carboxylate (300 mg, 0.67 mmol, 1.0 equiv) was stirred in 4M HCl (gas) in 1,4-dioxane (3 mL) at 25° C. under air atmosphere. The resulting mixture was stirred at 25° C. for 1 h under nitrogen atmosphere. The desired product could be detected by LCMS. The resulting mixture was concentrated under reduced pressure to afford crude product 4-(((1R,4S,5S)-2-azabicyclo[2.2.1]heptan-5-yl)methyl)-3-fluoro-2,6-diisopropylpyrazolo[1,5-a]pyrimidin-7(4H)-one HCl salt (crude).

ESI-MS m/z=347.20 [M+H]+; Calculated: 346.2.

### Step 7: (1S,4S,5S)-5-((3-Fluoro-2,6-diisopropyl-7-oxopyrazolo[1,5-a]pyrimidin-4(7H)-yl)methyl)-2-azabicyclo[2.2.1]heptane-2-sulfonamide

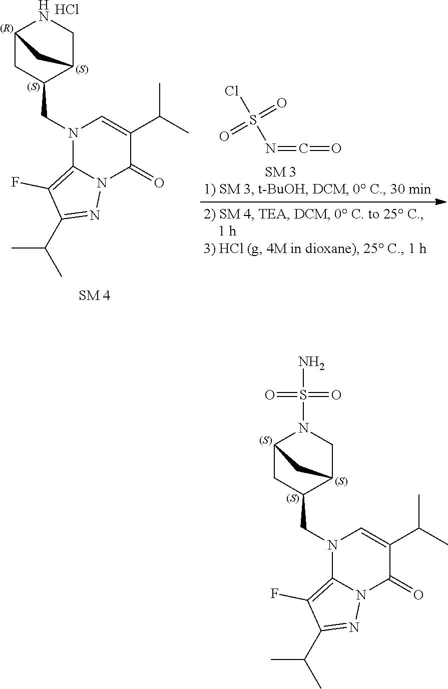

To a stirred solution of chlorosulfonyl isocyanate (810 mg, 5.72 mmol, 6.0 equiv) in DCM (5 mL) was added t-BuOH (735 mg, 9.92 mmol, 9.0 equiv) at 0° C. under nitrogen atmosphere. The resulting mixture was stirred for 30 min at 0° C. under nitrogen atmosphere. A solution of 4-(((1R,4S,5S)-2-azabicyclo[2.2.1]heptan-5-yl)methyl)-3-fluoro-2,6-diisopropylpyrazolo[1,5-a]pyrimidin-7(4H)-one HCl salt (400 mg, 1.15 mmol, 1.0 equiv) in DCM (5 mL) was treated with TEA (584 mg, 5.78 mmol, 5.0 equiv) for 5 min at 25° C. under nitrogen atmosphere. The resulting mixture was stirred for 1 h at 25° C. under nitrogen atmosphere. The reaction was monitored by LCMS. The resulting mixture was concentrated under reduced pressure. To the above mixture was added HCl (gas, 4M in 1,4-dioxane, 5 mL) at 25° C. The resulting mixture was stirred at 25° C. for 1 h under nitrogen atmosphere. The reaction was monitored by LCMS. The resulting mixture was concentrated under vacuum. The crude product (500 mg) was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min mL/min; Gradient: 20% B to 46% B in 7 min; Wave Length: 254 nm/220 nm nm; RT1 (min): 6.2), the pure fraction was concentrated under reduced pressure and then lyophilized to afford (1S,4S,5S)-5-((3-Fluoro-2,6-diisopropyl-7-oxopyrazolo[1,5-a]pyrimidin-4(7H)-yl)methyl)-2-azabicyclo[2.2.1]heptane-2-sulfonamide.

ESI-MS m/z=426.20 [M+H]+; Calculated: 425.2.

¹H NMR (400 MHZ, DMSO-d₆) δ 7.79 (s, 1H), 6.69 (s, 2H), 4.13-3.79 (m, 3H), 3.20-3.06 (m, 2H), 3.06-2.91 (m, 1H), 2.76 (d, J=9.1 Hz, 1H), 2.34-2.29 (m, 1H), 2.24-2.16 (m, 1H), 2.09 (s, 1H), 1.95-1.85 (m, 1H), 1.71-1.60 (m, 2H), 1.30 (d, J=7.0 Hz, 6H), 1.18 (d, J=6.9 Hz, 6H).

¹⁹F NMR (377 MHZ, DMSO-d₆) δ −185.83.

Total proton count from structure: 28.

Total proton count from spectrum: 28.

### Compound 44: (1S,4S,5S)-5-((2-Cyclopropyl-6-isopropyl-3-methoxy-7-oxopyrazolo[1,5-a]pyrimidin-4(7H)-yl)methyl)-2-azabicyclo[2.2.1]heptane-2-sulfonamide

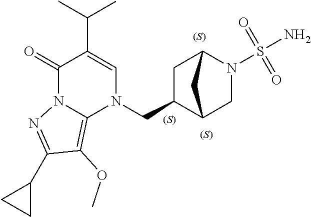

### Step 1: 3-Cyclopropyl-2-methoxy-3-oxopropanenitrile

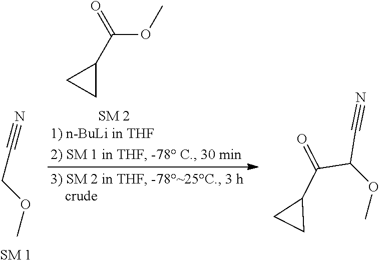

To a stirred mixture of butyllithium (42.2 mL, 105.5 mmol, 1.5 equiv, 2.5 M in hexane) in THF (400 mL) was added 2-methoxyacetonitrile (5.00 g, 70.3 mmol, 1.0 equiv) in THF (50 mL) dropwise at −78° C. under nitrogen atmosphere. The resulting mixture was stirred at −78° C. for 30 min under nitrogen atmosphere. To the above mixture was added methyl cyclopropanecarboxylate (11.27 g, 112.6 mmol, 1.6 equiv) in THF (100 mL) dropwise at −78° C. under nitrogen atmosphere over 30 min. The resulting mixture was allowed to warm to 25° C. and stirred for an additional 3 h under nitrogen atmosphere. The new product could be detected by LCMS. The reaction was quenched with sat. NH₄Cl (aq.) at 25° C. The mixture was acidified to pH 4 with HCl (2N, aq.). The aqueous layer was extracted with EtOAc (3×100 mL). The combined organic layers were washed with brine (1×100 mL), and dried over anhydrous Na₂SO₄. After filtration, the filtrate was concentrated under reduced pressure to afford crude product 3-cyclopropyl-2-methoxy-3-oxopropanenitrile. The crude product was used in the next step directly without further purification.

ESI-MS m/z=137.95 [M−H]⁻; Calculated: 139.1.

### Step 2: 3-Cyclopropyl-4-methoxy-1H-pyrazol-5-amine

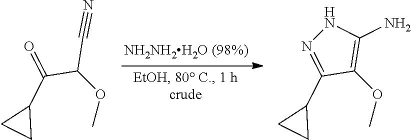

To a stirred mixture of 3-cyclopropyl-2-methoxy-3-oxopropanenitrile (10.00 g, 71.9 mmol, 1.0 equiv) in EtOH (180 mL) was added NH₂OH·H₂O (7.34 g, 143.7 mmol, 2.0 equiv, 98 wt %) at 25° C. under nitrogen atmosphere. The resulting mixture was stirred at 80° C. for 1 h under nitrogen atmosphere. The desired product could be detected by LCMS. The mixture was allowed to cool down to 25° C. The resulting mixture was diluted with water (100 mL). The resulting mixture was extracted with CHCl3/IPA (5:1) (3×200 mL). The combined organic layers were washed with brine (1×100 mL), and dried over anhydrous Na₂SO₄. After filtration, the filtrate was concentrated under reduced pressure to afford crude product 3-cyclopropyl-4-methoxy-1H-pyrazol-5-amine. The crude product was used in the next step directly without further purification.

ESI-MS m/z=154.10 [M+H]⁺; Calculated: 153.1.

### Step 3: 2-Cyclopropyl-6-isopropyl-3-methoxypyrazolo[1,5-a]pyrimidin-7(4H)-one

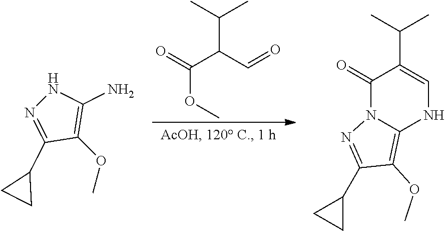

To a stirred mixture of 3-cyclopropyl-4-methoxy-1H-pyrazol-5-amine (1.00 g, 6.5 mmol, 1.0 equiv) in AcOH (15 mL) was added ethyl 2-formyl-3-methylbutanoate (1.03 g, 6.5 mmol, 1.0 equiv) at 25° C. under air atmosphere. The resulting mixture was stirred at 120° C. for 1 h under nitrogen atmosphere. The desired product could be detected by LCMS. The mixture was allowed to cool down to 25° C. The resulting mixture was diluted with water (50 mL). The resulting mixture was extracted with EtOAc (3×20 mL). The combined organic layers were washed with brine (1×20 mL), and dried over anhydrous Na₂SO₄. After filtration, the filtrate was concentrated under reduced pressure. The residue was purified by silica gel column chromatography, eluted with CH₂Cl₂/MeOH (10:1) to afford 2-cyclopropyl-6-isopropyl-3-methoxypyrazolo[1,5-a]pyrimidin-7(4H)-one.

ESI-MS m/z=248.00 [M+H]⁺; Calculated: 247.1.

¹H NMR (400 MHZ, DMSO-d₆) δ 12.10 (s, 1H), 7.49 (d, J=0.7 Hz, 1H), 3.78 (s, 3H), 3.01-2.91 (m, 1H), 2.01-1.95 (m, 1H), 1.15 (d, J=6.9 Hz, 6H), 0.99-0.93 (m, 2H), 0.90-0.88 (m, 2H).

### Step 4: Tert-butyl(1S,4S,5S)-5-((2-cyclopropyl-6-isopropyl-3-methoxy-7-oxopyrazolo[1,5-a]pyrimidin-4(7H)-yl)methyl)-2-azabicyclo[2.2.1]heptane-2-carboxylate

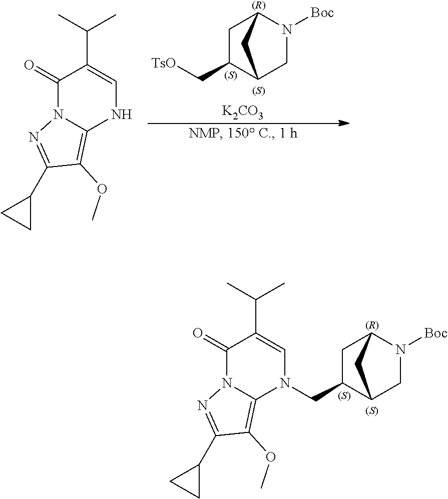

To a stirred mixture of 2-cyclopropyl-6-isopropyl-3-methoxypyrazolo[1,5-a]pyrimidin-7(4H)-one (200 mg, 0.81 mmol, 1.0 equiv) in NMP (8 mL) was added K₂CO₃ (335 mg, 2.43 mmol, 3.0 equiv) and tert-butyl(1R,4S,5S)-5-{[(4-methylbenzenesulfonyl)oxy]methyl}-2-azabicyclo[2.2.1]heptane-2-carboxylate (85 mg, 0.22 mmol, 1.1 equiv) at 25° C. under air atmosphere. The resulting mixture was stirred at 150° C. for 1 h under nitrogen atmosphere. The desired product could be detected by LCMS. The mixture was allowed to cool down to 25° C. The resulting mixture was diluted with water (50 mL). The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EA (1:1) to afford tert-butyl(1S,4S,5S)-5-((2-cyclopropyl-6-isopropyl-3-methoxy-7-oxopyrazolo[1,5-a]pyrimidin-4(7H)-yl)methyl)-2-azabicyclo[2.2.1]heptane-2-carboxylate.

ESI-MS m/z=457.25 [M+H]⁺; Calculated: 456.3.

### Step 5: 4-(((1R,4S,5S)-2-Azabicyclo[2.2.1]heptan-5-yl)methyl)-2-cyclopropyl-6-isopropyl-3-methoxypyrazolo[1,5-a]pyrimidin-7(4H)-one HCl salt

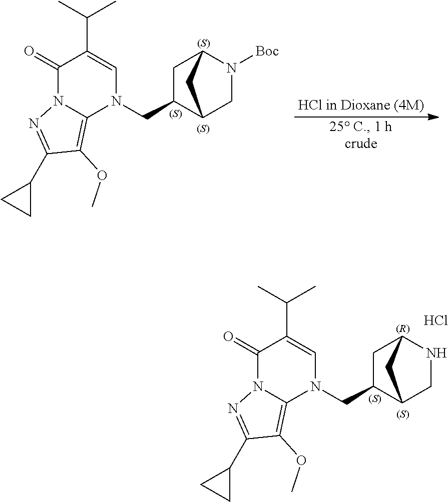

Tert-butyl(1S,4S,5S)-5-((2-cyclopropyl-6-isopropyl-3-methoxy-7-oxopyrazolo[1,5-a]pyrimidin-4(7H)-yl)methyl)-2-azabicyclo[2.2.1]heptane-2-carboxylate (230 mg, 0.50 mmol, 1.0 equiv) was stirred in HCl (gas, 4 M in 1,4-dioxane, 5 mL) at 25° C. under air atmosphere. The resulting mixture was stirred at 25° C. for 1 h under nitrogen atmosphere. The desired product could be detected by LCMS. The resulting mixture was concentrated under reduced pressure to afford crude product 4-(((1R,4S,5S)-2-azabicyclo[2.2.1]heptan-5-yl)methyl)-2-cyclopropyl-6-isopropyl-3-methoxypyrazolo[1,5-a]pyrimidin-7(4H)-one HCl salt.

ESI-MS m/z=357.15 [M+H]⁺; Calculated: 356.2.

### Step 6: (1S,4S,5S)-5-((2-Cyclopropyl-6-isopropyl-3-methoxy-7-oxopyrazolo[1,5-a]pyrimidin-4(7H)-yl)methyl)-2-azabicyclo[2.2.1]heptane-2-sulfonamide

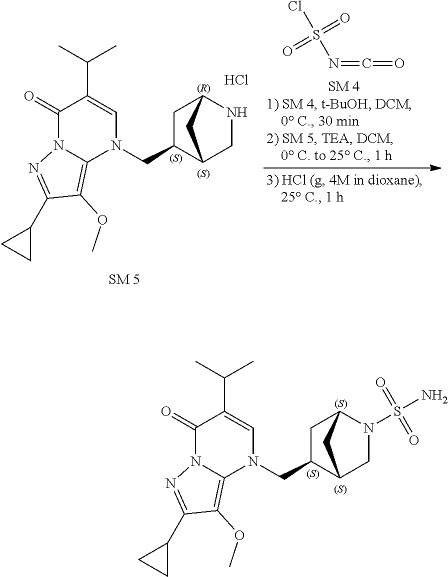

To a stirred solution of chlorosulfonyl isocyanate (394 mg, 2.78 mmol, 5.0 equiv) in DCM (6 mL) was added t-BuOH (357 mg, 4.82 mmol, 8.6 equiv) dropwise at 0° C. The resulting mixture was stirred for 30 min at 0° C. under nitrogen atmosphere. To a stirred solution of 4-(((1R,4S,5S)-2-azabicyclo[2.2.1]heptan-5-yl)methyl)-2-cyclopropyl-6-isopropyl-3-methoxypyrazolo[1,5-a]pyrimidin-7(4H)-one HCl salt (200 mg, 0.56 mmol, 1.0 equiv) in DCM (6 mL) was added TEA (284 mg, 2.81 mmol, 5.0 equiv) at 25° C. under nitrogen atmosphere followed by the addition of the above mixture dropwise at 0° C. The resulting mixture was stirred for 1 h at 0° C. under nitrogen atmosphere. The desired product could be detected by LCMS. The resulting mixture was concentrated under reduced pressure. To the above mixture was added HCl (gas, 4M in 1,4-dioxane, 3 mL) at 25° C. The resulting mixture was stirred for an additional 1 h at 25° C. The desired products could be detected by LCMS. The resulting mixture was concentrated under reduced pressure. The crude product (500 mg) was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min mL/min; Gradient: 20% B to 46% B in 7 min; Wave Length: 254 nm/220 nm nm; RT1 (min): 6.2), the pure fraction was concentrated under reduced pressure then lyophilized to afford (1S,4S,5S)-5-((2-cyclopropyl-6-isopropyl-3-methoxy-7-oxopyrazolo[1,5-a]pyrimidin-4(7H)-yl)methyl)-2-azabicyclo[2.2.1]heptane-2-sulfonamide.

ESI-MS m/z=436.15 [M+H]⁺; Calculated: 435.2.

¹H NMR (400 MHZ, DMSO-d₆) δ 7.70 (s, 1H), 6.68 (s, 2H), 4.07-4.01 (m, 1H), 3.98-3.89 (m, 2H), 3.86 (s, 3H), 3.10 (dd, J=9.2, 3.5 Hz, 1H), 3.02-2.91 (m, 1H), 2.73 (d, J=9.1 Hz, 1H), 2.33-2.27 (m, 1H), 2.26-2.16 (m, 1H), 2.07-1.97 (m, 1H), 1.90 (t, J=10.6 Hz, 1H), 1.71-1.59 (m, 2H), 1.31-1.23 (m, 1H), 1.16 (d, J=6.9 Hz, 6H), 1.05-0.87 (m, 4H).

Total proton count from structure: 29.

Total proton count from spectrum: 29.

### Compound 45: (1S,4S,5S)-5-((3-Cyano-2-cyclopropyl-6-isopropyl-7-oxopyrazolo[1,5-a]pyrimidin-4(7H)-yl)methyl)-2-azabicyclo[2.2.1]heptane-2-sulfonamide

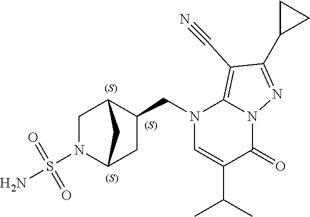

### Step 1: tert-Butyl(1S,4S,5S)-5-((2-cyclopropyl-3-iodo-6-isopropyl-7-oxopyrazolo[1,5-a]pyrimidin-4(7H)-yl)methyl)-2-azabicyclo[2.2.1]heptane-2-carboxylate

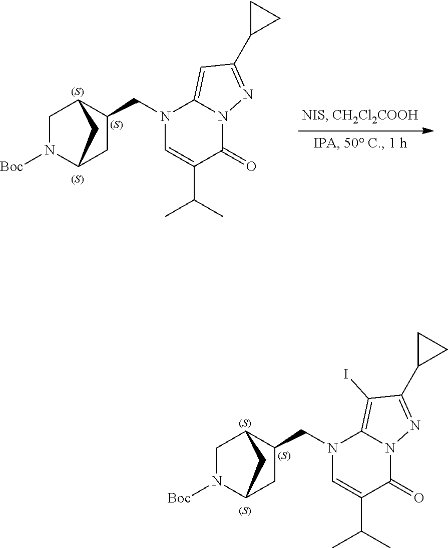

To a stirred solution of tert-butyl(1S,4S,5S)-5-((2-cyclopropyl-6-isopropyl-7-oxopyrazolo[1,5-a]pyrimidin-4(7H)-yl)methyl)-2-azabicyclo[2.2.1]heptane-2-carboxylate (400 mg, 0.94 mmol, 1.0 equiv) and NIS (211 mg, 0.94 mmol, 1.0 equiv) in IPA (8 mL) was added 2,2-dichloroacetic acid (12 mg, 0.09 mmol, 0.1 equiv) at 25° C. The resulting mixture was stirred at 50° C. for 1 h under nitrogen atmosphere. The desired product could be detected by LCMS. The mixture was allowed to cool down to 25° C. The residue was purified by reversed-phase flash chromatography with the following conditions: column, C18; mobile phase, MeCN in Water (0.1% FA), 30% to 60% gradient in 15 min; detector, UV 254 nm to afford tert-butyl(1S,4S,5S)-5-((2-cyclopropyl-3-iodo-6-isopropyl-7-oxopyrazolo[1,5-a]pyrimidin-4(7H)-yl)methyl)-2-azabicyclo[2.2.1]heptane-2-carboxylate.

ESI-MS m/z=553.10 [M+H]⁺; Calculated: 552.2.

### Step 2: tert-butyl(1S,4S,5S)-5-({3-cyano-2-cyclopropyl-6-isopropyl-7-oxopyrazolo[1,5-a]pyrimidin-4-yl}methyl)-2-azabicyclo[2.2.1]heptane-2-carboxylate

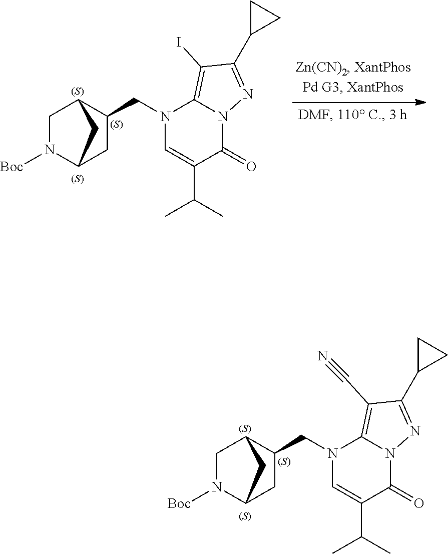

To a stirred solution of tert-butyl(1S,4S,5S)-5-({2-cyclopropyl-3-iodo-6-isopropyl-7-oxopyrazolo[1,5-a]pyrimidin-4-yl}methyl)-2-azabicyclo[2.2.1]heptane-2-carboxylate (50 mg, 0.091 mmol, 1 equiv) and zincdicarbonitrile (191 mg, 1.63 mmol, 3.0 equiv) in DMF (6 mL) were added XantPhos Pd G3 (52 mg, 0.05 mmol, 0.10 equiv) and XantPhos (10 mg, 0.02 mmol, 0.20 equiv) at 25° C. under nitrogen atmosphere. The resulting mixture was stirred at 110° C. for 3 h under nitrogen atmosphere. The desired product could be detected by LCMS. The mixture was allowed to cool down to 25° C. The residue was purified by reversed-phase flash chromatography with the following conditions: column, C18; mobile phase, MeCN in Water (0.1% FA), 40% to 70% gradient in 10 min; detector, UV 254 nm. The resulting mixture was concentrated under reduced pressure to afford tert-butyl(1S,4S,5S)-5-({3-cyano-2-cyclopropyl-6-isopropyl-7-oxopyrazolo[1,5-a]pyrimidin-4-yl}methyl)-2-azabicyclo[2.2.1]heptane-2-carboxylate.

ESI-MS m/z=452.35 [M+H]⁺; Calculated: 451.6.

¹H NMR (400 MHZ, DMSO-d₆) δ 7.88 (s, 1H), 4.21-4.00 (m, 3H), 3.19-3.06 (m, 1H), 2.96 (h, J=7.0 Hz, 1H), 2.91-2.81 (m, 1H), 2.38-2.21 (m, 2H), 2.10-1.95 (m, 1H), 1.82-1.72 (m, 2H), 1.63-1.53 (m, 1H), 1.42-1.28 (m, 10H), 1.18 (d, J=7.3 Hz, 6H), 1.14-1.05 (m, 2H), 1.03-0.96 (m, 2H).

### Step 3: 4-(((1R,4S,5S)-2-azabicyclo[2.2.1]heptan-5-yl)methyl)-2-cyclopropyl-6-isopropyl-7-oxo-4,7-dihydropyrazolo[1,5-a]pyrimidine-3-carbonitrile HCl salt

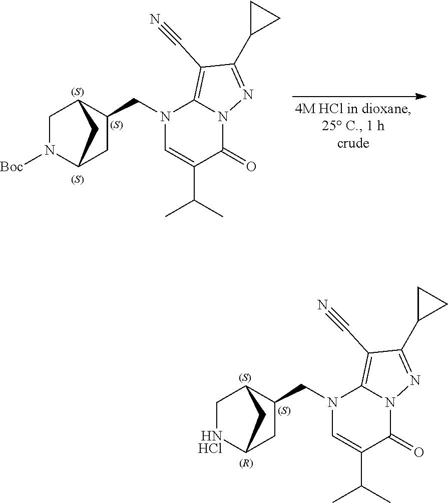

A solution of tert-butyl(1S,4S,5S)-5-({3-cyano-2-cyclopropyl-6-isopropyl-7-oxopyrazolo[1,5-a]pyrimidin-4-yl}methyl)-2-azabicyclo[2.2.1]heptane-2-carboxylate (200 mg, 0.44 mmol, 1.0 equiv) in 4M HCl (gas) in 1,4-dioxane (5 mL) was stirred at 25° C. for 1 h. The desired product could be detected by LCMS. The resulting mixture was concentrated under reduced pressure. This resulted in 4-(((1R,4S,5S)-2-azabicyclo[2.2.1]heptan-5-yl)methyl)-2-cyclopropyl-6-isopropyl-7-oxo-4,7-dihydropyrazolo[1,5-a]pyrimidine-3-carbonitrile HCl salt (crude).

ESI-MS m/z=352.05 [M+H]⁺; Calculated: 351.2.

### Step 4: (1S,4S,5S)-5-((3-cyano-2-cyclopropyl-6-isopropyl-7-oxopyrazolo[1,5-a]pyrimidin-4(7H)-yl)methyl)-2-azabicyclo[2.2.1]heptane-2-sulfonamide

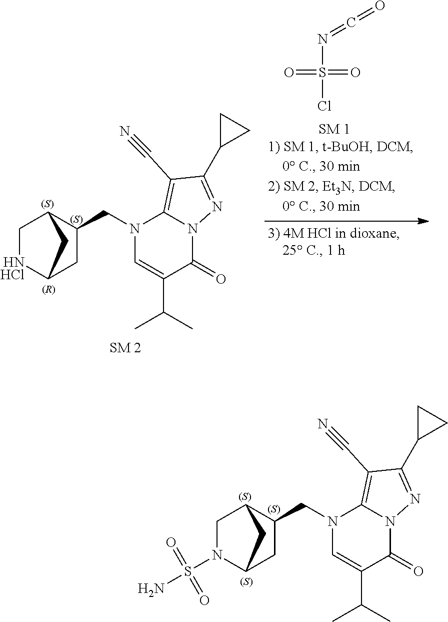

To a stirred solution of chlorosulfonyl isocyanate (657 mg, 4.64 mmol, 6.0 equiv) in DCM (4.8 mL) was added t-BuOH (516 mg, 6.96 mmol, 9.0 equiv) dropwise at 0° C. under nitrogen atmosphere. The resulting mixture was stirred for 30 min at 0° C. under nitrogen atmosphere. A stirred solution of 4-[(1R,4S,5S)-2-azabicyclo[2.2.1]heptan-5-ylmethyl]-2-cyclopropyl-6-isopropyl-7-oxopyrazolo[1,5-a]pyrimidine-3-carbonitrile hydrochloride (300 mg, 0.77 mmol, 1.0 equiv) in DCM (10 mL) was treated with Et₃N (1.41 g, 13.9 mmol, 18.0 equiv) for 5 min at 25° C. under nitrogen atmosphere followed by the addition of the above mixture dropwise at 0° C. The resulting mixture was stirred for 30 min at 0° C. under nitrogen atmosphere. The reaction was monitored by LCMS. The resulting mixture was concentrated under reduced pressure. To the above mixture was added HCl (gas) in 1,4-dioxane (20 mL) over 1 min at 25° C. The resulting mixture was stirred for an additional 1 h at 25° C. The reaction was monitored by LCMS. The resulting mixture was concentrated under reduced pressure. The resulting mixture was diluted with water (50 mL). The mixture was basified to pH 10 with 1M NaOH(aq.). The resulting mixture was extracted with CH₂Cl₂ (3×50 mL), and dried over anhydrous Na₂SO₄. After filtration, the filtrate was concentrated under reduced pressure. The crude product (200 mg) was purified by Prep-HPLC with the following conditions (Column: XBridge Shield RP18 OBD Column 30*150 mm, 5 m; Mobile Phase A: Water (10 mmol/L NH₄HCO₃), Mobile Phase B: ACN; Flow rate: 60 mL/min; Gradient: 52% B to 68% B in 10 min; Wave Length: 254 nm/220 nm; RT1 (min): 9.2), the pure fraction was concentrated then lyophilized to afford (1S,4S,5S)-5-((3-cyano-2-cyclopropyl-6-isopropyl-7-oxopyrazolo[1,5-a]pyrimidin-4(7H)-yl)methyl)-2-azabicyclo[2.2.1]heptane-2-sulfonamide.

ESI-MS m/z=431.05 [M+H]⁺; Calculated: 430.2.

¹H NMR (400 MHZ, DMSO-d₆) δ 7.87 (s, 1H), 6.67 (s, 2H), 4.20-4.03 (m, 2H), 3.96 (s, 1H), 3.15-3.04 (m, 1H), 3.04-2.91 (m, 1H), 2.77 (d, J=9.0 Hz, 1H), 2.37-2.25 (m, 2H), 2.11-2.00 (m, 1H), 2.00-1.90 (m, 1H), 1.77-1.68 (m, 1H), 1.64 (d, J=10.4 Hz, 1H), 1.33-1.24 (m, 1H), 1.23-1.13 (m, 6H), 1.14-1.05 (m, 2H), 1.05-0.96 (m, 2H).

Total proton count from structure: 26.

Total proton count from spectrum: 26.

### Compound 46: 6-Isopropyl-4-(((1S,4S,5S)-2-(S-methylsulfonimidoyl)-2-azabicyclo[2.2.1]heptan-5-yl)methyl)-2-(trifluoromethoxy) pyrazolo[1,5-a]pyrimidin-7(4H)-one

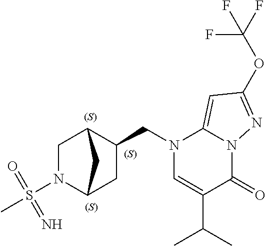

### Step 1: 2-(5-Hydroxy-2H-pyrazol-3-yl) isoindole-1,3-dione

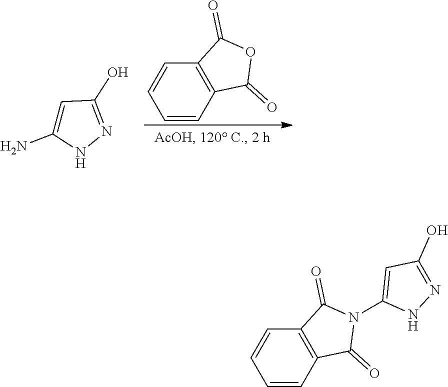

A mixture of 5-amino-1H-pyrazol-3-ol (10.00 g, 100.9 mmol, 1.0 equiv) and phthalic anhydride (14.95 g, 100.9 mmol, 1.0 equiv) in AcOH (120 mL) was stirred for 2 h at 120° C. under nitrogen atmosphere. The desired product could be detected by LCMS. The mixture was allowed to cool down to 25° C. The precipitated solids were collected by filtration and washed with THF (2×5 mL). The residue was purified by trituration with PE/THF (3:1, 2 L). The precipitated solids were collected by filtration and washed with PE (2×50 mL). The resulting mixture was concentrated under reduced pressure to afford 2-(5-hydroxy-2H-pyrazol-3-yl) isoindole-1,3-dione.

ESI-MS m/z=230.05 [M+H]⁺; Calculated: 229.0.

¹H NMR (400 MHZ, DMSO-d₆) δ 12.21 (s, 1H), 10.76 (s, 1H), 7.98-7.92 (m, 2H), 7.92-7.86 (m, 2H), 5.51 (s, 1H).

### Step 2: 2-[5-(Trifluoromethoxy)-2H-pyrazol-3-yl]isoindole-1,3-dione

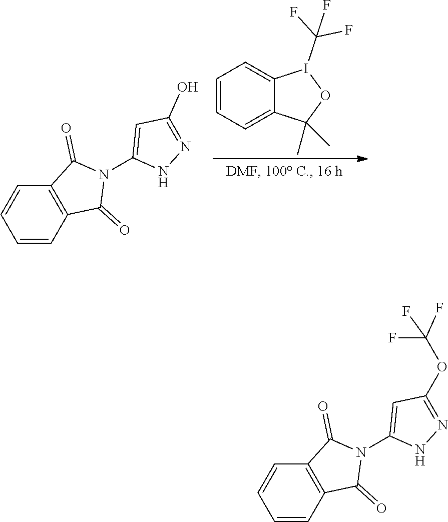

A mixture of 2-(5-hydroxy-2H-pyrazol-3-yl) isoindole-1,3-dione (13.00 g, 56.7 mmol, 1.0 equiv) and 3,3-dimethyl-1-(trifluoromethyl)-1lambda3,2-benziodaoxole (22.47 g, 68.0 mmol, 1.2 equiv) in DMF (130 mL) was stirred at 100° C. for 16 h under nitrogen atmosphere. The desired product could be detected by LCMS. The mixture was allowed to cool down to 25° C. The resulting mixture was diluted with water (650 mL). The resulting mixture was extracted with EtOAc (3×500 mL). The combined organic layers were washed with brine (2×500 mL), and dried over anhydrous Na₂SO₄. After filtration, the filtrate was concentrated under reduced pressure. The crude product was purified by silica gel column chromatography, eluted with EA in PE (0% to 30% gradient in 20 min, hold at 30%) to afford the crude product. The crude product was purified by reverse phase flash with the following conditions (column, C18; mobile phase, MeCN in Water (10 mmol/L NH₄HCO₃), 40% to 90% gradient in 25 min, hold at 69%; detector, UV 254 nm/220 nm) to afford 2-[5-(trifluoromethoxy)-2H-pyrazol-3-yl]isoindole-1,3-dione.

ESI-MS m/z=297.95 [M+H]⁺; Calculated: 297.0.

¹H NMR (400 MHZ, DMSO-d₆) δ 13.38 (s, 1H), 8.07-7.99 (m, 2H), 7.99-7.92 (m, 2H), 6.40 (s, 1H).

¹⁹F NMR (376 MHZ, DMSO-d₆) δ −58.07.

### Step 3: 5-(Trifluoromethoxy)-2H-pyrazol-3-amine

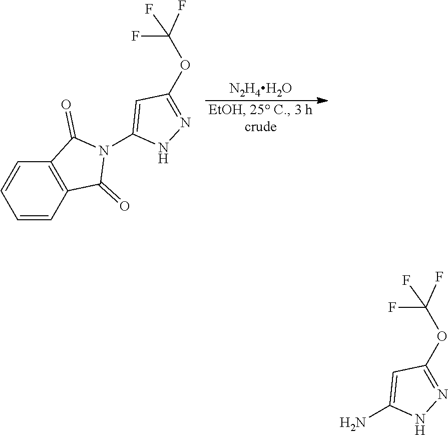 [SMILES: O=C1c2ccccc2C(=O)N1c1cc(OC(F)(F)F)n[nH]1 ; "N2H4·H2O" ; "EtOH, 25° C., 3 hcrude" ; Nc1cc(OC(F)(F)F)n[nH]1]

A mixture of 2-[5-(trifluoromethoxy)-2H-pyrazol-3-yl]isoindole-1,3-dione (700 mg, 2.35 mmol, 1.0 equiv) and hydrazine hydrate (98% wt, 1.18 g, 23.5 mmol, 10.0 equiv) in EtOH (10 mL) was stirred at 25° C. for 3 h. The desired product could be detected by LCMS. The reaction was diluted with Water (50 ml) at 0° C. The aqueous layer was extracted with EtOAc (3×50 mL). The resulting mixture was concentrated under vacuum to afford 5-(trifluoromethoxy)-2H-pyrazol-3-amine (crude).

ESI-MS m/z=168.00 [M+H]⁺; Calculated: 167.0.

¹H NMR (400 MHZ, DMSO-d₆) δ 11.31 (s, 1H), 5.31 (s, 2H), 5.14-5.01 (m, 1H).

¹⁹F NMR (376 MHZ, DMSO) δ −57.77.

### Step 4: 6-Isopropyl-2-(trifluoromethoxy)-4H-pyrazolo[1,5-a]pyrimidin-7-one

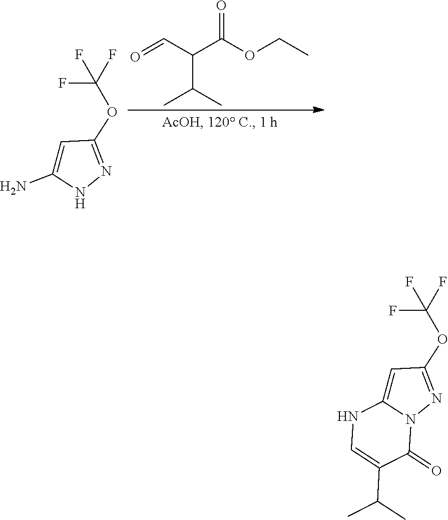

A mixture of 5-(trifluoromethoxy)-2H-pyrazol-3-amine (400 mg, 2.39 mmol, 1.0 equiv) and ethyl 2-formyl-3-methylbutanoate (454 mg, 2.87 mmol, 1.2 equiv) in AcOH (8 mL) was stirred at 120° C. for 1 h. The desired product could be detected by LCMS. The mixture was allowed to cool down to 25° C. The reaction was diluted with water (80 mL) at 25° C. The precipitated solids were collected by filtration and washed with water (2×10 mL) The resulting mixture was concentrated under reduced pressure to afford 6-isopropyl-2-(trifluoromethoxy)-4H-pyrazolo[1,5-a]pyrimidin-7-one.

ESI-MS m/z=262.15 [M+H]⁺; Calculated: 261.0.

¹H NMR (400 MHZ, DMSO-d₆) δ 12.39 (s, 1H), 7.74 (s, 1H), 6.04 (s, 1H), 3.05-2.93 (m, 1H), 1.18 (d, J=7.0 Hz, 6H).

¹⁹F NMR (376 MHZ, DMSO) δ −57.68.

### Step 5: Tert-butyl(1S,4S,5S)-5{[6-isopropyl-7-oxo-2-(trifluoromethoxy) pyrazolo[1,5-a]pyrimidin-4-yl]methyl}-2-azabicyclo[2.2.1]heptane-2-carboxylate

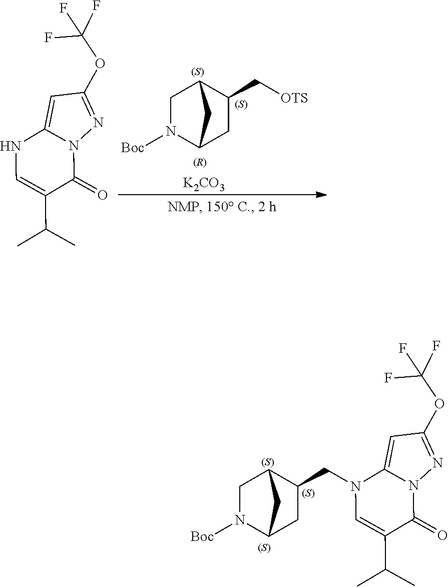

A mixture of 6-isopropyl-2-(trifluoromethoxy)-4H-pyrazolo[1,5-a]pyrimidin-7-one (400 mg, 1.53 mmol, 1.0 equiv), K₂CO₃ (634 mg, 4.59 mmol, 3.0 equiv) and tert-butyl(1R,4S,5S)-5-{[(4-methylbenzenesulfonyl)oxy]methyl}-2-azabicyclo[2.2.1]heptane-2-carboxylate (642 mg, 1.68 mmol, 1.1 equiv) in NMP (6 mL) was stirred at 150° C. for 2 h. The desired product could be detected by LCMS. The mixture was allowed to cool down to 25° C. The residue was purified by reversed-phase flash chromatography with the following conditions: (column, C18; mobile phase, MeCN in Water (10 mmol/L NH₄HCO₃), 30% to 70% gradient in 15 min; detector, UV 254 nm) to afford tert-butyl(1S,4S,5S)-5{[6-isopropyl-7-oxo-2-(trifluoromethoxy) pyrazolo[1,5-a]pyrimidin-4-yl]methyl}-2-azabicyclo[2.2.1]heptane-2-carboxylate.

ESI-MS m/z=471.25 [M+H]⁺; Calculated: 470.2.

¹H NMR (400 MHZ, DMSO-d₆) δ 7.88 (s, 1H), 6.53 (s, 1H), 4.13-3.93 (m, 2H), 3.93-3.81 (m, 1H), 3.18-3.06 (m, 1H), 3.05-2.93 (m, 1H), 2.92-2.81 (m, 1H), 2.34-2.18 (m, 2H), 1.88-1.76 (m, 1H), 1.75-1.61 (m, 1H), 1.58-1.45 (m, 1H), 1.36 (d, J=6.6 Hz, 9H), 1.30-1.23 (m, 1H), 1.19 (d, J=6.9 Hz, 6H).

¹⁹F NMR (377 MHZ, DMSO) δ −57.60.

### Step 6: 4-[(1R,4S,5S)-2-azabicyclo[2.2.1]heptan-5-ylmethyl]-6-isopropyl-2-(trifluoromethoxy) pyrazolo[1,5-a]pyrimidin-7-one

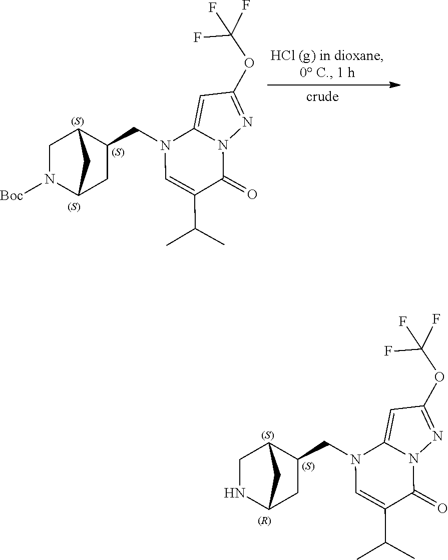

A solution of tert-butyl(1S,4S,5S)-5{[6-isopropyl-7-oxo-2-(trifluoromethoxy) pyrazolo[1,5-a]pyrimidin-4-yl]methyl}-2-azabicyclo[2.2.1]heptane-2-carboxylate (450 mg, 0.106 mmol, 1.0 equiv) in 4M HCl (gas) in 1,4-dioxane (5 mL) was stirred at 0° C. for 1 h. The desired product could be detected by LCMS. The reaction was quenched with Water (100 mL) at 25° C. The aqueous layer was extracted with DCM/IPA (10:1, 3×100 mL). The resulting mixture was concentrated under vacuum to afford 4-[(1R,4S,5S)-2-azabicyclo[2.2.1]heptan-5-ylmethyl]-6-isopropyl-2-(trifluoromethoxy) pyrazolo[1,5-a]pyrimidin-7-one.

ESI-MS m/z=371.05 [M+H]⁺; Calculated: 370.2

### Step 7: 4-{[(1S,4S,5S)-2-{[(tert-butyldimethylsilyl)imino](methyl)oxo-lambda6-sulfanyl}-2-azabicyclo[2.2.1]heptan-5-yl]methyl}-6-isopropyl-2-(trifluoromethoxy) pyrazolo[1,5-a]pyrimidin-7-one

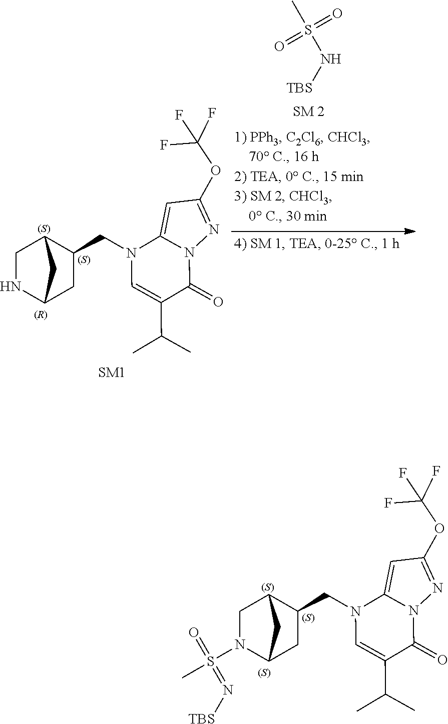

A solution of PPh₃ (1.27 g, 4.86 mmol, 6.0 equiv) and 1,1,1,2,2,2-hexachloroethane (1.15 g, 4.86 mmol, 6.0 equiv) in CHCl3 (15 mL) was stirred for 16 h at 70° C. under nitrogen atmosphere. The mixture was allowed to cool down to 0° C. To the above mixture was added TEA (740 mg, 7.29 mmol, 9.0 equiv) dropwise at 0° C. The resulting mixture was stirred for an additional 15 min at 0° C. To the above mixture was added N-(tert-butyldimethylsilyl)methanesulfonamide (1.02 g, 4.86 mmol, 6.0 equiv) in CHCl3 (10 mL) dropwise at 0° C. The resulting mixture was stirred for an additional 30 min at 0° C. To the above mixture was added 4-[(1R,4S,5S)-2-azabicyclo[2.2.1]heptan-5-ylmethyl]-6-isopropyl-2-(trifluoromethoxy) pyrazolo[1,5-a]pyrimidin-7-one (300 mg, 0.810 mmol, 1.0 equiv) in CHCl3 (5 mL) and TEA (410 mg, 4.05 mmol, 5.0 equiv) dropwise at 0° C. The resulting mixture was stirred for an additional 1 h at 25° C. The desired product could be detected by LCMS. The resulting mixture was quenched with water (150 mL). The resulting mixture was extracted with CHCl3/IPA (3:1, 3×150 mL). The combined organic layers were washed with brine (1×400 mL), and dried over anhydrous Na₂SO₄. After filtration, the filtrate was concentrated under reduced pressure. The residue was purified by reversed-phase flash chromatography with the following conditions: (column, C18; mobile phase, MeCN in Water (0.1% NH₃·H₂O+10 mmol/L NH₄HCO₃), 50% to 100% gradient in 20 min; detector, UV 254 nm) to afford 4-{[(1S,4S,5S)-2-{[(tert-butyldimethylsilyl)imino](methyl)oxo-lambda6-sulfanyl}-2-azabicyclo[2.2.1]heptan-5-yl]methyl}-6-isopropyl-2-(trifluoromethoxy) pyrazolo[1,5-a]pyrimidin-7-one.

ESI-MS m/z=562.20 [M+H]⁺; Calculated: 561.2.

### Step 8: 6-Isopropyl-4-(((1S,4S,5S)-2-(S-methylsulfonimidoyl)-2-azabicyclo[2.2.1]heptan-5-yl)methyl)-2-(trifluoromethoxy) pyrazolo[1,5-a]pyrimidin-7(4H)-one

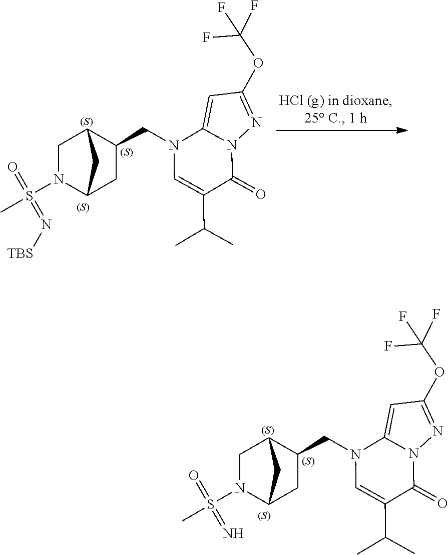

A solution of 4-{[(1S,4S,5S)-2-{[(tert-butyldimethylsilyl)imino](methyl)oxo-lambda6-sulfanyl}-2-azabicyclo[2.2.1]heptan-5-yl]methyl}-6-isopropyl-2-(trifluoromethoxy) pyrazolo[1,5-a]pyrimidin-7-one (300 mg, 0.53 mmol, 1.0 equiv) in 4M HCl (gas) in 1,4-dioxane (3 mL) was stirred at 25° C. for 1 h. The desired product could be detected by LCMS. The resulting mixture was concentrated under reduced pressure. The crude product (200 mg) was purified by Prep-HPLC with the following conditions (Column: XBridge Shield RP18 OBD Column 30*150 mm, 5 μm; Mobile Phase A: Water (10 mmol/L NH₄HCO₃+0.05% NH₃·H₂O), Mobile Phase B: ACN; Flow rate: 60 mL/min; Gradient: 20% B to 36% B in 10 min; Wave Length: 254 nm/220 nm nm; RT1 (min): 9.5) the pure fraction was concentrated then lyophilized to afford 6-isopropyl-4-(((1S,4S,5S)-2-(S-methylsulfonimidoyl)-2-azabicyclo[2.2.1]heptan-5-yl)methyl)-2-(trifluoromethoxy) pyrazolo[1,5-a]pyrimidin-7(4H)-one.

ESI-MS m/z=448.10 [M+H]⁺; Calculated: 447.2.

¹H NMR (400 MHZ, DMSO-d₆) δ 7.89 (s, 1H), 6.54 (s, 1H), 4.07-3.95 (m, 2H), 3.95-3.84 (m, 1H), 3.51-3.41 (m, 1H), 3.20-3.06 (m, 1H), 3.05-2.93 (m, 1H), 2.84-2.67 (m, 4H), 2.30 (q, J=4.2 Hz, 2H), 1.90-1.72 (m, 2H), 1.52 (d, J=10.3 Hz, 1H), 1.32-1.23 (m, 1H), 1.20 (d, J=6.9 Hz, 6H).

¹⁹F NMR (377 MHZ, DMSO) δ −57.62.

Total proton count from structure: 24.

Total proton count from spectrum: 24.

### Compound 47: Rel-(S)-2-cyclopropyl-6-isopropyl-3-methyl-4-(4-(S-methylsulfonimidoyl)benzyl)pyrazolo[1,5-a]pyrimidin-7(4H)-one

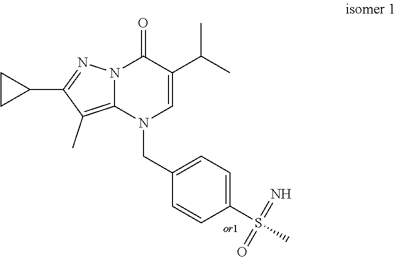 [SMILES: Cc1c(C2CC2)nn2c(=O)c(C(C)C)cn(Cc3ccc([S@@](C)(=N)=O)cc3)c12]

### Step 1: Rel-(S)-2-cyclopropyl-6-isopropyl-3-methyl-4-(4-(S-methylsulfonimidoyl)benzyl)pyrazolo[1,5-a]pyrimidin-7(4H)-one

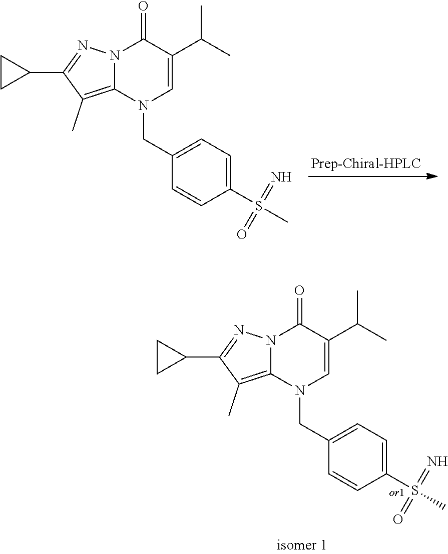 [SMILES: Cc1c(C2CC2)nn2c(=O)c(C(C)C)cn(Cc3ccc(S(C)(=N)=O)cc3)c12 ; Cc1c(C2CC2)nn2c(=O)c(C(C)C)cn(Cc3ccc([S@@](C)(=N)=O)cc3)c12]

The racemate (100 mg) was purified by Prep-Chiral-HPLC with the following conditions (Column: CHIRAL ART Cellulose-SZ, 3*25 cm, 5 μm; Mobile Phase A: Hex (10 mM NH₃-MeOH), Mobile Phase B: ETOH; Flow rate: 40 mL/min; Gradient: isocratic 50; Wave Length: 202/224 nm; RT1 (min): 15.4; Sample Solvent: ETOH: MEOH: DCM=1:1:1; Number Of Runs: 5), the pure factions were concentrated under vacuum then lyophilized to afford rel-(S)-2-cyclopropyl-6-isopropyl-3-methyl-4-(4-(S-methylsulfonimidoyl)benzyl)pyrazolo[1,5-a]pyrimidin-7(4H)-one.

ESI-MS m/z=399.10 [M+H]⁺; Calculated MW: 398.18.

¹H NMR (400 MHZ, DMSO-d₆) δ 7.96-7.88 (m, 2H), 7.79 (br, 1H), 7.30 (m, 2H), 5.58 (s, 2H), 4.22 (s, 1H), 3.04 (d, J=1.1 Hz, 3H), 3.01-2.97 (m, 1H), 2.00 (s, 3H), 1.83-1.86 (m, 1H), 1.18 (d, J=6.9 Hz, 6H), 0.90-0.88 (m, 2H), 0.86-0.82 (m, 2H).

Total proton count from structure: 26.

Total proton count from spectrum: 26.

### Compound 48: 2-Cyclopropyl-4-((1-imino-1-oxido-2,3-dihydro-1H-1l4-benzo[b]thiophen-5-yl)methyl)-6-isopropyl-[1,2,4]triazolo[1,5-a]pyrimidin-7(4H)-one

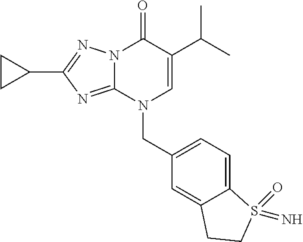 [SMILES: CC(C)c1cn(Cc2ccc3c(c2)CCS3(=N)=O)c2nc(C3CC3)nn2c1=O]

### Step 1: 2,3-Bihydrobenzo[b]thiophene 1,1-dioxide

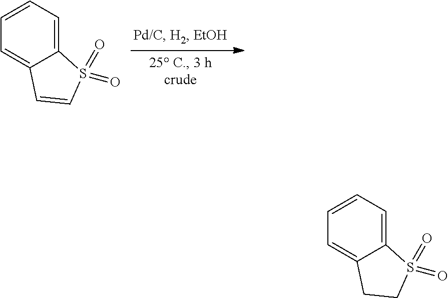 [SMILES: O=S1(=O)C=Cc2ccccc21 ; O=S1(=O)CCc2ccccc21]

To a stirred mixture of 1lambda6-benzothiophene-1,1-dione (10.00 g, 60.2 mmol, 1.0 equiv) in EtOH (100.00 mL) were added Pd/C (640 mg, 6.01 mmol, 0.1 equiv) at 25° C. under hydrogen atmosphere. The resulting mixture was stirred for 3 h at 25° C. under hydrogen atmosphere. The reaction was monitored by LCMS. The resulting mixture was filtered; the filter cake was washed with MeOH (200 mL). The filtrate was concentrated under reduced pressure. This resulted in 2,3-dihydro-1lambda6-benzothiophene-1,1-dione (crude).

ESI-MS m/z=168.9 [M+H]⁺; Calculated: 168.0.

### Step 2: 2,3-Bihydrobenzo[b]thiophene

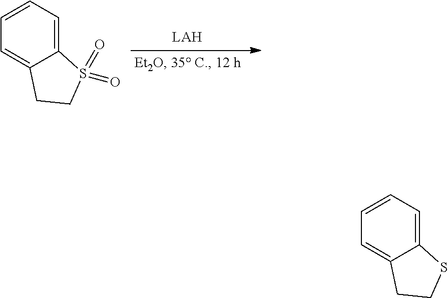 [SMILES: O=S1(=O)CCc2ccccc21 ; c1ccc2c(c1)CCS2]

To a stirred mixture of 2,3-dihydro-1lambda6-benzothiophene-1,1-dione (8.00 g, 47.6 mmol, 1.0 equiv) in diethyl ether (200.00 mL) were added LAH (214 mL, 428.0 mmol, 9.0 equiv, 2 M in THF) at 25° C. under nitrogen atmosphere. The resulting mixture was stirred for 12 h at 35° C. under nitrogen atmosphere. The mixture was allowed to cool down to 25° C. The reaction was monitored by TLC (PE:EA=5:1, Rf=0.2). The reaction was quenched with water at 0° C. The resulting mixture was diluted with 4N HCl (2 L). The resulting mixture was extracted with diethyl ether (3×1 L). The combined organic layers were washed with water (1×200 mL), and dried over anhydrous Na₂SO₄. After filtration, the filtrate was concentrated under reduced pressure. The residue was purified by silica gel column chromatography, eluted with PE/EA (5:1-3:1). This resulted in 2,3-dihydro-1-benzothiophene.

¹H NMR (400 MHZ, Chloroform-d) δ 7.14-7.07 (m, 2H), 7.02 (td, J=7.5, 1.3 Hz, 1H), 6.91 (td, J=7.4, 1.2 Hz, 1H), 3.29-3.23 (m, 2H), 3.18 (dd, J=8.5, 5.3 Hz, 2H).

### Step 3:5-Bromo-2,3-dihydrobenzo[b]thiophene

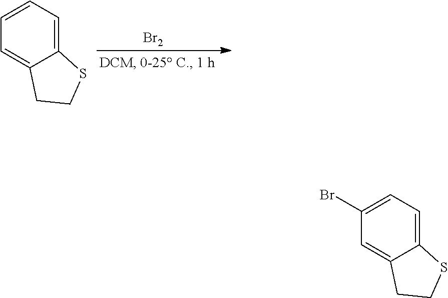 [SMILES: c1ccc2c(c1)CCS2 ; "Br2" ; Brc1ccc2c(c1)CCS2]

To a stirred mixture of 2,3-dihydro-1-benzothiophene (7.00 g, 51.4 mmol, 1.0 equiv) in DCM (70.00 mL) were added Br₂ (4.10 g, 25.7 mmol, 0.5 equiv) at 0° C. under nitrogen atmosphere. The resulting mixture was stirred for 1 h at 25° C. under nitrogen atmosphere. The reaction was monitored by LCMS. The reaction was quenched with sat. NaHCO₃(aq.) at 0° C. The resulting mixture was extracted with CH₂Cl₂ (3×100 mL), and dried over anhydrous Na₂SO₄. After filtration, the filtrate was concentrated under reduced pressure. The residue was purified by silica gel column chromatography, eluted with PE/EA (3:1-1:1) to afford 5-bromo-2,3-dihydro-1-benzothiophene. 1H NMR (400 MHZ, DMSO-d₆) δ 7.40 (dt, J=2.1, 1.1 Hz, 1H), 7.29-7.24 (m, 1H), 7.17 (d, J=8.2 Hz, 1H), 3.39-3.34 (m, 2H), 3.27-3.21 (m, 2H).

### Step 4: (2,3-Dihydrobenzo[b]thiophen-5-yl)metanol

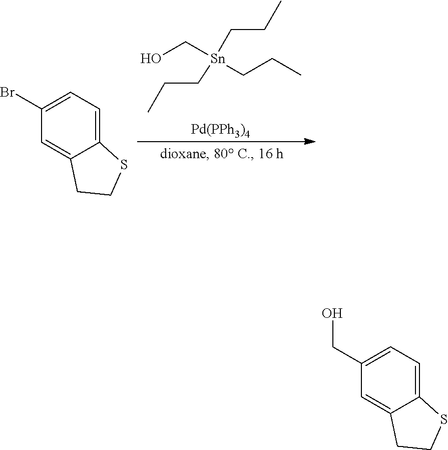

To a solution of 2,3-dihydro-1-benzothiophen-5-ylmethanol (4.00 g, 24.1 mmol, 1.0 equiv) in dioxane (40.00 mL) were added (Tributylstannyl)methanol (11.59 g, 36.1 mmol, 1.5 equiv) and Pd(PPh₃)₄ (2.78 g, 2.41 mmol, 0.10 equiv) at 25° C. under nitrogen atmosphere. The resulting mixture was stirred for 16 h at 80° C. under nitrogen atmosphere. The reaction was monitored by TLC (EA: PE=1:10 Rf=0.6). The mixture was allowed to cool down to 25° C. The resulting mixture was diluted with water (20.00 mL). The resulting mixture was extracted with EtOAc (3×30 mL), and dried over anhydrous Na₂SO₄. After filtration, the filtrate was concentrated under reduced pressure. The residue was purified by silica gel column chromatography, eluted with PE/EA (3:1-1:1) to afford 2,3-dihydro-1-benzothiophen-5-ylmethanol.

¹H NMR (400 MHZ, DMSO-d₆) δ 7.26-7.19 (m, 2H), 7.13-7.09 (m, 1H), 5.19 (td, J=5.7, 2.9 Hz, 1H), 4.47 (d, J=5.3 Hz, 2H), 3.40 (dd, J=8.6, 7.2 Hz, 2H), 3.27 (t, J=7.8 Hz, 2H).

### Step 5: 5-(Hydroxymethyl)-1-imino-2,3-dihydro-1H-1l4-benzo[b]thiophene 1-oxide

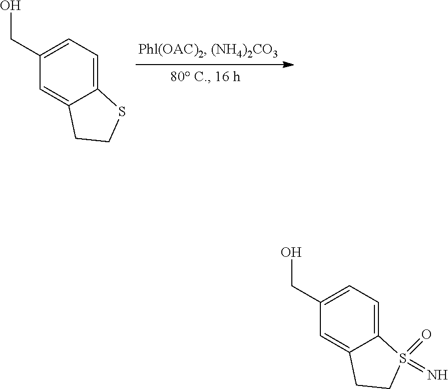 [SMILES: OCc1ccc2c(c1)CCS2 ; N=S1(=O)CCc2cc(CO)ccc21]

To a solution of 2,3-dihydro-1-benzothiophen-5-ylmethanol (800 mg, 4.81 mmol, 1.0 equiv) and (diacetoxyiodo)benzene (7.75 g, 24.1 mmol, 5.0 equiv) in MeOH (16.00 mL) was added (NH₄)₂CO₃ (1.39 g, 14.5 mmol, 3.0 equiv) at 0° C. under nitrogen atmosphere. The resulting mixture was stirred for 16 h at 80° C. under nitrogen atmosphere. The reaction was monitored by LCMS. The mixture was allowed to cool down to 25° C. The resulting mixture was concentrated under reduced pressure. The residue was purified by silica gel column chromatography, eluted with CH₂Cl₂/MeOH (10:1-5:1) to afford 5-(hydroxymethyl)-1-imino-2,3-dihydro-1lambda6-benzothiophen-1-one.

ESI-MS m/z=198.1 [M+H]⁺; Calculated: 197.1.

### Step 6: 5-(Bromomethyl)-1-imino-2,3-dihydro-1H-1l4-benzo[b]thiophene 1-oxide

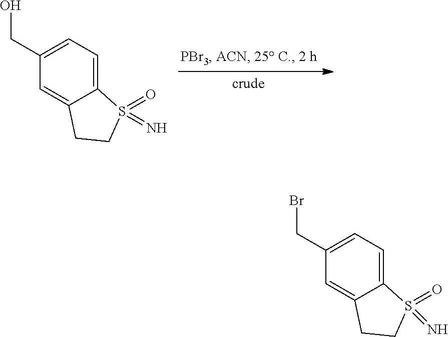 [SMILES: N=S1(=O)CCc2cc(CO)ccc21 ; N=S1(=O)CCc2cc(CBr)ccc21]

To a solution of 5-(hydroxymethyl)-1-imino-2,3-dihydro-1lambda6-benzothiophen-1-one (480 mg, 2.43 mmol, 0.1 equiv) in ACN (6.00 mL) was added PBr₃ (659 mg, 2.43 mmol, 1.0 equiv) at 25° C. under nitrogen atmosphere. The resulting mixture was stirred for 2 h at 25° C. under nitrogen atmosphere. The reaction was monitored by LCMS. The resulting mixture was concentrated under reduced pressure. The residue was purified by trituration with ethyl ether (5.00 mL) to afford 5-(bromomethyl)-1-imino-2,3-dihydro-1lambda6-benzothiophen-1-one (crude).

ESI-MS m/z=259.9, 261.9 [M+H]⁺; Calculated: 259.0, 261.0.

### Step 7: 2-Cyclopropyl-4-((1-imino-1-oxido-2,3-dihydro-1H-1l4-benzo[b]thiophen-5-yl)methyl)-6-isopropyl-[1,2,4]triazolo[1,5-a]pyrimidin-7(4H)-one

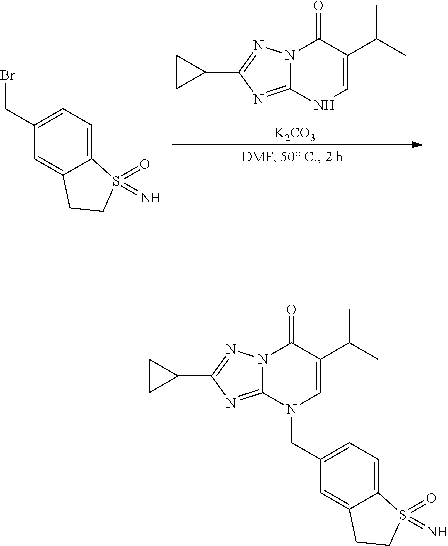

To a solution of 5-(bromomethyl)-1-imino-2,3-dihydro-1lambda6-benzothiophen-1-one (500 mg, 1.92 mmol, 1.0 equiv) and 2-cyclopropyl-6-isopropyl-4H-[1,2,4]triazolo[1,5-a]pyrimidin-7-one (503 mg, 2.31 mmol, 1.2 equiv) in DMF (5.00 mL) was added K₂CO₃ (797 mg, 5.77 mmol, 3.0 equiv) at 25° C. under nitrogen atmosphere. The resulting mixture was stirred for 2 h at 50° C. under nitrogen atmosphere. The reaction was monitored by LCMS. The mixture was allowed to cool down to 25° C. The resulting mixture was diluted with water (50 mL). The resulting mixture was extracted with CHCl₃:IPA=3:1 (3×50 mL), and dried over anhydrous Na₂SO₄. After filtration, the filtrate was concentrated under reduced pressure. The crude product was purified by Prep-HPLC with the following conditions (Column: XBridge Prep Shield RP18 5 μm 19*10 mm; Mobile Phase A: 10 mmol/L NH₄HCO₃+0.05% NH₃H₂O), Mobile Phase B: ACN; Flow rate: 60 mL/min mL/min; Gradient: 5% B to 5% B in 1 min, 5% B to 13% B in 2 min, 13% to 30% B in 10 min; Wave Length: 254 nm/220 nm nm; RT1 (min): 9.3), the pure factions were concentrated under vacuum then lyophilized to afford 2-cyclopropyl-4-((1-imino-1-oxido-2,3-dihydro-1H-1l4-benzo[b]thiophen-5-yl)methyl)-6-isopropyl-[1,2,4]triazolo[1,5-a]pyrimidin-7(4H)-one.

ESI-MS m/z=398.10 [M+H]⁺; Calculated: 397.2.

¹H NMR (400 MHZ, DMSO-d₆) δ 8.02-8.00 (m, 1H), 7.62 (d, J=8.0 Hz, 1H), 7.47-7.40 (m, 2H), 5.37 (s, 2H), 4.51 (s, 1H), 3.44 (t, J=6.8 Hz, 2H), 3.24 (td, J=6.8, 2.5 Hz, 2H), 3.03-2.95 (m, 1H), 2.04 (tt, J=8.3, 4.9 Hz, 1H), 1.19 (d, J=6.9 Hz, 6H), 1.04-0.97 (m, 2H), 0.90 (dt, J=4.7, 3.0 Hz, 2H).

Total proton count from structure: 23.

Total proton count from spectrum: 23.

### Compound 49: Rel-(S)-2-cyclopropyl-6-isopropyl-4-(4-(S-(trifluoromethyl) sulfonimidoyl)benzyl)-[1,2,4]triazolo[1,5-a]pyrimidin-7(4H)-one

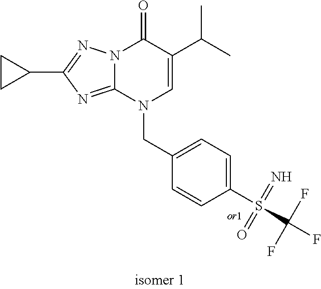 [SMILES: CC(C)c1cn(Cc2ccc([S@](=N)(=O)C(F)(F)F)cc2)c2nc(C3CC3)nn2c1=O]

### Step 1: (4-((Trifluoromethyl)thio)phenyl)methanol

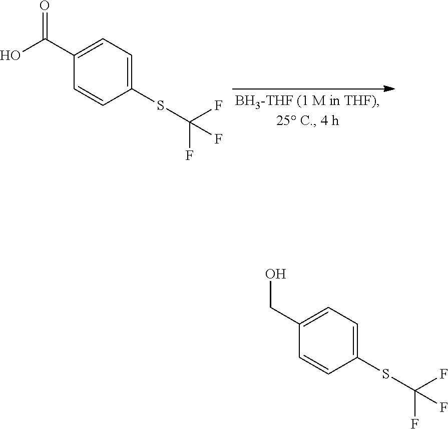 [SMILES: O=C(O)c1ccc(SC(F)(F)F)cc1 ; OCc1ccc(SC(F)(F)F)cc1]

A solution of 4-[(trifluoromethyl)sulfanyl]benzoic acid (5.00 g, 22.5 mmol, 1.0 equiv) in Borane-tetrahydrofuran complex (1.0M in THF) (50 mL) was stirred at 25° C. for 4 h under nitrogen atmosphere. The desired product could be detected by LCMS. The reaction was quenched with water at 0° C. The resulting mixture was extracted with EtOAc (3×50 mL). The combined organic layers were washed with brine (2×10 mL), and dried over anhydrous Na₂SO₄. After filtration, the filtrate was concentrated under reduced pressure. The residue was purified by silica gel column chromatography, eluted with PE/EA (3:1) to afford (4-((trifluoromethyl)thio)phenyl)methanol.

ESI-MS m/z=206.9 [M−H]; Calculated: 208.0.

¹H NMR (400 MHZ, DMSO-d₆) δ7.67 (d, J=7.8 Hz, 2H), 7.48 (d, J=8.0 Hz, 2H), 5.37 (td, J=5.8, 2.1 Hz, 1H), 4.57 (d, J=5.7 Hz, 2H).

¹⁹F NMR (376 MHZ, DMSO) δ −42.41

### Step 2: (4-(Bromomethyl)phenyl) (trifluoromethyl) sulfane

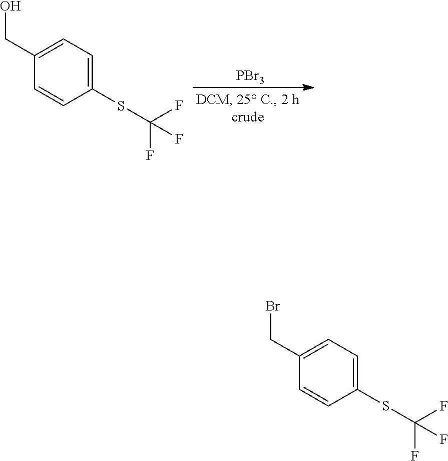 [SMILES: OCc1ccc(SC(F)(F)F)cc1 ; "PBr3" ; FC(F)(F)Sc1ccc(CBr)cc1]

To a stirred solution of (4-((trifluoromethyl)thio)phenyl)methanol (2.00 g, 9.60 mmol, 1.0 equiv) in DCM (20 mL) was added PBr₃ (7.80 g, 28.8 mmol, 3.0 equiv) dropwise at 0° C. under nitrogen atmosphere. The resulting mixture was stirred at 25° C. for 2 h under nitrogen atmosphere. The desired product could be detected by LCMS. The resulting mixture was concentrated under reduced pressure. The residue was purified by trituration with ethyl ether (3×10 mL). This resulted in (4-(bromomethyl)phenyl) (trifluoromethyl) sulfane (crude).

ESI-MS m/z=269.0&271.0 [M−H]⁻; Calculated: 269.9&271.9.

### Step 3: 2-Cyclopropyl-6-isopropyl-4-(4-((trifluoromethyl)thio)benzyl)-[1,2,4]triazolo[1,5-a]pyrimidin-7(4H)-one

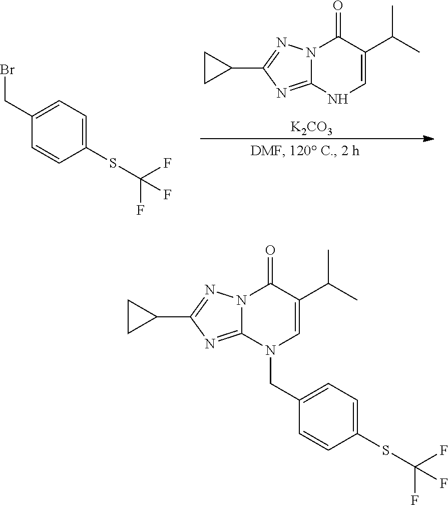

To a stirred solution of (4-(bromomethyl)phenyl) (trifluoromethyl) sulfane (1.50 g, 5.53 mmol, 1.0 equiv) and 2-cyclopropyl-6-isopropyl-4H-[1,2,4]triazolo[1,5-a]pyrimidin-7-one (1.21 g, 5.53 mmol, 1.0 equiv) in DMF (15 mL) was added K₂CO₃ (2.29 g, 16.5 mmol, 3.0 equiv) in portions at 25° C. under nitrogen atmosphere. The resulting mixture was stirred at 120° C. for 2 h under nitrogen atmosphere. The desired product could be detected by LCMS. The mixture was allowed to cool down to 25° C. The resulting mixture was diluted with EtOAc (100 mL). The resulting mixture was washed with 5×50 ml of water. The combined organic layers were washed with brine (2×30 mL), and dried over anhydrous Na₂SO₄. After filtration, the filtrate was concentrated under reduced pressure. The residue was purified by silica gel column chromatography, eluted with PE/EA (1:1) to afford 2-cyclopropyl-6-isopropyl-4-(4-((trifluoromethyl)thio)benzyl)-[1,2,4]triazolo[1,5-a]pyrimidin-7(4H)-one.

ESI-MS m/z=409.1 [M+H]⁺; Calculated: 408.1.

¹H NMR (400 MHZ, DMSO-d₆) δ8.03 (s, 1H), 7.72 (d, J=8.0 Hz, 2H), 7.53-7.47 (m, 2H), 5.39 (s, 2H), 2.99 (hept, J=6.9 Hz, 1H), 2.04 (ddt, J=13.1, 8.5, 4.8 Hz, 1H), 1.19 (d, J=6.9 Hz, 6H), 0.99 (dt, J=8.3, 3.2 Hz, 2H), 0.88 (dt, J=5.2, 3.1 Hz, 2H).

¹⁹F NMR (376 MHZ, DMSO) δ −41.89.

### Step 4: 2-Cyclopropyl-6-isopropyl-4-(4-(S-(trifluoromethyl) sulfonimidoyl)benzyl)-[1,2,4]triazolo[1,5-a]pyrimidin-7(4H)-one

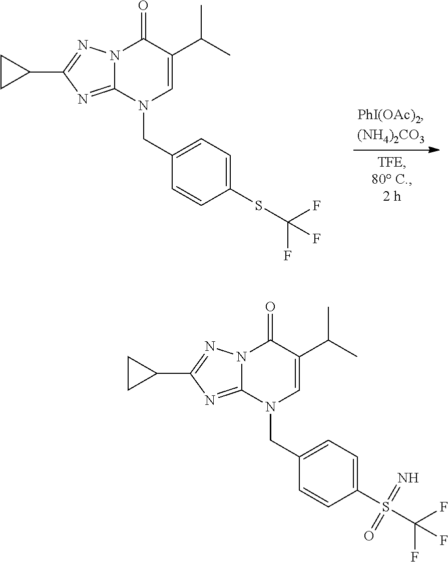 [SMILES: CC(C)c1cn(Cc2ccc(SC(F)(F)F)cc2)c2nc(C3CC3)nn2c1=O ; CC(C)c1cn(Cc2ccc(S(=N)(=O)C(F)(F)F)cc2)c2nc(C3CC3)nn2c1=O]

To a stirred solution of 2-cyclopropyl-6-isopropyl-4-(4-((trifluoromethyl)thio) benzyl)-[1,2,4]triazolo[1,5-a]pyrimidin-7(4H)-one (900 mg, 2.20 mmol, 1.0 equiv) and (diacetoxyiodo)benzene (1.63 g, 5.06 mmol, 2.3 equiv) in 2,2,2-trifluoroethan-1-ol (10 mL) was added ammonium carbonate (317 mg, 3.30 mmol, 1.5 equiv) in portions at 25° C. under air atmosphere. The resulting mixture was stirred at 80° C. for 2 h under air atmosphere. The desired product could be detected by LCMS. The mixture was allowed to cool down to 20° C. The resulting mixture was diluted with EtOAc (30 mL). The resulting mixture was washed with 2×20 ml of water. The resulting mixture was extracted with EtOAc (3×30 mL). The combined organic layers were washed with brine (2×10 mL), and dried over anhydrous Na₂SO₄. After filtration, the filtrate was concentrated under reduced pressure. The residue was purified by silica gel column chromatography, eluted with PE/EA (1:1) to afford 2-cyclopropyl-6-isopropyl-4-(4-(S-(trifluoromethyl) sulfonimidoyl)benzyl)-[1,2,4]triazolo[1,5-a]pyrimidin-7(4H)-one (crude). The crude was purified by Prep-HPLC with the following conditions: (Column: XBridge Prep Phenyl OBD Column 19*250 mm, 5 m; Mobile Phase A: Water (10 mmol/L NH₄HCO₃), Mobile Phase B: ACN; Flow rate: 60 mL/min mL/min; Gradient: 5% B to 5% B in 1 min, 5% B to 34% B in 2 min, 34% to 54% B in 11 min; Wave Length: 254 nm/220 nm nm; RT1 (min): 9.07) to afford 2-cyclopropyl-6-isopropyl-4-(4-(S-(trifluoromethyl) sulfonimidoyl)benzyl)-[1,2,4]triazolo[1,5-a]pyrimidin-7(4H)-one.

ESI-MS m/z=440.1 [M+H]⁺; Calculated: 439.1.

¹H NMR (400 MHZ, DMSO-d₆) δ 8.06 (d, J=8.3 Hz, 2H), 8.04-8.01 (m, 1H), 7.71-7.63 (m, 2H), 6.86 (s, 1H), 5.48 (s, 2H), 2.99 (hept, J=6.9 Hz, 1H), 2.08-1.99 (m, 1H), 1.19 (d, J=6.9 Hz, 6H), 1.01-0.95 (m, 2H), 0.88-0.83 (m, 2H).

¹⁹F NMR (376 MHZ, DMSO) δ −78.08.

### Step 5: Rel-(S)-2-cyclopropyl-6-isopropyl-4-(4-(S-(trifluoromethyl) sulfonimidoyl)benzyl)-[1,2,4]triazolo[1,5-a]pyrimidin-7(4H)-one

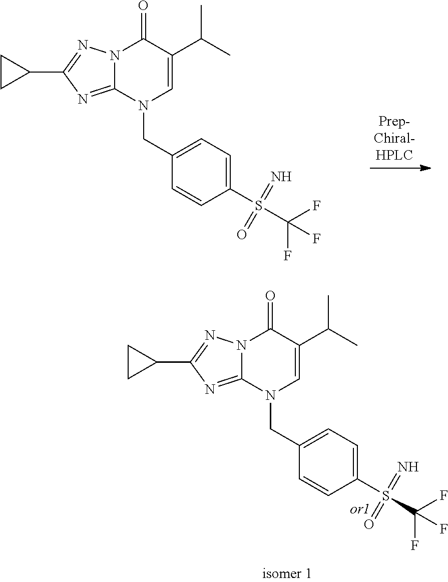

The racemate (350 mg) was separated by Prep-Chiral-HPLC with the following conditions: (Column: Chiral NQ(2)5 u, 250*30 mm; Mobile Phase A: Hex (10 mM NH₃-MeOH), Mobile Phase B: EtOH; Flow rate: 40 mL/min; Gradient: isocratic 40; Wave Length: 222/286 nm; RT1 (min): 7.3; RT2 (min): 8.8; Sample Solvent: EtOH: MeOH=1:3-HPLC; Number Of Runs: 25), the pure fraction was concentrated then lyophilized to afford rel-(S)-2-cyclopropyl-6-isopropyl-4-(4-(S-(trifluoromethyl) sulfonimidoyl)benzyl)-[1,2,4]triazolo[1,5-a]pyrimidin-7(4H)-one.

ESI-MS m/z=440.15 [M+H]⁺; Calculated: 439.1.

¹H NMR (400 MHZ, DMSO-d₆) δ 8.06 (d, J=8.2 Hz, 2H), 8.03 (s, 1H), 7.68 (d, J=8.2 Hz, 2H), 6.87 (s, 1H), 5.49 (s, 2H), 3.00 (h, J=6.9 Hz, 1H), 2.03 (tt, J=8.5, 4.8 Hz, 1H), 1.19 (d, J=6.9 Hz, 6H), 1.01-0.95 (m, 2H), 0.88-0.83 (m, 2H).

¹⁹F NMR (376 MHZ, DMSO) δ −78.18.

Total proton count from structure: 20.

Total proton count from spectrum: 20.

### Compound 50: Rel-(S)-2-cyclopropyl-4-(4-(S-(difluoromethyl) sulfonimidoyl)benzyl)-6-isopropyl-[1,2,4]triazolo[1,5-a]pyrimidin-7(4H)-one

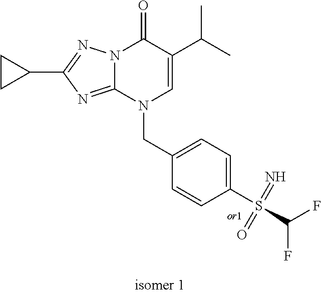 [SMILES: CC(C)c1cn(Cc2ccc([S@](=N)(=O)C(F)F)cc2)c2nc(C3CC3)nn2c1=O]

### Step 1: 4-[(Difluoromethyl)sulfanyl]benzoic acid

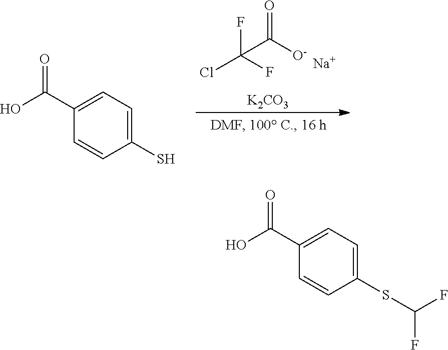

A solution of 2-mercaptobenzoic acid (10.00 g, 64.8 mmol, 1.0 equiv), sodium 2-chloro-2,2-difluoroacetate (14.83 g, 97.2 mmol, 1.5 equiv) and K₂CO₃ (26.89 g, 194.5 mmol, 3.0 equiv) in DMF (100 mL) was stirred at 100° C. for 16 h under nitrogen atmosphere. The reaction was monitored by LCMS. The mixture was allowed to cool down to 25° C. The resulting mixture was diluted with water (200 mL). The mixture was basified to pH 3 with HCl (aq.). The resulting mixture was extracted with EtOAc (3×100 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (1:1) to afford 4-[(difluoromethyl)sulfanyl]benzoic acid.

ESI-MS m/z=202.85 [M−H]⁻; Calculated: 204.1.

¹H NMR (400 MHZ, DMSO-d₆) δ 13.30-12.94 (m, 1H), 8.16-7.40 (m, 5H).

¹⁹F NMR (376 MHZ, DMSO) δ −92.60.

### Step 2: {4-[(Difluoromethyl) sulfanyl]phenyl}methanol

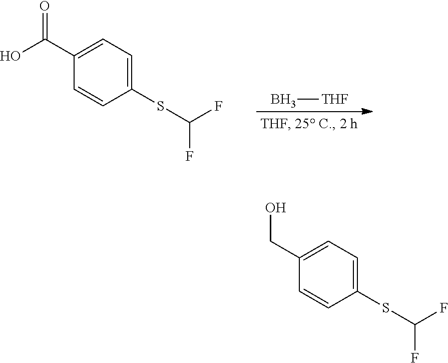

To a mixture of 4-[(difluoromethyl)sulfanyl]benzoic acid (3.50 g, 17.1 mmol, 1.0 equiv) in THF (15 mL) was added Borane-tetrahydrofuran complex (1.0 M in THF) (15 mL) at 25° C. under nitrogen atmosphere. The resulting mixture was stirred for 2 h at 25° C. The reaction was monitored by LCMS. The reaction was quenched with Water at 0° C. The mixture was extracted with EtOAc (3×100 mL). The resulting mixture was concentrated under reduced pressure. The residue was purified by reversed-phase flash chromatography with the following conditions: column, C18; mobile phase, MeCN in Water (0.1% FA), 10% to 50% gradient in 20 min; detector, UV 254 nm. This resulted in {4-[(difluoromethyl) sulfanyl]phenyl}methanol.

ESI-MS m/z=173.00 [M+H]⁺; Calculated: 173.2.

¹H NMR (400 MHZ, DMSO-d6) δ 7.62-7.24 (m, 5H), 5.35 (d, J=4.4 Hz, 1H), 4.62-4.48 (m, 2H).

¹⁹F NMR (376 MHZ, DMSO) δ 92.69.

### Step 3: (Difluoromethyl) [4-(hydroxymethyl)phenyl]imino-lambda6-sulfanone

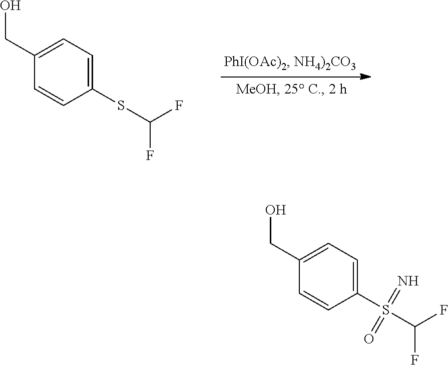 [SMILES: OCc1ccc(SC(F)F)cc1 ; N=S(=O)(c1ccc(CO)cc1)C(F)F]

To a mixture of {4-[(difluoromethyl) sulfanyl]phenyl}methanol (1.60 g, 8.41 mmol, 1.0 equiv) and (diacetoxyiodo)benzene (6.23 g, 19.3 mmol, 2.3 equiv) in methanol (20.00 mL) was added ammonium carbonate (1.21 g, 12.6 mmol, 1.5 equiv) at 25° C. under nitrogen atmosphere. The resulting mixture was stirred for 2 h at 25° C. The resulting mixture was concentrated under reduced pressure. The residue was purified by reversed-phase flash chromatography with the following conditions: column, C18; mobile phase, MeCN in Water (0.1% FA), 10% to 50% gradient in 30 min; detector, UV 254 nm. This resulted in (difluoromethyl) [4-(hydroxymethyl)phenyl]imino-lambda6-sulfanone.

ESI-MS m/z=221.95 [M+H]⁺; Calculated: 221.2.

¹H NMR (400 MHZ, DMSO-d₆) δ 7.99-7.88 (m, 2H), 7.63 (d, J=8.1 Hz, 2H), 6.86 (t, J=53.6 Hz, 1H), 5.60-5.52 (m, 2H), 4.65 (s, 2H).

¹⁹F NMR (376 MHZ, DMSO) δ −120.47, −121.15, −122.32, −123.00.

### Step 4: [4-(Bromomethyl)phenyl](difluoromethyl)imino-lambda6-sulfanone

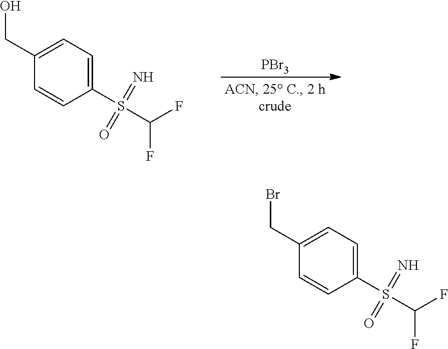 [SMILES: N=S(=O)(c1ccc(CO)cc1)C(F)F ; "PBr3" ; N=S(=O)(c1ccc(CBr)cc1)C(F)F]

To a mixture of (difluoromethyl) [4-(hydroxymethyl)phenyl]imino-lambda6-sulfanone (500 mg, 2.26 mmol, 1.0 equiv) in acetonitrile (6 mL) was added PBr₃ (1.84 g, 6.78 mmol, 3.0 equiv) at 0° C. under nitrogen atmosphere. The resulting mixture was stirred for 2 h at 25° C. The reaction was monitored by LCMS. The desired product could be detected by LCMS. The resulting mixture was concentrated under reduced pressure. The residue was purified by trituration with diethyl ether (200 mL). This resulted in [4-(bromomethyl)phenyl](difluoromethyl)imino-lambda6-sulfanone.

ESI-MS m/z=283.85, 285.85 [M+H]⁺; Calculated: 282.9, 284.9.

### Step 5: 2-Cyclopropyl-4-({4-[(difluoromethyl)(imino)oxo-lambda6-sulfanyl]phenyl}methyl)-6-isopropyl-[1,2,4]triazolo[1,5-a]pyrimidin-7-one

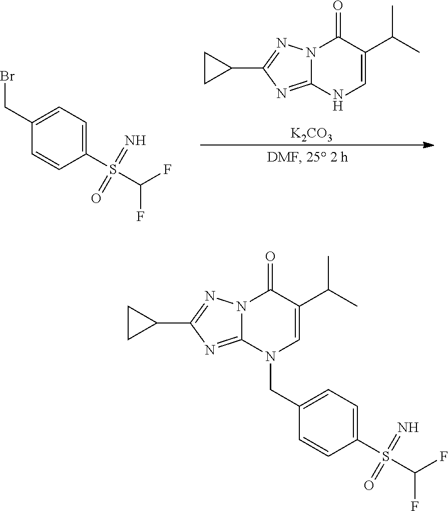

To a mixture of [4-(bromomethyl)phenyl](difluoromethyl)imino-lambda6-sulfanone (1.00 g, 3.52 mmol, 1.0 equiv) and 2-cyclopropyl-6-isopropyl-4H-[1,2,4]triazolo[1,5-a]pyrimidin-7-one (0.54 g, 2.46 mmol, 0.7 equiv) in DMF (5 mL) was added K₂CO₃ (1.46 g, 10.5 mmol, 3.0 equiv) at 25° C. under nitrogen atmosphere. The resulting mixture was stirred for 2 h at 25° C. The desired products could be detected by LCMS. The resulting mixture was diluted with water (50 mL). The aqueous layer was extracted with EtOAc (3×50 mL). The resulting mixture was concentrated under reduced pressure. The crude product (800 mg) was purified by Prep-HPLC with the following conditions ((Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 16% B to 31% B in 8 min; Wave Length: 254 nm/220 nm nm; RT1 (min): 9.33) to afford 2-cyclopropyl-4-({4-[(difluoromethyl)(imino)oxo-lambda6-sulfanyl]phenyl}methyl)-6-isopropyl-[1,2,4]triazolo[1,5-a]pyrimidin-7-one.

ESI-MS m/z=422.15 [M+H]⁺; Calculated: 421.5.

¹H NMR (400 MHZ, DMSO-d₆) δ 8.04 (s, 1H), 7.95 (d, J=8.4 Hz, 2H), 7.63 (d, J=8.4 Hz, 2H), 6.88 (t, J=53.5 Hz, 1H), 5.62 (s, 1H), 5.47 (s, 2H), 2.99 (p, J=6.9 Hz, 1H), 2.03 (ddd, J=13.1, 8.4, 4.9 Hz, 1H), 1.19 (d, J=6.9 Hz, 6H), 1.03-0.95 (m, 2H), 0.87 (dd, J=4.8, 2.5 Hz, 2H).

¹⁹F NMR (376 MHZ, DMSO) δ −120.52, −121.21, −122.32, −123.01.

### Step 6: Rel-(S)-2-cyclopropyl-4-(4-(S-(difluoromethyl) sulfonimidoyl)benzyl)-6-isopropyl-[1,2,4]triazolo[1,5-a]pyrimidin-7(4H)-one

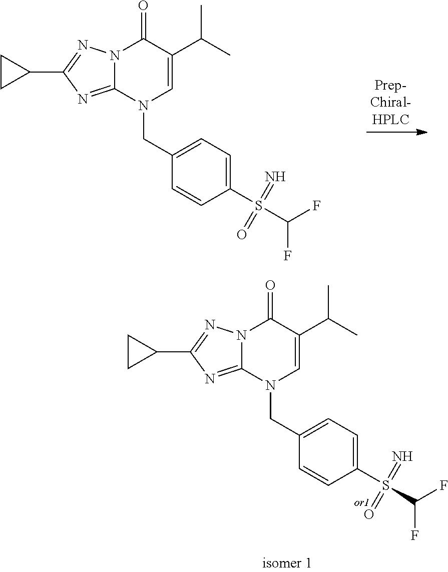

The racemate (200 mg) was purified by Prep-Chiral HPLC with the following conditions (Column: Chiral NQ(2)5 u, 250*30 mm; Mobile Phase A: Hex (10 mM NH₃-MeOH), Mobile Phase B: ETOH; Gradient: isocratic 45; Wave Length: 222/286 nm; RT1 (min): 10.5; RT2 (min): 11.9; Sample Solvent: Hex (10 mM NH₃-MeOH); Number Of Runs: 20), the pure factions were concentrated under vacuum then lyophilized to afford rel-(S)-2-cyclopropyl-4-(4-(S-(difluoromethyl) sulfonimidoyl)benzyl)-6-isopropyl-[1,2,4]triazolo[1,5-a]pyrimidin-7(4H)-one.

ESI-MS m/z=422.15 [M+H]⁺; Calculated: 421.5.

¹H NMR (400 MHZ, DMSO-d₆) δ 8.09-7.90 (m, 3H), 7.63 (d, J=8.1 Hz, 2H), 6.88 (t, J=53.6 Hz, 1H), 5.62 (s, 1H), 5.46 (s, 2H), 2.99 (p, J=6.9 Hz, 1H), 2.03 (qd, J=9.5, 8.5, 5.3 Hz, 1H), 1.19 (d, J=6.9 Hz, 6H), 1.02-0.96 (m, 2H), 0.96-0.82 (m, 2H).

¹⁹F NMR (376 MHZ, DMSO) δ −120.51, −121.20, −122.31, −123.00.

Total proton count from structure: 21.

Total proton count from spectrum: 21.

### Compound 51: Rel-(R)-2-cyclopropyl-6-isopropyl-4-(4-(S-(methyl-d₃) sulfonimidoyl)benzyl)-[1,2,4]triazolo[1,5-a]pyrimidin-7(4H)-one

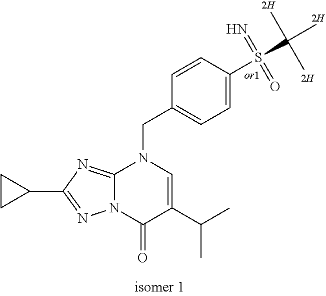

### Step 1: 4-[(2H3)methylsulfanyl]benzoic acid

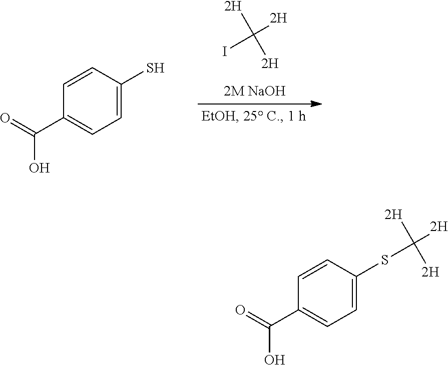

To a stirred mixture of 2-mercaptobenzoic acid (10.00 g, 64.8 mmol, 1.0 equiv) in EtOH (200 mL) were added iodomethane-d₃ (18.80 g, 129.7 mmol, 2.0 equiv) and NaOH (65 mL, 130.0 mmol, 2.0 equiv, 2M in water) dropwise at 25° C. under nitrogen atmosphere. The resulting mixture was stirred at 25° C. for 1 h under nitrogen atmosphere. The resulting mixture was diluted with water (1 L). The residue was acidified to pH 3 with 1M HCl (aq.). The precipitated solids were collected by filtration and washed with water (3×50 mL). The solids were concentrated under vacuum to afford 4-[(2H3)methylsulfanyl]benzoic acid.

ESI-MS m/z=212.95 [M+H+41]⁺; Calculated: 171.0.

¹H NMR (400 MHZ, DMSO-d₆) δ 12.83 (s, 1H), 7.85 (d, J=8.6 Hz, 2H), 7.33 (d, J=8.5 Hz, 2H).

### Step 2: {4-[(2H3)methylsulfanyl]phenyl}methanol

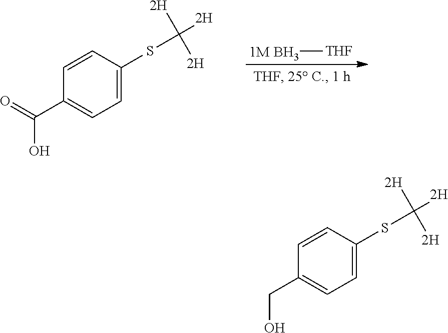

To a stirred solution of 4-[(2H3)methylsulfanyl]benzoic acid (11.00 g, 64.2 mmol, 1.0 equiv) in THF (130 mL) was added Borane-tetrahydrofuran complex (129 mL, 129.0 mmol, 2.0 equiv, 1.0 M in THF) dropwise at 10° C. under nitrogen atmosphere. The resulting mixture was stirred at 25° C. for 1 h under nitrogen atmosphere. The desired product could be detected by LCMS. The reaction was poured into water (750 mL) at 0° C. The resulting mixture was added brine (250 mL), then extracted with EtOAc (2×1 L). The combined organic layers were washed with brine (1×1 L), and dried over anhydrous Na₂SO₄. After filtration, the filtrate was concentrated under reduced pressure. The residue was purified by silica gel column chromatography, eluted with EA in PE (10% to 40% gradient in 20 min, hold at 27%) to afford {4-[(2H3)methylsulfanyl]phenyl}methanol.

ESI-MS m/z=156.15 [M−H]⁻; Calculated: 157.1.

¹H NMR (400 MHZ, DMSO-d₆) δ 7.30-7.18 (m, 4H), 5.48-4.64 (m, 1H), 4.44 (s, 2H).

### Step 3: 1-(Chloromethyl)-4-[(2H3)methylsulfanyl]benzene

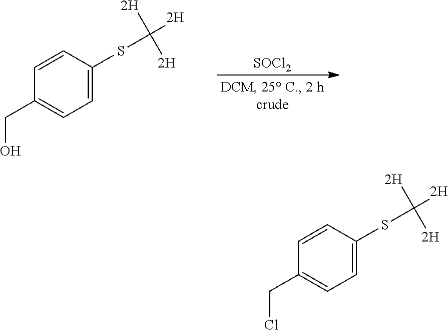

To a stirred mixture of {4-[(2H3)methylsulfanyl]phenyl}methanol (500 mg, 3.18 mmol, 1.0 equiv) in DCM (10 mL) was added Thionyl chloride (757 mg, 6.36 mmol, 2.0 equiv) dropwise at 25° C. under nitrogen atmosphere. The resulting mixture was stirred at 25° C. for 2 h under nitrogen atmosphere. The desired product could be detected by LCMS. The resulting mixture was concentrated under reduced pressure to afford 1-(chloromethyl)-4-[(2H3)methylsulfanyl]benzene (crude).

ESI-MS m/z=140.20 [M-Cl]⁺; Calculated: 175.0.

### Step 4: 2-Cyclopropyl-6-isopropyl-4-({4-[(2H3)methylsulfanyl]phenyl}methyl)-[1,2,4]triazolo[1,5-a]pyrimidin-7-one

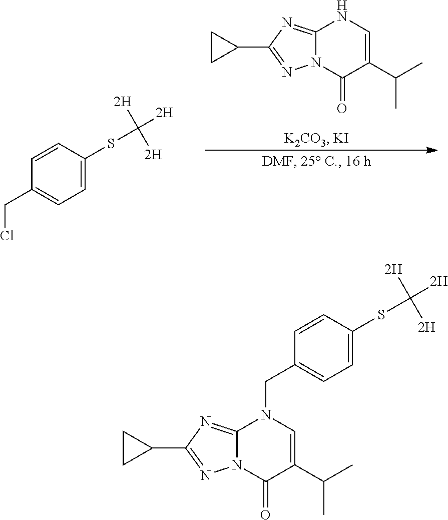

To a stirred mixture of 2-cyclopropyl-6-isopropyl-4H-[1,2,4]triazolo[1,5-a]pyrimidin-7-one (745 mg, 3.41 mmol, 1.0 equiv) and K₂CO₃ (1.42 g, 10.2 mmol, 3.0 equiv) in DMF (10 mL) were added KI (567 mg, 3.41 mmol, 1.0 equiv) and 1-(chloromethyl)-4-[(2H3)methylsulfanyl]benzene (600 mg, crude) at 25° C. under nitrogen atmosphere. The resulting mixture was stirred at 25° C. for 16 h under nitrogen atmosphere. The desired product could be detected by LCMS. The resulting mixture was diluted with water (100 mL). The resulting mixture was extracted with EtOAc (3×100 mL). The combined organic layers were washed with brine (1×200 mL), and dried over anhydrous Na₂SO₄. After filtration, the filtrate was concentrated under reduced pressure. The residue was purified by silica gel column chromatography, eluted with EA in PE (10% to 50% gradient in 30 min, hold at 43%) to afford 2-cyclopropyl-6-isopropyl-4-({4-[(2H3)methylsulfanyl]phenyl}methyl)-[1,2,4]triazolo[1,5-a]pyrimidin-7-one.

ESI-MS m/z=358.10 [M+H]⁺; Calculated: 357.2.

¹H NMR (400 MHZ, Methanol-d₄) δ 7.75 (d, J=0.8 Hz, 1H), 7.36-7.28 (m, 2H), 7.27-7.20 (m, 2H), 5.30 (s, 2H), 3.12-3.01 (m, 1H), 2.14-2.05 (m, 1H), 1.23 (d, J=6.9 Hz, 6H), 1.09-1.03 (m, 4H).

### Step 5: 2-Cyclopropyl-4-({4-[imino ((2H3)methyl) oxo-lambda6-sulfanyl]phenyl}methyl)-6-isopropyl-[1,2,4]triazolo[1,5-a]pyrimidin-7-one

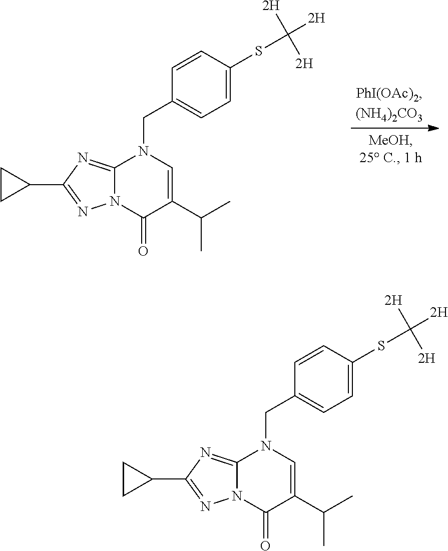

A mixture of 2-cyclopropyl-6-isopropyl-4-({4-[(2H3)methylsulfanyl]phenyl}methyl)-[1,2,4]triazolo[1,5-a]pyrimidin-7-one (500 mg, 1.40 mmol, 1.0 equiv) and (diacetoxyiodo)benzene (1.58 g, 4.90 mmol, 3.5 equiv) in methan-d₃-ol-d (10 mL) was stirred at 25° C. for 10 min under nitrogen atmosphere. To the above mixture was added Ammonium carbonate (309 mg, 3.22 mmol, 2.3 equiv) at 25° C. The resulting mixture was stirred at 25° C. for an additional 1 h. The desired product could be detected by LCMS. The resulting mixture was concentrated under reduced pressure. The resulting mixture was diluted with acetonitrile (100 mL). The resulting mixture was filtered; the filter cake was washed with acetonitrile (3×50 mL). The filtrate was concentrated under reduced pressure. The residue was purified by reverse phase flash with the following conditions (column, C18; mobile phase, MeCN in Water (10 mmol/L NH₄HCO₃), 20% to 80% gradient in 20 min, hold at 38%; detector, UV 254/220 nm) to afford the crude product. The crude product was purified by Prep-HPLC with the following conditions (Column: XBridge Prep OBD C18 Column 30*150 mm, 5 μm; Mobile Phase A: 10 mmol/L NH₄HCO₃+0.05% NH₃H₂O), Mobile Phase B: ACN; Flow rate: 60 mL/min; Gradient: 5% B to 5% B in 1 min, 5% B to 13% B in 2 min, 13% to 30% B in 11 min; Wave Length: 254 nm/220 nm; RT1 (min): 10.23) to afford 2-cyclopropyl-4-({4-[imino ((2H3)methyl) oxo-lambda6-sulfanyl]phenyl}methyl)-6-isopropyl-[1,2,4]triazolo[1,5-a]pyrimidin-7-one.

ESI-MS m/z=389.20 [M+H]⁺; Calculated: 388.2.

¹H NMR (400 MHZ, DMSO-d₆) δ 8.03 (s, 1H), 7.94-7.86 (m, 2H), 7.61-7.51 (m, 2H), 5.41 (s, 2H), 4.19 (s, 1H), 3.04-2.93 (m, 1H), 2.07-1.99 (m, 1H), 1.18 (d, J=6.9 Hz, 6H), 1.04-0.95 (m, 2H), 0.93-0.85 (m, 2H).

### Step 6: Rel-(R)-2-cyclopropyl-6-isopropyl-4-(4-(S-(methyl-d₃) sulfonimidoyl)benzyl)-[1,2,4]triazolo[1,5-a]pyrimidin-7(4H)-one

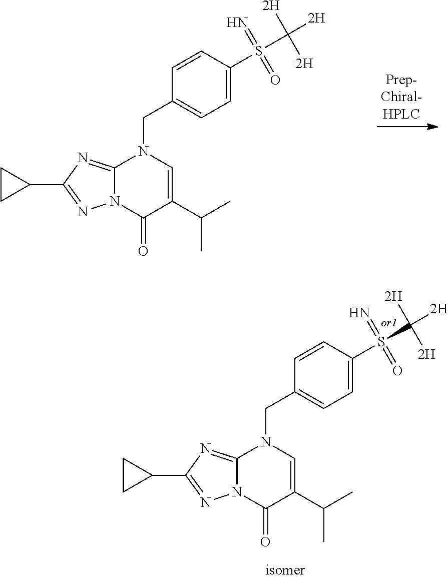

The 2-cyclopropyl-4-({4-[imino ((2H3)methyl) oxo-lambda6-sulfanyl]phenyl}methyl)-6-isopropyl-[1,2,4]triazolo[1,5-a]pyrimidin-7-one (100 mg, 0.26 mmol, 1.0 equiv) was separated by PREP-CHIRAL-HPLC with the following conditions (Column: CHIRALPAK-IK, 3*25 mm, 5 μm; Mobile Phase A: Hex (0.1% DEA)-HPLC, Mobile Phase B: EtOH; Flow rate: 40 mL/min; Gradient: isocratic 50; Wave Length: 218/284 nm; RT1 (min): 16.712; RT2 (min): 21.662; Sample Solvent: ETOH:DCM=1:1; Injection Volume: 0.6 mL; Number Of Runs: 5), the pure factions were concentrated under vacuum then lyophilized to afford rel-(R)-2-cyclopropyl-6-isopropyl-4-(4-(S-(methyl-da) sulfonimidoyl)benzyl)-[1,2,4]triazolo[1,5-a]pyrimidin-7(4H)-one.

ESI-MS m/z=389.15 [M+H]⁺; Calculated: 388.2.

¹H NMR (400 MHZ, DMSO-d₆) δ 8.04 (s, 1H), 7.95-7.85 (m, 2H), 7.61-7.50 (m, 2H), 5.41 (s, 2H), 4.19 (s, 1H), 3.06-2.91 (m, 1H), 2.09-1.98 (m, 1H), 1.19 (d, J=6.9 Hz, 6H), 1.04-0.96 (m, 2H), 0.93-0.86 (m, 2H).

Total proton count from structure: 20.

Total proton count from spectrum: 20.

### Compound 52: 4-((1-Imino-1-oxido-1/4-thiochroman-6-yl)methyl)-6-isopropyl-2-methyl-[1,2,4]triazolo[1,5-a]pyrimidin-7(4H)-one

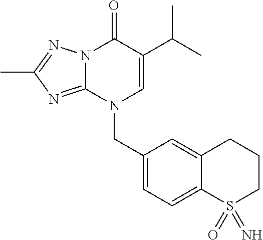 [SMILES: Cc1nc2n(Cc3ccc4c(c3)CCCS4(=N)=O)cc(C(C)C)c(=O)n2n1]

### Step 1: 6-Bromothiochromane

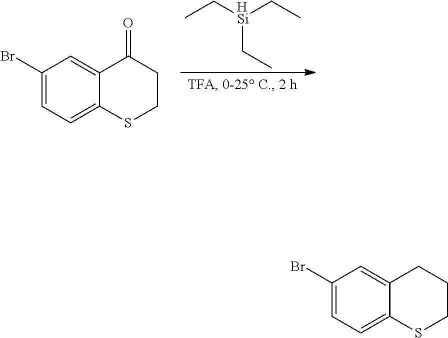

To a stirred solution of 6-bromothiochroman-4-one (10.00 g, 41.1 mmol, 1.0 equiv) in TFA (200 mL) was added triethylsilane (47.83 g, 411.3 mmol, 10.0 equiv) dropwise at 0° C. under nitrogen atmosphere. The resulting mixture was stirred for 2 h at 25° C. under nitrogen atmosphere. The desired product could be detected by TLC (100% PE, Rf=0.5). The resulting mixture was diluted with water (300 mL). The resulting mixture was extracted with CH₂Cl₂ (3×300 mL). The combined organic layers were washed with brine (500 mL), and dried over anhydrous Na₂SO₄. After filtration, the filtrate was concentrated under reduced pressure. The residue was purified by silica gel column chromatography, eluted with PE to afford 6-bromothiochromane.

¹H NMR (400 MHZ, DMSO-d₆) δ 7.31-7.26 (m, 1H), 7.22 (dd, J=8.4, 2.3 Hz, 1H), 7.00 (d, J=8.4 Hz, 1H), 3.05-2.98 (m, 2H), 2.80-2.72 (m, 2H), 2.02-1.92 (m, 2H).

### Step 2: Thiochroman-6-ylmethanol

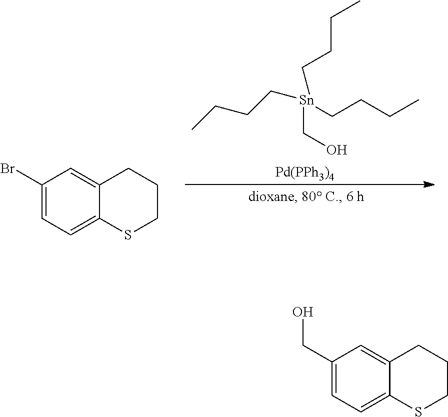

To a stirred solution of 6-bromothiochromane (6.00 g, 26.2 mmol, 1.0 equiv) and (tributylstannyl)methanol (16.82 g, 52.4 mmol, 2.0 equiv) in dioxane (60 mL) was added Pd(PPh₃)₄ (3.03 g, 2.62 mmol, 0.1 equiv) at 25° C. The resulting mixture was stirred for 6 h at 80° C. under nitrogen atmosphere. The desired product could be detected by LCMS. The mixture was allowed to cool down to 25° C. The reaction was quenched by the addition of sat. KF (aq.) (200 mL) at 25° C. The resulting mixture was filtered; the filter cake was washed with EtOAc (3×20 mL). The filtrate was extracted with EtOAc (3×200 mL). The combined organic layers were washed with brine (500 mL), and dried over anhydrous Na₂SO₄. After filtration, the filtrate was concentrated under reduced pressure. The residue was purified by reversed-phase flash chromatography with the following conditions: column, C18; mobile phase, MeCN in Water (10 mmol/L NH₄HCO₃), 30% to 50% gradient in 15 min; detector, UV 254 nm to afford thiochroman-6-ylmethanol.

ESI-MS m/z=162.90 [M−H₂O+H]⁺; Calculated: 180.1.

### Step 3: 6-(Hydroxymethyl)-1-imino-1/4-thiochromane 1-oxide

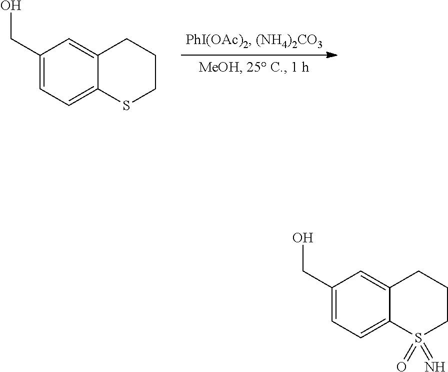 [SMILES: OCc1ccc2c(c1)CCCS2 ; N=S1(=O)CCCc2cc(CO)ccc21]

To a stirred solution of thiochroman-6-ylmethanol (1.20 g, 6.66 mmol, 1.0 equiv) and (diacetoxyiodo)benzene (9.86 g, 30.62 mmol, 4.6 equiv) in MeOH (12 mL) was added (NH₄)₂CO₃ (1.92 g, 19.97 mmol, 3.0 equiv) at 25° C. The resulting mixture was stirred for 1 h at 25° C. under nitrogen atmosphere. The desired product could be detected by LCMS. The resulting mixture was concentrated under reduced pressure. The residue was purified by silica gel column chromatography, eluted with CH₂Cl₂/MeOH (10:1) to afford 6-(hydroxymethyl)-1-imino-1/4-thiochromane 1-oxide.

ESI-MS m/z=211.90 [M+H]⁺; Calculated: 211.1.

¹H NMR (400 MHZ, DMSO-d₆+D₂O) δ 7.80 (d, J=8.2 Hz, 1H), 7.38-7.32 (m, 1H), 7.23 (s, 1H), 5.39-5.22 (m, 1H), 4.55-4.48 (m, 2H), 3.42-3.27 (m, 2H), 3.01-2.91 (m, 2H), 2.38-2.21 (m, 2H).

### Step 4: 6-(Chloromethyl)-1-imino-1/4-thiochromane 1-oxide

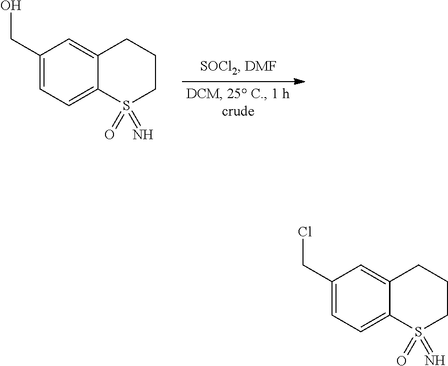 [SMILES: N=S1(=O)CCCc2cc(CO)ccc21 ; N=S1(=O)CCCc2cc(CCl)ccc21]

To a stirred solution of 6-(hydroxymethyl)-1-imino-1/4-thiochromane 1-oxide (400 mg, 1.89 mmol, 1.0 equiv) and DMF (14 mg, 0.19 mmol, 0.10 equiv) in DCM (8 mL) was added SOCl₂ (676 mg, 5.68 mmol, 3.0 equiv) dropwise at 0° C. The resulting mixture was stirred for 1 h at 25° C. under nitrogen atmosphere. The desired product could be detected by LCMS. The resulting mixture was concentrated under reduced pressure to afford 6-(chloromethyl)-1-imino-1/4-thiochromane 1-oxide (crude).

ESI-MS m/z=230.00 [M+H]⁺; Calculated: 229.0.

### Step 5: 4-((1-Imino-1-oxido-1/4-thiochroman-6-yl)methyl)-6-isopropyl-2-methyl-[1,2,4]triazolo[1,5-a]pyrimidin-7(4H)-one

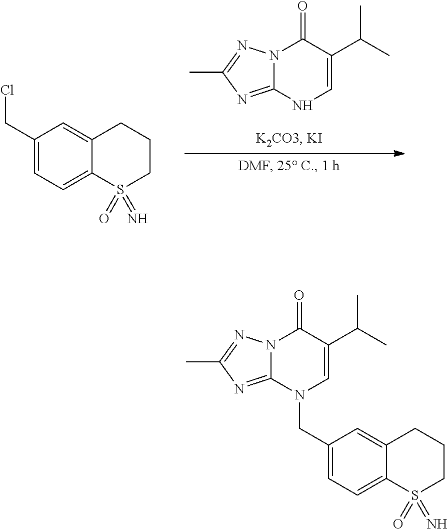 [SMILES: N=S1(=O)CCCc2cc(CCl)ccc21 ; Cc1nc2[nH]cc(C(C)C)c(=O)n2n1 ; Cc1nc2n(Cc3ccc4c(c3)CCCS4(=N)=O)cc(C(C)C)c(=O)n2n1]

To a stirred solution of 6-(chloromethyl)-1-imino-1l4-thiochromane 1-oxide (400 mg, 1.74 mmol, 1.0 equiv) and 6-isopropyl-2-methyl-4H-[1,2,4]triazolo[1,5-a]pyrimidin-7-one (402 mg, 2.09 mmol, 1.2 equiv) in DMF (8 mL) were added K₂CO₃ (481 mg, 3.48 mmol, 2.0 equiv) and KI (289 mg, 1.74 mmol, 1.0 equiv) at 25° C. The resulting mixture was stirred for 1 h at 25° C. under nitrogen atmosphere. The desired product could be detected by LCMS. The resulting mixture was diluted with water (100 mL). The resulting mixture was extracted with CH₂Cl₂/IPA (3×100 mL). The combined organic layers were washed with brine (200 mL), and dried over anhydrous Na₂SO₄. After filtration, the filtrate was concentrated under reduced pressure. The crude product (400 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min mL/min; Gradient: 20% B to 45% B in 7 min; Wave Length: 254 nm/220 nm nm; RT1 (min): 6.6), the pure fraction was concentrated then lyophilized to afford 4-((1-imino-1-oxido-1/4-thiochroman-6-yl)methyl)-6-isopropyl-2-methyl-[1,2,4]triazolo[1,5-a]pyrimidin-7(4H)-one.

ESI-MS m/z=386.10 [M+H]⁺; Calculated: 385.2.

¹H NMR (400 MHZ, DMSO-d₆) δ 8.02 (s, 1H), 7.80 (d, J=8.2 Hz, 1H), 7.33 (d, 1H), 7.25 (br, 1H), 5.36 (s, 2H), 4.43 (s, 1H), 3.39-3.25 (m, 2H), 3.06-2.96 (m, 1H), 2.96-2.86 (m, 2H), 2.34 (s, 3H), 2.29-2.17 (m, 2H), 1.26-1.14 (m, 6H).

Total proton count from structure: 23.

Total proton count from spectrum: 23.

### Compound 53: 2-Cyclopropyl-4-((4-imino-4-oxido-3,4-dihydro-2H-4lambda4-benzo[b][1,4]oxathiin-7-yl)methyl)-6-isopropyl-[1,2,4]triazolo[1,5-a]pyrimidin-7(4H)-one

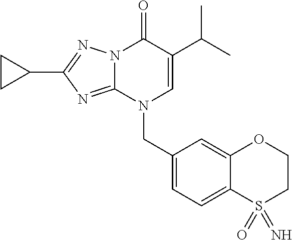 [SMILES: CC(C)c1cn(Cc2ccc3c(c2)OCCS3(=N)=O)c2nc(C3CC3)nn2c1=O]

### Step 1: Methyl 3-(2-bromoethoxy)-4-iodobenzoate

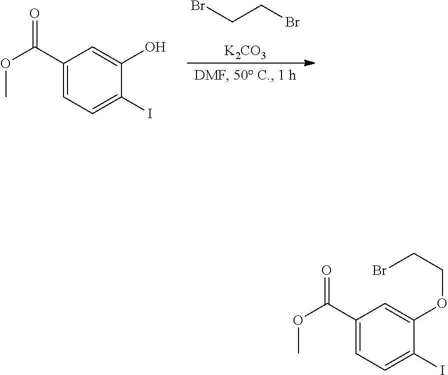

To a stirred mixture of methyl 3-hydroxy-4-iodobenzoate (30.00 g, 107.8 mmol, 1.0 equiv) and K₂CO₃ (44.74 g, 323.6 mmol, 3.0 equiv) in DMF (300 mL) was added 1,2-dibromoethane (40.54 g, 215.7 mmol, 2.0 equiv) at 25° C. under nitrogen atmosphere. The resulting mixture was stirred for 1 h at 50° C. under nitrogen atmosphere. The mixture was allowed to cool down to 25° C. The desired products could be detected by TLC (PE/EA=6:1, Rf=0.5). The resulting mixture was diluted with EtOAc (600 mL). The organic layer was washed with water (3×200 mL), and dried over anhydrous Na₂SO₄. After filtration, the filtrate was concentrated under reduced pressure. The residue was purified by silica gel column chromatography, eluted with PE/EA (12:1) to afford methyl 3-(2-bromoethoxy)-4-iodobenzoate. 1H NMR (400 MHZ, DMSO-d₆) δ 7.97 (d, J=8.1 Hz, 1H). 7.42 (d, J=1.9 Hz, 1H), 7.34 (dd, J=8.0, 1.8 Hz, 1H), 4.50-4.43 (m, 2H), 3.88-3.80 (m, 5H).

### Step 2: Methyl 3-(2-(acetylthio) ethoxy)-4-iodobenzoate

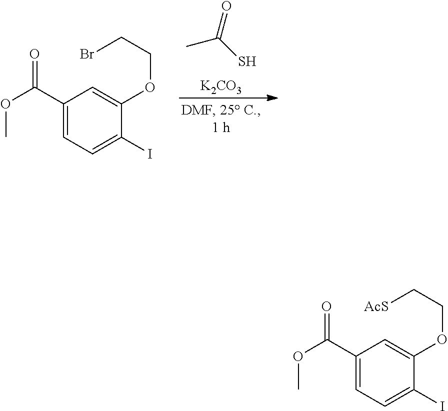

To a stirred mixture of methyl 3-(2-bromoethoxy)-4-iodobenzoate (14.00 g, 36.3 mmol, 1.0 equiv) and K₂CO₃ (10.05 g, 72.7 mmol, 2.0 equiv) in DMF (100 mL) was added ethanethioic S-acid (3.32 g, 43.6 mmol, 1.2 equiv) at 25° C. under nitrogen atmosphere. The resulting mixture was stirred for 1 h at 25° C. under nitrogen atmosphere. The desired product could be detected by LCMS. The resulting mixture was diluted with EtOAc (500 mL). The residue was washed with water (3×60 mL), and dried over anhydrous Na₂SO₄. After filtration, the filtrate was concentrated under reduced pressure. The residue was purified by silica gel column chromatography, eluted with PE/EA (0-30% in 25 min) to afford methyl 3-(2-(acetylthio) ethoxy)-4-iodobenzoate.

ESI-MS m/z=380.80 [M+H]⁺; Calculated: 380.0.

¹H NMR (400 MHz, DMSO-d₆) δ 7.95 (d, J=8.0 Hz, 1H), 7.42 (d, J=1.8 Hz, 1H), 7.33 (dd, J=8.1, 1.8 Hz, 1H), 4.23 (t, J=6.2 Hz, 2H), 3.85 (s, 3H), 3.29 (t, J=6.2 Hz, 2H), 2.38 (s, 3H).

### Step 3: Methyl 2,3-dihydrobenzo[b][1,4]oxathiine-7-carboxylate

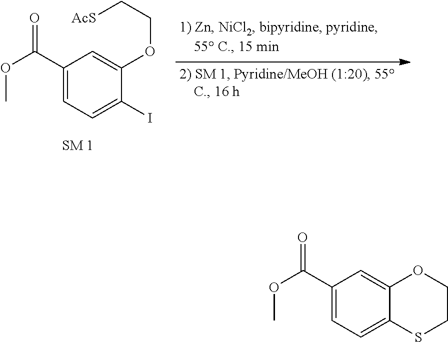

To a stirred mixture of zinc (1.72 g, 26.3 mmol, 2.0 equiv) and nickel chloride (852 mg, 6.57 mmol, 0.5 equiv) in pyridine (5 mL) was added bipyridine (1.23 g, 7.89 mmol, 0.6 equiv) at 25° C. under nitrogen atmosphere. The resulting mixture was stirred for 15 min at 55° C. under nitrogen atmosphere. The mixture was allowed to cool down to 25° C. To the above mixture was added methyl 3-(2-(acetylthio) ethoxy)-4-iodobenzoate (5.00 g, 13.1 mmol, 1.0 equiv) in MeOH/Pyridine (20:1, 105 mL) at 25° C. The resulting mixture was stirred for an additional 16 h at 55° C. The desired products could be detected by LCMS. The resulting mixture was poured into water (100 mL). The aqueous layer was extracted with EA (2×400 mL). The combined organic layers were washed with 1N HCl (50 mL) and brine (100 mL), and dried over anhydrous Na₂SO₄. After filtration, the filtrate was concentrated under reduced pressure. The residue was purified by silica gel column chromatography, eluted with PE/EA (0-25% in 20 min) to afford methyl 2,3-dihydrobenzo[b][1,4]oxathiine-7-carboxylate.

ESI-MS m/z=210.90 [M+H]⁺; Calculated: 210.0.

¹H NMR (400 MHZ, DMSO-d₆) δ 7.42 (dd, J=8.2, 1.8 Hz, 1H), 7.31 (d, J=1.8 Hz, 1H), 7.22 (d, J=8.2 Hz, 1H), 4.43-4.34 (m, 2H), 3.81 (s, 3H), 3.28-3.21 (m, 2H).

### Step 4: (2,3-Dihydrobenzo[b][1,4]oxathiin-7-yl)methanol

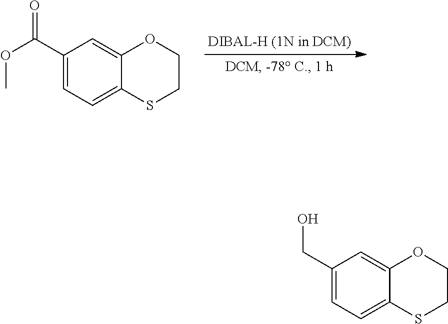

To a stirred mixture of methyl 2,3-dihydrobenzo[b][1,4]oxathiine-7-carboxylate (1.20 g, 5.70 mmol, 1.0 equiv) in DCM (12 mL) was added 1N DIBAL-H in DCM (1.62 g, 11.4 mmol, 2.0 equiv) dropwise at −78° C. under nitrogen atmosphere. The resulting mixture was stirred for 1 h at −78° C. under nitrogen atmosphere. The desired product could be detected by LCMS. The mixture was allowed to warm up to 0° C. The reaction was quenched by the addition of MeOH (5 mL) at 0° C. To the resulting mixture was added ice water (80 mL) and the mixture was stirred for 30 min. The mixture was extracted with EtOAc (3×80 mL). The combined organic layers were dried over anhydrous Na₂SO₄. After filtration, the filtrate was concentrated under reduced pressure. The residue was purified by silica gel column chromatography, eluted with PE/EA (1:1) to afford 2,3-dihydro-1,4-benzoxathiin-7-ylmethanol.

ESI-MS m/z=164.85 [M−H₂O+H]⁺; Calculated: 182.0.

¹H NMR (400 MHZ, DMSO-d₆)· 6.98 (d, J=7.9 Hz, 1H), 6.83-6.73 (m, 2H), 5.11 (t, J=5.8 Hz, 1H), 4.40-4.30 (m, 4H), 3.19-3.12 (m, 2H).

### Step 5:7-(Hydroxymethyl)-4-imino-3,4-dihydro-2H-4 lambda4-benzo[b][1,4]oxathiine 4-oxide

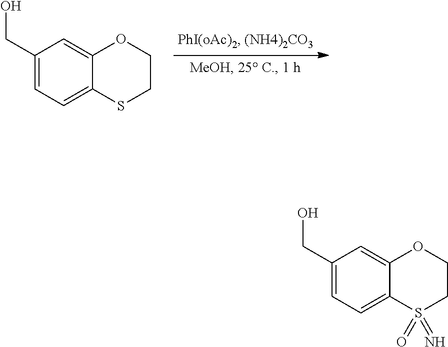 [SMILES: OCc1ccc2c(c1)OCCS2 ; N=S1(=O)CCOc2cc(CO)ccc21]

To a stirred mixture of 2,3-dihydro-1,4-benzoxathiin-7-ylmethanol (800 mg, 4.39 mmol, 1.0 equiv) and PhI(OAc)₂ (3.25 g, 10.0 mmol, 2.3 equiv) in MeOH (8 mL) was added ammonium carbonate (632 mg, 6.58 mmol, 1.5 equiv) at 25° C. under nitrogen atmosphere. The resulting mixture was stirred for 1 h at 25° C. under nitrogen atmosphere. The desired product could be detected by LCMS. The resulting mixture was concentrated under reduced pressure. The residue was purified by silica gel column chromatography, eluted with CH₂Cl₂/MeOH (0-15% in 25 min) to afford 7-(hydroxymethyl)-4-imino-3,4-dihydro-2H-4 lambda4-benzo[b][1,4]oxathiine 4-oxide.

ESI-MS m/z=213.90 [M+H]⁺; Calculated: 213.0.

¹H NMR (400 MHZ, DMSO-d₆) δ 7.71 (d, J=8.1 Hz, 1H), 7.04 (dd, J=8.2, 1.5 Hz, 1H), 6.91 (d, J=1.4 Hz, 1H), 5.34 (t, J=5.8 Hz, 1H), 4.77-4.62 (m, 3H), 4.47 (d, J=5.8 Hz, 2H), 3.63-3.48 (m, 2H).

### Step 6:7-(Bromomethyl)-4-imino-3,4-dihydro-2H-4 lambda4-benzo[b][1,4]oxathiine 4-oxide

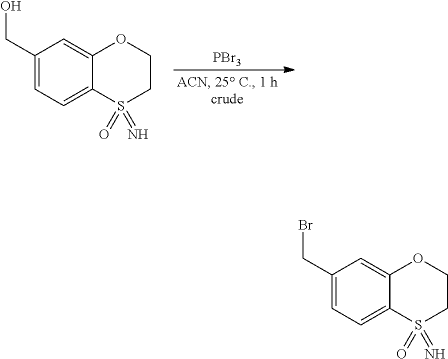

To a stirred solution of 7-(hydroxymethyl)-4-imino-3,4-dihydro-2H-4 lambda4-benzo[b][1,4]oxathiine 4-oxide (500 mg, 2.34 mmol, 1.0 equiv) in ACN (5 mL) was added PBr₃ (634 mg, 2.34 mmol, 1.0 equiv) dropwise at 25° C. under nitrogen atmosphere. The resulting mixture was stirred for 1 h at 25° C. under concentrated under reduced pressure to afford 7-(bromomethyl)-4-imino-3,4-dihydro-2H-4 lambda4-benzo[b][1,4]oxathiine 4-oxide (crude).

ESI-MS m/z=275.90, 277.90 [M+H]⁺; Calculated: 275.0, 277.0.

### Step 7: 2-Cyclopropyl-4-((4-imino-4-oxido-3,4-dihydro-2H-4 lambda4-benzo[b][1,4]oxathiin-7-yl)methyl)-6-isopropyl-[1,2,4]triazolo[1,5-a]pyrimidin-7(4H)-one

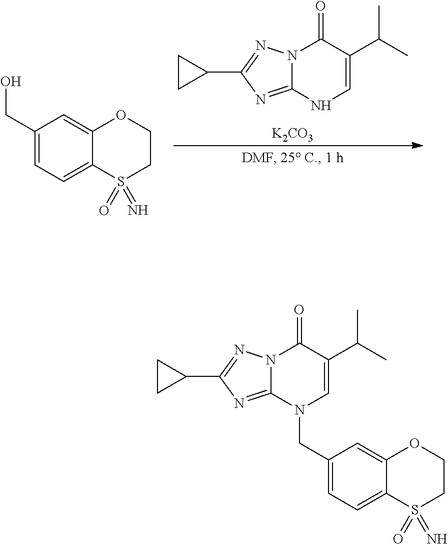 [SMILES: N=S1(=O)CCOc2cc(CO)ccc21 ; CC(C)c1c[nH]c2nc(C3CC3)nn2c1=O ; "K2CO3" ; CC(C)c1cn(Cc2ccc3c(c2)OCCS3(=N)=O)c2nc(C3CC3)nn2c1=O]

To a stirred mixture of 2-cyclopropyl-6-isopropyl-4H-[1,2,4]triazolo[1,5-a]pyrimidin-7-one (300 mg, 1.37 mmol, 1.0 equiv) and 7-(bromomethyl)-4-imino-3,4-dihydro-2H-4 lambda4-benzo[b][1,4]oxathiine 4-oxide (455 mg, 1.65 mmol, 1.2 equiv) in DMF (3 mL) was added K₂CO₃ (569 mg, 4.12 mmol, 3.0 equiv) at 25° C. under nitrogen atmosphere. The resulting mixture was stirred for 1 h at 25° C. under nitrogen atmosphere. The desired product could be detected by LCMS. The resulting mixture was filtered, and the filter cake was washed with EtOAc (3×3 mL). The filtrate was concentrated under reduced pressure. The residue was purified by reversed-phase flash chromatography with the following conditions: column, C18; mobile phase, MeCN in Water (10 mmol/L NH₄HCO₃), 15% to 45% gradient in 20 min; detector, UV 220 nm to afford the crude product. The crude product was purified by Prep-HPLC with the following conditions (Column: XSelect CSH Prep C18 OBD Column, 30*150 mm, 5 m; Mobile Phase A: Water (0.1% FA), Mobile Phase B: ACN; Flow rate: 60 mL/min mL/min; Gradient: 14% B to 34% B in 9 min; Wave Length: 254 nm/220 nm nm; RT1 (min): 9.22), the pure fraction was concentrated under reduced pressure and then lyophilized to afford 2-cyclopropyl-4-((4-imino-4-oxido-3,4-dihydro-2H-4 lambda4-benzo[b][1,4]oxathiin-7-yl)methyl)-6-isopropyl-[1,2,4]triazolo[1,5-a]pyrimidin-7(4H)-one.

ESI-MS m/z=414.15 [M+H]⁺; Calculated: 413.2.

¹H NMR (400 MHZ, DMSO-d₆) δ 8.01 (s, 1H), 7.77 (d, J=8.2 Hz, 1H), 7.12 (dd, J=8.3, 1.7 Hz, 1H), 7.02 (d, J=1.6 Hz, 1H), 5.31 (s, 2H), 4.81 (s, 1H), 4.78-4.63 (m, 2H), 3.67-3.51 (m, 2H), 3.06-2.91 (m, 1H), 2.11-2.00 (m, 1H), 1.19 (d, J=7.0 Hz, 3H), 1.19 (d, J=7.0 Hz, 3H), 1.06-0.97 (m, 2H), 1.00-0.86 (m, 2H).

Total proton count from structure: 23.

Total proton count from spectrum: 23.

### Compound 54—4-(4-(Cyclopropanesulfonimidoyl)benzyl)-2-cyclopropyl-6-isopropyl-[1,2,4]triazolo[1,5-a]pyrimidin-7(4H)-one

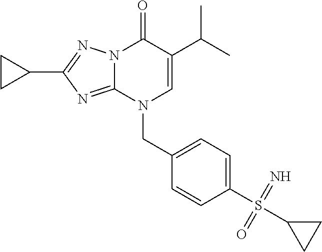 [SMILES: CC(C)c1cn(Cc2ccc(S(=N)(=O)C3CC3)cc2)c2nc(C3CC3)nn2c1=O]

### Step 1: [4-(Cyclopropylsulfanyl)phenyl]methanol

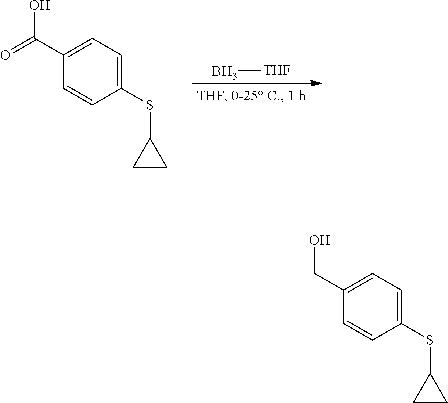

To a solution of 4-(cyclopropylsulfanyl)benzoic acid (1.90 g, 9.78 mmol, 1.0 equiv) in THF (28 mL) under nitrogen atmosphere was added 1M BH₃-THF (29 mL, 29.3 mmol, 3.0 equiv) dropwise at 0° C. The resulting mixture was stirred for 1 h at 25° C. The desired product could be detected by LCMS. The reaction was quenched with water (150 mL) at 0° C. The resulting mixture was extracted with CH₂Cl₂ (3×150 mL). The combined organic layers were washed with brine (1×300 mL), and dried over anhydrous Na₂SO₄. After filtration, the filtrate was concentrated under reduced pressure. The residue was purified by silica gel column chromatography, and eluted with PE/EA (0% to 30% gradient in 20 min) to afford [4-(cyclopropylsulfanyl)phenyl]methanol. ESI-MS m/z=163.05 [M-17]⁺; Calculated: 180.1.

¹H NMR (400 MHZ, DMSO-d₆) δ 7.35-7.23 (m, 4H), 5.16 (s, 1H), 4.46 (s, 2H), 2.28-2.20 (m, 1H), 1.1-1.02 (m, 2H), 0.74-0.52 (m, 2H).

### Step 2: Cyclopropyl[4-(hydroxymethyl)phenyl]imino-lambda6-sulfanylone

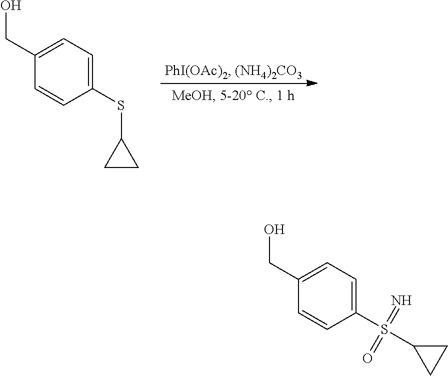 [SMILES: OCc1ccc(SC2CC2)cc1 ; N=S(=O)(c1ccc(CO)cc1)C1CC1]

To a mixture of [4-(cyclopropylsulfanyl)phenyl]methanol (700 mg, 3.88 mmol, 1.0 equiv) and (diacetoxyiodo)benzene (4.38 g, 13.5 mmol, 3.5 equiv) in MeOH (14.00 mL) was added [4-(cyclopropylsulfanyl)phenyl]methanol (700 mg, 3.88 mmol, 1.0 equiv) in portions at 10° C. The resulting mixture was stirred for an additional 1 h between 5 and 20° C. The desired products could be detected by LCMS. The resulting mixture was concentrated under reduced pressure. The residue was purified by silica gel column chromatography, and eluted with CH₂Cl₂/MeOH (0% to 10% gradient in 20 min) to afford cyclopropyl[4-(hydroxymethyl)phenyl]imino-lambda6-sulfanylone.

ESI-MS m/z=212.15 [M+H]⁺; Calculated: 211.1.

¹H NMR (400 MHZ, DMSO-d₆) δ 7.88-7.79 (m, 2H), 7.51 (d, J=8.2 Hz, 2H), 4.59 (s, 2H), 4.21 (d, J=6.5 Hz, 1H), 4.25-4.03 (br, 1H), 2.71-2.62 (m, 1H), 1.15-1.04 (m, 1H), 0.98-0.80 (m, 3H).

### Step 3: [4-(Chloromethyl)phenyl](cyclopropyl)imino-lambda6-sulfanylone

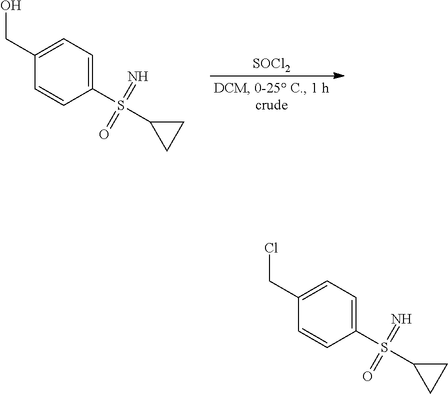 [SMILES: N=S(=O)(c1ccc(CO)cc1)C1CC1 ; "SOCl2" ; N=S(=O)(c1ccc(CCl)cc1)C1CC1]

To a stirred solution of [4-(hydroxymethyl)phenyl](imino)isopropyl-lambda6-sulfanone (400 mg, 1.87 mmol, 1.0 equiv) in DCM (8.00 mL) was added SOCl₂ (446 mg, 3.75 mmol, 2.0 equiv) dropwise at 0° C. The resulting mixture was stirred for an additional 1 h at 25° C. The resulting mixture was concentrated under reduced pressure. This resulted in [4-(chloromethyl)phenyl](cyclopropyl)imino-lambda6-sulfanylone (crude).

ESI-MS m/z=230.20 [M+H]⁺; Calculated: 229.0.

### Step 4: 4-(4-(Cyclopropanesulfonimidoyl)benzyl)-2-cyclopropyl-6-isopropyl-[1,2,4]triazolo[1,5-a]pyrimidin-7(4H)-one

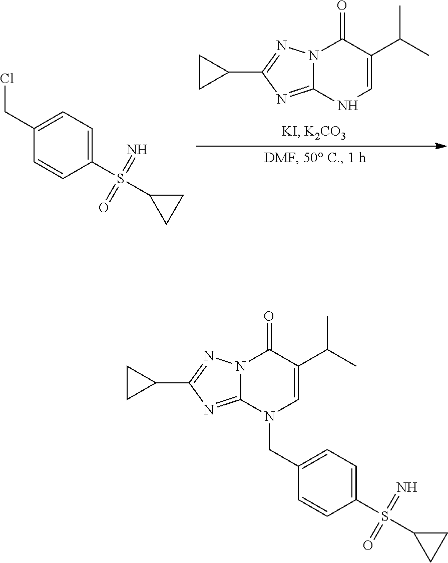 [SMILES: N=S(=O)(c1ccc(CCl)cc1)C1CC1 ; CC(C)c1c[nH]c2nc(C3CC3)nn2c1=O ; CC(C)c1cn(Cc2ccc(S(=N)(=O)C3CC3)cc2)c2nc(C3CC3)nn2c1=O]

A mixture of [4-(chloromethyl)phenyl](cyclopropyl)imino-lambda6-sulfanylone (400 mg, 1.70 mmol, 1.0 equiv), 2-cyclopropyl-6-isopropyl-4H-[1,2,4]triazolo[1,5-a]pyrimidin-7-one (380 mg, 1.74 mmol, 1.0 equiv), KI (289 mg, 1.74 mmol, 1.0 equiv) and K₂CO₃ (1.20 g, 8.70 mmol, 5.0 equiv) in DMF (8.00 mL) was stirred for 1 h at 50° C. The desired product could be detected by LCMS. The reaction was quenched with water (50 mL) at 25° C. The resulting mixture was extracted with CH₂Cl₂/IPA (5/1) (3×40 mL). The combined organic layers were washed with brine (3×50 mL), and dried over anhydrous Na₂SO₄. After filtration, the filtrate was concentrated under reduced pressure. The crude product (500 mg) was purified by Prep-HPLC with the following conditions (Column: YMC-Actus Triart C18 ExRS 30*150 mm, 5 m; Mobile Phase A: Water (10 mmol/L NH₄HCO₃), Mobile Phase B: ACN; Flow rate: 60 mL/min; Gradient: 20% B to 36% B in 10 min; Wave Length: 254 nm/220 nm; RT1 (min): 9.85), and the pure factions were concentrated under vacuum then lyophilized to afford 4-(4-(cyclopropanesulfonimidoyl)benzyl)-2-cyclopropyl-6-isopropyl-[1,2,4]triazolo[1,5-a]pyrimidin-7(4H)-one.

ESI-MS m/z=412.10 [M+H]⁺; Calculated: 411.2.

¹H NMR (400 MHZ, DMSO-d₆) δ 8.05 (s, 1H), 7.92-7.83 (m, 2H), 7.60-7.52 (m, 2H), 5.49-5.36 (m, 2H), 4.19 (s, 1H), 3.06-2.93 (m, 1H), 2.70-2.58 (m, 1H), 2.10-1.97 (m, 1H), 1.23-1.09 (m, 6H), 1.15-0.80 (m, 8H).

Total proton count from structure: 25.

Total proton count from spectrum: 25.

### Compound 55-Rel-(S)-4-(4-(cyclopropanesulfonimidoyl)benzyl)-2-cyclopropyl-6-isopropyl-[1,2,4]triazolo[1,5-a]pyrimidin-7(4H)-one

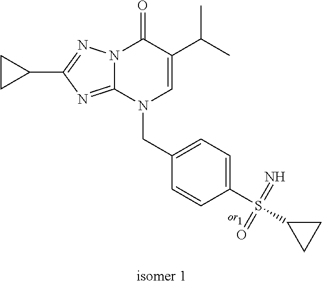 [SMILES: CC(C)c1cn(Cc2ccc([S@@](=N)(=O)C3CC3)cc2)c2nc(C3CC3)nn2c1=O]

### Step 1: Rel-(S)-4-(4-(cyclopropanesulfonimidoyl)benzyl)-2-cyclopropyl-6-isopropyl-[1,2,4]triazolo[1,5-a]pyrimidin-7(4H)-one

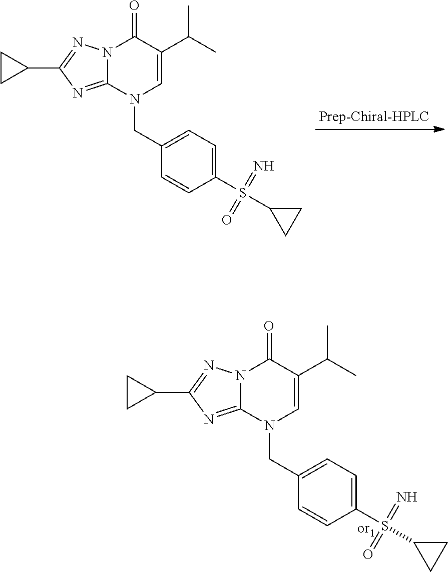 [SMILES: CC(C)c1cn(Cc2ccc(S(=N)(=O)C3CC3)cc2)c2nc(C3CC3)nn2c1=O ; CC(C)c1cn(Cc2ccc([S@@](=N)(=O)C3CC3)cc2)c2nc(C3CC3)nn2c1=O]

The racemate (150 mg) was separated by PREP-CHIRAL-HPLC with the following conditions (Column: CHIRAL ART Cellulose-SZ, 3*25 cm, 5 μm; Mobile Phase A: Hex (10 mM NH₃-MeOH), Mobile Phase B: ETOH; Flow rate: 40 mL/min; Gradient: isocratic 50; Wave Length: 202/220 nm; RT1 (min): 14; Sample Solvent: EtOH: MeOH=2:1-HPLC; Number Of Runs: 5), and the pure factions were concentrated under vacuum then lyophilized to afford rel-(S)-4-(4-(cyclopropanesulfonimidoyl)benzyl)-2-cyclopropyl-6-isopropyl-[1,2,4]triazolo[1,5-a]pyrimidin-7(4H)-one.

ESI-MS m/z=412.20 [M+H]⁺; Calculated: 411.2.

¹H NMR (400 MHZ, DMSO-d₆) δ 8.03 (s, 1H), 7.87 (d, J=8.2 Hz, 2H), 7.55 (d, J=8.1 Hz, 2H), 5.41 (s, 2H), 4.20 (s, 1H), 3.04-2.92 (m, 1H), 2.68-2.57 (m, 1H), 2.11-1.97 (m, 1H), 1.19 (d, J=6.9 Hz, 6H), 1.13-1.04 (m, 1H), 1.03-0.97 (m, 2H), 0.97-0.86 (m, 5H).

Total proton count from structure: 25.

Total proton count from spectrum: 25.

### Compound 56 Rac: 2-Cyclopropyl-4-(4-(ethylsulfonimidoyl)benzyl)-6-isopropyl-[1,2,4]triazolo[1,5-a]pyrimidin-7(4H)-one

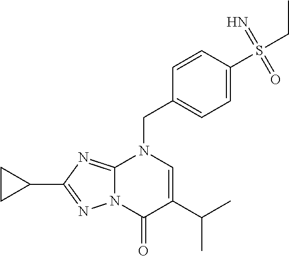 [SMILES: CCS(=N)(=O)c1ccc(Cn2cc(C(C)C)c(=O)n3nc(C4CC4)nc23)cc1]

### Step 1: 4-(Ethylthio)benzoic acid

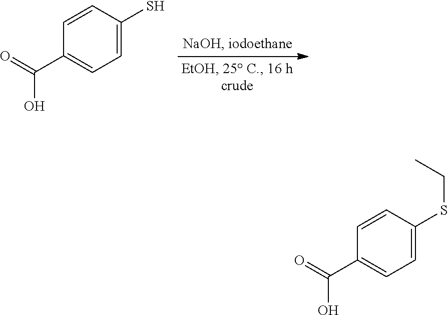

To a mixture of 2-mercaptobenzoic acid (3.00 g, 19.4 mmol, 1.0 equiv) and iodoethane (6.07 g, 38.9 mmol, 2.0 equiv) in EtOH (30 mL) was added NaOH (2.33 mL, 58.37 mmol, 3.0 equiv) in portions at 25° C. under nitrogen atmosphere. The resulting mixture was stirred for an additional 16 h at 25° C. The desired products could be detected by LCMS. The mixture was acidified to pH 4 with conc. HCl. The precipitated solids were collected by filtration and washed with water (2×50 mL). This resulted in 4-(ethylthio)benzoic acid (crude):

ESI-MS m/z=180.90 [M−H]⁻; Calculated: 182.0.

¹H NMR (400 MHZ, DMSO-d₆) δ 12.86 (s, 1H), 8.03-7.71 (m, 2H), 7.59-7.16 (m, 2H), 3.07 (q, J=7.3 Hz, 2H), 1.28 (t, J=7.3 Hz, 3H).

### Step 2: (4-(Ethylthio)phenyl)methanol

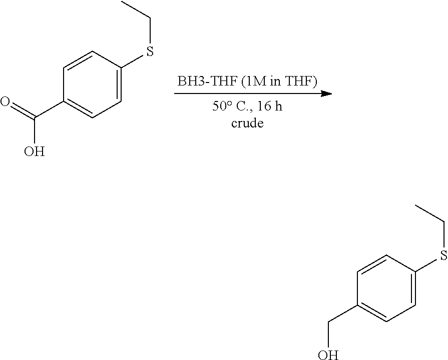

To a mixture of 4-(ethylthio)benzoic acid (3.00 g, 16.4 mmol, 1.0 equiv) in THF (30.00 mL) was added BH₃·THF (82.3 mL, 82.3 mmol, 5.0 equiv, 1 M in THF) in portions at 0° C. under nitrogen atmosphere. The resulting mixture was stirred for an additional 16 h at 50° C. The reaction was monitored by LCMS. The mixture was allowed to cool down to 25° C. The reaction was quenched with water (100 mL) at 0° C. The resulting mixture was extracted with EtOAc (3×100 mL). The combined organic layers were dried over anhydrous Na₂SO₄. After filtration, the filtrate was concentrated under reduced pressure to afford (4-(ethylthio)phenyl)methanol (crude).

ESI-MS m/z=151.03 [M-17]⁺; Calculated: 168.0.

### Step 3: Ethyl(4-(hydroxymethyl)phenyl)(imino)-l6-sulfanone

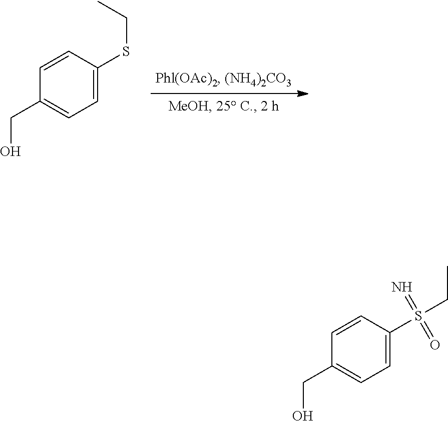

To a mixture of [4-(ethylsulfanyl)phenyl]methanol (3.00 g, 17.8 mmol, 1.0 equiv) and PhI(OAc)₂ (13.21 g, 41.0 mmol, 2.3 equiv) in MeOH (60.00 mL) was added (NH₄)₂CO₃ (2.57 g, 26.7 mmol, 1.5 equiv) in portions at 25° C. under nitrogen atmosphere. The resulting mixture was stirred for an additional 16 h at 25° C. The desired product could be detected by LCMS. The resulting mixture was concentrated under reduced pressure. The residue was purified by silica gel column chromatography, eluted with CH₂Cl₂/MeOH (10:1) to afford ethyl(4-(hydroxymethyl)phenyl)(imino)-l6-sulfanone.

ESI-MS m/z=200.00 [M+H]⁺; Calculated: 199.0.

¹H NMR (400 MHZ, DMSO-d6) δ 7.84 (t, J=7.4 Hz, 2H), 7.54 (t, J=7.0 Hz, 2H), 5.48 (s, 1H), 4.60 (s, 2H), 4.13 (s, 1H), 3.12 (dq, J=14.7, 7.5 Hz, 2H), 1.06 (q, J=7.3, 6.8 Hz, 3H).

### Step 4: [4-(bromomethyl)phenyl](ethyl)imino-lambda6-sulfanone

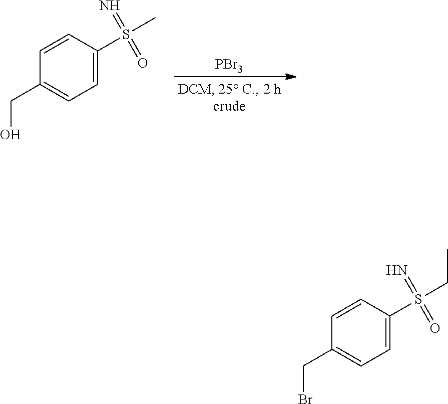

To a mixture of ethyl(4-(hydroxymethyl)phenyl)(imino)-l6-sulfanone (2.00 g, 10.0 mmol, 1.0 equiv) in DCM (20 mL) was added PBr₃ (5.43 g, 20.0 mmol, 2.0 equiv) in portions at 0° C. under nitrogen atmosphere. The resulting mixture was stirred for an additional 2 h at 25° C. The desired product could be detected by LCMS. The resulting mixture was concentrated under reduced pressure. The residue was purified by trituration with diethyl ether (3×100 mL) to afford [4-(bromomethyl)phenyl](ethyl)imino-lambda6-sulfanone (crude).

ESI-MS m/z=263.90, 262.10 [M+H]⁺; Calculated: 261.0, 263.0.

### Step 5: 2-Cyclopropyl-4-(4-(ethylsulfonimidoyl)benzyl)-6-isopropyl-[1,2,4]triazolo[1,5-a]pyrimidin-7(4H)-one

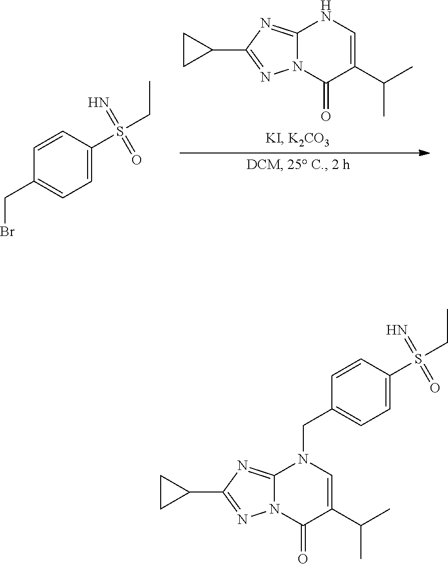

To a mixture of [4-(bromomethyl)phenyl](ethyl)imino-lambda6-sulfanone (500 mg, 1.90 mmol, 1.0 equiv) and 2-cyclopropyl-6-isopropyl-4H-[1,2,4]triazolo[1,5-a]pyrimidin-7-one (457 mg, 2.09 mmol, 1.1 equiv) in DMF (50.00 mL) were added K₂CO₃ (527 mg, 3.81 mmol, 2.0 equiv) in portions at 25° C. under nitrogen atmosphere. The resulting mixture was stirred for 2 h at 25° C. under nitrogen atmosphere. The desired product could be detected by LCMS. The resulting mixture was diluted with EtOAc (200 mL). The resulting mixture was washed with 3×50 ml of water. The crude product (300 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min mL/min; Gradient: 12% B to 38% B in 7 min; Wave Length: 254 nm/220 nm nm; RT1 (min): 6.72), the pure fraction was concentrated under reduced pressure and then lyophilized to afford 2-cyclopropyl-4-(4-(ethylsulfonimidoyl)benzyl)-6-isopropyl-[1,2,4]triazolo[1,5-a]pyrimidin-7(4H)-one.

ESI-MS m/z=400.05 [M+H]⁺; Calculated: 399.1.

¹H NMR (400 MHZ, DMSO-d₆) δ 8.03 (s, 1H), 7.85 (d, J=8.3 Hz, 2H), 7.56 (d, J=8.3 Hz, 2H), 5.42 (s, 2H), 4.17 (s, 1H), 3.15-3.06 (m, 2H), 3.06-2.91 (m, 1H), 2.09-1.98 (m, 1H), 1.18 (d, J=6.9 Hz, 3H), 1.18 (d, J=6.9 Hz, 3H), 1.07-0.93 (m, 5H), 0.92-0.84 (m, 2H).

### Compound 56A: Rel-(S)-2-cyclopropyl-4-(4-(ethylsulfonimidoyl)benzyl)-6-isopropyl-[1,2,4]triazolo[1,5-a]pyrimidin-7(4H)-one

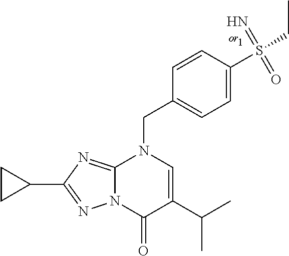 [SMILES: CC[S@](=N)(=O)c1ccc(Cn2cc(C(C)C)c(=O)n3nc(C4CC4)nc23)cc1]

### Step 1: Rel-(S)-2-cyclopropyl-4-(4-(ethylsulfonimidoyl)benzyl)-6-isopropyl-[1,2,4]triazolo[1,5-a]pyrimidin-7(4H)-one

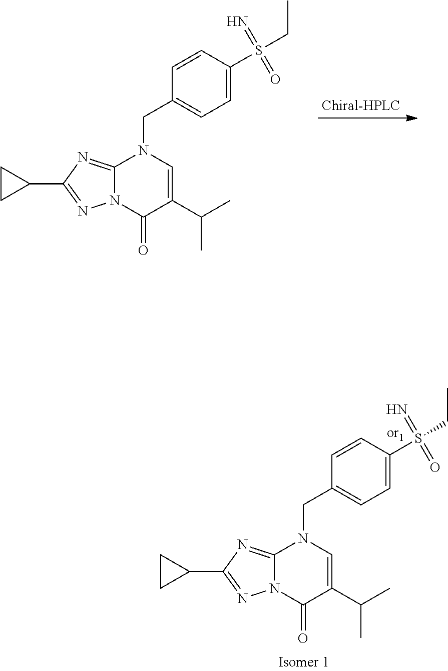 [SMILES: CCS(=N)(=O)c1ccc(Cn2cc(C(C)C)c(=O)n3nc(C4CC4)nc23)cc1 ; CC[S@](=N)(=O)c1ccc(Cn2cc(C(C)C)c(=O)n3nc(C4CC4)nc23)cc1]

The racemate (100 mg) was separated by Chiral-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 202/220 nm; RT1 (min): 36.449; Sample Solvent: ETOH: DCM=1:1; Number Of Runs: 7), and the pure fraction was concentrated and then lyophilized to afford rel-(S)-2-cyclopropyl-4-(4-(ethylsulfonimidoyl)benzyl)-6-isopropyl-[1,2,4]triazolo[1,5-a]pyrimidin-7(4H)-one.

ESI-MS m/z=400.15 [M+H]⁺; Calculated: 399.2.

¹H NMR (400 MHZ, DMSO-d6) δ 8.03 (s, 1H), 7.89-7.80 (m, 2H), 7.60-7.50 (m, 2H), 5.42 (s, 2H), 4.19 (s, 1H), 3.10 (q, J=7.3 Hz, 2H), 3.03-2.90 (m, 1H), 2.11-1.96 (m, 1H), 1.19 (d, J=6.9 Hz, 6H), 1.07-0.96 (m, 5H), 0.91-0.83 (m, 2H).

### Compound 57: 2-Cyclopropyl-6-isopropyl-4-(4-(S-methylsulfonimidoyl)benzyl)pyrazolo[1,5-a]pyrimidin-7(4H)-one

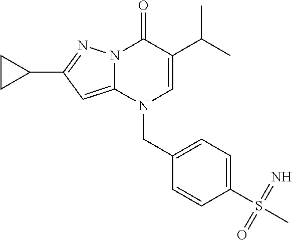 [SMILES: CC(C)c1cn(Cc2ccc(S(C)(=N)=O)cc2)c2cc(C3CC3)nn2c1=O]

### Step 1: 2-Cyclopropyl-6-isopropylpyrazolo[1,5-a]pyrimidin-7(4H)-one

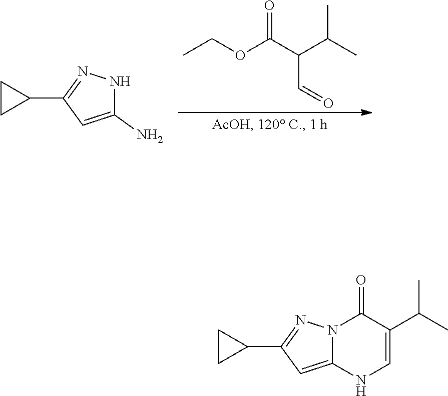 [SMILES: Nc1cc(C2CC2)n[nH]1 ; CCOC(=O)C(C=O)C(C)C ; CC(C)c1c[nH]c2cc(C3CC3)nn2c1=O]

A mixture of ethyl 2-formyl-3-methylbutanoate (500 mg, 3.16 mmol, 1.0 equiv) and 5-cyclopropyl-2H-pyrazol-3-amine (389 mg, 3.16 mmol, 1.0 equiv) in AcOH (4 mL) was stirred for 1 h at 120° C. under poured into water (30 mL). The resulting mixture was extracted with EtOAc (3×30 mL). The combined organic layers were washed with brine (15 mL), and dried over anhydrous Na₂SO₄. After filtration, the filtrate was concentrated under reduced pressure. The residue was purified by reversed-phase flash chromatography with the following conditions: column, C18; mobile phase, MeCN in Water (10 mmol/L NH₄HCO₃), 15% to 45% gradient in 20 min; detector, UV 220 nm to afford 2-cyclopropyl-6-isopropylpyrazolo[1,5-a]pyrimidin-7(4H)-one.

ESI-MS m/z=218.15 [M+H]⁺; Calculated: 217.1.

¹H NMR (400 MHZ, DMSO-d₆) δ 7.57 (s, 1H), 5.81 (s, 1H), 3.05-2.90 (m, 1H), 2.03-1.89 (m, 1H), 1.28-1.13 (m, 7H), 0.99-0.88 (m, 2H), 0.81-0.73 (m, 2H).

### Step 2: 2-Cyclopropyl-6-isopropyl-4-(4-(S-methylsulfonimidoyl)benzyl)pyrazolo[1,5-a]pyrimidin-7(4H)-one

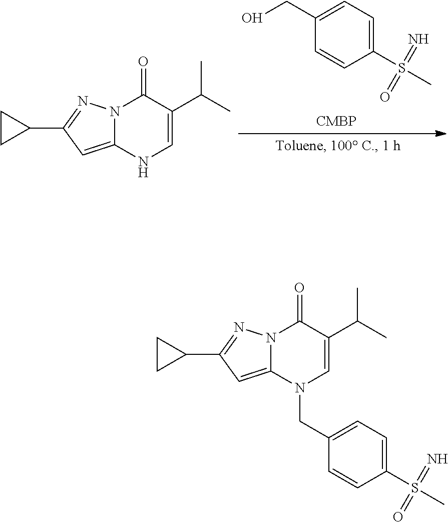 [SMILES: CC(C)c1c[nH]c2cc(C3CC3)nn2c1=O ; CS(=N)(=O)c1ccc(CO)cc1 ; CC(C)c1cn(Cc2ccc(S(C)(=N)=O)cc2)c2cc(C3CC3)nn2c1=O]

To a stirred mixture of 2-cyclopropyl-6-isopropylpyrazolo[1,5-a]pyrimidin-7(4H)-one (160 mg, 0.73 mmol, 1.0 equiv) and [4-(hydroxymethyl)phenyl](imino)methyl-lambda6-sulfanone (150 mg, 0.81 mmol, 1.1 equiv) in toluene (3 mL) was added CMBP (533 mg, 2.20 mmol, 3.0 equiv) at 25° C. under nitrogen atmosphere. The resulting mixture was stirred for 1 h at 100° C. under nitrogen atmosphere. The desired product could be detected by LCMS. The resulting mixture was concentrated under reduced pressure. The residue was purified by reversed-phase flash chromatography with the following conditions: column, C18; mobile phase, MeCN in Water (10 mmol/L NH₄HCO₃), 16% to 44% gradient in 10 min; detector, UV 220 nm to afford the crude product. The crude product was purified by Prep-HPLC with the following conditions (Column: Sunfire prep C18 column 30*150 mm, 5 m; Mobile Phase A: Water (0.1% FA), Mobile Phase B: MEOH; Flow rate: 60 mL/min mL/min; Gradient: 29% B to 49% B in 10 min; Wave Length: 254 nm/220 nm nm; RT1 (min): 9), the pure fraction was concentrated under reduced pressure then lyophilized to afford 2-cyclopropyl-6-isopropyl-4-(4-(S-methylsulfonimidoyl)benzyl)pyrazolo[1,5-a]pyrimidin-7(4H)-one.

ESI-MS m/z=385.05 [M+H]⁺; Calculated: 384.2.

¹H NMR (400 MHZ, DMSO-d₆) δ 7.98-7.88 (m, 3H), 7.56-7.50 (m, 2H), 6.03 (s, 1H), 5.33 (s, 2H), 4.21 (s, 1H), 3.04 (s, 3H), 3.05-2.94 (m, 1H), 1.99-1.88 (m, 1H), 1.23-1.17 (m, 6H), 0.98-0.87 (m, 2H), 0.78-0.70 (m, 2H).

### Compound 58 Rac: 2-Cyclopropyl-6-isopropyl-4-(3-methyl-4-(S-methylsulfonimidoyl)benzyl)pyrazolo[1,5-a]pyrimidin-7(4H)-one

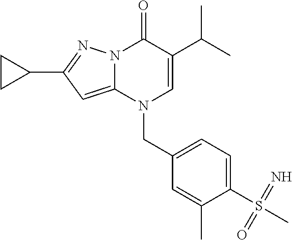 [SMILES: Cc1cc(Cn2cc(C(C)C)c(=O)n3nc(C4CC4)cc23)ccc1S(C)(=N)=O]

### Step 1: 2-Cyclopropyl-6-isopropyl-4-(3-methyl-4-(S-methylsulfonimidoyl)benzyl)pyrazolo[1,5-a]pyrimidin-7(4H)-one

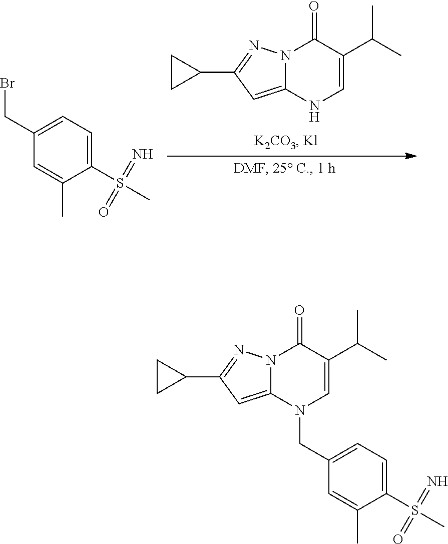 [SMILES: Cc1cc(CBr)ccc1S(C)(=N)=O ; CC(C)c1c[nH]c2cc(C3CC3)nn2c1=O ; Cc1cc(Cn2cc(C(C)C)c(=O)n3nc(C4CC4)cc23)ccc1S(C)(=N)=O]

To a stirred solution of 2-cyclopropyl-6-isopropyl-4H-pyrazolo[1,5-a]pyrimidin-7-one (300 mg, 1.38 mmol, 1.0 equiv) and [4-(bromomethyl)-2-methylphenyl](imino)methyl-lambda6-sulfanone (434 mg, 1.66 mmol, 1.2 equiv) in DMF (6 mL) were added K₂CO₃ (382 mg, 2.76 mmol, 2.0 equiv) and KI (229 mg, 1.38 mmol, 1.0 equiv) at 25° C. The resulting mixture was stirred for 1 h at 25° C. under nitrogen atmosphere. The desired product could be detected by LCMS. The resulting mixture was diluted with water (100 mL). The resulting mixture was extracted with CH₂Cl₂/IPA=3:1 (3×100 mL). The combined organic layers were washed with brine (200 mL), and dried over anhydrous Na₂SO₄. After filtration, the filtrate was concentrated under reduced pressure. The crude product (200 mg) was purified by Prep-HPLC with the following conditions (Column: XBridge Prep Phenyl OBD Column 19*250 mm, 5 m; Mobile Phase A: Water (10 mmol/L NH₄HCO₃), Mobile Phase B: ACN; Flow rate: 60 mL/min; Gradient: 5% B to 5% B in 1 min, 5% B to 20% B in 2 min, 20% to 35% B in 10 min; Wave Length: 254 nm/220 nm nm; RT1 (min): 8.63), the pure fraction was concentrated then lyophilized to afford 2-cyclopropyl-6-isopropyl-4-(3-methyl-4-(S-methylsulfonimidoyl)benzyl)pyrazolo[1,5-a]pyrimidin-7(4H)-one.

ESI-MS m/z=399.10 [M+H]⁺; Calculated: 398.2.

¹H NMR (400 MHZ, DMSO-d₆) δ 7.97-7.89 (m, 2H), 7.37-7.33 (m, 1H), 7.33-7.27 (m, 1H), 6.02 (br, 1H), 5.25 (s, 2H), 4.27 (s, 1H), 3.08-3.03 (m, 3H), 3.03-2.96 (br, 1H), 2.65 (s, 3H), 1.97-1.89 (m, 1H), 1.24-1.16 (m, 6H), 0.97-0.90 (m, 2H), 0.77-0.71 (m, 2H).

### Compound 58A: Rel-(S)-2-cyclopropyl-6-isopropyl-4-(3-methyl-4-(S-methylsulfonimidoyl)benzyl)pyrazolo[1,5-a]pyrimidin-7(4H)-one

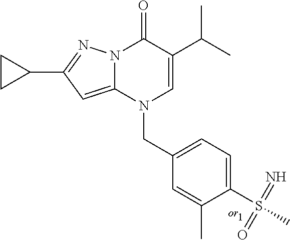 [SMILES: Cc1cc(Cn2cc(C(C)C)c(=O)n3nc(C4CC4)cc23)ccc1[S@@](C)(=N)=O]

### Step 1: Rel-(S)-2-Cyclopropyl-6-isopropyl-4-(3-methyl-4-(S-methylsulfonimidoyl)benzyl)pyrazolo[1,5-a]pyrimidin-7(4H)-one

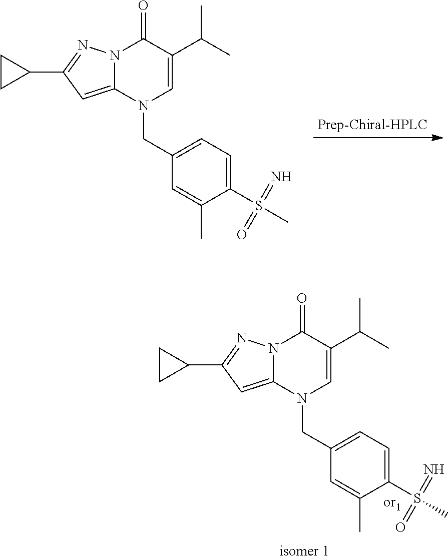 [SMILES: Cc1cc(Cn2cc(C(C)C)c(=O)n3nc(C4CC4)cc23)ccc1S(C)(=N)=O ; Cc1cc(Cn2cc(C(C)C)c(=O)n3nc(C4CC4)cc23)ccc1[S@@](C)(=N)=O]

The racemate (50 mg) was separated by PREP-CHIRAL-HPLC with the following conditions (Column: CHIRALPAK-IK, 3*25 mm, 5 μm; Mobile Phase A: Hex (10 mM NH₃-MeOH), Mobile Phase B: EtOH-HPLC; Flow rate: 40 mL/min; Gradient: isocratic 50; Wave Length: 200/210 nm; RT1 (min): 16.8; Sample Solvent: EtOH-HPLC; Injection Volume: 1.5 mL; Number Of Runs: 3), the pure factions were concentrated under vacuum then lyophilized to afford rel-(S)-2-cyclopropyl-6-isopropyl-4-(3-methyl-4-(S-methylsulfonimidoyl)benzyl)pyrazolo[1,5-a]pyrimidin-7(4H)-one.

ESI-MS m/z=399.15 [M+H]⁺; Calculated: 398.2.

¹H NMR (400 MHZ, DMSO-d₆) δ 7.97-7.90 (m, 2H), 7.38-7.26 (m, 2H), 6.03 (br, 1H), 5.25 (s, 2H), 4.27 (s, 1H), 3.07-2.96 (m, 4H), 2.65 (s, 3H), 1.97-1.89 (m, 1H), 1.25-1.14 (m, 6H), 0.97-0.90 (m, 2H), 0.78-0.71 (m, 2H).

### Compound 59 Rac: 2-Cyclopropyl-6-isopropyl-4-(4-(S-methylsulfonimidoyl)-3-(trifluoromethyl)benzyl)-[1,2,4]triazolo[1,5-a]pyrimidin-7(4H)-one

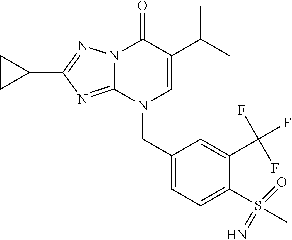 [SMILES: CC(C)c1cn(Cc2ccc(S(C)(=N)=O)c(C(F)(F)F)c2)c2nc(C3CC3)nn2c1=O]

### Step 1: 2-Cyclopropyl-6-isopropyl-4-(4-(S-methylsulfonimidoyl)-3-(trifluoromethyl)benzyl)-[1,2,4]triazolo[1,5-a]pyrimidin-7(4H)-one

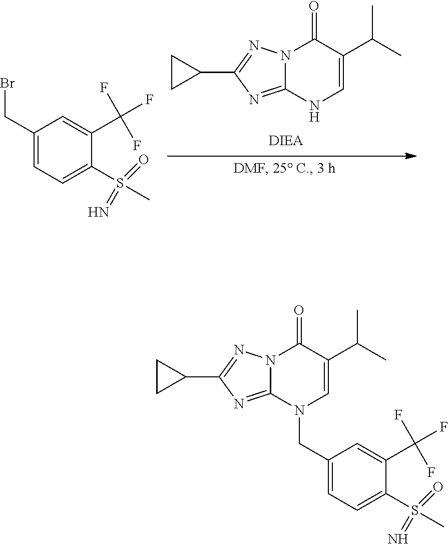

To a solution of 2-cyclopropyl-6-isopropyl-4H-[1,2,4]triazolo[1,5-a]pyrimidin-7-one (300 mg, 1.38 mmol, 1.0 equiv) and [4-(hydroxymethyl)-2-(trifluoromethyl)phenyl](imino)methyl-lambda6-sulfanone (316 mg, 1.79 mmol, 1.3 equiv) in DMF (3 mL) was added DIEA (533 mg, 4.13 mmol, 3.0 equiv) at 25° C. under nitrogen atmosphere. The resulting mixture was stirred for 3 h at 25° C. under nitrogen atmosphere. The reaction was monitored by LCMS. The residue was purified by Prep-HPLC with the following conditions (Column: XSelect CSH Fluoro Phenyl 30*150 mm, 5 m; Mobile Phase A: Water (0.1% FA), Mobile Phase B: ACN; Flow rate: 60 mL/min mL/min; Gradient: 20% B to 40% B in 9 min; Wave Length: 254 nm/220 nm nm; RT1 (min): 9.02), the pure fraction was concentrated then lyophilized to afford 2-cyclopropyl-6-isopropyl-4-(4-(S-methylsulfonimidoyl)-3-(trifluoromethyl)benzyl)-[1,2,4]triazolo[1,5-a]pyrimidin-7(4H)-one.

ESI-MS m/z=454.10 [M+H]⁺; Calculated: 453.1.

¹H NMR (400 MHZ, DMSO-d₆) δ 8.24 (d, J=8.2 Hz, 1H), 8.11 (d, J=6.5 Hz, 2H), 7.86 (d, J=8.2 Hz, 1H), 5.47 (s, 2H), 4.64-4.52 (m, 1H), 3.11 (s, 3H), 3.00-2.96 (m, 1H), 2.04 (tt, J=8.8, 4.9 Hz, 1H), 1.19 (d, J=6.8 Hz, 6H), 1.04-0.98 (m, 2H), 0.92-0.86 (m, 2H).

¹⁹F NMR (377 MHz, DMSO) δ −55.05.

### Compound 59A: Rel-(R)-2-cyclopropyl-6-isopropyl-4-(4-(S-methylsulfonimidoyl)-3-(trifluoromethyl)benzyl)-[1,2,4]triazolo[1,5-a]pyrimidin-7(4H)-one

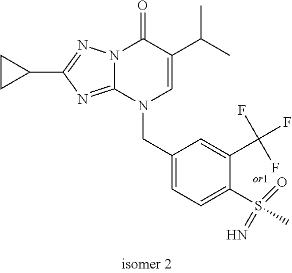 [SMILES: CC(C)c1cn(Cc2ccc([S@](C)(=N)=O)c(C(F)(F)F)c2)c2nc(C3CC3)nn2c1=O]

### Step 1: Rel-(R)-2-cyclopropyl-6-isopropyl-4-(4-(S-methylsulfonimidoyl)-3-(trifluoromethyl)benzyl)-[1,2,4]triazolo[1,5-a]pyrimidin-7(4H)-one

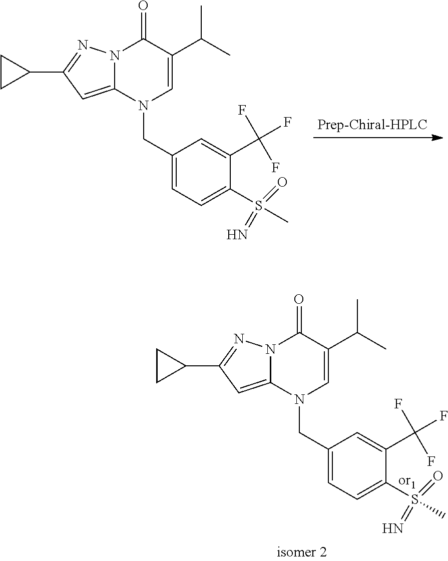 [SMILES: CC(C)c1cn(Cc2ccc(S(C)(=N)=O)c(C(F)(F)F)c2)c2cc(C3CC3)nn2c1=O ; CC(C)c1cn(Cc2ccc([S@](C)(=N)=O)c(C(F)(F)F)c2)c2cc(C3CC3)nn2c1=O]

The racemate (100 mg) was separated by Prep-Chiral-HPLC with the following conditions (Column: CHIRALPAK IH 3*25 cm, 5 μm; Mobile Phase A: Hex (10 mM NH₃-MeOH), Mobile Phase B: EtOH-HPLC; Flow rate: 40 mL/min; Gradient: isocratic 30; Wave Length: 220/280 nm; RT2 (min): 31.017; Sample Solvent: EtOH-HPLC; Injection Volume: 1 mL; Number Of Runs: 2), the pure factions were concentrated under vacuum then lyophilized to afford rel-(R)-2-cyclopropyl-6-isopropyl-4-(4-(S-methylsulfonimidoyl)-3-(trifluoromethyl)benzyl)-[1,2,4]triazolo[1,5-a]pyrimidin-7(4H)-one.

ESI-MS m/z=454.05 [M+H]⁺; Calculated: 453.1.

¹H NMR (400 MHZ, DMSO-d₆) δ 8.24 (d, J=8.2 Hz, 1H), 8.13-8.08 (m, 2H), 7.85 (dd, J=8.1, 1.8 Hz, 1H), 5.46 (s, 2H), 4.58 (d, J=1.8 Hz, 1H), 3.10 (d, J=1.5 Hz, 3H), 2.99 (p, J=6.9 Hz, 1H), 2.04 (tt, J=8.2, 4.8 Hz, 1H), 1.19 (d, J=6.9 Hz, 6H), 1.01 (dq, J=6.1, 3.6 Hz, 2H), 0.91-0.84 (m, 2H).

¹⁹F NMR (377 MHz, DMSO) δ −55.05.

### Compound 60: Rel-(R)-2-cyclopropyl-6-isopropyl-4-(4-(S-(methyl-d₃) sulfonimidoyl)benzyl)-[1,2,4]triazolo[1,5-a]pyrimidin-7(4H)-one

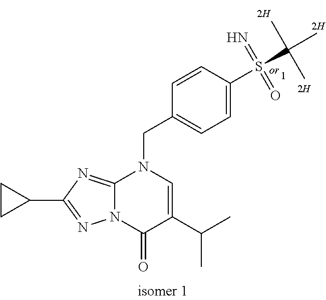 [SMILES: CC(C)c1cn(Cc2ccc([S@](=N)(=O)C(C)(C)C)cc2)c2nc(C3CC3)nn2c1=O]

### Step 1: 4-[(2H3)methylsulfanyl]benzoic acid

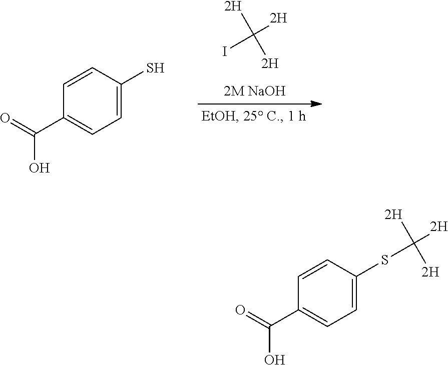

To a stirred mixture of 2-mercaptobenzoic acid (10.00 g, 64.8 mmol, 1.0 equiv) in EtOH (200 mL) were added iodomethane-d₃ (18.80 g, 129.7 mmol, 2.0 equiv) and NaOH (65 mL, 130.0 mmol, 2.0 equiv, 2M in water) dropwise at 25° C. under nitrogen atmosphere. The resulting mixture was stirred at 25° C. for 1 h under nitrogen atmosphere. The resulting mixture was diluted with water (1 L). The residue was acidified to pH 3 with 1M HCl (aq.). The precipitated solids were collected by filtration and washed with water (3×50 mL). The solids were concentrated under vacuum to afford 4-[(2H3)methylsulfanyl]benzoic acid.

ESI-MS m/z=212.95 [M+H+41]⁺; Calculated: 171.0.

¹H NMR (400 MHZ, DMSO-d₆) δ 12.83 (s, 1H), 7.85 (d, J=8.6 Hz, 2H), 7.33 (d, J=8.5 Hz, 2H).

### Step 2: {4-[(2H3)methylsulfanyl]phenyl}methanol

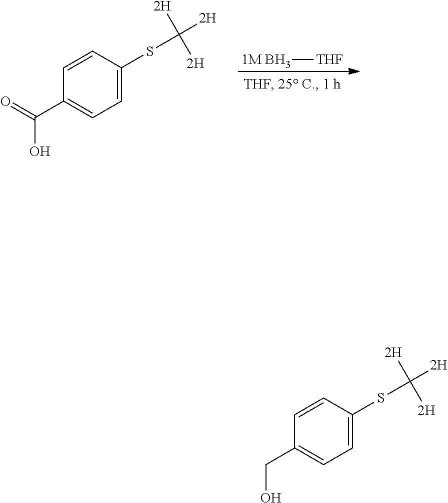

To a stirred solution of 4-[(2H3)methylsulfanyl]benzoic acid (11.00 g, 64.2 mmol, 1.0 equiv) in THF (130 mL) was added borane-tetrahydrofuran complex (129 mL, 129.0 mmol, 2.0 equiv, 1.0 M in THF) dropwise at 10° C. under nitrogen atmosphere. The desired product could be detected by LCMS. The reaction was poured into water (750 mL) at 0° C. The resulting mixture was added brine (250 mL), then extracted with EtOAc (2×1 L). The combined organic layers were washed with brine (1×1 L), and dried over anhydrous Na₂SO₄. After filtration, the filtrate was concentrated under reduced pressure. The residue was purified by silica gel column chromatography, eluted with EA in PE (10% to 40% gradient in 20 min, hold at 27%) to afford {4-[(2H3)methylsulfanyl]phenyl}methanol.

ESI-MS m/z=156.15 [M−H]⁻; Calculated: 157.1.

¹H NMR (400 MHZ, DMSO-d₆) δ 7.30-7.18 (m, 4H), 5.48-4.64 (m, 1H), 4.44 (s, 2H).

### Step 3: 1-(Chloromethyl)-4-[(2H3)methylsulfanyl]benzene

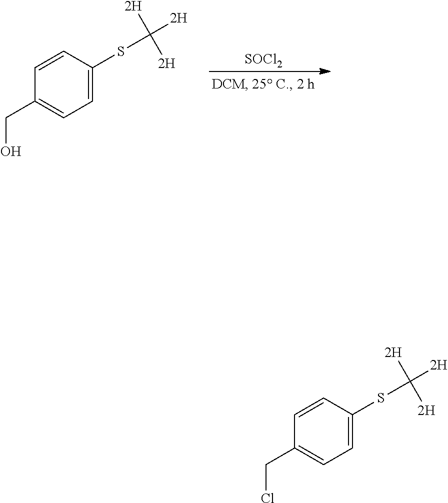

To a stirred mixture of {4-[(2H3)methylsulfanyl]phenyl}methanol (500 mg, 3.18 mmol, 1.0 equiv) in DCM (10 mL) was added thionyl chloride (757 mg, 6.36 mmol, 2.0 equiv) dropwise at 25° C. under nitrogen atmosphere. The resulting mixture was stirred at 25° C. for 2 h under nitrogen atmosphere. The desired product could be detected by LCMS. The resulting mixture was concentrated under reduced pressure to afford 1-(chloromethyl)-4-[(2H3)methylsulfanyl]benzene (crude).

ESI-MS m/z=140.20 [M-Cl]⁺; Calculated: 175.0.

### Step 4: 2-Cyclopropyl-6-isopropyl-4-({4-[(2H3)methylsulfanyl]phenyl}methyl)-[1,2,4]triazolo[1,5-a]pyrimidin-7-one

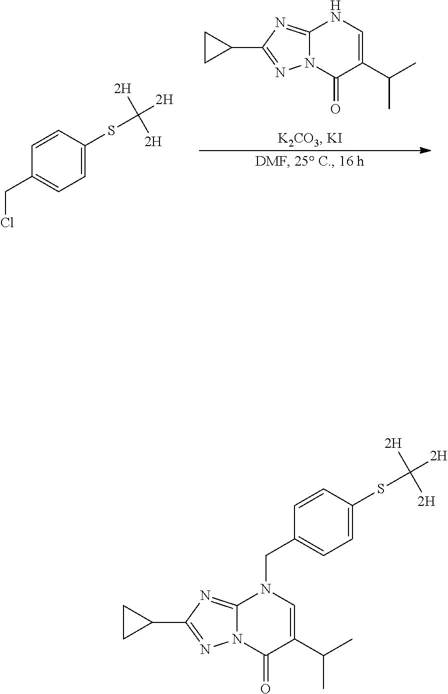

To a stirred mixture of 2-cyclopropyl-6-isopropyl-4H-[1,2,4]triazolo[1,5-a]pyrimidin-7-one (745 mg, 3.41 mmol, 1.0 equiv) and K₂CO₃ (1.42 g, 10.2 mmol, 3.0 equiv) in DMF (10 mL) were added KI (567 mg, 3.41 mmol, 1.0 equiv) and 1-(chloromethyl)-4-[(2H3)methylsulfanyl]benzene (600 mg, crude) at 25° C. under nitrogen atmosphere. The resulting mixture was stirred at 25° C. for 16 h under nitrogen atmosphere. The desired product could be detected by LCMS. The resulting mixture was diluted with water (100 mL). The resulting mixture was extracted with EtOAc (3×100 mL). The combined organic layers were washed with brine (1×200 mL), and dried over anhydrous Na₂SO₄. After filtration, the filtrate was concentrated under reduced pressure. The residue was purified by silica gel column chromatography, eluted with EA in PE (10% to 50% gradient in 30 min, hold at 43%) to afford 2-cyclopropyl-6-isopropyl-4-({4-[(2H3)methylsulfanyl]phenyl}methyl)-[1,2,4]triazolo[1,5-a]pyrimidin-7-one.

ESI-MS m/z=358.10 [M+H]⁺; Calculated: 357.2.

¹H NMR (400 MHZ, Methanol-d₄) δ 7.75 (d, J=0.8 Hz, 1H), 7.36-7.28 (m, 2H), 7.27-7.20 (m, 2H), 5.30 (s, 2H), 3.12-3.01 (m, 1H), 2.14-2.05 (m, 1H), 1.23 (d, J=6.9 Hz, 6H), 1.09-1.03 (m, 4H).

### Step 5: 2-Cyclopropyl-4-({4-[imino ((2H3)methyl) oxo-lambda6-sulfanyl]phenyl}methyl)-6-isopropyl-[1,2,4]triazolo[1,5-a]pyrimidin-7-one

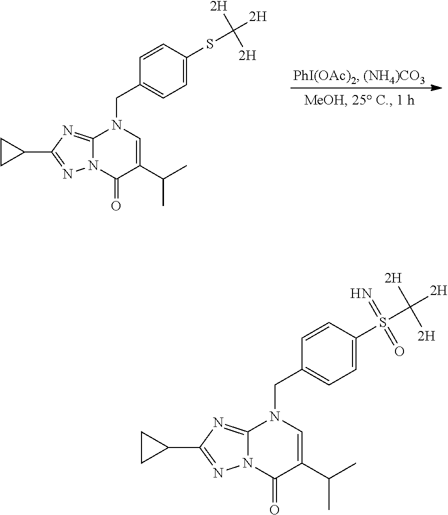

A mixture of 2-cyclopropyl-6-isopropyl-4-({4-[(2H3)methylsulfanyl]phenyl}methyl)-[1,2,4]triazolo[1,5-a]pyrimidin-7-one (500 mg, 1.40 mmol, 1.0 equiv) and (diacetoxyiodo)benzene (1.58 g, 4.90 mmol, 3.5 equiv) in methan-d₃-ol-d (10 mL) was stirred at 25° C. for 10 min under nitrogen atmosphere. To the above mixture was added ammonium carbonate (309 mg, 3.22 mmol, 2.3 equiv) at 25° C. The resulting mixture was stirred at 25° C. for an additional 1 h. The desired product could be detected by LCMS. The resulting mixture was concentrated under reduced pressure. The resulting mixture was diluted with acetonitrile (100 mL). The resulting mixture was filtered; the filter cake was washed with acetonitrile (3×50 mL). The filtrate was concentrated under reduced pressure. The residue was purified by reverse phase flash with the following conditions (column, C18; mobile phase, MeCN in Water (10 mmol/L NH₄HCO₃), 20% to 80% gradient in 20 min, hold at 38%; detector, UV 254/220 nm) to afford the crude product. The crude product was purified by Prep-HPLC with the following conditions (Column: XBridge Prep OBD C18 Column 30*150 mm, 5 μm; Mobile Phase A: 10 mmol/L NH₄HCO₃+0.05% NH₃H₂O), Mobile Phase B: ACN; Flow rate: 60 mL/min; Gradient: 5% B to 5% B in 1 min, 5% B to 13% B in 2 min, 13% to 30% B in 11 min; Wave Length: 254 nm/220 nm; RT1 (min): 10.23) to afford 2-cyclopropyl-4-({4-[imino ((2H3)methyl) oxo-lambda6-sulfanyl]phenyl}methyl)-6-isopropyl-[1,2,4]triazolo[1,5-a]pyrimidin-7-one.

ESI-MS m/z=389.20 [M+H]⁺; Calculated: 388.2.

¹H NMR (400 MHZ, DMSO-d₆) δ 8.03 (s, 1H), 7.94-7.86 (m, 2H), 7.61-7.51 (m, 2H), 5.41 (s, 2H), 4.19 (s, 1H), 3.04-2.93 (m, 1H), 2.07-1.99 (m, 1H), 1.18 (d, J=6.9 Hz, 6H), 1.04-0.95 (m, 2H), 0.93-0.85 (m, 2H).

Step 6: Rel-(R)-2-cyclopropyl-6-isopropyl-4-(4-(S-(methyl-d₃) sulfonimidoyl)benzyl)-[1,2,4]triazolo[1,5-a]pyrimidin-7-one

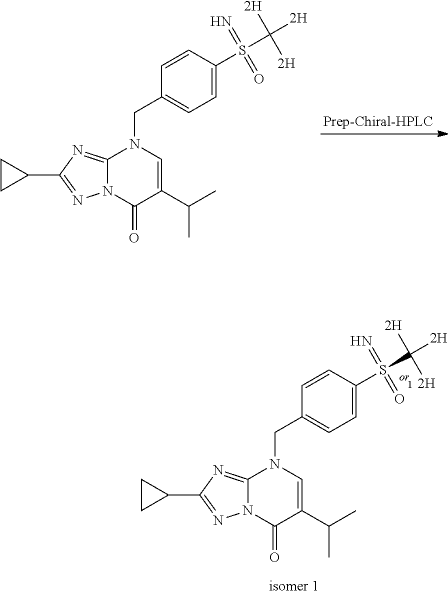

The 2-cyclopropyl-4-({4-[imino ((2H3)methyl) oxo-lambda6-sulfanyl]phenyl}methyl)-6-isopropyl-[1,2,4]triazolo[1,5-a]pyrimidin-7-one (100 mg, 0.26 mmol, 1.0 equiv) was separated by PREP-CHIRAL-HPLC with the following conditions (Column: CHIRALPAK-IK, 3*25 mm, 5 μm; Mobile Phase A: Hex (0.1% DEA)-HPLC, Mobile Phase B: EtOH; Flow rate: 40 mL/min; Gradient: isocratic 50; Wave Length: 218/284 nm; RT1 (min): 16.712; RT2 (min): 21.662; Sample Solvent: ETOH: DCM=1:1; Injection Volume: 0.6 mL; Number Of Runs: 5), the pure factions were concentrated under vacuum then lyophilized to afford rel-(R)-2-cyclopropyl-6-isopropyl-4-(4-(S-(methyl-d₃) sulfonimidoyl)benzyl)-[1,2,4]triazolo[1,5-a]pyrimidin-7(4H)-one.

ESI-MS m/z=389.15 [M+H]⁺; Calculated: 388.2.

¹H NMR (400 MHZ, DMSO-d₆) δ 8.04 (s, 1H), 7.95-7.85 (m, 2H), 7.61-7.50 (m, 2H), 5.41 (s, 2H), 4.19 (s, 1H), 3.06-2.91 (m, 1H), 2.09-1.98 (m, 1H), 1.19 (d, J=6.9 Hz, 6H), 1.04-0.96 (m, 2H), 0.93-0.86 (m, 2H).

### Compound 61: 2-Cyclopropyl-6-isopropyl-4-(2-methyl-4-(S-methylsulfonimidoyl)benzyl)pyrazolo[1,5-a]pyrimidin-7(4H)-one

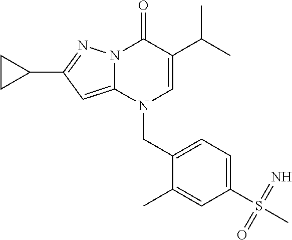 [SMILES: Cc1cc(S(C)(=N)=O)ccc1Cn1cc(C(C)C)c(=O)n2nc(C3CC3)cc12]

### Step 1: 2-Methyl-4-(methylthio)benzonitrile

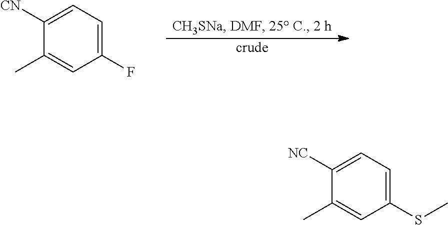

A solution of 4-fluoro-2-methylbenzonitrile (5.00 g, 36.9 mmol, 1.0 equiv) and CH₃SNa (2.85 g, 40.6 mmol, 1.1 equiv) in DMF (50 mL) was stirred for 2 h at 25° C. under nitrogen atmosphere. The reaction was monitored by TLC (PE/EA=5:1 Rf=0.2). The product was precipitated by the addition of water. The precipitated solids were collected by filtration and washed with water (3×30 mL). This resulted in 2-methyl-4-(methylthio)benzonitrile (crude).

¹H NMR (400 MHZ, DMSO-d₆) δ 7.64 (s, 1H), 7.32 (d, J=1.9 Hz, 1H), 7.21 (dd, J=8.2, 2.0 Hz, 1H), 2.52 (s, 3H), 2.45 (s, 3H).

### Step 2: 2-Methyl-4-(methylthio)benzoic acid

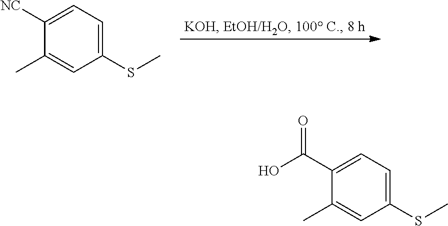

A solution of 2-methyl-4-(methylsulfanyl)benzonitrile (4.20 g, 25.7 mmol, 1.0 equiv) and KOH (11.55 g, 205.8 mmol, 8.0 equiv) in H₂O (20 mL) and EtOH (20 mL) was stirred for 8 h at 100° C. under nitrogen atmosphere. The desired product could be detected by LCMS. The mixture was allowed to cool down to 25° C. The mixture was acidified to pH 5 with 1M HCl aq. The precipitated solids were collected by filtration and washed with H₂O (10 mL). The residue was purified by silica gel column chromatography, eluted with PE/EA (10:1-5:1) to afford 2-methyl-4-(methylthio)benzoic acid.

ESI-MS m/z=181.2 [M−H]; Calculated: 182.2.

### Step 3: (2-Methyl-4-(methylthio)phenyl)methanol

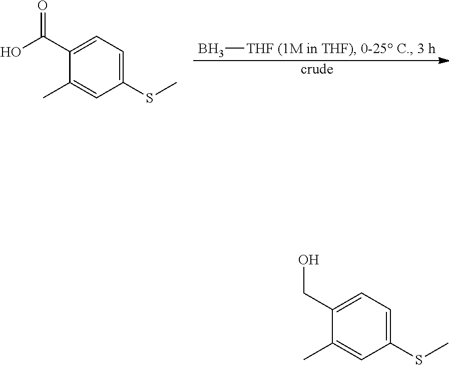

To a stirred solution of 2-methyl-4-(methylthio)benzoic acid (1.50 g, 8.20 mmol, 1.0 equiv) was added BH₃-THF (15 mL) at 0° C. The mixture was stirred for 2 h at 25° C. under nitrogen atmosphere. The desired product could be detected by LCMS. The reaction was quenched by the addition of H₂O (10 mL) at 0° C. The resulting mixture was extracted with EtOAc (3×10 mL). The combined organic layers were washed with brine (20 mL), and dried over anhydrous Na₂SO₄. After filtration, the filtrate was concentrated under reduced pressure. The resulting mixture was concentrated under vacuum. This resulted in (2-methyl-4-(methylthio)phenyl)methanol (crude).

ESI-MS m/z=151.1 [M-17]⁺; Calculated: 168.1.

### Step 4: (4-(Hydroxymethyl)-3-methylphenyl)(imino)(methyl)-l6-sulfanone

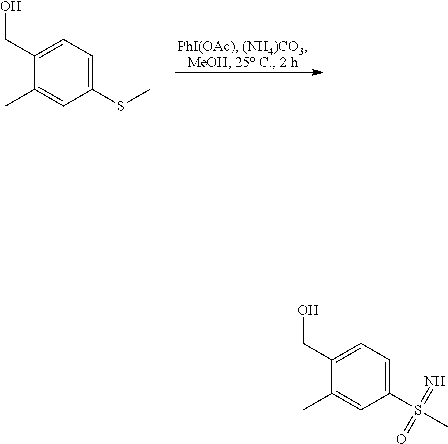

A solution of [2-methyl-4-(methylsulfanyl)phenyl]methanol (1.40 g, 8.30 mmol, 1.0 equiv), (NH₄)₂CO₃ (1.20 g, 12.5 mmol, 1.5 equiv) and PhIOAc (6.16 g, 19.1 mmol, 2.3 equiv) in MeOH (20 mL) was stirred for 2 h at 25° C. under nitrogen atmosphere. The desired product could be detected by LCMS. The resulting mixture was concentrated under reduced pressure. The residue was purified by silica gel column chromatography, eluted with CH₂Cl₂/MeOH (10:1-5:1) to afford (4-(hydroxymethyl)-3-methylphenyl)(imino)(methyl)-l6-sulfanone.

ESI-MS m/z=200.1 [M+H]⁺; Calculated: 199.1.

¹H NMR (400 MHZ, DMSO-d₆) δ 7.72 (d, J=2.0 Hz, 1H), 7.69 (d, J=2.0 Hz, 1H), 7.58 (d, J=8.0 Hz, 1H), 5.32 (t, J=5.4 Hz, 1H), 4.56 (d, J=5.3 Hz, 2H), 4.08 (s, 1H), 3.02 (d, J=1.0 Hz, 3H), 2.30 (s, 3H).

### Step 5: (4-(Bromomethyl)-3-methylphenyl)(imino)(methyl)-l6-sulfanone

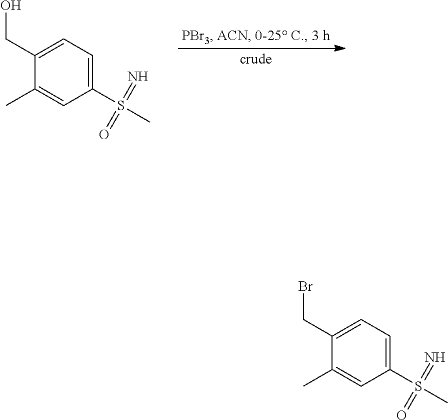

A solution of (2-methyl-4-(methylthio)phenyl)methanol (1.40 g, 7.00 mmol, 1.0 equiv) in CH₃CN (10 mL) was stirred for 3 h at 25° C. under nitrogen atmosphere followed by the addition of PBr₃ (2.85 g, 10.5 mmol, 1.5 equiv) dropwise at 0° C. The desired product could be detected by LCMS. The resulting mixture was concentrated under reduced pressure. The residue was purified by trituration with ethoxyethane (3×10 mL).

This afforded (4-(bromomethyl)-3-methylphenyl)(imino)(methyl)-l6-sulfanone (crude).

ESI-MS m/z=262.1 [M+H]⁺; Calculated: 261.0.

### Step 6: 2-Cyclopropyl-6-isopropyl-4-(2-methyl-4-(S-methylsulfonimidoyl)benzyl)pyrazolo[1,5-a]pyrimidin-7(4H)-one

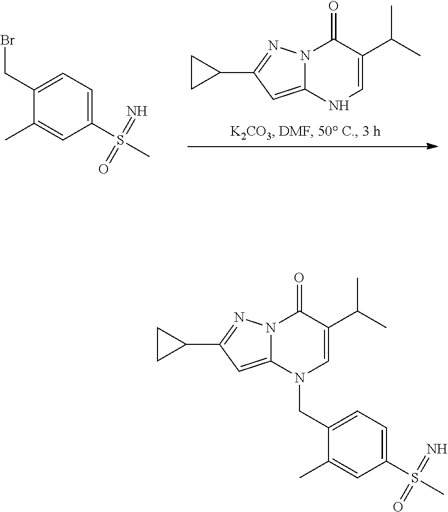 [SMILES: Cc1cc(S(C)(=N)=O)ccc1CBr ; CC(C)c1c[nH]c2cc(C3CC3)nn2c1=O ; Cc1cc(S(C)(=N)=O)ccc1Cn1cc(C(C)C)c(=O)n2nc(C3CC3)cc12]

A solution of (4-(bromomethyl)-3-methylphenyl)(imino)(methyl)-l6-sulfanone (904 mg, 3.4 mmol, 1.5 equiv) and K₂CO₃ (954 mg, 6.9 mmol, 3.0 equiv) in DMF (5 mL) was stirred for 3 h at 50° C. under nitrogen atmosphere. The desired product could be detected by LCMS. The mixture was allowed to cool down to 25° C. The resulting mixture was diluted with EA (10 mL). The resulting mixture was washed with 5×10 ml of water, and dried over anhydrous Na₂SO₄. After filtration, the filtrate was concentrated under reduced pressure. The residue was purified by Prep-HPLC with the following conditions: (Column: XBridge Prep Phenyl OBD Column 19*250 mm, 5 m; Mobile Phase A: Water (10 mmol/L NH₄HCO₃), Mobile Phase B: ACN; Flow rate: 60 mL/min mL/min; Gradient: 5% B to 5% B in 1 min, 5% B to 20% B in 2 min, 20% to 35% B in 10 min; Wave Length: 254 nm/220 nm nm; RT1 (min): 9), the pure fraction was concentrated then lyophilized to afford 2-cyclopropyl-6-isopropyl-4-(2-methyl-4-(S-methylsulfonimidoyl)benzyl)pyrazolo[1,5-a]pyrimidin-7(4H)-one.

ESI-MS m/z=399.10 [M+H]⁺; Calculated: 398.2.

¹H NMR (400 MHZ, DMSO-d₆) δ 7.81 (d, J=2.0 Hz, 1H), 7.76 (s, 1H), 7.67 (dd, J=8.1, 2.0 Hz, 1H), 6.86 (d, J=8.1 Hz, 1H), 5.92 (s, 1H), 5.32 (s, 2H), 4.15 (s, 1H), 3.05-3.01 (m, 4H), 2.43 (s, 3H), 1.93 (t, J=4.6 Hz, 1H), 1.17 (d, J=6.9 Hz, 6H), 0.92 (dt, J=8.5, 3.2 Hz, 2H), 0.73 (dd, J=4.9, 2.3 Hz, 2H).

### Compound 62: 2-Cyclopropyl-4-(2-fluoro-4-(S-methylsulfonimidoyl)benzyl)-6-isopropylpyrazolo[1,5-a]pyrimidin-7(4H)-one

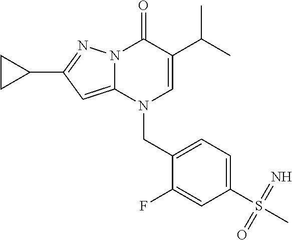 [SMILES: CC(C)c1cn(Cc2ccc(S(C)(=N)=O)cc2F)c2cc(C3CC3)nn2c1=O]

### Step 1: 2-Cyclopropyl-4-(2-fluoro-4-(S-methylsulfonimidoyl)benzyl)-6-isopropylpyrazolo[1,5-a]pyrimidin-7(4H)-one

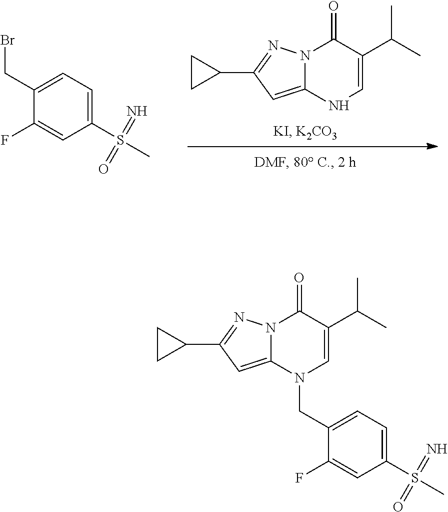 [SMILES: CS(=N)(=O)c1ccc(CBr)c(F)c1 ; CC(C)c1c[nH]c2cc(C3CC3)nn2c1=O ; CC(C)c1cn(Cc2ccc(S(C)(=N)=O)cc2F)c2cc(C3CC3)nn2c1=O]

To a stirred solution of [4-(bromomethyl)-3-fluorophenyl](imino)methyl-lambda6-sulfanone (500 mg, 1.88 mmol, 1.0 equiv) and 2-cyclopropyl-6-isopropyl-4H-pyrazolo[1,5-a]pyrimidin-7-one (490 mg, 2.26 mmol, 1.2 equiv) in DMF (6.00 mL) was added K₂CO₃ (779 mg, 5.64 mmol, 3.0 equiv) and KI (312 mg, 1.88 mmol, 1.0 equiv) at 25° C. under nitrogen atmosphere. The resulting mixture was stirred for 2 h at 80° C. under nitrogen atmosphere. The reaction was monitored by LCMS. The mixture was allowed to cool down to 25° C. The crude product was purified by Prep-HPLC with the following conditions (Column: XBridge Prep Phenyl OBD Column 19*250 mm, 5 m; Mobile Phase A: Water (10 mmol/L NH₄HCO₃), Mobile Phase B: ACN; Flow rate: 60 mL/min; Gradient: 5% B to 5% B in 1 min, 5% B to 20% B in 2 min, 20% to 35% B in 10 min; Wave Length: 254 nm/220 nm nm; RT1 (min): 8.6), the pure fraction was concentrated and then lyophilized to afford 2-cyclopropyl-4-(2-fluoro-4-(S-methylsulfonimidoyl)benzyl)-6-isopropylpyrazolo[1,5-a]pyrimidin-7(4H)-one.

ESI-MS m/z=403.15 [M+H]⁺; Calculated: 402.2.

¹H NMR (400 MHZ, DMSO-d₆) δ 7.87 (s, 1H), 7.78-7.71 (m, 2H), 7.47 (t, J=7.6 Hz, 1H), 6.01 (s, 1H), 5.38 (s, 2H), 4.38 (s, 1H), 3.09 (d, J=1.2 Hz, 3H), 3.00 (p, J=6.9 Hz, 1H), 1.94 (td, J=8.5, 4.3 Hz, 1H), 1.18 (d, J=6.9 Hz, 6H), 0.97-0.90 (m, 2H), 0.76-0.71 (m, 2H).

¹⁹F NMR (377 MHz, DMSO) δ −114.47.

### Compound 63: 2-Cyclopropyl-4-(3-fluoro-4-(S-methylsulfonimidoyl)benzyl)-6-isopropyl-[1,2,4]triazolo[1,5-a]pyrimidin-7(4H)-one

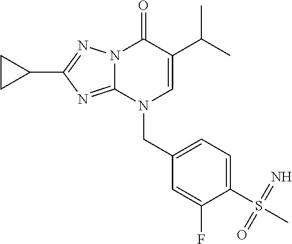 [SMILES: CC(C)c1cn(Cc2ccc(S(C)(=N)=O)c(F)c2)c2nc(C3CC3)nn2c1=O]

### Step 1: 3-Fluoro-4-(methylthio)benzonitrile

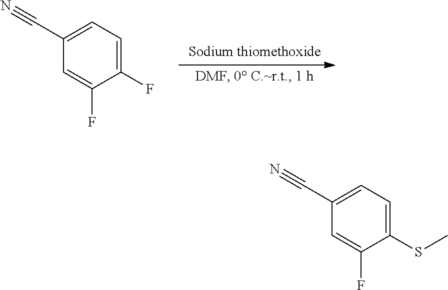

To a stirred solution of 3,4-difluorobenzonitrile (7.00 g, 50.3 mmol, 1.0 equiv) in DMF (70 mL) was added sodium thiomethoxide (3.88 g, 55.3 mmol, 1.1 equiv) at 0° C. under nitrogen atmosphere. The resulting mixture was stirred for 1 h at room temperature under nitrogen atmosphere. The desired product could be detected by LCMS (retention time: 1.082). The product was precipitated by the addition of water (700 mL). The precipitated solids were collected by filtration and washed with water (3×20 mL). This resulted in 3-fluoro-4-(methylthio)benzonitrile.

¹H NMR (400 MHZ, DMSO-d₆) δ 7.85-7.76 (m, 1H), 7.74-7.67 (m, 1H), 7.54-7.46 (m, 1H), 2.57 (s, 3H).

### Step 2: 3-Fluoro-4-(methylthio)benzoic acid

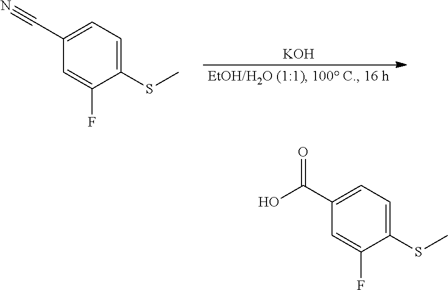

A mixture of 3-fluoro-4-(methylsulfanyl)benzonitrile (4.10 g, 24.5 mmol, 1.0 equiv) and KOH (13.42 g, 239.2 mmol, 10.0 equiv) in EtOH/H₂O (1:1, 40 mL) was stirred for 16 h at 100° C. under nitrogen atmosphere. The mixture was allowed to cool down to room temperature. The desired product could be detected by LCMS (retention time: 0.504). The mixture was acidified to pH 3 with 6 M HCl. The precipitated solids were collected by filtration and washed with water (3×8 mL) to afford 3-fluoro-4-(methylthio)benzoic acid.

¹H NMR (400 MHZ, DMSO-d₆) δ 12.62 (s, 1H), 7.81-7.74 (m, 1H), 7.67-7.58 (m, 1H), 7.48-7.39 (m, 1H), 2.55 (s, 3H).

### Step 3: (3-Fluoro-4-(methylthio)phenyl)methanol

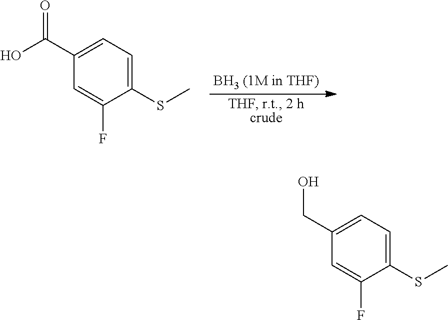

To a stirred solution of 3-fluoro-4-(methylsulfanyl)benzoic acid (2.50 g, 13.4 mmol, 1.0 equiv) in THF (10 mL) was added BH₃·THF (2.31 g, 26.8 mmol, 2.0 equiv, 1M in THF) dropwise at room temperature under nitrogen atmosphere. The resulting mixture was stirred for 2 h at room temperature under nitrogen atmosphere. The desired product could be detected by LCMS. The reaction was quenched with water/ice (100 mL) at 0° C. The aqueous layer was extracted with EtOAc (3×50 mL). The combined organic layers were dried over anhydrous Na₂SO₄. After filtration, the filtrate was concentrated under reduced pressure to afford (3-fluoro-4-(methylthio)phenyl)methanol (crude).

ESI-MS m/z=171.05 [M−H]⁻; Calculated: 172.0.

### Step 4: (2-Fluoro-4-(hydroxymethyl)phenyl)(imino)(methyl)-l6-sulfanone

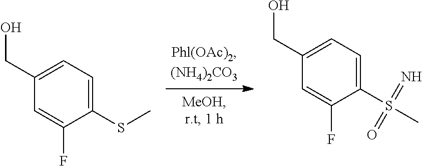

To a stirred mixture of [3-fluoro-4-(methylsulfanyl)phenyl]methanol (2.10 g, 12.1 mmol, 1.0 equiv) and PhI(OAc)₂ (9.03 g, 28.0 mmol, 2.3 equiv) in MeOH (20 mL) was added ammonium carbonate (1.76 g, 18.2 mmol, 1.5 equiv) at room temperature under nitrogen atmosphere. The resulting mixture was stirred for 1 h at room temperature under nitrogen atmosphere. The desired product could be detected by LCMS. The resulting mixture was concentrated under reduced pressure. The residue was purified by silica gel column chromatography, eluted with CH₂Cl₂/MeOH (0 to 20% in 15 min) to afford (2-fluoro-4-(hydroxymethyl)phenyl)(imino)(methyl)-l6-sulfanone.

ESI-MS m/z=203.90 [M+H]⁺; Calculated: 203.0.

¹H NMR (400 MHZ, DMSO-d₆) δ 7.86-7.78 (m, 1H), 7.35-7.33 (m, 1H), 7.33-7.29 (m, 1H), 5.51 (s, 1H), 4.58 (s, 2H), 3.16 (s, 3H).

### Step 5: (4-(Bromomethyl)-2-fluorophenyl)(imino)(methyl)-l6-sulfanone

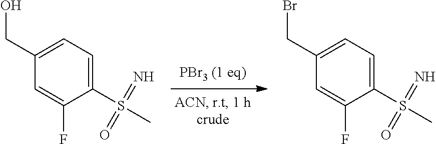

To a stirred solution of [2-fluoro-4-(hydroxymethyl)phenyl](imino)methyl-lambda6-sulfanone (2.10 g, 10.3 mmol, 1.0 equiv) in ACN (20 mL) was added PBr₃ (5.59 g, 20.6 mmol, 2.0 equiv) dropwise at room temperature under nitrogen atmosphere. The resulting mixture was stirred for 1 h at room temperature under nitrogen atmosphere. The desired product could be detected by LCMS. The resulting mixture was concentrated under reduced pressure. The residue was purified by trituration with diethyl ether (5×20 mL) to afford (4-(bromomethyl)-2-fluorophenyl)(imino)(methyl)-l6-sulfanone (crude).

ESI-MS m/z=265.80, 267.80 [M+H]⁺; Calculated: 265.0, 267.0.

### Step 6: 2-Cyclopropyl-4-(3-fluoro-4-(S-methylsulfonimidoyl)benzyl)-6-isopropyl-[1,2,4]triazolo[1,5-a]pyrimidin-7(4H)-one

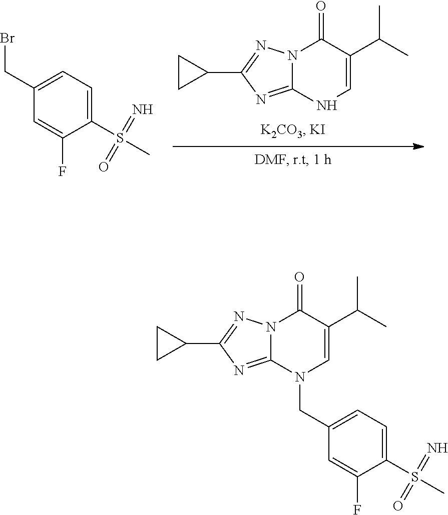

To a stirred mixture of [4-(bromomethyl)-2-fluorophenyl](imino)methyl-lambda6-sulfanone (300 mg, 1.12 mmol, 1.0 equiv) and 2-cyclopropyl-6-isopropyl-4H-[1,2,4]triazolo[1,5-a]pyrimidin-7-one (270 mg, 1.24 mmol, 1.1 equiv) in DMF (3 mL) were added K₂CO₃ (467 mg, 3.38 mmol, 3.0 equiv) and KI (187 mg, 1.12 mmol, 1.0 equiv) at room temperature under nitrogen atmosphere. The resulting mixture was stirred for 1 h at room temperature under nitrogen atmosphere. The desired product could be detected by LCMS. The resulting mixture was poured into water (30 mL). The aqueous layer was extracted with EtOAc (2×50 mL). The combined organic layers were dried over anhydrous Na₂SO₄. After filtration, the filtrate was concentrated under reduced pressure. The mixture was purified by Prep-HPLC with the following conditions (Column: YMC-Actus Triart C18 ExRS 30*150 mm, 5 m; Mobile Phase A:

Water (10 mmol/L NH₄HCO₃), Mobile Phase B: ACN; Flow rate: 60 mL/min; Gradient: 15% B to 40% B in 7 min; Wave Length: 254 nm/220 nm nm; RT1 (min): 6.4), the pure fraction was concentrated under reduced pressure then lyophilized to afford 2-cyclopropyl-4-(3-fluoro-4-(S-methylsulfonimidoyl)benzyl)-6-isopropyl-[1,2,4]triazolo[1,5-a]pyrimidin-7(4H)-one.

ESI-MS m/z=404.20 [M+H]⁺; Calculated: 403.1.

¹H NMR (400 MHZ, DMSO-d₆) δ 8.02 (s, 1H), 7.89-7.81 (m, 1H), 7.52-7.44 (m, 1H), 7.41-7.34 (m, 1H), 5.41 (s, 2H), 4.71 (s, 1H), 3.19-3.14 (m, 3H), 3.06-2.92 (m, 1H), 2.11-1.99 (m, 1H), 1.19 (d, J=6.9 Hz, 3H), 1.19 (d, J=6.9 Hz, 3H), 1.06-0.95 (m, 2H), 0.97-0.85 (m, 2H).

### Compound 64: 2-Cyclopropyl-6-isopropyl-4-(4-(propan-2-ylsulfonimidoyl)benzyl)-[1,2,4]triazolo[1,5-a]pyrimidin-7(4H)-one

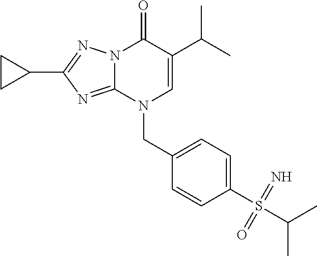 [SMILES: CC(C)c1cn(Cc2ccc(S(=N)(=O)C(C)C)cc2)c2nc(C3CC3)nn2c1=O]

### Step 1: 4-(Isopropylsulfanyl)benzoic acid

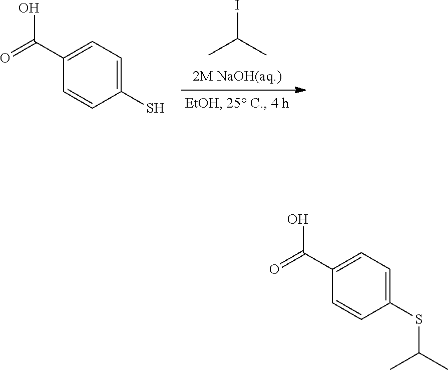

To a stirred mixture of 2-mercaptobenzoic acid (4.00 g, 25.9 mmol, 1.0 equiv) in EtOH (80 mL) were added 2-iodopropane (8.82 g, 51.9 mmol, 2.0 equiv) and 2M NaOH(aq.) (7.1 mL, 14.3 mmol, 2.0 equiv) at 25° C. The resulting mixture was stirred for 4 h at 25° C. under nitrogen atmosphere. The desired product could be detected by LCMS. The resulting mixture was diluted with water (400 mL). The mixture was acidified to pH 3 with 1M HCl (aq.). The precipitated solids were collected by filtration and washed with water (2×50 mL). The resulting mixture was concentrated under reduced pressure to afford 4-(isopropylsulfanyl)benzoic acid.

ESI-MS m/z=195.00 [M−H]⁻; Calculated: 196.1.

¹H NMR (400 MHZ, DMSO-d₆) δ 12.90 (s, 1H), 7.91-7.79 (m, 2H), 7.44-7.35 (m, 2H), 3.75-3.60 (m, 1H), 1.29 (d, J=6.6 Hz, 6H).

### Step 2: 4-(Isopropylsulfanyl)phenyl]methanol

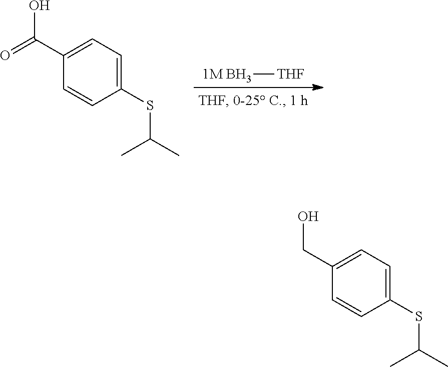

To a solution of 4-(isopropylsulfanyl)benzoic acid (5.00 g, 25.4 mmol, 1.0 equiv) in THF (75 mL) under nitrogen atmosphere was added 1M BH₃-THF (76 mL, 76.4 mmol, 3.0 equiv) dropwise at 0° C. The resulting mixture was stirred for 1 h at 25° C. The desired product could be detected by LCMS. The reaction was quenched with water (200 ml) at 0° C. The resulting mixture was extracted with EA (3×200 mL). The combined organic layers were washed with brine (1×600 mL), and dried over anhydrous Na₂SO₄. After filtration, the filtrate was concentrated under reduced pressure. The residue was purified by silica gel column chromatography, eluted with PE/EA (0% to 30% gradient in 20 min) to afford [4-(isopropylsulfanyl)phenyl]methanol.

ESI-MS m/z=165.20 [M-17]⁺; Calculated: 182.1.

¹H NMR (400 MHZ, DMSO-d₆) δ 7.37-7.25 (m, 4H), 5.19 (t, J=5.8 Hz, 1H), 4.48 (d, J=5.8 Hz, 2H), 3.47-3.36 (m, 1H), 1.21 (d, J=6.6 Hz, 6H).

### Step 3: [4-(Hydroxymethyl)phenyl](imino)isopropyl-lambda6-sulfanone

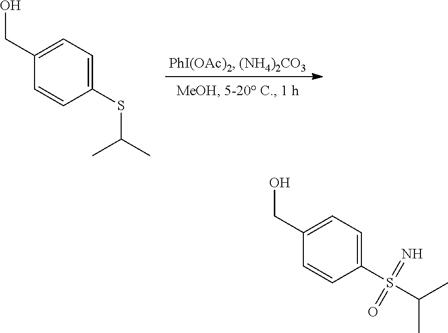

To a mixture of [4-(isopropylsulfanyl)phenyl]methanol (2.00 g, 10.9 mmol, 1.0 equiv) and (diacetoxyiodo)benzene (12.37 g, 38.4 mmol, 3.5 equiv) in MeOH (40 mL) was added (NH₄)₂CO₃ (2.42 g, 25.2 mmol, 2.3 equiv) in portions at 10° C. The resulting mixture was stirred for 1 h at 25° C. The desired product could be detected by LCMS. The resulting mixture was concentrated under reduced pressure. The residue was purified by silica gel column chromatography, eluted with DCM/MeOH (0% to 10% gradient in 20 min) to afford [4-(hydroxymethyl)phenyl](imino)isopropyl-lambda6-sulfanone.

ESI-MS m/z=214.20 [M+H]⁺; Calculated: 213.1.

¹H NMR (400 MHZ, DMSO-d₆) δ 7.86-7.75 (m, 2H), 7.53 (d, J=8.0 Hz, 2H), 5.42 (t, J=5.8 Hz, 1H), 4.60 (d, J=5.8 Hz, 2H), 4.06 (s, 1H), 3.24-3.16 (m, 1H), 1.03-1.08 (m, 6H).

### Step 4: [4-(Chloromethyl)phenyl](imino)isopropyl-lambda6-sulfanone

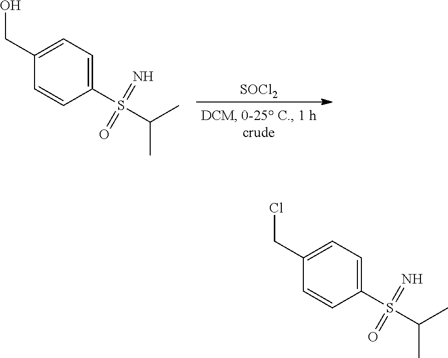

To a stirred solution of [4-(hydroxymethyl)phenyl](imino)isopropyl-lambda6-sulfanone (400 mg, 1.87 mmol, 1.0 equiv) in DCM (8 mL) was added SOCl₂ (446 mg, 3.75 mmol, 2.0 equiv) dropwise at 0° C. The resulting mixture was stirred for an additional 1 h at 25° C. The desired product could be detected by LCMS. The resulting mixture was concentrated under reduced pressure to afford [4-(chloromethyl)phenyl](imino)isopropyl-lambda6-sulfanone (crude).

ESI-MS m/z=232.00 [M+H]⁺; Calculated: 231.0.

### Step 5: 2-Cyclopropyl-6-isopropyl-4-(4-(propan-2-ylsulfonimidoyl)benzyl)-[1,2,4]triazolo[1,5-a]pyrimidin-7(4H)-one

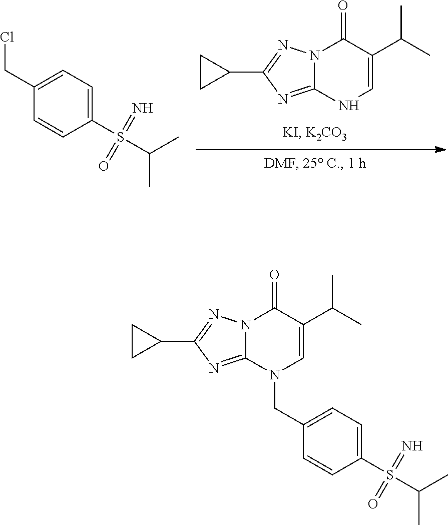 [SMILES: CC(C)S(=N)(=O)c1ccc(CCl)cc1 ; CC(C)c1c[nH]c2nc(C3CC3)nn2c1=O ; CC(C)c1cn(Cc2ccc(S(=N)(=O)C(C)C)cc2)c2nc(C3CC3)nn2c1=O]

A mixture of [4-(chloromethyl)phenyl](imino)isopropyl-lambda6-sulfanone (400 mg, 1.72 mmol, 1.0 equiv), 2-cyclopropyl-6-isopropyl-4H-[1,2,4]triazolo[1,5-a]pyrimidin-7-one (376 mg, 1.72 mmol, 1.0 equiv), KI (286 mg, 1.72 mmol, 1.0 equiv) and K₂CO₃ (1.20 g, 8.63 mmol, 5.0 equiv) in DMF (8 mL) was stirred for 1 h at 25° C. The desired product could be detected by LCMS. The resulting mixture was diluted with water (50 mL). The resulting mixture was extracted with DCM/IPA (5:1) (3×50 mL). The combined organic layers were washed with brine (3×100 mL), and dried over anhydrous Na₂SO₄. After filtration, the filtrate was concentrated under reduced pressure. The crude product (500 mg) was purified by Prep-HPLC with the following conditions (Column: YMC-Actus Triart C18 ExRS 30*150 mm, 5 μm; Mobile Phase A: water (10 mmol/L NH₄HCO₃), Mobile Phase B: ACN; Flow rate: 60 mL/min; Gradient: 21% B to 39% B in 10 min; Wave Length: 254 nm/220 nm; RT1 (min): 9.22) to afford 2-cyclopropyl-6-isopropyl-4-(4-(propan-2-ylsulfonimidoyl)benzyl)-[1,2,4]triazolo[1,5-a]pyrimidin-7(4H)-one.

ESI-MS m/z=414.20 [M+H]⁺; Calculated: 413.2.

¹H NMR (400 MHZ, DMSO-d₆) δ 8.02 (s, 1H), 7.90-7.77 (m, 2H), 7.64-7.48 (m, 2H), 5.55-5.32 (m, 2H), 4.19-4.05 (m, 1H), 3.26-3.14 (m, 1H), 3.05-2.92 (m, 1H), 2.11-1.96 (m, 1H), 1.25-1.15 (m, 6H), 1.14-1.05 (m, 6H), 1.03-0.94 (m, 2H), 0.92-0.82 (m, 2H).

### Compound 65: Rel-(R)-2-cyclopropyl-6-isopropyl-4-(3-methyl-4-(S-methylsulfonimidoyl)benzyl)-[1,2,4]triazolo[1,5-a]pyrimidin-7(4H)-one

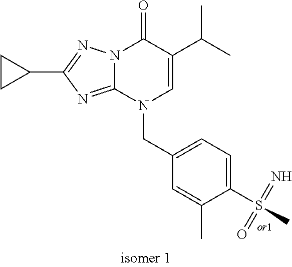 [SMILES: Cc1cc(Cn2cc(C(C)C)c(=O)n3nc(C4CC4)nc23)ccc1[S@](C)(=N)=O]

### Step 1: 2-Cyclopropyl-6-isopropyl-4-(3-methyl-4-(S-methylsulfonimidoyl)benzyl)-[1,2,4]triazolo[1,5-a]pyrimidin-7(4H)-one

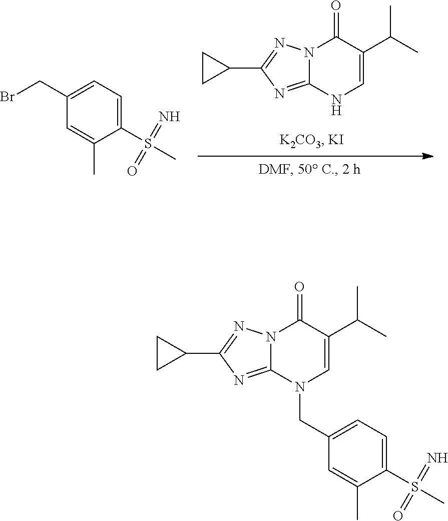 [SMILES: Cc1cc(CBr)ccc1S(C)(=N)=O ; CC(C)c1c[nH]c2nc(C3CC3)nn2c1=O ; Cc1cc(Cn2cc(C(C)C)c(=O)n3nc(C4CC4)nc23)ccc1S(C)(=N)=O]

A solution of [4-(bromomethyl)-2-methylphenyl](imino)methyl-lambda6-sulfanone (1.10 g, 4.19 mmol, 1.3 equiv), 2-cyclopropyl-6-isopropyl-4H-[1,2,4]triazolo[1,5-a]pyrimidin-7-one (710 mg, 3.25 mmol, 1.0 equiv), K₂CO₃ (1.35 g, 9.75 mmol, 3.0 equiv) and KI (540 mg, 3.25 mmol, 1.0 equiv) in DMF (10 mL) was stirred for 2 h at 50° C. under nitrogen atmosphere. The desired product could be detected by LCMS. The mixture was allowed to cool down to 25° C. The resulting mixture was diluted with EtOAc (100 mL). The resulting mixture was washed with 5×100 mL of water. The resulting mixture was concentrated under reduced pressure. The crude product (1.12 g) was purified by HP-flash with the following conditions (Column: YMC-Triart Prep C18-S column 100*250 mm, S-100 μm; Mobile Phase A: Water (10 mmol/L NH₄HCO₃), Mobile Phase B: MEOH; Flow rate: 140 mL/min mL/min; Gradient: 25% B to 55% B in 30 min; Wave Length: 254 nm/220 nm nm; RT1 (min): 20)) to afford 2-cyclopropyl-6-isopropyl-4-(3-methyl-4-(S-methylsulfonimidoyl)benzyl)-[1,2,4]triazolo[1,5-a]pyrimidin-7(4H)-one.

ESI-MS m/z=400.1 [M+H]⁺; Calculated: 399.2.

¹H NMR (400 MHZ, DMSO-d₆) δ 8.01 (s, 1H), 7.94 (d, J=8.1 Hz, 1H), 7.40 (s, 1H), 7.35 (d, J=8.2 Hz, 1H), 5.36 (s, 2H), 4.33 (s, 1H), 3.06 (s, 3H), 2.99 (p, J=6.9 Hz, 1H), 2.65 (s, 3H), 2.04 (tt, J=8.6, 4.9 Hz, 1H), 1.19 (d, J=6.9 Hz, 6H), 1.01 (dq, J=6.7, 3.8 Hz, 2H), 0.90 (p, J=4.0 Hz, 2H).

### Step 2: Rel-(R)-2-cyclopropyl-6-isopropyl-4-(3-methyl-4-(S-methylsulfonimidoyl)benzyl)-[1,2,4]triazolo[1,5-a]pyrimidin-7(4H)-one

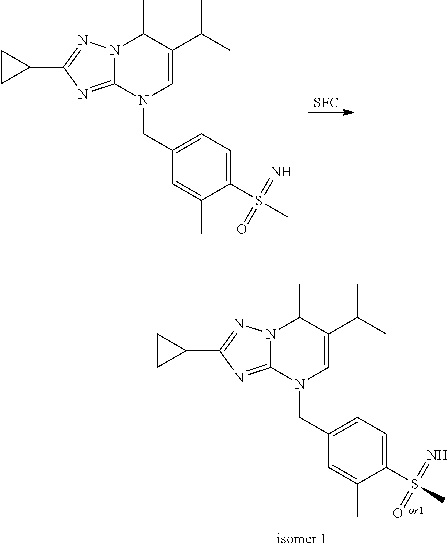 [SMILES: Cc1cc(CN2C=C(C(C)C)C(C)n3nc(C4CC4)nc32)ccc1S(C)(=N)=O ; "SFC" ; Cc1cc(CN2C=C(C(C)C)C(C)n3nc(C4CC4)nc32)ccc1[S@](C)(=N)=O]

The racemate (850 mg) was separated by SFC with the following conditions (Column: CHIRALPAK IH 3*25 cm, 5 μm; Mobile Phase A: CO₂, Mobile Phase B: MEOH; Flow rate: 100 mL/min; Gradient: isocratic 35% B; Column Temperature (C): 35; Back Pressure (bar): 100; Wave Length: 220/282 nm; RT1 (min): 6.32; Sample Solvent: MEOH; Injection Volume: 0.6 mL; Number Of Runs: 21), the pure fraction was concentrated and then lyophilized to afford rel-(R)-2-cyclopropyl-6-isopropyl-4-(3-methyl-4-(S-methylsulfonimidoyl)benzyl)-[1,2,4]triazolo[1,5-a]pyrimidin-7(4H)-one.

ESI-MS m/z=400.15 [M+H]⁺; Calculated: 399.2.

¹H NMR (400 MHZ, DMSO-d₆) δ 8.01 (s, 1H), 7.93 (d, J=8.1 Hz, 1H), 7.39 (d, J=1.9 Hz, 1H), 7.35 (d, J=8.1 Hz, 1H), 5.35 (s, 2H), 4.26 (s, 1H), 3.05 (s, 3H), 2.99 (p, J=6.9 Hz, 1H), 2.65 (s, 3H), 2.04 (tt, J=8.3, 5.0 Hz, 1H), 1.19 (d, J=6.9 Hz, 6H), 1.05-0.97 (m, 2H), 0.90 (dt, J=4.7, 3.0 Hz, 2H).

### Compound 66: Rel-(S)-2-cyclopropyl-6-isopropyl-4-(2-methyl-4-(S-methylsulfonimidoyl)benzyl)pyrazolo[1,5-a]pyrimidin-7(4H)-one

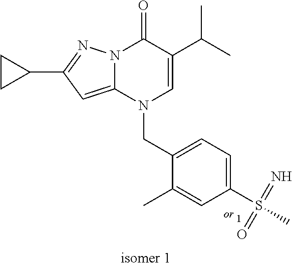 [SMILES: Cc1cc([S@@](C)(=N)=O)ccc1Cn1cc(C(C)C)c(=O)n2nc(C3CC3)cc12]

### Step 1: Rel-(S)-2-cyclopropyl-6-isopropyl-4-(2-methyl-4-(S-methylsulfonimidoyl)benzyl)pyrazolo[1,5-a]pyrimidin-7(4H)-one

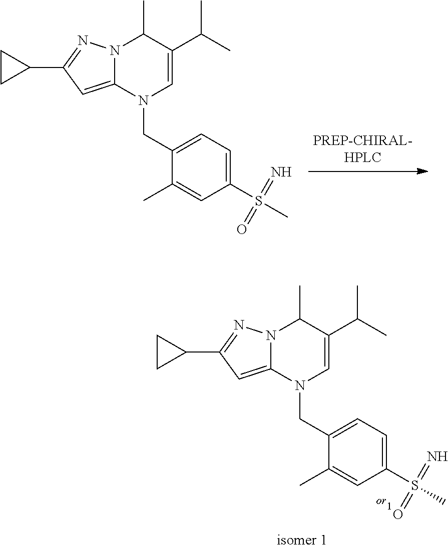 [SMILES: Cc1cc(S(C)(=N)=O)ccc1CN1C=C(C(C)C)C(C)n2nc(C3CC3)cc21 ; Cc1cc([S@@](C)(=N)=O)ccc1CN1C=C(C(C)C)C(C)n2nc(C3CC3)cc21]

The racemate (96 mg) was separated by PREP-CHIRAL-HPLC with the following conditions (Column: CHIRAL ART Cellulose-SZ, 3*25 cm, 5 μm; Mobile Phase A: Hex (10 mM NH₃-MeOH), Mobile Phase B: EtOH-HPLC; Flow rate: 40 mL/min; Gradient: isocratic 50; Wave Length: 200/212 nm; RT1 (min): 14.2; Sample Solvent: ETOH: DCM=1:1; Injection Volume: 1 mL; Number Of Runs: 5), the pure factions were concentrated under vacuum then lyophilized to afford rel-(S)-2-cyclopropyl-6-isopropyl-4-(2-methyl-4-(S-methylsulfonimidoyl)benzyl)pyrazolo[1,5-a]pyrimidin-7(4H)-one.

ESI-MS m/z=399.15 [M+H]⁺; Calculated: 398.2.

¹H NMR (400 MHZ, DMSO-d₆) δ 7.81 (s, 1H), 7.76 (s, 1H), 7.67 (d, J=8.0 Hz, 1H), 6.86 (d, J=8.1 Hz, 1H), 5.92 (s, 1H), 5.32 (s, 2H), 4.14 (s, 1H), 3.06-3.00 (m, 4H), 2.42 (s, 3H), 1.93 (td, J=8.4, 4.3 Hz, 1H), 1.17 (d, J=6.9 Hz, 6H), 0.96-0.88 (m, 2H), 0.75-0.69 (m, 2H).

### Compound 67: (S)-2-Cyclopropyl-6-isopropyl-4-(3-methyl-4-(S-methylsulfonimidoyl)benzyl)pyrazolo[1,5-a]pyrimidin-7(4H)-one

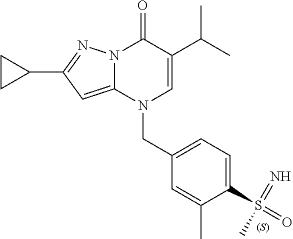

### Step 1: (S)-N-(tert-butyl(oxo)-l6-sulfaneylidene) pivalamide

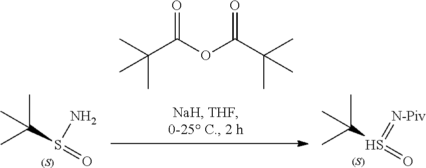

To a stirred solution of(S)-2-methylpropane-2-sulfinamide (20.00 g, 165.0 mmol, 1.0 equiv) in THF (200 mL) was added NaH (60% in mineral oil, 16.50 g, 412.5 mmol, 2.5 equiv) in portions at 0° C. under nitrogen atmosphere. The resulting mixture was stirred for 1 h at 0° C. under nitrogen atmosphere. To the above mixture was added trimethylacetic anhydride (33.81 g, 181.5 mmol, 1.1 equiv) dropwise over 15 min at 0° C. The resulting mixture was stirred for an additional 2 h at 25° C. The reaction was monitored by LCMS. The reaction was quenched with MeOH (20 mL) at 0° C., followed by the addition of brine (100 mL) and saturated aqueous NH₄Cl (100 mL). The aqueous layer was extracted with EtOAc (4×150 mL), combined organic layers were washed with saturated aqueous NaHCO₃ (100 mL) and brine (100 mL), dried over Na₂SO₄, filtered and concentrated. The residue was purified by silica gel column chromatography, eluted with PE/EA (3:1-2:1) to afford(S)-N-(tert-butyl(oxo)-l6-sulfaneylidene) pivalamide.

ESI-MS m/z=206.55 [M+H]⁺; Calculated: 205.1.

¹H NMR (400 MHZ, DMSO-d₆) δ 3.27 (s, 3H), 1.38 (s, 9H), 1.08 (s, 9H).

### Step 2: (S)-N-(tert-butyl(methyl)(oxo)-l6-sulfaneylidene) pivalamide

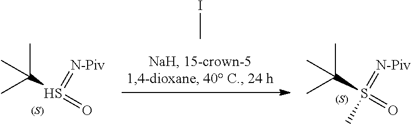

To a stirred solution of(S)-N-(tert-butyl(oxo)-l6-sulfaneylidene) pivalamide (20.00 g, 97.4 mmol, 1.0 equiv) in dehydrated dioxane (400 mL) was added NaH (60% in mineral oil, 4.68 g, 116.8 mmol, 1.2 equiv) and 15-crown-5 (25.75 g, 116.8 mmol, 1.2 equiv) at 25° C. After stirring for 10 min, methyl iodide (27.65 g, 194.8 mmol, 2.0 equiv) was added and the reaction mixture was heated to 40° C. for 24 h. The reaction was monitored by LCMS. The reaction mixture was cooled to 25° C. and quenched with NH₄Cl aq. (400 mL). The mixture was extracted with EtOAc (3×400 mL). The combined organic layers were washed with brine, dried over Na₂SO₄ and then concentrated. The residue was purified by silica gel column chromatography, eluted with PE/EA (4:1-3:1) to afford(S)-N-(tert-butyl(methyl)(oxo)-l6-sulfaneylidene) pivalamide.

ESI-MS m/z=220.10 [M+H]⁺; Calculated: 219.1.

¹H NMR (400 MHZ, DMSO-d₆) δ 3.27 (s, 3H), 1.38 (s, 9H), 1.08 (s, 9H)

### Step 3: (R)-N-(methyl(oxo)-l6-sulfaneylidene) pivalamide

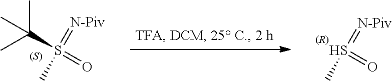

To a stirred solution of(S)-N-(tert-butyl(methyl)(oxo)-l6-sulfaneylidene) pivalamide (19.00 g, 86.6 mmol, 1.0 equiv) in DCM (200 mL) was added TFA (29.63 g, 259.8 mmol, 3.0 equiv) at 25° C. under nitrogen atmosphere. The resulting mixture was stirred for 2 h at 25° C. under nitrogen atmosphere. The reaction was monitored by LCMS. The reaction was quenched by the addition of NaHCO₃ aq. (100 mL) at 0° C. The resulting mixture was extracted with EtOAc (3×100 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EA (3:1-2:1) to afford (R)-N-(methyl(oxo)-l6-sulfaneylidene) pivalamide.

ESI-MS m/z=164.30 [M+H]⁺; Calculated: 163.1.

¹H NMR (400 MHZ, DMSO-d₆) δ 10.28 (s, 1H), 2.74 (s, 3H), 1.14 (s, 9H).

### Step 4: (S)-N-((4-(hydroxymethyl)-2-methylphenyl)(methyl)(oxo)-l6-sulfaneylidene) pivalamide

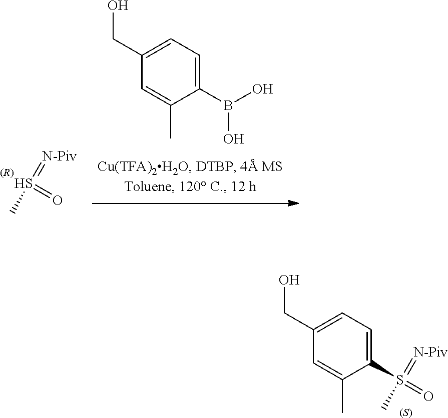

To a stirred solution of (R)-N-(methyl(oxo)-l6-sulfaneylidene) pivalamide (2.00 g, 12.2 mmol, 1.0 equiv) and [(2,2,2-trifluoroacetyl)oxy]cuprio 2,2,2-trifluoroacetate hydrate (753 mg, 2.45 mmol, 0.20 equiv) in toluene (30 mL) was added 4-(hydroxymethyl)-2-methylphenylboronic acid (4.07 g, 24.5 mmol, 2.0 equiv), 2-(tert-butylperoxy)-2-methylpropane (5.38 g, 36.6 mmol, 3.0 equiv) and 4 Å MS (2.00 g) at 25° C. under nitrogen atmosphere. The resulting mixture was stirred for 12 h at 120° C. under nitrogen atmosphere. The reaction was monitored by LCMS. The mixture was allowed to cool down to 25° C. The resulting mixture was concentrated under reduced pressure. The residue was purified by silica gel column chromatography, eluted with PE/EA (2:1-1:1) to afford(S)-N-((4-(hydroxymethyl)-2-methylphenyl)(methyl)(oxo)-l6-sulfaneylidene) pivalamide.

ESI-MS m/z=284.1 [M+H]⁺; Calculated: 283.1.

¹H NMR (400 MHZ, DMSO-d₆) δ 7.88 (d, J=8.2 Hz, 1H), 7.37 (s, 2H), 5.41 (t, J=5.7 Hz, 1H), 4.56 (d, J=5.7 Hz, 2H), 3.33 (d, J=1.3 Hz, 3H), 2.59 (s, 3H), 1.10 (s, 9H).

### Step 5: (S)-(4-(hydroxymethyl)-2-methylphenyl)(imino)(methyl)-l6-sulfanone

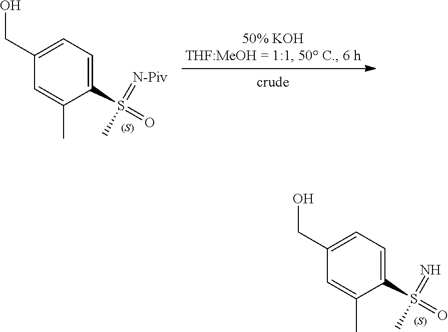

To a stirred solution of(S)-N-((4-(hydroxymethyl)-2-methylphenyl)(methyl)(oxo)-l6-sulfaneylidene) pivalamide (400 mg, 1.41 mmol, 1.0 equiv) in MeOH (5 mL) and THF (5 mL) was added 50% KOH aq. (4 mL) at 25° C. under nitrogen atmosphere. The resulting mixture was stirred for 6 h at 50° C. under nitrogen atmosphere. The reaction was monitored by LCMS. The resulting mixture was diluted with water (20 mL). The resulting mixture was extracted with CHCl3/IPA=3/1 (3×40 mL), and dried over anhydrous Na₂SO₄. After filtration, the filtrate was concentrated under reduced pressure. This resulted in(S)-[4-(hydroxymethyl)-2-methylphenyl](imino)methyl-lambda6-sulfanone (crude).

ESI-MS m/z=200.1 [M+H]⁺; Calculated: 199.1.

### Step 6: (S)-(4-(bromomethyl)-2-methylphenyl)(imino)(methyl)-l6-sulfanone

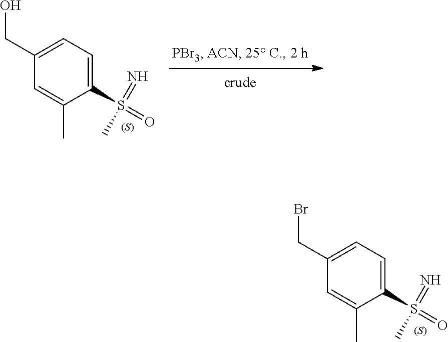

To a stirred solution of(S)-[4-(hydroxymethyl)-2-methylphenyl](imino)methyl-lambda6-sulfanone (350 mg, 1.75 mmol, 1.0 equiv) in ACN (10 mL) was added PBr₃ (475 mg, 1.75 mmol, 1.0 equiv) at 25° C. under nitrogen atmosphere. The resulting mixture was stirred for 2 h at 25° C. under nitrogen atmosphere. The reaction was monitored by LCMS. The resulting mixture was concentrated under reduced pressure. The residue was purified by trituration with ethoxyethane (5 mL). This resulted in(S)-(4-(bromomethyl)-2-methylphenyl)(imino)(methyl)-l6-sulfanone (crude).

ESI-MS m/z=262.0 [M+H]⁺; Calculated: 261.0.

### Step 7: (S)-2-cyclopropyl-6-isopropyl-4-(3-methyl-4-(S-methylsulfonimidoyl)benzyl)pyrazolo[1,5-a]pyrimidin-7(4H)-one

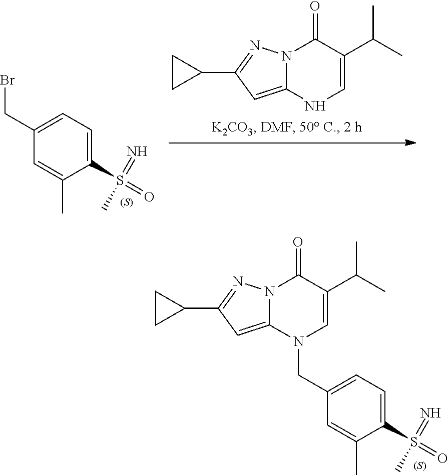

To a solution of(S)-(4-(bromomethyl)-2-methylphenyl)(imino)(methyl)-l6-sulfanone (560 mg, 2.13 mmol, 1.0 equiv) and 2-cyclopropyl-6-isopropyl-4H-pyrazolo[1,5-a]pyrimidin-7-one (556 mg, 2.56 mmol, 1.2 equiv) in DMF (6 mL) was added K₂CO₃ (885 mg, 6.40 mmol, 3.0 equiv) at 25° C. under nitrogen atmosphere. The resulting mixture was stirred for 2 h at 50° C. under nitrogen atmosphere. The reaction was monitored by LCMS. The resulting mixture was diluted with water (20 mL). The resulting mixture was extracted with CHCl3/IPA=3/1 (3×30 mL), and dried over anhydrous Na₂SO₄. After filtration, the filtrate was concentrated under reduced pressure. The crude product (400 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min mL/min; Gradient: 12% B to 39% B in 7 min; Wave Length: 254 nm/220 nm nm; RT1 (min): 6.6) to afford(S)-2-cyclopropyl-6-isopropyl-4-(3-methyl-4-(S-methylsulfonimidoyl)benzyl)pyrazolo[1,5-a]pyrimidin-7(4H)-one.

The enantiomeric excess can be improved via the following recrystallization procedure:

The racemate (200 mg) was dissolved in 4 mL EtOAc, and then added 0.56 mL ACN heated at 80° C. for 10 min until the solid completely dissolved. The solution is slow cooled at 25° C. overnight and 4° C. for 1 day. The mother liquor was collected and concentrated under reduced pressure to afford(S)-2-cyclopropyl-6-isopropyl-4-(3-methyl-4-(S-methylsulfonimidoyl)benzyl)pyrazolo[1,5-a]pyrimidin-7(4H)-one.

ESI-MS m/z=399.20 [M+H]⁺; Calculated: 398.2.

¹H NMR (400 MHZ, DMSO-d₆) δ 7.94 (d, J=8.1 Hz, 2H), 7.38-7.27 (m, 2H), 6.03 (s, 1H), 5.26 (s, 2H), 4.27 (s, 1H), 3.05 (d, J=1.2 Hz, 3H), 3.01 (q, J=6.9 Hz, 1H), 2.66 (s, 3H), 1.99-1.88 (m, 1H), 1.20 (d, J=6.9 Hz, 6H), 0.95 (s, 2H), 0.79-0.72 (m, 2H).

### Compound 68: Rel-(S)-2-cyclopropyl-6-isopropyl-4-(4-(propan-2-ylsulfonimidoyl)benzyl)-[1,2,4]triazolo[1,5-a]pyrimidin-7(4H)-one

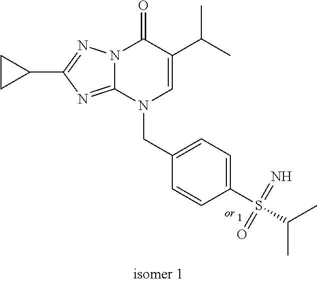 [SMILES: CC(C)c1cn(Cc2ccc([S@@](=N)(=O)C(C)C)cc2)c2nc(C3CC3)nn2c1=O]

### Step 1: Rel-(S)-2-cyclopropyl-6-isopropyl-4-(4-(propan-2-ylsulfonimidoyl)benzyl)-[1,2,4]triazolo[1,5-a]pyrimidin-7(4H)-one

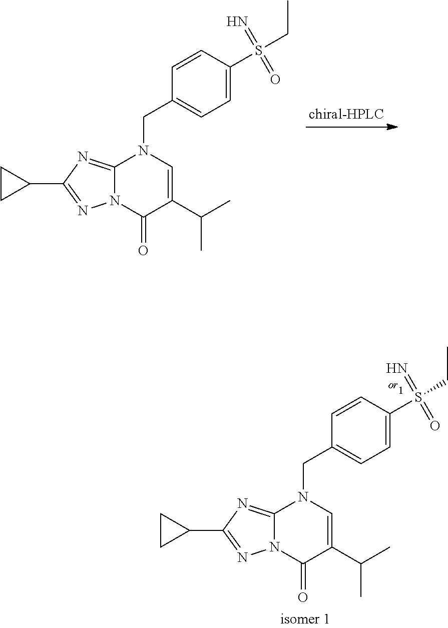 [SMILES: CCS(=N)(=O)c1ccc(Cn2cc(C(C)C)c(=O)n3nc(C4CC4)nc23)cc1 ; CC[S@](=N)(=O)c1ccc(Cn2cc(C(C)C)c(=O)n3nc(C4CC4)nc23)cc1]

The racemate (200 mg) was separated by Prep-CHIRAL-HPLC with the following conditions (Column: CHIRAL ART Cellulose-SZ, 3*25 cm, 5 μm; Mobile Phase A: Hex (10 mM NH₃-MeOH), Mobile Phase B: ETOH; Flow rate: 40 mL/min; Gradient: isocratic 50; Wave Length: 210/284 nm; RT1 (min): 12.7:16; Sample Solvent: ETOH), the pure factions were concentrated under vacuum then lyophilized to afford rel-(S)-2-cyclopropyl-6-isopropyl-4-(4-(propan-2-ylsulfonimidoyl)benzyl)-[1,2,4]triazolo[1,5-a]pyrimidin-7(4H)-one.

ESI-MS m/z=414.05 [M+H]⁺; Calculated: 413.2.

¹H NMR (400 MHZ, DMSO-d₆) δ 8.03 (s, 1H), 7.89-7.79 (m, 2H), 7.56 (d, J=8.2 Hz, 2H), 5.43 (s, 2H), 4.14 (s, 1H), 3.27-2.92 (m, 2H), 2.09-1.98 (m, 1H), 1.19 (d, J=6.9 Hz, 6H), 1.14-1.06 (m, 6H), 1.03-0.96 (m, 2H), 0.92-0.83 (m, 2H).

### Compound 69: Rel-(R)-2-cyclopropyl-4-(2-fluoro-4-(S-methylsulfonimidoyl)benzyl)-6-isopropyl-[1,2,4]triazolo[1,5-a]pyrimidin-7(4H)-one

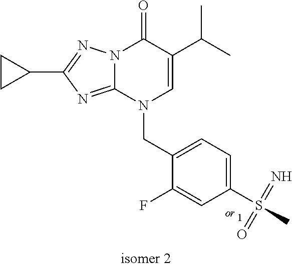 [SMILES: CC(C)c1cn(Cc2ccc([S@](C)(=N)=O)cc2F)c2nc(C3CC3)nn2c1=O]

### Step 1: Rel-(R)-2-cyclopropyl-4-(2-fluoro-4-(S-methylsulfonimidoyl)benzyl)-6-isopropyl-[1,2,4]triazolo[1,5-a]pyrimidin-7(4H)-one

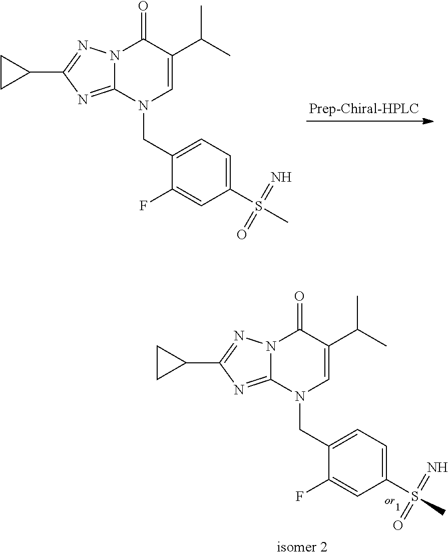 [SMILES: CC(C)c1cn(Cc2ccc(S(C)(=N)=O)cc2F)c2nc(C3CC3)nn2c1=O ; CC(C)c1cn(Cc2ccc([S@](C)(=N)=O)cc2F)c2nc(C3CC3)nn2c1=O]

The racemate (90 mg) was separated by Prep-CHIRAL-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 209/290 nm; RT1 (min): 17.8; Sample Solvent: EtOH), the pure factions were concentrated under vacuum then lyophilized to afford rel-(R)-2-cyclopropyl-4-(2-fluoro-4-(S-methylsulfonimidoyl)benzyl)-6-isopropyl-[1,2,4]triazolo[1,5-a]pyrimidin-7(4H)-one.

ESI-MS m/z=403.95 [M+H]⁺; Calculated: 403.1.

¹H NMR (400 MHZ, DMSO-d₆) δ 7.99 (s, 1H), 7.74 (dd, J=19.2, 8.9 Hz, 2H), 7.50 (t, J=7.6 Hz, 1H), 5.46 (s, 2H), 4.39 (s, 1H), 3.09 (s, 3H), 3.04-2.94 (m, 1H), 2.08-1.96 (m, 1H), 1.19 (d, J=6.9 Hz, 6H), 1.03-0.94 (m, 2H), 0.90-0.79 (m, 2H).

¹⁹F NMR (377 MHZ, DMSO) δ −114.96.

### Compound 70: Rel-(S)-2-cyclopropyl-4-(2-fluoro-4-(S-methylsulfonimidoyl)benzyl)-6-isopropylpyrazolo[1,5-a]pyrimidin-7(4H)-one

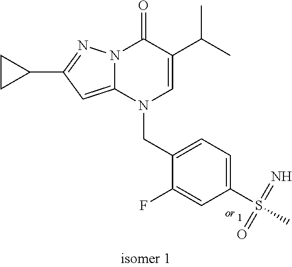 [SMILES: CC(C)c1cn(Cc2ccc([S@@](C)(=N)=O)cc2F)c2cc(C3CC3)nn2c1=O]

### Step 1: Rel-(S)-2-Cyclopropyl-4-(2-fluoro-4-(S-methylsulfonimidoyl)benzyl)-6-isopropylpyrazolo[1,5-a]pyrimidin-7(4H)-one

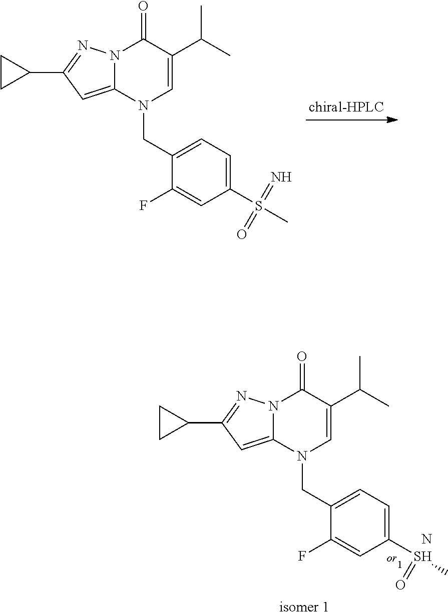

The racemate (80 mg) was separated by PREP-CHIRAL-HPLC with the following conditions (Column: CHIRALPAK-IK, 3*25 mm, 5 μm; Mobile Phase A: Hex (10 mM NH₃-MeOH), Mobile Phase B: EtOH; Flow rate: 208/242 mL/min; Gradient: isocratic 50; Wave Length: 40 nm; RT1 (min): 13.503; Sample Solvent: EtOH: MeOH=1:2-HPLC; Injection Volume: 0.5 mL; Number Of Runs: 2), the pure factions were concentrated under vacuum then lyophilized to afford rel-(S)-2-Cyclopropyl-4-(2-fluoro-4-(S-methylsulfonimidoyl)benzyl)-6-isopropylpyrazolo[1,5-a]pyrimidin-7(4H)-one.

ESI-MS m/z=403.15 [M+H]⁺; Calculated: 402.2.

¹H NMR (400 MHZ, DMSO-d₆) δ 7.86 (s, 1H), 7.79-7.70 (m, 2H), 7.47 (t, J=7.6 Hz, 1H), 6.01 (s, 1H), 5.38 (s, 2H), 4.36 (s, 1H), 3.09 (d, J=1.1 Hz, 3H), 3.01-2.97 (m, 1H), 1.94 (tt, J=8.3, 4.9 Hz, 1H), 1.18 (d, J=6.9 Hz, 6H), 0.98-0.89 (m, 2H), 0.78-0.70 (m, 2H).

¹⁹F NMR (376 MHZ, DMSO) δ −114.48.

### Compound 71: 2-Cyclopropyl-6-isopropyl-4-(3-methyl-4-(S-methylsulfonimidoyl)benzyl)-[1,2,4]triazolo[1,5-a]pyrimidin-7(4H)-one

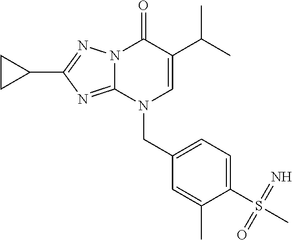 [SMILES: Cc1cc(Cn2cc(C(C)C)c(=O)n3nc(C4CC4)nc23)ccc1S(C)(=N)=O]

### Step 1: 2-Cyclopropyl-6-isopropyl-4-(3-methyl-4-(S-methylsulfonimidoyl)benzyl)-[1,2,4]triazolo[1,5-a]pyrimidin-7(4H)-one

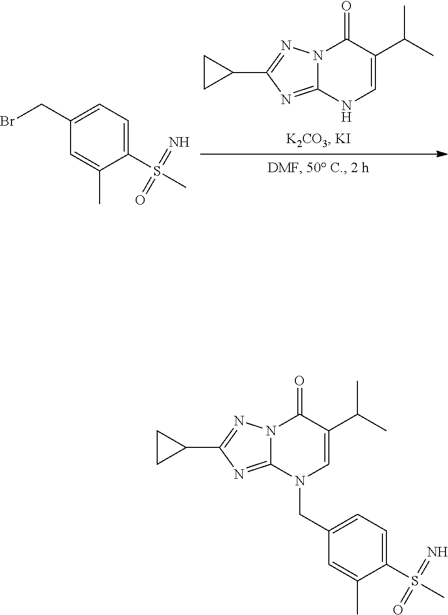 [SMILES: Cc1cc(CBr)ccc1S(C)(=N)=O ; CC(C)c1c[nH]c2nc(C3CC3)nn2c1=O ; Cc1cc(Cn2cc(C(C)C)c(=O)n3nc(C4CC4)nc23)ccc1S(C)(=N)=O]

A solution of [4-(bromomethyl)-2-methylphenyl](imino)methyl-lambda6-sulfanone (359 mg, 1.37 mmol, 1.3 equiv), 2-cyclopropyl-6-isopropyl-4H-[1,2,4]triazolo[1,5-a]pyrimidin-7-one (230 mg, 1.05 mmol, 1.0 equiv), K₂CO₃ (436 mg, 3.16 mmol, 3.0 equiv) and KI (174 mg, 1.05 mmol, 1.0 equiv) in DMF (5 mL) was stirred for 2 h at 50° C. under nitrogen atmosphere. The desired product could be detected by LCMS. The mixture was allowed to cool down to 25° C. The resulting mixture was diluted with EtOAc (50 mL). The resulting mixture was washed with 5×50 ml of water. The resulting mixture was concentrated under reduced pressure. The crude product (250 mg) was purified by Prep-HPLC with the following conditions (Column: XSelect CSH Fluoro Phenyl 30*150 mm, 5 m; Mobile Phase A: Water (0.1% FA), Mobile Phase B: ACN; Flow rate: 60 mL/min mL/min; Gradient: 5% B to 5% B in 1.5 min, 5% B to 17% B in 2 min, 17% B to 38% B in 10 min; Wave Length: 254 nm/220 nm nm; RT1 (min): 8.72), the pure fraction was concentrated then lyophilized to afford 2-cyclopropyl-6-isopropyl-4-(3-methyl-4-(S-methylsulfonimidoyl)benzyl)-[1,2,4]triazolo[1,5-a]pyrimidin-7(4H)-one.

ESI-MS m/z=440.10 [M+H]⁺; Calculated: 399.2.

¹H NMR (400 MHZ, DMSO-d₆) δ 8.01 (s, 1H), 7.94 (d, J=8.1 Hz, 1H), 7.40 (s, 1H), 7.35 (d, J=8.2 Hz, 1H), 5.36 (s, 2H), 4.33 (s, 1H), 3.06 (s, 3H), 2.99 (p, J=6.9 Hz, 1H), 2.65 (s, 3H), 2.04 (tt, J=8.6, 4.9 Hz, 1H), 1.19 (d, J=6.9 Hz, 6H), 1.04-0.98 (m, 2H), 0.94-0.88 (m, 2H).

### Compound 72: Rel-(R)-2-cyclopropyl-4-(3-fluoro-4-(S-methylsulfonimidoyl)benzyl)-6-isopropylpyrazolo[1,5-a]pyrimidin-7(4H)-one

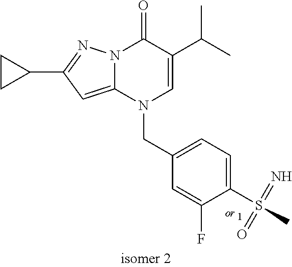 [SMILES: CC(C)c1cn(Cc2ccc([S@](C)(=N)=O)c(F)c2)c2cc(C3CC3)nn2c1=O]

### Step 1: Rel-(R)-2-cyclopropyl-4-(3-fluoro-4-(S-methylsulfonimidoyl)benzyl)-6-isopropylpyrazolo[1,5-a]pyrimidin-7(4H)-one

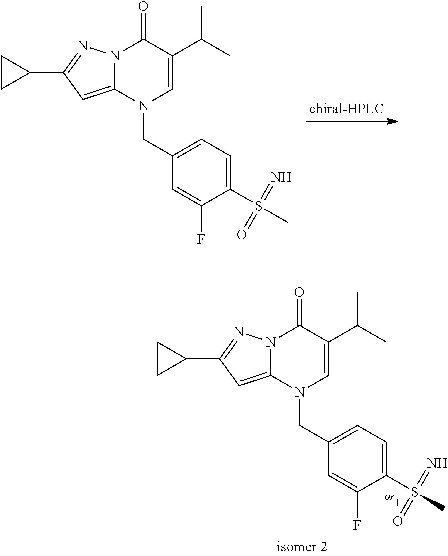 [SMILES: CC(C)c1cn(Cc2ccc(S(C)(=N)=O)c(F)c2)c2cc(C3CC3)nn2c1=O ; CC(C)c1cn(Cc2ccc([S@](C)(=N)=O)c(F)c2)c2cc(C3CC3)nn2c1=O]

The racemate (100 mg) was separated by Chiral-HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50; Wave Length: 220/254 nm; RT1 (min): 9.18; Sample Solvent: ETOH: DCM=1:1; Injection Volume: 0.7 mL; Number Of Runs: 9), the pure fraction was concentrated and then lyophilized to afford rel-(R)-2-cyclopropyl-4-(3-fluoro-4-(S-methylsulfonimidoyl)benzyl)-6-isopropylpyrazolo[1,5-a]pyrimidin-7(4H)-one.

ESI-MS m/z=403.25 [M+H]⁺; Calculated: 402.2.

¹H NMR (400 MHZ, DMSO-d₆) δ 7.93 (s, 1H), 7.89-7.80 (m, 1H), 7.49-7.41 (m, 1H), 7.36-7.29 (m, 1H), 6.04 (s, 1H), 5.29 (s, 2H), 4.70 (d, J=1.5 Hz, 1H), 3.16 (d, J=1.4 Hz, 3H), 3.06-2.93 (m, 1H), 1.99-1.88 (m, 1H), 1.20 (d, J=6.9 Hz, 3H), 1.20 (d, J=6.9 Hz, 3H), 0.98-0.88 (m, 2H), 0.79-0.70 (m, 2H).

¹⁹F NMR (376 MHZ, DMSO-d₆) δ 109.39.

### Compound 73 Rac: 2-Cyclopropyl-3-fluoro-4-(2-fluoro-4-(S-methylsulfonimidoyl)benzyl)-6-isopropylpyrazolo[1,5-a]pyrimidin-7(4H)-one

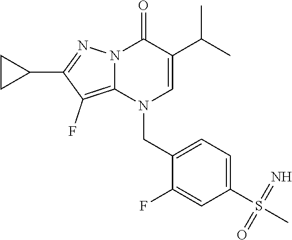 [SMILES: CC(C)c1cn(Cc2ccc(S(C)(=N)=O)cc2F)c2c(F)c(C3CC3)nn2c1=O]

### Step 1: 2-Cyclopropyl-3-fluoro-6-isopropylpyrazolo[1,5-a]pyrimidin-7(4H)-one

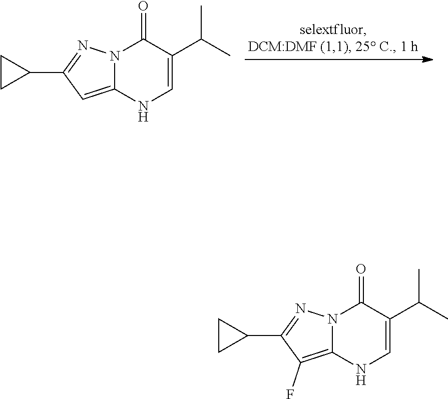 [SMILES: CC(C)c1c[nH]c2cc(C3CC3)nn2c1=O ; CC(C)c1c[nH]c2c(F)c(C3CC3)nn2c1=O]

To a stirred mixture of 2-cyclopropyl-6-isopropyl-4H-pyrazolo[1,5-a]pyrimidin-7-one (500 mg, 2.30 mmol, 1.0 equiv) in DCM (12.5 mL) and DMF (12.5 mL) was added Selectfluor (735 mg, 2.07 mmol, 0.90 equiv) at 25° C. under air atmosphere. The resulting mixture was stirred for 1 h at 25° C. under nitrogen atmosphere. The desired product could be detected by LCMS. The resulting mixture was concentrated under reduced pressure. The residue was purified by silica gel column chromatography, eluted with CH₂Cl₂/MeOH (10:1) to afford 2-cyclopropyl-3-fluoro-6-isopropylpyrazolo[1,5-a]pyrimidin-7(4H)-one.

ESI-MS m/z=236.11 [M+H]⁺; Calculated: 235.1.

¹H NMR (400 MHZ, DMSO-d₆) δ 12.39 (s, 1H), 7.59 (s, 1H), 2.03-1.97 (m, 1H), 1.68-1.61 (m, 1H), 1.25-1.22 (m, 2H), 1.16 (d, J=6.9 Hz, 6H), 0.92-0.92 (m, 2H).

¹⁹F NMR (377 MHZ, DMSO) δ −189.94.

### Step 2: 2-Cyclopropyl-3-fluoro-4-(2-fluoro-4-(S-methylsulfonimidoyl)benzyl)-6-isopropylpyrazolo[1,5-a]pyrimidin-7(4H)-one

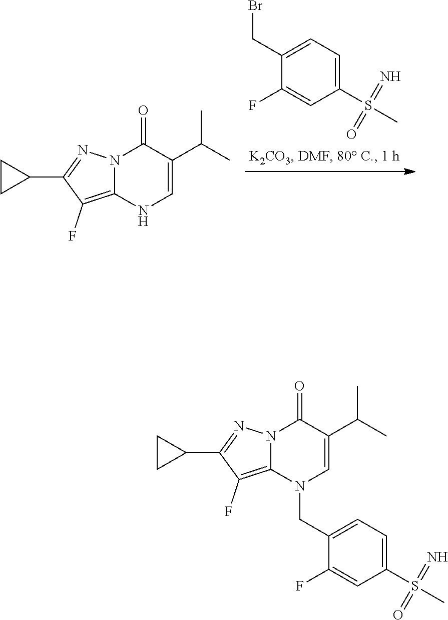 [SMILES: CC(C)c1c[nH]c2c(F)c(C3CC3)nn2c1=O ; CS(=N)(=O)c1ccc(CBr)c(F)c1 ; CC(C)c1cn(Cc2ccc(S(C)(=N)=O)cc2F)c2c(F)c(C3CC3)nn2c1=O]

To a stirred mixture of 2-cyclopropyl-3-fluoro-6-isopropylpyrazolo[1,5-a]pyrimidin-7(4H)-one (40 mg, 0.17 mmol, 1.0 equiv) in DMF (1.0 mL) was added K₂CO₃ (70 mg, 0.51 mmol, 3.0 equiv) and [4-(bromomethyl)-3-fluorophenyl](imino)methyl-lambda6-sulfanone (50 mg, 0.19 mmol, 1.1 equiv) at 25° C. under air atmosphere. The resulting mixture was stirred for 1 h at 80° C. under nitrogen atmosphere. The desired product could be detected by LCMS. The resulting mixture was diluted with water (10 mL). The aqueous layer was extracted with CHCl3/IPA (3×10 mL). The combined organic layers were dried over anhydrous Na₂SO₄. After filtration, the filtrate was concentrated under reduced pressure. The crude product (40 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min; Gradient: 32% B to 60% B in 7 min; Wave Length: 254 nm/220 nm; RT1 (min): 6.65), the pure fraction was concentrated under reduced pressure and then lyophilized to afford 2-cyclopropyl-3-fluoro-4-(2-fluoro-4-(S-methylsulfonimidoyl)benzyl)-6-isopropylpyrazolo[1,5-a]pyrimidin-7(4H)-one.

ESI-MS m/z=421.10 [M+H]⁺; Calculated: 420.1.

¹H NMR (400 MHZ, DMSO-d₆) δ 7.89 (s, 1H), 7.80-7.72 (m, 2H), 7.40-7.36 (m, 1H), 5.49 (s, 2H), 4.39 (s, 1H), 3.10 (s, 3H), 3.03-2.98 (m, 1H), 1.98-1.93 (m, 1H), 1.18 (d, J=6.9 Hz, 6H), 1.00-0.96 (m, 2H), 0.90-0.87 (m, 2H).

¹⁹F NMR (377 MHz, DMSO) δ −115.54, −115.55

### Compound 73 A: Rel-(S)-2-cyclopropyl-3-fluoro-4-(2-fluoro-4-(S-methylsulfonimidoyl)benzyl)-6-isopropylpyrazolo[1,5-a]pyrimidin-7(4H)-one

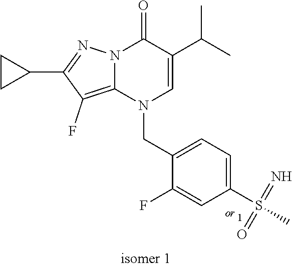 [SMILES: CC(C)c1cn(Cc2ccc([S@@](C)(=N)=O)cc2F)c2c(F)c(C3CC3)nn2c1=O]

### Step 1: 2-Cyclopropyl-6-isopropyl-4-((2-(trimethylsilyl) ethoxy)methyl) pyrazolo[1,5-a]pyrimidin-7(4H)-one

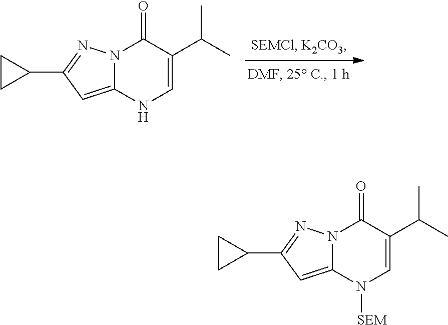 [SMILES: CC(C)c1c[nH]c2cc(C3CC3)nn2c1=O ; CC(C)c1cn(COCC[Si](C)(C)C)c2cc(C3CC3)nn2c1=O]

To a stirred mixture of 2-cyclopropyl-6-isopropyl-4H-pyrazolo[1,5-a]pyrimidin-7-one (2.00 g, 9.21 mmol, 1.0 equiv) in DMF (60.00 mL) was added K₂CO₃ (3.82 g, 27.6 mmol, 3.0 equiv) and [2-(chloromethoxy)ethyl]trimethylsilane (1.53 g, 9.21 mmol, 1.0 equiv) at 25° C. under air atmosphere. The resulting mixture was stirred for 1 h at 25° C. under nitrogen atmosphere. The desired product could be detected by LCMS. The resulting mixture was diluted with water (200 mL). The resulting mixture was extracted with EtOAc (2×200 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (3:1-1:1) to afford 2-Cyclopropyl-6-isopropyl-4-((2-(trimethylsilyl) ethoxy)methyl) pyrazolo[1,5-a]pyrimidin-7(4H)-one.

ESI-MS m/z=348.0 [M+H]⁺; Calculated: 347.0.

### Step 2: 2-Cyclopropyl-3-fluoro-6-isopropyl-4-((2-(trimethylsilyl) ethoxy)methyl) pyrazolo[1,5-a]pyrimidin-7(4H)-one

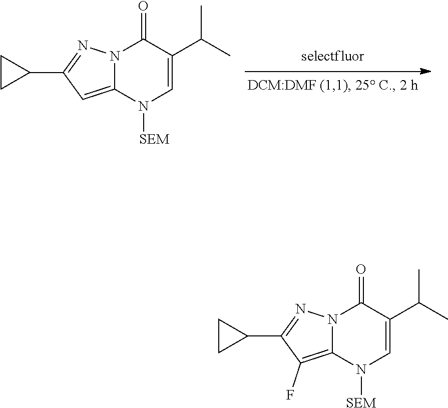 [SMILES: CC(C)c1cn(COCC[Si](C)(C)C)c2cc(C3CC3)nn2c1=O ; CC(C)c1cn(COCC[Si](C)(C)C)c2c(F)c(C3CC3)nn2c1=O]

To a solution of 2-cyclopropyl-6-isopropyl-4{[2-(trimethylsilyl) ethoxy]methyl}pyrazolo[1,5-a]pyrimidin-7-one (700 mg, 2.01 mmol, 1.0 equiv) and Selectfluor (642 mg, 1.81 mmol, 0.90 equiv) in DMF (5.00 mL) at 25° C. under nitrogen atmosphere. The resulting mixture was stirred for 2 h at 25° C. under nitrogen atmosphere. The reaction was monitored by LCMS. The residue was purified by reversed-phase flash chromatography with the following conditions: column, C18; mobile phase, MeCN in Water (0.1% FA), 10% to 50% gradient in 40 min; detector, UV 254 nm to afford 2-Cyclopropyl-3-fluoro-6-isopropyl-4-((2-(trimethylsilyl) ethoxy)methyl) pyrazolo[1,5-a]pyrimidin-7(4H)-one.

ESI-MS m/z=366.1 [M+H]⁺; Calculated: 365.2.

¹H NMR (400 MHZ, DMSO-d₆) δ 7.99-7.74 (m, 1H), 5.41 (d, J=1.2 Hz, 2H), 3.58 (t, J=7.9 Hz, 2H), 2.98 (p, J=6.9 Hz, 1H), 2.17-1.93 (m, 1H), 1.17 (d, J=6.9 Hz, 6H), 1.07-0.99 (m, 2H), 0.96-0.90 (m, 2H), 0.89-0.82 (m, 2H), −0.08 (s, 9H).

¹⁹F NMR (376 MHZ, DMSO) δ −186.88.

### Step 3: 2-Cyclopropyl-3-fluoro-6-isopropylpyrazolo[1,5-a]pyrimidin-7(4H)-one

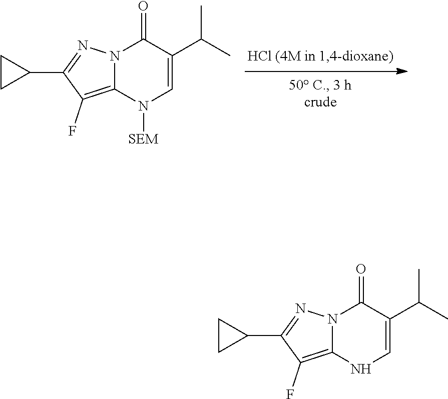 [SMILES: CC(C)c1cn(COCC[Si](C)(C)C)c2c(F)c(C3CC3)nn2c1=O ; CC(C)c1c[nH]c2c(F)c(C3CC3)nn2c1=O]

To a solution of 2-cyclopropyl-3-fluoro-6-isopropyl-4{[2-(trimethylsilyl) ethoxy]methyl}pyrazolo[1,5-a]pyrimidin-7-one (180 mg, 0.49 mmol, 1.0 equiv) in HCl (g, 4 M in 1,4-dioxane, 3.00 mL) at 25° C. under nitrogen atmosphere. The resulting mixture was stirred for 3 h at 50° C. under nitrogen atmosphere. The reaction was monitored by LCMS. The resulting mixture was concentrated under reduced pressure. The residue was purified by trituration with ethyl ether (5.00 mL), to afford 2-cyclopropyl-3-fluoro-6-isopropylpyrazolo[1,5-a]pyrimidin-7(4H)-one (crude).

ESI-MS m/z=236.1 [M+H]⁺; Calculated: 235.1.

### Step 4: 2-Cyclopropyl-3-fluoro-4-(2-fluoro-4-(S-methylsulfonimidoyl)benzyl)-6-isopropylpyrazolo[1,5-a]pyrimidin-7(4H)-one

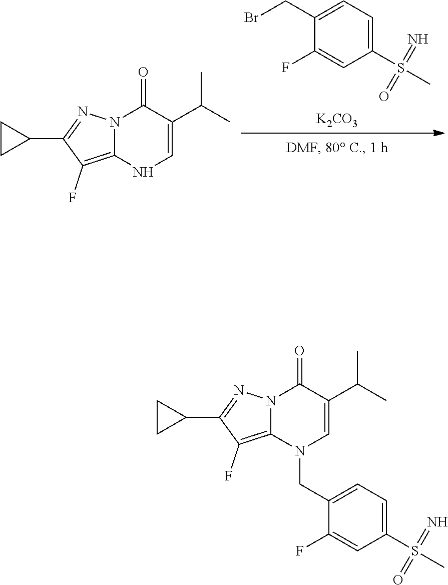

To a stirred mixture of 2-cyclopropyl-3-fluoro-6-isopropyl-4H-pyrazolo[1,5-a]pyrimidin-7-one (140 mg, 0.59 mmol, 1.0 equiv) in DMF (4.00 mL) was added K₂CO₃ (247 mg, 1.79 mmol, 3.0 equiv) and [4-(bromomethyl)-3-fluorophenyl](imino)methyl-lambda6-sulfanone (174 mg, 0.65 mmol, 1.1 equiv) at 25° C. under air atmosphere. The resulting mixture was stirred for 1 h at 80° C. under nitrogen atmosphere. The desired product could be detected by LCMS. The resulting mixture was diluted with water (10 mL). The aqueous layer was extracted with CHCl3/IPA (3:1, 3×10 mL). The combined organic layers were dried over anhydrous Na₂SO₄. After filtration, the filtrate was concentrated under reduced pressure. The crude product was purified by Prep-HPLC with the following conditions (Column: XBridge Prep OBD C18 Column 30*150 mm, 5 m; Mobile Phase A: 10 mmol NH₄HCO₃+0.05% NH₃H₂O, Mobile Phase B: ACN; Flow rate: 60 mL/min mL/min; Gradient: 26% B to 37% B in 10 min; Wave Length: 254 nm/220 nm nm; RT1 (min): 9.9) to afford 2-cyclopropyl-3-fluoro-4-(2-fluoro-4-(S-methylsulfonimidoyl)benzyl)-6-isopropylpyrazolo[1,5-a]pyrimidin-7(4H)-one.

ESI-MS m/z=421.15 [M+H]⁺; Calculated: 420.1.

¹H NMR (400 MHZ, DMSO-d₆) δ 7.89 (s, 1H), 7.82-7.72 (m, 2H), 7.38 (t, J=7.6 Hz, 1H), 5.50 (s, 2H), 4.40 (d, J=1.6 Hz, 1H), 3.10 (d, J=1.2 Hz, 3H), 3.06-2.91 (m, J=6.5 Hz, 1H), 1.95 (tt, J=8.3, 5.0 Hz, 1H), 1.19 (d, J=6.9 Hz, 6H), 1.03-0.93 (m, 2H), 0.93-0.83 (m, 2H).

¹⁹F NMR (377 MHz, DMSO) δ −115.53, −188.47, −188.48.

### Step 5: Rel-(S)-2-cyclopropyl-3-fluoro-4-(2-fluoro-4-(S-methylsulfonimidoyl)benzyl)-6-isopropylpyrazolo[1,5-a]pyrimidin-7(4H)-one

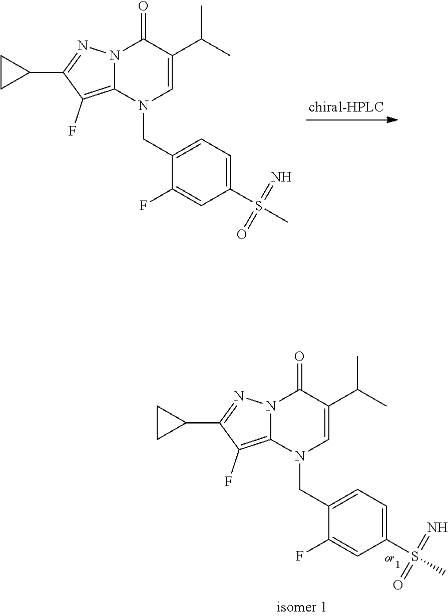 [SMILES: CC(C)c1cn(Cc2ccc(S(C)(=N)=O)cc2F)c2c(F)c(C3CC3)nn2c1=O ; CC(C)c1cn(Cc2ccc([S@@](C)(=N)=O)cc2F)c2c(F)c(C3CC3)nn2c1=O]

The racemate (150 mg) was separated by Chiral-HPLC with the following conditions (Column: CHIRALPAK-IK, 3*25 mm, 5 μm; Mobile Phase A: Hex (10 mM NH₃-MeOH), Mobile Phase B: ETOH; Flow rate: 40 mL/min; Gradient: isocratic 50; Wave Length: 220/254 nm; RT1 (min): 14.5; Sample Solvent: EtOH: DCM=3:1-HPLC; Number Of Runs: 6), the pure fraction was concentrated and then lyophilized respectively to afford rel-(S)-2-cyclopropyl-3-fluoro-4-(2-fluoro-4-(S-methylsulfonimidoyl)benzyl)-6-isopropylpyrazolo[1,5-a]pyrimidin-7(4H)-one.

ESI-MS m/z=421.10 [M+H]⁺; Calculated: 420.1

¹H NMR (400 MHZ, DMSO-d₆) δ 7.89 (s, 1H), 7.78 (dd, J=9.8, 1.8 Hz, 1H), 7.73 (dd, J=8.0, 1.8 Hz, 1H), 7.38 (t, J=7.7 Hz, 1H), 5.49 (s, 2H), 4.40 (s, 1H), 3.10 (d, J=1.1 Hz, 3H), 2.99 (p, J=6.9 Hz, 1H), 1.95 (tt, J=8.3, 5.0 Hz, 1H), 1.18 (d, J=6.9 Hz, 6H), 1.01-0.95 (m, 2H), 0.90-0.85 (m, 2H).

¹⁹F NMR (377 MHz, DMSO) δ −115.52, −188.47, −188.48.

### Example 2-Biological Assays

### ENPP1 Enzyme Inhibition Assay

### Assay Principle

ENPP1 catalyses the hydrolysis of ATP into AMP+PPi. AMP production was monitored using Bellbrooks Transcreener AMP/GMP TR-FRET assay kit (Cat No. 3020-10K) according to the manufacturer's instructions. The assay was run with 50 mM Tris pH 7.5, 5 mM MgCl₂, 150 mM NaCl, 0.005% Tween20 and distilled H₂O. Antibody and tracer were diluted to 2 nM. Human ENPP1 was run at 20 pM with ATP at 300 nM. Mouse ENPP1 was run at 15 pM with ATP at 400 nM. AMP produced in the assay by ENPP1 activity competes with a far-red tracer labelled AMP bound to an AMP specific antibody resulting in a decrease in TR-FRET. ENPP1 inhibition reduces AMP production and increases the TR-FRET signal.

The pEC₅₀ results are presented in Table 2 below.

### ENPP1 Cellular Assay in MCF-7

### Assay Principle

ENPP1 inhibition was assessed in a human cellular co-culture assay using THP1-Dual interferon reporter cells from InvivoGen (Cat No. thpd-nfis), see Goswami et al., 2022, and MCF-7 cancer cells. MCF-7 cells express ENPP1 which hydrolyses 2′3′-cGAMP added to the media and reduces STING activation and interferon (IFN) response in THP-1 cells. ENPP1 inhibition reduces 2′3′-cGAMP hydrolysis and increases STING activation and IFN response in THP-1 cells.

MCF-7 cells were seeded at 10,000 cells/well in a 96 well plate and 24 h later compounds were added in DMEM+GlutaMAX media+2% Hyclone FBS, with a final DMSO of 0.2%. 2′3′-cGAMP was added at a final concentration of 10 μM and incubated for 30 mins at 37° C. 20,000 THP-1 cells were added per well and incubated for 48 h. IFN response was measured by Quanti-Luc Gold according to the manufacturer's instructions using 15 μL Quanti-Luc Gold per well. Luminescence was measured on an EnSight reader (PerkinElmer).

The pEC₅₀ results are presented in Table 2 below.

### Surface Plasmon Resonance (SPR) Experiments

### Assay Principle

Cytiva Biacore S200, T200 and 3000 machines were used for all SPR experiments. For ENPP1, data were collected at a constant temperature of 10° C. Human ENPP1 was captured on a nickel NTA chip using standard protocols in capture buffer: 50 mM HEPES (pH 7.5), 150 mM NaCl, 50 PM EDTA, 0.05% Tween 20 at ˜12000 RU. The experiments were run in buffer: 50 mM HEPES (pH 7.5), 150 mM NaCl, 5 mM MgCl₂, 0.05% Tween 20, 3% DMSO. For carbonic anhydrases, data were collected at a constant temperature of 25° C. Human CA-I (CA1) or human CA-II (CA2) was captured via standard EDC/NHS protocol on a Biacore CM5 chip using standard protocols in HBS-N buffer (10 mM HEPES (pH 7.4), 150 mM NaCl) at ˜2000 RU. The experiments were run in PBS buffer containing 0.05% Tween 20 and 3% DMSO. All compounds were initially screened at concentrations ranging from 23 nM to 50 UM or 4.6 nM to 10 UM, injecting from the lowest to highest concentrations and adjusting appropriately for each compound for repeat experiments. Scrubber 2 (Biologic software) was used to process and analyse SPR data. Kinetics were fitted using a 1:1 binding model with local Rmax for each concentration where required (see results for dissociation constants KD in Table 2 below). Data for the inhibitors were referenced to those for a blank surface and blank injections to normalise for non-specific binding and drift. A DMSO calibration was run to remove excluded volume effect of binding responses between reference and target surface.

The pKD results are presented in Table 2 below.

TABLE 2
Biochemical data for representative compounds of the disclosure.
pEC₅₀ is the negative logarithm of the EC₅₀ value, where EC₅₀
refers to the concentration of compound required to achieve
50% of its maximal effect. pKD is the negative logarithm
of the equilibrium dissociation constant (KD).

| | ENPP1 | ENPP1 | | | |
|---|---|---|---|---|---|
| | human | human | ENPP1 | CA2 | CA1 |
| COMPOUND | enzyme | cell | human | human | human |
| No. (ref) | pEC₅₀ | pEC₅₀ | SPR pKD | SPR pKD | SPR pKD |
| 1 | 9.4 | 7.9 | 8.7 | <1 | <1 |
| 2 | 6.6 | | | <1 | <1 |
| 3A | 9.5 | 7.9 | 9.8 | <1 | 3.0 |
| 3B | 6.5 | | | <1 | 3.0 |
| 3 Rac | 9.0 | 7.7 | 9.2 | <1 | 3.0 |
| 4 | 9.2 | 7.9 | 8.9 | 4.9 | 3.0 |
| 5 | 9.0 | 7.2 | 9.2 | <1 | <1 |
| 6 | 7.0 | | | 3.6 | 3.0 |
| 7A | 9.0 | 7.8 | 9.1 | <1 | 3.0 |
| 7B | 5.5 | | | <1 | 3.0 |
| 8 | 9.0 | 7.5 | 8.8 | 4.2 | 7.0 |
| 9A | 9.3 | 7.5 | 8.8 | <1 | <1 |
| 9B | 6.8 | | | <1 | <1 |
| 9 Rac | 8.9 | 7.6 | 8.9 | <1 | <1 |
| 10 | 8.9 | 7.3 | 8.3 | 6.4 | 8.2 |
| 11 | 8.8 | 7.5 | 8.1 | 7.1 | 6.8 |
| 12 | 8.1 | 6.0 | | 3.0 | <1 |
| 13 | 8.7 | 7.3 | 8.8 | 6.8 | 5.5 |
| 14 | 8.4 | | | 3.0 | <1 |
| 15 | 8.0 | | 8.5 | 4.2 | 3.9 |
| 16 | 8.6 | 7.2 | 8.8 | 6.0 | 5.4 |
| 17 | 8.5 | | | 7.6 | 6.5 |
| 18 | 8.3 | | 8.7 | 4.4 | 5.3 |
| 19 | 7.3 | | 8.2 | 6.0 | 5.7 |
| 20 | 8.2 | | | <1 | <1 |
| 21 | 8.8 | 7.0 | 8.9 | 5.1 | 4.5 |
| 22 | 8.6 | 7.3 | 8.6 | 6.2 | 7.4 |
| 23 | 8.5 | 6.7 | 8.4 | 6.4 | 6.1 |
| 24 | 8.4 | | 8.5 | 6.6 | 6.9 |
| 25 | 8.2 | | | <1 | <1 |
| 26 | 7.9 | | | 3.0 | 3.0 |
| 27 | 8.2 | 6.6 | | 3.0 | 3.0 |
| 28 | 8.2 | | 8.7 | 6.7 | 6.6 |
| 29 | 8.2 | | 7.6 | 4.5 | 5.1 |
| 30 | 8.4 | 7.1 | | <1 | <1 |
| 31 Rac | 8.2 | 7.2 | | <1 | <1 |
| 31A | 8.4 | 7.5 | | <1 | <1 |
| 31B | 1.0 | | | <1 | <1 |
| 32A | 8.2 | 7.1 | | <1 | <1 |
| 32B | 1.0 | | | <1 | <1 |
| 32 Rac | 8.1 | | | <1 | <1 |
| 33 | 8.1 | | | <1 | <1 |
| 34 | 7.8 | | | 4.1 | 4.0 |
| 35 | 7.7 | | 8.3 | 6.8 | 6.5 |
| 36 | 7.3 | | 8.0 | 4.3 | <1 |
| 37 | 6.2 | | 6.4 | 3.0 | <1 |
| 38 | 7.8 | | 8.6 | 6.4 | 6.1 |
| 39 | 9.8 | 8.8 | 9.2 | 4.1 | <1 |
| 40 | 8.9 | 7.7 | 9.4 | <1 | <1 |
| 41 | 8.8 | 7.4 | 8.8 | 3.0 | 3.0 |
| 42 | 9.1 | 7.7 | 8.8 | 4.5 | 3.0 |
| 43 | 8.4 | | | 4.8 | 3.0 |
| 44 | 8.3 | | | 4.2 | 3.0 |
| 45 | 8.2 | | | 4.4 | <1 |
| 46 | 9.2 | 7.5 | 8.5 | <1 | <1 |
| 47 | 8.8 | 7.1 | 9.0 | <1 | <1 |
| 48 | 7.5 | | | | |
| 49 | 8.5 | 7.3 | 8.5 | <1 | 3.0 |
| 50 | 7.5 | | | | |
| 51 | 8.9 | 7.8 | 9.4 | <1 | <1 |
| 52 | 6.9 | | | | |
| 53 | 7.0 | | | | |
| 54 | 8.9 | 7.4 | | | |
| 55 | 9.5 | 7.6 | | | |
| 56 Rac | 8.6 | | | 4.5 | 3.0 |
| 56A | 9.0 | 7.4 | | | |
| 57 | 9.0 | 7.7 | 9.2 | 3.0 | <1 |
| 58 Rac | 8.7 | | | 3.0 | <1 |
| 58A | 8.9 | 6.7 | | | |
| 59 Rac | 8.2 | | 8.9 | <1 | <1 |
| 59A | 8.1 | 6.5 | 8.6 | <1 | <1 |
| 60 | 8.9 | 7.8 | 9.4 | <1 | <1 |
| 61 | 8.4 | | | 3.8 | 3.0 |
| 62 | 8.2 | | | 3.0 | <1 |
| 63 | 8.0 | | | <1 | <1 |
| 64 | 8.1 | | | | |
| 65 | 8.8 | 7.4 | | 3.0 | 3.0 |
| 66 | 8.9 | | | | |
| 67 | 8.9 | | | | |
| 68 | 8.2 | | | | |
| 69 | 8.4 | 6.9 | | <1 | <1 |
| 70 | 8.5 | 6.9 | | | |
| 71 | 8.5 | 6.6 | | <1 | <1 |
| 72 | 8.6 | 6.7 | | 3.3 | <1 |
| 73 Rac | 8.6 | 6.6 | | | |
| 73A | 8.5 | 6.8 | | | |

### Example 3-In vivo Model 1

### Using Compound 11

It was tested whether ENPP1 could be a druggable target for the development of an alternative treatment for HPP, particularly for the non-lethal later-onset forms of the disease where enzyme replacement is not approved. The Alpl^{Prx1} mouse, as described in Foster et al., 2017 (Foster B L, Kuss P, Yadav M C, Kolli T, Narisawa S, Lukashova L, et al., Conditional Alpl ablation phenocopies dental defects of hypophosphatasia, J Dent Res., 2017, 96 (1), 81-91), was used as a model.

### Methods

### Generation of the Alpl^{Prx1} Model:

Prx1-Cre⁺; Alpl^{ff/fl} mice were generated as described in Foster et al., 2017 and crossed with Alpl^{+/-} mice to generate the Alpl^{Prx1} model.

### Administration of Compound 11 and Collection of Samples:

A powdered base diet was prepared from irradiated standard diet (Teklad Global Diet 2918) by using a grinder (COSUAI, PRC). One group of mice were orally administered with 4 g of the powdered base diet which did not contain Compound 11 (the control group), and one group of mice were orally administered with 4 g of the powdered base diet mixed with Compound 11. The diet was renewed at 3 pm every 24 hours from postnatal day 28 until postnatal day 128 (100 days). Male mice older than 118 days old were given 4.8 g of dosing or control diet as they consumed more than 4 g diet. At day 128, mice were anesthetized with Avertin (0.0017 mL/g body weight) and blood was collected via cardiac puncture and immediately spun down in a heparin tube (BD365987). The plasma was filtrated with a Spin-X column (Coster 8160), frozen down on dry-ice and stored in a −80° freezer until analysis. Bone tissues were fixed in 4% PFA.

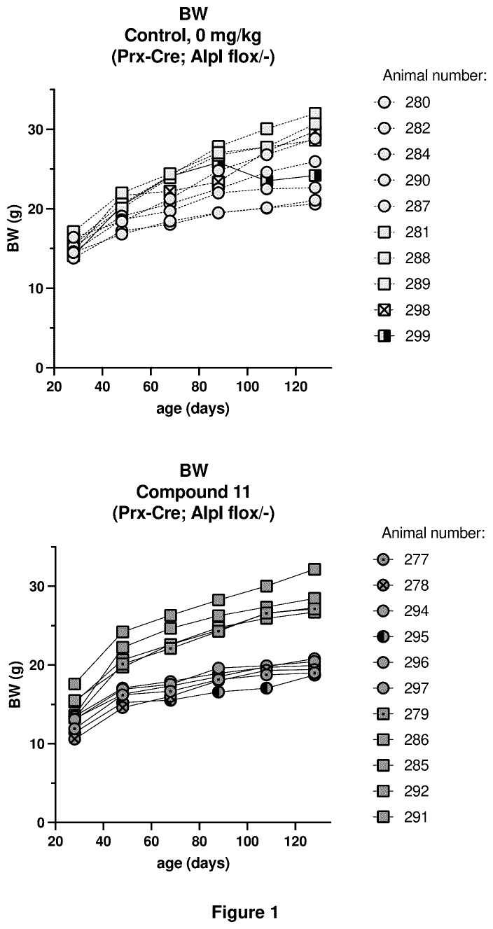

The diet schedule and body weight of the mice as they age is shown in FIG. 1.

### Plasma Analysis:

PPi levels were measured using a standard method involving ATPsulfurylase. Defrosted plasma samples were deproteinated with 10 kD cut off column (Pall OD010C34) and incubated in reaction mixture containing 25 mM Hepes buffer, 1.0 u/mL ATPsulfurylase (R&D, R&D 7175-AS-020), 100 UM adenosine 5′-phossulfate sodium salt (Sigma, A5508), 25 UM MgCl₂ and 25 mM Hepes buffer at 37° C. for 30 min and heated at 94° C. for 5 min to inactivate ATPsulfurylase. ATP converted from PPi in the samples was mixed with BacTiter-Glo (Promega) and luminescence was quantified in duplicated reactions using a iD3X plate reader (Molecular Device). Values obtained from reactions without ATPsulfurylase were subtracted to eliminate ATP which possibly existed in the plasma samples.

Plasma chemistry was performed using Vetscan VS2 analyzer with Comprehensive Diagnostic Profiles rotor (Abaxis). AP levels were measured as we previously published (Narisawa et al, 2013, J Bone Miner Res, 2013 July; 28 (7): 1587-98-In vivo overexpression of tissue-nonspecific alkaline phosphatase increases skeletal mineralization and affects the phosphorylation status of osteopontin). Mouse OPN was measured with Quantikine®-ELISA kit (R&D).

### Imaging Analysis:

Radiographic images were obtained by using XPERT 80-Kubtec Scientific. All images were taken with an energy of 55 kV and 70 μA.

### Bone Morphometry:

PFA fixed bone sections were frozen down and sectioned with Tungsten blade following Kawamoto Film method (Kawamoto T. Use of a new adhesive film for the preparation of multi-purpose fresh-frozen sections from hard tissues, whole-animals, insects and plants. Arch Histol Cytol. 2003; 66 (2): 123-43) and stained with von Kossa and nuclear fast red (Narisawa et al, 2013, J Bone Miner Res, 2013 July; 28 (7): 1587-98-In vivo overexpression of tissue-nonspecific alkaline phosphatase increases skeletal mineralization and affects the phosphorylation status of osteopontin). Three stained sections per mouse were analyzed by Keyence microscope to obtain an average value of mineralized area. Five mice per genotype were used (n=5).

### Results

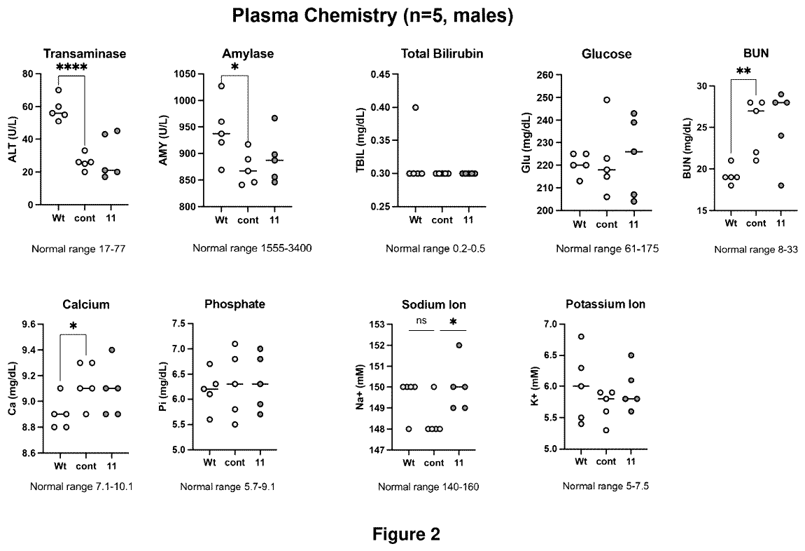

Following analysis of the plasma chemistry of wild-type (WT), control, and Compound 11 treated male mice, the results are shown in FIG. 2. Significant changes in the plasma chemistry were only observed between WT and HPP mice (transaminase, amylase, BUN, and Ca).

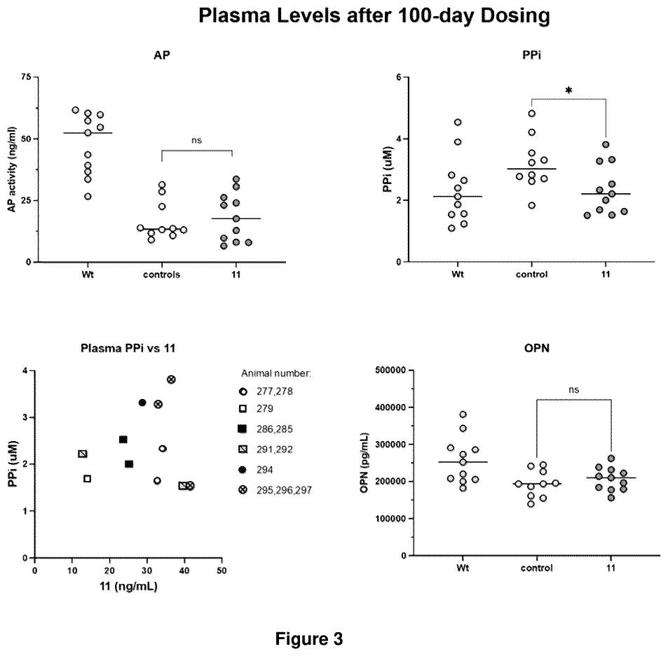

Plasma levels of AP, OPN and PPi (including plasma PPi vs. Compound 11) after 100 days of dosing were also analysed. The results are shown in FIG. 3. Administration of Compound 11 did not affect the AP and OPN levels; however, it can be seen that PPi levels in the dosed animals were reduced to the levels of WT mice.

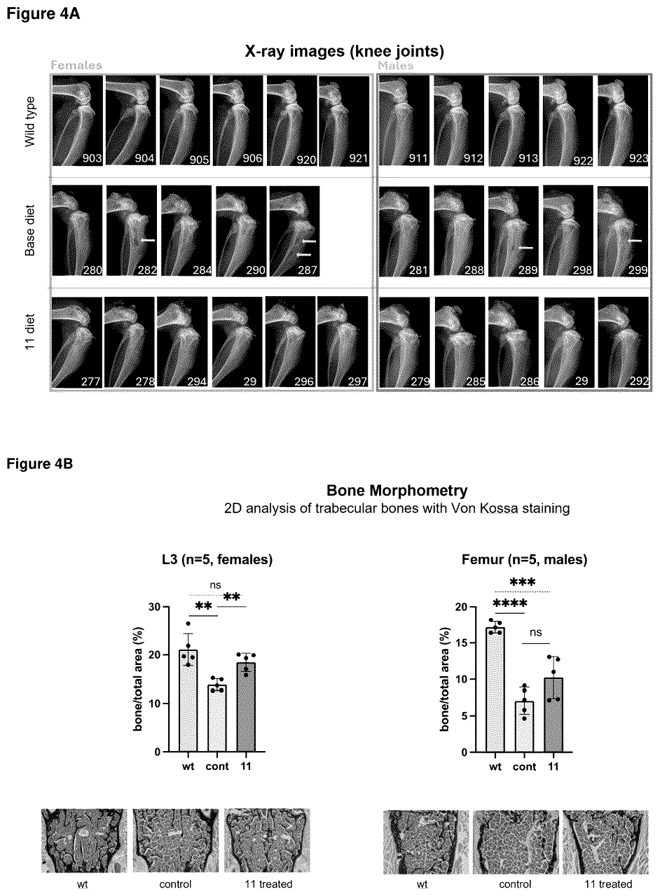

X-rays of the knee joints of both male and female mice were taken and a bone morphometric analysis (2D analysis of trabecular bones) was performed on Von Kossa-stained sections using Kawamoto's film method (Kawamoto T. Use of a new adhesive film for the preparation of multi-purpose fresh-frozen sections from hard tissues, whole-animals, insects and plants. Arch Histol Cytol. 2003; 66 (2): 123-43). The results of the X-rays and bone morphometric analysis are shown in FIGS. 4A and 4B, respectively.

The X-ray images in FIG. 4A show darker areas in the tibia of untreated HPP mice, indicating lesser mineralization. The incidence of those darker areas was reduced in treated animals. This suggests that treatment with Compound 11 increased bone mineralization of the bone cortex.

FIG. 4B shows that in the L3 vertebrate bone of dosed HPP animals, mineralization was increased to the levels of wild-type mice. In the trabecular bones of the distal femur, HPP mice with Compound 11 treatment showed slight improvement in mineralization.

### Using Other Compounds of the Invention

The in vivo experiments outlined above were also conducted using other compounds of the invention, subject to the following changes to the bone morphology experiments:

The Kawamoto Film method used was as outlined in the following reference: Kawamoto T, Kawamoto K. Preparation of thin frozen sections from nonfixed and undecalcified hard tissues using Kawamot's film method (2012). Methods Mol Biol. 2014; 1130:149-164. In addition, instead of five mice per genotype, a minimum of three mice per condition were used (e.g. n=3). Furthermore, additional sections were stained with Goldner's trichrome by following a standard protocol using three solutions of Ponceau Acid Fuchsin, Phosphomolybdic Acid-Orange G, and Light Green.

### Example 4—In Vivo Model 2

### Methods

### Generation of the Alp^{-/-}-Model:

An Alpl knockout (KO) mouse model was constructed by knocking out exons 3-4 of the Alpl gene in the C57BL/6J strain using CRISPR-Cas9 technology. In vitro fertilization (IVF) and gestational surrogacy using female CD-1 mice was performed to expand the colony and generate mice with the desired genotypes.

Genotyping of WT, Alpl^{+/-}, and Alpl^{-/-} mice:

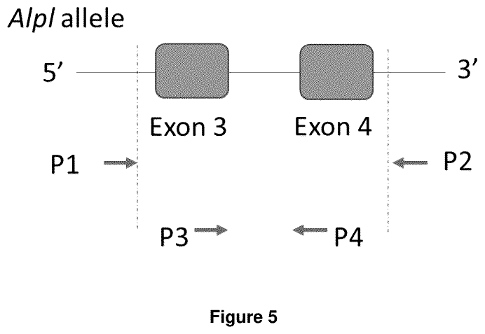

At postnatal day 0 (P0), toe clipping of the pups was performed to extract DNA, and genotyping was conducted using the two pairs of primer and PCR method shown in FIG. 5.

The expected sizes of the PCR products for different genotypes are as follows:

Length of PCR product

Primer pair

| Genotype | P1 and P2 | P3 and P4 |
|---|---|---|
| WT | 1837 bp* | 912 bp |
| Alpl^{+/-} | 716 bp, 1837 bp* | 912 bp |
| Alpl^{-/-} | 716 bp | None |

*The large PCR product (1837 bp) may not show robust signals in some cases. The genotypes are confirmed with both PCR results using two different sets of primer pairs. For example, The Alpl^{-/-} genotype is always confirmed with the 716 bp PCR product with P1/P2 primers in combination with no PCR product with P3/P4 primers.

Primer sequences

| Primer | Sequence (5′→3′) | Primer Type | SEQ ID NO |
|---|---|---|---|
| P1 | TGCCTTCCCCACTACCCTAACAA | Forward | 1 |
| P2 | TACACTCCCCTCCCACTCCACTAT | Reverse | 2 |
| P3 | GGGCTCCTTGGGTAATGTATCC | Forward | 3 |
| P4 | ATTCTCAGGGCGGTAGGTTAGTCC | Reverse | 4 |

### Animal Care and Diet:

All pups should stay with their mother before P21 (WT) or P29 (homozygous) until weaning, and all pregnant and nursing CD-1 mice and all pups should be fed with VB6 supplemented diet during the whole experiment (VB6 concentration: 300 mg/kg). Animals have free access to irradiation sterilized dry granule food during the entire study period and free access to sterile drinking water.

### Administration of Compounds and Collection of Samples:

Starting from postnatal day 1 (P1), compounds are administered subcutaneously once daily.

For plasma PPi measurements, blood samples are collected through submandibular venipuncture into 1.5 mL commercial heparin lithium anticoagulant tubes at predetermined time points post treatment. The samples are centrifuged at 2,500 g at 4° C. for 15 min to collect the plasma. The plasma is centrifuged and filtered with a Centrifugal Concentrator (Spin-X column 0.22 uM, Coster 8160) to gain platelet-free plasma, which is stored at −80° C. prior to analysis.

For drug level (PK) measurement, blood samples are collected through submandibular venipuncture into 1.5 mL commercial EDTA anticoagulant tubes (KANGJIAN, 0440272) at predetermined time points post treatment. The samples are centrifuged at 2,500 g at 4° C. for 15 min to collect the plasma, which is stored at −80° C. before drug level measurement.

For ALP activity measurement, blood samples are collected from mice through submandibular venipuncture into 1.5 mL tubes at predetermined time points post treatment. The blood samples are placed at room temperature for 30 min, and then centrifuged at 12,000 rpm at 4° C. for 30 min. The resulting serum is stored at −80° C. before testing.

Bone tissues are collected at the end of the study or when the animals are found dead and stored in formalin at room temperature before further analysis.

### ALP Activity Assay:

The ALP activity assay is performed according to the protocol of a commercial kit (Beyotime, P0321S) with some modifications. The substrate solution, p-nitrophenyl phosphate (pNPP) solution, and standard solutions of p-nitrophenol (pNP) are prepared using DEA Assay Buffer (diethanolamine, pH=9.8). The reaction mix is set up in 96-well plates for blank controls, standards, and samples. Absorbance at 405 nm (OD405) is measured continuously at 37° C. for 5 minutes at intervals of 30 seconds. One unit of ALP activity is defined as the amount of ALP required to hydrolyze 1 micromole of p-nitrophenyl phosphate (pNPP) to p-nitrophenol (pNP) per minute at pH 9.8 and a temperature of 37° C. in the diethanolamine (DEA) buffer.

### Hanging Time Test:

The mice are allowed to adjust to the testing room for at least 30 minutes prior to testing. Mice are placed on a wire grid. The grid is inverted so that the mouse is hanging upside down. Measurement: start the timer as soon as the mouse is hanging on the wire. Stop the timer when the mouse falls onto the soft material below, a cut-off time (60 seconds) is employed. Allow the mouse to rest for 20 min to avoid fatigue. Repeat 3 times, each time with a rest period in between. Record the hang time for each trial. Calculate the average of all trials for each mouse.

### Open Field Test:

The open field arena (LE 8825 IR MOTOR ACTIVITY MONITOR, Panlab) is cleaned thoroughly to remove any scents or residues from previous tests. The mice are acclimated to the testing environment by placing them in the testing room for at least 30 minutes before the test and then are placed gently in the centre of the open field arena. The mice are allowed to explore the open field for a fixed duration, typically 10 minutes. After the test period, mice are gently removed from the arena and returned to their home cage. The open field arena is cleaned thoroughly before testing the next mouse to avoid scent cues affecting behaviour.

### X-Ray Imaging Analysis:

Mice or bone samples are placed in the right position in the calibrated system (Start INSIGHT VET DXA, OsteoSys, Korea) to ensure the regions of interest are centred in the scanning field. The scanning is initiated using energy of 26 KV and an exposure time of 10 s. The built-in image analysis tools of the system are used to delineate the bone tissue of the whole body or specific bone regions, and output the corresponding values including BMC, BMD, Fat (%) and Lean (%).

### Micro-CT:

Fixed femora and vertebrae were imaged (Jiangsu Huajing Molecular Imaging and Drug Research Institute, Suzhou, China, http://www.hjmd.cc/) with a Micro-PET/CT scanner (Inveon Micro-PET/CT, SIEMENS). Two-dimensional imaging data were acquired using IAW (Inveon Acquisition Workplace 2.1.272) with parameters of 80 kV/200 μA, 49.8 ms exposure, and 0 mm filter. Acquired imaging data were reconstructed into 2D virtual stack images using IAW (Inveon Acquisition Workplace 2.1.272). The calibration phantoms are water and air. The water phantom is scanned and reconstructed using the same parameters as the bone samples to correct for bone density. Data analysis was performed using the General Analysis and Multimodal 3D Visualization modules in IRW (Inveon Research Workplace).

### Bone Histopathology:

Non-decalcified bone samples were fixed in 10% neutral buffered formalin, dehydrated in graded concentrations of ethanol and embedded in methylmethacrylate. Embedded bones were sectioned using a hard tissue cutting machine (EXAKT Technologies) and stained with Goldner's Trichrome to examine bone mineralization.

### Plasma Analysis:

PPi levels are measured using a standard method involving ATP sulfurylase. Defrosted plasma samples are deproteinated with 10 kD cut off column (Amicon UFC501024) and incubated in reaction mixture containing 50 mM Hepes buffer, 5 μg/mL ATPsulfurylase (R&D, R&D 7175-AS-020), 5 μM adenosine 5′-phosphosulfate sodium salt (Cayman 21226), 80 uM MgSO₄ at 37° C. for 45 min and heated at 95° C. for 10 min to inactivate ATPsulfurylase. ATP converted from PPi in the samples was mixed with BacTiter-Glo (Promega) and luminescence was quantified in duplicated reactions using an EnSight™ Multimode Plate Reader (PerkinElmer). Values obtained from reactions without ATP sulfurylase were subtracted to eliminate ATP which possibly existed in the plasma samples.

Modifications may be made to the above examples without departing from the scope of the present disclosure as defined in the accompanying clauses and claims.

Alternative expressions of the inventive concept are set out in each of the following numbered clauses:

### CLAUSES

1. A compound of formula (I) or a pharmaceutically acceptable salt or solvate thereof:

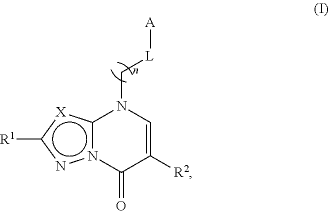

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having one or more (e.g. 1, 2, 3, or 4) ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   L is selected from phenyl, 6-membered heteroaryl having one or more (e.g. 1, 2, 3, or 4) ring heteroatoms independently selected from N, O, and S, C3-C7 cycloalkyl, 3-7-membered heterocycloalkyl having one or more (e.g. 1, 2, 3, or 4) ring heteroatoms independently selected from N, O, and S, C9-C10 bicyclic aryl, and 9-10-membered bicyclic heteroaryl having one or more (e.g. 1, 2, 3, or 4) ring heteroatoms independently selected from N, O, and S, wherein each phenyl, heteroaryl, cycloalkyl, heterocycloalkyl, bicyclic aryl or bicyclic heteroaryl is optionally substituted with one or more (e.g., 1, 2, 3, 4, or 5) substituents independently selected from R6 and R7;
   each R6 and R7 are independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me;
   each R11 is independently selected from H and C1-C3 alkyl;
   A is selected from —SO₂NH₂ and —S(O)(NH)R13;
   R13 is C1-C3 alkyl, C1-C3 haloalkyl, or C3-C6 cycloalkyl, wherein cycloalkyl is optionally substituted with one or more (e.g., 1, 2, 3, or 4) halo; or
   when L is phenyl or heteroaryl, R13 and R6 or R7 together form a 5-6 membered heterocycloalkyl optionally having one or more (e.g., 1, 2, or 3) additional ring heteroatoms independently selected from N, S and O.

2. The compound of Clause 1, wherein X is N.

3. The compound of Clause 1, wherein X is C(CH₂)ₘR5, and R5 is as defined in Clause 1.

3a. The compound of Clause 1 or Clause 3, wherein m is 0.

3b. The compound of Clause 1 or Clause 3, wherein m is 1.

4. The compound of any of Clauses 1, 3 or 3a, wherein X is CH.

5. The compound of any of Clauses 1, 3 or 3a, wherein X is C-halo, optionally wherein halo is Cl or F (e.g. F).

6. The compound of any of Clauses 1, 3 or 3a, wherein X is CR5, and R5 is OMe.

7. The compound of any of Clauses 1, 3 or 3a, wherein X is CR5, and R5 is CN.

8. The compound of any of Clauses 1, 3 or 3a, wherein X is CR5, and R5 is Me.

9. The compound of any of Clauses 1, 3, 3a or 4, wherein X is CR5, and R5 is 2H.

9a. The compound of any of Clauses 1, 3, 3a or 4, wherein X is CR5, and R5 is 1H,

10. The compound of any of Clauses 1 to 9a, wherein R1 is H or halo.

11. The compound of any of Clauses 1 to 9a, wherein R1 is C1-C6 alkyl or C1-C6 haloalkyl.

11a. The compound of any of Clauses 1 to 9a or 11, wherein R1 is C1-C6 alkyl.

12. The compound of any of Clauses 1 to 9a, wherein R1 is C3-C6 cycloalkyl (e.g. cyclopropyl), optionally substituted with one or more (e.g., 1, 2, 3, or 4) substituents independently selected from C1-C3 alkyl, C3-C6 cycloalkyl and halo.

12a. The compound of any of Clauses 1 to 9a or 12, wherein R1 is C3-C6 cycloalkyl.

12b. The compound of any of Clauses 1 to 9a, 12 or 12a, wherein R1 is cyclopropyl.

12c. The compound of any of Clauses 1 to 9a, wherein R1 is cyclopropyl or ethyl.

13. The compound of any of Clauses 1 to 9a, wherein R1 is C1-C6 alkoxyl or C1-C6 haloalkoxyl.

13a. The compound of any of Clauses 1 to 9a or 13, wherein R1 is C1-C6 alkoxyl.

13b. The compound of any of Clauses 1 to 10, wherein R1 is H.

14. The compound of any of Clauses 1 to 9a, wherein R1 is H or is selected from:

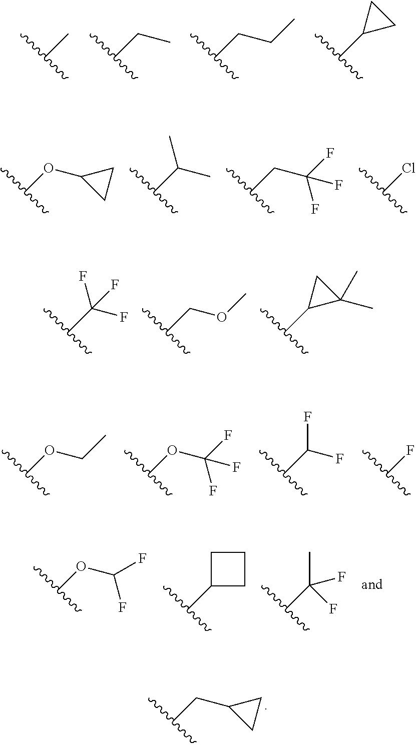

15. The compound of any of Clauses 1 to 9a or 14, wherein R1 is H or is selected from:

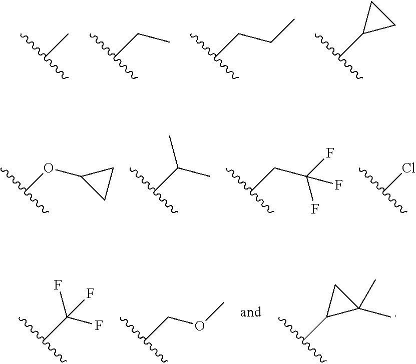

16. The compound of any of Clauses 1 or 3 to 3b, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

17. The compound of any of Clauses 1, 3 to 3b or 16, wherein R5 and R1 taken together with the atoms to which they are attached form a 5-6 membered cycloalkyl.

18. The compound of any of Clauses 1, 3 to 3b or 16, wherein R5 and R1 taken together with the atoms to which they are attached form a 5-6 membered heterocycloalkyl having one or more (e.g. 1 or 2) ring heteroatoms independently selected from N, O, and S.

19. The compound of any of Clauses 1, 3 to 3b or 16, wherein R5 and R1 taken together with the atoms to which they are attached form a phenyl, optionally substituted with one or more (e.g., 1, 2, 3, or 4) C1-C6 alkoxyl.

20. The compound of any of Clauses 1, 3 to 3b or 16, wherein R5 and R1 taken together with the atoms to which they are attached form a 5-6 membered heteroaromatic ring having one or more (e.g. 1 or 2) ring heteroatoms independently selected from N, O, and S.

21. The compound of any of Clauses 1, 3 to 3b or 16, wherein the compound is selected from the following structures:

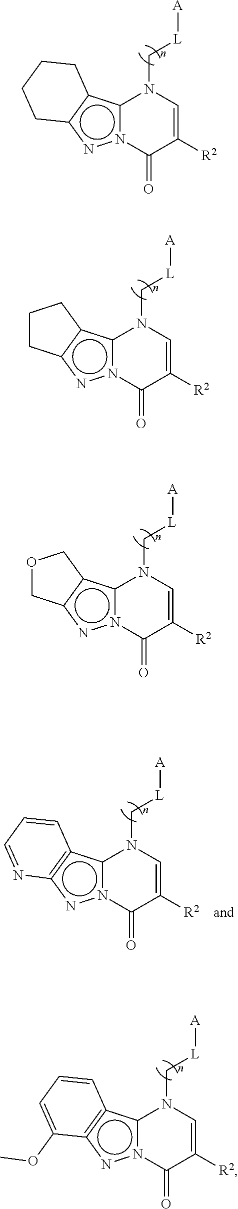

wherein R2, n, L and A are as defined in Clause 1.

22. The compound of any of Clauses 1 to 21, wherein R2 is C1-C6 alkyl or C1-C6 haloalkyl.

22a. The compound of any of Clauses 1 to 22, wherein R2 is C1-C6 alkyl.

22b. The compound of any of Clauses 1 to 22a, wherein R2 is C1-C3 alkyl.

22c. The compound of any of Clauses 1 to 22b, wherein R2 is isopropyl.

23. The compound of any of Clauses 1 to 21, wherein R2 is C3-C6 cycloalkyl, optionally substituted with one or more (e.g., 1, 2, 3, or 4) halo.

24. The compound of any of Clauses 1 to 21, wherein R2 is phenyl, optionally substituted with one or more (e.g., 1, 2, 3, or 4) halo.

25. The compound of any of Clauses 1 to 21, wherein R2 is 6-membered heteroaryl having one or more (e.g. 1, 2, 3, or 4) ring heteroatoms independently selected from N, O, and S, wherein the heteroaryl is optionally substituted with one or more (e.g., 1, 2, 3, or 4) halo.

26. The compound of any of Clauses 1 to 21, wherein R2 is selected from:

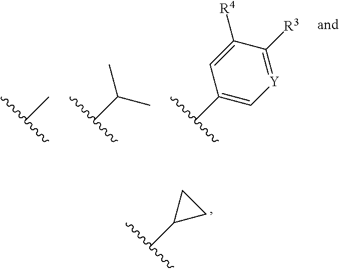

wherein Y is CH or N; R3 is H or halo; and R4 is H or halo (optionally halo is F).

27. The compound of any of Clauses 1 to 21 or 26, wherein R2 is selected from:

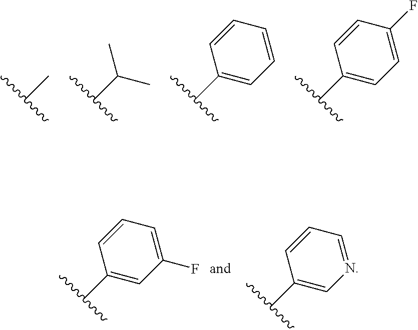

28. The compound of any of Clauses 1 to 27, wherein L is selected from phenyl and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from R6 and R7.

28a. The compound of any of Clauses 1 to 28, wherein L is phenyl, and the phenyl is optionally substituted with one or more (e.g., 1, 2, 3, or 4) substituents independently selected from R6 and R7.

28b. The compound of any of Clauses 1 to 28a, wherein L is phenyl, and the phenyl is optionally substituted with one or more (e.g., 1, 2, 3, or 4) substituents independently selected from C1-C3 alkyl and halo (e.g. F).

28c. The compound of any of Clauses 1 to 28b, wherein L is phenyl, and the phenyl is optionally substituted with one or more (e.g., 1, 2, 3, or 4) substituents independently selected from methyl and halo (e.g. F).

29. The compound of any of Clauses 1 to 27, wherein L is selected from C3-C7 cycloalkyl and 3-7-membered heterocycloalkyl having one or more (e.g. 1, 2, 3, or 4) ring heteroatoms independently selected from N, O, and S, wherein each cycloalkyl or heterocycloalkyl is optionally substituted with one or more (e.g., 1, 2, 3, 4, or 5) substituents independently selected from R6 and R7.

30. The compound of any of Clauses 1 to 27, wherein L is selected from C9-C10 bicyclic aryl and 9-10-membered bicyclic heteroaryl having one or more (e.g. 1, 2, 3, or 4) ring heteroatoms independently selected from N, O, and S, wherein each bicyclic aryl or bicyclic heteroaryl is optionally substituted with one or more (e.g., 1, 2, 3, 4, or 5) substituents independently selected from R6 and R7.

31. The compound of any of Clauses 1 to 27, wherein L-A is selected from;

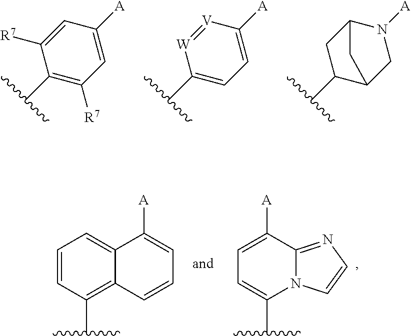

wherein
   V is CR6 or N;
   W is CR7 or N; and
   each R6 and R7 is independently selected from H, halo, hydroxy, C1-C4 alkoxy, C1-C4 haloalkoxyl, C1-C6 alkyl, C1-C6 haloalkyl, C1-C3 alkoxyl-C1-C3 alkyl, phenyl, 6-membered heteroaryl having one or more (e.g. 1, 2, 3, or 4) ring heteroatoms independently selected from N, O, and S, —NH₂, —NHMe, and —NMe₂, wherein each phenyl or heteroaryl is optionally substituted with one or more (e.g., 1, 2, 3, or 4) halo.

32. The compound of any of Clauses 1 to 27 or 31, wherein L-A is selected from:

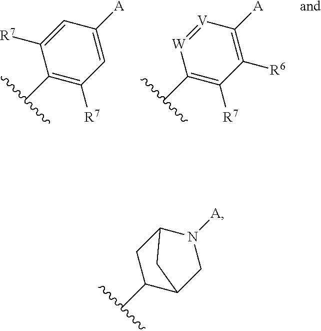

wherein
V is CR6 or N;
W is CR7 or N; and
each R6 and R7 is independently selected from H, halo, hydroxy, C1-C4 alkoxy, C1-C4 haloalkoxyl, C1-C6 alkyl, C1-C6 haloalkyl, C1-C3 alkoxyl-C1-C3 alkyl, phenyl, 6-membered heteroaryl having one or more (e.g. 1, 2, 3, or 4) ring heteroatoms independently selected from N, O, and S, —NH₂, —NHMe, and —NMe₂, wherein each phenyl or heteroaryl is optionally substituted with one or more (e.g., 1, 2, 3, or 4) halo.

32a. The compound of any of Clauses 1 to 27, 31 or 32, wherein L-A is selected from:

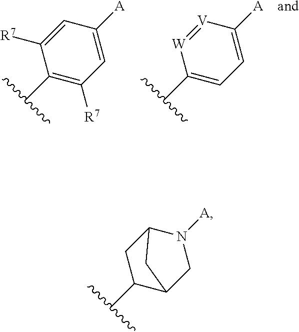

wherein
V is CR6 or N;
W is CR7 or N; and each R6 and R7 is independently selected from H, halo, hydroxy, C1-C4 alkoxy, C1-C4 haloalkoxyl, C1-C6 alkyl, C1-C6 haloalkyl, C1-C3 alkoxyl-C1-C3 alkyl, phenyl, 6-membered heteroaryl having one or more (e.g. 1, 2, 3, or 4) ring heteroatoms independently selected from N, O, and S, —NH₂, —NHMe, and —NMe₂, wherein each phenyl or heteroaryl is optionally substituted with one or more (e.g., 1, 2, 3, or 4) halo.

33. The compound of any of Clauses 1 to 28a, 29 or 30, wherein R6 is selected from H, halo, hydroxy, C1-C4 alkoxyl, C1-C4 haloalkoxyl, C1-C6 alkyl, C1-C6 haloalkyl, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me.

34. The compound of any of Clauses 1 to 28a or 29 to 33, wherein R7 is selected from H, halo, C1-C4 alkyl, hydroxy and —NHMe.

35. The compound of any of Clauses 1 to 28a or 29 to 34, wherein R7 is selected from H, halo, —NHMe and hydroxy, optionally wherein halo is F or Cl.

35a. The compound of any of Clauses 1 to 28b or 29 to 34, wherein R7 is selected from H, halo (e.g. F) and C1-C3 alkyl.

35b. The compound of any of Clauses 1 to 34 or 35a, wherein R7 is selected from H, halo (e.g. F) and methyl.

36. The compound of any of Clauses 1 to 28a or 29 to 33, wherein R7 is NHMe, NH₂ or NMe₂.

36a. The compound of any of Clauses 1 to 28a, 29 to 32a or 34 to 36, wherein R6 is NHMe, NH₂ or NMe₂.

37. The compound of any of Clauses 1 to 36, wherein R6 is H.

37a. The compound of any of Clauses 1 to 28b or 29 to 36, wherein R6 is selected from H, halo (e.g. F) and C1-C3 alkyl.

37b. The compound of any of Clauses 1 to 36 or 37a, wherein R6 is selected from H, halo (e.g. F) and methyl.

38. The compound of any of Clauses 1 to 35b or 36a to 37b, wherein R7 is H.

39. The compound of any of Clauses 31 to 38, wherein Vis N.

40. The compound of any of Clauses 31 to 39, wherein W is N.

41. The compound of any of Clauses 1 to 28a, 29 to 36 or 38 to 40, wherein R6 is H or is selected from:

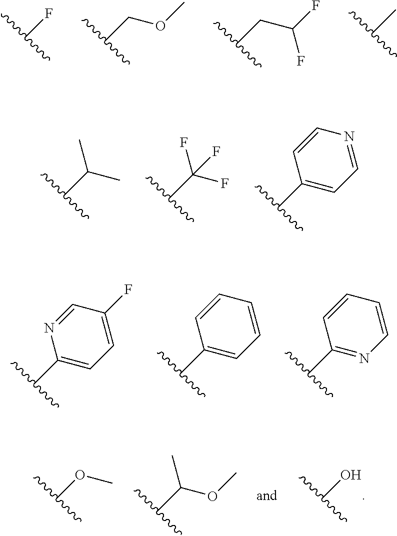

42. The compound of any of Clauses 1 to 41, wherein L-A is:

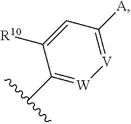

wherein
V is N or CR6;
W is N or CR7;
R6 is H, halo, hydroxy, C1-C4 alkyl, C1-C4 haloalkyl, C1-C4 alkoxyl, C1-C4 haloalkoxyl, C1-C3 alkoxyl-C1-C3 alkyl, phenyl, or 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R7 is H, C1-C4 alkyl, hydroxy, —N(H)(R11), or halo;
R10 is H or halo; and
R11 is H or C1-C3 alkyl.

43. The compound of Clause 42, wherein V is N.

44. The compound of Clause 42, wherein V is CR6, and R6 is selected from H, C1-C4 alkyl, C1-C4 haloalkyl, C1-C4 alkoxyl, C1-C4 haloalkoxyl, phenyl, and 6-membered heteroaryl having 1, 2 or 3 ring heteroatoms independently selected from N, O, and S, wherein each phenyl or heteroaryl is optionally substituted with one or more (e.g., 1, 2, 3, or 4) halo.

45. The compound of any of Clauses 42 to 44, wherein W is N.

46. The compound of any of Clauses 42 to 44, wherein W is CR7, and R7 is selected from H, C1-C4 alkyl, hydroxy, —N(H)(R11), and halo.

47. The compound of any of Clauses 42 to 46, wherein R10 is H.

48. The compound of any of Clauses 42 to 46, wherein R10 is halo.

49. The compound of any of Clauses 42 to 48, wherein R11 is H.

50. The compound of any of Clauses 42 to 48, wherein R11 is C1-C3 alkyl (e.g. Me).

51. The compound of any of Clauses 42 to 50, wherein L-A is selected from:

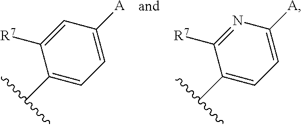

and R7 is independently selected from H, halo, hydroxy, C1-C4 alkoxyl, C1-C4 haloalkoxyl, C1-C6 alkyl, C1-C6 haloalkyl, —N(H)R11, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me, and R11 is as defined in any of Clauses 42, 49 or 50.

52. The compound of any of Clauses 1 to 50, wherein L-A is selected from:

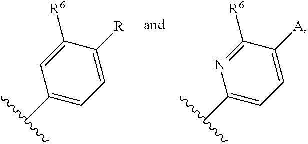

and R6 is independently selected from H, halo, hydroxy, C1-C4 alkoxyl, C1-C4 haloalkoxyl, C1-C6 alkyl, C1-C6 haloalkyl, —N(H)R11, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me, and R11 is as defined in any of Clauses 42, 49 or 50.

53. The compound of any of Clauses 1 to 52, wherein when L is phenyl or 6-membered heteroaryl having one or more (e.g. 1, 2, 3, or 4) ring heteroatoms independently selected from N, O, and S, R13 and R6 may be taken together to form a 5-6 membered heterocycloalkyl optionally having one or more (e.g. 1, 2, 3, or 4) additional heteroatoms selected from N, S and O.

54. The compound of any of Clauses 1 to 27, wherein L-A is selected from:

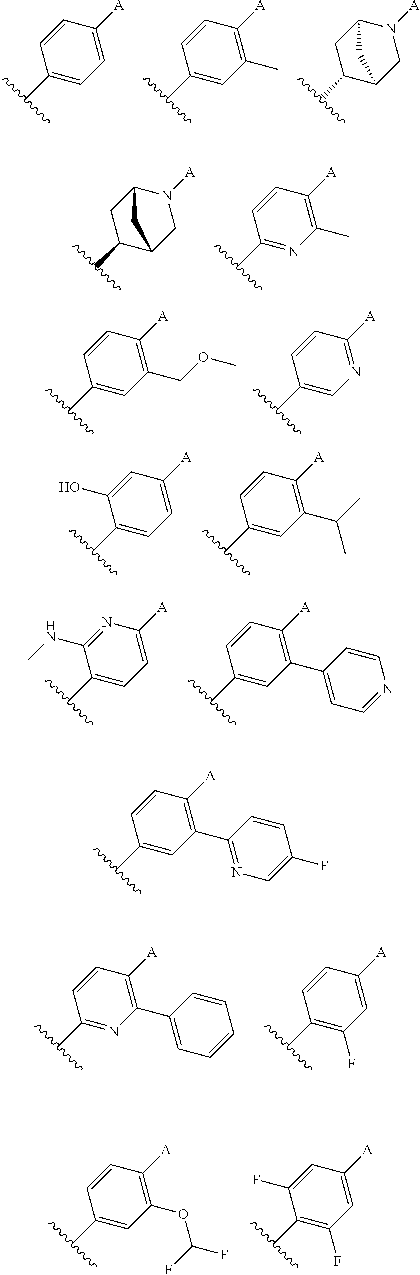

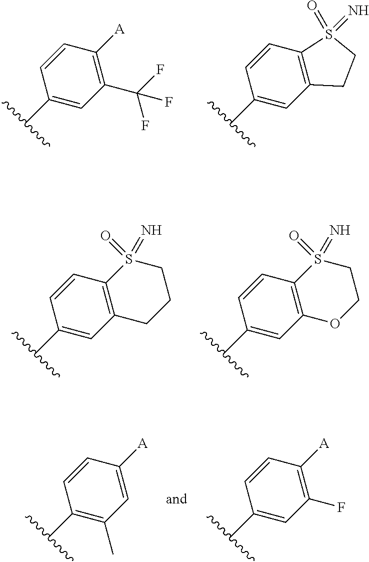

55. The compound of any of Clauses 1 to 54, wherein L-A is selected from:

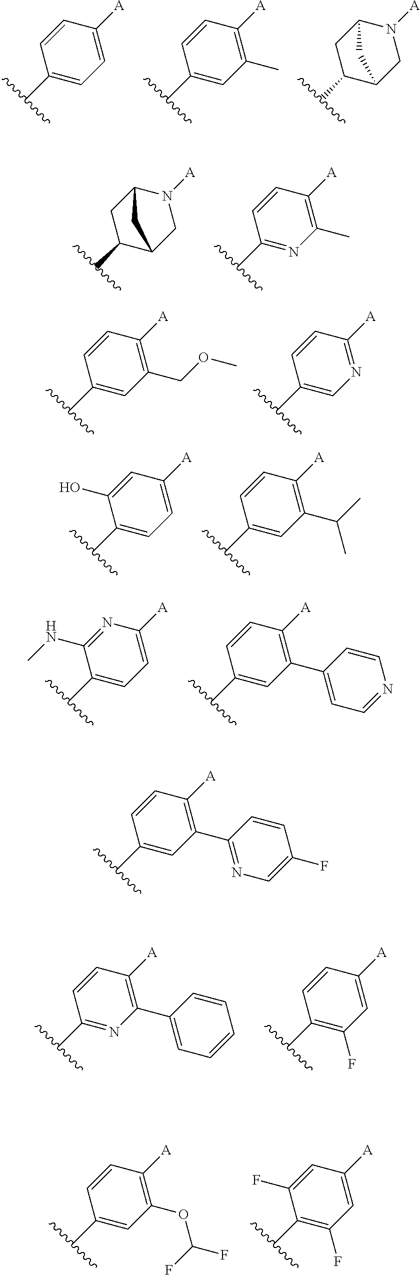

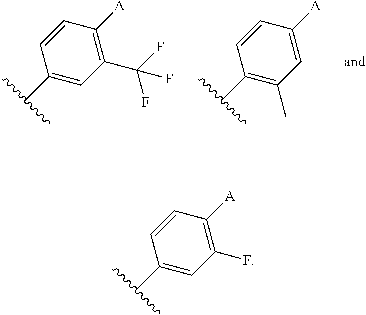

55a. The compound of any of Clauses 1 to 55, wherein L-A is selected from:

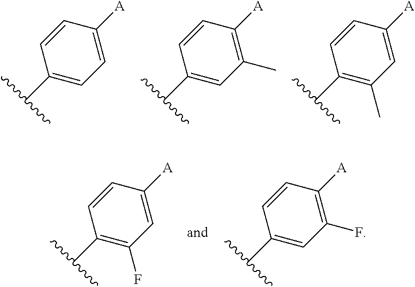

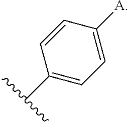

55b. The compound of any of Clauses 1 to 55a, wherein L-A is

56. The compound of any of Clauses 1 to 27, wherein L-A is selected from:

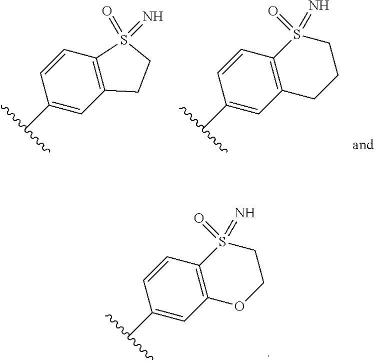 [SMILES: *c1ccc2c(c1)CCS2(=N)=O ; *c1ccc2c(c1)CCCS2(=N)=O ; *c1ccc2c(c1)OCCS2(=N)=O]

57. The compound of any of Clauses 1 to 55, wherein L-A is selected from:

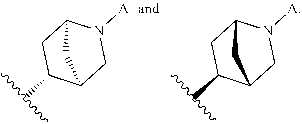

58. The compound of any of Clauses 1 to 55, wherein L-A is selected from.

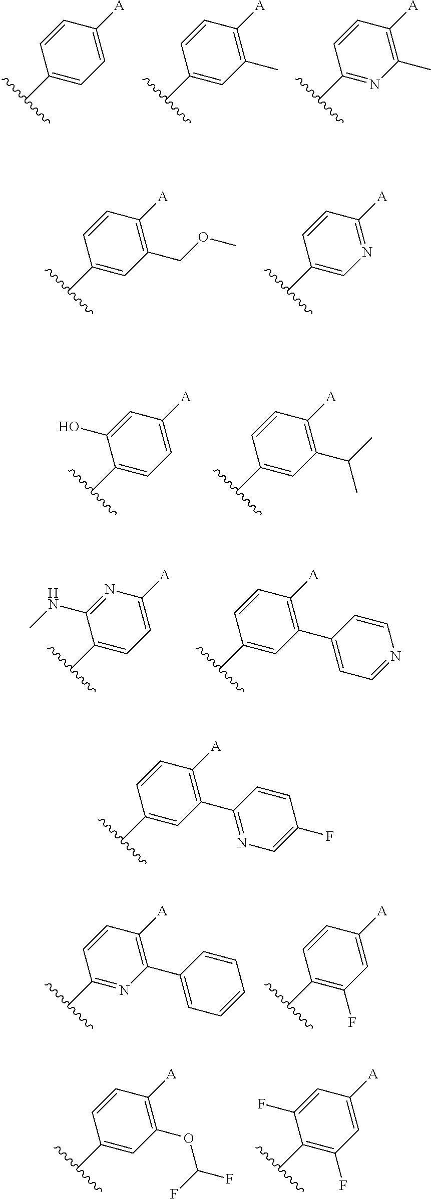

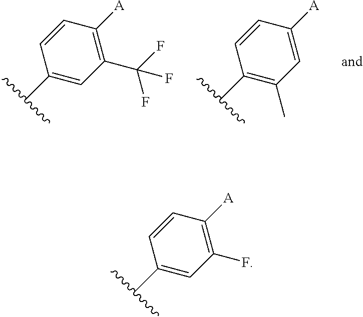

59. The compound of any of Clauses 1 to 58, wherein n is 1.

60. The compound of any of Clauses 1 to 58, wherein n is 2.

61, The compound of any of Clauses 1 to 58, wherein n is 3.

62. The compound of any of Clauses 1 to 61, wherein A is —SO₂NH₂.

63. The compound of any of Clauses 1 to 61, wherein A is —S(O)(NH)R13, wherein R13 is C1-C3 alkyl (e.g. C¹H₃ or C²H₃), C1-C3 haloalkyl, or C3-C6 cycloalkyl, and cycloalkyl is optionally substituted with one or more (e.g., 1, 2, 3, or 4) halo.

64. The compound of any of Clauses 1 to 61 or 63, wherein R13 is C1-C3 alkyl (e.g. C₁H³ or C²H₃) or C1-C3 haloalkyl (e.g. CF₃ or CHF₂).

64a. The compound of any of Clauses 1 to 61, 63 or 64, wherein R13 is Me (e.g. C₁H³ or C²H₃).

65. The compound of any of Clauses 1 to 61 or 63, wherein R13 is C3-C6 cycloalkyl (e.g. cyclopropyl), optionally substituted with one or more (e.g., 1, 2, 3, or 4) halo.

66. The compound of any of Clauses 1 to 61, 63 or 65, wherein R13 is C3-C6 cycloalkyl.

66a. The compound of any of Clauses 1 to 61, 63, 65 or 66, wherein R13 is cyclopropyl.

66b. The compound of any of Clauses 1 to 61 or 63, wherein R13 is selected from C3-C6 cycloalkyl and C1-C3 alkyl.

66c. The compound of any of Clauses 1 to 61, 63 or 66b, wherein R13 is selected from cyclopropyl, methyl (e.g. C¹H₃ or C²H₃), ethyl and isopropyl.

67. The compound of any of Clauses 1 to 61 or 63 wherein A is selected from:

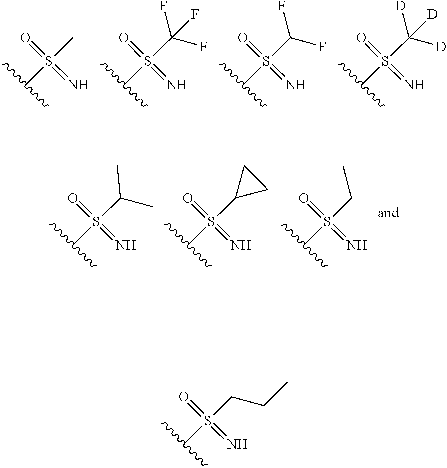

67a. The compound of any of Clauses 1 to 61, 63 or 67, wherein A is selected from:

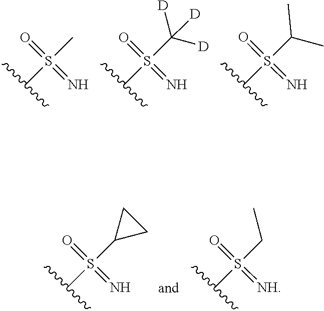

68. The compound of any of Clauses 1 to 61, 63 to 64a or 66b to 67a, wherein A is —S(O)(NH)Me.

69. The compound of any of Clauses 1 to 62, wherein A is selected from:

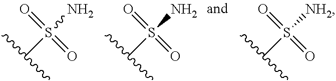

wherein [symbol] indicates a racemic mixture.

70. The compound of any of Clauses 1 to 62 or 69 wherein A is:

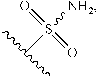 [SMILES: *S(N)(=O)=O]

wherein [symbol] indicates a racemic mixture.

71. The compound of any of Clauses 1 to 62 or 69, wherein A is selected from:

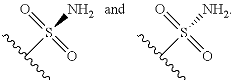

72. The compound of any of Clauses 1 to 61 or 63 to 68, wherein A is selected from:

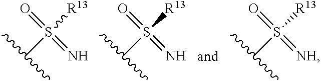

wherein [symbol] indicates a racemic mixture and wherein R13 is as defined in any of Clauses 1 or 63 to 66c.

73. The compound of any of Clauses 1 to 61, 63 to 68 or 72, wherein A is:

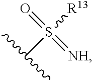

wherein [symbol] indicates a racemic mixture and wherein R13 is as defined in any of Clauses 1 or 63 to 66c.

74. The compound of any of Clauses 1 to 61, 63 to 68 or 72, wherein A is selected from:

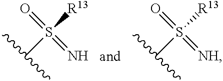

and wherein R13 is as defined in any of Clauses 1 or 63 to 66c.

75. The compound of any of Clauses 1 to 61, 63 to 68 or 72 to 74, wherein A is selected from:

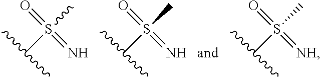

wherein [symbol] indicates a racemic mixture.

76. The compound of any of Clauses 1 to 61, 63 to 68 or 72 to 75, wherein A is:

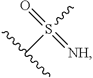 [SMILES: *S(C)(=N)=O]

wherein [symbol] indicates a racemic mixture.

77. The compound of any of Clauses 1 to 61, 63 to 68 or 72 to 75, wherein A is selected from:

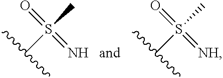

78. A compound of formula (II) or a pharmaceutically acceptable salt or solvate thereof:

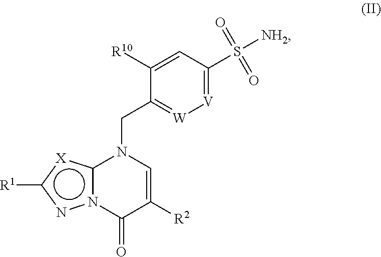

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   V is N or CR6;
   W is N or CR7;
   each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
   R10 is H or halo; and
   R11 is H or C1-C3 alkyl.

78a. The compound of Clause 78, wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered cycloalkyl or heterocycloalkyl having one or more (e.g., 1 or 2) ring heteroatoms independently selected from N, O, and S;
R6 is selected from H, halo, hydroxy, C1-C4 alkyl, C1-C4 alkoxy, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo; and
R7 is selected from H, C1-C4 alkyl, hydroxy, —N(H)(R11), and halo.

79. The compound of Clause 78 or Clause 78a, wherein X is N.

80. The compound of Clause 78 or Clause 78a, wherein X is C(CH₂)ₘR5, and R5 is as defined in Clause 78 or Clause 78a.

81. The compound of any of Clauses 78, 78a or 80, wherein m is 0.

82. The compound of any of Clauses 78, 78a or 80, wherein m is 1.

83. The compound of any of Clauses 78, 78a, 80 or 81, wherein X is CH.

84. The compound of any of Clauses 78, 78a, 80 or 81, wherein X is C-halo, optionally wherein halo is Cl or F (e.g. F).

85. The compound of any of Clauses 78, 78a, 80 or 81, wherein X is CR5, and R5 is OMe.

86. The compound of any of Clauses 78, 78a, 80 or 81, wherein X is CR5, and R5 is CN.

87. The compound of any of Clauses 78, 78a, 80 or 81, wherein X is CR5, and R5 is Me.

88. The compound of any of Clauses 78, 78a, 80, 81 or 83, wherein X is CR5, and R5 is 2H.

88a. The compound of any of Clauses 78, 78a, 80, 81 or 83, wherein X is CR5, and R5 is 1H.

89. The compound of any of Clauses 78 to 88a, wherein R1 is H or halo.

90. The compound of any of Clauses 78 to 88a, wherein R1 is C1-C6 alkyl or C1-C6 haloalkyl.

90a. The compound of any of Clauses 78 to 88a or 90, wherein R1 is C1-C6 alkyl.

91. The compound of any of Clauses 78 to 88a, wherein R1 is C3-C6 cycloalkyl (e.g. cyclopropyl), optionally substituted with one or more (e.g., 1, 2, 3, or 4) substituents independently selected from C1-C3 alkyl, C3-C6 cycloalkyl and halo.

91a. The compound of any of Clauses 78 to 88a or 91, wherein R1 is C3-C6 cycloalkyl.

91b. The compound of any of Clauses 78 to 88a, 91 or 91a, wherein R1 is cyclopropyl.

91c. The compound of any of Clauses 78 to 88a, wherein R1 is cyclopropyl or ethyl.

92. The compound of any of Clauses 78 to 88a, wherein R1 is C1-C6 alkoxyl or C1-C6 haloalkoxyl.

92a. The compound of any of Clauses 78 to 88a or 92, wherein R1 is C1-C6 alkoxyl.

92b. The compound of any of Clauses 78 to 89, wherein R1 is H.

93. The compound of Clause 78 or Clause 78a, wherein R1 is H or is selected from:

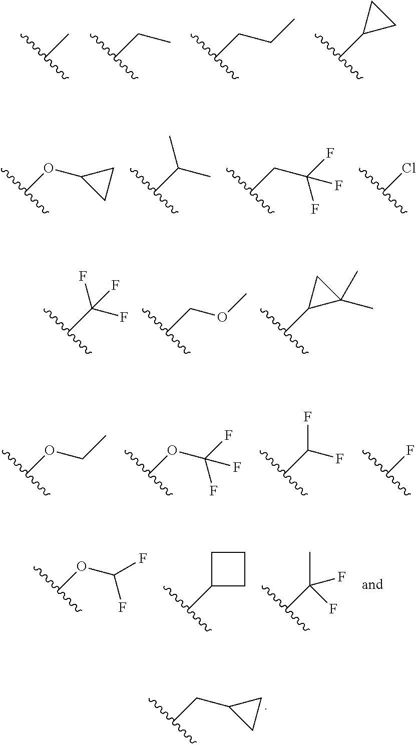

94. The compound of any of Clauses 78, 78a or 93, wherein R1 is H or is selected from:

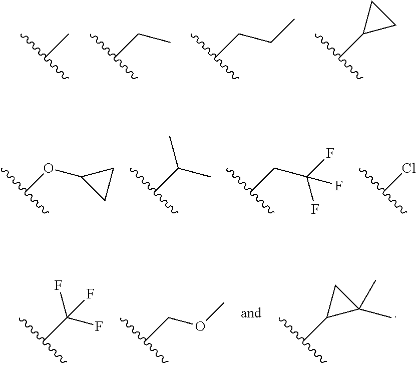

95. The compound of any of Clauses 78 or 80 to 82, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

96. The compound of any of Clauses 78, 78a, 80 to 82 or 95, wherein R5 and R1 taken together with the atoms to which they are attached form a 5-6 membered cycloalkyl.

97. The compound of any of Clauses 78, 78a, 80 to 82 or 95, wherein R5 and R1 taken together with the atoms to which they are attached form a 5-6 membered heterocycloalkyl having one or more (e.g., 1 or 2) ring heteroatoms independently selected from N, O, and S.

98. The compound of any of Clauses 78, 80 to 82 or 95, wherein R5 and R1 taken together with the atoms to which they are attached form a phenyl, optionally substituted with one or more (e.g., 1, 2, 3, or 4) substituents independently selected from C1-C6 alkoxyl.

99. The compound of any of Clauses 78, 80 to 82 or 95, wherein R5 and R1 taken together with the atoms to which they are attached form a 5-6 membered heteroaryl having one or more (e.g., 1 or 2) ring heteroatoms independently selected from N, O, and S.

100. The compound of any of Clauses 78, 80 to 82 or 95, wherein the compound is selected from any one of the following structures:

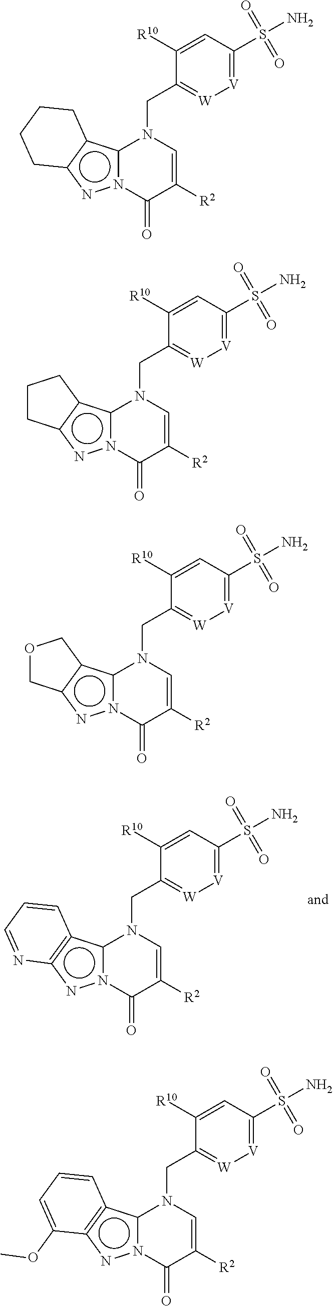

wherein R2, V, W and R10 are as defined in Clause 78 or Clause 78a.

101. The compound of any of Clauses 78 to 100, wherein R2 is C1-C6 alkyl or C1-C6 haloalkyl.

101a. The compound of any of Clauses 78 to 101, wherein R2 is C1-C6 alkyl.

101b. The compound of any of Clauses 78 to 101a, wherein R2 is C1-C3 alkyl.

101c. The compound of any of Clauses 78 to 101b, wherein R2 is isopropyl.

102. The compound of any of Clauses 78 to 100, wherein R2 is C3-C6 cycloalkyl, optionally substituted with one or more (e.g., 1, 2, 3, or 4) halo.

103. The compound of any of Clauses 78 to 100, wherein R2 is phenyl, optionally substituted with one or more (e.g., 1, 2, 3, or 4) halo.

104. The compound of any of Clauses 78 to 100, wherein R2 is 6-membered heteroaryl having one or more (e.g. 1, 2, 3, or 4) ring heteroatoms independently selected from N, O, and S, wherein the heteroaryl is optionally substituted with one or more (e.g., 1, 2, 3, or 4) halo.

105. The compound of any of Clauses 78 to 100, wherein R2 is selected from:

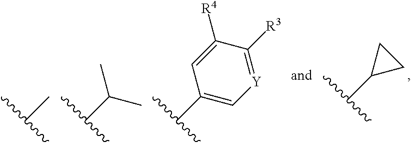

wherein Y is CH or N, R3 is H or halo and R4 is H or halo (optionally halo is F).

106. The compound of any of Clauses 78 to 100 or 105, wherein R2 is selected from:

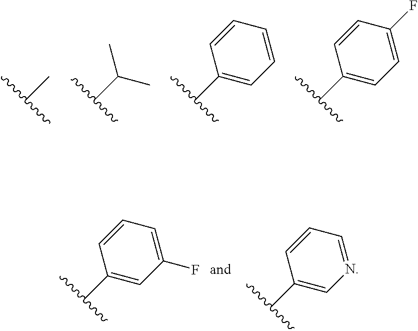

107. The compound of any of Clauses 78 to 106, wherein V is N.

108. The compound of any of Clauses 78 to 106, wherein V is CR6, and R6 is selected from H, C1-C4 alkyl, C1-C4 alkoxy, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo.

108a. The compound of any of Clauses 78 to 106, wherein V is CR6, and R6 is selected from H, halo, hydroxy, C1-C4 alkyl, C1-C4 haloalkyl, C1-C4 alkoxy, C1-C4 haloalkoxy, C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo.

109. The compound of any of Clauses 78 to 108a, wherein W is N.

110. The compound of any of Clauses 78 to 108a, wherein W is CR7, R7 is selected from H, C1-C4 alkyl, hydroxy, —N(H)(R11), and halo, and R11 is as defined in Clause 78.

111. The compound of any of Clauses 78 to 110, wherein R10 is H.

112. The compound of any of Clauses 78 to 110, wherein R10 is halo.

112a. The compound of any of Clauses 78 to 106, wherein R10 is H, and V and W are independently selected from CH, C-C1-C3 alkyl and C-halo.

112b. The compound of any of Clauses 78 to 106 or 112a, wherein R10 is H, and V and W are independently selected from CH, CMe and C-halo (e.g. CF).

112c. The compound of any of Clauses 78 to 106, 112a or 112b, wherein R10 is H, and V and W are both CH.

113. The compound of any of Clauses 78 to 112, wherein R11 is H.

114. The compound of any of Clauses 78 to 112, wherein R11 is C1-C3 alkyl (e.g. Me).

115. The compound of Clause 78, wherein the compound is selected from:

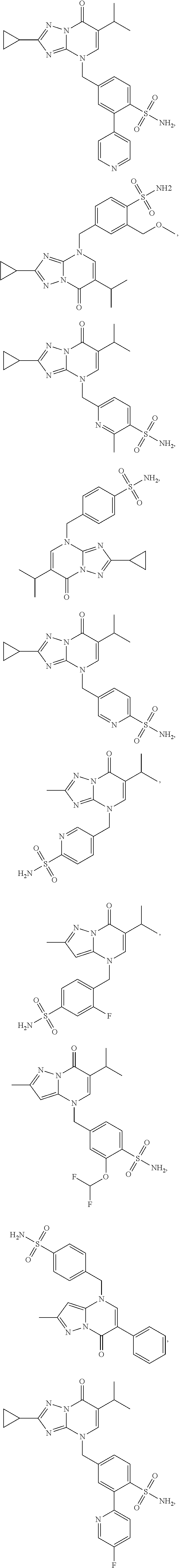

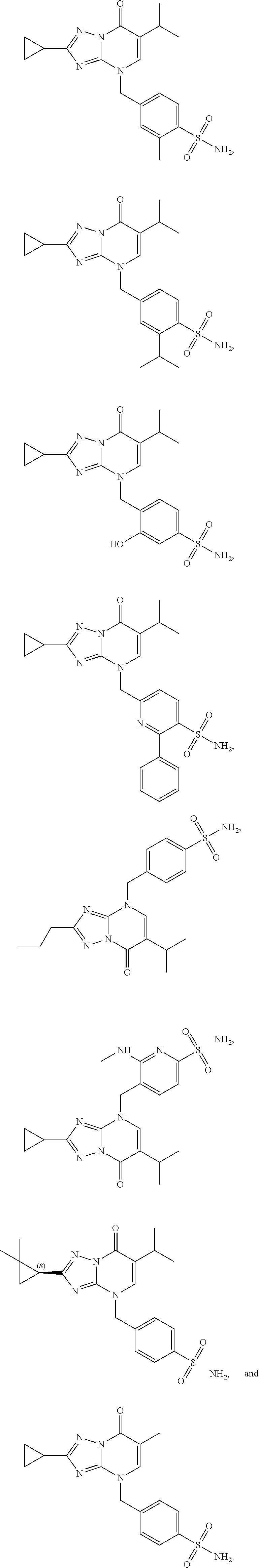

116. A compound of formula (III) or a pharmaceutically acceptable salt or solvate thereof:

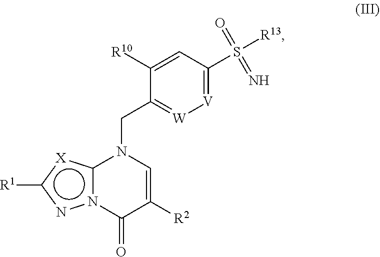

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   n is 1, 2, or 3;
   m is 0 or 1;
   V is N or CR6;
   W is N or CR7;
   each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
   R10 is H or halo;
   R11 is H or C1-C3 alkyl; and
   R13 is C1-C3 alkyl, C1-C3 haloalkyl, or C3-C6 cycloalkyl, wherein cycloalkyl is optionally substituted with one or more (e.g., 1, 2, 3, or 4) halo; or
   R13 and R6 together form a 5-6 membered heterocycloalkyl optionally having one or more (e.g., 1, 2 or 3) additional ring heteroatoms independently selected from N, S and O.

116a. The compound of Clause 116, wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered cycloalkyl or heterocycloalkyl having one or more (e.g., 1 or 2) ring heteroatoms independently selected from N, O, and S;
R6 is selected from H, halo, hydroxy, C1-C4 alkyl, C1-C4 alkoxy, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R7 is selected from H, C1-C4 alkyl, hydroxy, —N(H)(R11), and halo; and
R13 is C1-C3 alkyl, C1-C3 haloalkyl, or C3-C6 cycloalkyl, wherein cycloalkyl is optionally substituted with one or more (e.g., 1, 2, 3, or 4) halo.

117. The compound of Clause 116 or Clause 116a, wherein X is N.

118. The compound of Clause 116 or Clause 116a, wherein X is C(CH₂)ₘR5, and R5 is as defined in Clause 116 or Clause 116a.

119. The compound of any of Clauses 116, 116a or 118, wherein m is 0.

120. The compound of any of Clauses 116, 116a or 118, wherein m is 1.

121. The compound of any of Clauses 116, 116a, 118 or 119, wherein X is CH.

122. The compound of any of Clauses 116, 116a, 118 or 119, wherein X is C-halo, optionally wherein halo is Cl or F (e.g. F).

123. The compound of any of Clauses 116, 116a, 118 or 119, wherein X is CR5, and R5 is OMe.

124. The compound of any of Clauses 116, 116a, 118 or 119, wherein X is CR5, and R5 is CN.

125. The compound of any of Clauses 116, 116a, 118 or 119, wherein X is CR5, and R5 is Me.

126. The compound of any of Clauses 116, 116a, 118, 119 or 121, wherein X is CR5, and R5 is 2H.

126a. The compound of any of Clauses 116, 116a, 118, 119 or 121, wherein X is CR5, and R5 is 1H.

127. The compound of any of Clauses 116 to 126a, wherein R1 is H or halo.

128. The compound of any of Clauses 116 to 126a, wherein R1 is C1-C6 alkyl or C1-C6 haloalkyl.

128a. The compound of any of Clauses 116 to 126a or 128, wherein R1 is C1-C6 alkyl.

129. The compound of any of Clauses 116 to 126a, wherein R1 is C3-C6 cycloalkyl (e.g. cyclopropyl), optionally substituted with one or more (e.g., 1, 2, 3, or 4) substituents independently selected from C1-C3 alkyl, C3-C6 cycloalkyl and halo.

129a. The compound of any of Clauses 116 to 126a or 129, wherein R1 is C3-C6 cycloalkyl.

129b. The compound of any of Clauses 116 to 126a, 129 or 129a, wherein R1 is cyclopropyl.

129c. The compound of any of Clauses 116 to 126a, wherein R1 is cyclopropyl or ethyl.

130. The compound of any of Clauses 116 to 126a, wherein R1 is C1-C6 alkoxyl or C1-C6 haloalkoxyl.

130a. The compound of any of Clauses 116 to 126a or 130, wherein R1 is C1-C6 alkoxyl.

130b. The compound of any of Clauses 116 to 127, wherein R1 is H.

131. The compound of Clause 116 or Clause 116a, wherein R1 is H or is selected from:

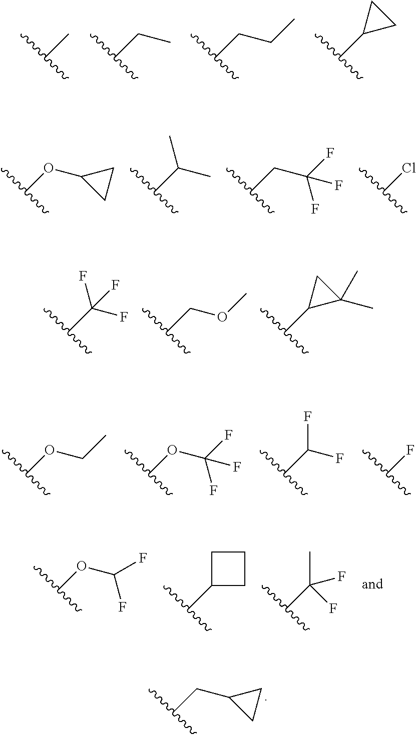

132. The compound of any of Clauses 116, 116a or 131, wherein R1 is H or is selected from:

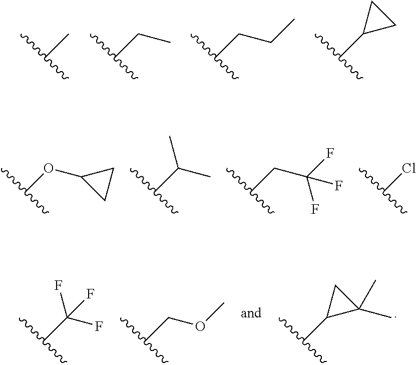

133. The compound of any of Clauses 116 or 118 to 120, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

134. The compound of any of Clauses 116, 116a, 118 to 120 or 133, wherein R5 and R1 taken together with the atoms to which they are attached form a 5-6 membered cycloalkyl.

135. The compound of any of Clauses 116, 116a, 118 to 120 or 133, wherein R5 and R1 taken together with the atoms to which they are attached form a 5-6 membered heterocycloalkyl having one or more (e.g., 1 or 2) ring heteroatoms independently selected from N, O, and S.

136. The compound of any of Clauses 116, 118 to 120 or 133, wherein R5 and R1 taken together with the atoms to which they are attached form a phenyl, optionally substituted with one or more (e.g., 1, 2, 3, or 4) substituents independently selected from C1-C6 alkoxyl.

137. The compound of any of Clauses 116, 118 to 120 or 133, wherein R5 and R1 taken together with the atoms to which they are attached form a 5-6 membered heteroaryl having one or more (e.g., 1 or 2) ring heteroatoms independently selected from N, O, and S.

138. The compound of any of Clauses 116, 118 to 120 or 133, wherein the compound is selected from any one of the following structures:

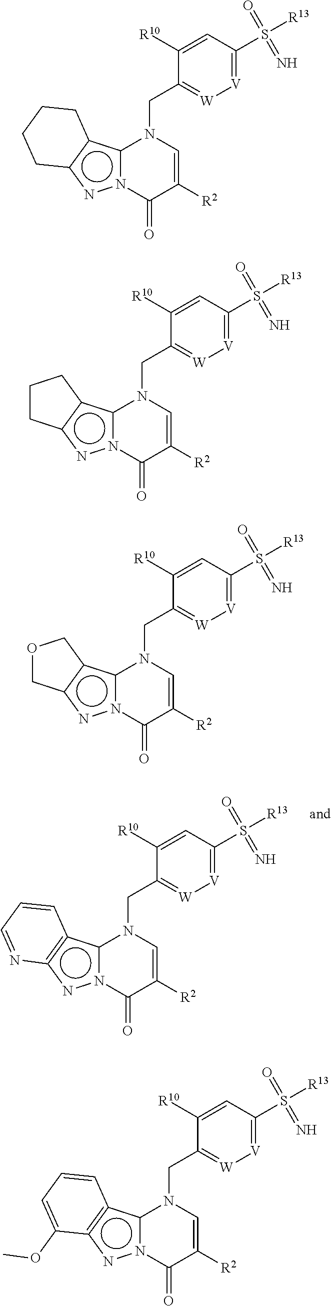

wherein R2, V, W, R10 and R13 are as defined in Clause 116 or Clause 116a.

139. The compound of any of Clauses 116 to 138, wherein R2 is C1-C6 alkyl or C1-C6 haloalkyl. 139a. The compound of any of Clauses 116 to 139, wherein R2 is C1-C6 alkyl.

139b. The compound of any of Clauses 116 to 139a, wherein R2 is C1-C3 alkyl.

139c. The compound of any of Clauses 116 to 139b, wherein R2 is isopropyl.

140. The compound of any of Clauses 116 to 138, wherein R2 is C3-C6 cycloalkyl, optionally substituted with one or more (e.g., 1, 2, 3, or 4) halo.

141. The compound of any of Clauses 116 to 138, wherein R2 is phenyl, optionally substituted with one or more (e.g., 1, 2, 3, or 4) halo.

142. The compound of any of Clauses 116 to 138, wherein R2 is 6-membered heteroaryl having one or more (e.g. 1, 2, 3, or 4) ring heteroatoms independently selected from N, O, and S, wherein the heteroaryl is optionally substituted with one or more (e.g., 1, 2, 3, or 4) halo.

143. The compound of any of Clauses 116 to 138, wherein R2 is selected from:

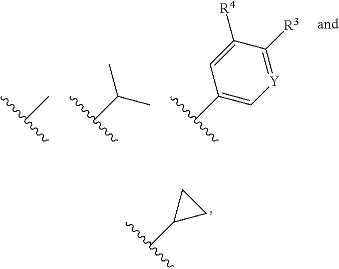

wherein Y is CH or N, R3 is H or halo and R4 is H or halo (optionally halo is F).

144. The compound of any of Clauses 116 to 138 or 143, wherein R2 is selected from:

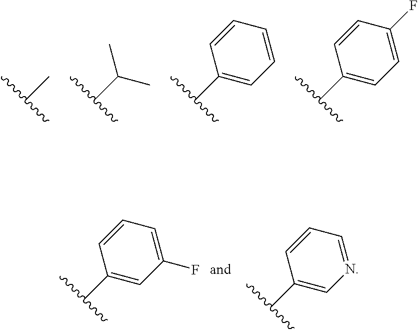

145. The compound of any of Clauses 116 to 144, wherein V is N.

146. The compound of any of Clauses 116 to 144, wherein V is CR6, and R6 is selected from H, C1-C4 alkyl, C1-C4 alkoxy, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo.

146a. The compound of any of Clauses 116 to 144, wherein V is CR6, and R6 is selected from H, C1-C4 alkyl, C1-C4 haloalkyl, C1-C4 alkoxy, C1-C4 haloalkoxy, C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo.

147. The compound of any of Clauses 116 to 146a, wherein W is N.

148. The compound of any of Clauses 116 to 146a, wherein W is CR7, R7 is selected from H, C1-C4 alkyl, hydroxy, —N(H)(R11), and halo, and R11 is as defined in Clause 116.

149. The compound of any of Clauses 116 to 148, wherein R10 is H.

150. The compound of any of Clauses 116 to 148, wherein R10 is halo.

150a. The compound of any of Clauses 116 to 144, wherein R10 is H, and V and W are independently selected from CH, C-C1-C3 alkyl and C-halo.

150b. The compound of any of Clauses 116 to 144 or 150a, wherein R10 is H, and V and W are independently selected from CH, CMe and C-halo (e.g. CF).

150c. The compound of any of Clauses 116 to 144, 150a or 150b, wherein R10 is H, and V and W are both CH.

151. The compound of any of Clauses 116 to 150, wherein R11 is H.

152. The compound of any of Clauses 116 to 150, wherein R11 is C1-C3 alkyl (e.g. Me).

153. The compound of any of Clauses 116 to 152, wherein R13 is C1-C3 alkyl (e.g. C₁H³ or C²H₃) or C1-C3 haloalkyl (e.g. CF₃ or CHF₂).

154. The compound of any of Clauses 116 to 153, wherein R13 is Me (e.g. C₁H³ or C²H₃).

155. The compound of any of Clauses 116 to 152, wherein R13 is C3-C6 cycloalkyl (e.g. cyclopropyl), optionally substituted with one or more (e.g., 1, 2, 3, or 4) halo.

156. The compound of any of Clauses 116 to 152 or 155, wherein R13 is C3-C6 cycloalkyl.

156a. The compound of any of Clauses 116 to 152, 155 or 156, wherein R13 is cyclopropyl.

156b. The compound of any of Clauses 116 to 152, wherein R13 is selected from C3-C6 cycloalkyl and C1-C3 alkyl.

156c. The compound of any of Clauses 116 to 152 or 156b, wherein R13 is selected from cyclopropyl, methyl (e.g. C₁H³ or C²H₃), ethyl and isopropyl.

157. The compound of any of Clauses 116 or 117 to 152, wherein R13 and R6 together form a 5-6 membered heterocyclomikyi optionally having one or more (e.g., 1, 2 or 3) additional ring heteroatoms Independently selected from N, S and O.

158. The compound of any of Clauses 116 or 117 to 152, wherein the compound is selected from a compound of the formula:

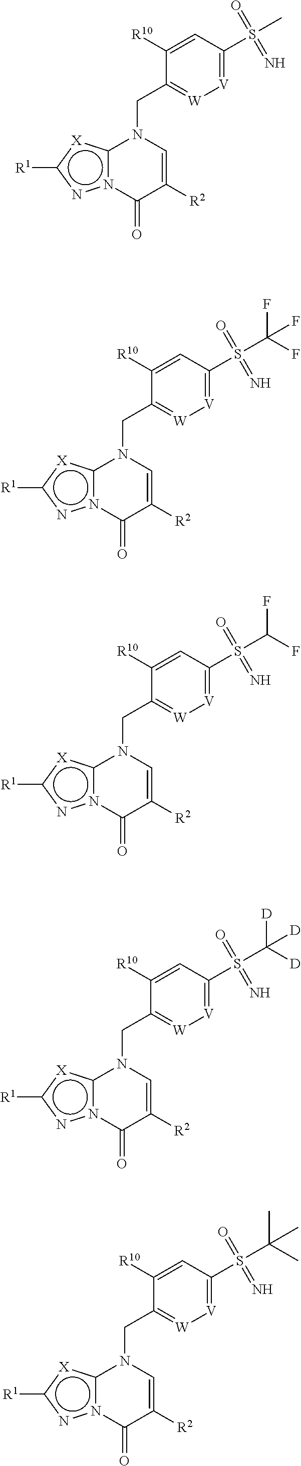

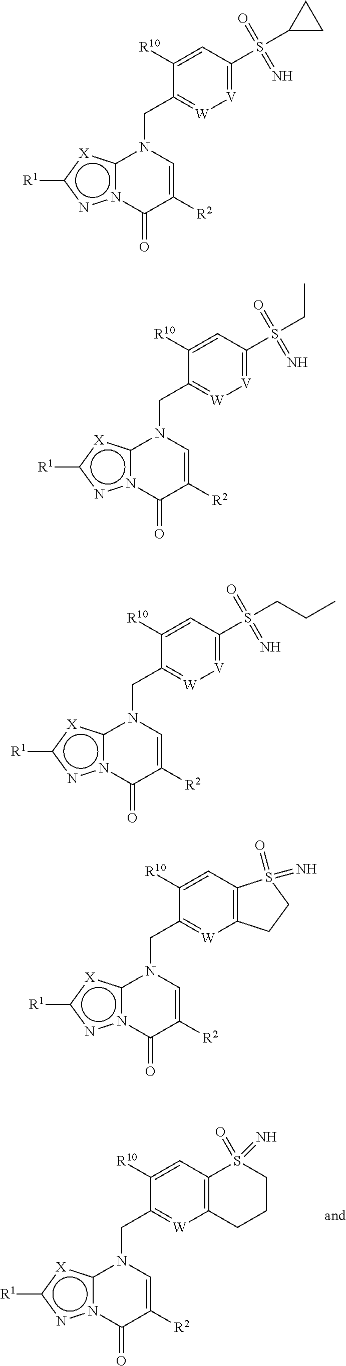

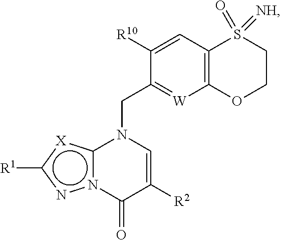

wherein X, R1, R2, V, W and R10 are as defined in Clause 116 or Clause 116a.

159. The compound of any of Clauses 116 to 152 or 158, wherein the compound is selected from any one of the following structures:

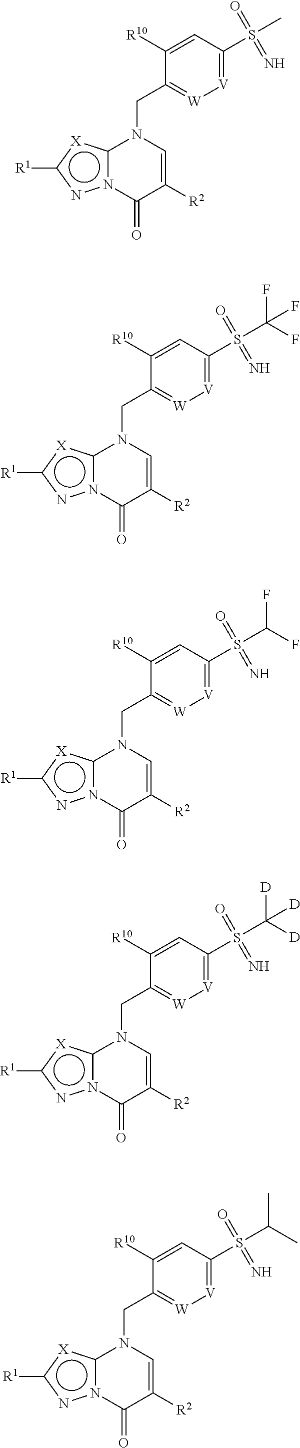

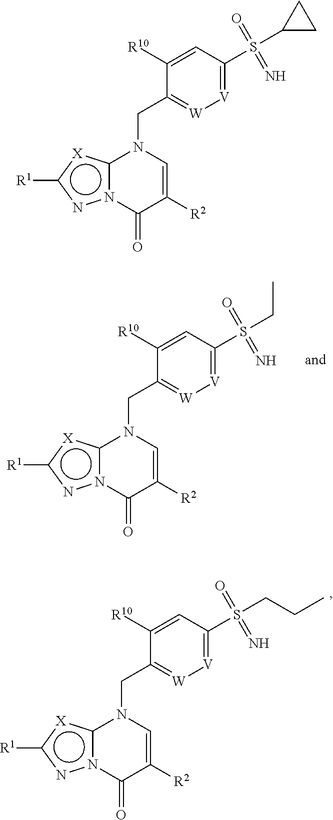

wherein R1, X, R2, R10, V, and W are as defined in Clause 116 or Clause 116a.

159a. The compound of any of Clauses 116 to 152, 158 or 159, wherein the compound is selected from any one of the following structures:

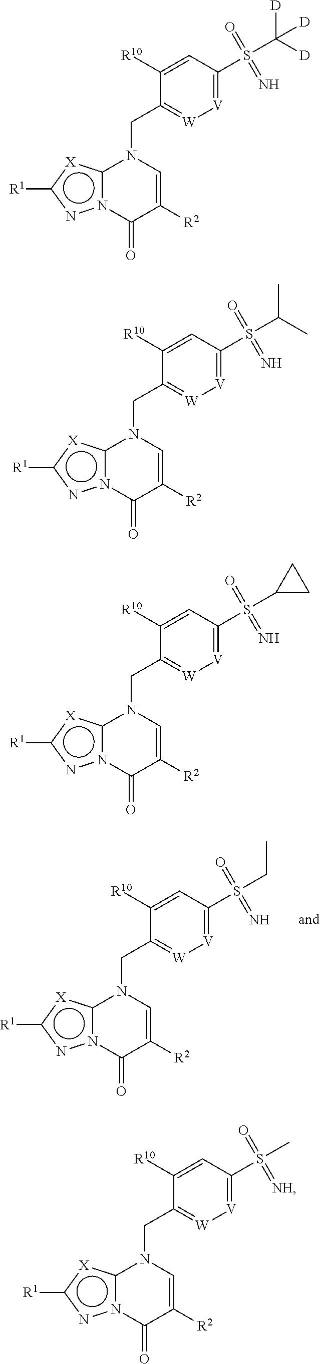

wherein R1, X, R2, R10, V, and W are as defined in Clause 116 or Clause 116a.

160. The compound of any of Clauses 116, 117 to 152 or 158, wherein the compound is selected from any one of the following structures:

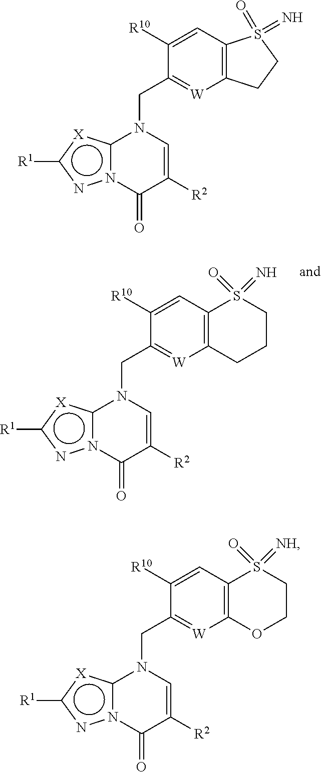

wherein R1, R2, X, R10 and W are as defined in Clause 116 or Clause 116a.

161. The compound of any of Clauses 116 to 154, 156b, 156c or 158 to 159a, wherein the compound is

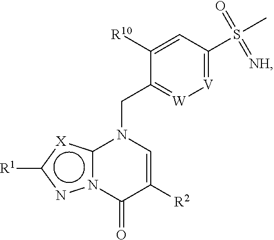

wherein R1, R2, X, R10, V and W are as defined in Clause 116 or Clause 116a.

162. The compound of Clause 116, wherein the compound is selected from a compound of any of the following structures:

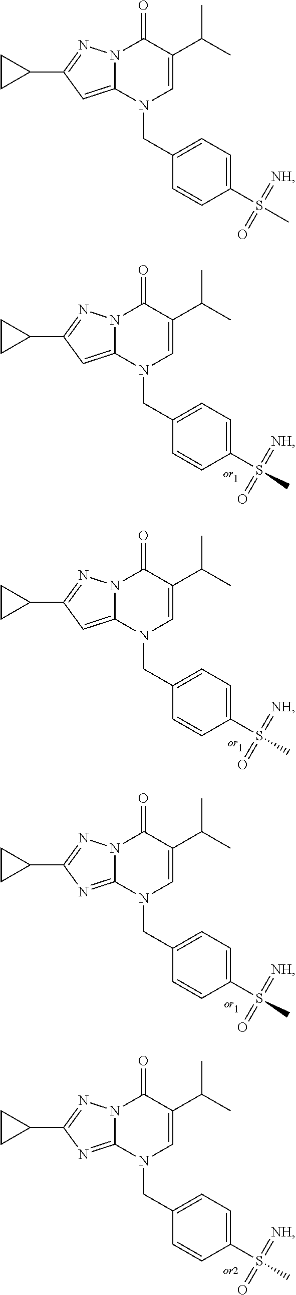 [SMILES: CC(C)c1cn(Cc2ccc(S(C)(=N)=O)cc2)c2cc(C3CC3)nn2c1=O ; CC(C)c1cn(Cc2ccc([S@](C)(=N)=O)cc2)c2cc(C3CC3)nn2c1=O ; CC(C)c1cn(Cc2ccc([S@@](C)(=N)=O)cc2)c2cc(C3CC3)nn2c1=O ; CC(C)c1cn(Cc2ccc([S@](C)(=N)=O)cc2)c2nc(C3CC3)nn2c1=O ; CC(C)c1cn(Cc2ccc([S@@](C)(=N)=O)cc2)c2nc(C3CC3)nn2c1=O]

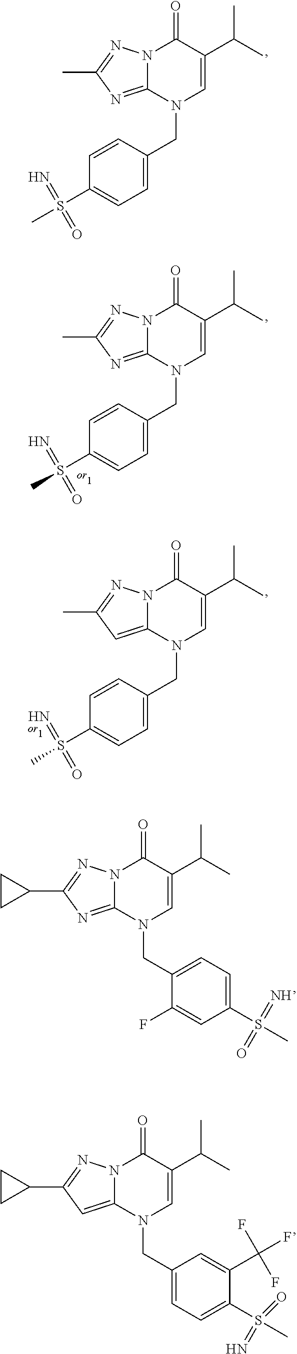

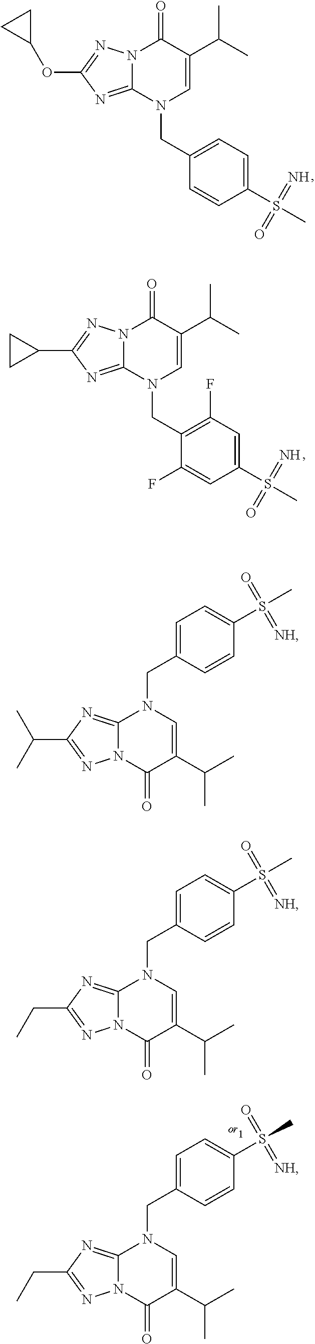

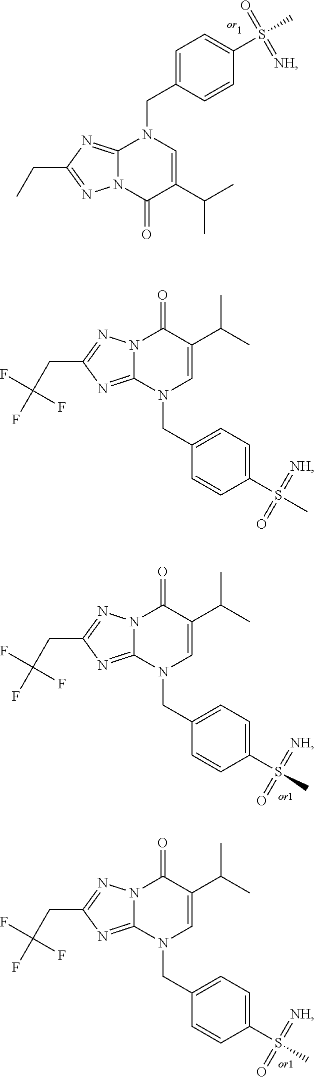 [SMILES: CCc1nc2n(Cc3ccc([S@](C)(=N)=O)cc3)cc(C(C)C)c(=O)n2n1 ; CC(C)c1cn(Cc2ccc(S(C)(=N)=O)cc2)c2nc(CC(F)(F)F)nn2c1=O ; CC(C)c1cn(Cc2ccc([S@](C)(=N)=O)cc2)c2nc(CC(F)(F)F)nn2c1=O ; CC(C)c1cn(Cc2ccc([S@@](C)(=N)=O)cc2)c2nc(CC(F)(F)F)nn2c1=O]

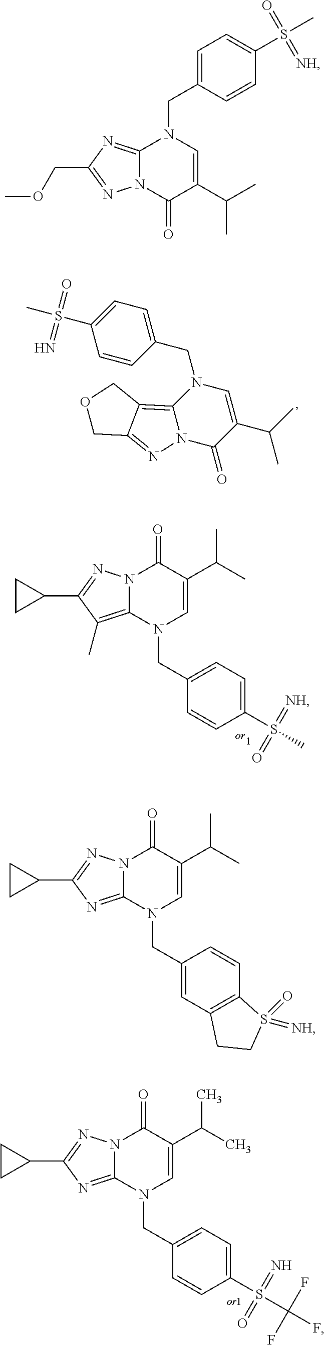

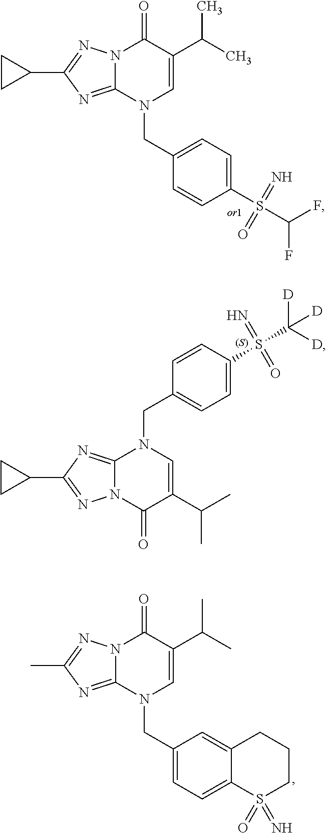

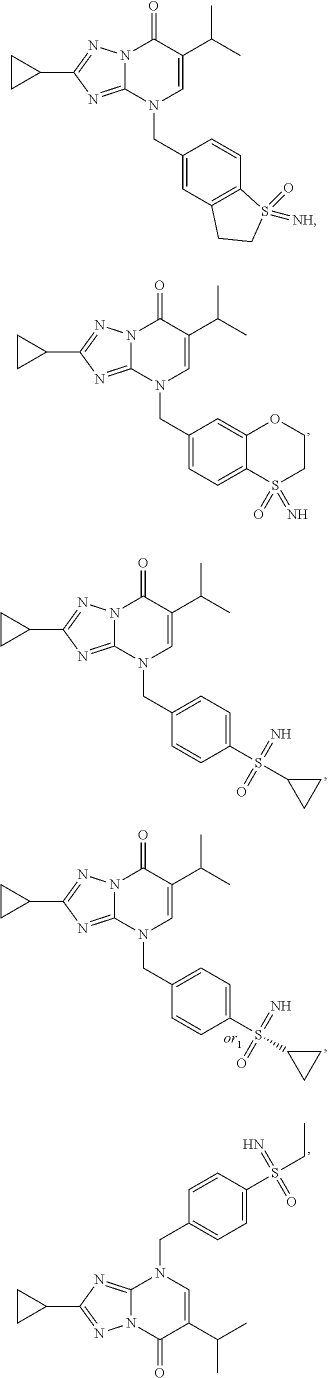

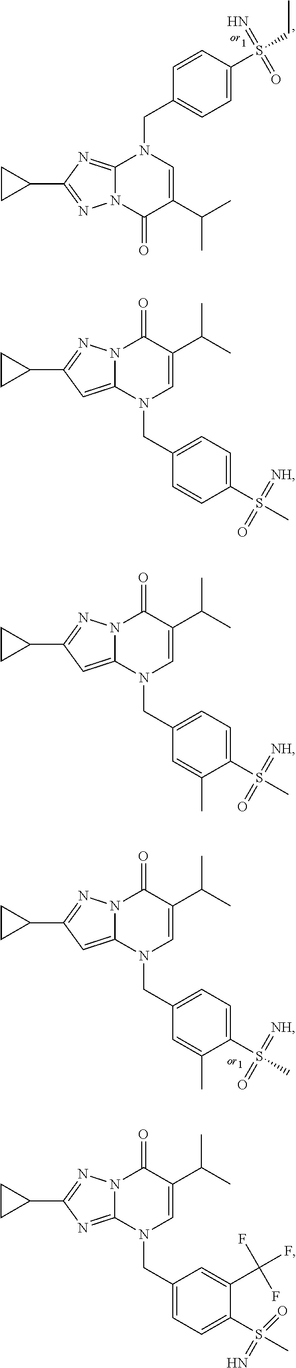

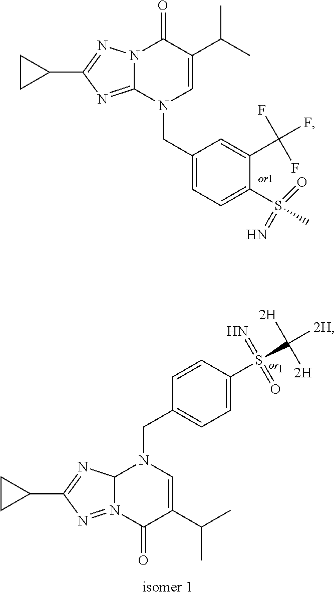

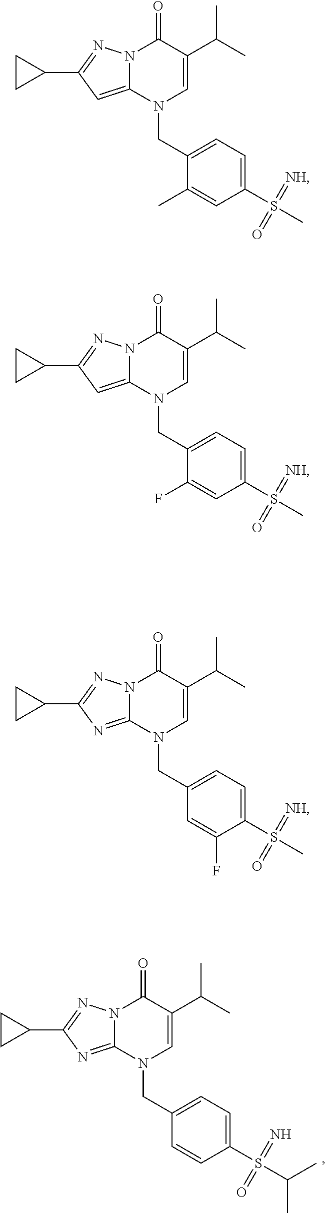 [SMILES: Cc1cc(S(C)(=N)=O)ccc1Cn1cc(C(C)C)c(=O)n2nc(C3CC3)cc12 ; CC(C)c1cn(Cc2ccc(S(C)(=N)=O)cc2F)c2cc(C3CC3)nn2c1=O ; CC(C)c1cn(Cc2ccc(S(C)(=N)=O)c(F)c2)c2nc(C3CC3)nn2c1=O ; CC(C)c1cn(Cc2ccc(S(=N)(=O)C(C)C)cc2)c2nc(C3CC3)nn2c1=O]

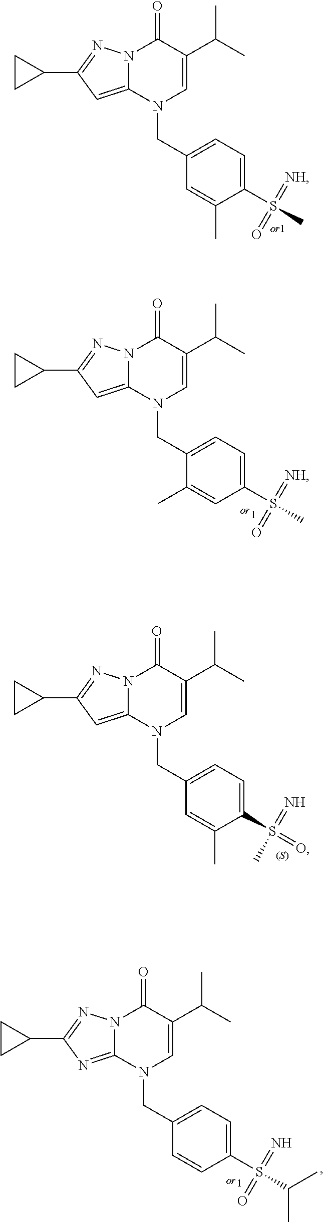

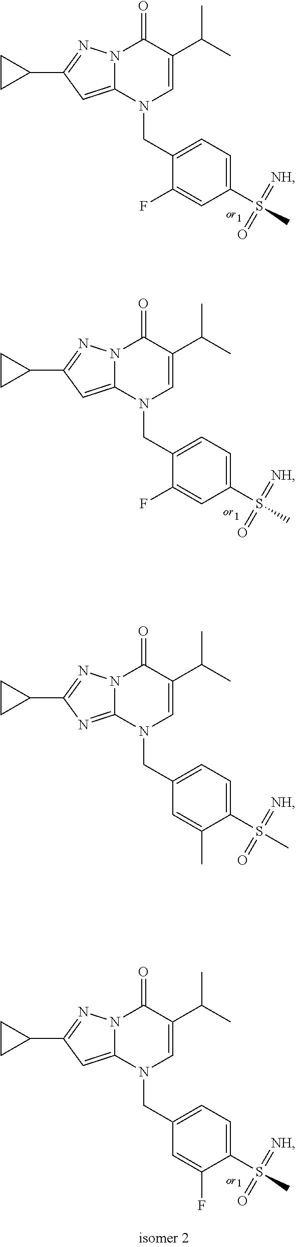 [SMILES: CC(C)c1cn(Cc2ccc([S@](C)(=N)=O)cc2F)c2cc(C3CC3)nn2c1=O ; CC(C)c1cn(Cc2ccc([S@@](C)(=N)=O)cc2F)c2cc(C3CC3)nn2c1=O ; Cc1cc(Cn2cc(C(C)C)c(=O)n3nc(C4CC4)nc23)ccc1S(C)(=N)=O ; CC(C)c1cn(Cc2ccc([S@](C)(=N)=O)c(F)c2)c2cc(C3CC3)nn2c1=O]

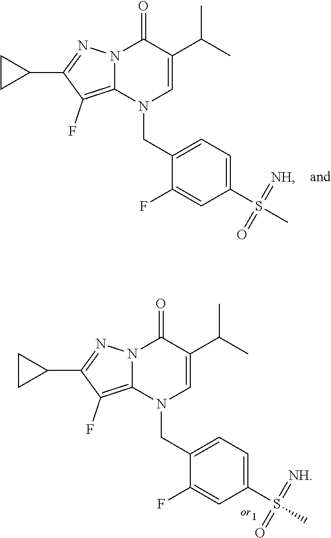 [SMILES: CC(C)c1cn(Cc2ccc(S(C)(=N)=O)cc2F)c2c(F)c(C3CC3)nn2c1=O ; CC(C)c1cn(Cc2ccc([S@@](C)(=N)=O)cc2F)c2c(F)c(C3CC3)nn2c1=O]

163. The compound of Clause 116 or Clause 162, wherein the compound is selected from a compound of any of the following structures:

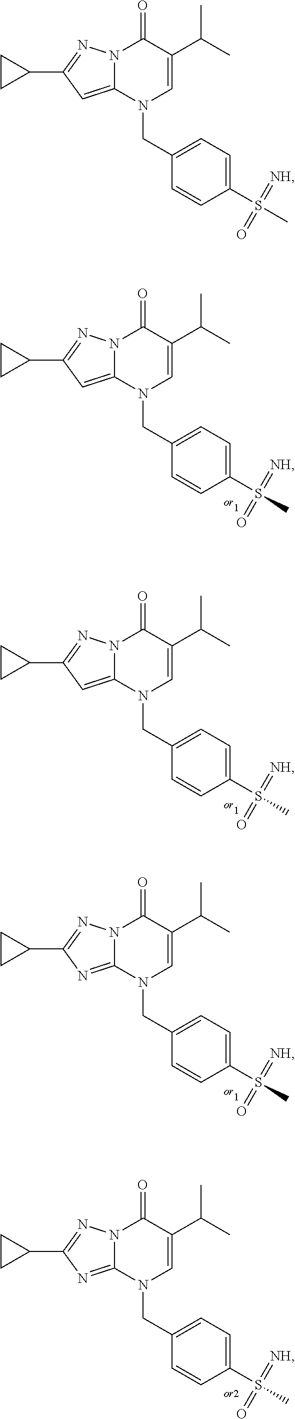 [SMILES: CC(C)c1cn(Cc2ccc(S(C)(=N)=O)cc2)c2cc(C3CC3)nn2c1=O ; CC(C)c1cn(Cc2ccc([S@](C)(=N)=O)cc2)c2cc(C3CC3)nn2c1=O ; CC(C)c1cn(Cc2ccc([S@@](C)(=N)=O)cc2)c2cc(C3CC3)nn2c1=O ; CC(C)c1cn(Cc2ccc([S@](C)(=N)=O)cc2)c2nc(C3CC3)nn2c1=O ; CC(C)c1cn(Cc2ccc([S@@](C)(=N)=O)cc2)c2nc(C3CC3)nn2c1=O]

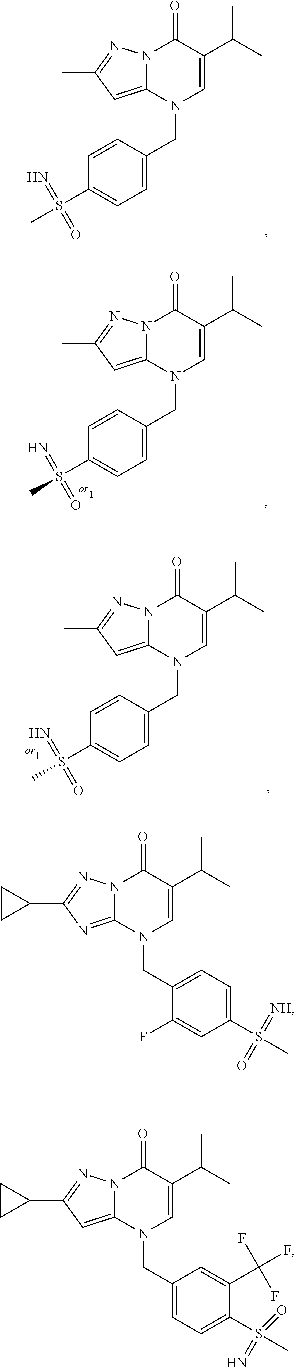

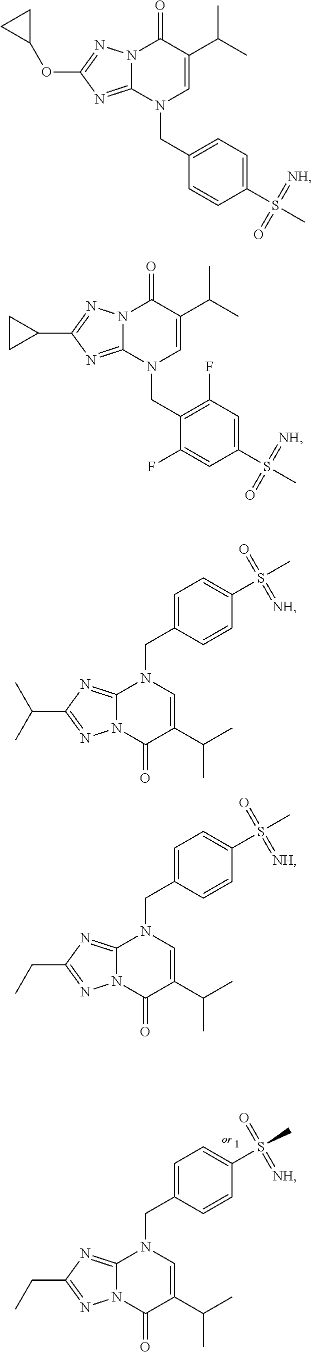

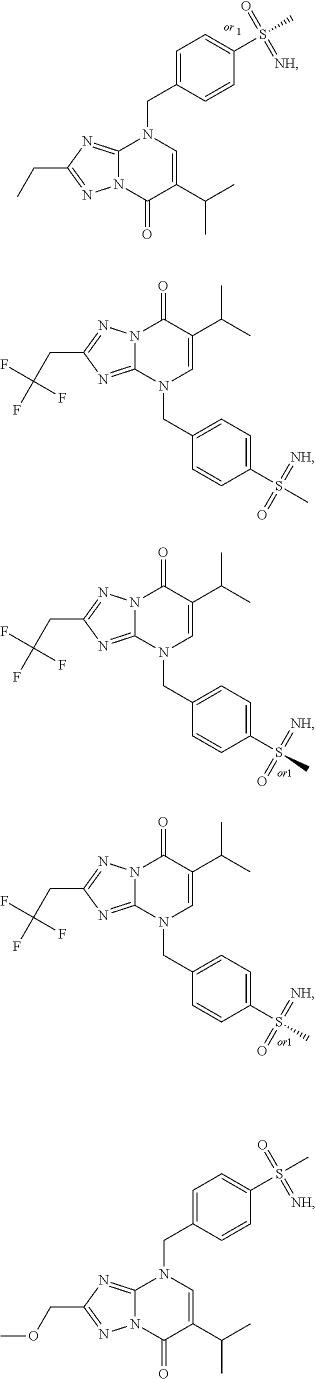

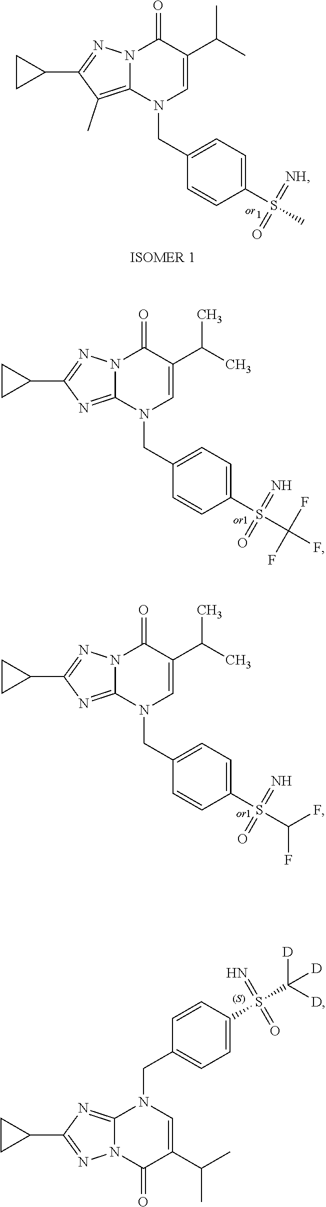

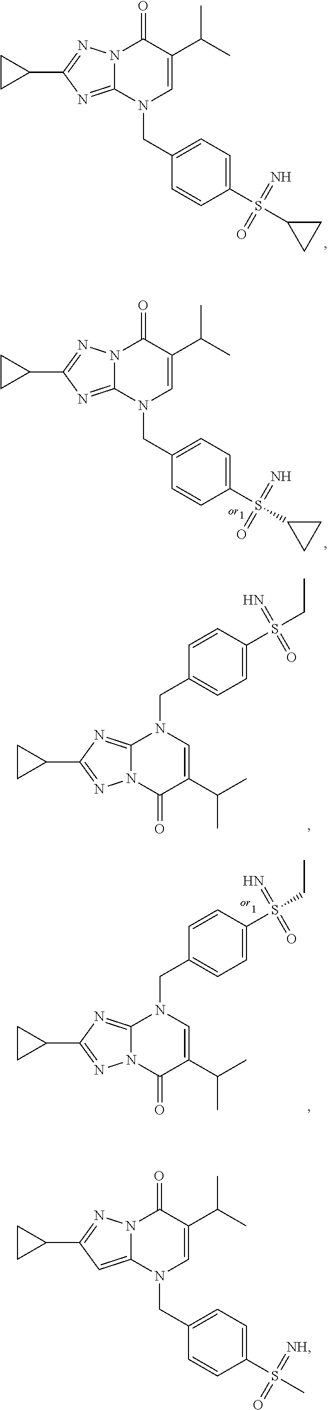

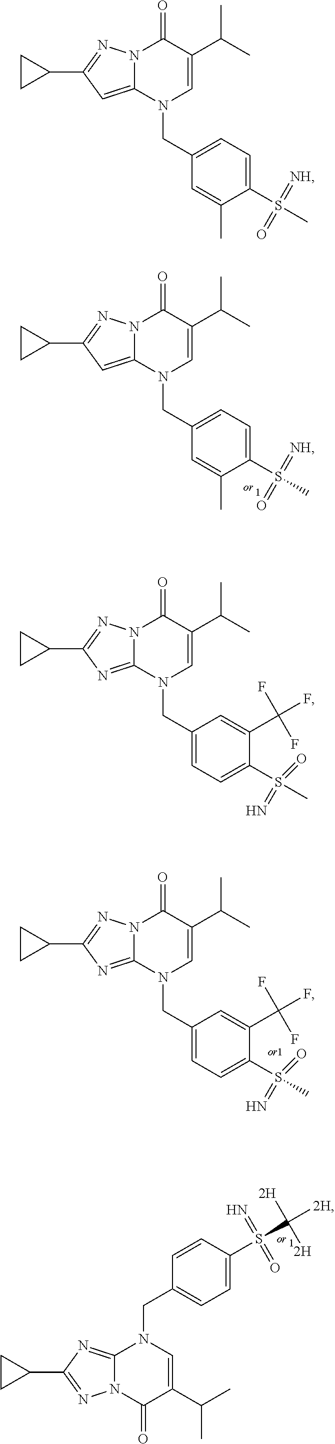

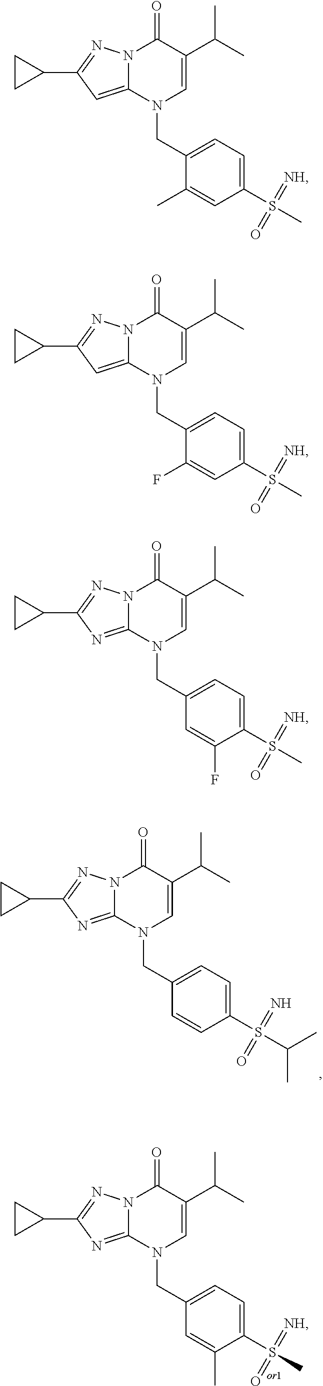 [SMILES: Cc1cc(S(C)(=N)=O)ccc1Cn1cc(C(C)C)c(=O)n2nc(C3CC3)cc12 ; CC(C)c1cn(Cc2ccc(S(C)(=N)=O)cc2F)c2cc(C3CC3)nn2c1=O ; CC(C)c1cn(Cc2ccc(S(C)(=N)=O)c(F)c2)c2nc(C3CC3)nn2c1=O ; CC(C)c1cn(Cc2ccc(S(=N)(=O)C(C)C)cc2)c2nc(C3CC3)nn2c1=O ; Cc1cc(Cn2cc(C(C)C)c(=O)n3nc(C4CC4)nc23)ccc1[S@](C)(=N)=O]

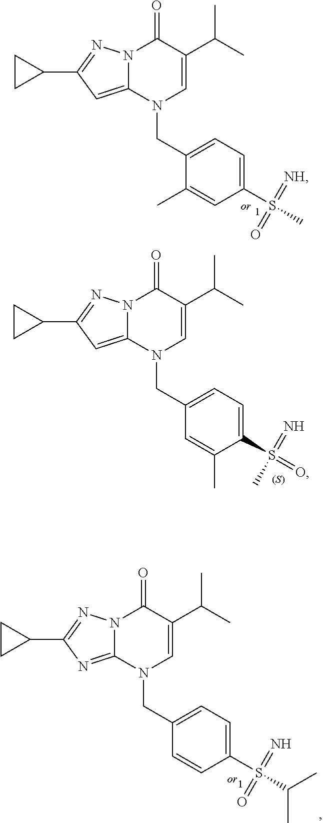

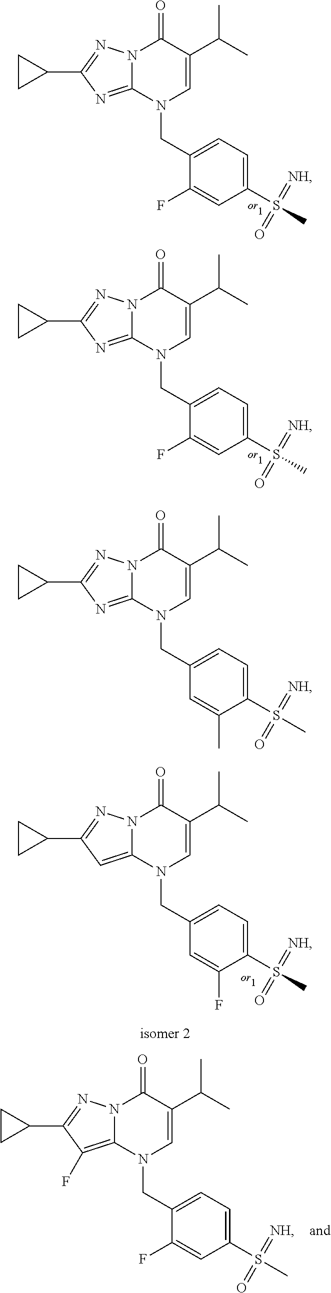

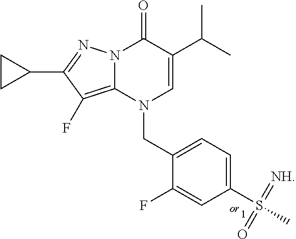 [SMILES: CC(C)c1cn(Cc2ccc([S@@](C)(=N)=O)cc2F)c2c(F)c(C3CC3)nn2c1=O]

164. A compound of formula (IV) or (V) or a pharmaceutically acceptable salt or solvate thereof:

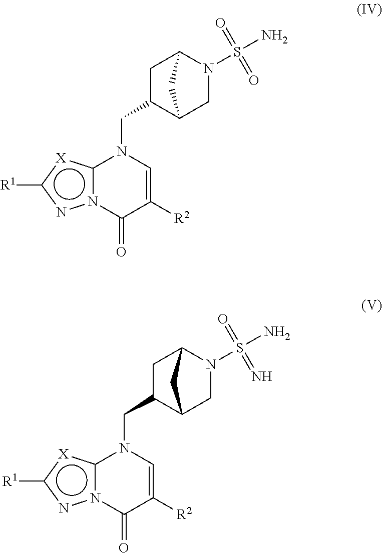

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo; and
   m is 0 or 1.

164a. The compound of Clause 164, wherein:
X is N or C(CH₂)ₘR5;
R5 is selected from H, halo, Me, CN, and OMe; and
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered cycloalkyl or heterocycloalkyl having one or more (e.g., 1 or 2) ring heteroatoms independently selected from N, O, and S.

165. The compound of Clause 164 or Clause 164a, wherein the compound has formula (IV):

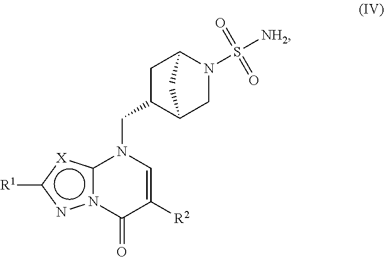

wherein R1, R2 and X are as defined in Clause 164 or Clause 164a.

166. The compound of Clause 164 or Clause 164a, wherein the compound has formula (V):

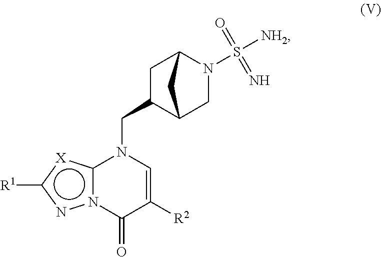

wherein R1, R2 and X are as defined in Clause 164 or Clause 164a.

167. The compound of any of Clauses 164 to 166, wherein X is N.

168. The compound of any of Clauses 164 to 166, wherein X is C(CH₂)ₘR5, and R5 is as defined in Clause 164 or Clause 164a.

169. The compound of any of Clauses 164 to 166 or 168, wherein m is 0.

170. The compound of any of Clauses 164 to 166 or 168, wherein m is 1.

171. The compound of any of Clauses 164 to 166, 168 or 169, wherein X is CH.

172. The compound of any of Clauses 164 to 166, 168 or 169, wherein X is C-halo, optionally wherein halo is Cl or F (e.g. F).

173. The compound of any of Clauses 164 to 166, 168 or 169, wherein X is CR5, and R5 is OMe. 174. The compound of any of Clauses 164 to 166, 168 or 169, wherein X is CR5, and R5 is CN.

175. The compound of any of Clauses 164 to 166, 168 or 169, wherein X is CR5, and R5 is Me.

176. The compound of any of Clauses 164 to 166, 168, 169 or 171, wherein X is CR5, and R5 is 2H.

176a. The compound of any of Clauses 164 to 166, 168, 169 or 171, wherein X is CR5, and R5 is 1H.

177. The compound of any of Clauses 164 to 176a, wherein R1 is H or halo.

178. The compound of any of Clauses 164 to 176a, wherein R1 is C1-C6 alkyl or C1-C6 haloalkyl.

178a. The compound of any of Clauses 164 to 176a or 178, wherein R1 is C1-C6 alkyl.

179. The compound of any of Clauses 164 to 176a, wherein R1 is C3-C6 cycloalkyl (e.g. cyclopropyl), optionally substituted with one or more (e.g., 1, 2, 3, or 4) substituents independently selected from C1-C3 alkyl, C3-C6 cycloalkyl and halo.

179a. The compound of any of Clauses 164 to 176a or 179, wherein R1 is C3-C6 cycloalkyl.

179b. The compound of any of Clauses 164 to 176a, 179 or 179a, wherein R1 is cyclopropyl.

179c. The compound of any of Clauses 164 to 176a, wherein R1 is cyclopropyl or ethyl.

180. The compound of any of Clauses 164 to 176a, wherein R1 is C1-C6 alkoxyl or C1-C6 haloalkoxyl.

180a. The compound of any of Clauses 164 to 176a or 180, wherein R1 is C1-C6 alkoxyl.

180b. The compound of any of Clauses 164 to 177, wherein R1 is H.

181. The compound of any of Clauses 164 to 176a, wherein R1 is H or is selected from:

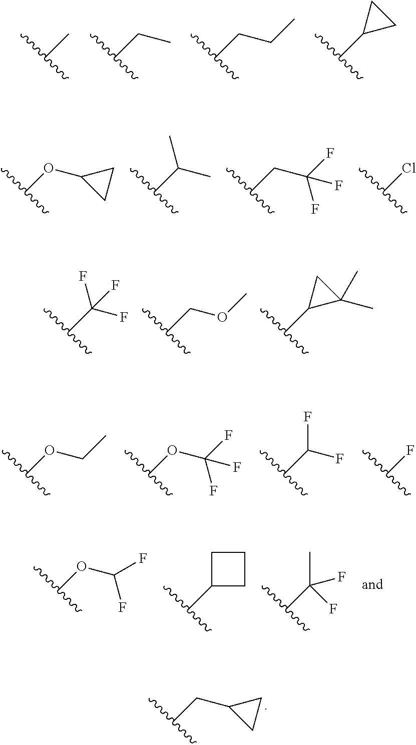

182. The compound of any of Clauses 164 to 176a or 181, wherein R1 is H or is selected from:

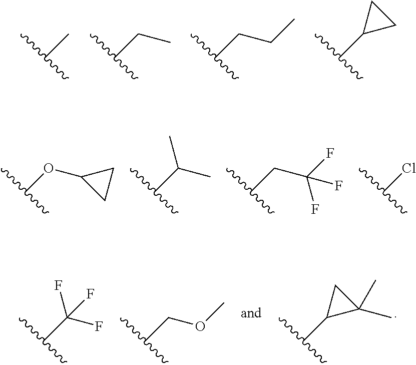

183. The compound of any of Clauses 164, 165, 166 or 168 to 170, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

184. The compound of any of Clauses 164 to 166, 168 to 170 or 183, wherein R5 and R1 taken together with the atoms to which they are attached form a 5-6 membered cycloalkyl.

185. The compound of any of Clauses 164 to 166, 168 to 170 or 183, wherein R5 and R1 taken together with the atoms to which they are attached form a 5-6 membered heterocycloalkyl having one or more (e.g., 1 or 2) ring heteroatoms independently selected from N, O, and S.

186. The compound of any of Clauses 164, 165, 166, 168 to 170 or 183, wherein R5 and R1 taken together with the atoms to which they are attached form a phenyl, optionally substituted with one or more (e.g., 1, 2, 3, or 4) substituents independently selected from C1-C6 alkoxyl.

187. The compound of any of Clauses 164, 165, 166, 168 to 170 or 183, wherein R5 and R1 taken together with the atoms to which they are attached form a 5-6 membered heteroaryl having one or more (e.g., 1 or 2) ring heteroatoms independently selected from N, O, and S.

188. The compound of any of Clauses 164, 165, 166, 168 to 170 or 183, wherein the compound is selected from any one of the following structures:

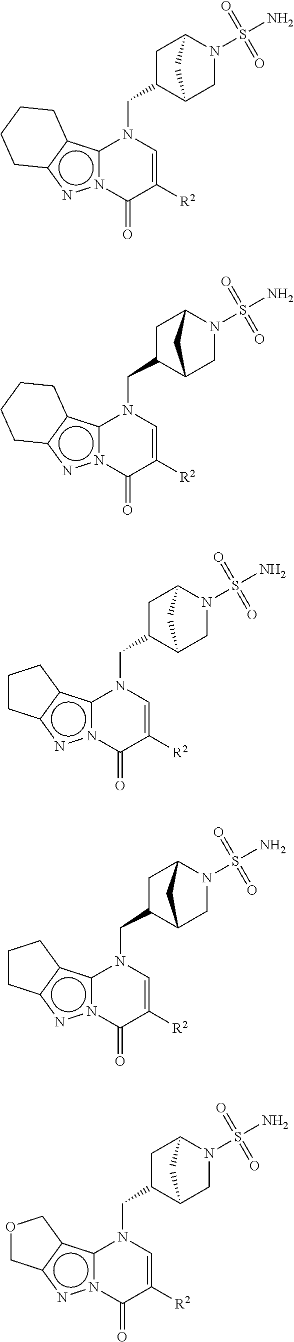

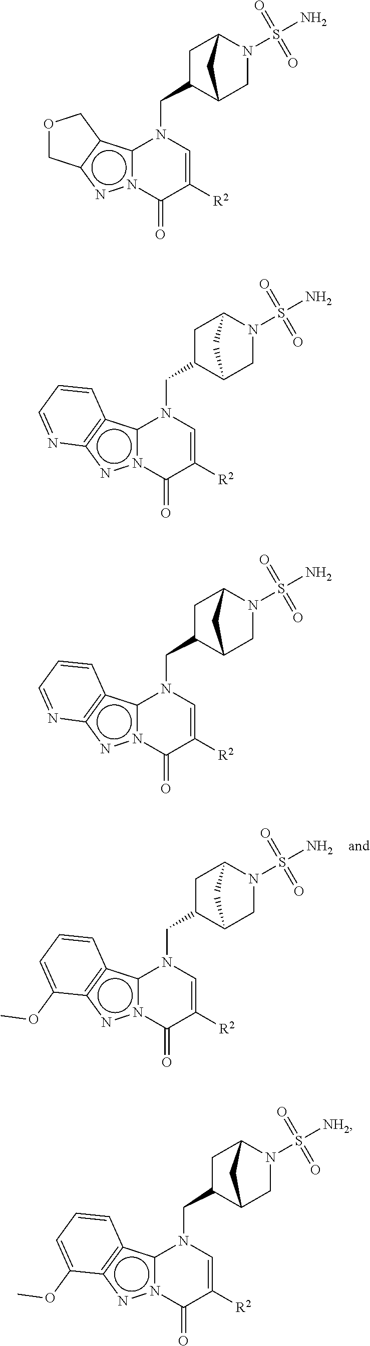

wherein R2 is as defined in Clause 164.

189. The compound of any of Clauses 164 to 188, wherein R2 is C1-C6 alkyl or C1-C6 haloalkyl.

189a. The compound of any of Clauses 164 to 189, wherein R2 is C1-C6 alkyl.

189b. The compound of any of Clauses 164 to 189a, wherein R2 is C1-C3 alkyl.

189c. The compound of any of Clauses 164 to 189b, wherein R2 is isopropyl.

190. The compound of any of Clauses 164 to 188, wherein R2 is C3-C6 cycloalkyl, optionally substituted with one or more (e.g., 1, 2, 3, or 4) halo.

191. The compound of any of Clauses 164 to 188, wherein R2 is phenyl, optionally substituted with one or more (e.g., 1, 2, 3, or 4) halo.

192. The compound of any of Clauses 164 to 188, wherein R2 is 6-membered heteroaryl having one or more (e.g. 1, 2, 3, or 4) ring heteroatoms independently selected from N, O, and S, wherein the heteroaryl is optionally substituted with one or more (e.g., 1, 2, 3, or 4) halo.

193. The compound of any of Clauses 164 to 188, wherein R2 is selected from:

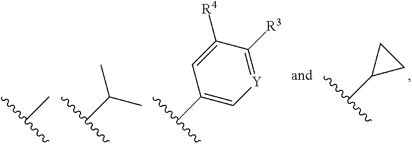

wherein Y is CH or N; R3 is H or halo; and R4 is H or halo (optionally halo is F).

194. The compound of any of Clauses 164 to 188 or 193, wherein R2 is selected from:

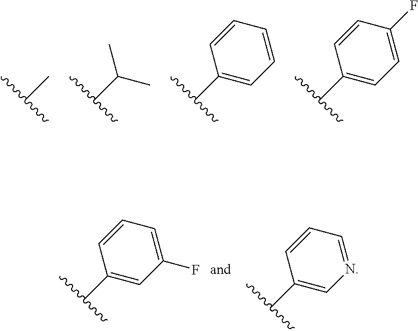

195. The compound of Clause 164, wherein the compound is selected from any one of the following structures:

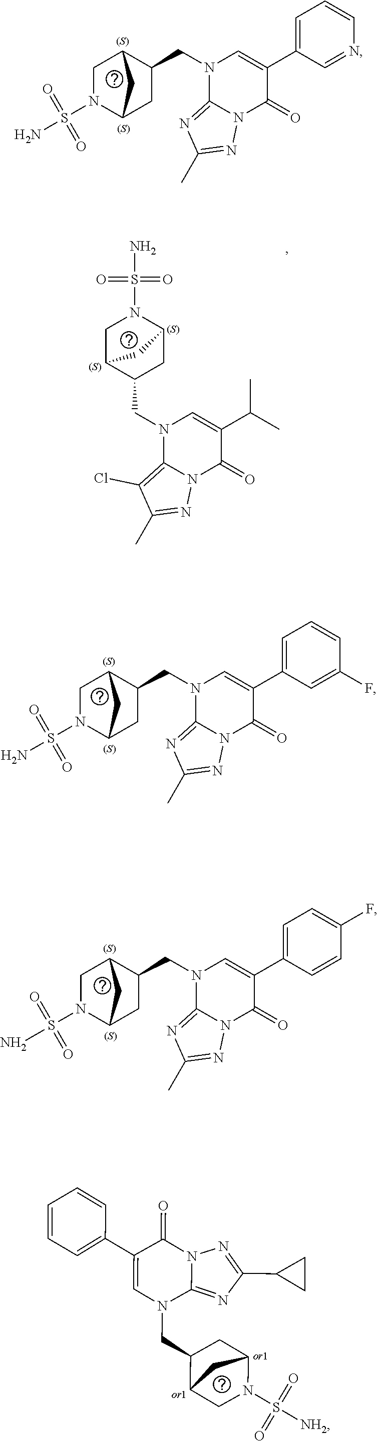

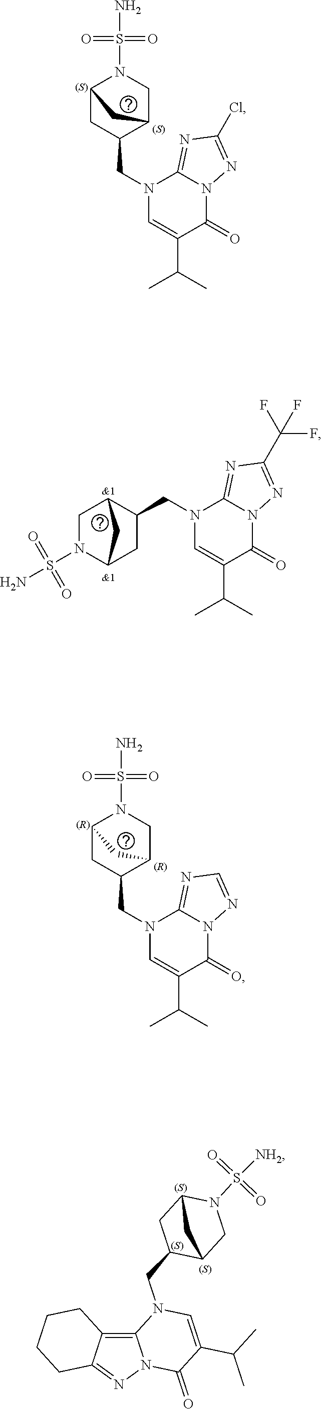

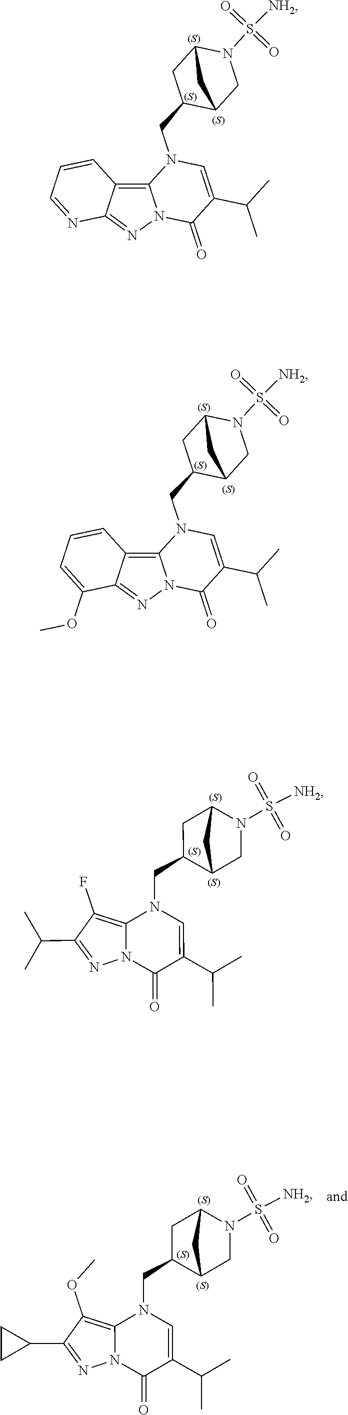

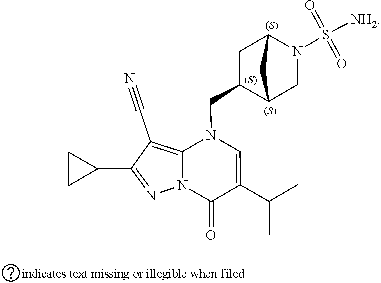

196. The compound of any of Clauses 164, 164a or 195, wherein the compound is selected from any one of the following structures:

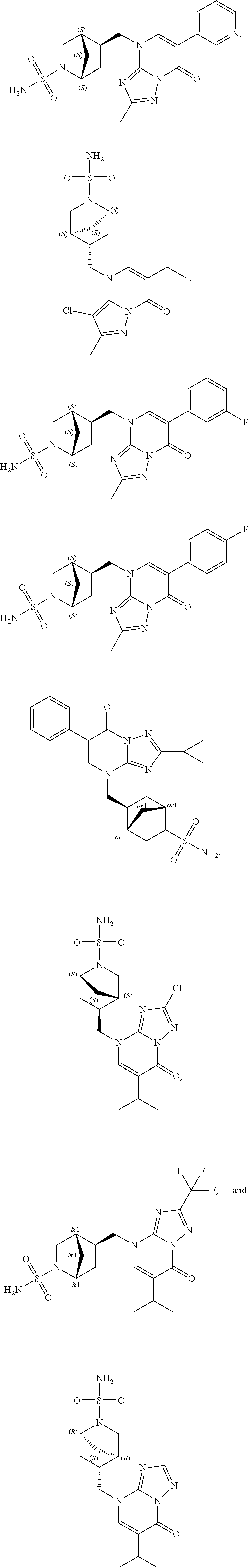

197. A compound of formula (VI) or (VII) or a pharmaceutically acceptable salt or solvate thereof:

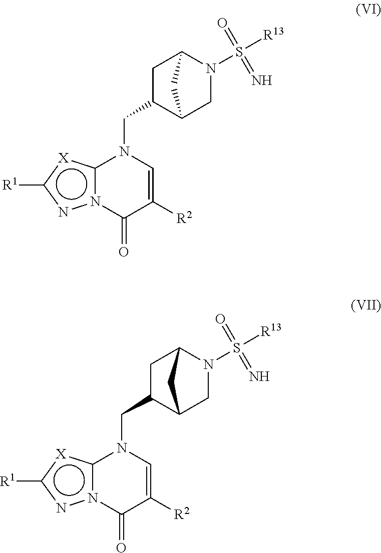

wherein:
   X is N or C(CH₂)ₘR5;
   R5 is selected from H, halo, Me, CN, and OMe;
   R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
   R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
   R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
   m is 0 or 1; and
   R13 is C1-C3 alkyl, C1-C3 haloalkyl, or C3-C6 cycloalkyl, wherein cycloalkyl is optionally substituted with one or more (e.g., 1, 2, 3, or 4) halo.

197a. The compound of Clause 197, wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered cycloalkyl or heterocycloalkyl having one or more (e.g., 1 or 2) ring heteroatoms independently selected from N, O, and S; and
R13 is methyl.

198. The compound of Clause 197 or Clause 197a, wherein the compound has formula (VI):

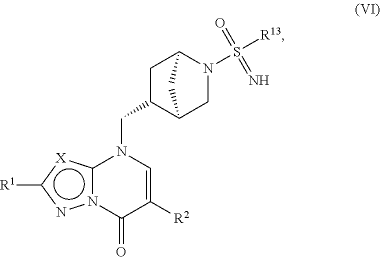

wherein R1, R2, X and R13 are as defined in Clause 197 or Clause 197a.

199. The compound of Clause 197 or Clause 197a, wherein the compound has formula (VII):

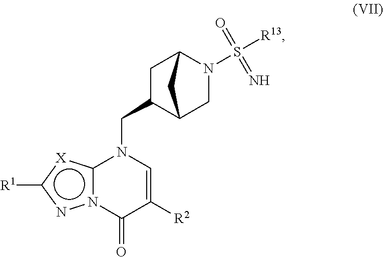

wherein R1, R2, X and R13 are as defined in Clause 197 or Clause 197a.

200. The compound of any of Clauses 197 to 199, wherein X is N.

201. The compound of any of Clauses 197 to 199, wherein X is C(CH₂)ₘR5, and R5 is as defined in Clause 197 or Clause 197a.

202. The compound of any of Clauses 197 to 199 or 201, wherein m is 0.

203. The compound of any of Clauses 197 to 199 or 201, wherein m is 1,

204. The compound of any of Clauses 197 to 199, 201 or 202, wherein X is CH.

205. The compound of any of Clauses 197 to 199, 201 or 202, wherein X is C-halo, optionally wherein halo is Cl or F (e.g. F).

206. The compound of any of Clauses 197 to 199, 201 or 202, wherein X is CR5, and R5 is OMe.

207. The compound of any of Clauses 197 to 199, 201 or 202, wherein X is CR5, and R5 is CN.

208. The compound of any of Clauses 197 to 199, 201 or 202, wherein X is CR5, and R5 is Me.

209. The compound of any of Clauses 197 to 199, 201, 202, 204, wherein X is CR5, and R5 is 2H.

209a. The compound of any of Clauses 197 to 199, 201, 202 or 204, wherein X is CR5, and R5 is 1H.

210. The compound of any of Clauses 197 to 209a, wherein R1 is H or halo.

211. The compound of any of Clauses 197 to 209a, wherein R1 is C1-C6 alkyl or C1-C6 haloalkyl.

211a. The compound of any of Clauses 197 to 209a or 211, wherein R1 is C1-C6 alkyl.

212. The compound of any of Clauses 197 to 209a, wherein R1 is C3-C6 cycloalkyl (e.g. cyclopropyl), optionally substituted with one or more (e.g., 1, 2, 3, or 4) substituents independently selected from C1-C3 alkyl, C3-C6 cycloalkyl and halo.

212a. The compound of any of Clauses 197 to 209a or 212, wherein R1 is C3-C6 cycloalkyl.

212b. The compound of any of Clauses 197 to 209a, 212 or 212a, wherein R1 is cyclopropyl.

212c. The compound of any of Clauses 197 to 209a, wherein R1 is cyclopropyl or ethyl.

213. The compound of any of Clauses 197 to 209a, wherein R1 is C1-C6 alkoxyl or C1-C6 haloalkoxyl.

213a. The compound of any of Clauses 197 to 209a or 213, wherein R1 is C1-C6 alkoxyl.

214. The compound of any of Clauses 197 to 210, wherein R1 is H.

215. The compound of any of Clauses 197 to 209a, wherein R1 is H or is selected from:

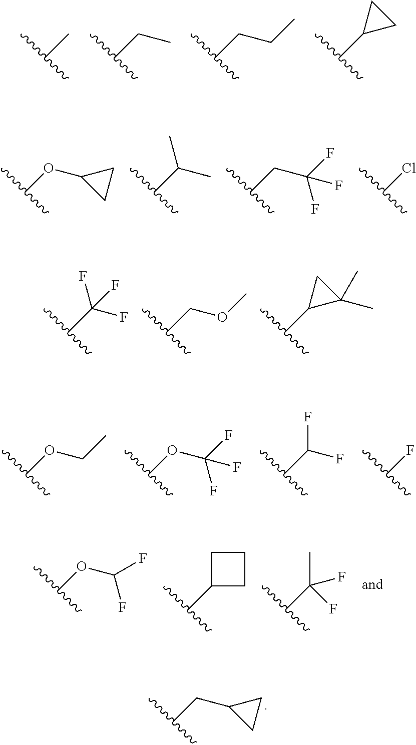

216. The compound of any of Clauses 197 to 209a or 215, wherein R1 is H or is selected from;

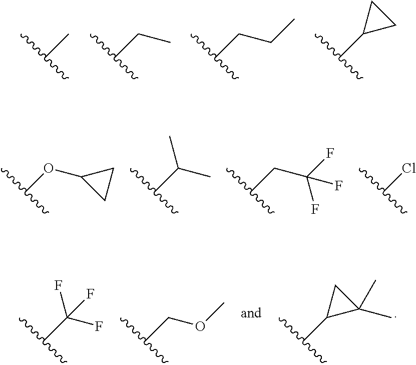

217. The compound of any of Clauses 197, 198, 199 or 201 to 203, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

218. The compound of any of Clauses 197 to 199, 201 to 203 or 217, wherein R5 and R1 taken together with the atoms to which they are attached form a 5-6 membered cycloalkyl.

219. The compound of any of Clauses 197 to 199, 201 to 203 or 217, wherein R5 and R taken together with the atoms to which they are attached form a 5-6 membered heterocycloalkyl having one or more (e.g., 1 or 2) ring heteroatoms independently selected from N, O, and S.

220. The compound of any of Clauses 197, 198, 199, 201 to 203 or 217, wherein R5 and R1 taken together with the atoms to which they are attached form a phenyl, optionally substituted with one or more (e.g., 1, 2, 3, or 4) substituents independently selected from C1-C6 alkoxyl.

221. The compound of any of Clauses 197, 198, 199, 201 to 203 or 217, wherein R5 and R1 taken together with the atoms to which they are attached form a 5-6 membered heteroaryl having one or more (e.g., 1 or 2) ring heteroatoms independently selected from N, O, and S.

222. The compound of any of Clauses 197, 198, 199, 201 to 203 or 217, wherein the compound is selected from any one of the following structures:

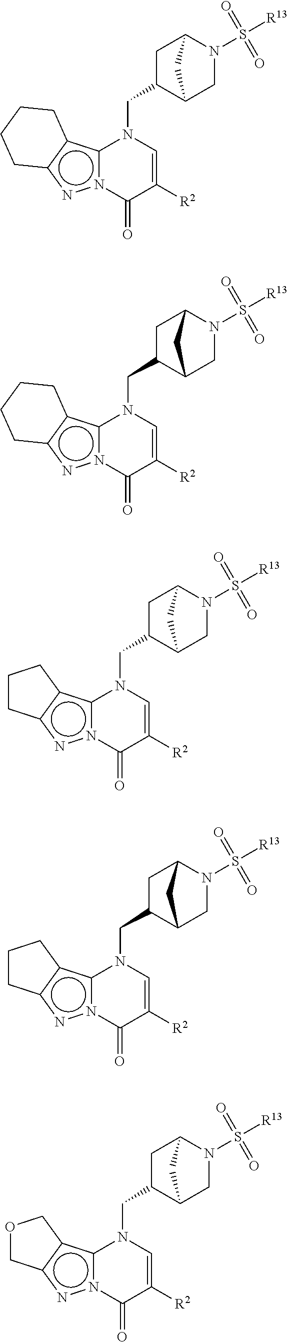

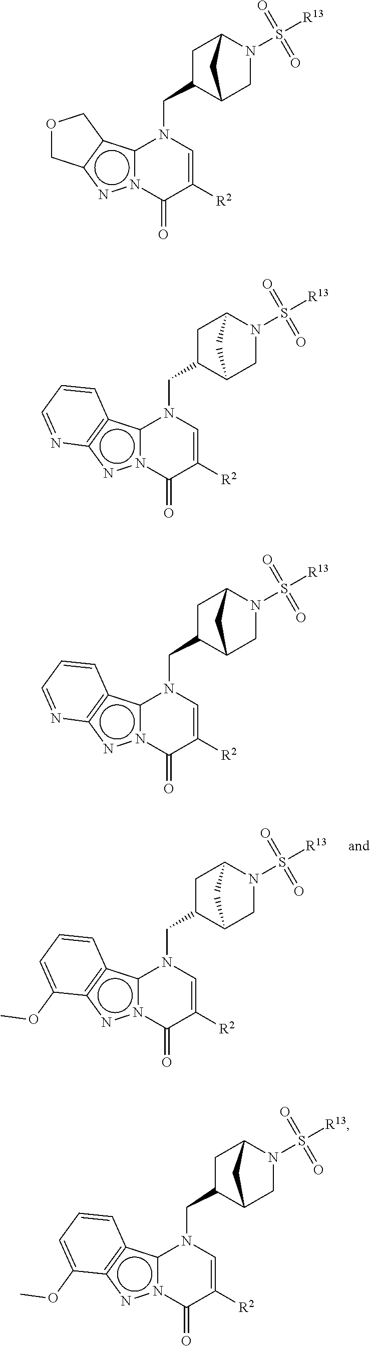

wherein R2 and R13 are as defined in Clause 197 or Clause 197a.

223. The compound of any of Clauses 197 to 222, wherein R2 is C1-C6 alkyl or C1-C6 haloalkyl.

224. The compound of any of Clauses 197 to 223, wherein R2 is C1-C6 alkyl.

225. The compound of any of Clauses 197 to 224, wherein R2 is C1-C3 alkyl.

226. The compound of any of Clauses 197 to 225, wherein R2 is isopropyl.

227. The compound of any of Clauses 197 to 222, wherein R2 is C3-C6 cycloalkyl, optionally substituted with one or more (e.g., 1, 2, 3, or 4) halo.

228. The compound of any of Clauses 197 to 222, wherein R2 is phenyl, optionally substituted with one or more (e.g., 1, 2, 3, or 4) halo.

229. The compound of any of Clauses 197 to 222, wherein R2 is 6-membered heteroaryl having one or more (e.g. 1, 2, 3, or 4) ring heteroatoms independently selected from N, O, and S, wherein the heteroaryl is optionally substituted with one or more (e.g., 1, 2, 3, or 4) halo.

230. The compound of any of Clauses 197 to 222, wherein R2 is selected from:

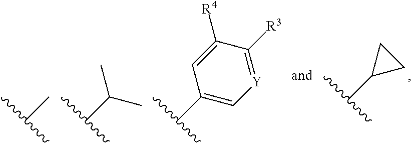

wherein Y is CH or N; R3 is H or halo; and R4 is H or halo (optionally halo is F).

231. The compound of any of Clauses 197 to 222 or 230, wherein R2 is selected from:

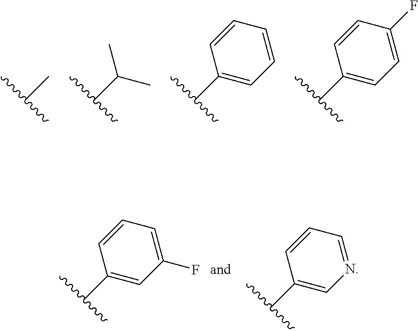

232. The compound of any of Clauses 197 or 198 to 231, wherein R13 is C1-C3 alkyl (e.g. C¹H₃ or C²H₃) or C1-C3 haloalkyl (e.g. CF₃ or CHF₂).

233. The compound of any of Clauses 197 to 232, wherein R13 is Me (e.g. C₁H³ or C²H₃).

234. The compound of any of Clauses 197 or 198 to 231, wherein R13 is C3-C6 cycloalkyl (e.g. cyclopropyl), optionally substituted with one or more (e.g., 1, 2, 3, or 4) halo.

235. The compound of any of Clauses 197, 198 to 231 or 234, wherein R13 is C3-C6 cycloalkyl.

235a. The compound of any of Clauses 197, 198 to 231, 234 or 235, wherein R13 is cyclopropyl.

235b. The compound of any of Clauses 197 to 231, wherein R13 is selected from C3-C6 cycloalkyl and C1-C3 alkyl.

235c. The compound of any of Clauses 197 to 231 or 235b, wherein R13 is selected from cyclopropyl, methyl (e.g. C₁H³ or C²H₃), ethyl and isopropyl.

236. The compound of any of Clauses 197 or 198 to 231, wherein the compound has a structure selected from:

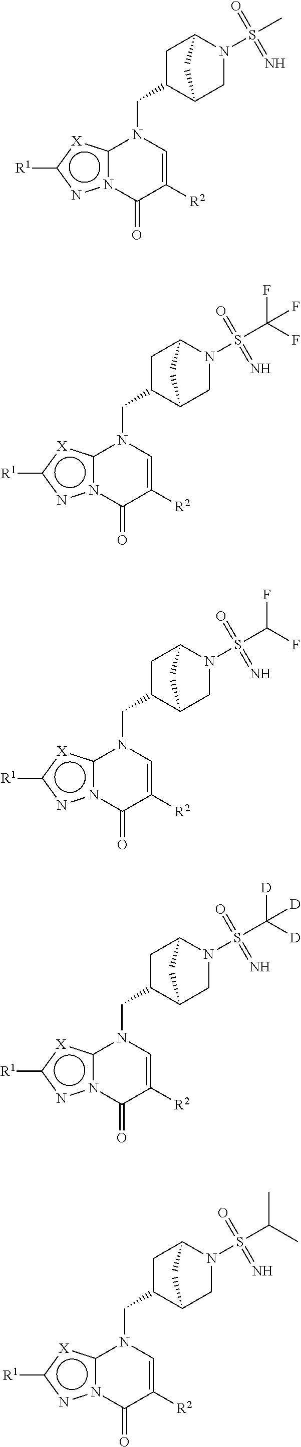

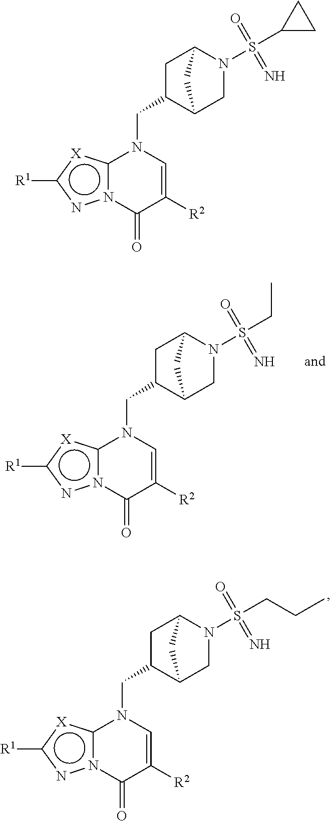

wherein X, R1, R2 are as defined in Clause 197 or Clause 197a.

237. The compound of any of Clauses 197 or 198 to 231, wherein the compound has a structure selected from:

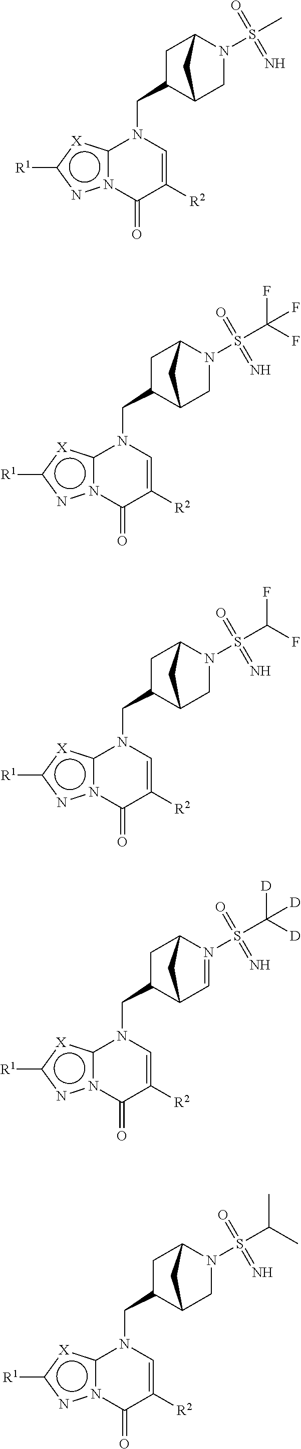

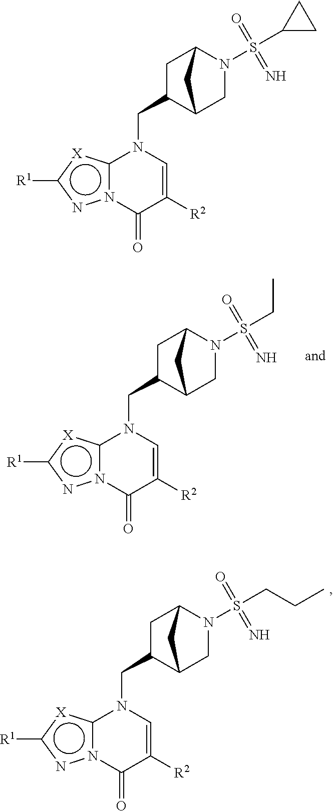

wherein X, R1, R2 are as defined in Clause 197 or Clause 197a.

238. The compound of Clause 197 or Clause 197a, wherein the compound is selected from:

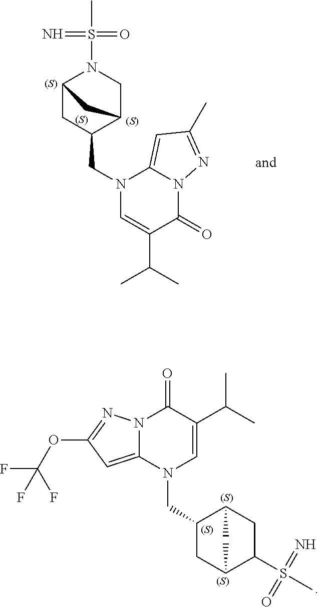

238a. A compound of formula (I) or a pharmaceutically acceptable salt or solvate thereof:

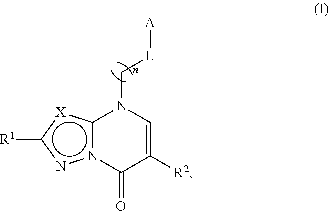

wherein:
   X is N or CH;
   R1 is selected from C1-C6 alkyl, C1-C6 haloalkyl and C3-C6 cycloalkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo;
   R2 is selected from C1-C6 alkyl and C1-C6 haloalkyl;
   n is 1, 2, or 3;
   L is phenyl, wherein the phenyl is optionally substituted with one or more (e.g., 1, 2, 3, or 4) substituents independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S;
   each R11 is independently selected from H and C1-C3 alkyl;
   A is selected from —SO₂NH2 and —S(O)(NH)R13; and
   R13 is C1-C3 alkyl, C1-C3 haloalkyl, or C3-C6 cycloalkyl, wherein cycloalkyl is optionally substituted with one or more (e.g., 1, 2, 3, or 4) halo.

238b. The compound of Clause 238a, wherein X is N.

238c. The compound of Clause 238a or Clause 238b, wherein R1 is selected from C1-C6 alkyl and C3-C6 cycloalkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo.

238d. The compound of any of Clauses 238a to 238c, wherein R1 is selected from C1-C6 alkyl and C3-C6 cycloalkyl.

238e. The compound of any of Clauses 238a to 238d, wherein R1 is selected from C1-C3 alkyl and C3-C6 cycloalkyl.

238f. The compound of any of Clauses 238a to 238e, wherein R1 is selected from ethyl and cyclopropyl.

238g. The compound of any of Clauses 238a to 238f, wherein R1 is cyclopropyl.

238h. The compound of any of Clauses 238a to 238g, wherein R2 is C1-C6 alkyl.

238i. The compound of any of Clauses 238a to 238h, wherein R2 is C1-C3 alkyl.

238j. The compound of any of Clauses 238a to 238i, wherein R2 is isopropyl.

238k. The compound of any of Clauses 238a to 238j, wherein n is 1.

238l. The compound of any of Clauses 238a to 238k, wherein L is phenyl, and the phenyl is optionally substituted with one or more (e.g., 1, 2, 3, or 4) substituents independently selected from halo, C1-C6 alkyl and C1-C6 haloalkyl.

238 m. The compound of any of Clauses 238a to 238l, wherein L is phenyl, and the phenyl is optionally substituted with one or more (e.g., 1, 2, 3, or 4) substituents independently selected from halo and C1-C6 alkyl.

238n. The compound of any of Clauses 238a to 238 m, wherein L is phenyl, and the phenyl is optionally substituted with one or more (e.g., 1, 2, 3, or 4) substituents independently selected from halo and C1-C3 alkyl.

238o. The compound of any of Clauses 238a to 238n, wherein L is phenyl, and the phenyl is optionally substituted with one or more (e.g., 1, 2, 3, or 4) substituents independently selected from halo and methyl.

238p. The compound of any of Clauses 238a to 2380, wherein L is phenyl, and the phenyl is optionally substituted with one or more (e.g., 1, 2, 3, or 4) substituents independently selected from F and methyl.

238q. The compound of any of Clauses 238a to 238p, wherein L is selected from:

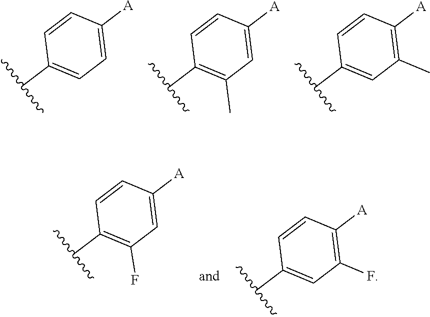

238r. The compound of any of Clauses 238a to 238q, wherein L is phenyl.

238s. The compound of any of Clauses 238a to 238r, wherein A is —S(O)(NH)R13.

238t. The compound of any of Clauses 238a to 238s, wherein R13 is C1-C3 alkyl (e.g. C₁H³ or C²H₃) or C3-C6 cycloalkyl, wherein cycloalkyl is optionally substituted with one or more (e.g., 1, 2, 3, or 4) halo.

238 u. The compound of any of Clauses 238a to 238t, wherein R13 is C1-C3 alkyl or C3-C6 cycloalkyl.

238v. The compound of any of Clauses 238a to 238 u, wherein R13 is selected from methyl (e.g., C¹H₃ or C²H₃), ethyl, isopropyl and cyclopropyl.

238w. The compound of any of Clauses 238a to 238 u, wherein R13 is C1-C3 alkyl.

238x. The compound of any of Clauses 238a to 238w, wherein R13 is methyl (e.g., C₁H³ or C²H₃).

238y. The compound of Clause 238a, wherein the compound is selected from the group consisting of the following structures:

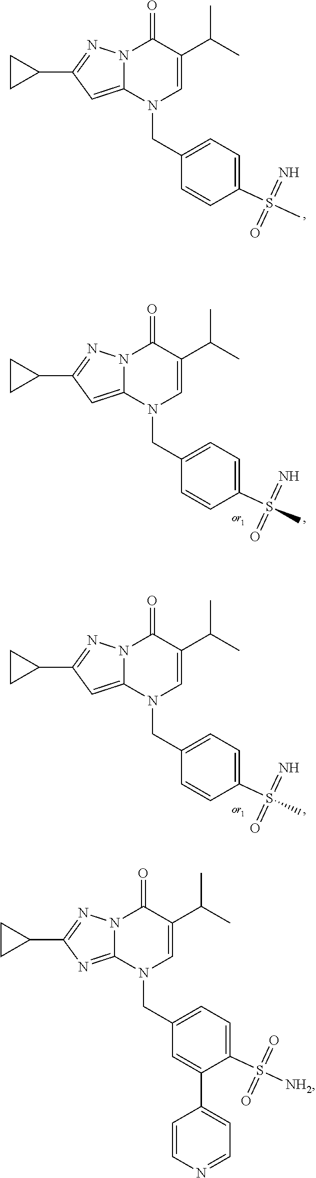 [SMILES: CC(C)c1cn(Cc2ccc(S(C)(=N)=O)cc2)c2cc(C3CC3)nn2c1=O ; CC(C)c1cn(Cc2ccc([S@](C)(=N)=O)cc2)c2cc(C3CC3)nn2c1=O ; CC(C)c1cn(Cc2ccc([S@@](C)(=N)=O)cc2)c2cc(C3CC3)nn2c1=O ; CC(C)c1cn(Cc2ccc(S(N)(=O)=O)c(-c3ccncc3)c2)c2nc(C3CC3)nn2c1=O]

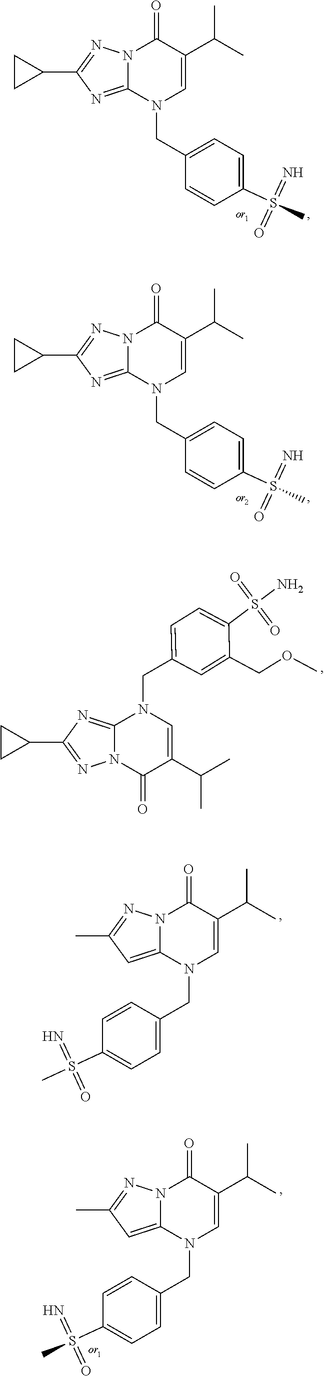

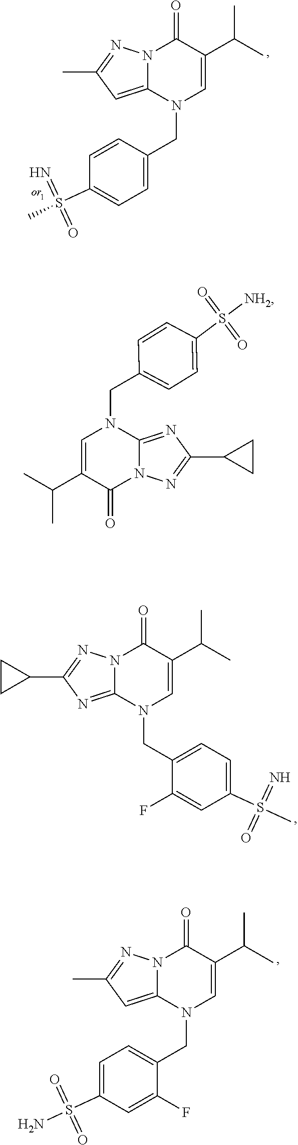

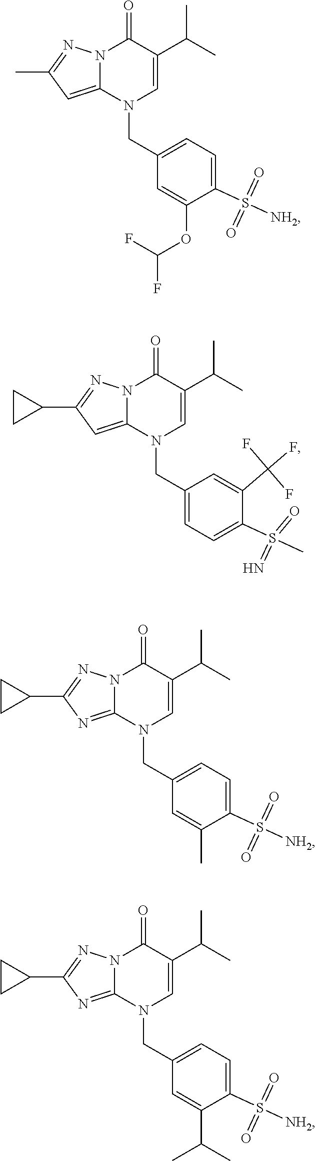 [SMILES: Cc1cc2n(Cc3ccc(S(N)(=O)=O)c(OC(F)F)c3)cc(C(C)C)c(=O)n2n1 ; CC(C)c1cn(Cc2ccc(S(C)(=N)=O)c(C(F)(F)F)c2)c2cc(C3CC3)nn2c1=O ; Cc1cc(Cn2cc(C(C)C)c(=O)n3nc(C4CC4)nc23)ccc1S(N)(=O)=O ; CC(C)c1cc(Cn2cc(C(C)C)c(=O)n3nc(C4CC4)nc23)ccc1S(N)(=O)=O]

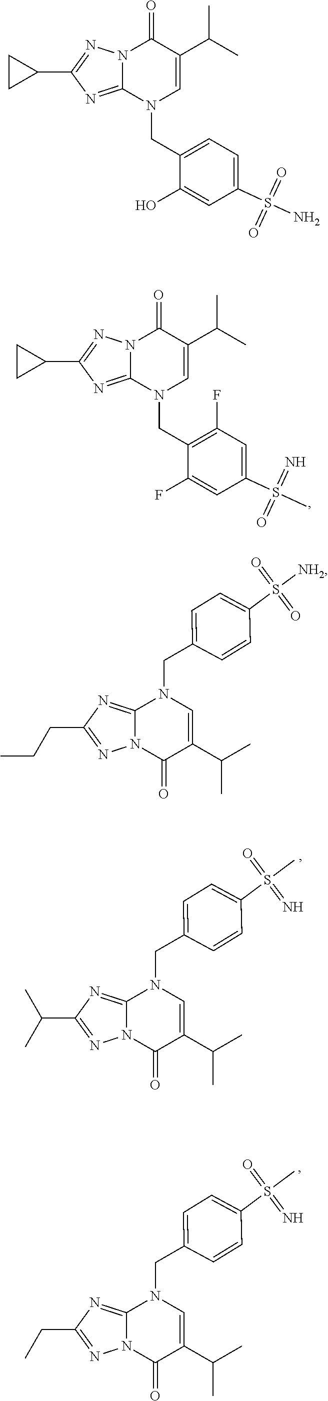

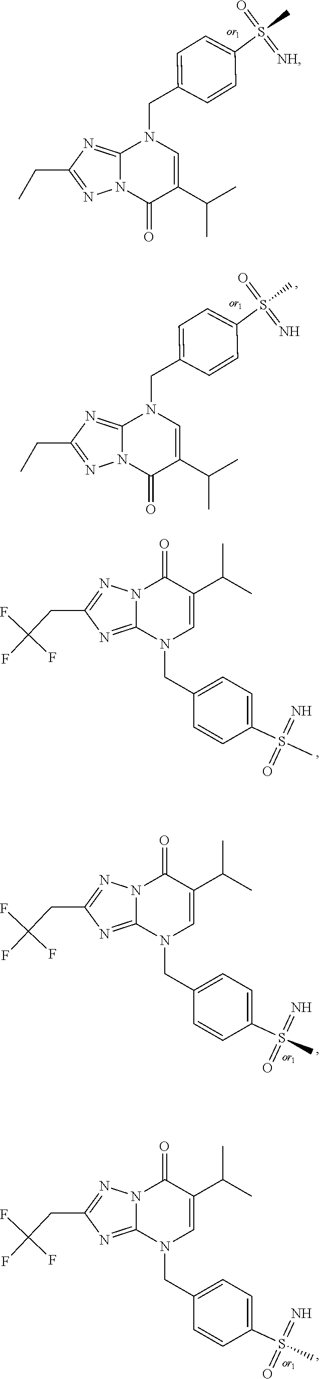

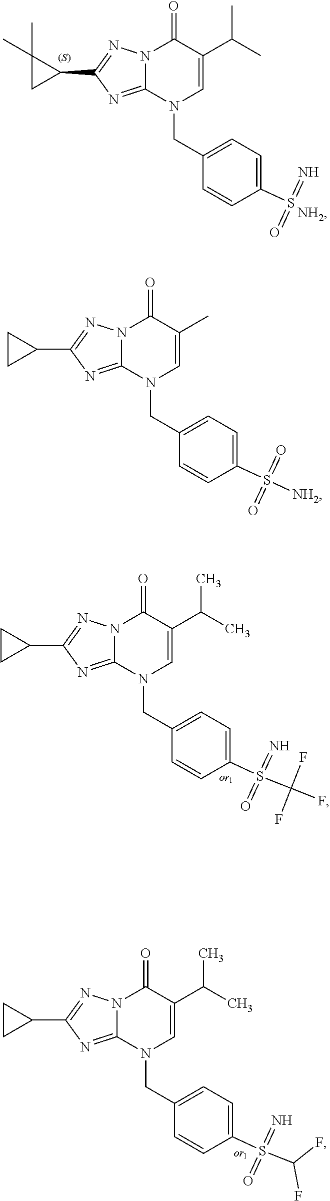

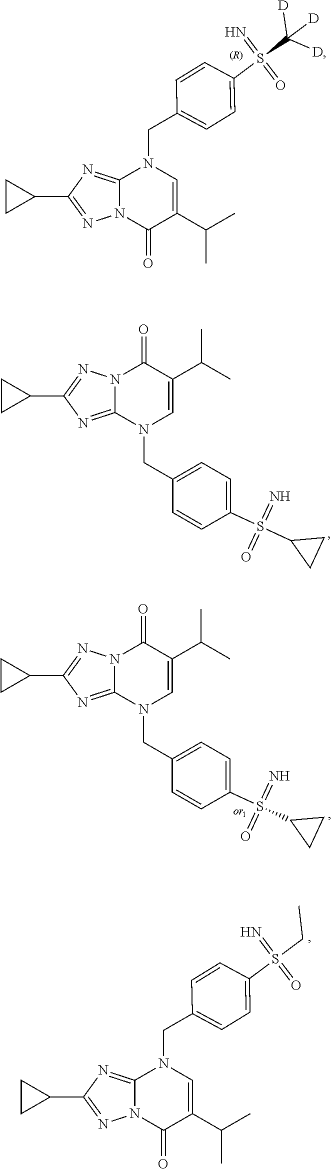

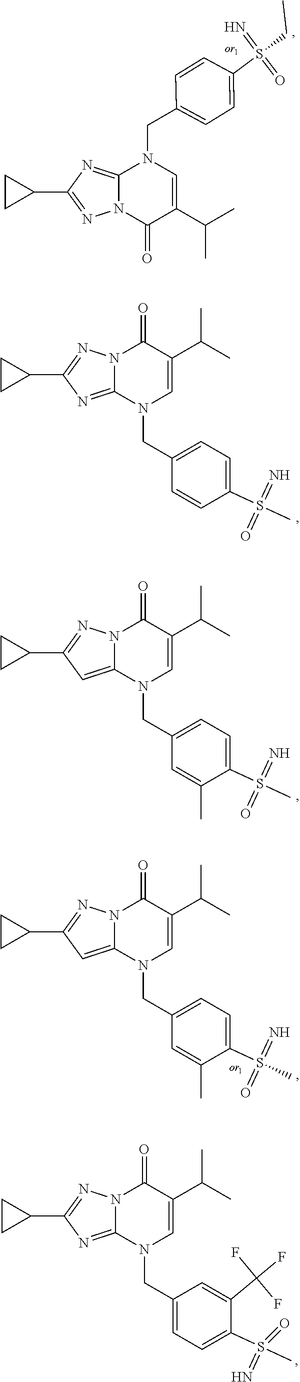

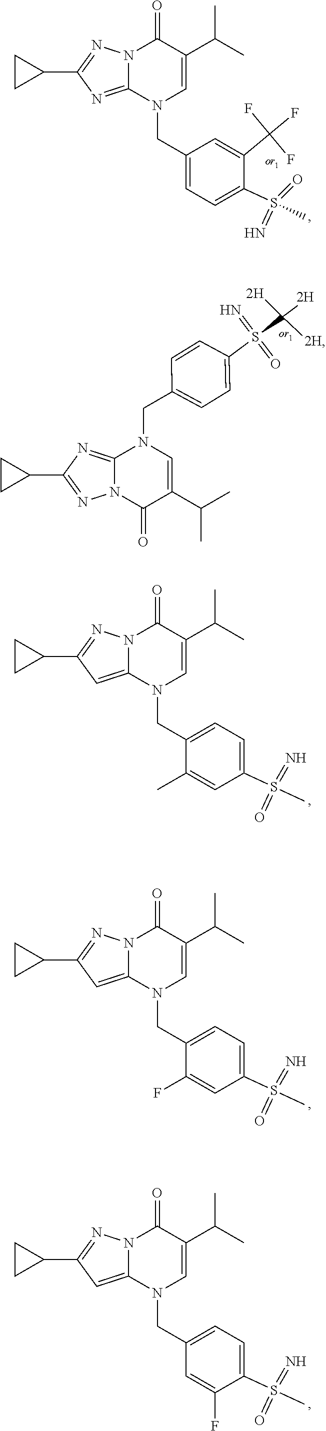

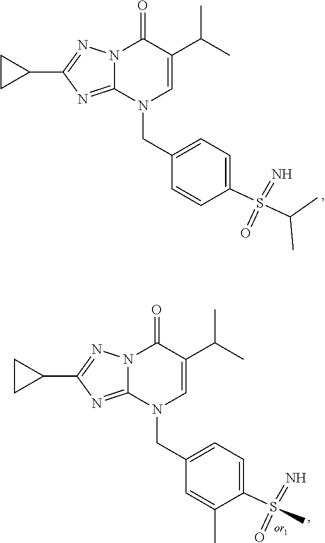 [SMILES: CC(C)c1cn(Cc2ccc(S(=N)(=O)C(C)C)cc2)c2nc(C3CC3)nn2c1=O ; Cc1cc(Cn2cc(C(C)C)c(=O)n3nc(C4CC4)nc23)ccc1[S@](C)(=N)=O]

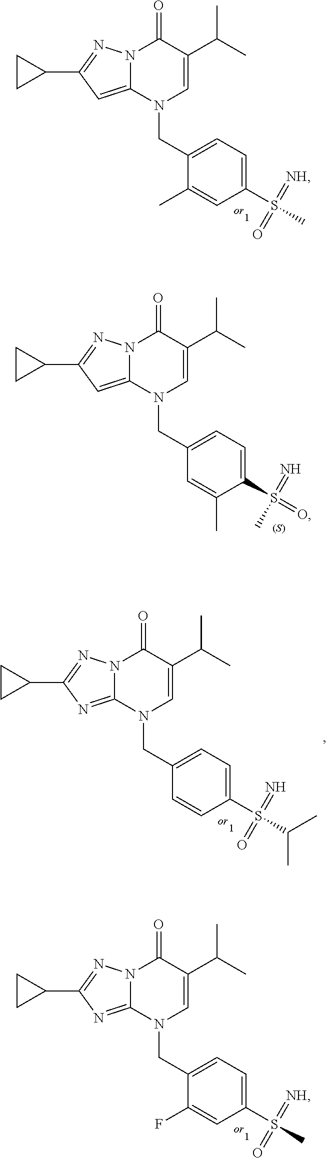

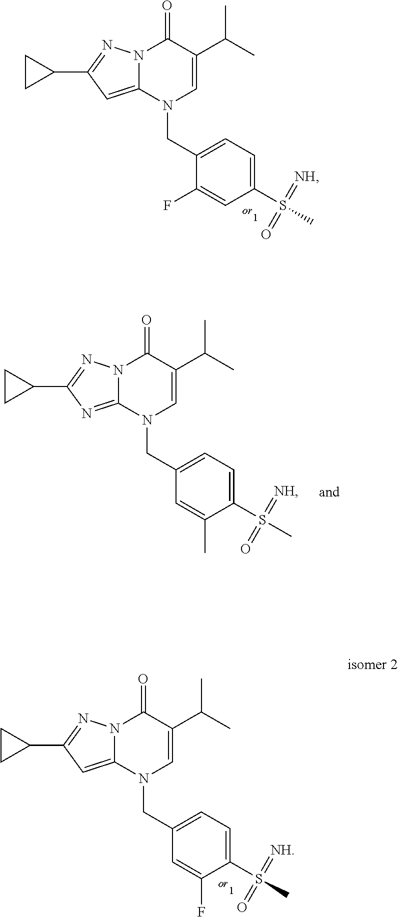 [SMILES: CC(C)c1cn(Cc2ccc([S@@](C)(=N)=O)cc2F)c2cc(C3CC3)nn2c1=O ; Cc1cc(Cn2cc(C(C)C)c(=O)n3nc(C4CC4)nc23)ccc1S(C)(=N)=O ; CC(C)c1cn(Cc2ccc([S@](C)(=N)=O)c(F)c2)c2cc(C3CC3)nn2c1=O]

238z. A compound of formula (VIII) or a pharmaceutically acceptable salt or solvate thereof:

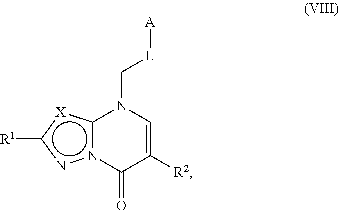

wherein:
   X is N or CH;
   R1 is selected from C1-C6 alkyl and C3-C6 cycloalkyl;
   R2 is C1-C6 alkyl;
   L is phenyl, wherein the phenyl is optionally substituted with one or more (e.g., 1, 2, 3, or 4) substituents independently selected from halo and C1-C6 alkyl;
   A is selected from —SO₂NH₂ and —S(O)(NH)R13; and
   R13 is C1-C3 alkyl or C3-C6 cycloalkyl.

238aa. The compound of Clause 238z, wherein X is N.

238bb. The compound of Clause 238z or Clause 238aa, wherein R1 is selected from C1-C3 alkyl and C3-C6 cycloalkyl.

238cc. The compound of any of Clauses 238z to 238bb, wherein R1 is selected from ethyl and cyclopropyl.

238dd. The compound of any of Clauses 238z to 238cc, wherein R1 is cyclopropyl.

238ee. The compound of any of Clauses 238z to 238dd, wherein R2 is C1-C3 alkyl.

238ff. The compound of any of Clauses 238z to 238ee, wherein R2 is isopropyl.

238gg. The compound of any of Clauses 238z to 238ff, wherein L is phenyl, and the phenyl is optionally substituted with one or more (e.g., 1, 2, 3, or 4) substituents independently selected from halo and C1-C3 alkyl.

238hh. The compound of any of Clauses 238z to 238gg, wherein L is phenyl, and the phenyl is optionally substituted with one or more (e.g., 1, 2, 3, or 4) substituents independently selected from halo and methyl.

238ii. The compound of any of Clauses 238z to 238hh, wherein L is phenyl, and the phenyl is optionally substituted with one or more (e.g., 1, 2, 3, or 4) substituents independently selected from F and methyl.

238j. The compound of any of Clauses 238z to 238ii, wherein L is selected from the following structures:

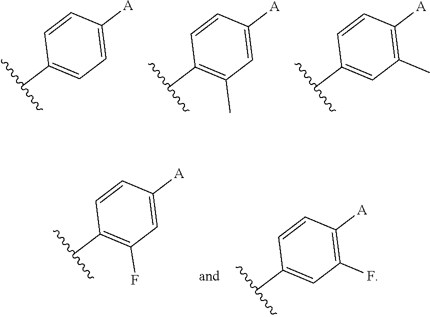

238kk. The compound of any of Clauses 238z to 238j, wherein L is phenyl.

238ll. The compound of any of Clauses 238z to 238kk, wherein A is —S(O)(NH)R13.

238 mm. The compound of any of Clauses 238z to 238ll, wherein R13 is selected from methyl (e.g., C₁H³ or C²H₃), ethyl, isopropyl and cyclopropyl.

238nn. The compound of any of Clauses 238z to 238ll, wherein R13 is C1-C3 alkyl.

23800. The compound of any of Clauses 238z to 238nn, wherein R13 is methyl.

238pp. The compound of Clause 238a, wherein the compound is selected from the group consisting of the following structures:

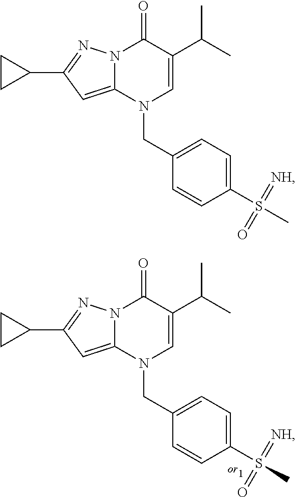 [SMILES: CC(C)c1cn(Cc2ccc(S(C)(=N)=O)cc2)c2cc(C3CC3)nn2c1=O ; CC(C)c1cn(Cc2ccc([S@](C)(=N)=O)cc2)c2cc(C3CC3)nn2c1=O]

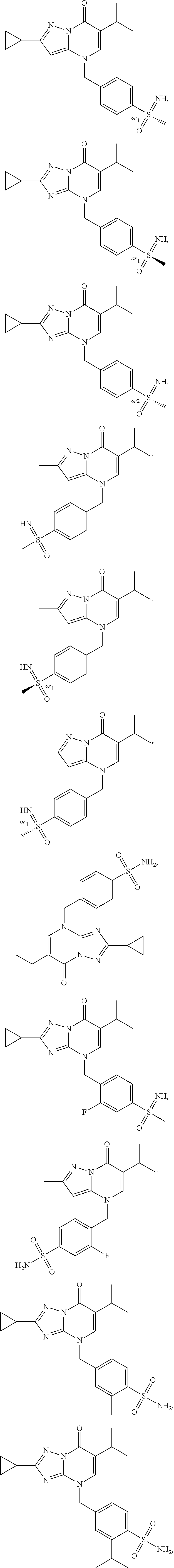

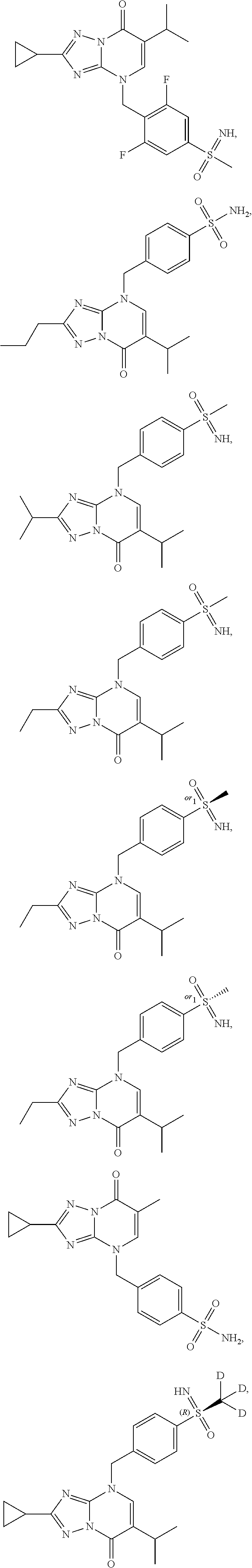

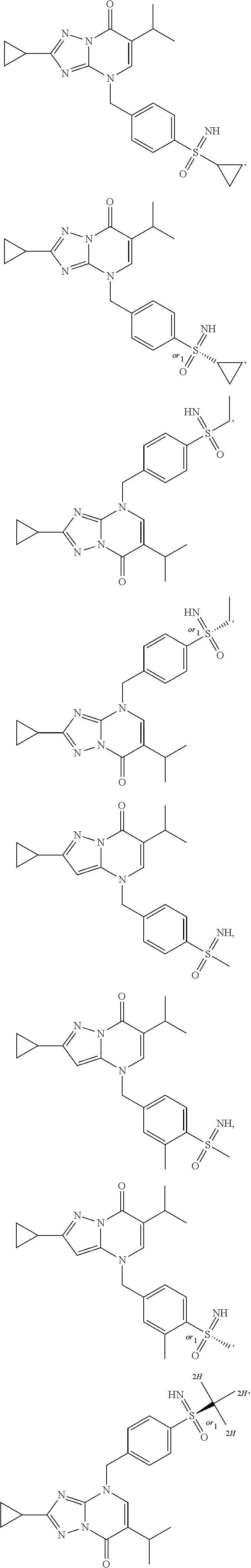

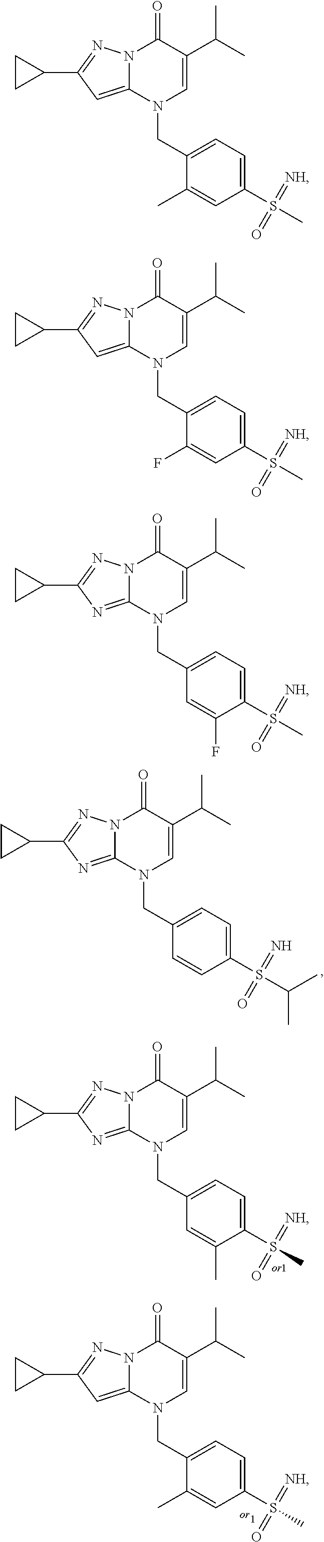

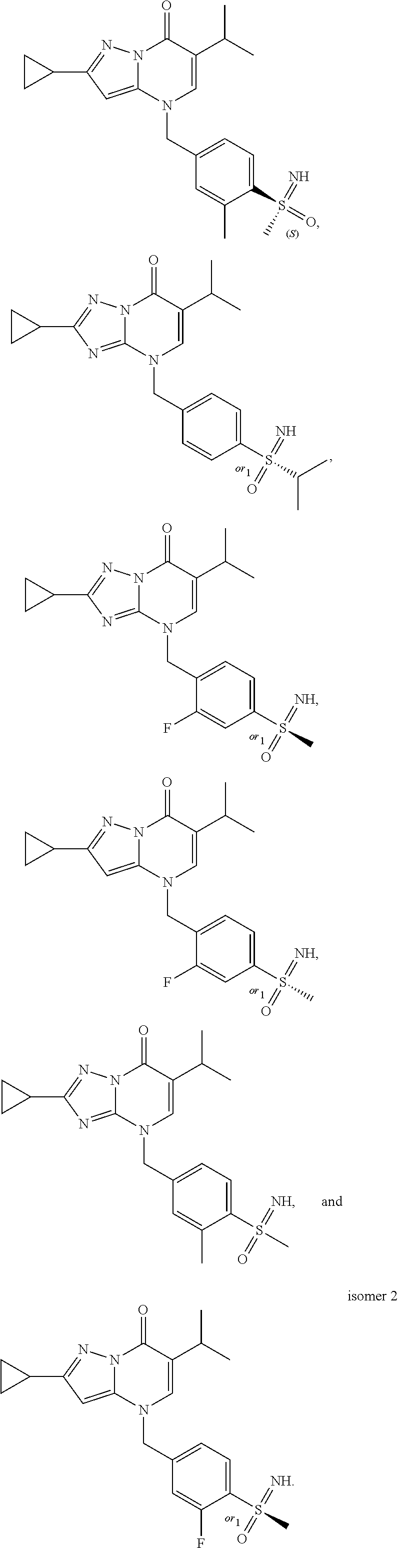

239. A compound selected from a compound of:
(i) the group of compounds shown in Table 1;
(ii) the group of compounds of Table 1 having a pEC₅₀ for ENPP1 human enzyme of >5;
(iii) the group of compounds of Table 1 having a pEC₅₀ for ENPP1 human enzyme of >6;
(iv) the group of compounds of Table 1 having a pEC₅₀ for ENPP1 human enzyme of >7; or
(v) the group of compounds of Table 1 having a pEC₅₀ for ENPP1 human enzyme of >8 or a pharmaceutically acceptable salt or solvate thereof.

239a. A compound, or a pharmaceutically acceptable salt or solvate thereof, selected from the group of compounds consisting of:

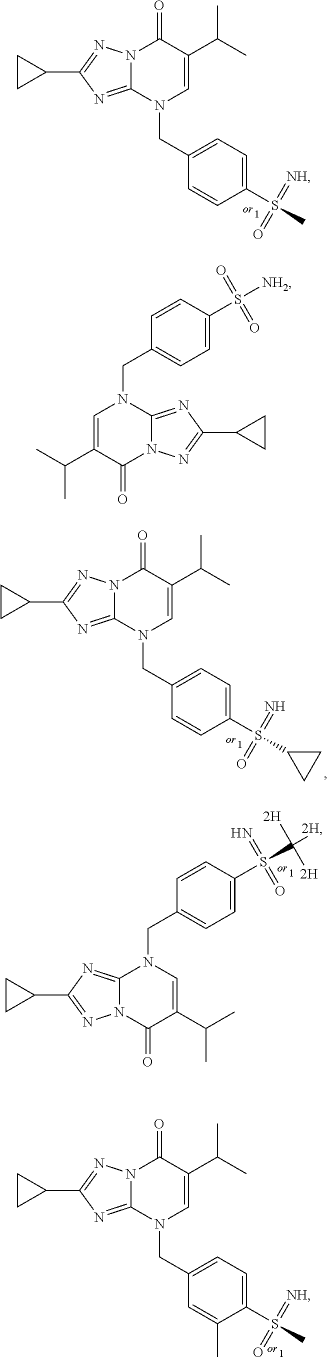

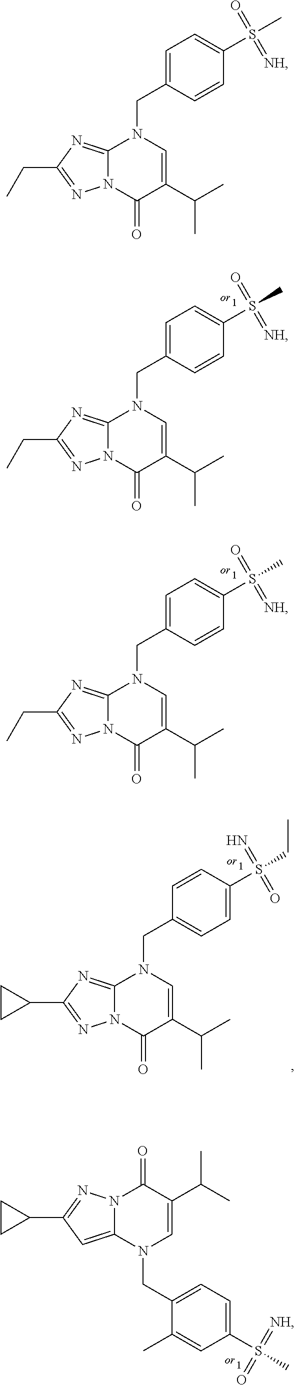

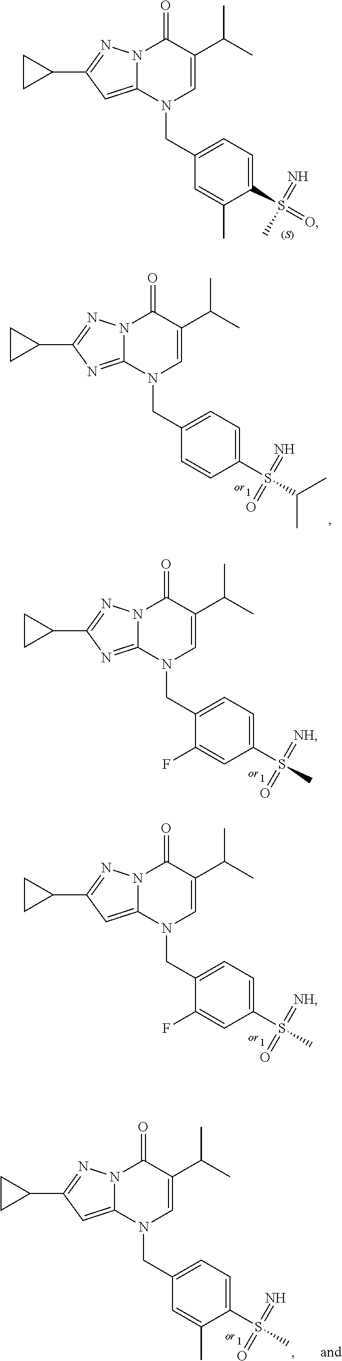

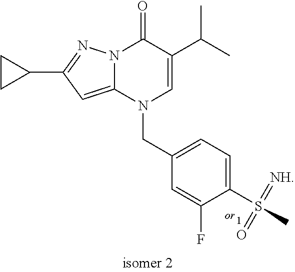 [SMILES: CC(C)c1cn(Cc2ccc([S@](C)(=N)=O)c(F)c2)c2cc(C3CC3)nn2c1=O]

239b. A compound, or a pharmaceutically acceptable salt or solvate thereof, having the following

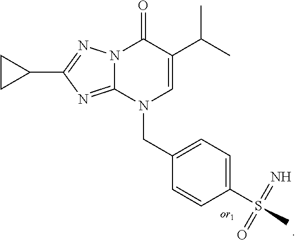 [SMILES: CC(C)c1cn(Cc2ccc([S@](C)(=N)=O)cc2)c2nc(C3CC3)nn2c1=O]

230c. A compound, or a pharmaceutically acceptable salt or solvate thereof, having the following structure:

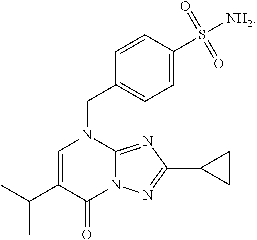 [SMILES: CC(C)c1cn(Cc2ccc(S(N)(=O)=O)cc2)c2nc(C3CC3)nn2c1=O]

230d. A compound, or a pharmaceutically acceptable salt or solvate thereof, having the following structure:

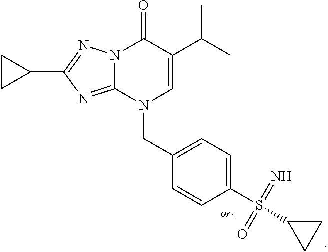 [SMILES: CC(C)c1cn(Cc2ccc([S@@](=N)(=O)C3CC3)cc2)c2nc(C3CC3)nn2c1=O]

230e. A compound, or a pharmaceutically acceptable salt or solvate thereof, having the following

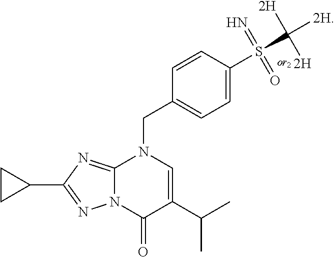

239f. A compound, or a pharmaceutically acceptable salt or solvate thereof, having the following structure:

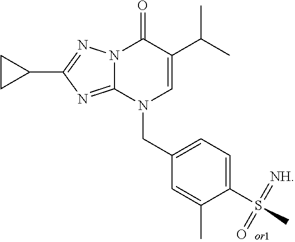 [SMILES: Cc1cc(Cn2cc(C(C)C)c(=O)n3nc(C4CC4)nc23)ccc1[S@](C)(=N)=O]

230g. A compound, or a pharmaceutically acceptable salt or solvate thereof, having the following structure:

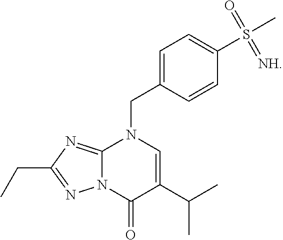 [SMILES: CCc1nc2n(Cc3ccc(S(C)(=N)=O)cc3)cc(C(C)C)c(=O)n2n1]

230h. A compound, or a pharmaceutically acceptable salt or solvate thereof, having the following

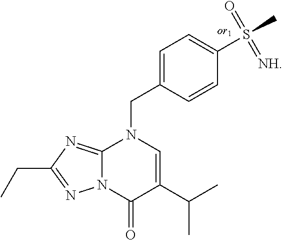 [SMILES: CCc1nc2n(Cc3ccc([S@@](C)(=N)=O)cc3)cc(C(C)C)c(=O)n2n1]

230l. A compound, or a pharmaceutically acceptable salt or solvate thereof, having the following structure:

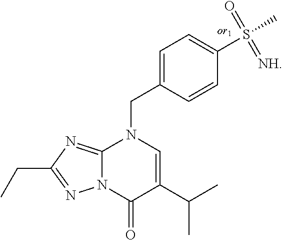 [SMILES: CCc1nc2n(Cc3ccc([S@](C)(=N)=O)cc3)cc(C(C)C)c(=O)n2n1]

239j. A compound, or a pharmaceutically acceptable salt or solvate thereof, having the following structure:

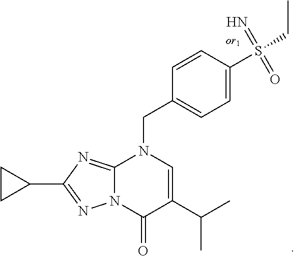 [SMILES: CC[S@](=N)(=O)c1ccc(Cn2cc(C(C)C)c(=O)n3nc(C4CC4)nc23)cc1]

230k. A compound, or a pharmaceutically acceptable salt or solvate thereof, having the following

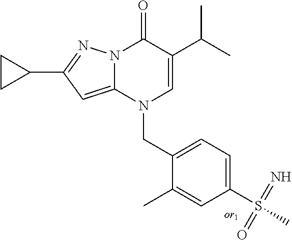 [SMILES: Cc1cc([S@@](C)(=N)=O)ccc1Cn1cc(C(C)C)c(=O)n2nc(C3CC3)cc12]

230l. A compound, or a pharmaceutically acceptable salt or solvate thereof, having the following structure:

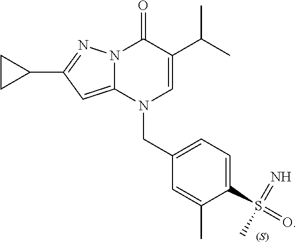

230 m. A compound, or a pharmaceutically acceptable salt or solvate thereof, having the following structure:

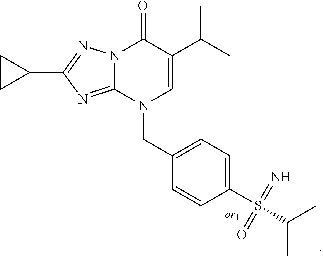 [SMILES: CC(C)c1cn(Cc2ccc([S@@](=N)(=O)C(C)C)cc2)c2nc(C3CC3)nn2c1=O]

230n. A compound, or a pharmaceutically acceptable salt or solvate thereof, having the following

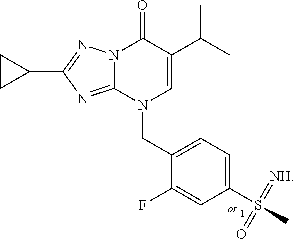 [SMILES: CC(C)c1cn(Cc2ccc([S@](C)(=N)=O)cc2F)c2nc(C3CC3)nn2c1=O]

230o. A compound, or a pharmaceutically acceptable salt or solvate thereof, having the following structure:

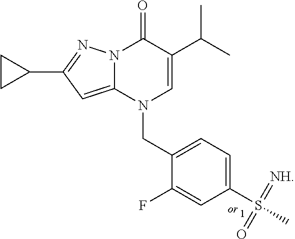 [SMILES: CC(C)c1cn(Cc2ccc([S@@](C)(=N)=O)cc2F)c2cc(C3CC3)nn2c1=O]

238p. A compound, or a pharmaceutically acceptable salt or solvate thereof, having the following structure:

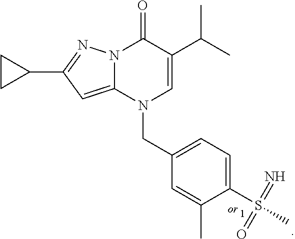 [SMILES: Cc1cc(Cn2cc(C(C)C)c(=O)n3nc(C4CC4)cc23)ccc1[S@@](C)(=N)=O]

230q. A compound, or a pharmaceutically acceptable salt or solvate thereof, having the following

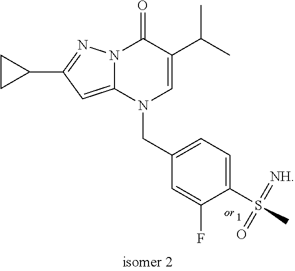 [SMILES: CC(C)c1cn(Cc2ccc([S@](C)(=N)=O)c(F)c2)c2cc(C3CC3)nn2c1=O]

240. A pharmaceutical composition comprising a compound of any one of Clauses 1 to 239q, or a pharmaceutically acceptable salt or solvate thereof, and one or more pharmaceutically acceptable carrier.

241. The compound according to any of Clauses 1 to 239q, or the pharmaceutical composition according to Clause 240 for use in the treatment of a disease or disorder.

242. The compound or pharmaceutical composition for use according to Clause 241, wherein the disease or disorder is a calcium handling or calcification-related disease or disorder.

243. The compound or pharmaceutical composition for use according to Clause 241 or Clause 242, wherein the disease or disorder is selected from the group consisting of: hypophosphatasia (HPP) and cancer.

244. The compound according to any of Clauses 1 to 239q, the compound for use according to any of Clauses 241 to 243, the pharmaceutical composition according to Clause 240, or the pharmaceutical composition for use according to any of Clauses 241 to 243, wherein the compound is an inhibitor of ENPP1.

245. The compound for use according to any of Clauses 241 to 244 or the pharmaceutical composition for use according to any of Clauses 241 to 244, wherein the use is in a method comprising administering the compound according to any of Formulas (I) to (VIII) in combination with one or more additional therapeutic agent.

246. A method for treating a disease or disorder in a subject in need thereof, the method comprising administering to the subject, a therapeutically effective amount of a compound according to any of Clauses 1 to 239q, or a pharmaceutical composition according to Clause 240.

247. The method according to Clause 246, wherein the disease or disorder is a calcium handling or calcification-related disease or disorder.

248. The method according to Clause 246 or Clause 247, wherein the disease or disorder is selected from the group consisting of: hypophosphatasia (HPP) and cancer.

249. The method according to any of Clauses 246 to 248, wherein the compound is an inhibitor of ENPP1.

250. The method according to any of Clauses 246 to 249, wherein the method comprises administering the compound according to any of formulas (I) to (VIII) in combination with one or more additional therapeutic agent.

The skilled person will appreciate that many modifications may be made to the above examples, the aspects and the embodiments disclosed herein without departing from the scope of the present invention as defined in the accompanying claims and/or the above clauses.

1. A compound of formula (I) or a pharmaceutically acceptable salt or solvate thereof: 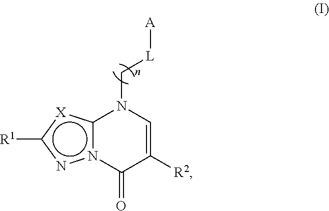
wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
R2 is selected from C1-C6 alkyl, C1-C6 haloalkyl, C3-C6 cycloalkyl, phenyl, and 6-membered heteroaryl having one or more (e.g. 1, 2, 3, or 4) ring heteroatoms independently selected from N, O, and S, wherein each cycloalkyl, phenyl or heteroaryl is optionally substituted with one or more (e.g., 1, 2, 3, or 4) halo;
n is 1, 2, or 3;
m is 0 or 1;
L is selected from phenyl, 6-membered heteroaryl having one or more (e.g. 1, 2, 3, or 4) ring heteroatoms independently selected from N, O, and S, C3-C7 cycloalkyl, 3-7-membered heterocycloalkyl having one or more (e.g. 1, 2, 3, or 4) ring heteroatoms independently selected from N, O, and S, C9-C10 bicyclic aryl, and 9-10-membered bicyclic heteroaryl having one or more (e.g. 1, 2, 3, or 4) ring heteroatoms independently selected from N, O, and S, wherein each phenyl, heteroaryl, cycloalkyl, heterocycloalkyl, bicyclic aryl or bicyclic heteroaryl is optionally substituted with one or more (e.g., 1, 2, 3, 4, or 5) substituents independently selected from R6 and R7;
each R6 and R7 are independently selected from halo, hydroxy, C1-C4 alkoxyl, C1-C4 haloalkoxyl, C1-C6 alkyl, C1-C6 haloalkyl, —NR11₂, C1-C3 alkoxyl-C1-C3 alkyl, phenyl, and 6-membered heteroaryl having one or more (e.g. 1, 2, 3, or 4) ring heteroatoms independently selected from N, O, and S, wherein each phenyl or heteroaryl is optionally substituted with one or more (e.g., 1, 2, 3, or 4) substituents independently selected from halo and Me;
each R11 is independently selected from H and C1-C3 alkyl;
A is selected from —SO₂NH₂ and —S(O)(NH)R13;
R13 is C1-C3 alkyl, C1-C3 haloalkyl, or C3-C6 cycloalkyl, wherein cycloalkyl is optionally substituted with one or more (e.g., 1, 2, 3, or 4) halo; or
when L is phenyl or heteroaryl, R13 and R6 or R7 together form a 5-6 membered heterocycloalkyl optionally having one or more (e.g., 1, 2, or 3) additional ring heteroatoms independently selected from N, S and O.

2. The compound of claim 1, wherein X is N.

3. The compound of claim 1, wherein X is CR5, and R5 is H or halo.

4. The compound of claim 1, wherein R1 is H.

5. The compound of claim 1, wherein R1 is selected from:

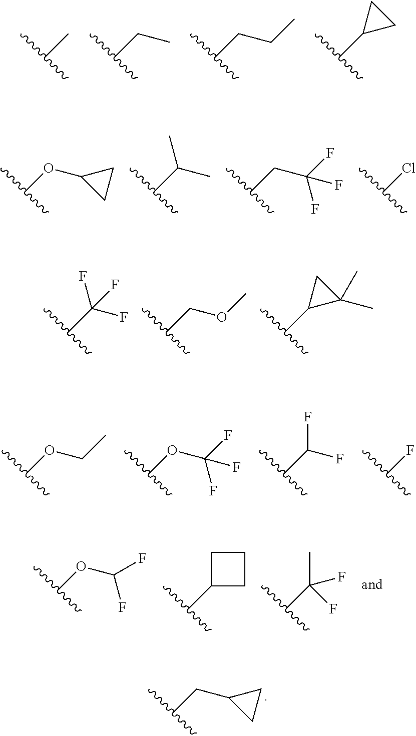

6. The compound of claim 1, wherein R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl.

7. The compound of claim 1, wherein:
R2 is selected from: 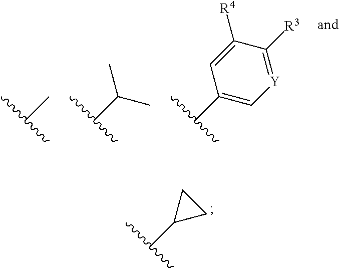
Y is CH or N;
R3 is H or halo; and
R4 is H or halo, wherein halo is optionally F.

8. (canceled)

9. The compound of claim 1, wherein
L-A is selected from: 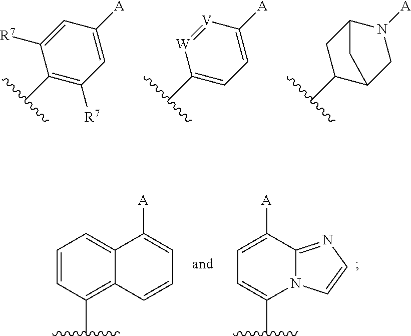
V is CR6 or N;
W is CR7 or N; and
each R6 and R7 is independently selected from H, halo, hydroxy, C1-C4 alkoxy, C1-C4 haloalkoxyl, C1-C6 alkyl, C1-C6 haloalkyl, C1-C3 alkoxyl-C1-C3 alkyl, phenyl, 6-membered heteroaryl having one or more (e.g. 1, 2, 3, or 4) ring heteroatoms independently selected from N, O, and S, —NH₂, —NHMe, and —NMe₂, wherein each phenyl or heteroaryl is optionally substituted with one or more (e.g., 1, 2, 3, or 4) halo.

10. The compound of claim 1, wherein
L-A is: 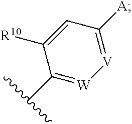
V is N or CR6;
W is N or CR7;
R6 is H, halo, hydroxy, C1-C4 alkyl, C1-C4 haloalkyl, C1-C4 alkoxyl, C1-C4 haloalkoxyl, C1-C3 alkoxyl-C1-C3 alkyl, phenyl, or 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R7 is H, C1-C4 alkyl, hydroxy, —N(H)(R11), or halo;
R10 is H or halo; and
R11 is H or C1-C3 alkyl.

11. (canceled)

12. (canceled)

13. The compound of claim 9, wherein R6 is H or is selected from:

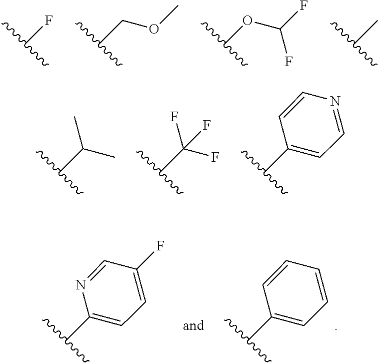

14. The compound of claim 1, wherein L-A is selected from:

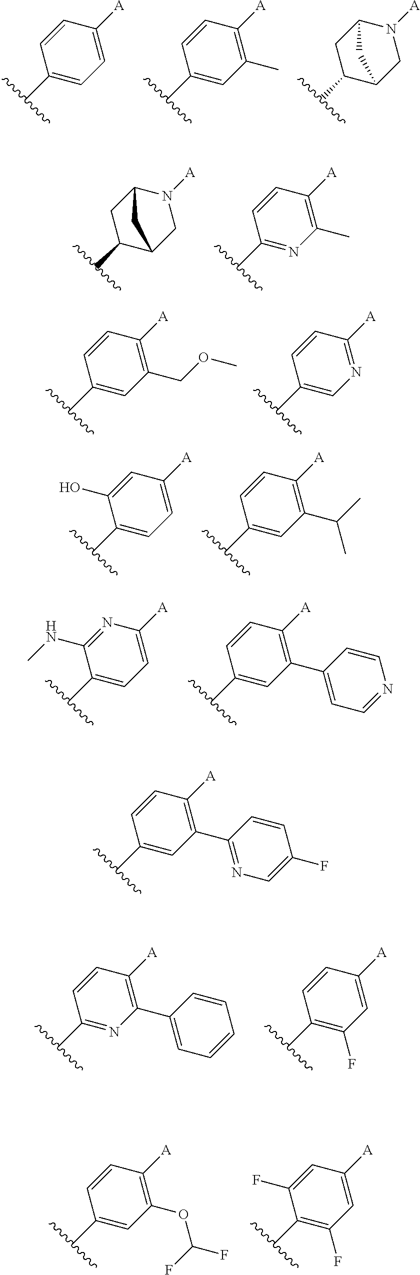

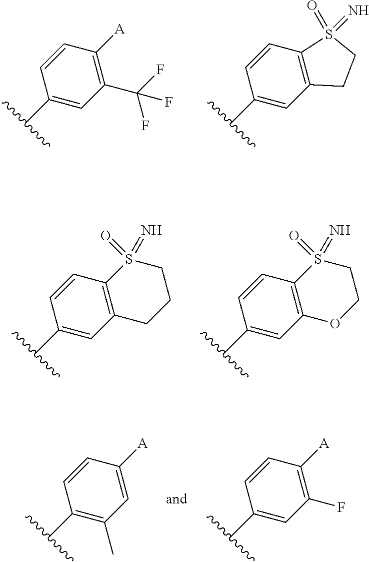

15. (canceled)

16. (canceled)

17. (canceled)

18. The compound of claim 1, wherein A is —SO₂NH₂.

19. (canceled)

20. The compound of claim 1, wherein A is-S(O)(NH)R13, wherein R13 is C1-C3 alkyl (e.g. C₁H³ or C²H₃), C1-C3 haloalkyl, or C3-C6 cycloalkyl, and cycloalkyl is optionally substituted with one or more (e.g., 1, 2, 3, or 4) halo.

21. (canceled)

22. The compound of claim 20, wherein A is selected from:

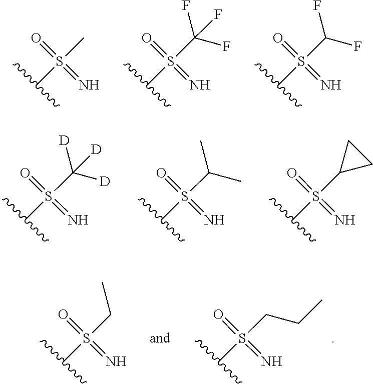

23. (canceled)

24. The compound of claim 1, having a structure of formula (II) or a pharmaceutically acceptable salt or solvate thereof: 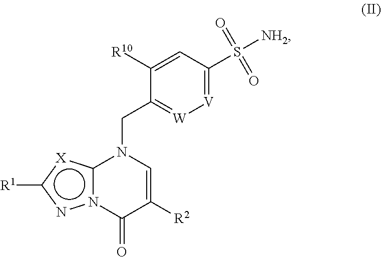
wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
n is 1, 2, or 3;
m is 0 or 1;
V is N or CR6;
W is N or CR7;
each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R10 is H or halo; and
R11 is H or C1-C3 alkyl.

25. The compound of claim 1, having a structure of formula (III) or a pharmaceutically acceptable salt or solvate thereof: 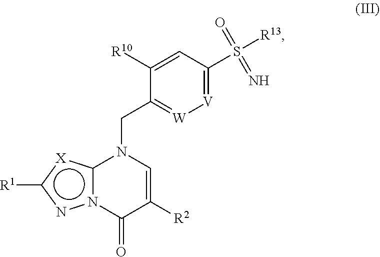
wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
n is 1, 2, or 3;
m is 0 or 1;
V is N or CR6;
W is N or CR7;
each R6 and R7 is independently selected from H, halo, hydroxy, C1-C4 alkyl, C1-C4 haloalkyl, C1-C4 alkoxy, C1-C4 haloalkoxy, —N(H)(R11), C1-C3 alkoxyl-C1-C3 alkyl, phenyl, and 6-membered heteroaryl having 1, 2 or 3 ring heteroatoms independently selected from N, S and O, wherein each phenyl or heteroaryl is optionally substituted with one or more (e.g., 1, 2, 3, or 4) halo;
R10 is H or halo;
R11 is H or C1-C3 alkyl; and
R13 is C1-C3 alkyl, C1-C3 haloalkyl, or C3-C6 cycloalkyl, wherein cycloalkyl is optionally substituted with one or more (e.g., 1, 2, 3, or 4) halo; or
R13 and R6 together form a 5-6 membered heterocycloalkyl optionally having one or more (e.g., 1, 2 or 3) additional ring heteroatoms independently selected from N, S and O.

26. The compound of claim 1, having a structure of formula (IV) or (V) or a pharmaceutically acceptable salt or solvate thereof: 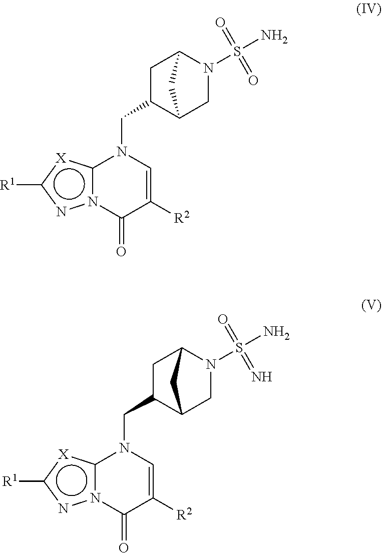
wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo; and
m is 0 or 1.

27. The compound of claim 1, having a structure of formula (VI) or (VII) or a pharmaceutically acceptable salt or solvate thereof: 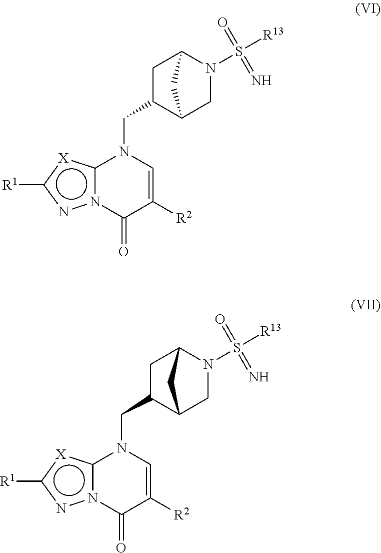
wherein:
X is N or C(CH₂)ₘR5;
R5 is selected from H, halo, Me, CN, and OMe;
R1 is selected from H, halo, C1-C6 alkyl, C1-C6 haloalkyl, C3-C6 cycloalkyl, C3-C6 cycloalkyl-O—, C1-C6 alkoxyl, C1-C6 haloalkoxyl, and C1-C3 alkoxyl-C1-C3 alkyl, wherein cycloalkyl is optionally substituted with one or more (e.g., 1, 2, 3, or 4) substituents independently selected from C1-C3 alkyl, C1-C3 alkoxyl, C3-C6 cycloalkyl and halo; or
R5 and R1 taken together with the atoms to which they are attached form a 5-6 membered non-aromatic ring having zero or more (e.g. 0, 1, or 2) ring heteroatoms independently selected from N, O, and S, a 6 membered aromatic ring having zero or more (e.g. 0, 1, or 2) ring heteroatoms independently selected from N, O, and S, or a 5 membered aromatic ring having one or more (e.g. 1 or 2) ring heteroatoms independently selected from N, O, and S, wherein the aromatic or non-aromatic ring is optionally substituted with one or more (e.g., 1, 2, 3, or 4) substituents independently selected from halo and C1-C6 alkoxyl;
R2 is selected from C1-C6 alkyl, C1-C6 haloalkyl, C3-C6 cycloalkyl, phenyl, and 6-membered heteroaryl having 1, 2, 3, or 4 ring heteroatoms independently selected from N, O, and S, wherein each cycloalkyl, phenyl or heteroaryl is optionally substituted with one or more (e.g., 1, 2, 3, or 4) halo;
m is 0 or 1; and
R13 is C1-C3 alkyl, C1-C3 haloalkyl, or C3-C6 cycloalkyl, wherein cycloalkyl is optionally substituted with one or more (e.g., 1, 2, 3, or 4) halo.

28. (canceled)

29. (canceled)

30. (canceled)

31. (canceled)

32. A pharmaceutical composition comprising a compound of claim 1, or a pharmaceutically acceptable salt or solvate thereof, and one or more pharmaceutically acceptable carrier.

33. (canceled)

34. A method of treating a subject suffering from a disease or disorder, comprising administering to the subject a compound of claim 1, or a pharmaceutically acceptable salt or solvate thereof in an amount effective to treat the disease or disorder, wherein the disease or disorder is a calcium handling or calcification-related disease or disorder.

35. The method of claim 34, wherein the disease or disorder is selected from the group consisting of: hypophosphatasia (HPP) and cancer.

36. (canceled)

37. The method of claim 34, further comprising administering one or more additional therapeutic agent to the subject.
